(19) United States
(12) Patent Application Publication
(10) Pub. No.: US 2022/0133790 A1
(43) Pub. Date: May 5, 2022

(54) MODIFIED IMMUNE CELLS HAVING ENHANCED ANTI-NEOPLASIA ACTIVITY AND IMMUNOSUPPRESSION RESISTANCE

(71) Applicant: BEAM THERAPEUTICS INC., Cambridge, MA (US)

(72) Inventors: Jason Michael GEHRKE, Cambridge, MA (US); Aaron D. EDWARDS, Cambridge, MA (US); Ryan MURRAY, Cambridge, MA (US)

(73) Assignee: BEAM THERAPEUTICS INC., Cambridge, MA (US)

(21) Appl. No.: 17/423,428

(22) PCT Filed: Jan. 16, 2020
(86) PCT No.: PCT/US20/13964

§ 371 Date: Jul. 15, 2021

Related U.S. Application Data
(60) Provisional application No. 62/793,277, filed on Jan. 16, 2019.
(60) Provisional application No. 62/839,870, filed on Apr. 29, 2019.

(51) Int. Cl. A61K 35/17; C12N 5/0783; C12N 9/22; C12N 15/10; C12N 15/113

(52) U.S. Cl. CPC A61K 35/17; C12N 5/0636; C12N 9/22; C12N 15/102; C12N 2310/20; C12Y 305/04004; C12Y 305/04005; C12N 2510/00; C12N 15/113

(57) ABSTRACT

As described below, the present invention features genetically modified immune cells having enhanced anti-neoplasia activity, resistance to immune suppression, and decreased risk of eliciting a graft versus host reaction, or a combination thereof. The present invention also features methods for producing and using these modified immune effector cells.

### INCORPORATION BY REFERENCE

This application claims the benefit of U.S. Provisional Application No. 62/793,277 filed on Jan. 16, 2019 and U.S. Provisional Application No. 62/839,870 filed on Apr. 29, 2019.

### BACKGROUND OF THE INVENTION

Autologous and allogeneic immunotherapies are neoplasia treatment approaches in which immune cells expressing chimeric antigen receptors are administered to a subject. To generate an immune cell that expresses a chimeric antigen receptor (CAR), the immune cell is first collected from the subject (autologous) or a donor separate from the subject receiving treatment (allogeneic) and genetically modified to express the chimeric antigen receptor. The resulting cell expresses the chimeric antigen receptor on its cell surface (e.g., CAR T-cell), and upon administration to the subject, the chimeric antigen receptor binds to the marker expressed by the neoplastic cell. This interaction with the neoplasia marker activates the CAR-T cell, which then cell kills the neoplastic cell. But for autologous or allogeneic cell therapy to be effective and efficient, significant conditions and cellular responses, such as T cell signaling inhibition, must be overcome or avoided. For allogeneic cell therapy, graft versus host disease and host rejection of CAR-T cells may provide additional challenges. Editing genes involved in these processes can enhance CAR-T cell function and resistance to immunosuppression or inhibition, but current methodologies for making such edits have the potential to induce large, genomic rearrangements in the CAR-T cell, thereby negatively impacting its efficacy. Thus, there is a significant need for techniques to more precisely modify immune cells, especially CAR-T cells. This application is directed to this and other important needs.

### SUMMARY OF THE INVENTION

As described below, the present invention features genetically modified immune cells having enhanced anti-neoplasia activity, resistance to immune suppression, and decreased risk of eliciting a graft versus host reaction, or host versus graft reaction where host CD8⁺ T cells recognize a graft as non-self (e.g., where a transplant recipient generates an immune response against the transplanted organ), or a combination thereof. In one embodiment, a subject having or having a propensity to develop graft versus host disease (GVHD) is administered a CAR-T cell that lacks or has reduced levels of functional TRAC. In one embodiment, a subject having or having a propensity to develop host versus graft disease (HVGD) is administered a CAR-T cell that lacks or has reduced levels of functional beta2 microglobulin (B2M). The present invention also features methods for producing and using these modified immune cells.

In one aspect, provided herein is a method for producing a modified immune cell with reduced immunogenicity and/or increased anti-neoplasia activity by multiplexed editing, the method comprising: modifying at least four gene sequences or regulatory elements thereof, at a single target nucleobase in each thereof in an immune cell, thereby generating the modified immune cell with reduced immunogenicity and/or increased anti-neoplasia activity.

In another aspect, provided herein is a method for producing a population of modified immune cells with reduced immunogenicity and/or increased anti-neoplasia activity by multiplexed editing, the method comprising: modifying at least four gene sequences or regulatory elements thereof at a single target nucleobase in each thereof in a population of immune cells, thereby generating the population of modified immune cells with reduced immunogenicity and/or increased anti-neoplasia activity.

In some embodiments, the at least one of the at least four gene sequences is a checkpoint inhibitor gene sequence, an immune response regulation gene sequence, or an immunogenic gene sequence.

In some embodiments, the modifying reduces expression of at least one of the at least four gene sequences.

In some embodiments, the expression of at least one of the at least four genes is reduced by at least 80% as compared to a control cell without the modification.

In some embodiments, the expression of each one of the at least four genes is reduced by at least 80% as compared to a control cell without the modification.

In some embodiments, the expression of at least one of the at least four genes is reduced in at least 50% of the population of immune cells.

In some embodiments, the expression of each one of the at least four genes is reduced in at least 50% of the population of immune cells.

In some embodiments, the at least four gene sequences comprise a TRAC gene sequence.

In some embodiments, the at least four gene sequences comprise a check point inhibitor gene sequence.

In some embodiments, the at least four gene sequences comprise a PDCD1 gene sequence.

In some embodiments, the at least four gene sequences comprise a T cell marker gene sequence.

In some embodiments, the at least four gene sequences comprise a CD52 gene sequence.

In some embodiments, the at least four gene sequences comprises a CD7 gene sequence.

In some embodiments, the at least four gene sequences comprise a TRAC gene sequence, a PDCD1 gene sequence, a CD52 gene sequence, or a CD7 gene sequence.

In some embodiments, the at least four sequences comprise a TCR complex gene sequence, a CD7 gene sequence, a CD52 gene sequence, and a gene sequence selected from the group consisting of CIITA a CD2 gene sequence, a CD4 gene sequence, a CD5 gene sequence, a CD7 gene sequence, a CD30 gene sequence, a CD33 gene sequence, a CD52 gene sequence, a CD70 gene sequence, a B2M gene sequence, and a CIITA gene sequence

In some embodiments, the at least four gene sequences comprise a gene sequence selected from the group consisting of a CD2 gene sequence, a TRAC gene sequence, a CD3 epsilon gene sequence, a CD3 gamma gene sequence, a CD3 delta gene sequence, a TRBC1 gene sequence, a TRBC2 gene sequence, a CD4 gene sequence, a CD5 gene sequence, a CD7 gene sequence, a CD30 gene sequence, a CD33 gene sequence, a CD52 gene sequence, a CD70 gene sequence, a B2M gene sequence, and a CIITA gene sequence.

The method of some embodiments described herein comprises modifying five gene sequences or regulatory elements thereof at a single target nucleobase in each thereof in the immune cell.

The method of some embodiments described herein comprises modifying six gene sequences or regulatory elements thereof at a single target nucleobase in each thereof in the immune cell.

The method of some embodiments described herein comprises modifying seven gene sequences or regulatory elements thereof at a single target nucleobase in each thereof in the immune cell.

The method of some embodiments described herein comprises modifying eight gene sequences or regulatory elements thereof at a single target nucleobase in each thereof in the immune cell.

The method of some embodiments described herein comprises modifying five gene sequences or regulatory elements thereof at a single target nucleobase in each thereof in the population of immune cells.

The method of some embodiments described herein comprises modifying six gene sequences or regulatory elements thereof at a single target nucleobase in each thereof in the population of immune cells.

The method of some embodiments described herein comprises modifying seven gene sequences or regulatory elements thereof at a single target nucleobase in each thereof in the population of immune cells.

The method of some embodiments described herein modifying eight gene sequences or regulatory elements thereof at a single target nucleobase in each thereof in the population of immune cells.

In some embodiments, the five, six, seven, or eight gene sequences or regulatory elements thereof are selected from the group consisting of a CD2 gene sequence, a TRAC gene sequence, a CD3 epsilon gene sequence, a CD3 gamma gene sequence, a CD3 delta gene sequence, a TRBC1 gene sequence, a TRBC2 gene sequence, a CD4 gene sequence, a CD5 gene sequence, a CD7 gene sequence, a CD30 gene sequence, a CD33 gene sequence, a CD52 gene sequence, a CD70 gene sequence, a B2M gene sequence, and a CIITA gene sequence.

In some embodiments, the five, six, seven, or eight gene sequences or regulatory elements thereof at comprises a CD3 gene sequence, a CD7 gene sequence, a CD2 gene sequence, a CD5 gene sequence, and a CD52 gene sequence.

In some embodiments, the modifying comprises deaminating the single target nucleobase.

In some embodiments, the deaminating is performed by a polypeptide comprising a deaminase.

In some embodiments, the deaminase is associated with a nucleic acid programmable DNA binding protein (napDNAbp) to form a base editor.

In some embodiments, the deaminase is fused to the nucleic acid programmable DNA binding protein (napDNAbp).

In some embodiments, the napDNAbp comprises a Cas9 polypeptide or a portion thereof.

In some embodiments, the napDNAbp comprises a Cas9 nickase or nuclease dead Cas9.

In some embodiments, the deaminase is a cytidine deaminase.

In some embodiments, the single target nucleobase is a cytosine (C) and wherein the modification comprises conversion of the C to a thymine (T).

In some embodiments, the base editor further comprises a uracil glycosylase inhibitor.

In some embodiments, the deaminase is an adenosine deaminase.

In some embodiments, the single target nucleobase is a adenosine (A) and wherein the modification comprises conversion of the A to a guanine (G).

In some embodiments, the modifying comprises contacting the immune cell with a guide nucleic acid sequences.

In some embodiments, the modifying comprises contacting the immune cell with at least four guide nucleic acid sequences, wherein each guide nucleic acid sequence targets the napDNAbp to one of the at least four gene sequences or regulatory elements thereof.

In some embodiments, the guide nucleic acid sequence comprises a sequence selected from guide RNA sequences of table 8A, table 8B, or table 8C.

In some embodiments, the guide nucleic acid sequence comprises a sequence selected from the group consisting of UUCGUAUCUGUAAAACCAAG, CCUACCUGUCACCAGGACCA, CUCUUACCUGUACCAUAACC, CACCUACCUAAGAACCAUCC, ACUCACGCUGGAUAGCCUCC, ACUCACCCAGCAUCCCCAGC, CACUCACCUUAGCCUGAGCA, and CACGCACCUGGACAGCUGAC.

In some embodiments, the modifying comprises replacing the single target nucleobase with a different nucleobase by target-primed reverse transcription with a reverse transcriptase and an extended guide nucleic acid sequence.

In some embodiments, the extended guide nucleic acid sequence comprises a reverse transcription template sequence, a reverse transcription primer binding site, or a combination thereof.

In some embodiments, the single target nucleobase is in an exon.

In some embodiments, modifying generates a premature stop codon in the exon.

In some embodiments, the single target nucleobase is within an exon 1, an exon 2, or an exon 3 of the TRAC gene sequence.

In some embodiments, the single target nucleobase is within an exon 1, an exon 2, or an exon 5 of the PCDC1 gene sequence.

In some embodiments, the single target nucleobase is within an exon 1 or an exon 2 of the CD52 gene sequence.

In some embodiments, the single target nucleobase is within an exon 1, an exon 2, or an exon 3 of the CD7 gene sequence.

In some embodiments, the single target nucleobase is within an exon 1 or an exon 2 of the B2M gene sequence.

In some embodiments, the single target nucleobase is within an exon 2, an exon 3, an exon 4, an exon 5, an exon 6, an exon 7, or an exon 8 of the CD5 gene sequence.

In some embodiments, the single target nucleobase is within an exon 2, an exon 3, an exon 4, or an exon 5 of the CD2 gene sequence.

In some embodiments, the single target nucleobase is within an exon 1, an exon 2, an exon 4, an exon 7, an exon 8, an exon 9, an exon 10, an exon 11, an exon 12, an exon 14, an exon 15, an exon 18, or an exon 19 of the CIITA gene sequence.

In some embodiments, the single target nucleobase is in a splice donor site or a splice acceptor site.

In some embodiments, the single target nucleobase is in an exon 1 splice acceptor site, an exon 1 splice donor site, or an exon 3 splice acceptor site of the TRAC gene sequence.

In some embodiments, the single target nucleobase is in an exon 1 splice acceptor site, an exon 1 splice donor site, an exon 2 splice acceptor site, an exon 3 splice donor site, an exon 4 splice acceptor site, an exon 4 splice donor site, or an exon 5 splice acceptor site of the PDCD1 gene sequence.

In some embodiments, the single target nucleobase is in an exon 1 splice donor site, or an exon 2 splice acceptor site of the CD52 gene sequence.

In some embodiments, the single target nucleobase is in an exon 1 splice donor site, an exon 2 splice donor site, an exon 2 splice acceptor site, or an exon 3 splice acceptor site of the CD7 gene sequence.

In some embodiments, the single target nucleobase is in an exon 1 splice donor site, an exon 2 splice donor site, an exon 2 splice acceptor site, or an exon 3 splice acceptor site of the B2M gene sequence.

In some embodiments, the single target nucleobase is in an exon 3 splice donor site of the CD2 gene sequence.

In some embodiments, the single target nucleobase is in an exon 1 splice donor site, an exon 1 splice acceptor site, an exon 3 splice acceptor site, an exon 3 splice donor site, an exon 4 splice acceptor site, an exon 5 splice donor site, an exon 6 splice acceptor site, an exon 9 splice donor site, an exon 10 splice acceptor site of the CD5 gene sequence.

In some embodiments, the single target nucleobase is in an exon 1 splice donor site, an exon 7 splice donor site, an exon 8 splice acceptor site, an exon 9 slice donor site, an exon 10 splice acceptor site, an exon 11 splice acceptor site, an exon 14 splice acceptor site, an exon 14 splice donor site, an exon 15 splice donor site, an exon 16 splice acceptor site, an exon 16 splice donor site, an exon 17 splice acceptor site, an exon 17 splice donor site, or an exon 19 splice acceptor site of the CIITACIITA gene sequence.

In some embodiments, the immune cell is a human cell. In some embodiments, the immune cell is a cytotoxic T cell, a regulatory T cell, a T helper cell, a dendritic cell, a B cell, or a NK cell.

In some embodiments, the population of immune cells are human cells.

In some embodiments, the population of immune cells are cytotoxic T cells, regulatory T cells, T helper cells, dendritic cells, B cells, or NK cells.

In some embodiments, the modifying is ex vivo.

In some embodiments, the immune cell or the population of immune cells are derived from a single human donor.

In some embodiments, the method further comprising contacting the immune cell or the population of immune cells with a polynucleotide that encodes an exogenous functional chimeric antigen receptor (CAR) or a functional fragment thereof.

In some embodiments, contacting the immune cell or the population of immune cells with a lentivirus comprising the polynucleotide that encodes the CAR.

In some embodiments, contacting the immune cell or the population of immune cells with a napDNAbp and a donor DNA sequence comprising the polynucleotide that encodes the CAR.

In some embodiments, the napDNAbp is a Cas12b.

In some embodiments, the CAR specifically binds a marker associated with neoplasia.

In some embodiments, the neoplasia is a T cell cancer, a B cell cancer, a lymphoma, a leukemia, or a multiple myeloma.

In some embodiments the CAR specifically binds CD7.

In some embodiments, the CAR specifically binds BCMA.

In some embodiments, the immune cell or the population of immune cells comprises no detectable translocation. In some embodiments, at least 50% of the population of immune cells express the CAR. In some embodiments, at least 50% of the population of immune cells are viable. In some embodiments, at least 50% of the population of immune cells expand at least 80% of expansion rate of a population of control cells of a same type without the modification.

In the method of some embodiments described herein, the modifying generates less than 10% of indels in the immune cell. In some embodiments, the modifying generates less than 5% of non-target edits in the immune cell. In some embodiments, the modifying generates less than 5% of off-target edits in the immune cell.

In one aspect, provided herein is a modified immune cell produced according to some embodiments described in the preceding paragraphs.

In one aspect, provided herein is a population of modified immune cells produced according to some embodiments described in the preceding paragraphs.

In another aspect, provided herein is a modified immune cell with reduced immunogenicity or increased anti-neoplasia activity, wherein the modified immune cell comprises a single target nucleobase modification in each one of at least four gene sequences or regulatory elements thereof. In some embodiments, in the modified immune cell described above, each one of the at least four gene sequences is a checkpoint inhibitor gene sequence, an immune response regulation gene sequence, or an immunogenic gene sequence.

In the modified immune cell of the preceding embodiments the at least four gene sequences comprise a TCR complex gene sequence.

In some embodiments, the at least four gene sequences comprise a TRAC gene sequence. In some embodiments, the at least four gene sequences comprise a check point inhibitor gene sequence. In some embodiments, the at least four gene sequences comprise a PDCD1 gene sequence.

In some embodiments, the at least four gene sequences comprise a T cell marker gene sequence.

In some embodiments, the at least four gene sequences comprise CD52 gene sequence.

In some embodiments, the at least four gene sequences comprises a CD7 gene sequence.

In some embodiments, the expression of one of the at least four genes is reduced by at least 80% as compared to a control cell without the modification.

In some embodiments, the expression of each one of the at least four genes is reduced by at least 90% as compared to a control cell without the modification.

In some embodiments, the immune cell comprises a modification at a single target nucleobase in each one of five gene sequences or regulatory elements thereof, wherein each one of the five gene sequences is a checkpoint inhibitor gene sequence, an immune response regulation gene sequence, or an immunogenic gene sequence.

In some embodiments, the immune cell comprises a modification at a single target nucleobase in each one of six gene sequences or regulatory elements thereof, wherein each one of the six gene sequences is a checkpoint inhibitor gene sequence, an immune response regulation gene sequence, or an immunogenic gene sequence.

In some embodiments, the immune cell comprises a modification at a single target nucleobase in each one of seven gene sequences or regulatory elements thereof, wherein each one of the seven gene sequences is a checkpoint inhibitor gene sequence, an immune response regulation gene sequence or an immunogenic gene sequence.

In some embodiments, the immune cell comprises a modification at a single target nucleobase in each one of eight gene sequences or regulatory elements thereof, wherein each one of the eight gene sequences is a checkpoint inhibitor gene sequence, an immune response regulation gene sequence, or an immunogenic gene sequence.

In some embodiments, the expression of at least one of the five, six, seven or eight genes is reduced by at least 90% as compared to a control cell without the modification.

In some embodiments, the expression of each one of the five, six, seven, or eight genes is reduced by at least 90% as compared to a control cell without the modification.

In some embodiments, the five, six, seven, or eight gene sequences or regulatory elements thereof comprise a sequence selected from the group consisting of a CD2 gene sequence, a TRAC gene sequence, a CD3 epsilon gene sequence, a CD3 gamma gene sequence, a CD3 delta gene sequence, a TRBC1 gene sequence, a TRBC2 gene sequence, a CD4 gene sequence, a CD5 gene sequence, a CD7 gene sequence, a CD30 gene sequence, a CD33 gene sequence, a CD52 gene sequence, a CD70 gene sequence, a B2M gene sequence, and a CIITA gene sequence.

In one aspect, provided herein is a modified immune cell comprising a single target nucleobase modification in each one of a CD3 gene sequence, a CD5 gene sequence, a CD52 gene sequence, and a CD7 gene sequence, wherein the modified immune cell exhibits reduced immunogenicity or increased anti-neoplasia activity as compared to a control cell of a same type without the modification.

In some embodiments, the modified immune cell further comprises a single target nucleobase modification in a CD2 gene sequence, CIITA or a regulatory element of each thereof.

In some embodiments, the modified immune cell comprises a single target nucleobase modification in a TRAC gene sequence, a CD3 epsilon gene sequence, a CD3 gamma gene sequence, a CD3 delta gene sequence, a TRBC1 gene sequence, or a TRBC2 gene sequence further comprises a single target nucleobase modification in a gene sequence a CD4 gene sequence, a CD30 gene sequence, a CD33 gene sequence, a CD70 gene sequence, a B2M gene sequence, and a CIITA gene sequence or a regulatory element of each thereof.

In some embodiments, the modified immune cell comprises a single nucleobase modification in each one of a TRAC gene sequence, a PDCD1 gene sequence, a CD52 gene sequence, a CD7 gene sequence, a CD2 gene sequence, a CD5 gene sequence, a CIITA gene sequence, and a B2M gene sequence.

In some embodiments, the modified immune cell comprises no detectable translocation.

In some embodiments, the modified immune cell comprises less than 1% of indels.

In some embodiments, the modified immune cell comprises less than 5% of non-target edits.

In some embodiments, the modified immune cell comprises less than 5% of off-target edits.

In some embodiments, the modified immune has increased growth or viability compared to a reference cell. In some embodiments, the reference cell is an immune cell modified with a Cas9 nuclease.

In some embodiments, the modified immune cell is a mammalian cell.

In some embodiments, the modified immune cell is a human cell.

In some embodiments, the modified immune cell is a cytotoxic T cell, a regulatory T cell, a T helper cell, a dendritic cell, a B cell, or a NK cell.

In some embodiments, the modified the immune cell is in an ex vivo culture.

In some embodiments, the modified the immune cell is derived from a single human donor.

In some embodiments, the modified the immune cell further comprises a polynucleotide that encodes an exogenous functional chimeric antigen receptor (CAR) or a functional fragment thereof.

In some embodiments, the polynucleotide that encodes the CAR is integrated in the genome of the immune cell.

In some embodiments, the CAR specifically binds a marker associated with neoplasia.

In some embodiments, the neoplasia is a T cell cancer, a B cell cancer, a lymphoma, a leukemia, or a multiple myeloma.

In some embodiments, the CAR specifically binds CD7.

In some embodiments, the CAR specifically binds BCMA.

In some embodiments, the single target nucleobase is in an exon.

In some embodiments, the single target nucleobase is within an exon 1, an exon 2, or an exon 3 of the TRAC gene sequence.

In some embodiments, the single target nucleobase is within an exon 1, an exon 2, or an exon 5 of the PCDC1 gene sequence.

In some embodiments, the single target nucleobase is within an exon 1 or an exon 2 of the CD52 gene sequence.

In some embodiments, the single target nucleobase is within an exon 1, an exon 2, or an exon 3 of a CD7 gene sequence.

In some embodiments, the single target nucleobase is in a splice donor site or a splice acceptor site.

In some embodiments, the single target nucleobase is in an exon 1 splice acceptor site, an exon 1 splice donor site, or an exon 3 splice acceptor site of the TRAC gene sequence.

In some embodiments, the single target nucleobase is in an exon 1 splice acceptor site, an exon 1 splice donor site, an exon 2 splice acceptor site, an exon 3 splice donor site, an exon 4 splice acceptor site, an exon 4 splice donor site, or an exon 5 splice acceptor site of the PDCD1 gene sequence.

In some embodiments, the single target nucleobase is in an exon 1 splice donor site, or an exon 2 splice acceptor site of the CD52 gene sequence.

In some embodiments, the single target nucleobase is in an exon 1 splice donor site, an exon 2 splice donor site, an exon 2 splice acceptor site, or an exon 3 splice acceptor site of the CD7 gene sequence.

In one aspect, provided herein is a population of modified immune cells, wherein a plurality of the population of cells comprise a single target nucleobase modification in each one of at least four gene sequences or regulatory elements thereof, and wherein the plurality of the population of cells having the modification exhibit reduced immunogenicity or increased anti-neoplasia activity as compared to a plurality of control cells of a same type without the modification.

In some embodiments, the plurality of cells comprises at least 50% of the population.

In some embodiments, each one of the at least four gene sequences is a checkpoint inhibitor gene sequence, an immune response regulation gene sequence, or an immunogenic gene sequence.

In some embodiments, the at least four gene sequences comprise a TCR component gene sequence, a check point inhibitor gene sequence, or a T cell marker gene sequence.

In some embodiments, the at least four gene sequences comprise a TRAC gene sequence.

In some embodiments, the at least four gene sequences comprise a PDCD1 gene sequence.

In some embodiments, the at least four gene sequences comprise CD52 gene sequence.

In some embodiments, the at least four gene sequences comprises a CD7 gene sequence.

In the population of some embodiments, expression of at least one of the at least four genes is reduced by at least 80% in the plurality of cells having the modification as compared to a control cell without the modification

In the population of some embodiments, expression of each one of the at least four genes is reduced by at least 80% in the plurality of cells having the modification as compared to a control cell without the modification.

In some embodiments, the plurality of the population comprises a modification at a single target nucleobase in each one of five gene sequences or regulatory elements thereof, wherein each one of the five gene sequences is a checkpoint inhibitor gene sequence, an immune response regulation gene sequence, or an immunogenic gene sequence.

In some embodiments, the plurality of the population comprises a modification at a single target nucleobase in each one of six gene sequences or regulatory elements thereof, wherein each one of the six sequences is a checkpoint inhibitor gene sequence, an immune response regulation gene sequence, or an immunogenic gene sequence

In some embodiments, the plurality of the population comprises a modification at a single target nucleobase in each one of seven gene sequences or regulatory elements thereof, wherein each one of the seven gene sequences is a checkpoint inhibitor gene sequence, an immune response regulation gene sequence, or an immunogenic gene sequence.

In some embodiments, the plurality of the population comprises a modification at a single target nucleobase in each one of eight gene sequences or regulatory elements thereof, wherein each one of the eight gene sequences is a checkpoint inhibitor gene sequence, an immune response regulation gene sequence, or an immunogenic gene sequence.

In the population of some embodiments, the expression of at least one of the five, six, seven, or eight genes is reduced by at least 90% in the plurality of cells having the modification as compared to a control cell without the modification.

In the population of some embodiments, the expression of each one of the five, six, seven, or eight genes is reduced by at least 90% in the plurality of cells having the modification as compared to a control cell without the modification.

In the population of some embodiments, the expression of at least one of the five, six, seven, or eight genes is reduced by at least 90% in the plurality of cells having the modification as compared to a control cell without the modification.

In some embodiments, the expression of each one of the five, six, seven, or eight genes is reduced by at least 90% in the plurality of cells having the modification as compared to a control cell without the modification.

In some embodiments, the five, six, seven, or eight gene sequences or regulatory elements thereof are selected from the group consisting of a CD2 gene sequence, a TRAC gene sequence, a CD3 epsilon gene sequence, a CD3 gamma gene sequence, a CD3 delta gene sequence, a TRBC1 gene sequence, a TRBC2 gene sequence, a CD4 gene sequence, a CD5 gene sequence, a CD7 gene sequence, a CD30 gene sequence, a CD33 gene sequence, a CD52 gene sequence, a CD70 gene sequence, a B2M gene sequence, and a CIITA gene sequence.

In one aspect, provided herein is a population of modified immune cells, wherein a plurality of the population comprise a single target nucleobase modification in each one of a TRAC gene sequence, a PDCD1 gene sequence, a CD52 gene sequence, and a CD7 gene sequence, and wherein the plurality of the population having the modification exhibit reduced immunogenicity or increased anti-neoplasia activity as compared to a plurality of control cells of a same type without the modification.

In some embodiments, the plurality of the population further comprises a single target nucleobase modification in a CD2 gene sequence, a CD5 gene sequence, a CIITA gene sequence, a B2M gene sequence, or a regulatory element of each thereof. In some embodiments, the plurality of the population further comprises a single target nucleobase modification in a gene sequence of a gene selected from the group consisting of a CD2 gene sequence, a TRAC gene sequence, a CD3 epsilon gene sequence, a CD3 gamma gene sequence, a CD3 delta gene sequence, a TRBC1 gene sequence, a TRBC2 gene sequence, a CD4 gene sequence, a CD5 gene sequence, a CD7 gene sequence, a CD30 gene sequence, a CD33 gene sequence, a CD52 gene sequence, a CD70 gene sequence, a B2M gene sequence, and a CIITA gene sequence or a regulatory element of each thereof. In some embodiments, the plurality of the population comprises a single nucleobase modification in each one of a TRAC gene sequence, a PDCD1 gene sequence, a CD52 gene sequence, a CD7 gene sequence, a CD2 gene sequence, a CD5 gene sequence, a CIITA gene sequence, and a B2M gene sequence.

In the population of modified immune cells of some embodiments, the plurality of the population comprises no detectable translocation.

In the population of modified immune cells of some embodiments, the at least 60% of the population of immune cells are viable. In the population of modified immune cells of some embodiments, the at least 60% of the population of immune cells expand at least 80% of expansion rate of a population of control cells of a same type without the modification. In the population of modified immune cells of some embodiments, the population of immune cells are human cells. In the population of modified immune cells of some embodiments, the population of immune cells are cytotoxic T cells, regulatory T cells, T helper cells, dendritic cells, B cells, or NK cells. In the population of modified immune cells of some embodiments, the population of immune cells are derived from a single human donor. In the population of modified immune cells of some embodiments, the plurality of cells having the modification further comprises a polynucleotide that encodes an exogenous functional chimeric antigen receptor (CAR) or a functional fragment thereof.

In some embodiments, the at least 50% of the population of immune cells express the CAR.

In some embodiments, the the CAR specifically binds a marker associated with neoplasia.

In some embodiments, the neoplasia is a T cell cancer, a B cell cancer, a lymphoma, a leukemia, or a multiple myeloma.

In some embodiments, the CAR specifically binds CD7.

In some embodiments, the CAR specifically binds BCMA.

In some embodiments, the single target nucleobase is in an exon.

In some embodiments, the single target nucleobase is within an exon 1, an exon 2, or an exon 3 of the TRAC gene sequence.

In some embodiments, the single target nucleobase is within an exon 1, an exon 2, or an exon 5 of the PCDC1 gene sequence.

In some embodiments, the single target nucleobase is within an exon 1 or an exon 2 of the CD52 gene sequence.

In some embodiments, the single target nucleobase is within an exon 1, an exon 2, or an exon 3 of a CD7 gene sequence.

In the population of modified immune cells of some embodiments, the single target nucleobase is in a splice donor site or a splice acceptor site.

In some embodiments, the single target nucleobase is in an exon 1 splice acceptor site, an exon 1 splice donor site, or an exon 3 splice acceptor site of the TRAC gene sequence.

In some embodiments, the single target nucleobase is in an exon 1 splice acceptor site, an exon 1 splice donor site, an exon 2 splice acceptor site, an exon 3 splice donor site, an exon 4 splice acceptor site, an exon 4 splice donor site, or an exon 5 splice acceptor site of the PDCD1 gene sequence.

In some embodiments, the single target nucleobase is in an exon 1 splice donor site, or an exon 2 splice acceptor site of the CD52 gene sequence.

In some embodiments, the single target nucleobase is in an exon 1 splice donor site, an exon 2 splice donor site, an exon 2 splice acceptor site, or an exon 3 splice acceptor site of the CD7 gene sequence.

In one aspect, provided herein is a composition comprising deaminase and a nucleic acid sequence, wherein the guide nucleic acid sequence comprises a sequence selected from the group consisting of UUCGUAUCUGUAAAACCAAG, CCUACCUGUCACCAGGACCA, CUCUUACCUGUACCAUAACC, CACCUACCUAAGAACCAUCC, ACUCACGCUGGAUAGCCUCC, ACUCACCCAGCAUCCCCAGC, CACUCACCUUAGCCUGAGCA, and CACGCACCUGGACAGCUGAC.

In some embodiments, the deaminase is associated with a nucleic acid programmable DNA binding protein (napDNAbp) to form a base editor.

In some embodiments, the napDNAbp comprises a Cas9 nickase or nuclease dead Cas9 and wherein the deaminase is a cytidine deaminase.

In some embodiments, the base editor further comprises a uracil glycosylase inhibitor.

In some embodiments, the napDNAbp comprises a Cas9 nickase or nuclease dead Cas9 and wherein the deaminase is a adenosine deaminase.

In one aspect, provided herein is a composition comprising a polymerase and a guide nucleic acid sequence, wherein the guide nucleic acid sequence comprises a sequence selected from the group consisting of the group consisting of UUCGUAUCUGUAAAACCAAG, CCUACCUGUCACCAGGACCA, CUCUUACCUGUACCAUAACC, CACCUACCUAAGAACCAUCC, ACUCACGCUGGAUAGCCUCC, ACUCACCCAGCAUCCCCAGC, CACUCACCUUAGCCUGAGCA, and CACGCACCUGGACAGCUGAC.

In some embodiments, the polymerase is a reverse transcriptase and wherein the guide nucleic acid sequence is an extended guide nucleic acid sequence comprising a reverse transcription template sequence, a reverse transcription primer binding site, or a combination thereof.

In one aspect, provided herein is a method for producing a modified immune cell with reduced immunogenicity and/or increased anti-neoplasia activity, the method comprising: a) modifying a single target nucleobase in a first gene sequence or a regulatory element thereof in an immune cell; and b) modifying a second gene sequence or a regulatory element thereof in the immune cell with a Cas12 polypeptide, wherein the Cas12 polypeptide generates a site-specific cleavage in the second gene sequence; wherein each of the first gene and the second gene is a immunogenic gene, a checkpoint inhibitor gene, or an immune response regulation gene, thereby generating a modified immune cell with reduced immunogenicity and/or increased anti-neoplasia activity.

In some embodiments, the method further comprises expressing an exogenous functional chimeric antigen receptor (CAR) or a functional fragment thereof in the immune cell.

In some embodiments, a polynucleotide encoding the CAR or the functional fragment thereof is inserted into the site specific cleavage generated by the Cas12 polypeptide.

In some embodiments, the Cas12 polypeptide is a Cas12b polypeptide.

In one aspect, provided herein is a method for producing a modified immune cell with reduced immunogenicity and/or increased anti-neoplasia activity, the method comprising:

a) modifying a single target nucleobase in a first gene sequence or a regulatory element thereof in an immune cell; and b) modifying a second gene sequence or a regulatory element thereof in the immune cell by inserting an exogenous functional chimeric antigen receptor (CAR) or a functional fragment thereof or an exogenous functional T cell receptor or a functional fragment thereof in the second gene; wherein each of the first gene and the second gene is a immunogenic gene, a checkpoint inhibitor gene, or an immune response regulation gene, thereby generating a modified immune cell with reduced immunogenicity and/or increased anti-neoplasia activity.

In some embodiments, the step b) further comprises generating a site-specific cleavage in the second gene sequence with a nucleic acid programmable DNA binding protein (napDNAbp).

In some embodiments, the napDNAbp is a Cas12b.

In some embodiments, the expression of the first gene is reduced by at least 60% or wherein expression of the second gene is reduced by at least 60% as compared to a control cell of a same type without the modification.

In some embodiments, the first gene is selected from the group consisting of CD3 epsilon, CD3 gamma, CD3 delta, CD4, TRAC, TRBC1, TRBC2, PDCD1, CD30, CD33, CD7, CD52, B2M, CD70, CIITA, CD2, and CD5.

In some embodiments, the first gene or the second gene is selected from the group consisting of TRAC, CIITA, CD2, CD5, CD7, and CD52.

In some embodiments, the second gene is TRAC.

In some embodiments, the step a) further comprises modifying a single target nucleobase in two other gene sequences or regulatory elements thereof.

In some embodiments, the step a) further comprises modifying a single target nucleobase in three other gene sequences or regulatory elements thereof.

In some embodiments, the step a) further comprises modifying a single target nucleobase in four other gene sequences or regulatory elements thereof.

In some embodiments, the step a) further comprises modifying a single target nucleobase in five other gene sequences or regulatory elements thereof.

In some embodiments, the step a) further comprises modifying a single target nucleobase in six other gene sequences or regulatory elements thereof.

In some embodiments, the step a) further comprises modifying a single target nucleobase in seven other gene sequences or regulatory elements thereof.

In some embodiments, the modifying in step a) comprises deaminating the single target nucleobase with a base editor comprising a deaminase and a nucleic acid programmable DNA binding protein (napDNAbp).

In some embodiments, the napDNAbp comprises a Cas9 nickase or nuclease dead Cas9.

In some embodiments, the deaminase is a cytidine deaminase and wherein the modification comprises conversion of a cytidine (C) to a thymine (T).

In some embodiments, the deaminase is an adenosine deaminase and wherein the modification comprises conversion of an adenine (A) to a guanine (G).

In some embodiments, the modifying in a) comprises contacting the immune cell with a guide nucleic acid sequence.

In some embodiments, the guide nucleic acid sequence comprises a sequence selected from the group consisting of UUCGUAUCUGUAAAACCAAG, CCUACCUGUCACCAGGACCA, CUCUUACCUGUACCAUAACC, CACCUACCUAAGAACCAUCC, ACUCACGCUGGAUAGCCUCC, ACUCACCCAGCAUCCCCAGC, CACUCACCUUAGCCUGAGCA, and CACGCACCUGGACAGCUGAC.

In some embodiments, the modifying in b) comprises contacting the immune cell with a guide nucleic acid sequence.

In some embodiments, the guide nucleic acid sequence comprises a sequence selected from sequences in Table 1.

In some embodiments, the modifying in a) comprises replacing the single target nucleobase with a different nucleobase by target-primed reverse transcription with a reverse transcriptase and an extended guide nucleic acid sequence, wherein the extended guide nucleic acid sequence comprises a reverse transcription template sequence, a reverse transcription primer binding site, or a combination thereof.

In some embodiments, wherein the modifying in a) and b) generates less than 1% indels in the immune cell.

In some embodiments, the modifying in a) and b) generates less than 5% off target modification in the immune cell.

In some embodiments, the modifying in a) and b) generate less than 5% non-target modification in the immune cell.

In some embodiments, the immune cell is a human cell.

In some embodiments, the immune cell is a cytotoxic T cell, a regulatory T cell, a T helper cell, a dendritic cell, a B cell, or a NK cell.

In some embodiments, the CAR specifically binds a marker associated with neoplasia.

In some embodiments, the CAR specifically binds CD7.

In one aspect, provided herein is a modified immune cell with reduced immunogenicity and/or increased anti-neoplasia activity, wherein the modified immune cell comprises:

a) a single target nucleobase modification in a first gene sequence or a regulatory element thereof; and b) a modification in a second gene sequence or a regulatory element thereof, wherein the modification is a Cas12 polypeptide generated site-specific cleavage; wherein each of the first gene and the second gene is a immunogenic gene, a checkpoint inhibitor gene, or an immune response regulation gene. In one embodiment, the immune cell further comprises an exogenous functional chimeric antigen receptor (CAR) or a functional fragment thereof.

In some embodiments, a polynucleotide encoding the CAR or the functional fragment thereof is inserted into the site specific cleavage generated by the Cas12 polypeptide.

In one aspect, provided herein is a modified immune cell with reduced immunogenicity and/or increased anti-neoplasia activity, the modified immune cell comprising: a) a single target nucleobase modification in a first gene sequence or a regulatory element thereof in an immune cell; and b) a modification in a second gene sequence or a regulatory element thereof, wherein the modification is an insertion of an exogenous chimeric antigen receptor (CAR) or a functional fragment thereof or an exogenous T cell receptor or a functional fragment thereof; wherein each of the first gene and the second gene is a immunogenic gene, a checkpoint inhibitor gene, or immune response regulation gene.

In some embodiments, the modification in b) is generated by a site-specific cleavage with a Cas12b.

In some embodiments, expression of the first gene is reduced by at least 60% or wherein expression of the second gene is reduced by at least 60% as compared to a control cell of a same type without the modification.

In some embodiments, the first gene or the second gene is selected from the group consisting of CD3 epsilon, CD3 gamma, CD3 delta, CD4, TRAC, TRBC1, TRBC2, PDCD1, CD30, CD33, CD7, CD52, B2M, CD70, CIITA, CD2, and CD5.

In some embodiments, the first gene or the second gene is selected from the group consisting of TRAC, CD2, CD5, CD7, and CD52.

In some embodiments, the second gene is TRAC.

In some embodiments, the immune cell further comprises modification in a single target nucleobase in two other gene sequences or regulatory elements thereof.

In some embodiments, the immune cell further comprises modification in a single target nucleobase in three other gene sequences or regulatory elements thereof.

In some embodiments, the immune cell further comprises modification in a single target nucleobase in four other gene sequences or regulatory elements thereof.

In some embodiments, the immune cell further comprises modification in a single target nucleobase in five other gene sequences or regulatory elements thereof.

In some embodiments, the immune cell further comprises modification in a single target nucleobase in six other gene sequences or regulatory elements thereof.

In some embodiments, the immune cell further comprises modification in a single target nucleobase in seven other gene sequences or regulatory elements thereof.

In some embodiments, the modification in a) is generated by a base editor comprising a deaminase and a nucleic acid programmable DNA binding protein (napDNAbp).

In some embodiments, the deaminase is a cytidine deaminase and the modification comprises conversion of a cytidine (C) to a thymine (T).

In some embodiments, the deaminase is an adenosine deaminase and wherein the modification comprises conversion of an adenine (A) to a guanine (G).

In some embodiments, the immune cell comprises less than 1% indels in the genome.

In some embodiments, the immune cell is a human cell.

In some embodiments, the immune cell is a cytotoxic T cell, a regulatory T cell, a T helper cell, a dendritic cell, a B cell, or a NK cell.

In some embodiments, the CAR specifically binds a marker associated with neoplasia.

In some embodiments, the CAR specifically binds CD7.

In some embodiments, the modification in b) is an insertion in exon 1 in the TRAC gene sequence.

In one aspect, provided herein is a population of modified immune cells, wherein a plurality of the population of immune cells comprises: a) a single target nucleobase modification in a first gene sequence or a regulatory element thereof in an immune cell; and b) a modification in a second gene sequence or a regulatory element thereof, wherein the modification is a Cas12 polypeptide generated site-specific cleavage; wherein each of the first gene and the second gene is a immunogenic gene, a checkpoint inhibitor gene, or an immune response regulation gene, and wherein the plurality of the population comprises an exogenous chimeric antigen receptor (CAR) or a functional fragment thereof.

In some embodiments, a polynucleotide encoding the CAR or the functional fragment thereof is inserted into the site specific cleavage generated by the Cas12 polypeptide.

In one aspect, provided herein is a population of modified immune cells, wherein a plurality of the population of immune cells comprises: a) a single target nucleobase modification in a first gene sequence or a regulatory element thereof; and b) a modification in a second gene sequence or a regulatory sequence thereof, wherein the modification is an insertion of an exogenous chimeric antigen receptor (CAR) or a functional fragment thereof or an exogenous T cell receptor or a functional fragment thereof; wherein each of the first gene and the second gene is a immunogenic gene, a checkpoint inhibitor gene, or immune response regulation gene, and wherein the plurality of cells with the modification in a) or b) exhibit reduced immunogenicity and/or increased anti-neoplasia activity. In some embodiments, the modification in b) is generated by a site-specific cleavage with a Cas12b. In some embodiments, expression of the first gene is reduced by at least 60% or wherein expression of the second gene is reduced by at least 60% in the plurality of cells with the modification in a) or b) as compared to plurality of control cells of a same type without the modification.

In some embodiments, the first gene or the second gene is selected from the group consisting of CD3 epsilon, CD3 gamma, CD3 delta, CD4, TRAC, TRBC1, TRBC2, PDCD1, CD30, CD33, CD7, CD52, B2M, CD70, CIITA, CD2, and CD5.

In some embodiments, the first gene or the second gene is selected from the group consisting of TRAC, CIITA, CD2, CD5, CD7, and CD52.

In some embodiments, the first gene is TRAC, CD7, or CD52.

In some embodiments, the second gene is TRAC.

In some embodiments, the plurality of cells with the modification in a) or b) further comprises a modification in a single target nucleobase in two other gene sequences or regulatory elements thereof.

In some embodiments, the plurality of cells with the modification in a) or b) further comprises a single target nucleobase in three, four, five, or six other gene sequences or regulatory elements thereof.

In some embodiments, the modification in a) is generated by a base editor comprising a deaminase and a nucleic acid programmable DNA binding protein (napDNAbp) to form a base editor.

In some embodiments, the deaminase is a cytidine deaminase and wherein the modification comprises conversion of a cytidine (C) to a thymine (T).

In some embodiments, the deaminase is an adenosine deaminase and wherein the modification comprises conversion of an adenine (A) to a guanine (G).

In some embodiments, the base editor further comprises a uracil glycosylase inhibitor.

In some embodiments, at least 60% of the population of immune cells are viable.

In some embodiments, at least 60% of the population of immune cells expand at least 80% of expansion rate of a population of control cells of a same type without the modification.

In some embodiments, the population of modified immune cells have increased yield of modified immune cells compared to a reference population of cells. In some embodiments, the reference population is a population of immune cells modified with a Cas9 nuclease.

In some embodiments, the immune cells are a human cells.

In some embodiments, the immune cells is are cytotoxic T cells, regulatory T cells, T helper cells, dendritic cells, B cells, or NK cells.

In some embodiments, the CAR specifically binds a marker associated with neoplasia.

In some embodiments, the CAR specifically binds CD7.

In some embodiments, the modification in b) is an insertion in exon 1 in the TRAC gene sequence.

In one aspect, provided herein is a method for producing a modified immune cell with increased anti-neoplasia activity, the method comprising: modifying a single target nucleobase in a Cbl Proto Oncogene B (CBLB) gene sequence or a regulatory element thereof in an immune cell, wherein the modification reduces an activation threshold of the immune cell compared with an immune cell lacking the modification; thereby generating a modified immune cell with increased anti-neoplasia activity.

In one aspect, provided herein is a composition comprising a modified immune cell with increased anti-neoplasia activity, wherein the modified immune cell comprises: a modification in a single target nucleobase in a Cbl Proto-Oncogene B (CBLB) gene sequence or a regulatory element thereof, wherein the modified immune cell exhibits a reduced activation threshold compared with a control immune cell of a same type without the modification.

In one aspect, provided herein is a population of immune cells, wherein a plurality of the population of immune cells comprises: a modification in a single target nucleobase in a CBLB gene sequence or a regulatory element thereof, wherein the plurality of the population of the immune cells comprising the modification exhibit a reduced activation threshold compared with an control population of immune cells of a same type without the modification.

In one aspect, provided herein is a method for producing a population of modified immune cells with increased anti-neoplasia activity, the method comprising: modifying a single target nucleobase in a Cbl Proto Oncogene B (CBLB) gene sequence or a regulatory element thereof in a population of immune cells, wherein at least 50% of the population of immune cells are modified to comprise the single target nucleobase modification.

In one aspect, provided herein is a composition comprising at least four different guide nucleic acid sequences for base editing. In some embodiments, the composition further comprising a polynucleotide encoding a base editor polypeptide, wherein the base editor polypeptide comprises a nucleic acid programmable DNA binding protein (napDNAbp) and a deaminase. In some embodiments, the polynucleotide encoding the base editor is a mRNA sequence.

In some embodiments, the deaminase is a cytidine deaminase or an adenosine deaminase.

In some embodiments, the composition further comprises a base editor polypeptide, wherein the base editor polypeptide comprises a nucleic acid programmable DNA binding protein (napDNAbp) and a deaminase.

In some embodiments, the deaminase is a cytidine deaminase or an adenosine deaminase.

In some embodiments, the composition further comprises a lipid nanoparticle.

In some embodiments, the at least four guide nucleic acid sequences each hybridize with a gene sequence selected from the group consisting of CD2, CD3 epsilon, CD3 gamma, CD3 delta, CD4, CD5, CD7, CD30, CD33, CD52, CD70, and CIITA. In some embodiments, the at least 1, 2, 3, 4, 5, 6, 7, 8, or more genes or regulatory elements thereof are selected from CD2, CD3 epsilon, CD3 gamma, CD3 delta, CD4, CD5, CD7, CD30, CD33, CD52, CD70, and CIITA.

In some embodiments, the at least 1, 2, 3, 4, 5, 6, 7, 8, or more genes or regulatory elements thereof comprise one or more genes selected from CD2, CD3 epsilon, CD3 gamma, CD3 delta, CD4, CD5, CD7, CD30, CD33, CD52, CD70, and CIITA. In some embodiments, the at least 1, 2, 3, 4, 5, 6, 7, 8, or more genes or regulatory elements thereof are selected from ACAT1, ACLY, ADORA2A, AXL, B2M, BATF, BCL2L11, BTLA, CAMK2D, cAMP, CASP8, Cblb, CCR5, CD2, CD3D, CD3E, CD3G, CD4, CD5, CD7, CD8A, CD33, CD38, CD52, CD70, CD82, CD86, CD96, CD123, CD160, CD244, CD276, CDK8, CDKN1B, Chi311, CIITA, CISH, CSF2CSK, CTLA-4, CUL3, Cyp11a1, DCK, DGKA, DGKZ, DHX37, ELOB (TCEB2), ENTPD1 (CD39), FADD, FAS, GATA3, IL6, IL6R, IL10, IL10RA, IRF4, IRF8, JUNB, Lag3, LAIR-1 (CD305), LDHA, LIF, LYN, MAP4K4, MAPK14, MCJ, MEF2D, MGAT5, NR4A1, NR4A2, NR4A3, NT5E (CD73), ODC1, OTULINL (FAM105A), PAG1, PDCD1, PDIA3, PHD1 (EGLN2), PHD2 (EGLN1), PHD3 (EGLN3), PIK3CD, PIKFYVE, PPARa, PPARd, PRDMI1, PRKACA, PTEN, PTPN2, PTPN6, PTPN11, PVRIG (CD112R), RASA2, RFXANK, SELPG/PSGL1, SIGLEC1S, SLA, SLAMF7, SOCS1, Spry1, Spry2, STK4, SUV39, H1TET2, TGFbRII, TIGIT, Tim-3, TMEM222, TNFAIP3, TNFRSF8 (CD30), TNFRSF10B, TOX, TOX2, TRAC, TRBC1, TRBC2, UBASH3A, VHL, VISTA, In some embodiments, the at least four guide nucleic acid sequences each hybridize with a gene sequence selected from the group consisting of CD3epsilon, CD3 delta, CD3 gamma, TRAC, TRBC1, and TRBC2, CD2, CD5, CD7, CD52, CD70, and CIITA.

In some embodiments, the at least four guide nucleic acid sequences comprise a sequence selected from the group consisting of UUCGUAUCUGUAAAACCAAG, CCUACCUGUCACCAGGACCA, CUCUUACCUGUACCAUAACC, CACCUACCUAAGAACCAUCC, ACUCACGCUGGAUAGCCUCC, ACUCACCCAGCAUCCCCAGC, CACUCACCUUAGCCUGAGCA, and CACGCACCUGGACAGCUGAC.

In one aspect, provided herein is an immune cell comprising the composition of some of the embodiments described above, wherein the composition is introduced into the immune cell with electroporation.

In one aspect, provided herein is an immune cell comprising the composition of some of the embodiments described above, wherein the composition is introduced into the immune cell with electroporation, nucleofection, viral transduction, or a combination thereof.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “adenosine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxyadenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA (ecTadA) deaminase or a fragment thereof.

For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. In some embodiments, the TadA deaminase is an N-terminal truncated TadA. In particular embodiments, the TadA is any one of the TadAs described in PCT/US2017/045381, which is incorporated herein by reference in its entirety.

In certain embodiments, the adenosine deaminase comprises the amino acid sequence:

MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD,

which is termed “the TadA reference sequence.”

In some embodiments the TadA deaminase is a full-length E. coli TadA deaminase. For example, in certain embodiments, the adenosine deaminase comprises the amino acid sequence:

MRRAFITGVFFLSEVEFSHEYWMRHALTLAKRAWDEREVPVGAVLVHNNR
VIGEGWNRPIGRHDPTAHAEIMALRQGGLVMQNYRLIDATLYVTLEPCVM
CAGAMIHSRIGRVVFGARDAKTGAAGSLMDVLHHPGMNHRVEITEGILAD
ECAALLSDFFRMRRQEIKAQKKAQSSTD

It should be appreciated, however, that additional adenosine deaminases useful in the present application would be apparent to the skilled artisan and are within the scope of this disclosure. For example, the adenosine deaminase may be a homolog of adenosine deaminase acting on tRNA (AD AT). Exemplary AD AT homologs include, without limitation:

Staphylococcus aureus TadA:
MGSHMTNDIYFMTLAIEEAKKAAQLGEVPIGAIITKDDEVIARAHNLRET
LQQPTAHAEHIAIERAAKVLGSWRLEGCTLYVTLEPCVMCAGTIVMSRIP
RVVYGADDPKGGCSGS LMNLLQQS NFNHRAIVDKG VLKE AC S TL
LTTFFKNLRANKKS TN
Bacillus subtilis TadA:
MTQDELYMKEAIKEAKKAEEKGEVPIGAVLVINGEIIARAHNLRETEQRS
IAHAEMLVIDEACKALGTWRLEGATLYVTLEPCPMCAGAVVLSRVEKVVF
GAFDPKGGC SGTLMN LLQEERFNHQAEVVSGVLEEECGGMLSAFFREL
RKKKKAARKNLSE
Salmonella typhimurium (S. typhimurium) TadA:
MPPAFITGVTSLSDVELDHEYWMRHALTLAKRAWDEREVPVGAVLVHNHR
VIGEGWNRPIGRHDPTAHAEIMALRQGGLVLQNYRLLDTTLYVTLEPCVM
CAGAMVHSRIGRVVFGARDAKTGAAGSLIDVLHHPGMNHRVEIIEGVLRD
ECATLLSDFFRMRRQEIKALKKADRAEGAGPAV
Shewanella putrefaciens (S. putrefaciens) TadA:
MDE YWMQVAMQM AEKAEAAGE VPVGA VLVKDGQQIATGYNLS IS
QHDPT AHAEILCLRSAGKKLENYRLLDATLYITLEPCAMCAGAMVHSR
IARVVYGARDEKTGAAGTVVNLLQHPAFNHQVEVTSGVLAEACSAQLSR
FFKRRRDEKKALKLAQRAQQGIE
Haemophilus influenzae F3031 (H. influenzae) TadA:
MDAAKVRSEFDEKMMRYALELADKAEALGEIPVGAVLVDDARNIIGEGWN
LSIVQSDPT AH AEIIALRNG AKNIQN YRLLNS TLY VTLEPCTMC
AG AILHS RIKRLVFG AS DYK TGAIGSRFHFFDDYKMNHTLEITSG
VLAEECSQKLSTFFQKRREEKKIEKALLKSLSDK
Caulobacter crescentus (C. crescentus) TadA:
MRTDESEDQDHRMMRLALDAARAAAEAGETPVGAVILDPSTGEVIATAGN
GPIAAHDPTAHAEIAAMRAAAAKLGNYRLTDLTLVVTLEPCAMCAGAISH
ARIGRVVFGADDPKGGAVVHGPKFFAQPTCHWRPEVTGGVLADESADLLR
GFFRARRKAKI
Geobacter sulfurreducens (G. sulfurreducens) TadA:
MSSLKKTPIRDDAYWMGKAIREAAKAAARDEVPIGAVIVRDGAVIGRGHN
LREGSNDPSAHAEMIAIRQAARRSANWRLTGATLYVTLEPCLMCMGAIIL
ARLERVVFGCYDPKGGAAGSLYDLSADPRLNHQVRLSPGVCQEECGTMLS
DFFRDLRRRKKAKATPALFIDERKVPPEP
TadA7.10
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD

By “agent” is meant any small molecule chemical compound, antibody, nucleic acid molecule, or polypeptide, or fragments thereof.

By “alteration” is meant a change in the structure, expression levels or activity of a gene or polypeptide as detected by standard art known methods such as those described herein. As used herein, an alteration (e.g., increase or decrease) includes a 10% change in expression levels, a 25% change, a 40% change, and a 50% or greater change in expression levels.

“Allogeneic,” as used herein, refers to cells of the same species that differ genetically to the cell in comparison.

By “analog” is meant a molecule that is not identical, but has analogous functional or structural features. For example, a polypeptide analog retains the biological activity of a corresponding naturally-occurring polypeptide, while having certain sequence modifications that enhance the analog's function relative to a naturally occurring polypeptide. Such modifications could increase the analog's protease resistance, membrane permeability, or half-life, without altering, for example, polynucleotide binding activity. In another example, a polynucleotide analog retains the biological activity of a corresponding naturally-occurring polynucleotide while having certain modifications that enhance the analog's function relative to a naturally occurring polynucleotide. Such modifications could increase the polynucleotide's affinity for DNA, half-life, and/or nuclease resistance, an analog may include an unnatural nucleotide or amino acid.

By “anti-neoplasia activity” is meant preventing or inhibiting the maturation and/or proliferation of neoplasms.

“Autologous,” as used herein, refers to cells from the same subject.

By “B cell maturation antigen, or tumor necrosis factor receptor superfamily member 17 polypeptide, (BCMA)” is meant a protein having at least about 85% amino acid sequence identify to NCBI Accession No. NP_001183 or a fragment thereof that is expressed on mature B lymphocytes. An exemplary BCMA polypeptide sequence is provided below.

>NP_001183.2 tumor necrosis factor receptor superfamily member 17 [Homo sapiens]

MLQMAGQCSQNEYFDSLLHACIPCQLRCSSNTPPLTCQRYCNASVTNSVK
GTNAILWTCLGLSLIISLAVFVLMFLLRKINSEPLKDEFKNTGSGLLGMA
NIDLEKSRTGDEIILPRGLEYTVEECTCEDCIKSKPKVDSDHCFPLPAME
EGATILVTTKTNDYCKSLPAALSATEIEKSISAR

This antigen can be targeted in relapsed or refractory multiple myeloma and other hematological neoplasia therapies.

By “B cell maturation antigen, or tumor necrosis factor receptor superfamily member 17, (BCMA) polynucleotide” is meant a nucleic acid molecule encoding a BCMA polypeptide. The BCMA gene encodes a cell surface receptor that recognizes B cell activating factor. An exemplary B2M polynucleotide sequence is provided below.

>NM_001192.2 Homo sapiens TNF receptor superfamily member 17 (TNFRSF17), mRNA

AAGACTCAAACTTAGAAACTTGAATTAGATGTGGTATTCAAATCCTTAGC
TGCCGCGAAGACACAGACAGCCCCCGTAAGAACCCACGAAGCAGGCGAAG
TTCATTGTTCTCAACATTCTAGCTGCTCTTGCTGCATTTGCTCTGGAATT
CTTGTAGAGATATTACTTGTCCTTCCAGGCTGTTCTTTCTGTAGCTCCCT
TGTTTTCTTTTTGTGATCATGTTGCAGATGGCTGGGCAGTGCTCCCAAAA
TGAATATTTTGACAGTTTGTTGCATGCTTGCATACCTTGTCAACTTCGAT
GTTCTTCTAATACTCCTCCTCTAACATGTCAGCGTTATTGTAATGCAAGT
GTGACCAATTCAGTGAAAGGAACGAATGCGATTCTCTGGACCTGTTTGGG
ACTGAGCTTAATAATTTCTTTGGCAGTTTTCGTGCTAATGTTTTTGCTAA
GGAAGATAAACTCTGAACCATTAAAGGACGAGTTTAAAAACACAGGATCA
GGTCTCCTGGGCATGGCTAACATTGACCTGGAAAAGAGCAGGACTGGTGA
TGAAATTATTCTTCCGAGAGGCCTCGAGTACACGGTGGAAGAATGCACCT
GTGAAGACTGCATCAAGAGCAAACCGAAGGTCGACTCTGACCATTGCTTT
CCACTCCCAGCTATGGAGGAAGGCGCAACCATTCTTGTCACCACGAAAAC
GAATGACTATTGCAAGAGCCTGCCAGCTGCTTTGAGTGCTACGGAGATAG
AGAAATCAATTTCTGCTAGGTAATTAACCATTTCGACTCGAGCAGTGCCA
CTTTAAAAATCTTTTGTCAGAATAGATGATGTGTCAGATCTCTTTAGGAT
GACTGTATTTTTCAGTTGCCGATACAGCTTTTTGTCCTCTAACTGTGGAA
ACTCTTTATGTTAGATATATTTCTCTAGGTTACTGTTGGGAGCTTAATGG
TAGAAACTTCCTTGGTTTCATGATTAAACTCTTTTTTTTCCTGA

By “base editor (BE),” or “nucleobase editor (NBE)” is meant an agent that binds a polynucleotide and has nucleobase modifying activity. In one embodiment, the agent binds the polynucleotide at a specific sequence using a nucleic acid programmable DNA binding protein. In another embodiment, the base editor is an enzyme capable of modifying a cytidine base within a nucleic acid molecule (e.g., DNA). In some embodiments, the base editor is capable of deaminating a base within a nucleic acid molecule. In some embodiments, the base editor is capable of deaminating a base within a DNA molecule. In some embodiments, the base editor is capable of deaminating a cytidine in DNA. In some embodiments, the base editor is a fusion protein comprising a cytidine deaminase or an adenosine deaminase. In some embodiments, the base editor is a Cas9 protein fused to a cytidine deaminase or an adenosine deaminase. In some embodiments, the base editor is a Cas9 nickase (nCas9) fused to a cytidine deaminase or an adenosine deaminase. In some embodiments, the base editor is fused to an inhibitor of base excision repair, for example, a UGI domain. In some embodiments, the fusion protein comprises a Cas9 nickase fused to a deaminase and an inhibitor of base excision repair, such as a UGI domain. In some embodiments, the cytidine deaminase or an or an adenosine deaminase nucleobase editor polypeptide comprising the following domains A-B:

NH₂-[A-B]—COOH,

wherein A comprises a cytidine deaminase domain, an adenosine deaminase domain or an active fragment thereof, and wherein B comprises one or more domains having nucleic acid sequence specific binding activity. In one embodiment, the cytidine or adenosine deaminase Nucleobase Editor polypeptide of the previous aspect contains:

NH₂-[Aₙ-Bₒ]—COOH,

wherein A comprises: a cytidine deaminase domain, an adenosine deaminase domain, or an active fragment thereof, wherein n is an integer: 1, 2, 3, 4, or 5; and wherein B comprises a domain having nucleic acid sequence specific binding activity; and wherein o is an integer: 1, 2, 3, 4, or 5. In one embodiment, the polypeptide contains one or more nuclear localization sequences. In one embodiment, the polypeptide contains at least one of said nuclear localization sequences is at the N-terminus or C-terminus. In one embodiment, the polypeptide contains the nuclear localization signal is a bipartite nuclear localization signal. In one embodiment, the polypeptide contains one or more domains linked by a linker.

In some embodiments, the base editor is a cytidine base editor (CBE). In some embodiments, the base editor is an adenosine base editor (ABE). In some embodiments, the base editor is an adenosine base editor (ABE) and a cytidine base editor (CBE). In some embodiments, the base editor is a nuclease-inactive Cas9 (dCas9) fused to an adenosine deaminase. In some embodiments, the Cas9 is a circular permutant Cas9 (e.g., spCas9 or saCas9). Circular permutant Cas9s are known in the art and described, for example, in Oakes et al., Cell 176, 254-267, 2019. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In other embodiments the base editor is an abasic base editor.

In some embodiments, an adenosine deaminase is evolved from TadA. In some embodiments, the polynucleotide programmable DNA binding domain is a CRISPR associated (e.g., Cas or Cpf1) enzyme. In some embodiments, the base editor is a catalytically dead Cas9 (dCas9) fused to a deaminase domain. In some embodiments, the base editor is a Cas9 nickase (nCas9) fused to a deaminase domain. In some embodiments, the base editor is fused to an inhibitor of base excision repair (BER). In some embodiments, the inhibitor of base excision repair is a uracil DNA glycosylase inhibitor (UGI). In some embodiments, the inhibitor of base excision repair is an inosine base excision repair inhibitor. Details of base editors are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A⋅T to G⋅C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference.

In some embodiments, base editors are generated by cloning an adenosine deaminase variant (e.g., TadA*7.10) into a scaffold that includes a circular permutant Cas9 (e.g., spCAS9) and a bipartite nuclear localization sequence. Circular permutant Cas9s are known in the art and described, for example, in Oakes et al., Cell 176, 254-267, 2019. Exemplary circular permutant sequences are set forth below, in which the bold sequence indicates sequence derived from Cas9, the italics sequence denotes a linker sequence, and the underlined sequence denotes a bipartite nuclear localization sequence.

CP5 (with MSP “NGC=Pam Variant with mutations Regular Cas9 likes NGG” PID=Protein Interacting Domain and “D10A” nickase):

EIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWD
PKKYGGFMQPTVAYSVLVVAKVEKGKSKKLKSVKELLGITIMERSSFEKN
PIDFLEAKGYKEVKKDLIIKLPKYSLFELENGRKRMLASAKFLQKGNELA
LPSKYVNFLYLASHYEKLKGSPEDNEQKQLFVEQHKHYLDEIIEQISEFS
KRVILADANLDKVLSAYNKHRDKPIREQAENIIHLFTLTNLGAPRAFKYF
DTTIARKEYRSTKEVLDATLIHQSITGLYETRIDLSQLGGDGGSGGSGGS
GGSGGSGGSGGMDKKYSIGLAIGTNSVGWAVITDEYKVPSKKFKVLGNTD
RHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEEL
LVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVD
HIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRM
NTKYDENDKLIREVKVITLKSKLVSDFRKDFQFYKVREINNYHHAHDAYL
NAVVGTALIKKYPKLESEFVYGDYKVYDVRKMIAKSEQEGADKRTADGSE
FESPKKKRKV*

The nucleobase components and the polynucleotide programmable nucleotide binding component of a base editor system may be associated with each other covalently or non-covalently. For example, in some embodiments, the deaminase domain can be targeted to a target nucleotide sequence by a polynucleotide programmable nucleotide binding domain. In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component, e.g., the deaminase component can comprise an additional heterologous portion or domain that is capable of interacting with, associating with, or capable of forming a complex with an additional heterologous portion or domain that is part of a polynucleotide programmable nucleotide binding domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a steril alpha motif, a telomerase Ku binding motif and Ku protein, a telomerase Sm7 binding motif and Sm7 protein, or a RNA recognition motif.

A base editor system may further comprise a guide polynucleotide component. It should be appreciated that components of the base editor system may be associated with each other via covalent bonds, noncovalent interactions, or any combination of associations and interactions thereof. In some embodiments, a deaminase domain can be targeted to a target nucleotide sequence by a guide polynucleotide. For example, in some embodiments, the nucleobase editing component of the base editor system, e.g., the deaminase component, can comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain (e.g., polynucleotide binding domain such as an RNA or DNA binding protein) can be fused or linked to the deaminase domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or a RNA recognition motif.

In some embodiments, a base editor system can further comprise an inhibitor of base excision repair (BER) component. It should be appreciated that components of the base editor system may be associated with each other via covalent bonds, noncovalent interactions, or any combination of associations and interactions thereof. The inhibitor of BER component may comprise a base excision repair inhibitor. In some embodiments, the inhibitor of base excision repair can be a uracil DNA glycosylase inhibitor (UGI). In some embodiments, the inhibitor of base excision repair can be an inosine base excision repair inhibitor. In some embodiments, the inhibitor of base excision repair can be targeted to the target nucleotide sequence by the polynucleotide programmable nucleotide binding domain. In some embodiments, a polynucleotide programmable nucleotide binding domain can be fused or linked to an inhibitor of base excision repair. In some embodiments, a polynucleotide programmable nucleotide binding domain can be fused or linked to a deaminase domain and an inhibitor of base excision repair. In some embodiments, a polynucleotide programmable nucleotide binding domain can target an inhibitor of base excision repair to a target nucleotide sequence by non-covalently interacting with or associating with the inhibitor of base excision repair. For example, in some embodiments, the inhibitor of base excision repair component can comprise an additional heterologous portion or domain that is capable of interacting with, associating with, or capable of forming a complex with an additional heterologous portion or domain that is part of a polynucleotide programmable nucleotide binding domain. In some embodiments, the inhibitor of base excision repair can be targeted to the target nucleotide sequence by the guide polynucleotide. For example, in some embodiments, the inhibitor of base excision repair can comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain of the guide polynucleotide (e.g., polynucleotide binding domain such as an RNA or DNA binding protein) can be fused or linked to the inhibitor of base excision repair. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or a RNA recognition motif. By “base editing activity” is meant acting to chemically alter a base within a polynucleotide. In one embodiment, a first base is converted to a second base. In one embodiment, the base editing activity is cytidine deaminase activity, e.g., converting target C⋅G to T⋅A. In another embodiment, the base editing activity is adenosine deaminase activity, e.g., converting A⋅T to G⋅C.

By “beta-2 microglobulin (B2M) polypeptide” is meant a protein having at least about 85% amino acid sequence identity to UniProt Accession No. P61769 or a fragment thereof and having immunomodulatory activity. An exemplary B2M polypeptide sequence is provided below.

>sp|P61769|B2MG_HUMAN Beta-2-microglobulin OS═Homo sapiens OX=9606 GN=B2M PE=1 SV=1

MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDM

By “beta-2-microglobulin (B2M) polynucleotide” is meant a nucleic acid molecule encoding a B2M polypeptide. The beta-2-microglobulin gene encodes a serum protein associated with the major histocompatibility complex. B2M is involved in non-self recognition by host CD8+ T cells. An exemplary B2M polynucleotide sequence is provided below.

>DQ217933.1 Homo sapiens beta-2-microglobin
(B2M) gene, complete cds
CATGTCATAAATGGTAAGTCCAAGAAAAATACAGGTATTCCCCCCCAAAG
AAAACTGTAAAATCGACTTTTTTCTATCTGTACTGTTTTTTATTGGTTTT
TAAATTGGTTTTCCAAGTGAGTAAATCAGAATCTATCTGTAATGGATTTT
AAATTTAGTGTTTCTCTGTGATGTAGTAAACAAGAAACTAGAGGCAAAAA
TAGCCCTGTCCCTTGCTAAACTTCTAAGGCACTTTTCTAGTACAACTCAA
CACTAACATTTCAGGCCTTTAGTGCCTTATATGAGTTTTTAAAAGGGGGA
AAAGGGAGGGAGCAAGAGTGTCTTAACTCATACATTTAGGCATAACAATT
ATTCTCATATTTTAGTTATTGAGAGGGCTGGTAGAAAAACTAGGTAAATA
ATATTAATAATTATAGCGCTTATTAAACACTACAGAACACTTACTATGTA
CCAGGCATTGTGGGAGGCTCTCTCTTGTGCATTATCTCATTTCATTAGGT
CCATGGAGAGTATTGCATTTTCTTAGTTTAGGCATGGCCTCCACAATAAA
GATTATCAAAAGCCTAAAAATATGTAAAAGAAACCTAGAAGTTATTTGTT
GTGCTCCTTGGGGAAGCTAGGCAAATCCTTTCAACTGAAAACCATGGTGA
CTTCCAAGATCTCTGCCCCTCCCCATCGCCATGGTCCACTTCCTCTTCTC
ACTGTTCCTCTTAGAAAAGATCTGTGGACTCCACCACCACGAAATGGCGG
CACCTTATTTATGGTCACTTTAGAGGGTAGGTTTTCTTAATGGGTCTGCC
TGTCATGTTTAACGTCCTTGGCTGGGTCCAAGGCAGATGCAGTCCAAACT
CTCACTAAAATTGCCGAGCCCTTTGTCTTCCAGTGTCTAAAATATTAATG
TCAATGGAATCAGGCCAGAGTTTGAATTCTAGTCTCTTAGCCTTTGTTTC
CCCTGTCCATAAAATGAATGGGGGTAATTCTTTCCTCCTACAGTTTATTT
ATATATTCACTAATTCATTCATTCATCCATCCATTCGTTCATTCGGTTTA
CTGAGTACCTACTATGTGCCAGCCCCTGTTCTAGGGTGGAAACTAAGAGA
ATGATGTACCTAGAGGGCGCTGGAAGCTCTAAAGCCCTAGCAGTTACTGC
TTTTACTATTAGTGGTCGTTTTTTTCTCCCCCCCGCCCCCCGACAAATCA
ACAGAACAAAGAAAATTACCTAAACAGCAAGGACATAGGGAGGAACTTCT
TGGCACAGAACTTTCCAAACACTTTTTCCTGAAGGGATACAAGAAGCAAG
AAAGGTACTCTTTCACTAGGACCTTCTCTGAGCTGTCCTCAGGATGCTTT
TGGGACTATTTTTCTTACCCAGAGAATGGAGAAACCCTGCAGGGAATTCC
CAAGCTGTAGTTATAAACAGAAGTTCTCCTTCTGCTAGGTAGCATTCAAA
GATCTTAATCTTCTGGGTTTCCGTTTTCTCGAATGAAAAATGCAGGTCCG
AGCAGTTAACTGGCTGGGGCACCATTAGCAAGTCACTTAGCATCTCTGGG
GCCAGTCTGCAAAGCGAGGGGGCAGCCTTAATGTGCCTCCAGCCTGAAGT
CCTAGAATGAGCGCCCGGTGTCCCAAGCTGGGGCGCGCACCCCAGATCGG
AGGGCGCCGATGTACAGACAGCAAACTCACCCAGTCTAGTGCATGCCTTC
TTAAACATCACGAGACTCTAAGAAAAGGAAACTGAAAACGGGAAAGTCCC
TCTCTCTAACCTGGCACTGCGTCGCTGGCTTGGAGACAGGTGACGGTCCC
TGCGGGCCTTGTCCTGATTGGCTGGGCACGCGTTTAATATAAGTGGAGGC
GTCGCGCTGGCGGGCATTCCTGAAGCTGACAGCATTCGGGCCGAGATGTC
TCGCTCCGTGGCCTTAGCTGTGCTCGCGCTACTCTCTCTTTCTGGCCTGG
AGGCTATCCAGCGTGAGTCTCTCCTACCCTCCCGCTCTGGTCCTTCCTCT
CCCGCTCTGCACCCTCTGTGGCCCTCGCTGTGCTCTCTCGCTCCGTGACT
TCCCTTCTCCAAGTTCTCCTTGGTGGCCCGCCGTGGGGCTAGTCCAGGGC
TGGATCTCGGGGAAGCGGCGGGGTGGCCTGGGAGTGGGGAAGGGGGTGCG
CACCCGGGACGCGCGCTACTTGCCCCTTTCGGCGGGGAGCAGGGGAGACC
TTTGGCCTACGGCGACGGGAGGGTCGGGACAAAGTTTAGGGCGTCGATAA
GCGTCAGAGCGCCGAGGTTGGGGGAGGGTTTCTCTTCCGCTCTTTCGCGG
GGCCTCTGGCTCCCCCAGCGCAGCTGGAGTGGGGGACGGGTAGGCTCGTC
CCAAAGGCGCGGCGCTGAGGTTTGTGAACGCGTGGAGGGGCGCTTGGGGT
CTGGGGGAGGCGTCGCCCGGGTAAGCCTGTCTGCTGCGGCTCTGCTTCCC
TTAGACTGGAGAGCTGTGGACTTCGTCTAGGCGCCCGCTAAGTTCGCATG
TCCTAGCACCTCTGGGTCTATGTGGGGCCACACCGTGGGGAGGAAACAGC
ACGCGACGTTTGTAGAATGCTTGGCTGTGATACAAAGCGGTTTCGAATAA
TTAACTTATTTGTTCCCATCACATGTCACTTTTAAAAAATTATAAGAACT
ACCCGTTATTGACATCTTTCTGTGTGCCAAGGACTTTATGTGCTTTGCGT
CATTTAATTTTGAAAACAGTTATCTTCCGCCATAGATAACTACTATGGTT
ATCTTCTGCCTCTCACAGATGAAGAAACTAAGGCACCGAGATTTTAAGAA
ACTTAATTACACAGGGGATAAATGGCAGCAATCGAGATTGAAGTCAAGCC
TAACCAGGGCTTTTGCGGGAGCGCATGCCTTTTGGCTGTAATTCGTGCAT
TTTTTTTTAAGAAAAACGCCTGCCTTCTGCGTGAGATTCTCCAGAGCAAA
CTGGGCGGCATGGGCCCTGTGGTCTTTTCGTACAGAGGGCTTCCTCTTTG
GCTCTTTGCCTGGTTGTTTCCAAGATGTACTGTGCCTCTTACTTTCGGTT
TTGAAAACATGAGGGGGTTGGGCGTGGTAGCTTACGCCTGTAATCCCAGC
ACTTAGGGAGGCCGAGGCGGGAGGATGGCTTGAGGTCCGTAGTTGAGACC
AGCCTGGCCAACATGGTGAAGCCTGGTCTCTACAAAAAATAATAACAAAA
ATTAGCCGGGTGTGGTGGCTCGTGCCTGTGGTCCCAGCTGCTCCGGTGGC
TGAGGCGGGAGGATCTCTTGAGCTTAGGCTTTTGAGCTATCATGGCGCCA
GTGCACTCCAGCGTGGGCAACAGAGCGAGACCCTGTCTCTCAAAAAAGAA
AAAAAAAAAAAAAGAAAGAGAAAAGAAAAGAAAGAAAGAAGTGAAGGTTT
GTCAGTCAGGGGAGCTGTAAAACCATTAATAAAGATAATCCAAGATGGTT
ACCAAGACTGTTGAGGACGCCAGAGATCTTGAGCACTTTCTAAGTACCTG
GCAATACACTAAGCGCGCTCACCTTTTCCTCTGGCAAAACATGATCGAAA
GCAGAATGTTTTGATCATGAGAAAATTGCATTTAATTTGAATACAATTTA
TTTACAACATAAAGGATAATGTATATATCACCACCATTACTGGTATTTGC
TGGTTATGTTAGATGTCATTTTAAAAAATAACAATCTGATATTTAAAAAA
AAATCTTATTTTGAAAATTTCCAAAGTAATACATGCCATGCATAGACCAT
TTCTGGAAGATACCACAAGAAACATGTAATGATGATTGCCTCTGAAGGTC
TATTTTCCTCCTCTGACCTGTGTGTGGGTTTTGTTTTTGTTTTACTGTGG
GCATAAATTAATTTTTCAGTTAAGTTTTGGAAGCTTAAATAACTCTCCAA
AAGTCATAAAGCCAGTAACTGGTTGAGCCCAAATTCAAACCCAGCCTGTC
TGATACTTGTCCTCTTCTTAGAAAAGATTACAGTGATGCTCTCACAAAAT
CTTGCCGCCTTCCCTCAAACAGAGAGTTCCAGGCAGGATGAATCTGTGCT
CTGATCCCTGAGGCATTTAATATGTTCTTATTATTAGAAGCTCAGATGCA
AAGAGCTCTCTTAGCTTTTAATGTTATGAAAAAAATCAGGTCTTCATTAG
ATTCCCCAATCCACCTCTTGATGGGGCTAGTAGCCTTTCCTTAATGATAG
GGTGTTTCTAGAGAGATATATCTGGTCAAGGTGGCCTGGTACTCCTCCTT
CTCCCCACAGCCTCCCAGACAAGGAGGAGTAGCTGCCTTTTAGTGATCAT
GTACCCTGAATATAAGTGTATTTAAAAGAATTTTATACACATATATTTAG
TGTCAATCTGTATATTTAGTAGCACTAACACTTCTCTTCATTTTCAATGA
AAAATATAGAGTTTATAATATTTTCTTCCCACTTCCCCATGGATGGTCTA
GTCATGCCTCTCATTTTGGAAAGTACTGTTTCTGAAACATTAGGCAATAT
ATTCCCAACCTGGCTAGTTTACAGCAATCACCTGTGGATGCTAATTAAAA
CGCAAATCCCACTGTCACATGCATTACTCCATTTGATCATAATGGAAAGT
ATGTTCTGTCCCATTTGCCATAGTCCTCACCTATCCCTGTTGTATTTTAT
CGGGTCCAACTCAACCATTTAAGGTATTTGCCAGCTCTTGTATGCATTTA
GGTTTTGTTTCTTTGTTTTTTAGCTCATGAAATTAGGTACAAAGTCAGAG
AGGGGTCTGGCATATAAAACCTCAGCAGAAATAAAGAGGTTTTGTTGTTT
GGTAAGAACATACCTTGGGTTGGTTGGGCACGGTGGCTCGTGCCTGTAAT
CCCAACACTTTGGGAGGCCAAGGCAGGCTGATCACTTGAAGTTGGGAGTT
CAAGACCAGCCTGGCCAACATGGTGAAATCCCGTCTCTACTGAAAATACA
AAAATTAACCAGGCATGGTGGTGTGTGCCTGTAGTCCCAGGAATCACTTG
AACCCAGGAGGCGGAGGTTGCAGTGAGCTGAGATCTCACCACTGCACACT
GCACTCCAGCCTGGGCAATGGAATGAGATTCCATCCCAAAAAATAAAAAA
ATAAAAAAATAAAGAACATACCTTGGGTTGATCCACTTAGGAACCTCAGA
TAATAACATCTGCCACGTATAGAGCAATTGCTATGTCCCAGGCACTCTAC
TAGACACTTCATACAGTTTAGAAAATCAGATGGGTGTAGATCAAGGCAGG
AGCAGGAACCAAAAAGAAAGGCATAAACATAAGAAAAAAAATGGAAGGGG
TGGAAACAGAGTACAATAACATGAGTAATTTGATGGGGGCTATTATGAAC
TGAGAAATGAACTTTGAAAAGTATCTTGGGGCCAAATCATGTAGACTCTT
GAGTGATGTGTTAAGGAATGCTATGAGTGCTGAGAGGGCATCAGAAGTCC
TTGAGAGCCTCCAGAGAAAGGCTCTTAAAAATGCAGCGCAATCTCCAGTG
ACAGAAGATACTGCTAGAAATCTGCTAGAAAAAAAACAAAAAAGGCATGT
ATAGAGGAATTATGAGGGAAAGATACCAAGTCACGGTTTATTCTTCAAAA
TGGAGGTGGCTTGTTGGGAAGGTGGAAGCTCATTTGGCCAGAGTGGAAAT
GGAATTGGGAGAAATCGATGACCAAATGTAAACACTTGGTGCCTGATATA
GCTTGACACCAAGTTAGCCCCAAGTGAAATACCCTGGCAATATTAATGTG
TCTTTTCCCGATATTCCTCAGGTACTCCAAAGATTCAGGTTTACTCACGT
CATCCAGCAGAGAATGGAAAGTCAAATTTCCTGAATTGCTATGTGTCTGG
GTTTCATCCATCCGACATTGAAGTTGACTTACTGAAGAATGGAGAGAGAA
TTGAAAAAGTGGAGCATTCAGACTTGTCTTTCAGCAAGGACTGGTCTTTC
TATCTCTTGTACTACACTGAATTCACCCCCACTGAAAAAGATGAGTATGC
CTGCCGTGTGAACCATGTGACTTTGTCACAGCCCAAGATAGTTAAGTGGG
GTAAGTCTTACATTCTTTTGTAAGCTGCTGAAAGTTGTGTATGAGTAGTC
ATATCATAAAGCTGCTTTGATATAAAAAAGGTCTATGGCCATACTACCCT
GAATGAGTCCCATCCCATCTGATATAAACAATCTGCATATTGGGATTGTC
AGGGAATGTTCTTAAAGATCAGATTAGTGGCACCTGCTGAGATACTGATG
CACAGCATGGTTTCTGAACCAGTAGTTTCCCTGCAGTTGAGCAGGGAGCA
GCAGCAGCACTTGCACAAATACATATACACTCTTAACACTTCTTACCTAC
TGGCTTCCTCTAGCTTTTGTGGCAGCTTCAGGTATATTTAGCACTGAACG
AACATCTCAAGAAGGTATAGGCCTTTGTTTGTAAGTCCTGCTGTCCTAGC
ATCCTATAATCCTGGACTTCTCCAGTACTTTCTGGCTGGATTGGTATCTG
AGGCTAGTAGGAAGGGCTTGTTCCTGCTGGGTAGCTCTAAACAATGTATT
CATGGGTAGGAACAGCAGCCTATTCTGCCAGCCTTATTTCTAACCATTTT
AGACATTTGTTAGTACATGGTATTTTAAAAGTAAAACTTAATGTCTTCCT
TTTTTTTCTCCACTGTCTTTTTCATAGATCGAGACATGTAAGCAGCATCA
TGGAGGTAAGTTTTTGACCTTGAGAAAATGTTTTTGTTTCACTGTCCTGA
GGACTATTTATAGACAGCTCTAACATGATAACCCTCACTATGTGGAGAAC
ATTGACAGAGTAACATTTTAGCAGGGAAAGAAGAATCCTACAGGGTCATG
TTCCCTTCTCCTGTGGAGTGGCATGAAGAAGGTGTATGGCCCCAGGTATG
GCCATATTACTGACCCTCTACAGAGAGGGCAAAGGAACTGCCAGTATGGT
ATTGCAGGATAAAGGCAGGTGGTTACCCACATTACCTGCAAGGCTTTGAT
CTTTCTTCTGCCATTTCCACATTGGACATCTCTGCTGAGGAGAGAAAATG
AACCACTCTTTTCCTTTGTATAATGTTGTTTTATTCTTCAGACAGAAGAG
AGGAGTTATACAGCTCTGCAGACATCCCATTCCTGTATGGGGACTGTGTT
TGCCTCTTAGAGGTTCCCAGGCCACTAGAGGAGATAAAGGGAAACAGATT
GTTATAACTTGATATAATGATACTATAATAGATGTAACTACAAGGAGCTC
CAGAAGCAAGAGAGAGGGAGGAACTTGGACTTCTCTGCATCTTTAGTTGG
AGTCCAAAGGCTTTTCAATGAAATTCTACTGCCCAGGGTACATTGATGCT
GAAACCCCATTCAAATCTCCTGTTATATTCTAGAACAGGGAATTGATTTG
GGAGAGCATCAGGAAGGTGGATGATCTGCCCAGTCACACTGTTAGTAAAT
TGTAGAGCCAGGACCTGAACTCTAATATAGTCATGTGTTACTTAATGACG
GGGACATGTTCTGAGAAATGCTTACACAAACCTAGGTGTTGTAGCCTACT
ACACGCATAGGCTACATGGTATAGCCTATTGCTCCTAGACTACAAACCTG
TACAGCCTGTTACTGTACTGAATACTGTGGGCAGTTGTAACACAATGGTA
AGTATTTGTGTATCTAAACATAGAAGTTGCAGTAAAAATATGCTATTTTA
ATCTTATGAGACCACTGTCATATATACAGTCCATCATTGACCAAAACATC
ATATCAGCATTTTTTCTTCTAAGATTTTGGGAGCACCAAAGGGATACACT
AACAGGATATACTCTTTATAATGGGTTTGGAGAACTGTCTGCAGCTACTT
CTTTTAAAAAGGTGATCTACACAGTAGAAATTAGACAAGTTTGGTAATGA
GATCTGCAATCCAAATAAAATAAATTCATTGCTAACCTTTTTCTTTTCTT
TTCAGGTTTGAAGATGCCGCATTTGGATTGGATGAATTCCAAATTCTGCT
TGCTTGCTTTTTAATATTGATATGCTTATACACTTACACTTTATGCACAA
AATGTAGGGTTATAATAATGTTAACATGGACATGATCTTCTTTATAATTC
TACTTTGAGTGCTGTCTCCATGTTTGATGTATCTGAGCAGGTTGCTCCAC
AGGTAGCTCTAGGAGGGCTGGCAACTTAGAGGTGGGGAGCAGAGAATTCT
CTTATCCAACATCAACATCTTGGTCAGATTTGAACTCTTCAATCTCTTGC
ACTCAAAGCTTGTTAAGATAGTTAAGCGTGCATAAGTTAACTTCCAATTT
ACATACTCTGCTTAGAATTTGGGGGAAAATTTAGAAATATAATTGACAGG
ATTATTGGAAATTTGTTATAATGAATGAAACATTTTGTCATATAAGATTC
ATATTTACTTCTTATACATTTGATAAAGTAAGGCATGGTTGTGGTTAATC
TGGTTTATTTTTGTTCCACAAGTTAAATAAATCATAAAACTTGATGTGTT
ATCTCTTATATCTCACTCCCACTATTACCCCTTTATTTTCAAACAGGGAA
ACAGTCTTCAAGTTCCACTTGGTAAAAAATGTGAACCCCTTGTATATAGA
GTTTGGCTCACAGTGTAAAGGGCCTCAGTGATTCACATTTTCCAGATTAG
GAATCTGATGCTCAAAGAAGTTAAATGGCATAGTTGGGGTGACACAGCTG
TCTAGTGGGAGGCCAGCCTTCTATATTTTAGCCAGCGTTCTTTCCTGCGG
GCCAGGTCATGAGGAGTATGCAGACTCTAAGAGGGAGCAAAAGTATCTGA
AGGATTTAATATTTTAGCAAGGAATAGATATACAATCATCCCTTGGTCTC
CCTGGGGGATTGGTTTCAGGACCCCTTCTTGGACACCAAATCTATGGATA
TTTAAGTCCCTTCTATAAAATGGTATAGTATTTGCATATAACCTATCCAC
ATCCTCCTGTATACTTTAAATCATTTCTAGATTACTTGTAATACCTAATA
CAATGTAAATGCTATGCAAATAGTTGTTATTGTTTAAGGAATAATGACAA
GAAAAAAAAGTCTGTACATGCTCAGTAAAGACACAACCATCCCTTTTTTT
CCCCAGTGTTTTTGATCCATGGTTTGCTGAATCCACAGATGTGGAGCCCC
TGGATACGGAAGGCCCGCTGTACTTTGAATGACAAATAACAGATTTAAA

The term “Cas9” or “Cas9 domain” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (“clustered regularly interspaced short palindromic repeat”)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase.

A nuclease-inactivated Cas9 protein may interchangeably be referred to as a “dCas9” protein (for nuclease-“dead” Cas9). Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, the fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length. In some embodiments, wild type Cas9 corresponds to Cas9 from Streptococcus pyogenes (NCBI Reference Sequence: NC_017053.1, nucleotide and amino acid sequences as follows).

ATGGATAAGAAATACTCAATAGGCTTAGATATCGGCACAAATAGCGTCGGATGGGC
GGTGATCACTGATGATTATAAGGTTCCGTCTAAAAAGTTCAAGGTTCTGGGAAATAC
AGACCGCCACAGTATCAAAAAAAATCTTATAGGGGCTCTTTTATTTGGCAGTGGAGA
GACAGCGGAAGCGACTCGTCTCAAACGGACAGCTCGTAGAAGGTATACACGTCGGA
AGAATCGTATTTGTTATCTACAGGAGATTTTTTCAAATGAGATGGCGAAAGTAGATG
ATAGTTTCTTTCATCGACTTGAAGAGTCTTTTTTGGTGGAAGAAGACAAGAAGCATG
AACGTCATCCTATTTTTGGAAATATAGTAGATGAAGTTGCTTATCATGAGAAATATC
CAACTATCTATCATCTGCGAAAAAAATTGGCAGATTCTACTGATAAAGCGGATTTGC
GCTTAATCTATTTGGCCTTAGCGCATATGATTAAGTTTCGTGGTCATTTTTTGATTGA
GGGAGATTTAAATCCTGATAATAGTGATGTGGACAAACTATTTATCCAGTTGGTACA
AATCTACAATCAATTATTTGAAGAAAACCCTATTAACGCAAGTAGAGTAGATGCTAA
AGCGATTCTTTCTGCACGATTGAGTAAATCAAGACGATTAGAAAATCTCATTGCTCA
GCTCCCCGGTGAGAAGAGAAATGGCTTGTTTGGGAATCTCATTGCTTTGTCATTGGG
ATTGACCCCTAATTTTAAATCAAATTTTGATTTGGCAGAAGATGCTAAATTACAGCT
TTCAAAAGATACTTACGATGATGATTTAGATAATTTATTGGCGCAAATTGGAGATCA
ATATGCTGATTTGTTTTTGGCAGCTAAGAATTTATCAGATGCTATTTTACTTTCAGAT
ATCCTAAGAGTAAATAGTGAAATAACTAAGGCTCCCCTATCAGCTTCAATGATTAAG
CGCTACGATGAACATCATCAAGACTTGACTCTTTTAAAAGCTTTAGTTCGACAACAA
CTTCCAGAAAAGTATAAAGAAATCTTTTTTGATCAATCAAAAAACGGATATGCAGGT
TATATTGATGGGGGAGCTAGCCAAGAAGAATTTTATAAATTTATCAAACCAATTTTA
GAAAAAATGGATGGTACTGAGGAATTATTGGTGAAACTAAATCGTGAAGATTTGCT
GCGCAAGCAACGGACCTTTGACAACGGCTCTATTCCCCATCAAATTCACTTGGGTGA
GCTGCATGCTATTTTGAGAAGACAAGAAGACTTTTATCCATTTTTAAAAGACAATCG
TGAGAAGATTGAAAAAATCTTGACTTTTCGAATTCCTTATTATGTTGGTCCATTGGCG
CGTGGCAATAGTCGTTTTGCATGGATGACTCGGAAGTCTGAAGAAACAATTACCCCA
TGGAATTTTGAAGAAGTTGTCGATAAAGGTGCTTCAGCTCAATCATTTATTGAACGC
ATGACAAACTTTGATAAAAATCTTCCAAATGAAAAAGTACTACCAAAACATAGTTTG
CTTTATGAGTATTTTACGGTTTATAACGAATTGACAAAGGTCAAATATGTTACTGAG
GGAATGCGAAAACCAGCATTTCTTTCAGGTGAACAGAAGAAAGCCATTGTTGATTTA
CTCTTCAAAACAAATCGAAAAGTAACCGTTAAGCAATTAAAAGAAGATTATTTCAA
AAAAATAGAATGTTTTGATAGTGTTGAAATTTCAGGAGTTGAAGATAGATTTAATGC
TTCATTAGGCGCCTACCATGATTTGCTAAAAATTATTAAAGATAAAGATTTTTTGGA
TAATGAAGAAAATGAAGATATCTTAGAGGATATTGTTTTAACATTGACCTTATTTGA
AGATAGGGGGATGATTGAGGAAAGACTTAAAACATATGCTCACCTCTTTGATGATA
AGGTGATGAAACAGCTTAAACGTCGCCGTTATACTGGTTGGGGACGTTTGTCTCGAA
AATTGATTAATGGTATTAGGGATAAGCAATCTGGCAAAACAATATTAGATTTTTTGA
AATCAGATGGTTTTGCCAATCGCAATTTTATGCAGCTGATCCATGATGATAGTTTGA
CATTTAAAGAAGATATTCAAAAAGCACAGGTGTCTGGACAAGGCCATAGTTTACAT
GAACAGATTGCTAACTTAGCTGGCAGTCCTGCTATTAAAAAAGGTATTTTACAGACT
GTAAAAATTGTTGATGAACTGGTCAAAGTAATGGGGCATAAGCCAGAAAATATCGT
TATTGAAATGGCACGTGAAAATCAGACAACTCAAAAGGGCCAGAAAAATTCGCGAG
AGCGTATGAAACGAATCGAAGAAGGTATCAAAGAATTAGGAAGTCAGATTCTTAAA
GAGCATCCTGTTGAAAATACTCAATTGCAAAATGAAAAGCTCTATCTCTATTATCTA
CAAAATGGAAGAGACATGTATGTGGACCAAGAATTAGATATTAATCGTTTAAGTGA
TTATGATGTCGATCACATTGTTCCACAAAGTTTCATTAAAGACGATTCAATAGACAA
TAAGGTACTAACGCGTTCTGATAAAAATCGTGGTAAATCGGATAACGTTCCAAGTGA
AGAAGTAGTCAAAAAGATGAAAAACTATTGGAGACAACTTCTAAACGCCAAGTTAA
TCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCCAAATCACTAAGC
ATGTGGCACAAATTTTGGATAGTCGCATGAATACTAAATACGATGAAAATGATAAA
CTTATTCGAGAGGTTAAAGTGATTACCTTAAAATCTAAATTAGTTTCTGACTTCCGA
AAAGATTTCCAATTCTATAAAGTACGTGAGATTAACAATTACCATCATGCCCATGAT
GCGTATCTAAATGCCGTCGTTGGAACTGCTTTGATTAAGAAATATCCAAAACTTGAA
TCGGAGTTTGTCTATGGTGATTATAAAGTTTATGATGTTCGTAAAATGATTGCTAAGT
CTGAGCAAGAAATAGGCAAAGCAACCGCAAAATATTTCTTTTACTCTAATATCATGA
ACTTCTTCAAAACAGAAATTACACTTGCAAATGGAGAGATTCGCAAACGCCCTCTAA
TCGAAACTAATGGGGAAACTGGAGAAATTGTCTGGGATAAAGGGCGAGATTTTGCC
ACAGTGCGCAAAGTATTGTCCATGCCCCAAGTCAATATTGTCAAGAAAACAGAAGT
ACAGACAGGCGGATTCTCCAAGGAGTCAATTTTACCAAAAAGAAATTCGGACAAGC
TTATTGCTCGTAAAAAAGACTGGGATCCAAAAAAATATGGTGGTTTTGATAGTCCAA
CGGTAGCTTATTCAGTCCTAGTGGTTGCTAAGGTGGAAAAAGGGAAATCGAAGAAG
TTAAAATCCGTTAAAGAGTTACTAGGGATCACAATTATGGAAAGAAGTTCCTTTGAA
AAAAATCCGATTGACTTTTTAGAAGCTAAAGGATATAAGGAAGTTAAAAAAGACTT
AATCATTAAACTACCTAAATATAGTCTTTTTGAGTTAGAAAACGGTCGTAAACGGAT
GCTGGCTAGTGCCGGAGAATTACAAAAAGGAAATGAGCTGGCTCTGCCAAGCAAAT
ATGTGAATTTTTTATATTTAGCTAGTCATTATGAAAAGTTGAAGGGTAGTCCAGAAG
ATAACGAACAAAAACAATTGTTTGTGGAGCAGCATAAGCATTATTTAGATGAGATT
ATTGAGCAAATCAGTGAATTTTCTAAGCGTGTTATTTTAGCAGATGCCAATTTAGAT
AAAGTTCTTAGTGCATATAACAAACATAGAGACAAACCAATACGTGAACAAGCAGA
AAATATTATTCATTTATTTACGTTGACGAATCTTGGAGCTCCCGCTGCTTTTAAATAT
TTTGATACAACAATTGATCGTAAACGATATACGTCTACAAAAGAAGTTTTAGATGCC
ACTCTTATCCATCAATCCATCACTGGTCTTTATGAAACACGCATTGATTTGAGTCAGC
TAGGAGGTGACTGA
MDKKYSIGLDIGTNSVGWAVITDDYKVPSKKFKVLGNTDRHSIKKNLIGALLFGSGETA
EATRLKRTARRRYTRRKNRICYLQEIFSNEMAKVDDSFFHRLEESFLVEEDKKHERHPIF
GNIVDEVAYHEKYPTIYHLRKKLADSTDKADLRLIYLALAHMIKFRGHFLIEGDLNPDNS
DVDKLFIQLVQIYNQLFEENPINASRVDAKAILSARLSKSRRLENLIAQLPGEKRNGLFGN
LIALSLGLTPNFKSNFDLAEDAKLQLSKDTYDDDLDNLLAQIGDQYADLFLAAKNLSDA
ILLSDILRVNSEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNASLGAYHDLL
KIIKDKDFLDNEENEDILEDIVLTLTLFEDRGMIEERLKTYAHLFDDKVMKQLKRRRYTG
WGRLSRKLINGIRDKQSGKTILDFLKSDGFANRNFMQLIHDDSLTFKEDIQKAQVSGQG
HSLHEQIANLAGSPAIKKGILQTVKIVDELVKVMGHKPENIVIEMARENQTTQKGQKNS
RERMKRIEEGIKELGSQILKEHPVENTQLQNEKLYLYYLQNGRDMYVDQELDINRLSDY
DVDHIVPQSFIKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLIETNGETGEIV
WDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGD
(single underline: HNH domain; double underline: RuvC domain)

In some embodiments, wild type Cas9 corresponds to, or comprises the following nucleotide and/or amino acid sequences:

ATGGATAAAAAGTATTCTATTGGTTTAGACATCGGCACTAATTCCGTTGGATGGGCT
GTCATAACCGATGAATACAAAGTACCTTCAAAGAAATTTAAGGTGTTGGGGAACAC
AGACCGTCATTCGATTAAAAAGAATCTTATCGGTGCCCTCCTATTCGATAGTGGCGA
AACGGCAGAGGCGACTCGCCTGAAACGAACCGCTCGGAGAAGGTATACACGTCGCA
AGAACCGAATATGTTACTTACAAGAAATTTTTAGCAATGAGATGGCCAAAGTTGAC
GATTCTTTCTTTCACCGTTTGGAAGAGTCCTTCCTTGTCGAAGAGGACAAGAAACAT
GAACGGCACCCCATCTTTGGAAACATAGTAGATGAGGTGGCATATCATGAAAAGTA
CCCAACGATTTATCACCTCAGAAAAAAGCTAGTTGACTCAACTGATAAAGCGGACCT
GAGGTTAATCTACTTGGCTCTTGCCCATATGATAAAGTTCCGTGGGCACTTTCTCATT
GAGGGTGATCTAAATCCGGACAACTCGGATGTCGACAAACTGTTCATCCAGTTAGTA
CAAACCTATAATCAGTTGTTTGAAGAGAACCCTATAAATGCAAGTGGCGTGGATGC
GAAGGCTATTCTTAGCGCCCGCCTCTCTAAATCCCGACGGCTAGAAAACCTGATCGC
ACAATTACCCGGAGAGAAGAAAAATGGGTTGTTCGGTAACCTTATAGCGCTCTCACT
AGGCCTGACACCAAATTTTAAGTCGAACTTCGACTTAGCTGAAGATGCCAAATTGCA
GCTTAGTAAGGACACGTACGATGACGATCTCGACAATCTACTGGCACAAATTGGAG
ATCAGTATGCGGACTTATTTTTGGCTGCCAAAAACCTTAGCGATGCAATCCTCCTAT
CTGACATACTGAGAGTTAATACTGAGATTACCAAGGCGCCGTTATCCGCTTCAATGA
TCAAAAGGTACGATGAACATCACCAAGACTTGACACTTCTCAAGGCCCTAGTCCGTC
AGCAACTGCCTGAGAAATATAAGGAAATATTCTTTGATCAGTCGAAAAACGGGTAC
GCAGGTTATATTGACGGCGGAGCGAGTCAAGAGGAATTCTACAAGTTTATCAAACC
CATATTAGAGAAGATGGATGGGACGGAAGAGTTGCTTGTAAAACTCAATCGCGAAG
ATCTACTGCGAAAGCAGCGGACTTTCGACAACGGTAGCATTCCACATCAAATCCACT
TAGGCGAATTGCATGCTATACTTAGAAGGCAGGAGGATTTTTATCCGTTCCTCAAAG
ACAATCGTGAAAAGATTGAGAAAATCCTAACCTTTCGCATACCTTACTATGTGGGAC
CCCTGGCCCGAGGGAACTCTCGGTTCGCATGGATGACAAGAAAGTCCGAAGAAACG
ATTACTCCATGGAATTTTGAGGAAGTTGTCGATAAAGGTGCGTCAGCTCAATCGTTC
ATCGAGAGGATGACCAACTTTGACAAGAATTTACCGAACGAAAAAGTATTGCCTAA
GCACAGTTTACTTTACGAGTATTTCACAGTGTACAATGAACTCACGAAAGTTAAGTA
TGTCACTGAGGGCATGCGTAAACCCGCCTTTCTAAGCGGAGAACAGAAGAAAGCAA
TAGTAGATCTGTTATTCAAGACCAACCGCAAAGTGACAGTTAAGCAATTGAAAGAG
GACTACTTTAAGAAAATTGAATGCTTCGATTCTGTCGAGATCTCCGGGGTAGAAGAT
CGATTTAATGCGTCACTTGGTACGTATCATGACCTCCTAAAGATAATTAAAGATAAG
GACTTCCTGGATAACGAAGAGAATGAAGATATCTTAGAAGATATAGTGTTGACTCTT
ACCCTCTTTGAAGATCGGGAAATGATTGAGGAAAGACTAAAAACATACGCTCACCT
GTTCGACGATAAGGTTATGAAACAGTTAAAGAGGCGTCGCTATACGGGCTGGGGAC
GATTGTCGCGGAAACTTATCAACGGGATAAGAGACAAGCAAAGTGGTAAAACTATT
CTCGATTTTCTAAAGAGCGACGGCTTCGCCAATAGGAACTTTATGCAGCTGATCCAT
GATGACTCTTTAACCTTCAAAGAGGATATACAAAAGGCACAGGTTTCCGGACAAGG
GGACTCATTGCACGAACATATTGCGAATCTTGCTGGTTCGCCAGCCATCAAAAAGGG
CATACTCCAGACAGTCAAAGTAGTGGATGAGCTAGTTAAGGTCATGGGACGTCACA
AACCGGAAAACATTGTAATCGAGATGGCACGCGAAAATCAAACGACTCAGAAGGG
GCAAAAAAACAGTCGAGAGCGGATGAAGAGAATAGAAGAGGGTATTAAAGAACTG
GGCAGCCAGATCTTAAAGGAGCATCCTGTGGAAAATACCCAATTGCAGAACGAGAA
ACTTTACCTCTATTACCTACAAAATGGAAGGGACATGTATGTTGATCAGGAACTGGA
CATAAACCGTTTATCTGATTACGACGTCGATCACATTGTACCCCAATCCTTTTTGAAG
GACGATTCAATCGACAATAAAGTGCTTACACGCTCGGATAAGAACCGAGGGAAAAG
TGACAATGTTCCAAGCGAGGAAGTCGTAAAGAAAATGAAGAACTATTGGCGGCAGC
TCCTAAATGCGAAACTGATAACGCAAAGAAAGTTCGATAACTTAACTAAAGCTGAG
AGGGGTGGCTTGTCTGAACTTGACAAGGCCGGATTTATTAAACGTCAGCTCGTGGAA
ACCCGCCAAATCACAAAGCATGTTGCACAGATACTAGATTCCCGAATGAATACGAA
ATACGACGAGAACGATAAGCTGATTCGGGAAGTCAAAGTAATCACTTTAAAGTCAA
AATTGGTGTCGGACTTCAGAAAGGATTTTCAATTCTATAAAGTTAGGGAGATAAATA
ACTACCACCATGCGCACGACGCTTATCTTAATGCCGTCGTAGGGACCGCACTCATTA
AGAAATACCCGAAGCTAGAAAGTGAGTTTGTGTATGGTGATTACAAAGTTTATGAC
GTCCGTAAGATGATCGCGAAAAGCGAACAGGAGATAGGCAAGGCTACAGCCAAAT
ACTTCTTTTATTCTAACATTATGAATTTCTTTAAGACGGAAATCACTCTGGCAAACGG
AGAGATACGCAAACGACCTTTAATTGAAACCAATGGGGAGACAGGTGAAATCGTAT
GGGATAAGGGCCGGGACTTCGCGACGGTGAGAAAAGTTTTGTCCATGCCCCAAGTC
AACATAGTAAAGAAAACTGAGGTGCAGACCGGAGGGTTTTCAAAGGAATCGATTCT
TCCAAAAAGGAATAGTGATAAGCTCATCGCTCGTAAAAAGGACTGGGACCCGAAAA
AGTACGGTGGCTTCGATAGCCCTACAGTTGCCTATTCTGTCCTAGTAGTGGCAAAAG
TTGAGAAGGGAAAATCCAAGAAACTGAAGTCAGTCAAAGAATTATTGGGGATAACG
ATTATGGAGCGCTCGTCTTTTGAAAAGAACCCCATCGACTTCCTTGAGGCGAAAGGT
TACAAGGAAGTAAAAAAGGATCTCATAATTAAACTACCAAAGTATAGTCTGTTTGA
GTTAGAAAATGGCCGAAAACGGATGTTGGCTAGCGCCGGAGAGCTTCAAAAGGGGA
ACGAACTCGCACTACCGTCTAAATACGTGAATTTCCTGTATTTAGCGTCCCATTACG
AGAAGTTGAAAGGTTCACCTGAAGATAACGAACAGAAGCAACTTTTTGTTGAGCAG
CACAAACATTATCTCGACGAAATCATAGAGCAAATTTCGGAATTCAGTAAGAGAGT
CATCCTAGCTGATGCCAATCTGGACAAAGTATTAAGCGCATACAACAAGCACAGGG
ATAAACCCATACGTGAGCAGGCGGAAAATATTATCCATTTGTTTACTCTTACCAACC
TCGGCGCTCCAGCCGCATTCAAGTATTTTGACACAACGATAGATCGCAAACGATACA
CTTCTACCAAGGAGGTGCTAGACGCGACACTGATTCACCAATCCATCACGGGATTAT
ATGAAACTCGGATAGATTTGTCACAGCTTGGGGGTGACGGATCCCCCAAGAAGAAG
AGGAAAGTCTCGAGCGACTACAAAGACCATGACGGTGATTATAAAGATCATGACAT
CGATTACAAGGATGACGATGACAAGGCTGCAGGA
MDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
(single underline: HNH domain; double underline: RuvC domain)

In some embodiments, wild type Cas9 corresponds to Cas9 from Streptococcus pyogenes (NCBI Reference Sequence: NC_002737.2 (nucleotide sequence as follows); and Uniprot Reference Sequence: Q99ZW2 (amino acid sequence as follows).

ATGGATAAGAAATACTCAATAGGCTTAGATATCGGCACAAATAGCGTCGGATGGGC
GGTGATCACTGATGAATATAAGGTTCCGTCTAAAAAGTTCAAGGTTCTGGGAAATAC
AGACCGCCACAGTATCAAAAAAAATCTTATAGGGGCTCTTTTATTTGACAGTGGAGA
GACAGCGGAAGCGACTCGTCTCAAACGGACAGCTCGTAGAAGGTATACACGTCGGA
AGAATCGTATTTGTTATCTACAGGAGATTTTTTCAAATGAGATGGCGAAAGTAGATG
ATAGTTTCTTTCATCGACTTGAAGAGTCTTTTTTGGTGGAAGAAGACAAGAAGCATG
AACGTCATCCTATTTTTGGAAATATAGTAGATGAAGTTGCTTATCATGAGAAATATC
CAACTATCTATCATCTGCGAAAAAAATTGGTAGATTCTACTGATAAAGCGGATTTGC
GCTTAATCTATTTGGCCTTAGCGCATATGATTAAGTTTCGTGGTCATTTTTTGATTGA
GGGAGATTTAAATCCTGATAATAGTGATGTGGACAAACTATTTATCCAGTTGGTACA
AACCTACAATCAATTATTTGAAGAAAACCCTATTAACGCAAGTGGAGTAGATGCTA
AAGCGATTCTTTCTGCACGATTGAGTAAATCAAGACGATTAGAAAATCTCATTGCTC
AGCTCCCCGGTGAGAAGAAAAATGGCTTATTTGGGAATCTCATTGCTTTGTCATTGG
GTTTGACCCCTAATTTTAAATCAAATTTTGATTTGGCAGAAGATGCTAAATTACAGC
TTTCAAAAGATACTTACGATGATGATTTAGATAATTTATTGGCGCAAATTGGAGATC
AATATGCTGATTTGTTTTTGGCAGCTAAGAATTTATCAGATGCTATTTTACTTTCAGA
TATCCTAAGAGTAAATACTGAAATAACTAAGGCTCCCCTATCAGCTTCAATGATTAA
ACGCTACGATGAACATCATCAAGACTTGACTCTTTTAAAAGCTTTAGTTCGACAACA
ACTTCCAGAAAAGTATAAAGAAATCTTTTTTGATCAATCAAAAAACGGATATGCAG
GTTATATTGATGGGGGAGCTAGCCAAGAAGAATTTTATAAATTTATCAAACCAATTT
TAGAAAAAATGGATGGTACTGAGGAATTATTGGTGAAACTAAATCGTGAAGATTTG
CTGCGCAAGCAACGGACCTTTGACAACGGCTCTATTCCCCATCAAATTCACTTGGGT
GAGCTGCATGCTATTTTGAGAAGACAAGAAGACTTTTATCCATTTTTAAAAGACAAT
CGTGAGAAGATTGAAAAAATCTTGACTTTTCGAATTCCTTATTATGTTGGTCCATTGG
CGCGTGGCAATAGTCGTTTTGCATGGATGACTCGGAAGTCTGAAGAAACAATTACCC
CATGGAATTTTGAAGAAGTTGTCGATAAAGGTGCTTCAGCTCAATCATTTATTGAAC
GCATGACAAACTTTGATAAAAATCTTCCAAATGAAAAAGTACTACCAAAACATAGT
TTGCTTTATGAGTATTTTACGGTTTATAACGAATTGACAAAGGTCAAATATGTTACTG
AAGGAATGCGAAAACCAGCATTTCTTTCAGGTGAACAGAAGAAAGCCATTGTTGAT
TTACTCTTCAAAACAAATCGAAAAGTAACCGTTAAGCAATTAAAAGAAGATTATTTC
AAAAAAATAGAATGTTTTGATAGTGTTGAAATTTCAGGAGTTGAAGATAGATTTAAT
GCTTCATTAGGTACCTACCATGATTTGCTAAAAATTATTAAAGATAAAGATTTTTTG
GATAATGAAGAAAATGAAGATATCTTAGAGGATATTGTTTTAACATTGACCTTATTT
GAAGATAGGGAGATGATTGAGGAAAGACTTAAAACATATGCTCACCTCTTTGATGA
TAAGGTGATGAAACAGCTTAAACGTCGCCGTTATACTGGTTGGGGACGTTTGTCTCG
AAAATTGATTAATGGTATTAGGGATAAGCAATCTGGCAAAACAATATTAGATTTTTT
GAAATCAGATGGTTTTGCCAATCGCAATTTTATGCAGCTGATCCATGATGATAGTTT
GACATTTAAAGAAGACATTCAAAAAGCACAAGTGTCTGGACAAGGCGATAGTTTAC
ATGAACATATTGCAAATTTAGCTGGTAGCCCTGCTATTAAAAAAGGTATTTTACAGA
CTGTAAAAGTTGTTGATGAATTGGTCAAAGTAATGGGGCGGCATAAGCCAGAAAAT
ATCGTTATTGAAATGGCACGTGAAAATCAGACAACTCAAAAGGGCCAGAAAAATTC
GCGAGAGCGTATGAAACGAATCGAAGAAGGTATCAAAGAATTAGGAAGTCAGATTC
TTAAAGAGCATCCTGTTGAAAATACTCAATTGCAAAATGAAAAGCTCTATCTCTATT
ATCTCCAAAATGGAAGAGACATGTATGTGGACCAAGAATTAGATATTAATCGTTTAA
GTGATTATGATGTCGATCACATTGTTCCACAAAGTTTCCTTAAAGACGATTCAATAG
ACAATAAGGTCTTAACGCGTTCTGATAAAAATCGTGGTAAATCGGATAACGTTCCAA
GTGAAGAAGTAGTCAAAAAGATGAAAAACTATTGGAGACAACTTCTAAACGCCAAG
TTAATCACTCAACGTAAGTTTGATAATTTAACGAAAGCTGAACGTGGAGGTTTGAGT
GAACTTGATAAAGCTGGTTTTATCAAACGCCAATTGGTTGAAACTCGCCAAATCACT
AAGCATGTGGCACAAATTTTGGATAGTCGCATGAATACTAAATACGATGAAAATGA
TAAACTTATTCGAGAGGTTAAAGTGATTACCTTAAAATCTAAATTAGTTTCTGACTTC
CGAAAAGATTTCCAATTCTATAAAGTACGTGAGATTAACAATTACCATCATGCCCAT
GATGCGTATCTAAATGCCGTCGTTGGAACTGCTTTGATTAAGAAATATCCAAAACTT
GAATCGGAGTTTGTCTATGGTGATTATAAAGTTTATGATGTTCGTAAAATGATTGCT
AAGTCTGAGCAAGAAATAGGCAAAGCAACCGCAAAATATTTCTTTTACTCTAATATC
ATGAACTTCTTCAAAACAGAAATTACACTTGCAAATGGAGAGATTCGCAAACGCCCT
CTAATCGAAACTAATGGGGAAACTGGAGAAATTGTCTGGGATAAAGGGCGAGATTT
TGCCACAGTGCGCAAAGTATTGTCCATGCCCCAAGTCAATATTGTCAAGAAAACAG
AAGTACAGACAGGCGGATTCTCCAAGGAGTCAATTTTACCAAAAAGAAATTCGGAC
AAGCTTATTGCTCGTAAAAAAGACTGGGATCCAAAAAAATATGGTGGTTTTGATAGT
CCAACGGTAGCTTATTCAGTCCTAGTGGTTGCTAAGGTGGAAAAAGGGAAATCGAA
GAAGTTAAAATCCGTTAAAGAGTTACTAGGGATCACAATTATGGAAAGAAGTTCCTT
TGAAAAAAATCCGATTGACTTTTTAGAAGCTAAAGGATATAAGGAAGTTAAAAAAG
ACTTAATCATTAAACTACCTAAATATAGTCTTTTTGAGTTAGAAAACGGTCGTAAAC
GGATGCTGGCTAGTGCCGGAGAATTACAAAAAGGAAATGAGCTGGCTCTGCCAAGC
AAATATGTGAATTTTTTATATTTAGCTAGTCATTATGAAAAGTTGAAGGGTAGTCCA
GAAGATAACGAACAAAAACAATTGTTTGTGGAGCAGCATAAGCATTATTTAGATGA
GATTATTGAGCAAATCAGTGAATTTTCTAAGCGTGTTATTTTAGCAGATGCCAATTT
AGATAAAGTTCTTAGTGCATATAACAAACATAGAGACAAACCAATACGTGAACAAG
CAGAAAATATTATTCATTTATTTACGTTGACGAATCTTGGAGCTCCCGCTGCTTTTAA
ATATTTTGATACAACAATTGATCGTAAACGATATACGTCTACAAAAGAAGTTTTAGA
TGCCACTCTTATCCATCAATCCATCACTGGTCTTTATGAAACACGCATTGATTTGAGT
CAGCTAGGAGGTGACTGA
MDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADLFL
AAKNLSDAILLSDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQL
LNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKK
DWDPKKYGGFDSPTVAYSVLVVAKVEKGKSKKLKSVKELLGITIMERSSFEKNPIDFLE
AKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGD
(single underline: HNH domain; double underline: RuvC domain)

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria. meningitidis (NCBI Ref: YP_002342100.1) or to a Cas9 from any other organism.

In some embodiments, dCas9 corresponds to, or comprises in part or in whole, a Cas9 amino acid sequence having one or more mutations that inactivate the Cas9 nuclease activity. For example, in some embodiments, a dCas9 domain comprises D10A and an H840A mutation or corresponding mutations in another Cas9. In some embodiments, the dCas9 comprises the amino acid sequence of dCas9 (D10A and H840A):

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
(single underline: HNH domain; double underline:
RuvC domain).

In some embodiments, the Cas9 domain comprises a D10A mutation, while the residue at position 840 remains a histidine in the amino acid sequence provided above, or at corresponding positions in any of the amino acid sequences provided herein.

In other embodiments, dCas9 variants having mutations other than D10A and H840A are provided, which, e.g., result in nuclease inactivated Cas9 (dCas9). Such mutations, by way of example, include other amino acid substitutions at D10 and H840, or other substitutions within the nuclease domains of Cas9 (e.g., substitutions in the HNH nuclease subdomain and/or the RuvC1 subdomain).

In some embodiments, variants or homologues of dCas9 are provided which are at least about 70% identical, at least about 80% identical, at least about 90% identical, at least about 95% identical, at least about 98% identical, at least about 99% identical, at least about 99.5% identical, or at least about 99.9% identical. In some embodiments, variants of dCas9 are provided having amino acid sequences which are shorter, or longer, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments, Cas9 fusion proteins as provided herein comprise the full-length amino acid sequence of a Cas9 protein, e.g., one of the Cas9 sequences provided herein. In other embodiments, however, fusion proteins as provided herein do not comprise a full-length Cas9 sequence, but only a fragment thereof. For example, in some embodiments, a Cas9 fusion protein provided herein comprises a Cas9 fragment, wherein the fragment binds crRNA and tracrRNA or sgRNA, but does not comprise a functional nuclease domain, e.g., in that it comprises only a truncated version of a nuclease domain or no nuclease domain at all.

Exemplary amino acid sequences of suitable Cas9 domains and Cas9 fragments are provided herein, and additional suitable sequences of Cas9 domains and fragments will be apparent to those of skill in the art.

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1).

It should be appreciated that additional Cas9 proteins (e.g., a nuclease dead Cas9 (dCas9), a Cas9 nickase (nCas9), or a nuclease active Cas9), including variants and homologs thereof, are within the scope of this disclosure. Exemplary Cas9 proteins include, without limitation, those provided below. In some embodiments, the Cas9 protein is a nuclease dead Cas9 (dCas9). In some embodiments, the Cas9 protein is a Cas9 nickase (nCas9). In some embodiments, the Cas9 protein is a nuclease active Cas9.

Exemplary catalytically inactive Cas9 (dCas9):

DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

Exemplary catalytically Cas9 nickase (nCas9):

DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

Exemplary catalytically active Cas9:

DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD.

In some embodiments, Cas9 refers to a Cas9 from archaea (e.g. nanoarchaea), which constitute a domain and kingdom of single-celled prokaryotic microbes. In some embodiments, Cas9 refers to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure.

In some embodiments, the nucleic acid programmable DNA binding protein (napDNAbp) or any of the fusion proteins provided herein may be a CasX or CasY protein. In some embodiments, the napDNAbp is a CasX protein. In some embodiments, the napDNAbp is a CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring CasX or CasY protein. In some embodiments, the napDNAbp is a naturally-occurring CasX or CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any CasX or CasY protein described herein. It should be appreciated that CasX and CasY from other bacterial species may also be used in accordance with the present disclosure.

CasX (uniprot.org/uniprot/FONN87; uniprot.org/uniprot/FONH53)

>tr|F0NN87|F0NN87_SULIH CRISPR-associated Casx protein OS=Sulfolobus islandicus (strain HVE10/4) GN=SiH_0402 PE=4 SV=1

MEVPLYNIFGDNYIIQVATEAENSTIYNNKVEIDDEELRNVLNLAYKIAK
NNEDAAAERRGKAKKKKGEEGETTTSNIILPLSGNDKNPWTETLKCYNFP
TTVALSEVFKNFSQVKECEEVSAPSFVKPEFYEFGRSPGMVERTRRVKLE
VEPHYLIIAAAGWVLTRLGKAKVSEGDYVGVNVFTPTRGILYSLIQNVNG
IVPGIKPETAFGLWIARKVVSSVTNPNVSVVRIYTISDAVGQNPTTINGG
FSIDLTKLLEKRYLLSERLEAIARNALSISSNMRERYIVLANYIYEYLTG
SKRLEDLLYFANRDLIMNLNSDDGKVRDLKLISAYVNGELIRGEG

>tr|F0NH₅₃|F0NH₅₃_SULIR CRISPR associated protein, Casx OS=Sulfolobus islandicus (strain REY15A) GN=SiRe_0771 PE=4 SV=1

MEVPLYNIFGDNYIIQVATEAENSTIYNNKVEIDDEELRNVLNLAYKIAK
NNEDAAAERRGKAKKKKGEEGETTTSNIILPLSGNDKNPWTETLKCYNFP
TTVALSEVFKNFSQVKECEEVSAPSFVKPEFYKFGRSPGMVERTRRVKLE
VEPHYLIMAAAGWVLTRLGKAKVSEGDYVGVNVFTPTRGILYSLIQNVNG
IVPGIKPETAFGLWIARKVVSSVTNPNVSVVSIYTISDAVGQNPTTINGG
FSIDLTKLLEKRDLLSERLEAIARNALSISSNMRERYIVLANYIYEYLTG
SKRLEDLLYFANRDLIMNLNSDDGKVRDLKLISAYVNGELIRGEG

CasY (ncbi.nlm.nih.gov/protein/APG80656.1)

>APG80656.1 CRISPR-associated protein CasY [uncultured Parcubacteria group bacterium]

MSKRHPRISGVKGYRLHAQRLEYTGKSGAMRTIKYPLYSSPSGGRTVPRE
IVSAINDDYVGLYGLSNFDDLYNAEKRNEEKVYSVLDFWYDCVQYGAVFS
YTAPGLLKNVAEVRGGSYELTKTLKGSHLYDELQIDKVIKFLNKKEISRA
NGSLDKLKKDIIDCFKAEYRERHKDQCNKLADDIKNAKKDAGASLGERQK
KLFRDFFGISEQSENDKPSFTNPLNLTCCLLPFDTVNNNRNRGEVLFNKL
KEYAQKLDKNEGSLEMWEYIGIGNSGTAFSNFLGEGFLGRLRENKITELK
KAMMDITDAWRGQEQEEELEKRLRILAALTIKLREPKFDNHWGGYRSDIN
GKLSSWLQNYINQTVKIKEDLKGHKKDLKKAKEMINRFGESDTKEEAVVS
SLLESIEKIVPDDSADDEKPDIPAIAIYRRFLSDGRLTLNRFVQREDVQE
ALIKERLEAEKKKKPKKRKKKSDAEDEKETIDFKELFPHLAKPLKLVPNF
YGDSKRELYKKYKNAAIYTDALWKAVEKIYKSAFSSSLKNSFFDTDFDKD
FFIKRLQKIFSVYRRFNTDKWKPIVKNSFAPYCDIVSLAENEVLYKPKQS
RSRKSAAIDKNRVRLPSTENIAKAGIALARELSVAGFDWKDLLKKEEHEE
YIDLIELHKTALALLLAVTETQLDISALDFVENGTVKDFMKTRDGNLVLE
GRFLEMFSQSIVFSELRGLAGLMSRKEFITRSAIQTMNGKQAELLYIPHE
FQSAKITTPKEMSRAFLDLAPAEFATSLEPESLSEKSLLKLKQMRYYPHY
FGYELTRTGQGIDGGVAENALRLEKSPVKKREIKCKQYKTLGRGQNKIVL
YVRSSYYQTQFLEWFLHRPKNVQTDVAVSGSFLIDEKKVKTRWNYDALTV
ALEPVSGSERVFVSQPFTIFPEKSAEEEGQRYLGIDIGEYGIAYTALEIT
GDSAKILDQNFISDPQLKTLREEVKGLKLDQRRGTFAMPSTKIARIRESL
VHSLRNRIHHLALKHKAKIVYELEVSRFEEGKQKIKKVYATLKKADVYSE
IDADKNLQTTVWGKLAVASEISASYTSQFCGACKKLWRAEMQVDETITTQ
ELIGTVRVIKGGTLIDAIKDFMRPPIFDENDTPFPKYRDFCDKHHISKKM
RGNSCLFICPFCRANADADIQASQTIALLRYVKEEKKVEDYFERFRKLKN
IKVLGQMKKI

The term “Cas12b” or “Cas12b domain” refers to an RNA-guided nuclease comprising a Cas12b/C2c1 protein, or a fragment thereof (e.g., a protein comprising an active, inactive, or partially active DNA cleavage domain of Cas12b, and/or the gRNA binding domain of Cas12b). contents of each of which are incorporated herein by reference). Cas12b orthologs have been described in various species, including, but not limited to, Alicyclobacillus acidoterrestris, Alicyclobacillus acidophilus (Teng et al., Cell Discov. 2018 Nov. 27; 4:63), Bacillus hisashi, and Bacillus sp. V3-13. Additional suitable Cas12b nucleases and sequences will be apparent to those of skill in the art based on this disclosure.

In some embodiments, proteins comprising Cas12b or fragments thereof are referred to as “Cas12b variants.” A Cas12b variant shares homology to Cas12b, or a fragment thereof. For example, a Cas12b variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas12b. In some embodiments, the Cas12b variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to wild type Cas12b. In some embodiments, the Cas12b variant comprises a fragment of Cas12b (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas12b. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas12b. Exemplary Cas12b polypeptides are listed below.

Cas12b/C2c1 (uniprot.org/uniprot/TOD7A2#2)

sp|TOD7A2├C2C1_ALIAG CRISPR-associated endo-nuclease C2c1 OS=Alicyclobacillus acido-terrestris (strain ATCC 49025/DSM 3922/CIP 106132/NCIMB 13137/GD3B) GN=c2c1 PE=1 SV=1

MAVKSIKVKLRLDDMPEIRAGLWKLHKEVNAGVRYYTEWLSLLRQENLYR
RSPNGDGEQECDKTAEECKAELLERLRARQVENGHRGPAGSDDELLQLAR
QLYELLVPQAIGAKGDAQQIARKFLSPLADKDAVGGLGIAKAGNKPRWVR
MREAGEPGWEEEKEKAETRKSADRTADVLRALADFGLKPLMRVYTDSEMS
SVEWKPLRKGQAVRTWDRDMFQQAIERMMSWESWNQRVGQEYAKLVEQKN
RFEQKNFVGQEHLVHLVNQLQQDMKEASPGLESKEQTAHYVTGRALRGSD
KVFEKWGKLAPDAPFDLYDAEIKNVQRRNTRRFGSHDLFAKLAEPEYQAL
WREDASFLTRYAVYNSILRKLNHAKMFATFTLPDATAHPIWTRFDKLGGN
LHQYTFLFNEFGERRHAIRFHKLLKVENGVAREVDDVTVPISMSEQLDNL
LPRDPNEPIALYFRDYGAEQHFTGEFGGAKIQCRRDQLAHMHRRRGARDV
YLNVSVRVQSQSEARGERRPPYAAVFRLVGDNHRAFVHFDKLSDYLAEHP
DDGKLGSEGLLSGLRVMSVDLGLRTSASISVFRVARKDELKPNSKGRVPF
FFPIKGNDNLVAVHERSQLLKLPGETESKDLRAIREERQRTLRQLRTQLA
YLRLLVRCGSEDVGRRERSWAKLIEQPVDAANHMTPDWREAFENELQKLK
SLHGICSDKEWMDAVYESVRRVWRHMGKQVRDWRKDVRSGERPKIRGYAK
DVVGGNSIEQIEYLERQYKFLKSWSFFGKVSGQVIRAEKGSRFAITLREH
IDHAKEDRLKKLADRIIMEALGYVYALDERGKGKWVAKYPPCQLILLEEL
SEYQFNNDRPPSENNQLMQWSHRGVFQELINQAQVHDLLVGTMYAAFSSR
FDARTGAPGIRCRRVPARCTQEHNPEPFPWWLNKFVVEHTLDACPLRADD
LIPTGEGEIFVSPFSAEEGDFHQIHADLNAAQNLQQRLWSDFDISQIRLR
CDWGEVDGELVLIPRLTGKRTADSYSNKVFYTNTGVTYYERERGKKRRKV
FAQEKLSEEEAELLVEADEAREKSVVLMRDPSGIINRGNWTRQKEFWSMV
NQRIEGYLVKQIRSRVPLQDSACENTGDI

AacCas12b (Alicyclobacillus acidiphilus)—WP_067623834

MAVKSMKVKLRLDNMPEIRAGLWKLHTEVNAGVRYYTEWLSLLRQENLYR
RSPNGDGEQECYKTAEECKAELLERLRARQVENGHCGPAGSDDELLQLAR
QLYELLVPQAIGAKGDAQQIARKFLSPLADKDAVGGLGIAKAGNKPRWVR
MREAGEPGWEEEKAKAEARKSTDRTADVLRALADFGLKPLMRVYTDSDMS
SVQWKPLRKGQAVRTWDRDMFQQAIERMMSWESWNQRVGEAYAKLVEQKS
RFEQKNFVGQEHLVQLVNQLQQDMKEASHGLESKEQTAHYLTGRALRGSD
KVFEKWEKLDPDAPFDLYDTEIKNVQRRNTRRFGSHDLFAKLAEPKYQAL
WREDASFLTRYAVYNSIVRKLNHAKMFATFTLPDATAHPIWTRFDKLGGN
LHQYTFLFNEFGEGRHAIRFQKLLTVEDGVAKEVDDVTVPISMSAQLDDL
LPRDPHELVALYFQDYGAEQHLAGEFGGAKIQYRRDQLNHLHARRGARDV
YLNLSVRVQSQSEARGERRPPYAAVFRLVGDNHRAFVHFDKLSDYLAEHP
DDGKLGSEGLLSGLRVMSVDLGLRTSASISVFRVARKDELKPNSEGRVPF
CFPIEGNENLVAVHERSQLLKLPGETESKDLRAIREERQRTLRQLRTQLA
YLRLLVRCGSEDVGRRERSWAKLIEQPMDANQMTPDWREAFEDELQKLKS
LYGICGDREWTEAVYESVRRVWRHMGKQVRDWRKDVRSGERPKIRGYQKD
VVGGNSIEQIEYLERQYKFLKSWSFFGKVSGQVIRAEKGSRFAITLREHI
DHAKEDRLKKLADRIIMEALGYVYALDDERGKGKWVAKYPPCQLILLEEL
SEYQFNNDRPPSENNQLMQWSHRGVFQELLNQAQVHDLLVGTMYAAFSSR
FDARTGAPGIRCRRVPARCAREQNPEPFPWWLNKFVAEHKLDGCPLRADD
LIPTGEGEFFVSPFSAEEGDFHQIHADLNAAQNLQRRLWSDFDISQIRLR
CDWGEVDGEPVLIPRTTGKRTADSYGNKVFYTKTGVTYYERERGKKRRKV
FAQEELSEEEAELLVEADEAREKSVVLMRDPSGIINRGDWTRQKEFWSMV
NQRIEGYLVKQIRSRVRLQESACENTGDI

BhCas12b (Bacillus hisashii) NCBI Reference Sequence: WP_095142515

MAPKKKRKVGIHGVPAAATRSFILKIEPNEEVKKGLWKTHEVLNHGIAYY
MNILKLIRQEAIYEHHEQDPKNPKKVSKAEIQAELWDFVLKMQKCNSFTH
EVDKDEVFNILRELYEELVPSSVEKKGEANQLSNKFLYPLVDPNSQSGKG
TASSGRKPRWYNLKIAGDPSWEEEKKKWEEDKKKDPLAKILGKLAEYGLI
PLFIPYTDSNEPIVKEIKWMEKSRNQSVRRLDKDMFIQALERFLSWESWN
LKVKEEYEKVEKEYKTLEERIKEDIQALKALEQYEKERQEQLLRDTLNTN
EYRLSKRGLRGWREIIQKWLKMDENEPSEKYLEVFKDYQRKHPREAGDYS
VYEFLSKKENHFIWRNHPEYPYLYATFCEIDKKKKDAKQQATFTLADPIN
HPLWVRFEERSGSNLNKYRILTEQLHTEKLKKKLTVQLDRLIYPTESGGW
EEKGKVDIVLLPSRQFYNQIFLDIEEKGKHAFTYKDESIKFPLKGTLGGA
RVQFDRDHLRRYPHKVESGNVGRIYFNMTVNIEPTESPVSKSLKIHRDDF
PKVVNFKPKELTEWIKDSKGKKLKSGIESLEIGLRVMSIDLGQRQAAAAS
IFEVVDQKPDIEGKLFFPIKGTELYAVHRASFNIKLPGETLVKSREVLRK
AREDNLKLMNQKLNFLRNVLHFQQFEDITEREKRVTKWISRQENSDVPLV
YQDELIQIRELMYKPYKDWVAFLKQLHKRLEVEIGKEVKHWRKSLSDGRK
GLYGISLKNIDEIDRTRKFLLRWSLRPTEPGEVRRLEPGQRFAIDQLNHL
NALKEDRLKKMANTIIMHALGYCYDVRKKKWQAKNPACQIILFEDLSNYN
PYEERSRFENSKLMKWSRREIPRQVALQGEIYGLQVGEVGAQFSSRFHAK
TGSPGIRCSVVTKEKLQDNRFFKNLQREGRLTLDKIAVLKEGDLYPDKGG
EKFISLSKDRKCVTTHADINAAQNLQKRFWTRTHGFYKVYCKAYQVDGQT
VYIPESKDQKQKIIEEFGEGYFILKDGVYEWVNAGKLKIKKGSSKQSSSE
LVDSDILKDSFDLASELKGEKLMLYRDPSGNVFPSDKWMAAGVFFGKLER
ILISKLTNQYSISTIEDDSSKQSMKRPAATKKAGQAKKKK

including the variant termed BvCas12b V4 (S893R/K846R/E837G changes rel. to wt above)

BvCas12b (Bacillus sp. V3-13) NCBI Reference Sequence: WP_101661451.1

MAIRSIKLKMKTNSGTDSIYLRKALWRTHQLINEGIAYYMNLLTLYRQEA
IGDKTKEAYQAELINIIRNQQRNNGSSEEHGSDQEILALLRQLYELIIPS
SIGESGDANQLGNKFLYPLVDPNSQSGKGTSNAGRKPRWKRLKEEGNPDW
ELEKKKDEERKAKDPTVKIFDNLNKYGLLPLFPLFTNIQKDIEWLPLGKR
QSVRKWDKDMFIQAIERLLSWESWNRRVADEYKQLKEKTESYYKEHLTGG
EEWIEKIRKFEKERNMELEKNAFAPNDGYFITSRQIRGWDRVYEKWSKLP
ESASPEELWKVVAEQQNKMSEGFGDPKVFSFLANRENRDIWRGHSERIYH
IAAYNGLQKKLSRTKEQATFTLPDAIEHPLWIRYESPGGTNLNLFKLEEK
QKKNYYVTLSKIIWPSEEKWIEKENIEIPLAPSIQFNRQIKLKQHVKGKQ
EISFSDYSSRISLDGVLGGSRIQFNRKYIKNHKELLGEGDIGPVFFNLVV
DVAPLQETRNGRLQSPIGKALKVISSDFSKVIDYKPKELMDWMNTGSASN
SFGVASLLEGMRVMSIDMGQRTSASVSIFEVVKELPKDQEQKLFYSINDT
ELFAIHKRSFLLNLPGEVVTKNNKQQRQERRKKRQFVRSQIRMLANVLRL
ETKKTPDERKKAIHKLMEIVQSYDSWTASQKEVWEKELNLLTNMAAFNDE
IWKESLVELHHRIEPYVGQIVSKWRKGLSEGRKNLAGISMWNIDELEDTR
RLLISWSKRSRTPGEANRIETDEPFGSSLLQHIQNVKDDRLKQMANLIIM
TALGFKYDKEEKDRYKRWKETYPACQIILFENLNRYLFNLDRSRRENSRL
MKWAHRSIPRTVSMQGEMFGLQVGDVRSEYSSRFHAKTGAPGIRCHALTE
EDLKAGSNTLKRLIEDGFINESELAYLKKGDIIPSQGGELFVTLSKRYKK
DSDNNELTVIHADINAAQNLQKRFWQQNSEVYRVPCQLARMGEDKLYIPK
SQTETIKKYFGKGSFVKNNTEQEVYKWEKSEKMKIKTDTTFDLQDLDGFE
DISKTIELAQEQQKKYLTMFRDPSGYFFNNETWRPQKEYWSIVNNIIKSC
LKKKILSNKVEL

By “Cbl proto-oncogene B (CBLB) polypeptide” is meant a protein having at least about 85% amino acid sequence identity to GenBank Accession No. ABC86700.1 or a fragment thereof that is involved in the regulation of immune responses. An exemplary CBLB polypeptide sequence is provided below.

>ABC86700.1 CBL-B [Homo sapiens]

MANSMNGRNPGGRGGNPRKGRILGIIDAIQDAVGPPKQAAADRRTVEKT
WKLMDKVVRLCQNPKLQLKNSPPYILDILPDTYQHLRLILSKYDDNQKL
AQLSENEYFKIYIDSLMKKSKRAIRLFKEGKERMYEEQSQDRRNLTKLS
LIFSHMLAEIKAIFPNGQFQGDNFRITKADAAEFWRKFFGDKTIVPWKV
FRQCLHEVHQISSGLEAMALKSTIDLTCNDYISVFEFDIFTRLFQPWGS
ILRNWNFLAVTHPGYMAFLTYDEVKARLQKYSTKPGSYIFRLSCTRLGQ
WAIGYVTGDGNILQTIPHNKPLFQALIDGSREGFYLYPDGRSYNPDLTG
LCEPTPHDHIKVTQEQYELYCEMGSTFQLCKICAENDKDVKIEPCGHLM
CTSCLTAWQESDGQGCPFCRCEIKGTEPIIVDPFDPRDEGSRCCSIIDP
FGMPMLDLDDDDDREESLMMNRLANVRKCTDRQNSPVTSPGSSPLAQRR
KPQPDPLQIPHLSLPPVPPRLDLIQKGIVRSPCGSPTGSPKSSPCMVRK
QDKPLPAPPPPLRDPPPPPPERPPPIPPDNRLSRHIHHVESVPSRDPPM
PLEAWCPRDVFGTNQLVGCRLLGEGSPKPGITASSNVNGRHSRVGSDPV
LMRKHRRHDLPLEGAKVFSNGHLGSEEYDVPPRLSPPPPVTTLLPSIKC
TGPLANSLSEKTRDPVEEDDDEYKIPSSHPVSLNSQPSHCHNVKPPVRS
CDNGHCMLNGTHGPSSEKKSNIPDLSIYLKGDVFDSASDPVPLPPARPP
TRDNPKHGSSLNRTPSDYDLLIPPLGEDAFDALPPSLPPPPPPARHSLI
EHSKPPGSSSRPSSGQDLFLLPSDPFVDLASGQVPLPPARRLPGENVKT
NRTSQDYDQLPSCSDGSQAPARPPKPRPRRTAPEIHHRKPHGPEAALEN
VDAKIAKLMGEGYAFEEVKRALEIAQNNVEVARSILREFAFPPPVSPRL
NL

By “Cbl proto-oncogene B (CBLB) polynucleotide” is meant a nucleic acid molecule encoding a CBLB polypeptide. The CBLB gene encodes an E3 ubiquitin ligase. An exemplary CBLB nucleic acid sequence is provided below. Additional exemplary CBLB genomic sequences are indicated in NCBI Reference Sequence: NC_000003.12, or transcript reference NM_001321813.1.

>DQ349203.1 Homo sapiens CBL-B mRNA, complete cds

ATGGCAAACTCAATGAATGGCAGAAACCCTGGTGGTCGAGGAGGAAATC
CCCGAAAAGGTCGAATTTTGGGTATTATTGATGCTATTCAGGATGCAGT
TGGACCCCCTAAGCAAGCTGCCGCAGATCGCAGGACCGTGGAGAAGACT
TGGAAGCTCATGGACAAAGTGGTAAGACTGTGCCAAAATCCCAAACTTC
AGTTGAAAAATAGCCCACCATATATACTTGATATTTTGCCTGATACATA
TCAGCATTTACGACTTATATTGAGTAAATATGATGACAACCAGAAACTT
GCCCAACTCAGTGAGAATGAGTACTTTAAAATCTACATTGATAGCCTTA
TGAAAAAGTCAAAACGGGCAATAAGACTCTTTAAAGAAGGCAAGGAGAG
AATGTATGAAGAACAGTCACAGGACAGACGAAATCTCACAAAACTGTCC
CTTATCTTCAGTCACATGCTGGCAGAAATCAAAGCAATCTTTCCCAATG
GTCAATTCCAGGGAGATAACTTTCGTATCACAAAAGCAGATGCTGCTGA
ATTCTGGAGAAAGTTTTTTGGAGACAAAACTATCGTACCATGGAAAGTA
TTCAGACAGTGCCTTCATGAGGTCCACCAGATTAGCTCTGGCCTGGAAG
CAATGGCTCTAAAATCAACAATTGATTTAACTTGCAATGATTACATTTC
AGTTTTTGAATTTGATATTTTTACCAGGCTGTTTCAGCCTTGGGGCTCT
ATTTTGCGGAATTGGAATTTCTTAGCTGTGACACATCCAGGTTACATGG
CATTTCTCACATATGATGAAGTTAAAGCACGACTACAGAAATATAGCAC
CAAACCCGGAAGCTATATTTTCCGGTTAAGTTGCACTCGATTGGGACAG
TGGGCCATTGGCTATGTGACTGGGGATGGGAATATCTTACAGACCATAC
CTCATAACAAGCCCTTATTTCAAGCCCTGATTGATGGCAGCAGGGAAGG
ATTTTATCTTTATCCTGATGGGAGGAGTTATAATCCTGATTTAACTGGA
TTATGTGAACCTACACCTCATGACCATATAAAAGTTACACAGGAACAAT
ATGAATTATATTGTGAAATGGGCTCCACTTTTCAGCTCTGTAAGATTTG
TGCAGAGAATGACAAAGATGTCAAGATTGAGCCTTGTGGGCATTTGATG
TGCACCTCTTGCCTTACGGCATGGCAGGAGTCGGATGGTCAGGGCTGCC
CTTTCTGTCGTTGTGAAATAAAAGGAACTGAGCCCATAATCGTGGACCC
CTTTGATCCAAGAGATGAAGGCTCCAGGTGTTGCAGCATCATTGACCCC
TTTGGCATGCCGATGCTAGACTTGGACGACGATGATGATCGTGAGGAGT
CCTTGATGATGAATCGGTTGGCAAACGTCCGAAAGTGCACTGACAGGCA
GAACTCACCAGTCACATCACCAGGATCCTCTCCCCTTGCCCAGAGAAGA
AAGCCACAGCCTGACCCACTCCAGATCCCACATCTAAGCCTGCCACCCG
TGCCTCCTCGCCTGGATCTAATTCAGAAAGGCATAGTTAGATCTCCCTG
TGGCAGCCCAACGGGTTCACCAAAGTCTTCTCCTTGCATGGTGAGAAAA
CAAGATAAACCACTCCCAGCACCACCTCCTCCCTTAAGAGATCCTCCTC
CACCGCCACCTGAAAGACCTCCACCAATCCCACCAGACAATAGACTGAG
TAGACACATCCATCATGTGGAAAGCGTGCCTTCCAGAGACCCGCCAATG
CCTCTTGAAGCATGGTGCCCTCGGGATGTGTTTGGGACTAATCAGCTTG
TGGGATGTCGACTCCTAGGGGAGGGCTCTCCAAAACCTGGAATCACAGC
GAGTTCAAATGTCAATGGAAGGCACAGTAGAGTGGGCTCTGACCCAGTG
CTTATGCGGAAACACAGACGCCATGATTTGCCTTTAGAAGGAGCTAAGG
TCTTTTCCAATGGTCACCTTGGAAGTGAAGAATATGATGTTCCTCCCCG
GCTTTCTCCTCCTCCTCCAGTTACCACCCTCCTCCCTAGCATAAAGTGT
ACTGGTCCGTTAGCAAATTCTCTTTCAGAGAAAACAAGAGACCCAGTAG
AGGAAGATGATGATGAATACAAGATTCCTTCATCCCACCCTGTTTCCCT
GAATTCACAACCATCTCATTGTCATAATGTAAAACCTCCTGTTCGGTCT
TGTGATAATGGTCACTGTATGCTGAATGGAACACATGGTCCATCTTCAG
AGAAGAAATCAAACATCCCTGACTTAAGCATATATTTAAAGGGAGATGT
TTTTGATTCAGCCTCTGATCCCGTGCCATTACCACCTGCCAGGCCTCCA
ACTCGGGACAATCCAAAGCATGGTTCTTCACTCAACAGGACGCCCTCTG
ATTATGATCTTCTCATCCCTCCATTAGGTGAAGATGCTTTTGATGCCCT
CCCTCCATCTCTCCCACCTCCCCCACCTCCTGCAAGGCATAGTCTCATT
GAACATTCAAAACCTCCTGGCTCCAGTAGCCGGCCATCCTCAGGACAGG
ATCTTTTTCTTCTTCCTTCAGATCCCTTTGTTGATCTAGCAAGTGGCCA
AGTTCCTTTGCCTCCTGCTAGAAGGTTACCAGGTGAAAATGTCAAAACT
AACAGAACATCACAGGACTATGATCAGCTTCCTTCATGTTCAGATGGTT
CACAGGCACCAGCCAGACCCCCTAAACCACGACCGCGCAGGACTGCACC
AGAAATTCACCACAGAAAACCCCATGGGCCTGAGGCGGCATTGGAAAAT
GTCGATGCAAAAATTGCAAAACTCATGGGAGAGGGTTATGCCTTTGAAG
AGGTGAAGAGAGCCTTAGAGATAGCCCAGAATAATGTCGAAGTTGCCCG
GAGCATCCTCCGAGAATTTGCCTTCCCTCCTCCAGTATCCCCACGTCTA
AATCTATAG

By “chimeric antigen receptor” is meant a synthetic receptor comprising an extracellular antigen binding domain, a transmembrane domain, and an intracellular signaling domain that confers specificity for an antigen onto an immune cell.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “cluster of differentiation 2 (CD2)” is meant a protein having at least about 85% amino acid sequence identity to NCBI Accession No. NP_001315538.1 or fragment thereof and having immunomodulatory activity. An exemplary amino acid sequence is provided below.

>NP_001315538.1 T-cell surface antigen CD2 isoform 1 precursor [Homo sapiens]

MSFPCKFVASFLLIFNVSSKGAVSKEITNALETWGALGQDINLDIPSFQ
MSDDIDDIKWEKTSDKKKIAQFRKEKETFKEKDTYKLFKNGTLKIKHLK
TDDQDIYKVSIYDTKGKNVLEKIFDLKIQERVSKPKISWTCINTTLTCE
VMNGTDPELNLYQDGKHLKLSQRVITHKWTTSLSAKFKCTAGNKVSKES
SVEPVSCPGGSILGQSNGLSAWTPPSHPTSLPFAEKGLDIYLIIGICGG
GSLLMVFVALLVFYITKRKKQRSRRNDEELETRAHRVATEERGRKPHQI
PASTPQNPATSQHPPPPPGHRSQAPSHRPPPPGHRVQHQPQKRPPAPSG
TQVHQQKGPPLPRPRVQPKPPHGAAENSLSPSSN

By “cluster of differentiation 2 (CD2)” is meant a nucleic acid encoding a CD2 polypeptide. An exemplary CD2 nucleic acid sequence is provided below. >NM_001328609.2 Homo sapiens CD2 molecule (CD2), transcript variant 1, mRNA

AGTCTCACTTCAGTTCCTTTTGCATGAAGAGCTCAGAATCAAAAGAGG
AAACCAACCCCTAAGATGAGCTTTCCATGTAAATTTGTAGCCAGCTTC
CTTCTGATTTTCAATGTTTCTTCCAAAGGTGCAGTCTCCAAAGAGATT
ACGAATGCCTTGGAAACCTGGGGTGCCTTGGGTCAGGACATCAACTTG
GACATTCCTAGTTTTCAAATGAGTGATGATATTGACGATATAAAATGG
GAAAAAACTTCAGACAAGAAAAAGATTGCACAATTCAGAAAAGAGAAA
GAGACTTTCAAGGAAAAAGATACATATAAGCTATTTAAAAATGGAACT
CTGAAAATTAAGCATCTGAAGACCGATGATCAGGATATCTACAAGGTA
TCAATATATGATACAAAAGGAAAAAATGTGTTGGAAAAAATATTTGAT
TTGAAGATTCAAGAGAGGGTCTCAAAACCAAAGATCTCCTGGACTTGT
ATCAACACAACCCTGACCTGTGAGGTAATGAATGGAACTGACCCCGAA
TTAAACCTGTATCAAGATGGGAAACATCTAAAACTTTCTCAGAGGGTC
ATCACACACAAGTGGACCACCAGCCTGAGTGCAAAATTCAAGTGCACA
GCAGGGAACAAAGTCAGCAAGGAATCCAGTGTCGAGCCTGTCAGCTGT
CCAGGAGGCAGCATCCTTGGCCAGAGTAATGGGCTCTCTGCCTGGACC
CCTCCCAGCCATCCCACTTCTCTTCCTTTTGCAGAGAAAGGTCTGGAC
ATCTATCTCATCATTGGCATATGTGGAGGAGGCAGCCTCTTGATGGTC
TTTGTGGCACTGCTCGTTTTCTATATCACCAAAAGGAAAAAACAGAGG
AGTCGGAGAAATGATGAGGAGCTGGAGACAAGAGCCCACAGAGTAGCT
ACTGAAGAAAGGGGCCGGAAGCCCCACCAAATTCCAGCTTCAACCCCT
CAGAATCCAGCAACTTCCCAACATCCTCCTCCACCACCTGGTCATCGT
TCCCAGGCACCTAGTCATCGTCCCCCGCCTCCTGGACACCGTGTTCAG
CACCAGCCTCAGAAGAGGCCTCCTGCTCCGTCGGGCACACAAGTTCAC
CAGCAGAAAGGCCCGCCCCTCCCCAGACCTCGAGTTCAGCCAAAACCT
CCCCATGGGGCAGCAGAAAACTCATTGTCCCCTTCCTCTAATTAAAAA
AGATAGAAACTGTCTTTTTCAATAAAAAGCACTGTGGATTTCTGCCCT
CCTGATGTGCATATCCGTACTTCCATGAGGTGTTTTCTGTGTGCAGAA
CATTGTCACCTCCTGAGGCTGTGGGCCACAGCCACCTCTGCATCTTCG
AACTCAGCCATGTGGTCAACATCTGGAGTTTTTGGTCTCCTCAGAGAG
CTCCATCACACCAGTAAGGAGAAGCAATATAAGTGTGATTGCAAGAAT
GGTAGAGGACCGAGCACAGAAATCTTAGAGATTTCTTGTCCCCTCTCA
GGTCATGTGTAGATGCGATAAATCAAGTGATTGGTGTGCCTGGGTCTC
ACTACAAGCAGCCTATCTGCTTAAGAGACTCTGGAGTTTCTTATGTGC
CCTGGTGGACACTTGCCCACCATCCTGTGAGTAAAAGTGAAATAAAAG
CTTTGACTAGA

By “cluster of differentiation 3 epsilon (CD3e or CD3 epsilon)” is meant a protein having at least about 85% amino acid sequence identity to NCBI Accession No. NP_000724.1 or fragment thereof and having immunomodulatory activity. An exemplary amino acid sequence is provided below.

>NP_000724.1 T-cell surface glycoprotein CD3 epsilon chain precursor [Homo sapiens]

MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI

By “cluster of differentiation 3 epsilon (CD3e or CD3 epsilon)” is meant a nucleic acid encoding a CD3e polypeptide. An exemplary CD3e nucleic acid sequence is provided below.

>NM_000733.4 Homo sapiens CD3e molecule (CD3E), mRNA

AGAAACCCTCCTCCCCTCCCAGCCTCAGGTGCCTGCTTCAGAAAATGAA
GTAGTAAGTCTGCTGGCCTCCGCCATCTTAGTAAAGTAACAGTCCCATG
AAACAAAGATGCAGTCGGGCACTCACTGGAGAGTTCTGGGCCTCTGCCT
CTTATCAGTTGGCGTTTGGGGGCAAGATGGTAATGAAGAAATGGGTGGT
ATTACACAGACACCATATAAAGTCTCCATCTCTGGAACCACAGTAATAT
TGACATGCCCTCAGTATCCTGGATCTGAAATACTATGGCAACACAATGA
TAAAAACATAGGCGGTGATGAGGATGATAAAAACATAGGCAGTGATGAG
GATCACCTGTCACTGAAGGAATTTTCAGAATTGGAGCAAAGTGGTTATT
ATGTCTGCTACCCCAGAGGAAGCAAACCAGAAGATGCGAACTTTTATCT
CTACCTGAGGGCAAGAGTGTGTGAGAACTGCATGGAGATGGATGTGATG
TCGGTGGCCACAATTGTCATAGTGGACATCTGCATCACTGGGGGCTTGC
TGCTGCTGGTTTACTACTGGAGCAAGAATAGAAAGGCCAAGGCCAAGCC
TGTGACACGAGGAGCGGGTGCTGGCGGCAGGCAAAGGGGACAAAACAAG
GAGAGGCCACCACCTGTTCCCAACCCAGACTATGAGCCCATCCGGAAAG
GCCAGCGGGACCTGTATTCTGGCCTGAATCAGAGACGCATCTGACCCTC
TGGAGAACACTGCCTCCCGCTGGCCCAGGTCTCCTCTCCAGTCCCCCTG
CGACTCCCTGTTTCCTGGGCTAGTCTTGGACCCCACGAGAGAGAATCGT
TCCTCAGCCTCATGGTGAACTCGCGCCCTCCAGCCTGATCCCCCGCTCC
CTCCTCCCTGCCTTCTCTGCTGGTACCCAGTCCTAAAATATTGCTGCTT
CCTCTTCCTTTGAAGCATCATCAGTAGTCACACCCTCACAGCTGGCCTG
CCCTCTTGCCAGGATATTTATTTGTGCTATTCACTCCCTTCCCTTTGGA
TGTAACTTCTCCGTTCAGTTCCCTCCTTTTCTTGCATGTAAGTTGTCCC
CCATCCCAAAGTATTCCATCTACTTTTCTATCGCCGTCCCCTTTTGCAG
CCCTCTCTGGGGATGGACTGGGTAAATGTTGACAGAGGCCCTGCCCCGT
TCACAGATCCTGGCCCTGAGCCAGCCCTGTGCTCCTCCCTCCCCCAACA
CTCCCTACCAACCCCCTAATCCCCTACTCCCTCCACCCCCCCTCCACTG
TAGGCCACTGGATGGTCATTTGCATCTCCGTAAATGTGCTCTGCTCCTC
AGCTGAGAGAGAAAAAAATAAACTGTATTTGGCTGCAA

By “cluster of differentiation 3 gamma (CD3g or CD3 gamma) is meant a protein having at least about 85% amino acid sequence identity to NCBI Accession No. NP_000064.1 or fragment thereof and having immunomodulatory activity. An exemplary amino acid sequence is provided below.

>NP_000064.1 T-cell surface glycoprotein CD3 gamma chain precursor [Homo sapiens]

MEQGKGLAVLILAIILLQGTLAQSIKGNHLVKVYDYQEDGSVLLTCDAE
AKNITWFKDGKMIGFLTEDKKKWNLGSNAKDPRGMYQCKGSQNKSKPLQ
VYYRMCQNCIELNAATISGFLFAEIVSIFVLAVGVYFIAGQDGVRQSRA
SDKQTLLPNDQLYQPLKDREDDQYSHLQGNQLRRN

By “cluster of differentiation 3 gamma (CD3g or CD3 gamma)” is meant a nucleic acid encoding a CD3g polypeptide. An exemplary CD3g nucleic acid sequence is provided below.

>NM 000073.3 Homo sapiens CD3g molecule (CD3G), mRNA

AGTCTAGCTGCTGCACAGGCTGGCTGGCTGGCTGGCTGCTAAGGGCTGC
TCCACGCTTTTGCCGGAGGACAGAGACTGACATGGAACAGGGGAAGGGC
CTGGCTGTCCTCATCCTGGCTATCATTCTTCTTCAAGGTACTTTGGCCC
AGTCAATCAAAGGAAACCACTTGGTTAAGGTGTATGACTATCAAGAAGA
TGGTTCGGTACTTCTGACTTGTGATGCAGAAGCCAAAAATATCACATGG
TTTAAAGATGGGAAGATGATCGGCTTCCTAACTGAAGATAAAAAAAAAT
GGAATCTGGGAAGTAATGCCAAGGACCCTCGAGGGATGTATCAGTGTAA
AGGATCACAGAACAAGTCAAAACCACTCCAAGTGTATTACAGAATGTGT
CAGAACTGCATTGAACTAAATGCAGCCACCATATCTGGCTTTCTCTTTG
CTGAAATCGTCAGCATTTTCGTCCTTGCTGTTGGGGTCTACTTCATTGC
TGGACAGGATGGAGTTCGCCAGTCGAGAGCTTCAGACAAGCAGACTCTG
TTGCCCAATGACCAGCTCTACCAGCCCCTCAAGGATCGAGAAGATGACC
AGTACAGCCACCTTCAAGGAAACCAGTTGAGGAGGAATTGAACTCAGGA
CTCAGAGTAGTCCAGGTGTTCTCCTCCTATTCAGTTCCCAGAATCAAAG
CAATGCATTTTGGAAAGCTCCTAGCAGAGAGACTTTCAGCCCTAAATCT
AGACTCAAGGTTCCCAGAGATGACAAATGGAGAAGAAAGGCCATCAGAG
CAAATTTGGGGGTTTCTCAAATAAAATAAAAATAAAAACAAATACTGTG
TTTCAGAAGCGCCACCTATTGGGGAAAATTGTAAAAGAAAAATGAAAAG
ATCAAATAACCCCCTGGATTTGAATATAATTTTTTGTGTTGTAATTTTT
ATTTCGTTTTTGTATAGGTTATAATTCACATGGCTCAAATATTCAGTGA
AAGCTCTCCCTCCACCGCCATCCCCTGCTACCCAGTGACCCTGTTGCCC
TCTTCAGAGACAAATTAGTTTCTCTTTTTTTTTTTTTTTTTTTTTTTTT
TGAGACAGTCTGGCTCTGTCACCCAGGCTGAAATGCAGTGGCACCATCT
CGGCTCACTGCAACCTCTGCCTCCTGGGTTCAAGCGATTCTCCTGCCTC
AGCCTCCCGGGCAGCTGGGATTACAGGCACACACTACCACACCTGGCTA
ATTTTTGTATTTTTAGTAGAGACAGGGTTTTGCTCTGTTGGCCAAGCTG
GTCTCGAACTCCTGACCTCAAGTGATCCGCCCGCCTCAGCCTCCCAAAG
TGCTGGGATTACAGGTGTGAGCCACCATGCCTGGTCTTAAAACCAGTTT
CTTATATATCTCTCTGGAGGTATTCTAGGCATATATGAGCACATTCTCA
AGTACATATTATCCTCCCTTCCCCTATCTTTTAGACAAATGATATCAAA
CTATACATCTTGTGAGATTATTGCATACCATTATATGAAGATACCATTA
TATCCTTTTTAATGCAACCATATTGTACAAATAGACTATGATTTATTTA
ACCTGTTATCTATCAGTGGATATTTAAGTTGGTAGTTGGTTCCAATCTT
TTGCTCTTACAACAATTCTGCAATGACTAACATTGTATAAATATCATTT
TTAAAAATAATTGCATTGAAGCATAATGTACATGCCATAAAATCCACCC
ATCTTAAGTGATTTCACCTGTTCTCAGAAATTTTTAGTAAATTTAACTA
ATTGTACAGCCATTACCATAATCCAGCTTTAGGACATTTTCTTTTTTTT
CTTTTCTTTTCTTTTTTTTCTTTTTTTTTTTTTTTTGAAGTGGAATCTT
GCTCTGTGGCCCAGGCTGGAGTGCAGTGGCGCGATCTCAGCTCACTGCA
ACCTCCACCTCCTGGGTTCAAGCGATTCTCTTGCCTTGGCCTCCCGAGT
AGCTGAGACTACAGGCACATGCCACCACGCCCAGCTCATTTTTTGTGTA
TTTAGTATTTGTGTATCTAGTATTTGTGTACTTAGTAGAGACAGGGTTT
CACCATGTTGGCCAGGCTGGTCTCCAATTCCTGACCTCAGGCGATCCAC
CCGCCTTGACCTCCCAAAGTGCTGGGATTACAGGTGTGAGCCACCGCGC
CAGGCCCGTAACTGTATTTTAATATAGCCATTCTATGGATTTAATATGG
TATTTTATTATGGCCTTAATTTGCATTTCCCTAGATACTAACCATGCTG
AGTGTCCTGTCTTGTGTTTATTAACCATTCATATATTTTTAGTGAAATG
TGTATCAAATCTTTTGCCCATTTTTAAGTTGACTTATTTGTTTGTCTTC
TTACTATTGGGTTGCATATGTTTTTGATATAAGTCCTTTATCAGATATA
TGATTTGGAAATATTTTCTACCAATCTGTGGTTTGTTTTTCTTAATGGT
GTCTTTTGAAGTGCAAAAGGTTTGAATTTTGAAGTACATTTTATTGATT
TTTTCTTCTATATATTGTGCTTTTGGTATCATGTCTAATAAATCTTTAC
CAAACCCACAGTTACAAAGATTTTCTCCTGTCTTCTTTTTATACTTTTT
ACAGCTTTATGGTTTTAGCTCTAACAATAAATGTGATTTTGAACATACA
TAAGACTATTTGTAACAAACACAAATAAATTGAATTGTTGGGCA

By “cluster of differentiation 3 delta (CD3d or CD3 delta) is meant a protein having at least about 85% amino acid sequence identity to NCBI Accession No. NP_000723.1 or fragment thereof and having immunomodulatory activity. An exemplary amino acid sequence is provided below.

>NP_000723.1 T-cell surface glycoprotein CD3 delta chain isoform A precursor [Homo sapiens]

MEHSTFLSGLVLATLLSQVSPFKIPIEELEDRVFVNCNTSITWVEGTVG
TLLSDITRLDLGKRILDPRGIYRCNGTDIYKDKESTVQVHYRMCQSCVE
LDPATVAGIIVTDVIATLLLALGVFCFAGHETGRLSGAADTQALLRNDQ
VYQPLRDRDDAQYSHLGGNWARNK

By “cluster of differentiation 3 delta (CD3d or CD3 delta)” is meant a nucleic acid encoding a CD3d polypeptide. An exemplary CD3d nucleic acid sequence is provided below.

>NM_000732.4 Homo sapiens CD3d molecule (CD3D), transcript variant 1, mRNA

AGAGAAGCAGACATCTTCTAGTTCCTCCCCCACTCTCCTCTTTCCGGTA
CCTGTGAGTCAGCTAGGGGAGGGCAGCTCTCACCCAGGCTGATAGTTCG
GTGACCTGGCTTTATCTACTGGATGAGTTCCGCTGGGAGATGGAACATA
GCACGTTTCTCTCTGGCCTGGTACTGGCTACCCTTCTCTCGCAAGTGAG
CCCCTTCAAGATACCTATAGAGGAACTTGAGGACAGAGTGTTTGTGAAT
TGCAATACCAGCATCACATGGGTAGAGGGAACGGTGGGAACACTGCTCT
CAGACATTACAAGACTGGACCTGGGAAAACGCATCCTGGACCCACGAGG
AATATATAGGTGTAATGGGACAGATATATACAAGGACAAAGAATCTACC
GTGCAAGTTCATTATCGAATGTGCCAGAGCTGTGTGGAGCTGGATCCAG
CCACCGTGGCTGGCATCATTGTCACTGATGTCATTGCCACTCTGCTCCT
TGCTTTGGGAGTCTTCTGCTTTGCTGGACATGAGACTGGAAGGCTGTCT
GGGGCTGCCGACACACAAGCTCTGTTGAGGAATGACCAGGTCTATCAGC
CCCTCCGAGATCGAGATGATGCTCAGTACAGCCACCTTGGAGGAAACTG
GGCTCGGAACAAGTGAACCTGAGACTGGTGGCTTCTAGAAGCAGCCATT
ACCAACTGTACCTTCCCTTCTTGCTCAGCCAATAAATATATCCTCTTTC
ACTCAGAAAAAAAAAAAAAAAAAAAAAAAAAAAAAA

By “cluster of differentiation 4 (CD4)” is meant a protein having at least about 85% amino acid sequence identity to NCBI Accession No. NP_000607.1 or fragment thereof and having immunomodulatory activity. An exemplary amino acid sequence is provided below.

>NP_000607.1 T-cell surface glycoprotein CD4 isoform 1 precursor [Homo sapiens]

MNRGVPFRHLLLVLQLALLPAATQGKKVVLGKKGDTVELTCTASQKKSI
QFHWKNSNQIKILGNQGSFLTKGPSKLNDRADSRRSLWDQGNFPLIIKN
LKIEDSDTYICEVEDQKEEVQLLVFGLTANSDTHLLQGQSLTLTLESPP
GSSPSVQCRSPRGKNIQGGKTLSVSQLELQDSGTWTCTVLQNQKKVEFK
IDIVVLAFQKASSIVYKKEGEQVEFSFPLAFTVEKLTGSGELWWQAERA
SSSKSWITFDLKNKEVSVKRVTQDPKLQMGKKLPLHLTLPQALPQYAGS
GNLTLALEAKTGKLHQEVNLVVMRATQLQKNLTCEVWGPTSPKLMLSLK
LENKEAKVSKREKAVWVLNPEAGMWQCLLSDSGQVLLESNIKVLPTWST
PVQPMALIVLGGVAGLLLFIGLGIFFCVRCRHRRRQAERMSQIKRLLSE
KKTCQCPHRFQKTCSPI

By “cluster of differentiation 4 (CD4)” is meant a nucleic acid encoding a CD4 polypeptide. An exemplary CD4 nucleic acid sequence is provided below.

>NM_000616.5 Homo sapiens CD4 molecule (CD4), transcript variant 1, mRNA

CTCTCTTCATTTAAGCACGACTCTGCAGAAGGAACAAAGCACCCTCCCC
ACTGGGCTCCTGGTTGCAGAGCTCCAAGTCCTCACACAGATACGCCTGT
TTGAGAAGCAGCGGGCAAGAAAGACGCAAGCCCAGAGGCCCTGCCATTT
CTGTGGGCTCAGGTCCCTACTGGCTCAGGCCCCTGCCTCCCTCGGCAAG
GCCACAATGAACCGGGGAGTCCCTTTTAGGCACTTGCTTCTGGTGCTGC
AACTGGCGCTCCTCCCAGCAGCCACTCAGGGAAAGAAAGTGGTGCTGGG
CAAAAAAGGGGATACAGTGGAACTGACCTGTACAGCTTCCCAGAAGAAG
AGCATACAATTCCACTGGAAAAACTCCAACCAGATAAAGATTCTGGGAA
ATCAGGGCTCCTTCTTAACTAAAGGTCCATCCAAGCTGAATGATCGCGC
TGACTCAAGAAGAAGCCTTTGGGACCAAGGAAACTTTCCCCTGATCATC
AAGAATCTTAAGATAGAAGACTCAGATACTTACATCTGTGAAGTGGAGG
ACCAGAAGGAGGAGGTGCAATTGCTAGTGTTCGGATTGACTGCCAACTC
TGACACCCACCTGCTTCAGGGGCAGAGCCTGACCCTGACCTTGGAGAGC
CCCCCTGGTAGTAGCCCCTCAGTGCAATGTAGGAGTCCAAGGGGTAAAA
ACATACAGGGGGGGAAGACCCTCTCCGTGTCTCAGCTGGAGCTCCAGGA
TAGTGGCACCTGGACATGCACTGTCTTGCAGAACCAGAAGAAGGTGGAG
TTCAAAATAGACATCGTGGTGCTAGCTTTCCAGAAGGCCTCCAGCATAG
TCTATAAGAAAGAGGGGGAACAGGTGGAGTTCTCCTTCCCACTCGCCTT
TACAGTTGAAAAGCTGACGGGCAGTGGCGAGCTGTGGTGGCAGGCGGAG
AGGGCTTCCTCCTCCAAGTCTTGGATCACCTTTGACCTGAAGAACAAGG
AAGTGTCTGTAAAACGGGTTACCCAGGACCCTAAGCTCCAGATGGGCAA
GAAGCTCCCGCTCCACCTCACCCTGCCCCAGGCCTTGCCTCAGTATGCT
GGCTCTGGAAACCTCACCCTGGCCCTTGAAGCGAAAACAGGAAAGTTGC
ATCAGGAAGTGAACCTGGTGGTGATGAGAGCCACTCAGCTCCAGAAAAA
TTTGACCTGTGAGGTGTGGGGACCCACCTCCCCTAAGCTGATGCTGAGT
TTGAAACTGGAGAACAAGGAGGCAAAGGTCTCGAAGCGGGAGAAGGCGG
TGTGGGTGCTGAACCCTGAGGCGGGGATGTGGCAGTGTCTGCTGAGTGA
CTCGGGACAGGTCCTGCTGGAATCCAACATCAAGGTTCTGCCCACATGG
TCCACCCCGGTGCAGCCAATGGCCCTGATTGTGCTGGGGGGCGTCGCCG
GCCTCCTGCTTTTCATTGGGCTAGGCATCTTCTTCTGTGTCAGGTGCCG
GCACCGAAGGCGCCAAGCAGAGCGGATGTCTCAGATCAAGAGACTCCTC
AGTGAGAAGAAGACCTGCCAGTGTCCTCACCGGTTTCAGAAGACATGTA
GCCCCATTTGAGGCACGAGGCCAGGCAGATCCCACTTGCAGCCTCCCCA
GGTGTCTGCCCCGCGTTTCCTGCCTGCGGACCAGATGAATGTAGCAGAT
CCCCAGCCTCTGGCCTCCTGTTCGCCTCCTCTACAATTTGCCATTGTTT
CTCCTGGGTTAGGCCCCGGCTTCACTGGTTGAGTGTTGCTCTCTAGTTT
CCAGAGGCTTAATCACACCGTCCTCCACGCCATTTCCTTTTCCTTCAAG
CCTAGCCCTTCTCTCATTATTTCTCTCTGACCCTCTCCCCACTGCTCAT
TTGGATCCCAGGGGAGTGTTCAGGGCCAGCCCTGGCTGGCATGGAGGGT
GAGGCTGGGTGTCTGGAAGCATGGAGCATGGGACTGTTCTTTTACAAGA
CAGGACCCTGGGACCACAGAGGGCAGGAACTTGCACAAAATCACACAGC
CAAGCCAGTCAAGGATGGATGCAGATCCAGAGGTTTCTGGCAGCCAGTA
CCTCCTGCCCCATGCTGCCCGCTTCTCACCCTATGTGGGTGGGACCACA
GACTCACATCCTGACCTTGCACAAACAGCCCCTCTGGACACAGCCCCAT
GTACACGGCCTCAAGGGATGTCTCACATCCTCTGTCTATTTGAGACTTA
GAAAAATCCTACAAGGCTGGCAGTGACAGAACTAAGATGATCATCTCCA
GTTTATAGACCAGAACCAGAGCTCAGAGAGGCTAGATGATTGATTACCA
AGTGCCGGACTAGCAAGTGCTGGAGTCGGGACTAACCCAGGTCCCTTGT
CCCAAGTTCCACTGCTGCCTCTTGAATGCAGGGACAAATGCCACACGGC
TCTCACCAGTGGCTAGTGGTGGGTACTCAATGTGTACTTTTGGGTTCAC
AGAAGCACAGCACCCATGGGAAGGGTCCATCTCAGAGAATTTACGAGCA
GGGATGAAGGCCTCCCTGTCTAAAATCCCTCCTTCATCCCCCGCTGGTG
GCAGAATCTGTTACCAGAGGACAAAGCCTTTGGCTCTTCTAATCAGAGC
GCAAGCTGGGAGCACAGGCACTGCAGGAGAGAATGCCCAGTGACCAGTC
ACTGACCCTGTGCAGAACCTCCTGGAAGCGAGCTTTGCTGGGAGAGGGG
GTAGCTAGCCTGAGAGGGAACCCTCTAAGGGACCTCAAAGGTGATTGTG
CCAGGCTCTGCGCCTGCCCCACACCCTCCCTTACCCTCCTCCAGACCAT
TCAGGACACAGGGAAATCAGGGTTACAAATCTTCTTGATCCACTTCTCT
CAGGATCCCCTCTCTTCCTACCCTTCCTCACCACTTCCCTCAGTCCCAA
CTCCTTTTCCCTATTTCCTTCTCCTCCTGTCTTTAAAGCCTGCCTCTTC
CAGGAAGACCCCCCTATTGCTGCTGGGGCTCCCCATTTGCTTACTTTGC
ATTTGTGCCCACTCTCCACCCCTGCTCCCCTGAGCTGAAATAAAAATAC
AATAAACTTAC

By “cluster of differentiation 5 (CD5)” is meant a protein having at least about 85% amino acid sequence identity to NCBI Accession No. NP_001333385.1 or fragment thereof and having immunomodulatory activity. An exemplary amino acid sequence is provided below.

>NP_001333385.1 T-cell surface glycoprotein CD5 isoform 2 [Homo sapiens]

MVCSQSWGRSSKQWEDPSQASKVCQRLNCGVPLSLGPFLVTYTPQSSII
CYGQLGSFSNCSHSRNDMCHSLGLTCLEPQKTTPPTTRPPPTTTPEPTA
PPRLQLVAQSGGQHCAGVVEFYSGSLGGTISYEAQDKTQDLENFLCNNL
QCGSFLKHLPETEAGRAQDPGEPREHQPLPIQWKIQNSSCTSLEHCFRK
IKPQKSGRVLALLCSGFQPKVQSRLVGGSSICEGTVEVRQGAQWAALCD
SSSARSSLRWEEVCREQQCGSVNSYRVLDAGDPTSRGLFCPHQKLSQCH
ELWERNSYCKKVFVTCQDPNPAGLAAGTVASIILALVLLVVLLVVCGPL
AYKKLVKKFRQKKQRQWIGPTGMNQNMSFHRNHTATVRSHAENPTASHV
DNEYSQPPRNSHLSAYPALEGALHRSSMQPDNSSDSDYDLHGAQRL

By “cluster of differentiation 5 (CD5)” is meant a nucleic acid encoding a CD5 polypeptide. An exemplary CD5 nucleic acid sequence is provided below. >NM_001346456.1 Homo sapiens CD5 molecule (CD5), transcript variant 2, mRNA

GAGTCTTGCTGATGCTCCCGGCTGAATAAACCCCTTCCTTCTTTAACTT
GGTGTCTGAGGGGTTTTGTCTGTGGCTTGTCCTGCTACATTTCTTGGTT
CCCTGACCAGGAAGCAAAGTGATTAACGGACAGTTGAGGCAGCCCCTTA
GGCAGCTTAGGCCTGCCTTGTGGAGCATCCCCGCGGGGAACTCTGGCCA
GCTTGAGCGACACGGATCCTCAGAGCGCTCCCAGGTAGGCAATTGCCCC
AGTGGAATGCCTCGTCAGAGCAGTGCATGGCAGGCCCCTGTGGAGGATC
AACGCAGTGGCTGAACACAGGGAAGGAACTGGCACTTGGAGTCCGGACA
ACTGAAACTTGTCGCTTCCTGCCTCGGACGGCTCAGCTGGTATGACCCA
GATTTCCAGGCAAGGCTCACCCGTTCCAACTCGAAGTGCCAGGGCCAGC
TGGAGGTCTACCTCAAGGACGGATGGCACATGGTTTGCAGCCAGAGCTG
GGGCCGGAGCTCCAAGCAGTGGGAGGACCCCAGTCAAGCGTCAAAAGTC
TGCCAGCGGCTGAACTGTGGGGTGCCCTTAAGCCTTGGCCCCTTCCTTG
TCACCTACACACCTCAGAGCTCAATCATCTGCTACGGACAACTGGGCTC
CTTCTCCAACTGCAGCCACAGCAGAAATGACATGTGTCACTCTCTGGGC
CTGACCTGCTTAGAACCCCAGAAGACAACACCTCCAACGACAAGGCCCC
CGCCCACCACAACTCCAGAGCCCACAGCTCCTCCCAGGCTGCAGCTGGT
GGCACAGTCTGGCGGCCAGCACTGTGCCGGCGTGGTGGAGTTCTACAGC
GGCAGCCTGGGGGGTACCATCAGCTATGAGGCCCAGGACAAGACCCAGG
ACCTGGAGAACTTCCTCTGCAACAACCTCCAGTGTGGCTCCTTCTTGAA
GCATCTGCCAGAGACTGAGGCAGGCAGAGCCCAAGACCCAGGGGAGCCA
CGGGAACACCAGCCCTTGCCAATCCAATGGAAGATCCAGAACTCAAGCT
GTACCTCCCTGGAGCATTGCTTCAGGAAAATCAAGCCCCAGAAAAGTGG
CCGAGTTCTTGCCCTCCTTTGCTCAGGTTTCCAGCCCAAGGTGCAGAGC
CGTCTGGTGGGGGGCAGCAGCATCTGTGAAGGCACCGTGGAGGTGCGCC
AGGGGGCTCAGTGGGCAGCCCTGTGTGACAGCTCTTCAGCCAGGAGCTC
GCTGCGGTGGGAGGAGGTGTGCCGGGAGCAGCAGTGTGGCAGCGTCAAC
TCCTATCGAGTGCTGGACGCTGGTGACCCAACATCCCGGGGGCTCTTCT
GTCCCCATCAGAAGCTGTCCCAGTGCCACGAACTTTGGGAGAGAAATTC
CTACTGCAAGAAGGTGTTTGTCACATGCCAGGATCCAAACCCCGCAGGC
CTGGCCGCAGGCACGGTGGCAAGCATCATCCTGGCCCTGGTGCTCCTGG
TGGTGCTGCTGGTCGTGTGCGGCCCCCTTGCCTACAAGAAGCTAGTGAA
GAAATTCCGCCAGAAGAAGCAGCGCCAGTGGATTGGCCCAACGGGAATG
AACCAAAACATGTCTTTCCATCGCAACCACACGGCAACCGTCCGATCCC
ATGCTGAGAACCCCACAGCCTCCCACGTGGATAACGAATACAGCCAACC
TCCCAGGAACTCCCACCTGTCAGCTTATCCAGCTCTGGAAGGGGCTCTG
CATCGCTCCTCCATGCAGCCTGACAACTCCTCCGACAGTGACTATGATC
TGCATGGGGCTCAGAGGCTGTAAAGAACTGGGATCCATGAGCAAAAAGC
CGAGAGCCAGACCTGTTTGTCCTGAGAAAACTGTCCGCTCTTCACTTGA
AATCATGTCCCTATTTCTACCCCGGCCAGAACATGGACAGAGGCCAGAA
GCCTTCCGGACAGGCGCTGCTGCCCCGAGTGGCAGGCCAGCTCACACTC
TGCTGCACAACAGCTCGGCCGCCCCTCCACTTGTGGAAGCTGTGGTGGG
CAGAGCCCCAAAACAAGCAGCCTTCCAACTAGAGACTCGGGGGTGTCTG
AAGGGGGCCCCCTTTCCCTGCCCGCTGGGGAGCGGCGTCTCAGTGAAAT
CGGCTTTCTCCTCAGACTCTGTCCCTGGTAAGGAGTGACAAGGAAGCTC
ACAGCTGGGCGAGTGCATTTTGAATAGTTTTTTGTAAGTAGTGCTTTTC
CTCCTTCCTGACAAATCGAGCGCTTTGGCCTCTTCTGTGCAGCATCCAC
CCCTGCGGATCCCTCTGGGGAGGACAGGAAGGGGACTCCCGGAGACCTC
TGCAGCCGTGGTGGTCAGAGGCTGCTCACCTGAGCACAAAGACAGCTCT
GCACATTCACCGCAGCTGCCAGCCAGGGGTCTGGGTGGGCACCACCCTG
ACCCACAGCGTCACCCCACTCCCTCTGTCTTATGACTCCCCTCCCCAAC
CCCCTCATCTAAAGACACCTTCCTTTCCACTGGCTGTCAAGCCCACAGG
GCACCAGTGCCACCCAGGGCCCGGCACAAAGGGGCGCCTAGTAAACCTT
AACCAACTTGGTTTTTTGCTTCACCCAGCAATTAAAAGTCCCAAGCTGA
GGTAGTTTCAGTCCATCACAGTTCATCTTCTAACCCAAGAGTCAGAGAT
GGGGCTGGTCATGTTCCTTTGGTTTGAATAACTCCCTTGACGAAAACAG
ACTCCTCTAGTACTTGGAGATCTTGGACGTACACCTAATCCCATGGGGC
CTCGGCTTCCTTAACTGCAAGTGAGAAGAGGAGGTCTACCCAGGAGCCT
CGGGTCTGATCAAGGGAGAGGCCAGGCGCAGCTCACTGCGGCGGCTCCC
TAAGAAGGTGAAGCAACATGGGAACACATCCTAAGACAGGTCCTTTCTC
CACGCCATTTGATGCTGTATCTCCTGGGAGCACAGGCATCAATGGTCCA
AGCCGCATAATAAGTCTGGAAGAGCAAAAGGGAGTTACTAGGATATGGG
GTGGGCTGCTCCCAGAATCTGCTCAGCTTTCTGCCCCCACCAACACCCT
CCAACCAGGCCTTGCCTTCTGAGAGCCCCCGTGGCCAAGCCCAGGTCAC
AGATCTTCCCCCGACCATGCTGGGAATCCAGAAACAGGGACCCCATTTG
TCTTCCCATATCTGGTGGAGGTGAGGGGGCTCCTCAAAAGGGAACTGAG
AGGCTGCTCTTAGGGAGGGCAAAGGTTCGGGGGCAGCCAGTGTCTCCCA
TCAGTGCCTTTTTTAATAAAAGCTCTTTCATCTATAGTTTGGCCACCAT
ACAGTGGCCTCAAAGCAACCATGGCCTACTTAAAAACCAAACCAAAAAT
AAAGAGTTTAGTTGAGGAGAAAAAAAAAAAAAAAAAAAAAAAAA

By “cluster of differentiation 7 (CD7)” is meant a protein having at least about 85% amino acid sequence identity to NCBI Accession No. NP_006128.1 or fragment thereof and having immunomodulatory activity. An exemplary amino acid sequence is provided below.

>NP_006128.1 T-cell antigen CD7 precursor [Homo sapiens]

MAGPPRLLLLPLLLALARGLPGALAAQEVQQSPHCTTVPVGASVNITCS
TSGGLRGIYLRQLGPQPQDIIYYEDGVVPTTDRRFRGRIDFSGSQDNLT
ITMHRLQLSDTGTYTCQAITEVNVYGSGTLVLVTEEQSQGWHRCSDAPP
RASALPAPPTGSALPDPQTASALPDPPAASALPAALAVISFLLGLGLGV
ACVLARTQIKKLCSWRDKNSAACVVYEDMSHSRCNTLSSPNQYQ

By “cluster of differentiation 7 (CD7)” is meant a nucleic acid encoding a CD7 polypeptide. An exemplary CD7 nucleic acid sequence is provided below.

>NM_006137.7 Homo sapiens CD7 molecule (CD7), mRNA

CTCTCTGAGCTCTGAGCGCCTGCGGTCTCCTGTGTGCTGCTCTCTGTGG
GGTCCTGTAGACCCAGAGAGGCTCAGCTGCACTCGCCCGGCTGGGAGAG
CTGGGTGTGGGGAACATGGCCGGGCCTCCGAGGCTCCTGCTGCTGCCCC
TGCTTCTGGCGCTGGCTCGCGGCCTGCCTGGGGCCCTGGCTGCCCAAGA
GGTGCAGCAGTCTCCCCACTGCACGACTGTCCCCGTGGGAGCCTCCGTC
AACATCACCTGCTCCACCAGCGGGGGCCTGCGTGGGATCTACCTGAGGC
AGCTCGGGCCACAGCCCCAAGACATCATTTACTACGAGGACGGGGTGGT
GCCCACTACGGACAGACGGTTCCGGGGCCGCATCGACTTCTCAGGGTCC
CAGGACAACCTGACTATCACCATGCACCGCCTGCAGCTGTCGGACACTG
GCACCTACACCTGCCAGGCCATCACGGAGGTCAATGTCTACGGCTCCGG
CACCCTGGTCCTGGTGACAGAGGAACAGTCCCAAGGATGGCACAGATGC
TCGGACGCCCCACCAAGGGCCTCTGCCCTCCCTGCCCCACCGACAGGCT
CCGCCCTCCCTGACCCGCAGACAGCCTCTGCCCTCCCTGACCCGCCAGC
AGCCTCTGCCCTCCCTGCGGCCCTGGCGGTGATCTCCTTCCTCCTCGGG
CTGGGCCTGGGGGTGGCGTGTGTGCTGGCGAGGACACAGATAAAGAAAC
TGTGCTCGTGGCGGGATAAGAATTCGGCGGCATGTGTGGTGTACGAGGA
CATGTCGCACAGCCGCTGCAACACGCTGTCCTCCCCCAACCAGTACCAG
TGACCCAGTGGGCCCCTGCACGTCCCGCCTGTGGTCCCCCCAGCACCTT
CCCTGCCCCACCATGCCCCCCACCCTGCCACACCCCTCACCCTGCTGTC
CTCCCACGGCTGCAGCAGAGTTTGAAGGGCCCAGCCGTGCCCAGCTCCA
AGCAGACACACAGGCAGTGGCCAGGCCCCACGGTGCTTCTCAGTGGACA
ATGATGCCTCCTCCGGGAAGCCTTCCCTGCCCAGCCCACGCCGCCACCG
GGAGGAAGCCTGACTGTCCTTTGGCTGCATCTCCCGACCATGGCCAAGG
AGGGCTTTTCTGTGGGATGGGCCTGGGCACGCGGCCCTCTCCTGTCAGT
GCCGGCCCACCCACCAGCAGGCCCCCAACCCCCAGGCAGCCCGGCAGAG
GACGGGAGGAGACCAGTCCCCCACCCAGCCGTACCAGAAATAAAGGCTT
CTGTGCTTCC

By “cluster of differentiation 30 (CD30)” is meant a protein having at least about 85% amino acid sequence identity to NCBI Accession No. NP_001234.3 or fragment thereof and having immunomodulatory activity. An exemplary amino acid sequence is provided below.

>NP_001234.3 tumor necrosis factor receptor superfamily member 8 isoform 1 precursor [Homo sapiens]

MRVLLAALGLLFLGALRAFPQDRPFEDTCHGNPSHYYDKAVRRCCYRCPMG
LFPTQQCPQRPTDCRKQCEPDYYLDEADRCTACVTCSRDDLVEKTPCAWNS
SRVCECRPGMFCSTSAVNSCARCFFHSVCPAGMIVKFPGTAQKNTVCEPAS
PGVSPACASPENCKEPSSGTIPQAKPTPVSPATSSASTMPVRGGTRLAQEA
ASKLTRAPDSPSSVGRPSSDPGLSPTQPCPEGSGDCRKQCEPDYYLDEAGR
CTACVSCSRDDLVEKTPCAWNSSRTCECRPGMICATSATNSCARCVPYPIC
AAETVTKPQDMAEKDTTFEAPPLGTQPDCNPTPENGEAPASTSPTQSLLVD
SQASKTLPIPTSAPVALSSTGKPVLDAGPVLFWVILVLVVVVGSSAFLLCH
RRACRKRIRQKLHLCYPVQTSQPKLELVDSRPRRSSTQLRSGASVTEPVAE
ERGLMSQPLMETCHSVGAAYLESLPLQDASPAGGPSSPRDLPEPRVSTEHT
NNKIEKIYIMKADTVIVGTVKAELPEGRGLAGPAEPELEEELEADHTPHYP
EQETEPPLGSCSDVMLSVEEEGKEDPLPTAASGK

By “cluster of differentiation 30 (CD30)” is meant a nucleic acid encoding a CD30 polypeptide. An exemplary CD30 nucleic acid sequence is provided below. >NM_001243.5 Homo sapiens TNF receptor superfamily member 8 (TNFRSF8), transcript variant 1, mRNA

CTGAGTCATCTCTGCACGTGTTTGCCCCCTTTTTTCTTCGCTGCTTGTAGC
TAAGTGTTCCTGGAACCAATTTGATACGGGAGAACTAAGGCTGAAACCTCG
GAGGAACAACCACTTTTGAAGTGACTTCGCGGCGTGCGTTGGGTGCGGACT
AGGTGGCCGCGGCGGGAGTGTGCTGGAGCCTGAAGTCCACGCGCGCGGCTG
AGAACCGCCGGGACCGCACGTGGGCGCCGCGCGCTTCCCCCGCTTCCCAGG
TGGGCGCCGGCCGCCAGGCCACCTCACGTCCGGCCCCGGGGATGCGCGTCC
TCCTCGCCGCGCTGGGACTGCTGTTCCTGGGGGCGCTACGAGCCTTCCCAC
AGGATCGACCCTTCGAGGACACCTGTCATGGAAACCCCAGCCACTACTATG
ACAAGGCTGTCAGGAGGTGCTGTTACCGCTGCCCCATGGGGCTGTTCCCGA
CACAGCAGTGCCCACAGAGGCCTACTGACTGCAGGAAGCAGTGTGAGCCTG
ACTACTACCTGGATGAGGCCGACCGCTGTACAGCCTGCGTGACTTGTTCTC
GAGACGACCTCGTGGAGAAGACGCCGTGTGCATGGAACTCCTCCCGTGTCT
GCGAATGTCGACCCGGCATGTTCTGTTCCACGTCTGCCGTCAACTCCTGTG
CCCGCTGCTTCTTCCATTCTGTCTGTCCGGCAGGGATGATTGTCAAGTTCC
CAGGCACGGCGCAGAAGAACACGGTCTGTGAGCCGGCTTCCCCAGGGGTCA
GCCCTGCCTGTGCCAGCCCAGAGAACTGCAAGGAACCCTCCAGTGGCACCA
TCCCCCAGGCCAAGCCCACCCCGGTGTCCCCAGCAACCTCCAGTGCCAGCA
CCATGCCTGTAAGAGGGGGCACCCGCCTCGCCCAGGAAGCTGCTTCTAAAC
TGACGAGGGCTCCCGACTCTCCCTCCTCTGTGGGAAGGCCTAGTTCAGATC
CAGGTCTGTCCCCAACACAGCCATGCCCAGAGGGGTCTGGTGATTGCAGAA
AGCAGTGTGAGCCCGACTACTACCTGGACGAGGCCGGCCGCTGCACGGCCT
GCGTGAGCTGTTCTCGAGATGACCTTGTGGAGAAGACGCCATGTGCATGGA
ACTCCTCCCGCACCTGCGAATGTCGACCTGGCATGATCTGTGCCACATCAG
CCACCAACTCCTGTGCCCGCTGTGTCCCCTACCCAATCTGTGCAGCAGAGA
CGGTCACCAAGCCCCAGGATATGGCTGAGAAGGACACCACCTTTGAGGCGC
CACCCCTGGGGACCCAGCCGGACTGCAACCCCACCCCAGAGAATGGCGAGG
CGCCTGCCAGCACCAGCCCCACTCAGAGCTTGCTGGTGGACTCCCAGGCCA
GTAAGACGCTGCCCATCCCAACCAGCGCTCCCGTCGCTCTCTCCTCCACGG
GGAAGCCCGTTCTGGATGCAGGGCCAGTGCTCTTCTGGGTGATCCTGGTGT
TGGTTGTGGTGGTCGGCTCCAGCGCCTTCCTCCTGTGCCACCGGAGGGCCT
GCAGGAAGCGAATTCGGCAGAAGCTCCACCTGTGCTACCCGGTCCAGACCT
CCCAGCCCAAGCTAGAGCTTGTGGATTCCAGACCCAGGAGGAGCTCAACGC
AGCTGAGGAGTGGTGCGTCGGTGACAGAACCCGTCGCGGAAGAGCGAGGGT
TAATGAGCCAGCCACTGATGGAGACCTGCCACAGCGTGGGGGCAGCCTACC
TGGAGAGCCTGCCGCTGCAGGATGCCAGCCCGGCCGGGGGCCCCTCGTCCC
CCAGGGACCTTCCTGAGCCCCGGGTGTCCACGGAGCACACCAATAACAAGA
TTGAGAAAATCTACATCATGAAGGCTGACACCGTGATCGTGGGGACCGTGA
AGGCTGAGCTGCCGGAGGGCCGGGGCCTGGCGGGGCCAGCAGAGCCCGAGT
TGGAGGAGGAGCTGGAGGCGGACCATACCCCCCACTACCCCGAGCAGGAGA
CAGAACCGCCTCTGGGCAGCTGCAGCGATGTCATGCTCTCAGTGGAAGAGG
AAGGGAAAGAAGACCCCTTGCCCACAGCTGCCTCTGGAAAGTGAGGCCTGG
GCTGGGCTGGGGCTAGGAGGGCAGCAGGGTGGCCTCTGGGAGGCCAGGATG
GCACTGTTGGCACCGAGGTTGGGGGCAGAGGCCCATCTGGCCTGAACTGAG
GCTCCAGCATCTAGTGGTGGACCGGCCGGTCACTGCAGGGGTCTGGTGGTC
TCTGCTTGCATCCCCAACTTAGCTGTCCCCTGACCCAGAGCCTAGGGGATC
CGGGGCTTGTACAGAAGAGACAGTCCAAGGGGACTGGATCCCAGCAGTGAT
GTTGGTTGAGGCAGCAAACAGATGGCAGGATGGGCACTGCCGAGAACAGCA
TTGGTCCCAGAGCCCTGGGCATCAGACCTTAACCACCAGGCCCACAGCCCA
GCGAGGGAGAGGTCGTGAGGCCAGCTCCCGGGGCCCCTGTAACCCTACTCT
CCTCTCTCCCTGGACCTCAGAGGTGACACCCATTGGGCCCTTCCGGCATGC
CCCCAGTTACTGTAAATGTGGCCCCCAGTGGGCATGGAGCCAGTGCCTGTG
GTTGTTTCTCCAGAGTCAAAAGGGAAGTCGAGGGATGGGGCGTCGTCAGCT
GGCACTGTCTCTGCTGCAGCGGCCACACTGTACTCTGCACTGGTGTGAGGG
CCCCTGCCTGGACTGTGGGACCCTCCTGGTGCTGCCCACCTTCCCTGTCCT
GTAGCCCCCTCGGTGGGCCCAGGGCCTAGGGCCCAGGATCAAGTCACTCAT
CTCAGAATGTCCCCACCAATCCCCGCCACAGCAGGCGCCTCGGGTCCCAGA
TGTCTGCAGCCCTCAGCAGCTGCAGACCGCCCCTCACCAACCCAGAGAACC
TGCTTTACTTTGCCCAGGGACTTCCTCCCCATGTGAACATGGGGAACTTCG
GGCCCTGCCTGGAGTCCTTGACCGCTCTCTGTGGGCCCCACCCACTCTGTC
CTGGGAAATGAAGAAGCATCTTCCTTAGGTCTGCCCTGCTTGCAAATCCAC
TAGCACCGACCCCACCACCTGGTTCCGGCTCTGCACGCTTTGGGGTGTGGA
TGTCGAGAGGCACCACGGCCTCACCCAGGCATCTGCTTTACTCTGGACCAT
AGGAAACAAGACCGTTTGGAGGTTTCATCAGGATTTTGGGTTTTTCACATT
TCACGCTAAGGAGTAGTGGCCCTGACTTCCGGTCGGCTGGCCAGCTGACTC
CCTAGGGCCTTCAGACGTGTATGCAAATGAGTGATGGATAAGGATGAGTCT
TGGAGTTGCGGGCAGCCTGGAGACTCGTGGACTTACCGCCTGGAGGCAGGC
CCGGGAAGGCTGCTGTTTACTCATCGGGCAGCCACGTGCTCTCTGGAGGAA
GTGATAGTTTCTGAAACCGCTCAGATGTTTTGGGGAAAGTTGGAGAAGCCG
TGGCCTTGCGAGAGGTGGTTACACCAGAACCTGGACATTGGCCAGAAGAAG
CTTAAGTGGGCAGACACTGTTTGCCCAGTGTTTGTGCAAGGATGGAGTGGG
TGTCTCTGCATCACCCACAGCCGCAGCTGTAAGGCACGCTGGAAGGCACAC
GCCTGCCAGGCAGGGCAGTCTGGCGCCCATGATGGGAGGGATTGACATGTT
TCAACAAAATAATGCACTTCCTTACCTAGTGGCCCTTCACACAACTTTTGA
ATCTCTAAAAATCCATAAAATCCTTAAAGAACTGTAA

By “cluster of differentiation 33 (CD33)” is meant a protein having at least about 85% amino acid sequence identity to NCBI Accession No. NP_001763.3 or fragment thereof and having immunomodulatory activity. An exemplary amino acid sequence is provided below.

>NP 001763.3 myeloid cell surface antigen CD33 isoform 1 precursor [Homo sapiens]

MPLLLLLPLLWAGALAMDPNFWLQVQESVTVQEGLCVLVPCTFFHPIPYYD
KNSPVHGYWFREGAIISRDSPVATNKLDQEVQEETQGRFRLLGDPSRNNCS
LSIVDARRRDNGSYFFRMERGSTKYSYKSPQLSVHVTDLTHRPKILIPGTL
EPGHSKNLTCSVSWACEQGTPPIFSWLSAAPTSLGPRTTHSSVLIITPRPQ
DHGTNLTCQVKFAGAGVTTERTIQLNVTYVPQNPTTGIFPGDGSGKQETRA
GVVHGAIGGAGVTALLALCLCLIFFIVKTHRRKAARTAVGRNDTHPTTGSA
SPKHQKKSKLHGPTETSSCSGAAPTVEMDEELHYASLNFHGMNPSKDTSTE
YSEVRTQ

By “cluster of differentiation 33 (CD33)” is meant a nucleic acid encoding a CD33 polypeptide. An exemplary CD33 nucleic acid sequence is provided below. >NM_001772.4 Homo sapiens CD33 molecule (CD33), transcript variant 1, mRNA

CTGCTCACACAGGAAGCCCTGGAAGCTGCTTCCTCAGACATGCCGCTG
CTGCTACTGCTGCCCCTGCTGTGGGCAGGGGCCCTGGCTATGGATCCA
AATTTCTGGCTGCAAGTGCAGGAGTCAGTGACGGTACAGGAGGGTTTG
TGCGTCCTCGTGCCCTGCACTTTCTTCCATCCCATACCCTACTACGAC
AAGAACTCCCCAGTTCATGGTTACTGGTTCCGGGAAGGAGCCATTATA
TCCAGGGACTCTCCAGTGGCCACAAACAAGCTAGATCAAGAAGTACAG
GAGGAGACTCAGGGCAGATTCCGCCTCCTTGGGGATCCCAGTAGGAAC
AACTGCTCCCTGAGCATCGTAGACGCCAGGAGGAGGGATAATGGTTCA
TACTTCTTTCGGATGGAGAGAGGAAGTACCAAATACAGTTACAAATCT
CCCCAGCTCTCTGTGCATGTGACAGACTTGACCCACAGGCCCAAAATC
CTCATCCCTGGCACTCTAGAACCCGGCCACTCCAAAAACCTGACCTGC
TCTGTGTCCTGGGCCTGTGAGCAGGGAACACCCCCGATCTTCTCCTGG
TTGTCAGCTGCCCCCACCTCCCTGGGCCCCAGGACTACTCACTCCTCG
GTGCTCATAATCACCCCACGGCCCCAGGACCACGGCACCAACCTGACC
TGTCAGGTGAAGTTCGCTGGAGCTGGTGTGACTACGGAGAGAACCATC
CAGCTCAACGTCACCTATGTTCCACAGAACCCAACAACTGGTATCTTT
CCAGGAGATGGCTCAGGGAAACAAGAGACCAGAGCAGGAGTGGTTCAT
GGGGCCATTGGAGGAGCTGGTGTTACAGCCCTGCTCGCTCTTTGTCTC
TGCCTCATCTTCTTCATAGTGAAGACCCACAGGAGGAAAGCAGCCAGG
ACAGCAGTGGGCAGGAATGACACCCACCCTACCACAGGGTCAGCCTCC
CCGAAACACCAGAAGAAGTCCAAGTTACATGGCCCCACTGAAACCTCA
AGCTGTTCAGGTGCCGCCCCTACTGTGGAGATGGATGAGGAGCTGCAT
TATGCTTCCCTCAACTTTCATGGGATGAATCCTTCCAAGGACACCTCC
ACCGAATACTCAGAGGTCAGGACCCAGTGAGGAACCCACAAGAGCATC
AGGCTCAGCTAGAAGATCCACATCCTCTACAGGTCGGGGACCAAAGGC
TGATTCTTGGAGATTTAACACCCCACAGGCAATGGGTTTATAGACATT
ATGTGAGTTTCCTGCTATATTAACATCATCTTAGACTTTGCAAGCAGA
GAGTCGTGGAATCAAATCTGTGCTCTTTCATTTGCTAAGTGTATGATG
TCACACAAGCTCCTTAACCTTCCATGTCTCCATTTTCTTCTCTGTGAA
GTAGGTATAAGAAGTCCTATCTCATAGGGATGCTGTGAGCATTAAATA
AAGGTACACATGGAAAACACCA

By “cluster of differentiation 52 (CD52)” is meant a protein having at least about 85% amino acid sequence identity to NCBI Accession No. NP_001794.2 or fragment thereof and having immunomodulatory activity. An exemplary amino acid sequence is provided below.

>NP_001794.2 CAMPATH-1 antigen precursor [Homo sapiens]

MKRFLFLLLTISLLVMVQIQTGLSGQNDTSQTSSPSASSNISGGIFLFFVA
NAIIHLFCFS

By “cluster of differentiation 52 (CD52)” is meant a nucleic acid encoding a CD52 polypeptide. An exemplary CD52 nucleic acid sequence is provided below. >NM_001803.3 Homo sapiens CD52 molecule (CD52), mRNA

AGACAGCCCTGAGATCACCTAAAAAGCTGCTACCAAGACAGCCACGAA
GATCCTACCAAAATGAAGCGCTTCCTCTTCCTCCTACTCACCATCAGC
CTCCTGGTTATGGTACAGATACAAACTGGACTCTCAGGACAAAACGAC
ACCAGCCAAACCAGCAGCCCCTCAGCATCCAGCAACATAAGCGGAGGC
ATTTTCCTTTTCTTCGTGGCCAATGCCATAATCCACCTCTTCTGCTTC
AGTTGAGGTGACACGTCTCAGCCTTAGCCCTGTGCCCCCTGAAACAGC
TGCCACCATCACTCGCAAGAGAATCCCCTCCATCTTTGGGAGGGGTTG
ATGCCAGACATCACCAGGTTGTAGAAGTTGACAGGCAGTGCCATGGGG
GCAACAGCCAAAATAGGGGGGTAATGATGTAGGGGCCAAGCAGTGCCC
AGCTGGGGGTCAATAAAGTTACCCTTGTACTTGCA

By “cluster of differentiation 70 (CD70)” is meant a protein having at least about 85% amino acid sequence identity to NCBI Accession No. NP_001243.1 or fragment thereof and having immunomodulatory activity. An exemplary amino acid sequence is provided below.

>NP_001243.1 CD70 antigen isoform 1 [Homo sapiens]

MPEEGSGCSVRRRPYGCVLRAALVPLVAGLVICLVVCIQRFAQAQQQL
PLESLGWDVAELQLNHTGPQQDPRLYWQGGPALGRSFLHGPELDKGQL
RIHRDGIYMVHIQVTLAICSSTTASRHHPTTLAVGICSPASRSISLLR
LSFHQGCTIASQRLTPLARGDTLCTNLTGTLLPSRNTDETFFGVQWVRP

By “cluster of differentiation 70 (CD70)” is meant a nucleic acid encoding a CD70 polypeptide. An exemplary CD70 nucleic acid sequence is provided below. >NM_001252.5 Homo sapiens CD70 molecule (CD70), transcript variant 1, mRNA

AGAGAGGGGCAGGCTGGTCCCCTGACAGGTTGAAGCAAGTAGACGCC
CAGGAGCCCCGGGAGGGGGCTGCAGTTTCCTTCCTTCCTTCTCGGCA
GCGCTCCGCGCCCCCATCGCCCCTCCTGCGCTAGCGGAGGTGATCGC
CGCGGCGATGCCGGAGGAGGGTTCGGGCTGCTCGGTGCGGCGCAGGC
CCTATGGGTGCGTCCTGCGGGCTGCTTTGGTCCCATTGGTCGCGGGC
TTGGTGATCTGCCTCGTGGTGTGCATCCAGCGCTTCGCACAGGCTCA
GCAGCAGCTGCCGCTCGAGTCACTTGGGTGGGACGTAGCTGAGCTGC
AGCTGAATCACACAGGACCTCAGCAGGACCCCAGGCTATACTGGCAG
GGGGGCCCAGCACTGGGCCGCTCCTTCCTGCATGGACCAGAGCTGGA
CAAGGGGCAGCTACGTATCCATCGTGATGGCATCTACATGGTACACA
TCCAGGTGACGCTGGCCATCTGCTCCTCCACGACGGCCTCCAGGCAC
CACCCCACCACCCTGGCCGTGGGAATCTGCTCTCCCGCCTCCCGTAG
CATCAGCCTGCTGCGTCTCAGCTTCCACCAAGGTTGTACCATTGCCT
CCCAGCGCCTGACGCCCCTGGCCCGAGGGGACACACTCTGCACCAAC
CTCACTGGGACACTTTTGCCTTCCCGAAACACTGATGAGACCTTCTT
TGGAGTGCAGTGGGTGCGCCCCTGACCACTGCTGCTGATTAGGGTTT
TTTAAATTTTATTTTATTTTATTTAAGTTCAAGAGAAAAAGTGTACA
CACAGGGGCCACCCGGGGTTGGGGTGGGAGTGTGGTGGGGGGTAGTG
GTGGCAGGACAAGAGAAGGCATTGAGCTTTTTCTTTCATTTTCCTAT
TAAAAAATACAAAAATCA

By “class II, major histocompatibility complex, transactivator (CIITA)” is meant a protein having at least about 85% amino acid sequence identity to NCBI Accession No. NP_001273331.1 or fragment thereof and having immunomodulatory activity. An exemplary amino acid sequence is provided below.

>NP_001273331.1 MHC class II transactivator isoform 1 [Homo sapiens]

MRCLAPRPAGSYLSEPQGSSQCATMELGPLEGGYLELLNSDADPLCLYHFY
DQMDLAGEEEIELYSEPDTDTINCDQFSRLLCDMEGDEETREAYANIAELD
QYVFQDSQLEGLSKDIFIEHIGPDEVIGESMEMPAEVGQKSQKRPFPEELP
ADLKHWKPAEPPTVVTGSLLVGPVSDCSTLPCLPLPALFNQEPASGQMRLE
KTDQIPMPFSSSSLSCLNLPEGPIQFVPTISTLPHGLWQISEAGTGVSSIF
IYHGEVPQASQVPPPSGFTVHGLPTSPDRPGSTSPFAPSATDLPSMPEPAL
TSRANMTEHKTSPTQCPAAGEVSNKLPKWPEPVEQFYRSLQDTYGAEPAGP
DGILVEVDLVQARLERSSSKSLERELATPDWAERQLAQGGLAEVLLAAKEH
RRPRETRVIAVLGKAGQGKSYWAGAVSRAWACGRLPQYDFVFSVPCHCLNR
PGDAYGLQDLLFSLGPQPLVAADEVFSHILKRPDRVLLILDGFEELEAQDG
FLHSTCGPAPAEPCSLRGLLAGLFQKKLLRGCTLLLTARPRGRLVQSLSKA
DALFELSGFSMEQAQAYVMRYFESSGMTEHQDRALTLLRDRPLLLSHSHSP
TLCRAVCQLSEALLELGEDAKLPSTLTGLYVGLLGRAALDSPPGALAELAK
LAWELGRRHQSTLQEDQFPSADVRTWAMAKGLVQHPPRAAESELAFPSFLL
QCFLGALWLALSGEIKDKELPQYLALTPRKKRPYDNWLEGVPRFLAGLIFQ
PPARCLGALLGPSAAASVDRKQKVLARYLKRLQPGTLRARQLLELLHCAHE
AEEAGIWQHVVQELPGRLSFLGTRLTPPDAHVLGKALEAAGQDFSLDLRST
GICPSGLGSLVGLSCVTRFRAALSDTVALWESLQQHGETKLLQAAEEKFTI
EPFKAKSLKDVEDLGKLVQTQRTRSSSEDTAGELPAVRDLKKLEFALGPVS
GPQAFPKLVRILTAFSSLQHLDLDALSENKIGDEGVSQLSATFPQLKSLET
LNLSQNNITDLGAYKLAEALPSLAASLLRLSLYNNCICDVGAESLARVLPD
MVSLRVMDVQYNKFTAAGAQQLAASLRRCPHVETLAMWTPTIPFSVQEHLQ
QQDSRISLR

By “class II, major histocompatibility complex, transactivator (CIITA)” is meant a nucleic acid encoding a CIITA polypeptide. An exemplary CIITA nucleic acid sequence is provided below.

>NM_001286402.1 Homo sapiens class II major histocompatibility complex transactivator (CIITA), transcript variant 1, mRNA

GGTTAGTGATGAGGCTAGTGATGAGGCTGTGTGCTTCTGAGCTGGGCATCC
GAAGGCATCCTTGGGGAAGCTGAGGGCACGAGGAGGGGCTGCCAGACTCCG
GGAGCTGCTGCCTGGCTGGGATTCCTACACAATGCGTTGCCTGGCTCCACG
CCCTGCTGGGTCCTACCTGTCAGAGCCCCAAGGCAGCTCACAGTGTGCCAC
CATGGAGTTGGGGCCCCTAGAAGGTGGCTACCTGGAGCTTCTTAACAGCGA
TGCTGACCCCCTGTGCCTCTACCACTTCTATGACCAGATGGACCTGGCTGG
AGAAGAAGAGATTGAGCTCTACTCAGAACCCGACACAGACACCATCAACTG
CGACCAGTTCAGCAGGCTGTTGTGTGACATGGAAGGTGATGAAGAGACCAG
GGAGGCTTATGCCAATATCGCGGAACTGGACCAGTATGTCTTCCAGGACTC
CCAGCTGGAGGGCCTGAGCAAGGACATTTTCATAGAGCACATAGGACCAGA
TGAAGTGATCGGTGAGAGTATGGAGATGCCAGCAGAAGTTGGGCAGAAAAG
TCAGAAAAGACCCTTCCCAGAGGAGCTTCCGGCAGACCTGAAGCACTGGAA
GCCAGCTGAGCCCCCCACTGTGGTGACTGGCAGTCTCCTAGTGGGACCAGT
GAGCGACTGCTCCACCCTGCCCTGCCTGCCACTGCCTGCGCTGTTCAACCA
GGAGCCAGCCTCCGGCCAGATGCGCCTGGAGAAAACCGACCAGATTCCCAT
GCCTTTCTCCAGTTCCTCGTTGAGCTGCCTGAATCTCCCTGAGGGACCCAT
CCAGTTTGTCCCCACCATCTCCACTCTGCCCCATGGGCTCTGGCAAATCTC
TGAGGCTGGAACAGGGGTCTCCAGTATATTCATCTACCATGGTGAGGTGCC
CCAGGCCAGCCAAGTACCCCCTCCCAGTGGATTCACTGTCCACGGCCTCCC
AACATCTCCAGACCGGCCAGGCTCCACCAGCCCCTTCGCTCCATCAGCCAC
TGACCTGCCCAGCATGCCTGAACCTGCCCTGACCTCCCGAGCAAACATGAC
AGAGCACAAGACGTCCCCCACCCAATGCCCGGCAGCTGGAGAGGTCTCCAA
CAAGCTTCCAAAATGGCCTGAGCCGGTGGAGCAGTTCTACCGCTCACTGCA
GGACACGTATGGTGCCGAGCCCGCAGGCCCGGATGGCATCCTAGTGGAGGT
GGATCTGGTGCAGGCCAGGCTGGAGAGGAGCAGCAGCAAGAGCCTGGAGCG
GGAACTGGCCACCCCGGACTGGGCAGAACGGCAGCTGGCCCAAGGAGGCCT
GGCTGAGGTGCTGTTGGCTGCCAAGGAGCACCGGCGGCCGCGTGAGACACG
AGTGATTGCTGTGCTGGGCAAAGCTGGTCAGGGCAAGAGCTATTGGGCTGG
GGCAGTGAGCCGGGCCTGGGCTTGTGGCCGGCTTCCCCAGTACGACTTTGT
CTTCTCTGTCCCCTGCCATTGCTTGAACCGTCCGGGGGATGCCTATGGCCT
GCAGGATCTGCTCTTCTCCCTGGGCCCACAGCCACTCGTGGCGGCCGATGA
GGTTTTCAGCCACATCTTGAAGAGACCTGACCGCGTTCTGCTCATCCTAGA
CGGCTTCGAGGAGCTGGAAGCGCAAGATGGCTTCCTGCACAGCACGTGCGG
ACCGGCACCGGCGGAGCCCTGCTCCCTCCGGGGGCTGCTGGCCGGCCTTTT
CCAGAAGAAGCTGCTCCGAGGTTGCACCCTCCTCCTCACAGCCCGGCCCCG
GGGCCGCCTGGTCCAGAGCCTGAGCAAGGCCGACGCCCTATTTGAGCTGTC
CGGCTTCTCCATGGAGCAGGCCCAGGCATACGTGATGCGCTACTTTGAGAG
CTCAGGGATGACAGAGCACCAAGACAGAGCCCTGACGCTCCTCCGGGACCG
GCCACTTCTTCTCAGTCACAGCCACAGCCCTACTTTGTGCCGGGCAGTGTG
CCAGCTCTCAGAGGCCCTGCTGGAGCTTGGGGAGGACGCCAAGCTGCCCTC
CACGCTCACGGGACTCTATGTCGGCCTGCTGGGCCGTGCAGCCCTCGACAG
CCCCCCCGGGGCCCTGGCAGAGCTGGCCAAGCTGGCCTGGGAGCTGGGCCG
CAGACATCAAAGTACCCTACAGGAGGACCAGTTCCCATCCGCAGACGTGAG
GACCTGGGCGATGGCCAAAGGCTTAGTCCAACACCCACCGCGGGCCGCAGA
GTCCGAGCTGGCCTTCCCCAGCTTCCTCCTGCAATGCTTCCTGGGGGCCCT
GTGGCTGGCTCTGAGTGGCGAAATCAAGGACAAGGAGCTCCCGCAGTACCT
AGCATTGACCCCAAGGAAGAAGAGGCCCTATGACAACTGGCTGGAGGGCGT
GCCACGCTTTCTGGCTGGGCTGATCTTCCAGCCTCCCGCCCGCTGCCTGGG
AGCCCTACTCGGGCCATCGGCGGCTGCCTCGGTGGACAGGAAGCAGAAGGT
GCTTGCGAGGTACCTGAAGCGGCTGCAGCCGGGGACACTGCGGGCGCGGCA
GCTGCTGGAGCTGCTGCACTGCGCCCACGAGGCCGAGGAGGCTGGAATTTG
GCAGCACGTGGTACAGGAGCTCCCCGGCCGCCTCTCTTTTCTGGGCACCCG
CCTCACGCCTCCTGATGCACATGTACTGGGCAAGGCCTTGGAGGCGGCGGG
CCAAGACTTCTCCCTGGACCTCCGCAGCACTGGCATTTGCCCCTCTGGATT
GGGGAGCCTCGTGGGACTCAGCTGTGTCACCCGTTTCAGGGCTGCCTTGAG
CGACACGGTGGCGCTGTGGGAGTCCCTGCAGCAGCATGGGGAGACCAAGCT
ACTTCAGGCAGCAGAGGAGAAGTTCACCATCGAGCCTTTCAAAGCCAAGTC
CCTGAAGGATGTGGAAGACCTGGGAAAGCTTGTGCAGACTCAGAGGACGAG
AAGTTCCTCGGAAGACACAGCTGGGGAGCTCCCTGCTGTTCGGGACCTAAA
GAAACTGGAGTTTGCGCTGGGCCCTGTCTCAGGCCCCCAGGCTTTCCCCAA
ACTGGTGCGGATCCTCACGGCCTTTTCCTCCCTGCAGCATCTGGACCTGGA
TGCGCTGAGTGAGAACAAGATCGGGGACGAGGGTGTCTCGCAGCTCTCAGC
CACCTTCCCCCAGCTGAAGTCCTTGGAAACCCTCAATCTGTCCCAGAACAA
CATCACTGACCTGGGTGCCTACAAACTCGCCGAGGCCCTGCCTTCGCTCGC
TGCATCCCTGCTCAGGCTAAGCTTGTACAATAACTGCATCTGCGACGTGGG
AGCCGAGAGCTTGGCTCGTGTGCTTCCGGACATGGTGTCCCTCCGGGTGAT
GGACGTCCAGTACAACAAGTTCACGGCTGCCGGGGCCCAGCAGCTCGCTGC
CAGCCTTCGGAGGTGTCCTCATGTGGAGACGCTGGCGATGTGGACGCCCAC
CATCCCATTCAGTGTCCAGGAACACCTGCAACAACAGGATTCACGGATCAG
CCTGAGATGATCCCAGCTGTGCTCTGGACAGGCATGTTCTCTGAGGACACT
AACCACGCTGGACCTTGAACTGGGTACTTGTGGACACAGCTCTTCTCCAGG
CTGTATCCCATGAGCCTCAGCATCCTGGCACCCGGCCCCTGCTGGTTCAGG
GTTGGCCCCTGCCCGGCTGCGGAATGAACCACATCTTGCTCTGCTGACAGA
CACAGGCCCGGCTCCAGGCTCCTTTAGCGCCCAGTTGGGTGGATGCCTGGT
GGCAGCTGCGGTCCACCCAGGAGCCCCGAGGCCTTCTCTGAAGGACATTGC
GGACAGCCACGGCCAGGCCAGAGGGAGTGACAGAGGCAGCCCCATTCTGCC
TGCCCAGGCCCCTGCCACCCTGGGGAGAAAGTACTTCTTTTTTTTTATTTT
TAGACAGAGTCTCACTGTTGCCCAGGCTGGCGTGCAGTGGTGCGATCTGGG
TTCACTGCAACCTCCGCCTCTTGGGTTCAAGCGATTCTTCTGCTTCAGCCT
CCCGAGTAGCTGGGACTACAGGCACCCACCATCATGTCTGGCTAATTTTTC
ATTTTTAGTAGAGACAGGGTTTTGCCATGTTGGCCAGGCTGGTCTCAAACT
CTTGACCTCAGGTGATCCACCCACCTCAGCCTCCCAAAGTGCTGGGATTAC
AAGCGTGAGCCACTGCACCGGGCCACAGAGAAAGTACTTCTCCACCCTGCT
CTCCGACCAGACACCTTGACAGGGCACACCGGGCACTCAGAAGACACTGAT
GGGCAACCCCCAGCCTGCTAATTCCCCAGATTGCAACAGGCTGGGCTTCAG
TGGCAGCTGCTTTTGTCTATGGGACTCAATGCACTGACATTGTTGGCCAAA
GCCAAAGCTAGGCCTGGCCAGATGCACCAGCCCTTAGCAGGGAAACAGCTA
ATGGGACACTAATGGGGCGGTGAGAGGGGAACAGACTGGAAGCACAGCTTC
ATTTCCTGTGTCTTTTTTCACTACATTATAAATGTCTCTTTAATGTCACAG
GCAGGTCCAGGGTTTGAGTTCATACCCTGTTACCATTTTGGGGTACCCACT
GCTCTGGTTATCTAATATGTAACAAGCCACCCCAAATCATAGTGGCTTAAA
ACAACACTCACATTTA

By “cytotoxic T-lymphocyte associated protein 4 (CTLA-4) polypeptide” is meant a protein having at least about 85% sequence identity to NCBI Accession No. EAW70354.1 or a fragment thereof. An exemplary amino acid sequence is provided below:

>EAW70354.1 cytotoxic T-lymphocyte-associated protein 4 [Homo sapiens]

MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN

By “cytotoxic T-lymphocyte associated protein 4 (CTLA-4) polynucleotide” is meant a nucleic acid molecule encoding a CTLA-4 polypeptide. The CTLA-4 gene encodes an immunoglobulin superfamily and encodes a protein which transmits an inhibitory signal to T cells. An exemplary CTLA-4 nucleic acid sequence is provided below.

>BC074842.2 Homo sapiens cytotoxic T-lymphocyte-associated protein 4, mRNA (cDNA clone MGC:104099 IMAGE:30915552), complete cds

GACCTGAACACCGCTCCCATAAAGCCATGGCTTGCCTTGGATTTCAGCGGC
ACAAGGCTCAGCTGAACCTGGCTACCAGGACCTGGCCCTGCACTCTCCTGT
TTTTTCTTCTCTTCATCCCTGTCTTCTGCAAAGCAATGCACGTGGCCCAGC
CTGCTGTGGTACTGGCCAGCAGCCGAGGCATCGCCAGCTTTGTGTGTGAGT
ATGCATCTCCAGGCAAAGCCACTGAGGTCCGGGTGACAGTGCTTCGGCAGG
CTGACAGCCAGGTGACTGAAGTCTGTGCGGCAACCTACATGATGGGGAATG
AGTTGACCTTCCTAGATGATTCCATCTGCACGGGCACCTCCAGTGGAAATC
AAGTGAACCTCACTATCCAAGGACTGAGGGCCATGGACACGGGACTCTACA
TCTGCAAGGTGGAGCTCATGTACCCACCGCCATACTACCTGGGCATAGGCA
ACGGAACCCAGATTTATGTAATTGATCCAGAACCGTGCCCAGATTCTGACT
TCCTCCTCTGGATCCTTGCAGCAGTTAGTTCGGGGTTGTTTTTTTATAGCT
TTCTCCTCACAGCTGTTTCTTTGAGCAAAATGCTAAAGAAAAGAAGCCCTC
TTACAACAGGGGTCTATGTGAAAATGCCCCCAACAGAGCCAGAATGTGAAA
AGCAATTTCAGCCTTATTTTATTCCCATCAATTGAGAAACCATTATGAAGA
AGAGAGTCCATATTTCAATTTCCAAGAGCTGAGG

By “cytidine deaminase” is meant a polypeptide or fragment thereof capable of catalyzing a deamination reaction that converts an amino group to a carbonyl group. In one embodiment, the cytidine deaminase converts cytosine to uracil or 5-methylcytosine to thymine. PmCDA1 derived from Petromyzon marinus (Petromyzon marinus cytosine deaminase 1), or AID (Activation-induced cytidine deaminase; AICDA) derived from mammal (e.g., human, swine, bovine, horse, monkey etc.), and APOBEC are exemplary cytidine deaminases.

The base sequence and amino acid sequence of PmCDA1 and the base sequence and amino acid sequence of human AID are shown below.

>tr|A5H718|A5H718_PETMA Cytosine deaminase OS=Petromyzon marinus OX=7757 PE=2 SV=1

MTDAEYVRIHEKLDIYTFKKQFFNNKKSVSHRCYVLFELKRRGERRACFW
GYAVNKPQSGTERGIHAEIFSIRKVEEYLRDNPGQFTINWYSSWSPCADC
AEKILEWYNQELRGNGHTLKIWACKLYYEKNARNQIGLWNLRDNGVGLNV
MVSEHYQCCRKIFIQSSHNQLNENRWLEKTLKRAEKRRSELSIMIQVKIL
HTTKSPAV

>EF094822.1 Petromyzon marinus isolate PmCDA.21 cytosine deaminase mRNA, complete cds

TGACACGACACAGCCGTGTATATGAGGAAGGGTAGCTGGATGGGGGGGGGG
GGAATACGTTCAGAGAGGACATTAGCGAGCGTCTTGTTGGTGGCCTTGAGT
CTAGACACCTGCAGACATGACCGACGCTGAGTACGTGAGAATCCATGAGAA
GTTGGACATCTACACGTTTAAGAAACAGTTTTTCAACAACAAAAAATCCGT
GTCGCATAGATGCTACGTTCTCTTTGAATTAAAACGACGGGGTGAACGTAG
AGCGTGTTTTTGGGGCTATGCTGTGAATAAACCACAGAGCGGGACAGAACG
TGGAATTCACGCCGAAATCTTTAGCATTAGAAAAGTCGAAGAATACCTGCG
CGACAACCCCGGACAATTCACGATAAATTGGTACTCATCCTGGAGTCCTTG
TGCAGATTGCGCTGAAAAGATCTTAGAATGGTATAACCAGGAGCTGCGGGG
GAACGGCCACACTTTGAAAATCTGGGCTTGCAAACTCTATTACGAGAAAAA
TGCGAGGAATCAAATTGGGCTGTGGAACCTCAGAGATAACGGGGTTGGGTT
GAATGTAATGGTAAGTGAACACTACCAATGTTGCAGGAAAATATTCATCCA
ATCGTCGCACAATCAATTGAATGAGAATAGATGGCTTGAGAAGACTTTGAA
GCGAGCTGAAAAACGACGGAGCGAGTTGTCCATTATGATTCAGGTAAAAAT
ACTCCACACCACTAAGAGTCCTGCTGTTTAAGAGGCTATGCGGATGGTTTT
C

>tr|Q6QJ80|Q6QJ80 HUMAN Activation-induced cytidine deaminase OS═Homo sapiens OX=9606 GN=AICDA PE=2 SV=1

MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKAPV

>NG_011588.1:5001-15681 Homo sapiens activation induced cytidine deaminase (AICDA), RefSeqGene (LRG_17) on chromosome 12

AGAGAACCATCATTAATTGAAGTGAGATTTTTCTGGCCTGAGACTTGCAG
GGAGGCAAGAAGACACTCTGGACACCACTATGGACAGGTAAAGAGGCAGT
CTTCTCGTGGGTGATTGCACTGGCCTTCCTCTCAGAGCAAATCTGAGTAA
TGAGACTGGTAGCTATCCCTTTCTCTCATGTAACTGTCTGACTGATAAGA
TCAGCTTGATCAATATGCATATATATTTTTTGATCTGTCTCCTTTTCTTC
TATTCAGATCTTATACGCTGTCAGCCCAATTCTTTCTGTTTCAGACTTCT
CTTGATTTCCCTCTTTTTCATGTGGCAAAAGAAGTAGTGCGTACAATGTA
CTGATTCGTCCTGAGATTTGTACCATGGTTGAAACTAATTTATGGTAATA
ATATTAACATAGCAAATCTTTAGAGACTCAAATCATGAAAAGGTAATAGC
AGTACTGTACTAAAAACGGTAGTGCTAATTTTCGTAATAATTTTGTAAAT
ATTCAACAGTAAAACAACTTGAAGACACACTTTCCTAGGGAGGCGTTACT
GAAATAATTTAGCTATAGTAAGAAAATTTGTAATTTTAGAAATGCCAAGC
ATTCTAAATTAATTGCTTGAAAGTCACTATGATTGTGTCCATTATAAGGA
GACAAATTCATTCAAGCAAGTTATTTAATGTTAAAGGCCCAATTGTTAGG
CAGTTAATGGCACTTTTACTATTAACTAATCTTTCCATTTGTTCAGACGT
AGCTTAACTTACCTCTTAGGTGTGAATTTGGTTAAGGTCCTCATAATGTC
TTTATGTGCAGTTTTTGATAGGTTATTGTCATAGAACTTATTCTATTCCT
ACATTTATGATTACTATGGATGTATGAGAATAACACCTAATCCTTATACT
TTACCTCAATTTAACTCCTTTATAAAGAACTTACATTACAGAATAAAGAT
TTTTTAAAAATATATTTTTTTGTAGAGACAGGGTCTTAGCCCAGCCGAGG
CTGGTCTCTAAGTCCTGGCCCAAGCGATCCTCCTGCCTGGGCCTCCTAAA
GTGCTGGAATTATAGACATGAGCCATCACATCCAATATACAGAATAAAGA
TTTTTAATGGAGGATTTAATGTTCTTCAGAAAATTTTCTTGAGGTCAGAC
AATGTCAAATGTCTCCTCAGTTTACACTGAGATTTTGAAAACAAGTCTGA
GCTATAGGTCCTTGTGAAGGGTCCATTGGAAATACTTGTTCAAAGTAAAA
TGGAAAGCAAAGGTAAAATCAGCAGTTGAAATTCAGAGAAAGACAGAAAA
GGAGAAAAGATGAAATTCAACAGGACAGAAGGGAAATATATTATCATTAA
GGAGGACAGTATCTGTAGAGCTCATTAGTGATGGCAAAATGACTTGGTCA
GGATTATTTTTAACCCGCTTGTTTCTGGTTTGCACGGCTGGGGATGCAGC
TAGGGTTCTGCCTCAGGGAGCACAGCTGTCCAGAGCAGCTGTCAGCCTGC
AAGCCTGAAACACTCCCTCGGTAAAGTCCTTCCTACTCAGGACAGAAATG
ACGAGAACAGGGAGCTGGAAACAGGCCCCTAACCAGAGAAGGGAAGTAAT
GGATCAACAAAGTTAACTAGCAGGTCAGGATCACGCAATTCATTTCACTC
TGACTGGTAACATGTGACAGAAACAGTGTAGGCTTATTGTATTTTCATGT
AGAGTAGGACCCAAAAATCCACCCAAAGTCCTTTATCTATGCCACATCCT
TCTTATCTATACTTCCAGGACACTTTTTCTTCCTTATGATAAGGCTCTCT
CTCTCTCCACACACACACACACACACACACACACACACACACACACACAC
ACAAACACACACCCCGCCAACCAAGGTGCATGTAAAAAGATGTAGATTCC
TCTGCCTTTCTCATCTACACAGCCCAGGAGGGTAAGTTAATATAAGAGGG
ATTTATTGGTAAGAGATGATGCTTAATCTGTTTAACACTGGGCCTCAAAG
AGAGAATTTCTTTTCTTCTGTACTTATTAAGCACCTATTATGTGTTGAGC
TTATATATACAAAGGGTTATTATATGCTAATATAGTAATAGTAATGGTGG
TTGGTACTATGGTAATTACCATAAAAATTATTATCCTTTTAAAATAAAGC
TAATTATTATTGGATCTTTTTTAGTATTCATTTTATGTTTTTTATGTTTT
TGATTTTTTAAAAGACAATCTCACCCTGTTACCCAGGCTGGAGTGCAGTG
GTGCAATCATAGCTTTCTGCAGTCTTGAACTCCTGGGCTCAAGCAATCCT
CCTGCCTTGGCCTCCCAAAGTGTTGGGATACAGTCATGAGCCACTGCATC
TGGCCTAGGATCCATTTAGATTAAAATATGCATTTTAAATTTTAAAATAA
TATGGCTAATTTTTACCTTATGTAATGTGTATACTGGCAATAAATCTAGT
TTGCTGCCTAAAGTTTAAAGTGCTTTCCAGTAAGCTTCATGTACGTGAGG
GGAGACATTTAAAGTGAAACAGACAGCCAGGTGTGGTGGCTCACGCCTGT
AATCCCAGCACTCTGGGAGGCTGAGGTGGGTGGATCGCTTGAGCCCTGGA
GTTCAAGACCAGCCTGAGCAACATGGCAAAACGCTGTTTCTATAACAAAA
ATTAGCCGGGCATGGTGGCATGTGCCTGTGGTCCCAGCTACTAGGGGGCT
GAGGCAGGAGAATCGTTGGAGCCCAGGAGGTCAAGGCTGCACTGAGCAGT
GCTTGCGCCACTGCACTCCAGCCTGGGTGACAGGACCAGACCTTGCCTCA
AAAAAATAAGAAGAAAAATTAAAAATAAATGGAAACAACTACAAAGAGCT
GTTGTCCTAGATGAGCTACTTAGTTAGGCTGATATTTTGGTATTTAACTT
TTAAAGTCAGGGTCTGTCACCTGCACTACATTATTAAAATATCAATTCTC
AATGTATATCCACACAAAGACTGGTACGTGAATGTTCATAGTACCTTTAT
TCACAAAACCCCAAAGTAGAGACTATCCAAATATCCATCAACAAGTGAAC
AAATAAACAAAATGTGCTATATCCATGCAATGGAATACCACCCTGCAGTA
CAAAGAAGCTACTTGGGGATGAATCCCAAAGTCATGACGCTAAATGAAAG
AGTCAGACATGAAGGAGGAGATAATGTATGCCATACGAAATTCTAGAAAA
TGAAAGTAACTTATAGTTACAGAAAGCAAATCAGGGCAGGCATAGAGGCT
CACACCTGTAATCCCAGCACTTTGAGAGGCCACGTGGGAAGATTGCTAGA
ACTCAGGAGTTCAAGACCAGCCTGGGCAACACAGTGAAACTCCATTCTCC
ACAAAAATGGGAAAAAAAGAAAGCAAATCAGTGGTTGTCCTGTGGGGAGG
GGAAGGACTGCAAAGAGGGAAGAAGCTCTGGTGGGGTGAGGGTGGTGATT
CAGGTTCTGTATCCTGACTGTGGTAGCAGTTTGGGGTGTTTACATCCAAA
AATATTCGTAGAATTATGCATCTTAAATGGGTGGAGTTTACTGTATGTAA
ATTATACCTCAATGTAAGAAAAAATAATGTGTAAGAAAACTTTCAATTCT
CTTGCCAGCAAACGTTATTCAAATTCCTGAGCCCTTTACTTCGCAAATTC
TCTGCACTTCTGCCCCGTACCATTAGGTGACAGCACTAGCTCCACAAATT
GGATAAATGCATTTCTGGAAAAGACTAGGGACAAAATCCAGGCATCACTT
GTGCTTTCATATCAACCATGCTGTACAGCTTGTGTTGCTGTCTGCAGCTG
CAATGGGGACTCTTGATTTCTTTAAGGAAACTTGGGTTACCAGAGTATTT
CCACAAATGCTATTCAAATTAGTGCTTATGATATGCAAGACACTGTGCTA
GGAGCCAGAAAACAAAGAGGAGGAGAAATCAGTCATTATGTGGGAACAAC
ATAGCAAGATATTTAGATCATTTTGACTAGTTAAAAAAGCAGCAGAGTAC
AAAATCACACATGCAATCAGTATAATCCAAATCATGTAAATATGTGCCTG
TAGAAAGACTAGAGGAATAAACACAAGAATCTTAACAGTCATTGTCATTA
GACACTAAGTCTAATTATTATTATTAGACACTATGATATTTGAGATTTAA
AAAATCTTTAATATTTTAAAATTTAGAGCTCTTCTATTTTTCCATAGTAT
TCAAGTTTGACAATGATCAAGTATTACTCTTTCTTTTTTTTTTTTTTTTT
TTTTTTTTGAGATGGAGTTTTGGTCTTGTTGCCCATGCTGGAGTGGAATG
GCATGACCATAGCTCACTGCAACCTCCACCTCCTGGGTTCAAGCAAAGCT
GTCGCCTCAGCCTCCCGGGTAGATGGGATTACAGGCGCCCACCACCACAC
TCGGCTAATGTTTGTATTTTTAGTAGAGATGGGGTTTCACCATGTTGGCC
AGGCTGGTCTCAAACTCCTGACCTCAGAGGATCCACCTGCCTCAGCCTCC
CAAAGTGCTGGGATTACAGATGTAGGCCACTGCGCCCGGCCAAGTATTGC
TCTTATACATTAAAAAACAGGTGTGAGCCACTGCGCCCAGCCAGGTATTG
CTCTTATACATTAAAAAATAGGCCGGTGCAGTGGCTCACGCCTGTAATCC
CAGCACTTTGGGAAGCCAAGGCGGGCAGAACACCCGAGGTCAGGAGTCCA
AGGCCAGCCTGGCCAAGATGGTGAAACCCCGTCTCTATTAAAAATACAAA
CATTACCTGGGCATGATGGTGGGCGCCTGTAATCCCAGCTACTCAGGAGG
CTGAGGCAGGAGGATCCGCGGAGCCTGGCAGATCTGCCTGAGCCTGGGAG
GTTGAGGCTACAGTAAGCCAAGATCATGCCAGTATACTTCAGCCTGGGCG
ACAAAGTGAGACCGTAACAAAAAAAAAAAAATTTAAAAAAAGAAATTTAG
ATCAAGATCCAACTGTAAAAAGTGGCCTAAACACCACATTAAAGAGTTTG
GAGTTTATTCTGCAGGCAGAAGAGAACCATCAGGGGGTCTTCAGCATGGG
AATGGCATGGTGCACCTGGTTTTTGTGAGATCATGGTGGTGACAGTGTGG
GGAATGTTATTTTGGAGGGACTGGAGGCAGACAGACCGGTTAAAAGGCCA
GCACAACAGATAAGGAGGAAGAAGATGAGGGCTTGGACCGAAGCAGAGAA
GAGCAAACAGGGAAGGTACAAATTCAAGAAATATTGGGGGGTTTGAATCA
ACACATTTAGATGATTAATTAAATATGAGGACTGAGGAATAAGAAATGAG
TCAAGGATGGTTCCAGGCTGCTAGGCTGCTTACCTGAGGTGGCAAAGTCG
GGAGGAGTGGCAGTTTAGGACAGGGGGCAGTTGAGGAATATTGTTTTGAT
CATTTTGAGTTTGAGGTACAAGTTGGACACTTAGGTAAAGACTGGAGGGG
AAATCTGAATATACAATTATGGGACTGAGGAACAAGTTTATTTTATTTTT
TGTTTCGTTTTCTTGTTGAAGAACAAATTTAATTGTAATCCCAAGTCATC
AGCATCTAGAAGACAGTGGCAGGAGGTGACTGTCTTGTGGGTAAGGGTTT
GGGGTCCTTGATGAGTATCTCTCAATTGGCCTTAAATATAAGCAGGAAAA
GGAGTTTATGATGGATTCCAGGCTCAGCAGGGCTCAGGAGGGCTCAGGCA
GCCAGCAGAGGAAGTCAGAGCATCTTCTTTGGTTTAGCCCAAGTAATGAC
TTCCTTAAAAAGCTGAAGGAAAATCCAGAGTGACCAGATTATAAACTGTA
CTCTTGCATTTTCTCTCCCTCCTCTCACCCACAGCCTCTTGATGAACCGG
AGGAAGTTTCTTTACCAATTCAAAAATGTCCGCTGGGCTAAGGGTCGGCG
TGAGACCTACCTGTGCTACGTAGTGAAGAGGCGTGACAGTGCTACATCCT
TTTCACTGGACTTTGGTTATCTTCGCAATAAGGTATCAATTAAAGTCGGC
TTTGCAAGCAGTTTAATGGTCAACTGTGAGTGCTTTTAGAGCCACCTGCT
GATGGTATTACTTCCATCCTTTTTTGGCATTTGTGTCTCTATCACATTCC
TCAAATCCTTTTTTTTATTTCTTTTTCCATGTCCATGCACCCATATTAGA
CATGGCCCAAAATATGTGATTTAATTCCTCCCCAGTAATGCTGGGCACCC
TAATACCACTCCTTCCTTCAGTGCCAAGAACAACTGCTCCCAAACTGTTT
ACCAGCTTTCCTCAGCATCTGAATTGCCTTTGAGATTAATTAAGCTAAAA
GCATTTTTATATGGGAGAATATTATCAGCTTGTCCAAGCAAAAATTTTAA
ATGTGAAAAACAAATTGTGTCTTAAGCATTTTTGAAAATTAAGGAAGAAG
AATTTGGGAAAAAATTAACGGTGGCTCAATTCTGTCTTCCAAATGATTTC
TTTTCCCTCCTACTCACATGGGTCGTAGGCCAGTGAATACATTCAACATG
GTGATCCCCAGAAAACTCAGAGAAGCCTCGGCTGATGATTAATTAAATTG
ATCTTTCGGCTACCCGAGAGAATTACATTTCCAAGAGACTTCTTCACCAA
AATCCAGATGGGTTTACATAAACTTCTGCCCACGGGTATCTCCTCTCTCC
TAACACGCTGTGACGTCTGGGCTTGGTGGAATCTCAGGGAAGCATCCGTG
GGGTGGAAGGTCATCGTCTGGCTCGTTGTTTGATGGTTATATTACCATGC
AATTTTCTTTGCCTACATTTGTATTGAATACATCCCAATCTCCTTCCTAT
TCGGTGACATGACACATTCTATTTCAGAAGGCTTTGATTTTATCAAGCAC
TTTCATTTACTTCTCATGGCAGTGCCTATTACTTCTCTTACAATACCCAT
CTGTCTGCTTTACCAAAATCTATTTCCCCTTTTCAGATCCTCCCAAATGG
TCCTCATAAACTGTCCTGCCTCCACCTAGTGGTCCAGGTATATTTCCACA
ATGTTACATCAACAGGCACTTCTAGCCATTTTCCTTCTCAAAAGGTGCAA
AAAGCAACTTCATAAACACAAATTAAATCTTCGGTGAGGTAGTGTGATGC
TGCTTCCTCCCAACTCAGCGCACTTCGTCTTCCTCATTCCACAAAAACCC
ATAGCCTTCCTTCACTCTGCAGGACTAGTGCTGCCAAGGGTTCAGCTCTA
CCTACTGGTGTGCTCTTTTGAGCAAGTTGCTTAGCCTCTCTGTAACACAA
GGACAATAGCTGCAAGCATCCCCAAAGATCATTGCAGGAGACAATGACTA
AGGCTACCAGAGCCGCAATAAAAGTCAGTGAATTTTAGCGTGGTCCTCTC
TGTCTCTCCAGAACGGCTGCCACGTGGAATTGCTCTTCCTCCGCTACATC
TCGGACTGGGACCTAGACCCTGGCCGCTGCTACCGCGTCACCTGGTTCAC
CTCCTGGAGCCCCTGCTACGACTGTGCCCGACATGTGGCCGACTTTCTGC
GAGGGAACCCCAACCTCAGTCTGAGGATCTTCACCGCGCGCCTCTACTTC
TGTGAGGACCGCAAGGCTGAGCCCGAGGGGCTGCGGCGGCTGCACCGCGC
CGGGGTGCAAATAGCCATCATGACCTTCAAAGGTGCGAAAGGGCCTTCCG
CGCAGGCGCAGTGCAGCAGCCCGCATTCGGGATTGCGATGCGGAATGAAT
GAGTTAGTGGGGAAGCTCGAGGGGAAGAAGTGGGCGGGGATTCTGGTTCA
CCTCTGGAGCCGAAATTAAAGATTAGAAGCAGAGAAAAGAGTGAATGGCT
CAGAGACAAGGCCCCGAGGAAATGAGAAAATGGGGCCAGGGTTGCTTCTT
TCCCCTCGATTTGGAACCTGAACTGTCTTCTACCCCCATATCCCCGCCTT
TTTTTCCTTTTTTTTTTTTTGAAGATTATTTTTACTGCTGGAATACTTTT
GTAGAAAACCACGAAAGAACTTTCAAAGCCTGGGAAGGGCTGCATGAAAA
TTCAGTTCGTCTCTCCAGACAGCTTCGGCGCATCCTTTTGGTAAGGGGCT
TCCTCGCTTTTTAAATTTTCTTTCTTTCTCTACAGTCTTTTTTGGAGTTT
CGTATATTTCTTATATTTTCTTATTGTTCAATCACTCTCAGTTTTCATCT
GATGAAAACTTTATTTCTCCTCCACATCAGCTTTTTCTTCTGCTGTTTCA
CCATTCAGAGCCCTCTGCTAAGGTTCCTTTTCCCTCCCTTTTCTTTCTTT
TGTTGTTTCACATCTTTAAATTTCTGTCTCTCCCCAGGGTTGCGTTTCCT
TCCTGGTCAGAATTCTTTTCTCCTTTTTTTTTTTTTTTTTTTTTTTTTTT
AAACAAACAAACAAAAAACCCAAAAAAACTCTTTCCCAATTTACTTTCTT
CCAACATGTTACAAAGCCATCCACTCAGTTTAGAAGACTCTCCGGCCCCA
CCGACCCCCAACCTCGTTTTGAAGCCATTCACTCAATTTGCTTCTCTCTT
TCTCTACAGCCCCTGTATGAGGTTGATGACTTACGAGACGCATTTCGTAC
TTTGGGACTTTGATAGCAACTTCCAGGAATGTCACACACGATGAAATATC
TCTGCTGAAGACAGTGGATAAAAAACAGTCCTTCAAGTCTTCTCTGTTTT
TATTCTTCAACTCTCACTTTCTTAGAGTTTACAGAAAAAATATTTATATA
CGACTCTTTAAAAAGATCTATGTCTTGAAAATAGAGAAGGAACACAGGTC
TGGCCAGGGACGTGCTGCAATTGGTGCAGTTTTGAATGCAACATTGTCCC
CTACTGGGAATAACAGAACTGCAGGACCTGGGAGCATCCTAAAGTGTCAA
CGTTTTTCTATGACTTTTAGGTAGGATGAGAGCAGAAGGTAGATCCTAAA
AAGCATGGTGAGAGGATCAAATGTTTTTATATCAACATCCTTTATTATTT
GATTCATTTGAGTTAACAGTGGTGTTAGTGATAGATTTTTCTATTCTTTT
CCCTTGACGTTTACTTTCAAGTAACACAAACTCTTCCATCAGGCCATGAT
CTATAGGACCTCCTAATGAGAGTATCTGGGTGATTGTGACCCCAAACCAT
CTCTCCAAAGCATTAATATCCAATCATGCGCTGTATGTTTTAATCAGCAG
AAGCATGTTTTTATGTTTGTACAAAAGAAGATTGTTATGGGTGGGGATGG
AGGTATAGACCATGCATGGTCACCTTCAAGCTACTTTAATAAAGGATCTT
AAAATGGGCAGGAGGACTGTGAACAAGACACCCTAATAATGGGTTGATGT
CTGAAGTAGCAAATCTTCTGGAAACGCAAACTCTTTTAAGGAAGTCCCTA
ATTTAGAAACACCCACAAACTTCACATATCATAATTAGCAAACAATTGGA
AGGAAGTTGCTTGAATGTTGGGGAGAGGAAAATCTATTGGCTCTCGTGGG
TCTCTTCATCTCAGAAATGCCAATCAGGTCAAGGTTTGCTACATTTTGTA
TGTGTGTGATGCTTCTCCCAAAGGTATATTAACTATATAAGAGAGTTGTG
ACAAAACAGAATGATAAAGCTGCGAACCGTGGCACACGCTCATAGTTCTA
GCTGCTTGGGAGGTTGAGGAGGGAGGATGGCTTGAACACAGGTGTTCAAG
GCCAGCCTGGGCAACATAACAAGATCCTGTCTCTCAAAAAAAAAAAAAAA
AAAAAGAAAGAGAGAGGGCCGGGCGTGGTGGCTCACGCCTGTAATCCCAG
CACTTTGGGAGGCCGAGCCGGGCGGATCACCTGTGGTCAGGAGTTTGAGA
CCAGCCTGGCCAACATGGCAAAACCCCGTCTGTACTCAAAATGCAAAAAT
TAGCCAGGCGTGGTAGCAGGCACCTGTAATCCCAGCTACTTGGGAGGCTG
AGGCAGGAGAATCGCTTGAACCCAGGAGGTGGAGGTTGCAGTAAGCTGAG
ATCGTGCCGTTGCACTCCAGCCTGGGCGACAAGAGCAAGACTCTGTCTCA
GAAAAAAAAAAAAAAAAGAGAGAGAGAGAGAAAGAGAACAATATTTGGGA
GAGAAGGATGGGGAAGCATTGCAAGGAAATTGTGCTTTATCCAACAAAAT
GTAAGGAGCCAATAAGGGATCCCTATTTGTCTCTTTTGGTGTCTATTTGT
CCCTAACAACTGTCTTTGACAGTGAGAAAAATATTCAGAATAACCATATC
CCTGTGCCGTTATTACCTAGCAACCCTTGCAATGAAGATGAGCAGATCCA
CAGGAAAACTTGAATGCACAACTGTCTTATTTTAATCTTATTGTACATAA
GTTTGTAAAAGAGTTAAAAATTGTTACTTCATGTATTCATTTATATTTTA
TATTATTTTGCGTCTAATGATTTTTTATTAACATGATTTCCTTTTCTGAT
ATATTGAAATGGAGTCTCAAAGCTTCATAAATTTATAACTTTAGAAATGA
TTCTAATAACAACGTATGTAATTGTAACATTGCAGTAATGGTGCTACGAA
GCCATTTCTCTTGATTTTTAGTAAACTTTTATGACAGCAAATTTGCTTCT
GGCTCACTTTCAATCAGTTAAATAAATGATAAATAATTTTGGAAGCTGTG
AAGATAAAATACCAAATAAAATAATATAAAAGTGATTTATATGAAGTTAA
AATAAAAAATCAGTATGATGGAATAAACTTG

Apolipoprotein B mRNA editing enzyme, catalytic polypeptide-like (APOBEC) is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase. Many modified cytidine deaminases are commercially available, including but not limited to SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177).

Other exemplary deaminases that can be fused to Cas9 according to aspects of this disclosure are provided below. It should be understood that, in some embodiments, the active domain of the respective sequence can be used, e.g., the domain without a localizing signal (nuclear localization sequence, without nuclear export signal, cytoplasmic localizing signal).

Human AID:
MDSLLMNRRKFLYQFKNVRWAKGRRETYLCYVVKRRDSATSFSLDFGYLRNKNGCHV
ELLFLRYISDWDLDPGRCYRVTWFTSWSPCYDCARHVADFLRGNPNLSLRIFTARLYFC
EDRKAEPEGLRRLHRAGVQIAIMTFKDYFYCWNTFVENHERTFKAWEGLHENSVRLSR
QLRRILLPLYEVDDLRDAFRTLGL
(underline: nuclear localization sequence; double underline:
nuclear export signal)
Mouse AID:
MDSLLMKQKKFLYHFKNVRWAKGRHETYLCYVVKRRDSATSCSLDFGHLRNKSGCHV
ELLFLRYISDWDLDPGRCYRVTWFTSWSPCYDCARHVAEFLRWNPNLSLRIFTARLYFC
EDRKAEPEGLRRLHRAGVQIGIMTFKDYFYCWNTFVENRERTFKAWEGLHENSVRLTR
QLRRILLPLYEVDDLRDAFRMLGF
(underline: nuclear localization sequence; double underline:
nuclear export signal)
Canine AID:
MDSLLMKQRKFLYHFKNVRWAKGRHETYLCYVVKRRDSATSFSLDFGHLRNKSGCHV
ELLFLRYISDWDLDPGRCYRVTWFTSWSPCYDCARHVADFLRGYPNLSLRIFAARLYFC
EDRKAEPEGLRRLHRAGVQIAIMTFKDYFYCWNTFVENREKTFKAWEGLHENSVRLSR
QLRRILLPLYEVDDLRDAFRTLGL
(underline: nuclear localization sequence; double underline:
nuclear export signal)
Bovine AID:
MDSLLKKQRQFLYQFKNVRWAKGRHETYLCYVVKRRDSPTSFSLDFGHLRNKAGCHV
ELLFLRYISDWDLDPGRCYRVTWFTSWSPCYDCARHVADFLRGYPNLSLRIFTARLYFC
DKERKAEPEGLRRLHRAGVQIAIMTFKDYFYCWNTFVENHERTFKAWEGLHENSVRLS
RQLRRILLPLYEVDDLRDAFRTLGL
(underline: nuclear localization sequence; double underline:
nuclear export signal)
Rat AID
MAVGSKPKAALVGPHWERERIWCFLCSTGLGTQQTGQTSRWLRPAATQDPVSPPRSLL
MKQRKFLYHFKNVRWAKGRHETYLCYVVKRRDSATSFSLDFGYLRNKSGCHVELLFL
RYISDWDLDPGRCYRVTWFTSWSPCYDCARHVADFLRGNPNLSLRIFTARLTGWGALP
AGLMSPARPSDYFYCWNTFVENHERTFKAWEGLHENSVRLSRRLRRILLPLYEVDDLR
DAFRTLGL
(underline: nuclear localization sequence; double underline:
nuclear export signal)
Mouse APOBEC-3
MGPFCLGCSHRKCYSPIRNLISQETFKFHFKNLGYAKGRKDTFLCYEVTRKDCDSPVSLH
HGVFKNKDNIHAEICFLYWFHDKVLKVLSPREEFKITWYMSWSPCFECAEQIVRFLATHHN
LSLDIFSSRLYNVQDPETQQNLCRLVQEGAQVAAMDLYEFKKCWKKFVDNGGRRFRP
WKRLLTNFRYQDSKLQEILRPCYIPVPSSSS STLSNICLTKGLPETRFCVEGRRMDPLSEE
EFYSQFYNQRVKHLCYYHRMKPYLCYQLEQFNGQAPLKGCLLSEKGKQHAEILFLDKIR
SMELSQVTITCYLTWSPCPNCAWQLAAFKRDRPDLILHIYTSRLYFHWKRPFQKGLCSLW
QSGILVDVMDLPQFTDCWTNFVNPKRPFWPWKGLEIISRRTQRRLRRIKESWGLQDLVN
DFGNLQLGPPMS
(italic: nucleic acid editing domain)
Rat APOBEC-3:
MGPFCLGCSHRKCYSPIRNLISQETFKFHFKNRLRYAIDRKDTFLCYEVTRKDCDSPVSL
HHGVFKNKDNIHAEICFLYWFHDKVLKVLSPREEFKITWYMSWSPCFECAEQVLRFLATHH
NLSLDIFSSRLYNIRDPENQQNLCRLVQEGAQVAAMDLYEFKKCWKKFVDNGGRRFRP
WKKLLTNFRYQDSKLQEILRPCYIPVPSSSSSTLSNICLTKGLPETRFCVERRRVHLLSEEE
FYSQFYNQRVKHLCYYHGVKPYLCYQLEQFNGQAPLKGCLLSEKGKQHAEILFLDKIRS
MELSQVIITCYLTWSPCPNCAWQLAAFKRDRPDLILHIYTSRLYFHWKRPFQKGLCSLWQ
SGILVDVMDLPQFTDCWTNFVNPKRPFWPWKGLEIISRRTQRRLHRIKESWGLQDLVND
FGNLQLGPPMS
(italic: nucleic acid editing domain)
Rhesus macaque APOBEC-3 G:
MVEPMDPRTFVSNFNNRPILSGLNTVWLCCEVKTKDPSGPPLDAKIFQGKVYSKAKYHP
EMRFLRWFHKWRQLHHDQEYKVTWYVSWSPCTRCANSVATFLAKDPKVTLTIFVARL
YYFWKPDYQQALRILCQKRGGPHATMKIMNYNEFQDCWNKFVDGRGKPFKPRNNLPK
HYTLLQATLGELLRHLMDPGTFTSNFNNKPWVSGQHETYLCYKVERLHNDTWVPLNQ
HRGFLRNQAPNIHGFPKGRHAELCFLDLIPFWKLDGQQYRVTCFTSWSPCFSCAQEMAK
FISNNEHVSLCIFAARIYDDQGRYQEGLRALHRDGAKIAMMNYSEFEYCWDTFVDRQG
RPFQPWDGLDEHSQALSGRLRAI
(italic: nucleic acid editing domain; underline: cytoplasmic
localization signal)
Chimpanzee APOBEC-3 G:
MKPHFRNPVERMYQDTFSDNFYNRPILSHRNTVWLCYEVKTKGPSRPPLDAKIFRGQVY
SKLKYHPEMRFFHWFSKWRKLHRDQEYEVTWYISWSPCTKCTRDVATFLAEDPKVTLTIF
VARLYYFWDPDYQEALRSLCQKRDGPRATMKIMNYDEFQHCWSKFVYSQRELFEPWN
NLPKYYILLHIMLGEILRHSMDPPTFTSNFNNELWVRGRHETYLCYEVERLHNDTWVLL
NQRRGFLCNQAPHKHGFLEGRHAELCFLDVIPFWKLDLHQDYRVTCFTSWSPCFSCAQEM
AKFISNNKHVSLCIFAARIYDDQGRCQEGLRTLAKAGAKISIMTYSEFKHCWDTFVDHQ
GCPFQPWDGLEEHSQALSGRLRAILQNQGN
(italic: nucleic acid editing domain; underline: cytoplasmic
localization signal)
Green monkey APOBEC-3G:
MNPQIRNMVEQMEPDIFVYYFNNRPILSGRNTVWLCYEVKTKDPSGPPLDANIFQGKLY
PEAKDHPEMKFLHWFRKWRQLHRDQEYEVTWYVSWSPCTRCANSVATFLAEDPKVTLTIF
VARLYYFWKPDYQQALRILCQERGGPHATMKIMNYNEFQHCWNEFVDGQGKPFKPRK
NLPKHYTLLHATLGELLRHVMDPGTFTSNFNNKPWVSGQRETYLCYKVERSHNDTWV
LLNQHRGFLRNQAPDRHGFPKGRHAELCFLDLIPFWKLDDQQYRVTCFTSWSPCFSCAQK
MAKFISNNKHVSLCIFAARIYDDQGRCQEGLRTLHRDGAKIAVMNYSEFEYCWDTFVD
RQGRPFQPWDGLDEHSQALSGRLRAI
(italic: nucleic acid editing domain; underline: cytoplasmic
localization signal)
Human APOBEC-3G:
MKPHFRNTVERMYRDTFSYNFYNRPILSRRNTVWLCYEVKTKGPSRPPLDAKIFRGQVY
ESELKYHPEMRFFHWFSKWRKLHRDQEYEVTWYISWSPCTKCTRDMATFLAEDPKVTLTIF
VARLYYFWDPDYQEALRSLCQKRDGPRATMKIMNYDEFQHCWSKFVYSQRELFEPWN
NLPKYYILLHIMLGEILRHSMDPPTFTFNFNNEPWVRGRHETYLCYEVERMHNDTWVLL
NQRRGFLCNQAPHKHGFLEGRHAELCFLDVIPFWKLDLDQDYRVTCFTSWSPCFSCAQEM
AKFISKNKHVSLCIFTARIYDDQGRCQEGLRTLAEAGAKISIMTYSEFKHCWDTFVDHQ
GCPFQPWDGLDEHSQDLSGRLRAILQNQEN
(italic: nucleic acid editing domain; underline: cytoplasmic
localization signal)
Human APOBEC-3F:
MKPHFRNTVERMYRDTFSYNFYNRPILSRRNTVWLCYEVKTKGPSRPRLDAKIFRGQV
YSQPEHHAEMCFLSWFCGNQLPAYKCFQITWFVSWTPCPDCVAKLAEFLAEHPNVTLTIS
AARLYYYWERDYRRALCRLSQAGARVKIMDDEEFAYCWENFVYSEGQPFMPWYKFD
DNYAFLHRTLKEILRNPMEAMYPHIFYFHFKNLRKAYGRNESWLCFTMEVVKHHSPVS
WKRGVFRNQVDPETHCHAERCFLSWFCDDILSPNTNYEVTWYTSWSPCPECAGEVAEFLA
RHSNVNLTIFTARLYYFWDTDYQEGLRSLSQEGASVEIMGYKDFKYCWENFVYNDDEP
FKPWKGLKYNFLFLDSKLQEILE
(italic: nucleic acid editing domain)
Human APOBEC-3B:
MNPQIRNPMERMYRDTFYDNFENEPILYGRSYTWLCYEVKIKRGRSNLLWDTGVFRGQ
VYFKPQYHAEMCFLSWFCGNQLPAYKCFQITWFVSWTPCPDCVAKLAEFLSEHPNVTLTI
SAARLYYYWERDYRRALCRLSQAGARVTIMDYEEFAYCWENFVYNEGQQFMPWYKF
DENYAFLHRTLKEILRYLMDPDTFTFNFNNDPLVLRRRQTYLCYEVERLDNGTWVLMD
QHMGFLCNEAKNLLCGFYGRHAELRFLDLVPSLQLDPAQIYRVTWFISWSPCFSWGCAGE
VRAFLQENTHVRLRIFAARIYDYDPLYKEALQMLRDAGAQVSIMTYDEFEYCWDTFVY
RQGCPFQPWDGLEEHSQALSGRLRAILQNQGN
(italic: nucleic acid editing domain)
Rat APOBEC-3B:
MQPQGLGPNAGMGPVCLGCSHRRPYSPIRNPLKKLYQQTFYFHFKNVRYAWGRKNNF
LCYEVNGMDCALPVPLRQGVFRKQGHIHAELCFIYWFHDKVLRVLSPMEEFKVTWYM
SWSPCSKCAEQVARFLAAHRNLSLAIFSSRLYYYLRNPNYQQKLCRLIQEGVHVAAMD
LPEFKKCWNKFVDNDGQPFRPWMRLRINFSFYDCKLQEIFSRMNLLREDVFYLQFNNSH
RVKPVQNRYYRRKSYLCYQLERANGQEPLKGYLLYKKGEQHVEILFLEKMRSMELSQV
RITCYLTWSPCPNCARQLAAFKKDHPDLILRIYTSRLYFWRKKFQKGLCTLWRSGIHVD
VMDLPQFADCWTNFVNPQRPFRPWNELEKNSWRIQRRLRRIKESWGL
Bovine APOBEC-3B:
DGWEVAFRSGTVLKAGVLGVSMTEGWAGSGHPGQGACVWTPGTRNTMNLLREVLFK
QQFGNQPRVPAPYYRRKTYLCYQLKQRNDLTLDRGCFRNKKQRHAERFIDKINSLDLNP
SQSYKIICYITWSPCPNCANELVNFITRNNHLKLEIFASRLYFHWIKSFKMGLQDLQNAGI
SVAVMTHTEFEDCWEQFVDNQSRPFQPWDKLEQYSASIRRRLQRILTAPI
Chimpanzee APOBEC-3B:
MNPQIRNPMEWMYQRTFYYNFENEPILYGRSYTWLCYEVKIRRGHSNLLWDTGVFRGQ
MYSQPEHHAEMCFLSWFCGNQLSAYKCFQITWFVSWTPCPDCVAKLAKFLAEHPNVTL
TISAARLYYYWERDYRRALCRLSQAGARVKIMDDEEFAYCWENFVYNEGQPFMPWYK
FDDNYAFLHRTLKEIIRHLMDPDTFTFNFNNDPLVLRRHQTYLCYEVERLDNGTWVLM
DQHMGFLCNEAKNLLCGFYGRHAELRFLDLVPSLQLDPAQIYRVTWFISWSPCFSWGC
AGQVRAFLQENTHVRLRIFAARIYDYDPLYKEALQMLRDAGAQVSIMTYDEFEYCWDT
FVYRQGCPFQPWDGLEEHSQALSGRLRAILQVRASSLCMVPHRPPPPPQSPGPCLPLCSE
PPLGSLLPTGRPAPSLPFLLTASFSFPPPASLPPLPSLSLSPGHLPVPSFHSLTSCSIQPPCSSR
IRETEGWASVSKEGRDLG
Human APOBEC-3C:
MNPQIRNPMKAMYPGTFYFQFKNLWEANDRNETWLCFTVEGIKRRSVVSWKTGVFRN
QVDSETHCHAERCFLSWFCDDILSPNTKYQVTWYTSWSPCPDCAGEVAEFLARHSNVNLTI
FTARLYYFQYPCYQEGLRSLSQEGVAVEIMDYEDFKYCWENFVYNDNEPFKPWKGLKT
NFRLLKRRLRESLQ
(italic: nucleic acid editing domain)
Gorilla APOBEC3C
MNPQIRNPMKAMYPGTFYFQFKNLWEANDRNETWLCFTVEGIKRRSVVSWKTGVFRN
QVDSETHCHAERCFLSWECDDILSPNTNYQVTWYTSWSPCPECAGEVAEFLARHSNVNLTI
FTARLYYFQDTDYQEGLRSLSQEGVAVKIMDYKDFKYCWENFVYNDDEPFKPWKGLK
YNFRFLKRRLQEILE
(italic: nucleic acid editing domain)
Human APOBEC-3A:
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQENTH
VRLRIFAARIYDYDPLYKEALQMLRDAGAQVSIMTYDEFKHCWDTFVDHQGCPFQPWD
GLDEHSQALSGRLRAILQNQGN
(italic: nucleic acid editing domain)
Rhesus macaque APOBEC-3A:
MDGSPASRPRHLMDPNTFTFNFNNDLSVRGRHQTYLCYEVERLDNGTWVPMDERRGF
LCNKAKNVPCGDYGCHVELRFLCEVPSWQLDPAQTYRVTWFISWSPCFRRGCAGQVRVFL
QENKHVRLRIFAARIYDYDPLYQEALRTLRDAGAQVSIMTYEEFKHCWDTFVDRQGRP
FQPWDGLDEHSQALSGRLRAILQNQGN
(italic: nucleic acid editing domain)
Bovine APOBEC-3A:
MDEYTFTENFNNQGWPSKTYLCYEMERLDGDATIPLDEYKGFVRNKGLDQPEKPCHAE
LYFLGKIHSWNLDRNQHYRLTCFISWSPCYDCAQKLTTFLKENHHISLHILASRIYTHNRFG
CHQSGLCELQAAGARITIMTFEDFKHCWETFVDHKGKPFQPWEGLNVKSQALCTELQAI
LKTQQN
(italic: nucleic acid editing domain)
Human APOBEC-3H:
MALLTAETFRLQFNNKRRLRRPYYPRKALLCYQLTPQNGSTPTRGYFENKKKCHAEICFI
NEIKSMGLDETQCYQVTCYLTWSPCSSCAWELVDFIKAHDHLNLGIFASRLYYHWCKPQQ
KGLRLLCGSQVPVEVMGFPKFADCWENFVDHEKPLSFNPYKMLEELDKNSRAIKRRLE
RIKIPGVRAQGRYMDILCDAEV
(italic: nucleic acid editing domain)
Rhesus macaque APOBEC-3H:
MALLTAKTFSLQFNNKRRVNKPYYPRKALLCYQLTPQNGSTPTRGHLKNKKKDHAEIR
FINKIKSMGLDETQCYQVTCYLTWSPCPSCAGELVDFIKAHRHLNLRIFASRLYYHWRP
NYQEGLLLLCGSQVPVEVMGLPEFTDCWENFVDHKEPPSFNPSEKLEELDKNSQAIKRR
LERIKSRSVDVLENGLRSLQLGPVTPSSSIRNSR
Human APOBEC-3D:
MNPQIRNPMERMYRDTFYDNFENEPILYGRSYTWLCYEVKIKRGRSNLLWDTGVFRGP
VLPKRQSNHRQEVYFRFENHAEMCFLSWFCGNRLPANRRFQITWFVSWNPCLPCVVKVT
KFLAEHPNVTLTISAARLYYYRDRDWRWVLLRLHKAGARVKIMDYEDFAYCWENFVC
NEGQPFMPWYKFDDNYASLHRTLKEILRNPMEAMYPHIFYFHFKNLLKACGRNESWLC
FTMEVTKHHSAVFRKRGVFRNQVDPETHCHAERCFLSWPCDDILSPNTNYEVTWYTSWSP
CPECAGEVAEFLARHSNVNLTIFTARLCYFWDTDYQEGLCSLSQEGASVKIMGYKDFVS
CWKNFVYSDDEPFKPWKGLQTNFRLLKRRLREILQ
(italic: nucleic acid editing domain)
Human APOBEC-1:
MTSEKGPSTGDPTLRRRIEPWEFDVFYDPRELRKEACLLYEIKWGMSRKIWRSSGKNTT
NHVEVNFIKKFTSERDFHPSMSCSITWFLSWSPCWECSQAIREFLSRHPGVTLVIYVARLF
WHMDQQNRQGLRDLVNSGVTIQIMRASEYYHCWRNFVNYPPGDEAHWPQYPPLWMM
LYALELHCIILSLPPCLKISRRWQNHLTFFRLHLQNCHYQTIPPHILLATGLIHPSVAWR
Mouse APOBEC-1:
MSSETGPVAVDPTLRRRIEPHEFEVFFDPRELRKETCLLYEINWGGRHSVWRHTSQNTSN
HVEVNFLEKFTTERYFRPNTRCSITWFLSWSPCGECSRAITEFLSRHPYVTLFIYIARLYH
HTDQRNRQGLRDLISSGVTIQIMTEQEYCYCWRNFVNYPPSNEAYWPRYPHLWVKLYV
LELYCIILGLPPCLKILRRKQPQLTFFTITLQTCHYQRIPPHLLWATGLK
Rat APOBEC-1:
MSSETGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPHVTLFIYIARLYHH
ADPRNRQGLRDLISSGVTIQIMTEQESGYCWRNFVNYSPSNEAHWPRYPHLWVRLYVL
ELYCIILGLPPCLNILRRKQPQLTFFTIALQSCHYQRLPPHILWATGLK
Human APOBEC-2:
MAQKEEAAVATEAASQNGEDLENLDDPEKLKELIELPPFEIVTGERLPANFFKFQFRNVE
YSSGRNKTFLCYVVEAQGKGGQVQASRGYLEDEHAAAHAEEAFFNTILPAFDPALRYN
VTWYVSSSPCAACADRIIKTLSKTKNLRLLILVGRLFMWEEPEIQAALKKLKEAGCKLRI
MKPQDFEYVWQNFVEQEEGESKAFQPWEDIQENFLYYEEKLADILK
Mouse APOBEC-2:
MAQKEEAAEAAAPASQNGDDLENLEDPEKLKELIDLPPFEIVTGVRLPVNFFKFQFRNV
EYSSGRNKTFLCYVVEVQSKGGQAQATQGYLEDEHAGAHAEEAFFNTILPAFDPALKY
NVTWYVSSSPCAACADRILKTLSKTKNLRLLILVSRLFMWEEPEVQAALKKLKEAGCKL
RIMKPQDFEYIWQNFVEQEEGESKAFEPWEDIQENFLYYEEKLADILK
Rat APOBEC-2:
MAQKEEAAEAAAPASQNGDDLENLEDPEKLKELIDLPPFEIVTGVRLPVNFFKFQFRNV
EYSSGRNKTFLCYVVEAQSKGGQVQATQGYLEDEHAGAHAEEAFFNTILPAFDPALKY
NVTWYVSSSPCAACADRILKTLSKTKNLRLLILVSRLFMWEEPEVQAALKKLKEAGCKL
RIMKPQDFEYLWQNFVEQEEGESKAFEPWEDIQENFLYYEEKLADILK
Bovine APOBEC-2:
MAQKEEAAAAAEPASQNGEEVENLEDPEKLKELIELPPFEIVTGERLPAHYFKFQFRNVE
YSSGRNKTFLCYVVEAQSKGGQVQASRGYLEDEHATNHAEEAFFNSIMPTFDPALRYM
VTWYVSSSPCAACADRIVKTLNKTKNLRLLILVGRLFMWEEPEIQAALRKLKEAGCRLR
IMKPQDFEYIWQNFVEQEEGESKAFEPWEDIQENFLYYEEKLADILK
Petromyzon marinus CDA1 (pmCDA1)
MTDAEYVRIHEKLDIYTFKKQFFNNKKSVSHRCYVLFELKRRGERRACFWGYAVNKPQ
SGTERGIHAEIFSIRKVEEYLRDNPGQFTINWYSSWSPCADCAEKILEWYNQELRGNGHT
LKIWACKLYYEKNARNQIGLWNLRDNGVGLNVMVSEHYQCCRKIFIQSSHNQLNENR
WLEKTLKRAEKRRSELSFMIQVKILHTTKSPAV
Human APOBEC3G D316R D317R
MKPHFRNTVERMYRDTFSYNFYNRPILSRRNTVWLCYEVKTKGPSRPPLDAKIFRGQVY
SELKYHPEMRFFHWFSKWRKLHRDQEYEVTWYISWSPCTKCTRDMATFLAEDPKVTLT
IFVARLYYFWDPDYQEALRSLCQKRDGPRATMKFNYDEFQHCWSKFVYSQRELFEPWN
NLPKYYILLHFMLGEILRHSMDPPTFTFNFNNEPWVRGRHETYLCYEVERMHNDTWVL
LNQRRGFLCNQAPHKHGFLEGRHAELCFLDVIPFWKLDLDQDYRVTC
FTSWSPCFSCAQEMAKFISKKHVSLCIFTARIYRRQGRCQEGLRTLAEAGAKISFTYSEFK
HCWDTFVDHQGCPFQPWDGLDEHSQDLSGRLRAILQNQEN
Human APOBEC3G chain A
MDPPTFTFNFNNEPWWGRHETYLCYEVERMHNDTWVLLNQRRGFLCNQAPHKHGFLE
GRHAELCFLDVIPFWKLDLDQDYRVTCFTSWSPCFSCAQEMAKFISKNKHVSLCIFTARI
YDDQGRCQEGLRTLAEAGAKISFTYSEFKHCWDTFVDHQGCPFQPWDGLD
EHSQDLSGRLRAILQ
Human APOBEC3G chain A D120R D121R
MDPPTFTFNFNNEPWVRGRHETYLCYEVERMHNDTWVLLNQRRGFLCNQAPHKHGFL
EGRHAELCFLDVIPFWKLDLDQDYRVTCFTSWSPCFSCAQEMAKFISKNKHVSLCIFTAR
IYRRQGRCQEGLRTLAEAGAKISFMTYSEFKHCWDTFVDHQGCPFQPWDGLDEHSQDL
SGRLRAILQ

The term “deaminase” or “deaminase domain” refers to a protein or fragment thereof that catalyzes a deamination reaction. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the deaminase is a cytosine deaminase or an adenosine deaminase.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. In one embodiment, the disease is a neoplasia or cancer (e.g., multiple myeloma).

The term “effective amount,” as used herein, refers to an amount of a biologically active agent that is sufficient to elicit a desired biological response. In some embodiments, an effective amount of a fusion protein provided herein, e.g., of a cytidine deaminase or an adenosine deaminase nucleobase editor comprising a nCas9 domain and one or more deaminase domains (e.g., cytidine deaminase, adenosine deaminase) may refer to the amount of the fusion protein that is sufficient to induce editing of a target site specifically bound and edited by the cytidine deaminase or adenosine deaminase nucleobase editors. As will be appreciated by the skilled artisan, the effective amount of an agent, e.g., a fusion protein, may vary depending on various factors as, for example, on the desired biological response, e.g., on the specific allele, genome, or target site to be edited, on the cell or tissue being targeted, and on the agent being used. In the context of a CAR-T cell, “an effective amount refers” to the quantity of cells necessary to administer to a patient to achieve a therapeutic response.

In some embodiments, an effective amount of a fusion protein provided herein, e.g., of a fusion protein comprising a nCas9 domain and a cytidine deaminase or adenosine deaminase may refer to the amount of the fusion protein that is sufficient to induce editing of a target site specifically bound and edited by the fusion protein. As will be appreciated by the skilled artisan, the effective amount of an agent, e.g., a fusion protein, a nuclease, a cytidine deaminase or adenosine deaminase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

“Epitope,” as used herein, means an antigenic determinant. An epitope is the part of an antigen molecule that by its structure determines the specific antibody molecule that will recognize and bind it.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Graft versus host disease” (GVHD) refers to a pathological condition where transplanted cells of a donor generate an immune response against cells of the host.

“Host versus graft disease” (HVGD) refers to a pathological condition where the immune system of a host generates an immune response against transplanted cells of a donor.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immune cell” is meant a cell of the immune system capable of generating an immune response.

By “immune effector cell” is meant a lymphocyte, once activated, capable of effecting an immune response upon a target cell. A T cell is an exemplary immune effector cell.

By “immune response regulation gene” or “immune response regulator” is meant a gene that encodes a polypeptide that is involved in regulation of a immune response. An immune response regulation gene may regulate immune response in multiple mechanisms or on different levels. For example, an immune response regulation gene may inhibit or facilitate the activation of an immune cell, e.g. a T cell. An immune response regulation gene may increase or decrease the activation threshold of a immune cell. In some embodiments, the immune response regulation gene positively regulates an immune cell signal transduction pathway. In some embodiments, the immune response regulation gene negatively regulates an immune cell signal transduction pathway. In some embodiments, the immune response regulation gene encodes an antigen, an antibody, a cytokine, or a neuroendocrine. In some embodiments, the immune response regulation gene encodes a Cblb protein.

By “immunogenic gene” is meant a gene that encodes a polypeptide that is able to elicit an immune response. For example, an immunogenic gene may encode an immunogen that elicits an immune response. In some embodiments, an immunogenic gene encodes a cell surface protein. In some embodiments, an immunogenic gene encodes a cell surface antigen or a cell surface marker. In some embodiments, the cell surface marker is a T cell marker or a B cell marker. In some embodiments, an immunogenic gene encodes a CD2, CD3e, CD3 delta, CD3 gamma, TRAC, TRBC1, TRBC2, CD4, CD5, CD7, CD8, CD19, CD23, CD27, CD28, CD30, CD33, CD52, CD70, CD127, CD122, CD130, CD132, CD38, CD69, CD11a, CD58, CD99, CD103, CCR4, CCR5, CCR6, CCR9, CCR10, CXCR3, CXCR4, CLA, CD161, B2M, or CIITA polypeptide.

The term “inhibitor of base repair” or “IBR” refers to a protein that is capable in inhibiting the activity of a nucleic acid repair enzyme, for example a base excision repair enzyme. In some embodiments, the IBR is an inhibitor of inosine base excision repair. Exemplary inhibitors of base repair include inhibitors of APE1, Endo III, Endo IV, Endo V, Endo VIII, Fpg, hOGG1, hNEIL1, T7 Endo1, T4PDG, UDG, hSMUG1, and hAAG. In some embodiments, the IBR is an inhibitor of Endo V or hAAG. In some embodiments, the IBR is a catalytically inactive EndoV or a catalytically inactive hAAG.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker,” as used herein, refers to a bond (e.g., covalent bond), chemical group, or a molecule linking two molecules or moieties, e.g., two domains of a fusion protein, such as, for example, a nuclease-inactive Cas9 domain and a nucleic acid-editing domain (e.g., a cytidine deaminase, adenosine deaminase) or in the context of a chimeric antigen receptor, a linker linking a variable heavy (VH) region to a constant heavy (CH) region. In some embodiments, the linker joins two domains of a fusion protein, such as, for example, a nuclease-inactive Cas9 domain and a nucleic acid-editing domain (e.g., a cytidine deaminase, adenosine deaminase). In some embodiments, a linker joins a gRNA binding domain of an RNA-programmable nuclease, including a Cas9 nuclease domain, and the catalytic domain of a nucleic-acid editing protein. In some embodiments, a linker joins a dCas9 and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 25, 35, 45, 50, 55, 60, 60, 65, 70, 70, 75, 80, 85, 90, 90, 95, 100, 101, 102, 103, 104, 105, 110, 120, 130, 140, 150, 160, 175, 180, 190, or 200 amino acids in length. Longer or shorter linkers are also contemplated. In some embodiments, a linker comprises the amino acid sequence SGSETPGTSESATPES, which may also be referred to as the XTEN linker. In some embodiments, a linker comprises the amino acid sequence SGGS. In some embodiments, a linker comprises (SGGS)ₙ, (GGGS)ₙ, (GGGGS)ₙ, (G)ₙ, (EAAAK)ₙ, (GGS)ₙ, SGSETPGTSESATPES, or (XP)ₙ motif, or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15.

In some embodiments, the chimeric antigen receptor comprises at least one linker. The at least one linker joins, or links, a variable heavy (VH) region to a constant heavy (CH) region of the extracellular binding domain of the chimeric antigen receptor. Linkers can also link a variable light (VL) region to a variable constant (VC) region of the extracellular binding domain.

In some embodiments, the domains of the cytidine deaminase or adenosine deaminase nucleobase editor are fused via a linker that comprises the amino acid sequence of SGGSSGSETPGTSESATPESSGGS, SGGSSGGSSGSETPGTSESATPESSGGSSGGS, or GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS. In some embodiments, domains of the cytidine deaminase or adenosine deaminase nucleobase editor are fused via a linker comprising the amino acid sequence SGSETPGTSESATPES, which may also be referred to as the XTEN linker. In some embodiments, the linker is 24 amino acids in length. In some embodiments, the linker comprises the amino acid sequence SGGSSGGSSGSETPGTSESATPES. In some embodiments, the linker is 40 amino acids in length. In some embodiments, the linker comprises the amino acid sequence SGGSSGGSSGSETPGTSESATPESSGGSSGGSSGGSSGGS. In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGS SGGS. In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAP GTSTEPSEGSAPGTSESATPESGPGSEPATS.

By “marker” is meant any protein or polynucleotide having an alteration in expression level or activity that is associated with a disease or disorder.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

“Neoplasia” refers to cells or tissues exhibiting abnormal growth or proliferation. The term neoplasia encompasses cancer and solid tumors.

By “nuclear factor of activated T cells 1 (NFATc1) polypeptide” is meant a protein having at least about 85% amino acid sequence identity to NCBI Accession No. NM_172390.2 or a fragment thereof and is a component of the activated T cell DNA-binding transcription complex. An exemplary amino acid sequence is provided below.

>NP_765978.1 nuclear factor of activated T-cells, cytoplasmic 1 isoform A [Homo sapiens]

MPSTSFPVPSKFPLGPAAAVFGRGETLGPAPRAGGTMKSAEEEHYGYASS
NVSPALPLPTAHSTLPAPCHNLQTSTPGIIPPADHPSGYGAALDGGPAGY
FLSSGHTRPDGAPALESPRIEITSCLGLYHNNNQFFHDVEVEDVLPSSKR
SPSTATLSLPSLEAYRDPSCLSPASSLSSRSCNSEASSYESNYSYPYASP
QTSPWQSPCVSPKTTDPEEGFPRGLGACTLLGSPRHSPSTSPRASVTEES
WLGARSSRPASPCNKRKYSLNGRQPPYSPHHSPTPSPHGSPRVSVTDDSW
LGNTTQYTSSAIVAAINALTTDSSLDLGDGVPVKSRKTTLEQPPSVALKV
EPVGEDLGSPPPPADFAPEDYSSFQHIRKGGFCDQYLAVPQHPYQWAKPK
PLSPTSYMSPTLPALDWQLPSHSGPYELRIEVQPKSHHRAHYETEGSRGA
VKASAGGHPIVQLHGYLENEPLMLQLFIGTADDRLLRPHAFYQVHRITGK
TVSTTSHEAILSNTKVLEIPLLPENSMRAVIDCAGILKLRNSDIELRKGE
TDIGRKNTRVRLVFRVHVPQPSGRTLSLQVASNPIECSQRSAQELPLVEK
QSTDSYPVVGGKKMVLSGHNFLQDSKVIFVEKAPDGHHVWEMEAKTDRDL
CKPNSLVVEIPPFRNQRITSPVHVSFYVCNGKRKRSQYQRFTYLPANGNA
IFLTVSREHERVGCFF

By “nuclear factor of activated T cells 1 (NFATc1) polynucleotide” is meant a nucleic acid molecule encoding a NFATc1 polypeptide. The NFATc1 gene encodes a protein that is involved in in the inducible expression of cytokine genes, especially IL-2 and IL-4, in T-cells. An exemplary nucleic acid sequenced is provided below.

>NM_172390.2 Homo sapiens nuclear factor of activated T cells 1 (NFATC1), transcript variant 1, mRNA

GGCGGGCGCTCGGCGACTCGTCCCCGGGGCCCCGCGCGGGCCCGGGCAGC
AGGGGCGTGATGTCACGGCAGGGAGGGGGCGCGGGAGCCGCCGGGCCGGC
GGGGAGGCGGGGGAGGTGTTTTCCAGCTTTAAAAAGGCAGGAGGCAGAGC
GCGGCCCTGCGTCAGAGCGAGACTCAGAGGCTCCGAACTCGCCGGCGGAG
TCGCCGCGCCAGATCCCAGCAGCAGGGCGCGGGCACCGGGGCGCGGGCAG
GGCTCGGAGCCACCGCGCAGGTCCTAGGGCCGCGGCCGGGCCCCGCCACG
CGCGCACACGCCCCTCGATGACTTTCCTCCGGGGCGCGCGGCGCTGAGCC
CGGGGCGAGGGCTGTCTTCCCGGAGACCCGACCCCGGCAGCGCGGGGCGG
CCGCTTCTCCTGTGCCTCCGCCCGCCGCTCCACTCCCCGCCGCCGCCGCG
CGGATGCCAAGCACCAGCTTTCCAGTCCCTTCCAAGTTTCCACTTGGCCC
TGCGGCTGCGGTCTTCGGGAGAGGAGAAACTTTGGGGCCCGCGCCGCGCG
CCGGCGGCACCATGAAGTCAGCGGAGGAAGAACACTATGGCTATGCATCC
TCCAACGTCAGCCCCGCCCTGCCGCTCCCCACGGCGCACTCCACCCTGCC
GGCCCCGTGCCACAACCTTCAGACCTCCACACCGGGCATCATCCCGCCGG
CGGATCACCCCTCGGGGTACGGAGCAGCTTTGGACGGTGGGCCCGCGGGC
TACTTCCTCTCCTCCGGCCACACCAGGCCTGATGGGGCCCCTGCCCTGGA
GAGTCCTCGCATCGAGATAACCTCGTGCTTGGGCCTGTACCACAACAATA
ACCAGTTTTTCCACGATGTGGAGGTGGAAGACGTCCTCCCTAGCTCCAAA
CGGTCCCCCTCCACGGCCACGCTGAGTCTGCCCAGCCTGGAGGCCTACAG
AGACCCCTCGTGCCTGAGCCCGGCCAGCAGCCTGTCCTCCCGGAGCTGCA
ACTCAGAGGCCTCCTCCTACGAGTCCAACTACTCGTACCCGTACGCGTCC
CCCCAGACGTCGCCATGGCAGTCTCCCTGCGTGTCTCCCAAGACCACGGA
CCCCGAGGAGGGCTTTCCCCGCGGGCTGGGGGCCTGCACACTGCTGGGTT
CCCCGCGGCACTCCCCCTCCACCTCGCCCCGCGCCAGCGTCACTGAGGAG
AGCTGGCTGGGTGCCCGCTCCTCCAGACCCGCGTCCCCTTGCAACAAGAG
GAAGTACAGCCTCAACGGCCGGCAGCCGCCCTACTCACCCCACCACTCGC
CCACGCCGTCCCCGCACGGCTCCCCGCGGGTCAGCGTGACCGACGACTCG
TGGTTGGGCAACACCACCCAGTACACCAGCTCGGCCATCGTGGCCGCCAT
CAACGCGCTGACCACCGACAGCAGCCTGGACCTGGGAGATGGCGTCCCTG
TCAAGTCCCGCAAGACCACCCTGGAGCAGCCGCCCTCAGTGGCGCTCAAG
GTGGAGCCCGTCGGGGAGGACCTGGGCAGCCCCCCGCCCCCGGCCGACTT
CGCGCCCGAAGACTACTCCTCTTTCCAGCACATCAGGAAGGGCGGCTTCT
GCGACCAGTACCTGGCGGTGCCGCAGCACCCCTACCAGTGGGCGAAGCCC
AAGCCCCTGTCCCCTACGTCCTACATGAGCCCGACCCTGCCCGCCCTGGA
CTGGCAGCTGCCGTCCCACTCAGGCCCGTATGAGCTTCGGATTGAGGTGC
AGCCCAAGTCCCACCACCGAGCCCACTACGAGACGGAGGGCAGCCGGGGG
GCCGTGAAGGCGTCGGCCGGAGGACACCCCATCGTGCAGCTGCATGGCTA
CTTGGAGAATGAGCCGCTGATGCTGCAGCTTTTCATTGGGACGGCGGACG
ACCGCCTGCTGCGCCCGCACGCCTTCTACCAGGTGCACCGCATCACAGGG
AAGACCGTGTCCACCACCAGCCACGAGGCCATCCTCTCCAACACCAAAGT
CCTGGAGATCCCACTCCTGCCGGAGAACAGCATGCGAGCCGTCATTGACT
GTGCCGGAATCCTGAAACTCAGAAACTCCGACATTGAACTTCGGAAAGGA
GAGACGGACATCGGGAGGAAGAACACACGGGTACGGCTGGTGTTCCGCGT
TCACGTCCCGCAACCCAGCGGCCGCACGCTGTCCCTGCAGGTGGCCTCCA
ACCCCATCGAATGCTCCCAGCGCTCAGCTCAGGAGCTGCCTCTGGTGGAG
AAGCAGAGCACGGACAGCTATCCGGTCGTGGGCGGGAAGAAGATGGTCCT
GTCTGGCCACAACTTCCTGCAGGACTCCAAGGTCATTTTCGTGGAGAAAG
CCCCAGATGGCCACCATGTCTGGGAGATGGAAGCGAAAACTGACCGGGAC
CTGTGCAAGCCGAATTCTCTGGTGGTTGAGATCCCGCCATTTCGGAATCA
GAGGATAACCAGCCCCGTTCACGTCAGTTTCTACGTCTGCAACGGGAAGA
GAAAGCGAAGCCAGTACCAGCGTTTCACCTACCTTCCCGCCAACGGTAAC
GCCATCTTTCTAACCGTAAGCCGTGAACATGAGCGCGTGGGGTGCTTTTT
CTAAAGACGCAGAAACGACGTCGCCGTAAAGCAGCGTGGCGTGTTGCACA
TTTAACTGTGTGATGTCCCGTTAGTGAGACCGAGCCATCGATGCCCTGAA
AAGGAAAGGAAAAGGGAAGCTTCGGATGCATTTTCCTTGATCCCTGTTGG
GGGTGGGGGGCGGGGGTTGCATACTCAGATAGTCACGGTTATTTTGCTTC
TTGCGAATGTATAACAGCCAAGGGGAAAACATGGCTCTTCTGCTCCAAAA
AACTGAGGGGGTCCTGGTGTGCATTTGCACCCTAAAGCTGCTTACGGTGA
AAAGGCAAATAGGTATAGCTATTTTGCAGGCACCTTTAGGAATAAACTTT
GCTTTTAAGCCTGTAAAAAAAAAAAAAA

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. Optimized sequences useful in the methods of the invention are shown at FIGS. 8A-8E and 9. In some embodiments, an NLS comprises the amino acid sequence PKKKRKVEGADKRTADGSEFES PKKKRKV, KRTADGSEFESPKKKRKV, KRPAATKKAGQAKKKK, KKTELQTTNAENKTKKL, KRGINDRNFWRGENGRKTR, RKSGKIAAIVVKRPRK, PKKKRKV, or MDSLLMNRRKFLYQFKNVRWAKGRRETYLC.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (., 2′- e.g., fluororibose, ribose, 2′-deoxyribose, arabinose, and hexose); and/or modified phosphate groups (e.g., phosphorothioates and 5′-N-phosphoramidite linkages).

The term “nucleic acid programmable DNA binding protein” or “napDNAbp” refers to a protein that associates with a nucleic acid (e.g., DNA or RNA), such as a guide nucleic acid, that guides the napDNAbp to a specific nucleic acid sequence. For example, a Cas9 protein can associate with a guide RNA that guides the Cas9 protein to a specific DNA sequence that has complementary to the guide RNA. In some embodiments, the napDNAbp, the napDNAbp is a Cas9 domain, for example a nuclease active Cas9, a Cas9 nickase (nCas9), or a nuclease inactive Cas9 (dCas9). Examples of nucleic acid programmable DNA binding proteins include, without limitation, Cas9 (e.g., dCas9 and nCas9), CasX, CasY, Cpf1, Cas12b/C2c1, and Cas12c/C2c3. Other nucleic acid programmable DNA binding proteins are also within the scope of this disclosure, though they may not be specifically listed in this disclosure.

As used herein, “obtaining” as in “obtaining an agent” includes synthesizing, purchasing, or otherwise acquiring the agent.

By “Programmed cell death 1 (PDCD1 or PD-1) polypeptide” is meant a protein having at least about 85% amino acid sequence identity to NCBI Accession No. AJS10360.1 or a fragment thereof. The PD-1 protein is thought to be involved in T cell function regulation during immune reactions and in tolerance conditions. An exemplary B2M polypeptide sequence is provided below.

>AJS10360.1 programmed cell death 1 protein [Homo sapiens]

MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL

By “Programmed cell death 1 (PDCD1 or PD-1) polynucleotide” is meant a nucleic acid molecule encoding a PD-1 polypeptide. The PDCD1 gene encodes an inhibitory cell surface receptor that inhibits T-cell effector functions in an antigen-specific manner. An exemplary PDCD1 nucleic acid sequence is provided below.

AY238517.1 Homo sapiens programmed cell death 1 (PDCD1) mRNA, complete cds

ATGCAGATCCCACAGGCGCCCTGGCCAGTCGTCTGGGCGGTGCTACAACT
GGGCTGGCGGCCAGGATGGTTCTTAGACTCCCCAGACAGGCCCTGGAACC
CCCCCACCTTCTCCCCAGCCCTGCTCGTGGTGACCGAAGGGGACAACGCC
ACCTTCACCTGCAGCTTCTCCAACACATCGGAGAGCTTCGTGCTAAACTG
GTACCGCATGAGCCCCAGCAACCAGACGGACAAGCTGGCCGCCTTCCCCG
AGGACCGCAGCCAGCCCGGCCAGGACTGCCGCTTCCGTGTCACACAACTG
CCCAACGGGCGTGACTTCCACATGAGCGTGGTCAGGGCCCGGCGCAATGA
CAGCGGCACCTACCTCTGTGGGGCCATCTCCCTGGCCCCCAAGGCGCAGA
TCAAAGAGAGCCTGCGGGCAGAGCTCAGGGTGACAGAGAGAAGGGCAGAA
GTGCCCACAGCCCACCCCAGCCCCTCACCCAGGCCAGCCGGCCAGTTCCA
AACCCTGGTGGTTGGTGTCGTGGGCGGCCTGCTGGGCAGCCTGGTGCTGC
TAGTCTGGGTCCTGGCCGTCATCTGCTCCCGGGCCGCACGAGGGACAATA
GGAGCCAGGCGCACCGGCCAGCCCCTGAAGGAGGACCCCTCAGCCGTGCC
TGTGTTCTCTGTGGACTATGGGGAGCTGGATTTCCAGTGGCGAGAGAAGA
CCCCGGAGCCCCCCGTGCCCTGTGTCCCTGAGCAGACGGAGTATGCCACC
ATTGTCTTTCCTAGCGGAATGGGCACCTCATCCCCCGCCCGCAGGGGCTC
AGCTGACGGCCCTCGGAGTGCCCAGCCACTGAGGCCTGAGGATGGACACT
GCTCTTGGCCCCTCTGA

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” or “increases” is meant a negative or positive alteration, respectively, of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, more at least about 25 amino acids, and even more preferably about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, and about 100 nucleotides or about 300 nucleotides or any integer thereabout or therebetween.

The term “RNA-programmable nuclease,” and “RNA-guided nuclease” are used with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is identical or homologous to a tracrRNA as provided in Jinek et ah, Science 337:816-821(2012), the entire contents of which is incorporated herein by reference. Other examples of gRNAs (e.g., those including domain 2) can be found in U.S. Provisional Patent Application No. 61/874,682, filed Sep. 6, 2013, entitled “Switchable Cas9 Nucleases and Uses Thereof,” and U.S. Provisional Patent Application, No. 61/874,746, filed Sep. 6, 2013, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRIS PR-associated system) Cas9 endonuclease, for example, Cas9 (Csn1) from Streptococcus pyogenes (see, e.g., “Complete genome sequence of an Ml strain of Streptococcus pyogenes.” Ferretti J. J., McShan W. M., Ajdic D. J., Savic D. J., Savic G., Lyon K., Primeaux C, Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C M., Gonzales K., Chao Y., Pirzada Z. A., Eckert M. R., Vogel J., Charpentier E., Nature 471:602-607(2011).

By “specifically binds” is meant a nucleic acid molecule, polypeptide, or complex thereof (e.g., a nucleic acid programmable DNA binding protein, a guide nucleic acid, and a chimeric antigen receptor), but which does not substantially recognize and bind other molecules in a sample, for example, a biological sample. For example, a chimeric antigen receptor specifically binds to a particular marker expressed on the surface of a cell, but does not bind to other polypeptides, carbohydrates, lipids, or any other compound on the surface of the cell.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a one: embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In an embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “subject” is meant a mammal, including, but not limited to, a human or non-human mammal, such as a bovine, equine, canine, ovine, or feline. Subjects include livestock, domesticated animals raised to produce labor and to provide commodities, such as food, including without limitation, cattle, goats, chickens, horses, pigs, rabbits, and sheep.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In one embodiment, such a sequence is at least 60%, 80% or 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Because RNA-programmable nucleases (e.g., Cas9) use RNA:DNA hybridization to target DNA cleavage sites, these proteins can be targeted, in principle, to any sequence specified by the guide RNA. Methods of using RNA-programmable nucleases, such as Cas9, for site-specific cleavage (e.g., to modify a genome) are known in the art (see e.g., Cong, L. et ah, Multiplex genome engineering using CRISPR/Cas systems. Science 339, 819-823 (2013); Mali, P. et ah, RNA-guided human genome engineering via Cas9. Science 339, 823-826 (2013); Hwang, W. Y. et ah, Efficient genome editing in zebrafish using a CRISPR-Cas system. Nature biotechnology 31, 227-229 (2013); Jinek, M. et ah, RNA-programmed genome editing in human cells. eLife 2, e00471 (2013); Dicarlo, J. E. et ah, Genome engineering in Saccharomyces cerevisiae using CRISPR-Cas systems. Nucleic acids research (2013); Jiang, W. et ah RNA-guided editing of bacterial genomes using CRISPR-Cas systems. Nature biotechnology 31, 233-239 (2013); the entire contents of each of which are incorporated herein by reference).

By “tet methylcytosine dioxygenase 2 (TET2) polypeptide” is meant a protein having at least about 85% amino acid sequence identity to NCBI Accession No. FM992369.1 or a fragment thereof and having catalytic activity to convert methylcytosine to 5-hydroxymethylcytosine. Defects in the gene have been associated with myeloproliferative disorders, and the enzyme's ability to methylate cytosine contributes to transcriptional regulation. An exemplary TET2 amino acid sequence is provided below.

>CAX30492.1 tet oncogene family member 2 [Homo sapiens]

MEQDRTNHVEGNRLSPFLIPSPPICQTEPLATKLQNGSPLPERAHPEVNG
DTKWHSFKSYYGIPCMKGSQNSRVSPDFTQESRGYSKCLQNGGIKRTVSE
PSLSGLLQIKKLKQDQKANGERRNFGVSQERNPGESSQPNVSDLSDKKES
VSSVAQENAVKDFTSFSTHNCSGPENPELQILNEQEGKSANYHDKNIVLL
KNKAVLMPNGATVSASSVEHTHGELLEKTLSQYYPDCVSIAVQKTTSHIN
AINSQATNELSCEITHPSHTSGQINSAQTSNSELPPKPAAVVSEACDADD
ADNASKLAAMLNTCSFQKPEQLQQQKSVFEICPSPAENNIQGTTKLASGE
EFCSGSSSNLQAPGGSSERYLKQNEMNGAYFKQSSVFTKDSFSATTTPPP
PSQLLLSPPPPLPQVPQLPSEGKSTLNGGVLEEHHHYPNQSNTTLLREVK
IEGKPEAPPSQSPNPSTHVCSPSPMLSERPQNNCVNRNDIQTAGTMTVPL
CSEKTRPMSEHLKHNPPIFGSSGELQDNCQQLMRNKEQEILKGRDKEQTR
DLVPPTQHYLKPGWIELKAPRFHQAESHLKRNEASLPSILQYQPNLSNQM
TSKQYTGNSNMPGGLPRQAYTQKTTQLEHKSQMYQVEMNQGQSQGTVDQH
LQFQKPSHQVHFSKTDHLPKAHVQSLCGTRFHFQQRADSQTEKLMSPVLK
QHLNQQASETEPFSNSHLLQHKPHKQAAQTQPSQSSHLPQNQQQQQKLQI
KNKEEILQTFPHPQSNNDQQREGSFFGQTKVEECFHGENQYSKSSEFETH
NVQMGLEEVQNINRRNSPYSQTMKSSACKIQVSCSNNTHLVSENKEQTTH
PELFAGNKTQNLHHMQYFPNNVIPKQDLLHRCFQEQEQKSQQASVLQGYK
NRNQDMSGQQAAQLAQQRYLIHNHANVFPVPDQGGSHTQTPPQKDTQKHA
ALRWHLLQKQEQQQTQQPQTESCHSQMHRPIKVEPGCKPHACMHTAPPEN
KTWKKVTKQENPPASCDNVQQKSIIETMEQHLKQFHAKSLFDHKALTLKS
QKQVKVEMSGPVTVLTRQTTAAELDSHTPALEQQTTSSEKTPTKRTAASV
LNNFIESPSKLLDTPIKNLLDTPVKTQYDFPSCRCVEQIIEKDEGPFYTH
LGAGPNVAAIREIMEERFGQKGKAIRIERVIYTGKEGKSSQGCPIAKWVV
RRSSSEEKLLCLVRERAGHTCEAAVIVILILVWEGIPLSLADKLYSELTE
TLRKYGTLTNRRCALNEERTCACQGLDPETCGASFSFGCSWSMYYNGCKF
ARSKIPRKFKLLGDDPKEEEKLESHLQNLSTLMAPTYKKLAPDAYNNQIE
YEHRAPECRLGLKEGRPFSGVTACLDFCAHAHRDLHNMQNGSTLVCTLTR
EDNREFGGKPEDEQLHVLPLYKVSDVDEFGSVEAQEEKKRSGAIQVLSSF
RRKVRMLAEPVKTCRQRKLEAKKAAAEKLSSLENSSNKNEKEKSAPSRTK
QTENASQAKQLAELLRLSGPVMQQSQQPQPLQKQPPQPQQQQRPQQQQPH
HPQTESVNSYSASGSTNPYMRRPNPVSPYPNSSHTSDIYGSTSPMNFYST
SSQAAGSYLNSSNPMNPYPGLLNQNTQYPSYQCNGNLSVDNCSPYLGSYS
PQSQPMDLYRYPSQDPLSKLSLPPIHTLYQPRFGNSQSFTSKYLGYGNQN
MQGDGFSSCTIRPNVHHVGKLPPYPTHEMDGHFMGATSRLPPNLSNPNMD
YKNGEHHSPSHIIHNYSAAPGMFNSSLHALHLQNKENDMLSHTANGLSKM
LPALNHDRTACVQGGLHKLSDANGQEKQPLALVQGVASGAEDNDEVWSDS
EQSFLDPDIGGVAVAPTHGSILIECAKRELHATTPLKNPNRNHPTRISLV
FYQHKSMNEPKHGLALWEAKMAEKAREKEEECEKYGPDYVPQKSHGKKVK
REPAEPHETSEPTYLRFIKSLAERTMSVTTDSTVTTSPYAFTRVTGPYNR
YI

By “tet methylcytosine dioxygenase 2 (TET2) polynucleotide” is meant a nucleic acid molecule encoding a TET2 polypeptide. The TETs polypeptide encodes a methylcytosine dioxygenase and has transcription regulatory activity. An exemplary TET2 nucleic acid is presented below.

>FM992369.1 Homo sapiens mRNA for tet oncogene family member 2 (TET2 gene)

CCGTGCCATCCCAACCTCCCACCTCGCCCCCAACCTTCGCGCTTGCTCTGCTTCTTCT
CCCAGGGGTGGAGACCCGCCGAGGTCCCCGGGGTTCCCGAGGGCTGCACCCTTCCC
CGCGCTCGCCAGCCCTGGCCCCTACTCCGCGCTGGTCCGGGCGCACCACTCCCCCCG
CGCCACTGCACGGCGTGAGGGCAGCCCAGGTCTCCACTGCGCGCCCCGCTGTACGG
CCCCAGGTGCCGCCGGCCTTTGTGCTGGACGCCCGGTGCGGGGGGCTAATTCCCTGG
GAGCCGGGGCTGAGGGCCCCAGGGCGGCGGCGCAGGCCGGGGCGGAGCGGGAGGA
GGCCGGGGCGGAGCAGGAGGAGGCCCGGGCGGAGGAGGAGAGCCGGCGGTAGCGG
CAGTGGCAGCGGCGAGAGCTTGGGCGGCCGCCGCCGCCTCCTCGCGAGCGCCGCGC
GCCCGGGTCCCGCTCGCATGCAAGTCACGTCCGCCCCCTCGGCGCGGCCGCCCCGAG
ACGCCGGCCCCGCTGAGTGATGAGAACAGACGTCAAACTGCCTTATGAATATTGAT
GCGGAGGCTAGGCTGCTTTCGTAGAGAAGCAGAAGGAAGCAAGATGGCTGCCCTTT
AGGATTTGTTAGAAAGGAGACCCGACTGCAACTGCTGGATTGCTGCAAGGCTGAGG
GACGAGAACGAGGCTGGCAAACATTCAGCAGCACACCCTCTCAAGATTGTTTACTTG
CCTTTGCTCCTGTTGAGTTACAACGCTTGGAAGCAGGAGATGGGCTCAGCAGCAGCC
AATAGGACATGATCCAGGAAGAGCAAATTCAACTAGAGGGCAGCCTTGTGGATGGC
CCCGAAGCAAGCCTGATGGAACAGGATAGAACCAACCATGTTGAGGGCAACAGACT
AAGTCCATTCCTGATACCATCACCTCCCATTTGCCAGACAGAACCTCTGGCTACAAA
GCTCCAGAATGGAAGCCCACTGCCTGAGAGAGCTCATCCAGAAGTAAATGGAGACA
CCAAGTGGCACTCTTTCAAAAGTTATTATGGAATACCCTGTATGAAGGGAAGCCAGA
ATAGTCGTGTGAGTCCTGACTTTACACAAGAAAGTAGAGGGTATTCCAAGTGTTTGC
AAAATGGAGGAATAAAACGCACAGTTAGTGAACCTTCTCTCTCTGGGCTCCTTCAGA
TCAAGAAATTGAAACAAGACCAAAAGGCTAATGGAGAAAGACGTAACTTCGGGGTA
AGCCAAGAAAGAAATCCAGGTGAAAGCAGTCAACCAAATGTCTCCGATTTGAGTGA
TAAGAAAGAATCTGTGAGTTCTGTAGCCCAAGAAAATGCAGTTAAAGATTTCACCA
GTTTTTCAACACATAACTGCAGTGGGCCTGAAAATCCAGAGCTTCAGATTCTGAATG
AGCAGGAGGGGAAAAGTGCTAATTACCATGACAAGAACATTGTATTACTTAAAAAC
AAGGCAGTGCTAATGCCTAATGGTGCTACAGTTTCTGCCTCTTCCGTGGAACACACA
CATGGTGAACTCCTGGAAAAAACACTGTCTCAATATTATCCAGATTGTGTTTCCATT
GCGGTGCAGAAAACCACATCTCACATAAATGCCATTAACAGTCAGGCTACTAATGA
GTTGTCCTGTGAGATCACTCACCCATCGCATACCTCAGGGCAGATCAATTCCGCACA
GACCTCTAACTCTGAGCTGCCTCCAAAGCCAGCTGCAGTGGTGAGTGAGGCCTGTGA
TGCTGATGATGCTGATAATGCCAGTAAACTAGCTGCAATGCTAAATACCTGTTCCTT
TCAGAAACCAGAACAACTACAACAACAAAAATCAGTTTTTGAGATATGCCCATCTCC
TGCAGAAAATAACATCCAGGGAACCACAAAGCTAGCGTCTGGTGAAGAATTCTGTT
CAGGTTCCAGCAGCAATTTGCAAGCTCCTGGTGGCAGCTCTGAACGGTATTTAAAAC
AAAATGAAATGAATGGTGCTTACTTCAAGCAAAGCTCAGTGTTCACTAAGGATTCCT
TTTCTGCCACTACCACACCACCACCACCATCACAATTGCTTCTTTCTCCCCCTCCTCC
TCTTCCACAGGTTCCTCAGCTTCCTTCAGAAGGAAAAAGCACTCTGAATGGTGGAGT
TTTAGAAGAACACCACCACTACCCCAACCAAAGTAACACAACACTTTTAAGGGAAG
TGAAAATAGAGGGTAAACCTGAGGCACCACCTTCCCAGAGTCCTAATCCATCTACA
CATGTATGCAGCCCTTCTCCGATGCTTTCTGAAAGGCCTCAGAATAATTGTGTGAAC
AGGAATGACATACAGACTGCAGGGACAATGACTGTTCCATTGTGTTCTGAGAAAAC
AAGACCAATGTCAGAACACCTCAAGCATAACCCACCAATTTTTGGTAGCAGTGGAG
AGCTACAGGACAACTGCCAGCAGTTGATGAGAAACAAAGAGCAAGAGATTCTGAAG
GGTCGAGACAAGGAGCAAACACGAGATCTTGTGCCCCCAACACAGCACTATCTGAA
ACCAGGATGGATTGAATTGAAGGCCCCTCGTTTTCACCAAGCGGAATCCCATCTAAA
ACGTAATGAGGCATCACTGCCATCAATTCTTCAGTATCAACCCAATCTCTCCAATCA
AATGACCTCCAAACAATACACTGGAAATTCCAACATGCCTGGGGGGCTCCCAAGGC
AAGCTTACACCCAGAAAACAACACAGCTGGAGCACAAGTCACAAATGTACCAAGTT
GAAATGAATCAAGGGCAGTCCCAAGGTACAGTGGACCAACATCTCCAGTTCCAAAA
ACCCTCACACCAGGTGCACTTCTCCAAAACAGACCATTTACCAAAAGCTCATGTGCA
GTCACTGTGTGGCACTAGATTTCATTTTCAACAAAGAGCAGATTCCCAAACTGAAAA
ACTTATGTCCCCAGTGTTGAAACAGCACTTGAATCAACAGGCTTCAGAGACTGAGCC
ATTTTCAAACTCACACCTTTTGCAACATAAGCCTCATAAACAGGCAGCACAAACACA
ACCATCCCAGAGTTCACATCTCCCTCAAAACCAGCAACAGCAGCAAAAATTACAAA
TAAAGAATAAAGAGGAAATACTCCAGACTTTTCCTCACCCCCAAAGCAACAATGAT
CAGCAAAGAGAAGGATCATTCTTTGGCCAGACTAAAGTGGAAGAATGTTTTCATGG
TGAAAATCAGTATTCAAAATCAAGCGAGTTCGAGACTCATAATGTCCAAATGGGAC
TGGAGGAAGTACAGAATATAAATCGTAGAAATTCCCCTTATAGTCAGACCATGAAA
TCAAGTGCATGCAAAATACAGGTTTCTTGTTCAAACAATACACACCTAGTTTCAGAG
AATAAAGAACAGACTACACATCCTGAACTTTTTGCAGGAAACAAGACCCAAAACTT
GCATCACATGCAATATTTTCCAAATAATGTGATCCCAAAGCAAGATCTTCTTCACAG
GTGCTTTCAAGAACAGGAGCAGAAGTCACAACAAGCTTCAGTTCTACAGGGATATA
AAAATAGAAACCAAGATATGTCTGGTCAACAAGCTGCGCAACTTGCTCAGCAAAGG
TACTTGATACATAACCATGCAAATGTTTTTCCTGTGCCTGACCAGGGAGGAAGTCAC
ACTCAGACCCCTCCCCAGAAGGACACTCAAAAGCATGCTGCTCTAAGGTGGCATCTC
TTACAGAAGCAAGAACAGCAGCAAACACAGCAACCCCAAACTGAGTCTTGCCATAG
TCAGATGCACAGGCCAATTAAGGTGGAACCTGGATGCAAGCCACATGCCTGTATGC
ACACAGCACCACCAGAAAACAAAACATGGAAAAAGGTAACTAAGCAAGAGAATCC
ACCTGCAAGCTGTGATAATGTGCAGCAAAAGAGCATCATTGAGACCATGGAGCAGC
ATCTGAAGCAGTTTCACGCCAAGTCGTTATTTGACCATAAGGCTCTTACTCTCAAAT
CACAGAAGCAAGTAAAAGTTGAAATGTCAGGGCCAGTCACAGTTTTGACTAGACAA
ACCACTGCTGCAGAACTTGATAGCCACACCCCAGCTTTAGAGCAGCAAACAACTTCT
TCAGAAAAGACACCAACCAAAAGAACAGCTGCTTCTGTTCTCAATAATTTTATAGAG
TCACCTTCCAAATTACTAGATACTCCTATAAAAAATTTATTGGATACACCTGTCAAG
ACTCAATATGATTTCCCATCTTGCAGATGTGTAGAGCAAATTATTGAAAAAGATGAA
GGTCCTTTTTATACCCATCTAGGAGCAGGTCCTAATGTGGCAGCTATTAGAGAAATC
ATGGAAGAAAGGTTTGGACAGAAGGGTAAAGCTATTAGGATTGAAAGAGTCATCTA
TACTGGTAAAGAAGGCAAAAGTTCTCAGGGATGTCCTATTGCTAAGTGGGTGGTTCG
CAGAAGCAGCAGTGAAGAGAAGCTACTGTGTTTGGTGCGGGAGCGAGCTGGCCACA
CCTGTGAGGCTGCAGTGATTGTGATTCTCATCCTGGTGTGGGAAGGAATCCCGCTGT
CTCTGGCTGACAAACTCTACTCGGAGCTTACCGAGACGCTGAGGAAATACGGCACG
CTCACCAATCGCCGGTGTGCCTTGAATGAAGAGAGAACTTGCGCCTGTCAGGGGCTG
GATCCAGAAACCTGTGGTGCCTCCTTCTCTTTTGGTTGTTCATGGAGCATGTACTACA
ATGGATGTAAGTTTGCCAGAAGCAAGATCCCAAGGAAGTTTAAGCTGCTTGGGGAT
GACCCAAAAGAGGAAGAGAAACTGGAGTCTCATTTGCAAAACCTGTCCACTCTTAT
GGCACCAACATATAAGAAACTTGCACCTGATGCATATAATAATCAGATTGAATATG
AACACAGAGCACCAGAGTGCCGTCTGGGTCTGAAGGAAGGCCGTCCATTCTCAGGG
GTCACTGCATGTTTGGACTTCTGTGCTCATGCCCACAGAGACTTGCACAACATGCAG
AATGGCAGCACATTGGTATGCACTCTCACTAGAGAAGACAATCGAGAATTTGGAGG
AAAACCTGAGGATGAGCAGCTTCACGTTCTGCCTTTATACAAAGTCTCTGACGTGGA
TGAGTTTGGGAGTGTGGAAGCTCAGGAGGAGAAAAAACGGAGTGGTGCCATTCAGG
TACTGAGTTCTTTTCGGCGAAAAGTCAGGATGTTAGCAGAGCCAGTCAAGACTTGCC
GACAAAGGAAACTAGAAGCCAAGAAAGCTGCAGCTGAAAAGCTTTCCTCCCTGGAG
AACAGCTCAAATAAAAATGAAAAGGAAAAGTCAGCCCCATCACGTACAAAACAAA
CTGAAAACGCAAGCCAGGCTAAACAGTTGGCAGAACTTTTGCGACTTTCAGGACCA
GTCATGCAGCAGTCCCAGCAGCCCCAGCCTCTACAGAAGCAGCCACCACAGCCCCA
GCAGCAGCAGAGACCCCAGCAGCAGCAGCCACATCACCCTCAGACAGAGTCTGTCA
ACTCTTATTCTGCTTCTGGATCCACCAATCCATACATGAGACGGCCCAATCCAGTTA
GTCCTTATCCAAACTCTTCACACACTTCAGATATCTATGGAAGCACCAGCCCTATGA
ACTTCTATTCCACCTCATCTCAAGCTGCAGGTTCATATTTGAATTCTTCTAATCCCAT
GAACCCTTACCCTGGGCTTTTGAATCAGAATACCCAATATCCATCATATCAATGCAA
TGGAAACCTATCAGTGGACAACTGCTCCCCATATCTGGGTTCCTATTCTCCCCAGTCT
CAGCCGATGGATCTGTATAGGTATCCAAGCCAAGACCCTCTGTCTAAGCTCAGTCTA
CCACCCATCCATACACTTTACCAGCCAAGGTTTGGAAATAGCCAGAGTTTTACATCT
AAATACTTAGGTTATGGAAACCAAAATATGCAGGGAGATGGTTTCAGCAGTTGTAC
CATTAGACCAAATGTACATCATGTAGGGAAATTGCCTCCTTATCCCACTCATGAGAT
GGATGGCCACTTCATGGGAGCCACCTCTAGATTACCACCCAATCTGAGCAATCCAAA
CATGGACTATAAAAATGGTGAACATCATTCACCTTCTCACATAATCCATAACTACAG
TGCAGCTCCGGGCATGTTCAACAGCTCTCTTCATGCCCTGCATCTCCAAAACAAGGA
GAATGACATGCTTTCCCACACAGCTAATGGGTTATCAAAGATGCTTCCAGCTCTTAA
CCATGATAGAACTGCTTGTGTCCAAGGAGGCTTACACAAATTAAGTGATGCTAATGG
TCAGGAAAAGCAGCCATTGGCACTAGTCCAGGGTGTGGCTTCTGGTGCAGAGGACA
ACGATGAGGTCTGGTCAGACAGCGAGCAGAGCTTTCTGGATCCTGACATTGGGGGA
GTGGCCGTGGCTCCAACTCATGGGTCAATTCTCATTGAGTGTGCAAAGCGTGAGCTG
CATGCCACAACCCCTTTAAAGAATCCCAATAGGAATCACCCCACCAGGATCTCCCTC
GTCTTTTACCAGCATAAGAGCATGAATGAGCCAAAACATGGCTTGGCTCTTTGGGAA
GCCAAAATGGCTGAAAAAGCCCGTGAGAAAGAGGAAGAGTGTGAAAAGTATGGCC
CAGACTATGTGCCTCAGAAATCCCATGGCAAAAAAGTGAAACGGGAGCCTGCTGAG
CCACATGAAACTTCAGAGCCCACTTACCTGCGTTTCATCAAGTCTCTTGCCGAAAGG
ACCATGTCCGTGACCACAGACTCCACAGTAACTACATCTCCATATGCCTTCACTCGG
GTCACAGGGCCTTACAACAGATATATATGAAGATATATATGATATCACCCCCTTTTG
TTGGTTACCTCACTTGAAAAGACCACAACCAACCTGTCAGTAGTATAGTTCTCATGA
CGTGGGCAGTGGGGAAAGGTCACAGTATTCATGACAAATGTGGTGGGAAAAACCTC
AGCTCACCAGCAACAAAAGAGGTTATCTTACCATAGCACTTAATTTTCACTGGCTCC
CAAGTGGTCACAGATGGCATCTAGGAAAAGACCAAAGCATTCTATGCAAAAAGAAG
GTGGGGAAGAAAGTGTTCCGCAATTTACATTTTTAAACACTGGTTCTATTATTGGAC
GAGATGATATGTAAATGTGATCCCCCCCCCCCGCTTACAACTCTACACATCTGTGAC
CACTTTTAATAATATCAAGTTTGCATAGTCATGGAACACAAATCAAACAAGTACTGT
AGTATTACAGTGACAGGAATCTTAAAATACCATCTGGTGCTGAATATATGATGTACT
GAAATACTGGAATTATGGCTTTTTGAAATGCAGTTTTTACTGTAATCTTAACTTTTAT
TTATCAAAATAGCTACAGGAAACATGAATAGCAGGAAAACACTGAATTTGTTTGGA
TGTTCTAAGAAATGGTGCTAAGAAAATGGTGTCTTTAATAGCTAAAAATTTAATGCC
TTTATATCATCAAGATGCTATCAGTGTACTCCAGTGCCCTTGAATAATAGGGGTACC
TTTTCATTCAAGTTTTTATCATAATTACCTATTCTTACACAAGCTTAGTTTTTAAAATG
TGGACATTTTAAAGGCCTCTGGATTTTGCTCATCCAGTGAAGTCCTTGTAGGACAAT
AAACGTATATATGTACATATATACACAAACATGTATATGTGCACACACATGTATATG
TATAAATATTTTAAATGGTGTTTTAGAAGCACTTTGTCTACCTAAGCTTTGACAACTT
GAACAATGCTAAGGTACTGAGATGTTTAAAAAACAAGTTTACTTTCATTTTAGAATG
CAAAGTTGATTTTTTTAAGGAAACAAAGAAAGCTTTTAAAATATTTTTGCTTTTAGCC
ATGCATCTGCTGATGAGCAATTGTGTCCATTTTTAACACAGCCAGTTAAATCCACCA
TGGGGCTTACTGGATTCAAGGGAATACGTTAGTCCACAAAACATGTTTTCTGGTGCT
CATCTCACATGCTATACTGTAAAACAGTTTTATACAAAATTGTATGACAAGTTCATT
GCTCAAAAATGTACAGTTTTAAGAATTTTCTATTAACTGCAGGTAATAATTAGCTGC
ATGCTGCAGACTCAACAAAGCTAGTTCACTGAAGCCTATGCTATTTTATGGATCATA
GGCTCTTCAGAGAACTGAATGGCAGTCTGCCTTTGTGTTGATAATTATGTACATTGT
GACGTTGTCATTTCTTAGCTTAAGTGTCCTCTTTAACAAGAGGATTGAGCAGACTGA
TGCCTGCATAAGATGAATAAACAGGGTTAGTTCCATGTGAATCTGTCAGTTAAAAAG
AAACAAAAACAGGCAGCTGGTTTGCTGTGGTGGTTTTAAATCATTAATTTGTATAAA
GAAGTGAAAGAGTTGTATAGTAAATTAAATTGTAAACAAAACTTTTTTAATGCAATG
CTTTAGTATTTTAGTACTGTAAAAAAATTAAATATATACATATATATATATATATATA
TATATATATATATGAGTTTGAAGCAGAATTCACATCATGATGGTGCTACTCAGCCTG
CTACAAATATATCATAATGTGAGCTAAGAATTCATTAAATGTTTGAGTGATGTTCCT
ACTTGTCATATACCTCAACACTAGTTTGGCAATAGGATATTGAACTGAGAGTGAAAG
CATTGTGTACCATCATTTTTTTCCAAGTCCTTTTTTTTATTGTTAAAAAAAAAAGCAT
ACCTTTTTTCAATACTTGATTTCTTAGCAAGTATAACTTGAACTTCAACCTTTTTGTTC
TAAAAATTCAGGGATATTTCAGCTCATGCTCTCCCTATGCCAACATGTCACCTGTGTT
TATGTAAAATTGTTGTAGGTTAATAAATATATTCTTTGTCAGGGATTTAACCCTTTTA
TTTTGAATCCCTTCTATTTTACTTGTACATGTGCTGATGTAACTAAAACTAATTTTGT
AAATCTGTTGGCTCTTTTTATTGTAAAGAAAAGCATTTTAAAAGTTTGAGGAATCTTT
TGACTGTTTCAAGCAGGAAAAAAAAATTACATGAAAATAGAATGCACTGAGTTGAT
AAAGGGAAAAATTGTAAGGCAGGAGTTTGGCAAGTGGCTGTTGGCCAGAGACTTAC
TTGTAACTCTCTAAATGAAGTTTTTTTGATCCTGTAATCACTGAAGGTACATACTCCA
TGTGGACTTCCCTTAAACAGGCAAACACCTACAGGTATGGTGTGCAACAGATTGTAC
AATTACATTTTGGCCTAAATACATTTTTGCTTACTAGTATTTAAAATAAATTCTTAAT
CAGAGGAGGCCTTTGGGTTTTATTGGTCAAATCTTTGTAAGCTGGCTTTTGTCTTTTT
AAAAAATTTCTTGAATTTGTGGTTGTGTCCAATTTGCAAACATTTCCAAAAATGTTTG
CTTTGCTTACAAACCACATGATTTTAATGTTTTTTGTATACCATAATATCTAGCCCCA
AACATTTGATTACTACATGTGCATTGGTGATTTTGATCATCCATTCTTAATATTTGAT
TTCTGTGTCACCTACTGTCATTTGTTAAACTGCTGGCCAACAAGAACAGGAAGTATA
GTTTGGGGGGTTGGGGAGAGTTTACATAAGGAAGAGAAGAAATTGAGTGGCATATT
GTAAATATCAGATCTATAATTGTAAATATAAAACCTGCCTCAGTTAGAATGAATGGA
AAGCAGATCTACAATTTGCTAATATAGGAATATCAGGTTGACTATATAGCCATACTT
GAAAATGCTTCTGAGTGGTGTCAACTTTACTTGAATGAATTTTTCATCTTGATTGACG
CACAGTGATGTACAGTTCACTTCTGAAGCTAGTGGTTAACTTGTGTAGGAAACTTTT
GCAGTTTGACACTAAGATAACTTCTGTGTGCATTTTTCTATGCTTTTTTAAAAACTAG
TTTCATTTCATTTTCATGAGATGTTTGGTTTATAAGATCTGAGGATGGTTATAAATAC
TGTAAGTATTGTAATGTTATGAATGCAGGTTATTTGAAAGCTGTTTATTATTATATCA
TTCCTGATAATGCTATGTGAGTGTTTTTAATAAAATTTATATTTATTTAATGCACTCT
AAGTGTTGTCTTCCT

By “transforming growth factor receptor 2 (TGFBRII) polypeptide” is meant a protein having at least about 85% sequence identity to NCBI Accession No. ABG65632.1 or a fragment thereof and having immunosuppressive activity. An exemplary amino acid sequence is provided below.

>ABG65632.1 transforming growth factor beta receptor II [Homo sapiens]

MGRGLLRGLWPLHIVLWTRIASTIPPHVQKSVNNDMIVTDNNGAVKFPQL
CKFCDVRFSTCDNQKSCMSNCSITSICEKPQEVCVAVWRKNDENITLETV
CHDPKLPYHDFILEDAASPKCIMKEKKKPGETFFMCSCSSDECNDNIIFS
EEYNTSNPDLLLVIFQVTGISLLPPLGVAISVIIIFYCYRVNRQQKLSST
WETGKTRKLMEFSEHCAIILEDDRSDISSTCANNINHNTELLPIELDTLV
GKGRFAEVYKAKLKQNTSEQFETVAVKIFPYEEYASWKTEKDIFSDINLK
HENILQFLTAEERKTELGKQYWLITAFHAKGNLQEYLTRHVISWEDLRKL
GSSLARGIAHLHSDHTPCGRPKMPIVHRDLKSSNILVKNDLTCCLCDFGL
SLRLDPTLSVDDLANSGQVGTARYMAPEVLESRMNLENVESFKQTDVYSM
ALVLWEMTSRCNAVGEVKDYEPPFGSKVREHPCVESMKDNVLRDRGRPEI
PSFWLNHQGIQMVCETLTECWDHDPEARLTAQCVAERFSELEHLDRLSGR
SCSEEKIPEDGSLNTTK

By “transforming growth factor receptor 2 (TGFBRII) polynucleotide” is meant a nucleic acid that encodes a TGFBRII polypeptide. The TGFBRII gene encodes a transmembrane protein having serine/threonine kinase activity. An exemplary TGFBRII nucleic acid is provided below.

>M85079.1 Human TGF-beta type II receptor mRNA, complete cds

GTTGGCGAGGAGTTTCCTGTTTCCCCCGCAGCGCTGAGTTGAAGTTGAGT
GAGTCACTCGCGCGCACGGAGCGACGACACCCCCGCGCGTGCACCCGCTC
GGGACAGGAGCCGGACTCCTGTGCAGCTTCCCTCGGCCGCCGGGGGCCTC
CCCGCGCCTCGCCGGCCTCCAGGCCCCTCCTGGCTGGCGAGCGGGCGCCA
CATCTGGCCCGCACATCTGCGCTGCCGGCCCGGCGCGGGGTCCGGAGAGG
GCGCGGCGCGGAGCGCAGCCAGGGGTCCGGGAAGGCGCCGTCCGTGCGCT
GGGGGCTCGGTCTATGACGAGCAGCGGGGTCTGCCATGGGTCGGGGGCTG
CTCAGGGGCCTGTGGCCGCTGCACATCGTCCTGTGGACGCGTATCGCCAG
CACGATCCCACCGCACGTTCAGAAGTCGGTTAATAACGACATGATAGTCA
CTGACAACAACGGTGCAGTCAAGTTTCCACAACTGTGTAAATTTTGTGAT
GTGAGATTTTCCACCTGTGACAACCAGAAATCCTGCATGAGCAACTGCAG
CATCACCTCCATCTGTGAGAAGCCACAGGAAGTCTGTGTGGCTGTATGGA
GAAAGAATGACGAGAACATAACACTAGAGACAGTTTGCCATGACCCCAAG
CTCCCCTACCATGACTTTATTCTGGAAGATGCTGCTTCTCCAAAGTGCAT
TATGAAGGAAAAAAAAAAGCCTGGTGAGACTTTCTTCATGTGTTCCTGTA
GCTCTGATGAGTGCAATGACAACATCATCTTCTCAGAAGAATATAACACC
AGCAATCCTGACTTGTTGCTAGTCATATTTCAAGTGACAGGCATCAGCCT
CCTGCCACCACTGGGAGTTGCCATATCTGTCATCATCATCTTCTACTGCT
ACCGCGTTAACCGGCAGCAGAAGCTGAGTTCAACCTGGGAAACCGGCAAG
ACGCGGAAGCTCATGGAGTTCAGCGAGCACTGTGCCATCATCCTGGAAGA
TGACCGCTCTGACATCAGCTCCACGTGTGCCAACAACATCAACCACAACA
CAGAGCTGCTGCCCATTGAGCTGGACACCCTGGTGGGGAAAGGTCGCTTT
GCTGAGGTCTATAAGGCCAAGCTGAAGCAGAACACTTCAGAGCAGTTTGA
GACAGTGGCAGTCAAGATCTTTCCCTATGAGGAGTATGCCTCTTGGAAGA
CAGAGAAGGACATCTTCTCAGACATCAATCTGAAGCATGAGAACATACTC
CAGTTCCTGACGGCTGAGGAGCGGAAGACGGAGTTGGGGAAACAATACTG
GCTGATCACCGCCTTCCACGCCAAGGGCAACCTACAGGAGTACCTGACGC
GGCATGTCATCAGCTGGGAGGACCTGCGCAAGCTGGGCAGCTCCCTCGCC
CGGGGGATTGCTCACCTCCACAGTGATCACACTCCATGTGGGAGGCCCAA
GATGCCCATCGTGCACAGGGACCTCAAGAGCTCCAATATCCTCGTGAAGA
ACGACCTAACCTGCTGCCTGTGTGACTTTGGGCTTTCCCTGCGTCTGGAC
CCTACTCTGTCTGTGGATGACCTGGCTAACAGTGGGCAGGTGGGAACTGC
AAGATACATGGCTCCAGAAGTCCTAGAATCCAGGATGAATTTGGAGAATG
CTGAGTCCTTCAAGCAGACCGATGTCTACTCCATGGCTCTGGTGCTCTGG
GAAATGACATCTCGCTGTAATGCAGTGGGAGAAGTAAAAGATTATGAGCC
TCCATTTGGTTCCAAGGTGCGGGAGCACCCCTGTGTCGAAAGCATGAAGG
ACAACGTGTTGAGAGATCGAGGGCGACCAGAAATTCCCAGCTTCTGGCTC
AACCACCAGGGCATCCAGATGGTGTGTGAGACGTTGACTGAGTGCTGGGA
CCACGACCCAGAGGCCCGTCTCACAGCCCAGTGTGTGGCAGAACGCTTCA
GTGAGCTGGAGCATCTGGACAGGCTCTCGGGGAGGAGCTGCTCGGAGGAG
AAGATTCCTGAAGACGGCTCCCTAAACACTACCAAATAGCTCTTATGGGG
CAGGCTGGGCATGTCCAAAGAGGCTGCCCCTCTCACCAAA

By “T Cell Immunoreceptor with Ig and ITIM Domains (TIGIT) polypeptide” is meant a protein having at least about 85% sequence identity to NCBI Accession No. ACD74757.1 or a fragment thereof and having immunomodulatory activity. An exemplary TIGIT amino acid sequence is provided below.

>ACD74757.1 T cell immunoreceptor with Ig and ITIM domains [Homo sapiens]

MRWCLLLIWAQGLRQAPLASGMMTGTIETTGNISAEKGGSIILQCHLSST
TAQVTQVNWEQQDQLLAICNADLGWHISPSFKDRVAPGPGLGLTLQSLTV
NDTGEYFCIYHTYPDGTYTGRIFLEVLESSVAEHGARFQIPLLGAMAATL
VVICTAVIVVVALTRKKKALRIHSVEGDLRRKSAGQEEWSPSAPSPPGSC
VQAEAAPAGLCGEQRGEDCAELHDYFNVLSYRSLGNCSFFTETG

By “T Cell Immunoreceptor With Ig And ITIM Domains (TIGIT) polynucleotide” is meant a nucleic acid encoding a TIGIT polypeptide. The TIGIT gene encodes an inhibitory immune receptor that is associated with neoplasia and T cell exhaustion. An exemplary nucleic acid sequence is provided below.

>EU675310.1 Homo sapiens T cell immunoreceptor with Ig and ITIM domains (TIGIT) mRNA, complete cds

CGTCCTATCTGCAGTCGGCTACTTTCAGTGGCAGAAGAGGCCACATCTGC
TTCCTGTAGGCCCTCTGGGCAGAAGCATGCGCTGGTGTCTCCTCCTGATC
TGGGCCCAGGGGCTGAGGCAGGCTCCCCTCGCCTCAGGAATGATGACAGG
CACAATAGAAACAACGGGGAACATTTCTGCAGAGAAAGGTGGCTCTATCA
TCTTACAATGTCACCTCTCCTCCACCACGGCACAAGTGACCCAGGTCAAC
TGGGAGCAGCAGGACCAGCTTCTGGCCATTTGTAATGCTGACTTGGGGTG
GCACATCTCCCCATCCTTCAAGGATCGAGTGGCCCCAGGTCCCGGCCTGG
GCCTCACCCTCCAGTCGCTGACCGTGAACGATACAGGGGAGTACTTCTGC
ATCTATCACACCTACCCTGATGGGACGTACACTGGGAGAATCTTCCTGGA
GGTCCTAGAAAGCTCAGTGGCTGAGCACGGTGCCAGGTTCCAGATTCCAT
TGCTTGGAGCCATGGCCGCGACGCTGGTGGTCATCTGCACAGCAGTCATC
GTGGTGGTCGCGTTGACTAGAAAGAAGAAAGCCCTCAGAATCCATTCTGT
GGAAGGTGACCTCAGGAGAAAATCAGCTGGACAGGAGGAATGGAGCCCCA
GTGCTCCCTCACCCCCAGGAAGCTGTGTCCAGGCAGAAGCTGCACCTGCT
GGGCTCTGTGGAGAGCAGCGGGGAGAGGACTGTGCCGAGCTGCATGACTA
CTTCAATGTCCTGAGTTACAGAAGCCTGGGTAACTGCAGCTTCTTCACAG
AGACTGGTTAGCAACCAGAGGCATCTTCTGG

By “T Cell Receptor Alpha Constant (TRAC) polypeptide” is meant a protein having at least about 85% amino acid sequence identity to NCBI Accession No. P01848.2 or fragment thereof and having immunomodulatory activity. An exemplary amino acid sequence is provided below.

>sp|P01848.2|TRAC_HUMAN RecName: Full=T cell receptor alpha constant

IQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVE
KSFETDTNLNFQNLSVIGFRILLLKVAGFNLLMTLRLWSS

By “T Cell Receptor Alpha Constant (TRAC) polynucleotide” is meant a nucleic acid encoding a TRAC polypeptide. Exemplary TRAC nucleic acid sequences are provided below.

UCSC human genome database, Gene ENSG00000277734.8 Human T-cell receptor alpha chain (TCR-alpha)

catgctaatcctccggcaaacctctgtttcctcctcaaaaggcaggaggt
cggaaagaataaacaatgagagtcacattaaaaacacaaaatcctacgga
aatactgaagaatgagtctcagcactaaggaaaagcctccagcagctcct
gattctgagggtgaaggatagacgctgtggctctgcatgactcactagca
ctctatcacggccatattctggcagggtcagtggctccaactaacatttg
tttggtactttacagtttattaaatagatgatatatggagaagctctcat
ttattctcagaagagcctggctaggaaggtggatgaggcaccatattcat
tttgcaggtgaaattcctgagatgtaaggagctgctgtgacttgctcaag
gccttatatcgagtaaacggtagtgctggggcttagacgcaggtgttctg
atttatagttcaaaacctctatcaatgagagagcaatctcctggtaatgt
gatagatttcccaacttaatgccaacataccataaacctcccattctgct
aatgcccagcctaagttggggagaccactccagattccaagatgtacagt
ttgattgctgggccatttcccatgcctgcctttactctgccagagttata
ttgctggggttttgaagaagatcctattaaataaaagaataagcagtatt
attaagtagccctgcatttcaggtttccttgagtggcaggccaggcctgg
ccgtgaacgttcactgaaatcatggcctcttggccaagattgatagcttg
tgcctgtccctgagtcccagtccatcacgagcagctggatctaagatgct
atttcccgtataaagcatgagaccgtgacttgccagccccacagagcccc
gcccttgtccatcactggcatctggactccagcctgggttggggcaaaga
gggaaatgagatcatgtcctaaccctgatcctcttgtcccacagATATCC
AGAACCCTGACCCTGCCGTGTACCAGCTGAGAGACTCTAAATCCAGTGAC
AAGTCTGTCTGCCTATTCACCGATTTTGATTCTCAAACAAATGTGTCACA
AAGTAAGGATTCTGATGTGTATATCACAGACAAAACTGTGCTAGACATGA
GGTCTATGGACTTCAAGAGCAACAGTGCTGTGGCCTGGAGCAACAAATCT
GACTTTGCATGTGCAAACGCCTTCAACAACAGCATTATTCCAGAAGACAC
CTTCTTCCCCAGCCCAGgtaagggcagattggtgccttcgcaggctgttt
ccttgcttcaggaatggccaggttctgcccagagctctggtcaatgatgt
ctaaaactcctctgattggtggtctcggccttatccattgccaccaaaac
cctattttactaagaaacagtgagccttgttctggcagtccagagaatga
cacgggaaaaaagcagatgaagagaaggtggcaggagagggcacgtggcc
cagcctcagtctctccaactgagttcctgcctgcctgcattgctcagact
gtttgccccttactgctcttctaggcctcattctaagccccttctccaag
ttgcctctccttatttctccctgtctgccaaaaaatattcccagctcact
aagtcagtctcacgcagtcactcattaacccaccaatcactgattgtgcc
ggcacatgaatgcaccaggtgttgaagtggaggaattaaaaagtcagatg
aggggtgtgcccagaggaagcaccattctagttgggggagcccatctgtc
agctgggaaaagtccaaataacttcagattggaatgtgttttaactcagg
gttgagaaaacagctaccttcaggacaaaagtcagggaagggctctctga
agaaatgctacttgaagataccagccctaccaagggcagggagaggaccc
tatagaggcctgggacaggagctcaatgagaaaggagaagagcagcaggc
atgagttgaatgaaggaggcagggccgggtcacagggccttctaggccat
gagagggtagacagtattctaaggacgccagaaagctgttgatcggcttc
aagcaggggagggacacctaatttgcttttcttttttttttttttttttt
tttttttttttgagatggagttttgctcttgttgcccaggctggagtgca
atggtgcatcttggctcactgcaacctccgcctcccaggttcaagtgatt
ctcctgcctcagcctcccgagtagctgagattacaggcacccgccaccat
gcctggctaattttttgtatttttagtagagacagggtttcactatgttg
gccaggctggtctcgaactcctgacctcaggtgatccacccgcttcagcc
tcccaaagtgctgggattacaggcgtgagccaccacacccggcctgcttt
tcttaaagatcaatctgagtgctgtacggagagtgggttgtaagccaaga
gtagaagcagaaagggagcagttgcagcagagagatgatggaggcctggg
cagggtggtggcagggaggtaaccaacaccattcaggtttcaaaggtaga
accatgcagggatgagaaagcaaagaggggatcaaggaaggcagctggat
tttggcctgagcagctgagtcaatgatagtgccgtttactaagaagaaac
caaggaaaaaatttggggtgcagggatcaaaactttttggaacatatgaa
agtacgtgtttatactctttatggcccttgtcactatgtatgcctcgctg
cctccattggactctagaatgaagccaggcaagagcagggtctatgtgtg
atggcacatgtggccagggtcatgcaacatgtactttgtacaaacagtgt
atattgagtaaatagaaatggtgtccaggagccgaggtatcggtcctgcc
agggccaggggctctccctagcaggtgctcatatgctgtaagttccctcc
agatctctccacaaggaggcatggaaaggctgtagttgttcacctgccca
agaactaggaggtctggggtgggagagtcagcctgctctggatgctgaaa
gaatgtctgtattccttttagAAAGTTCCTGTGATGTCAAGCTGGTCGAG
AAAAGCTTTGAAACAGgtaagacaggggtctagcctgggtttgcacagga
ttgcggaagtgatgaacccgcaataaccctgcctggatgagggagtggga
agaaattagtagatgtgggaatgaatgatgaggaatggaaacagcggttc
aagacctgcccagagctgggtggggtctctcctgaatccctctcaccatc
tctgactttccattctaagcactttgaggatgagtttctagcttcaatag
accaaggactctctcctaggcctctgtattcctttcaacagctccactgt
caagagagccagagagagcttctgggtggcccagctgtgaaatttctgag
tcccttagggatagccctaaacgaaccagatcatcctgaggacagccaag
aggttttgccttattcaagacaagcaacagtactcacataggctgtgggc
aatggtcctgtctctcaagaatcccctgccactcctcacacccaccctgg
gcccatattcatttccatttgagttgttcttattgagtcatccttcctgt
ggtagcggaactcactaaggggcccatctggacccgaggtattgtgatga
taaattctgagcacctaccccatccccagaagggctcagaaataaaataa
gagccaagtctagtcggtgatcctgtcttgaaacacaatactgttggccc
tggaagaatgcacagaatctgtttgtaaggggatatgcacagaagctgca
agggacaggaggtgcaggagctgcaggcctcccccacccagcctgctctg
ccttggggaaaaccgtgggtgtgtcctgcaggccatgcaggcctgggaca
tgcaagcccataaccgctgtggcctcttggttttacagATACGAACCTAA
ACTTTCAAAACCTGTCAGTGATTGGGTTCCGAATCCTCCTCCTGAAAGTG
GCCGGGTTTAATCTGCTCATGACGCTGCGGCTGTGGTCCAGCTGAGgtga
ggggccttgaagctgggagtggggtttagggacgcgggtctctgggtgca
tcctaagctctgagagcaaacctccctgcagggtcttgcttttaagtcca
aagcctgagcccaccaaactctcctacttcttcctgttacaaattcctct
tgtgcaataataatggcctgaaacgctgtaaaatatcctcatttcagccg
cctcagttgcacttctcccctatgaggtaggaagaacagttgtttagaaa
cgaagaaactgaggccccacagctaatgagtggaggaagagagacacttg
tgtacaccacatgccttgtgttgtacttctctcaccgtgtaacctcctca
tgtcctctctccccagtacggctctcttagctcagtagaaagaagacatt
acactcatattacaccccaatcctggctagagtctccgcaccctcctccc
ccagggtccccagtcgtcttgctgacaactgcatcctgttccatcaccat
caaaaaaaaactccaggctgggtgcgggggctcacacctgtaatcccagc
actttgggaggcagaggcaggaggagcacaggagctggagaccagcctgg
gcaacacagggagaccccgcctctacaaaaagtgaaaaaattaaccaggt
gtggtgctgcacacctgtagtcccagctacttaagaggctgagatgggag
gatcgcttgagccctggaatgttgaggctacaatgagctgtgattgcgtc
actgcactccagcctggaagacaaagcaagatcctgtctcaaataataaa
aaaaataagaactccagggtacatttgctcctagaactctaccacatagc
cccaaacagagccatcaccatcacatccctaacagtcctgggtcttcctc
agtgtccagcctgacttctgttcttcctcattccagATCTGCAAGATTGT
AAGACAGCCTGTGCTCCCTCGCTCCTTCCTCTGCATTGCCCCTCTTCTCC
CTCTCCAAACAGAGGGAACTCTCCTACCCCCAAGGAGGTGAAAGCTGCTA
CCACCTCTGTGCCCCCCCGGCAATGCCACCAACTGGATCCTACCCGAATT
TATGATTAAGATTGCTGAAGAGCTGCCAAACACTGCTGCCACCCCCTCTG
TTCCCTTATTGCTGCTTGTCACTGCCTGACATTCACGGCAGAGGCAAGGC
TGCTGCAGCCTCCCCTGGCTGTGCACATTCCCTCCTGCTCCCCAGAGACT
GCCTCCGCCATCCCACAGATGATGGATCTTCAGTGGGTTCTCTTGGGCTC
TAGGTCCTGCAGAATGTTGTGAGGGGTTTATTTTTTTTTAATAGTGTTCA
TAAAGAAATACATAGTATTCTTCTTCTCAAGACGTGGGGGGAAATTATCT
CATTATCGAGGCCCTGCTATGCTGTGTATCTGGGCGTGTTGTATGTCCTG
CTGCCGATGCCTTCATTAAAATGATTTGGAAGAGCAGA

Nucleotides in lower cases above are untranslated regions or introns, and nucleotides in upper cases are exons.

>X02592.1 Human mRNA for T-cell receptor alpha chain (TCR-alpha)

TTTTGAAACCCTTCAAAGGCAGAGACTTGTCCAGCCT
AACCTGCCTGCTGCTCCTAGCTCCTGAGGCTCAGGGC
CCTTGGCTTCTGTCCGCTCTGCTCAGGGCCCTCCAGC
GTGGCCACTGCTCAGCCATGCTCCTGCTGCTCGTCCC
AGTGCTCGAGGTGATTTTTACCCTGGGAGGAACCAGA
GCCCAGTCGGTGACCCAGCTTGGCAGCCACGTCTCTG
TCTCTGAAGGAGCCCTGGTTCTGCTGAGGTGCAACTA
CTCATCGTCTGTTCCACCATATCTCTTCTGGTATGTG
CAATACCCCAACCAAGGACTCCAGCTTCTCCTGAAGT
ACACATCAGCGGCCACCCTGGTTAAAGGCATCAACGG
TTTTGAGGCTGAATTTAAGAAGAGTGAAACCTCCTTC
CACCTGACGAAACCCTCAGCCCATATGAGCGACGCGG
CTGAGTACTTCTGTGCTGTGAGTGATCTCGAACCGAA
CAGCAGTGCTTCCAAGATAATCTTTGGATCAGGGACC
AGACTCAGCATCCGGCCAAATATCCAGAACCCTGACC
CTGCCGTGTACCAGCTGAGAGACTCTAAATCCAGTGA
CAAGTCTGTCTGCCTATTCACCGATTTTGATTCTC
AAACAAATGTGTCACAAAGTAAGGATTCTGATGTGTA
TATCACAGACAAAACTGTGCTAGACATGAGGTCTATG
GACTTCAAGAGCAACAGTGCTGTGGCCTGGAGCAACAA
ATCTGACTTTGCATGTGCAAACGCCTTCAACAACAGC
ATTATTCCAGAAGACACCTTCTTCCCCAGCCCAGAAA
GTTCCTGTGATGTCAAGCTGGTCGAGAAAAGCTTTGA
AACAGATACGAACCTAAACTTTCAAAACCTGTCAGTG
ATTGGGTTCCGAATCCTCCTCCTGAAAGTGGCCGGGT
TTAATCTGCTCATGACGCTGCGGCTGTGGTCCAGCTG
AGATCTGCAAGATTGTAAGACAGCCTGTGCTCCCTCG
CTCCTTCCTCTGCATTGCCCCTCTTCTCCCTCTCCAA
ACAGAGGGAACTCTCCTACCCCCAAGGAGGTGAAAGC
TGCTACCACCTCTGTGCCCCCCCGGTAATGCCACCAA
CTGGATCCTACCCGAATTTATGATTAAGATTGCTGAA
GAGCTGCCAAACACTGCTGCCACCCCCTCTGTTCCCT
TATTGCTGCTTGTCACTGCCTGACATTCACGGCAGAG
GCAAGGCTGCTGCAGCCTCCCCTGGCTGTGCACATTC
CCTCCTGCTCCCCAGAGACTGCCTCCGCCATCCCACA
GATGATGGATCTTCAGTGGGTTCTCTTGGGCTCTAGG
TCCTGGAGAATGTTGTGAGGGGTTTATTTTTTTTTAA
TAGTGTTCATAAAGAAATACATAGTATTCTTCTTCTC
AAGACGTGGGGGGAAATTATCTCATTATCGAGGCCC
TGCTATGCTGTGTGTCTGGGCGTGTTGTATGTCCTG
CTGCCGATGCCTTCATTAAAATGATTTGGAA

By “T cell receptor beta constant 1 polypeptide (TRBC1)” is meant a protein having at least about 85% amino acid sequence identity to NCBI Accession No. P01850 or fragment thereof and having immunomodulatory activity. An exemplary amino acid sequence is provided below.

.>sp|P01850|TRBC1_HUMAN T cell receptor beta constant 1 OS═Homo sapiens OX=9606 GN=TRBC1 PE=1

SV = 4DLNKVFPPEVAVFEPSEAEISHTQKATLVCLATGFFPDHVELSW
WVNGKEVHSGVSTDPQPLKEQPALNDSRYCLSSRLRVSATFWQNPRNHFR
CQVQFYGLSENDEWTQDRAKPVTQIVSAEAWGRADCGFTSVSYQQGVLSA
TILYEILLGKATLYAVLVSALVLMAMVKRKDF

By “T cell receptor beta constant 1 polynucleotide (TRBC1)” is meant a nucleic acid encoding a TRBC1 polypeptide. An exemplary TRBC1 nucleic acid sequence is provided below.>

### X00437.1

CTGGTCTAGAATATTCCACATCTGCTCTCACTCTGCCATGGACTCCTGGA
CCTTCTGCTGTGTGTCCCTTTGCATCCTGGTAGCGAAGCATACAGATGCT
GGAGTTATCCAGTCACCCCGCCATGAGGTGACAGAGATGGGACAAGAAGT
GACTCTGAGATGTAAACCAATTTCAGGCCACAACTCCCTTTTCTGGTACA
GACAGACCATGATGCGGGGACTGGAGTTGCTCATTTACTTTAACAACAAC
GTTCCGATAGATGATTCAGGGATGCCCGAGGATCGATTCTCAGCTAAGAT
GCCTAATGCATCATTCTCCACTCTGAAGATCCAGCCCTCAGAACCCAGGG
ACTCAGCTGTGTACTTCTGTGCCAGCAGTTTCTCGACCTGTTCGGCTAAC
TATGGCTACACCTTCGGTTCGGGGACCAGGTTAACCGTTGTAGAGGACCT
GAACAAGGTGTTCCCACCCGAGGTCGCTGTGTTTGAGCCATCAGAAGCAG
AGATCTCCCACACCCAAAAGGCCACACTGGTGTGCCTGGCCACAGGCTTC
TTCCCCGACCACGTGGAGCTGAGCTGGTGGGTGAATGGGAAGGAGGTGCA
CAGTGGGGTCAGCACAGACCCGCAGCCCCTCAAGGAGCAGCCCGCCCTCA
ATGACTCCAGATACTGCCTGAGCAGCCGCCTGAGGGTCTCGGCCACCTTC
TGGCAGAACCCCCGCAACCACTTCCGCTGTCAAGTCCAGTTCTACGGGCT
CTCGGAGAATGACGAGTGGACCCAGGATAGGGCCAAACCCGTCACCCAGA
TCGTCAGCGCCGAGGCCTGGGGTAGAGCAGACTGTGGCTTTACCTCGGTG
TCCTACCAGCAAGGGGTCCTGTCTGCCACCATCCTCTATGAGATCCTGCT
AGGGAAGGCCACCCTGTATGCTGTGCTGGTCAGCGCCCTTGTGTTGATGG
CCATGGTCAAGAGAAAGGATTTCTGAAGGCAGCCCTGGAAGTGGAGTTAG
GAGCTTCTAACCCGTCATGGTTCAATACACATTCTTCTTTTGCCAGCGCT
TCTGAAGAGCTGCTCTCACCTCTCTGCATCCCAATAGATATCCCCCTATG
TGCATGCACACCTGCACACTCACGGCTGAAATCTCCCTAACCCAGGGGGA
C

By “T cell receptor beta constant 2 polypeptide (TRBC2)” is meant a protein having at least about 85% amino acid sequence identity to NCBI Accession No. A0A5B9 or fragment thereof and having immunomodulatory activity. An exemplary amino acid sequence is provided below.

.>sp|A0A5B9|TRBC2_HUMAN T cell receptor beta constant 2 OS═Homo sapiens OX=9606 GN=TRBC2 PE=1

SV = 2DLKNVFPPKVAVFEPSEAEISHTQKATLVCLATGFYPDHVELSW
WVNGKEVHSGVSTDPQPLKEQPALNDSRYCLSSRLRVSATFWQNPRNHFR
CQVQFYGLSENDEWTQDRAKPVTQIVSAEAWGRADCGFTSESYQQGVLSA
TILYEILLGKATLYAVLVSALVLMAMVKRKDSRG

By “T cell receptor beta constant 2 polynucleotide (TRBC2)” is meant a nucleic acid encoding a TRAC polypeptide. An exemplary TRBC2 nucleic acid sequence is provided below.

>NG_001333.2:655095-656583 Homo sapiens T cell receptor beta locus (TRB) on chromosome7

AGGACCTGAAAAACGTGTTCCCACCCGAGGTCGCTGTGTTTGAGCCATCA
GAAGCAGAGATCTCCCACACCCAAAAGGCCACACTGGTATGCCTGGCCAC
AGGCTTCTACCCCGACCACGTGGAGCTGAGCTGGTGGGTGAATGGGAAGG
AGGTGCACAGTGGGGTCAGCACAGACCCGCAGCCCCTCAAGGAGCAGCCC
GCCCTCAATGACTCCAGATACTGCCTGAGCAGCCGCCTGAGGGTCTCGGC
CACCTTCTGGCAGAACCCCCGCAACCACTTCCGCTGTCAAGTCCAGTTCT
ACGGGCTCTCGGAGAATGACGAGTGGACCCAGGATAGGGCCAAACCCGTC
ACCCAGATCGTCAGCGCCGAGGCCTGGGGTAGAGCAGGTGAGTGGGGCCT
GGGGAGATGCCTGGAGGAGATTAGGTGAGACCAGCTACCAGGGAAAATGG
AAAGATCCAGGTAGCGGACAAGACTAGATCCAGAAGAAAGCCAGAGTGGA
CAAGGTGGGATGATCAAGGTTCACAGGGTCAGCAAAGCACGGTGTGCACT
TCCCCCACCAAGAAGCATAGAGGCTGAATGGAGCACCTCAAGCTCATTCT
TCCTTCAGATCCTGACACCTTAGAGCTAAGCTTTCAAGTCTCCCTGAGGA
CCAGCCATACAGCTCAGCATCTGAGTGGTGTGCATCCCATTCTCTTCTGG
GGTCCTGGTTTCCTAAGATCATAGTGACCACTTCGCTGGCACTGGAGCAG
CATGAGGGAGACAGAACCAGGGCTATCAAAGGAGGCTGACTTTGTACTAT
CTGATATGCATGTGTTTGTGGCCTGTGAGTCTGTGATGTAAGGCTCAATG
TCCTTACAAAGCAGCATTCTCTCATCCATTTTTCTTCCCCTGTTTTCTTT
CAGACTGTGGCTTCACCTCCGGTAAGTGAGTCTCTCCTTTTTCTCTCTAT
CTTTCGCCGTCTCTGCTCTCGAACCAGGGCATGGAGAATCCACGGACACA
GGGGCGTGAGGGAGGCCAGAGCCACCTGTGCACAGGTGCCTACATGCTCT
GTTCTTGTCAACAGAGTCTTACCAGCAAGGGGTCCTGTCTGCCACCATCC
TCTATGAGATCTTGCTAGGGAAGGCCACCTTGTATGCCGTGCTGGTCAGT
GCCCTCGTGCTGATGGCCATGGTAAGGAGGAGGGTGGGATAGGGCAGATG
ATGGGGGCAGGGGATGGAACATCACACATGGGCATAAAGGAATCTCAGAG
CCAGAGCACAGCCTAATATATCCTATCACCTCAATGAAACCATAATGAAG
CCAGACTGGGGAGAAAATGCAGGGAATATCACAGAATGCATCATGGGAGG
ATGGAGACAACCAGCGAGCCCTACTCAAATTAGGCCTCAGAGCCCGCCTC
CCCTGCCCTACTCCTGCTGTGCCATAGCCCCTGAAACCCTGAAAATGTTC
TCTCTTCCACAGGTCAAGAGAAAGGATTCCAGAGGCTAG

As used herein “transduction” means to transfer a gene or genetic material to a cell via a viral vector.

“Transformation,” as used herein refers to the process of introducing a genetic change in a cell produced by the introduction of exogenous nucleic acid.

“Transfection” refers to the transfer of a gene or genetical material to a cell via a chemical or physical means.

By “translocation” is meant the rearrangement of nucleic acid segments between non-homologous chromosomes.

As used herein, the terms “treat,” treating,” “treatment,” and the like refer to reducing or ameliorating a disorder and/or a symptom associated therewith. It will be appreciated that, although not precluded, treating a disorder or condition does not require that the disorder, condition or symptoms associated therewith be eliminated.

The term “uracil glycosylase inhibitor” or “UGI,” as used herein, refers to a protein that is capable of inhibiting a uracil-DNA glycosylase base-excision repair enzyme. In some embodiments, the polypeptide further contains one or more (e.g., 1, 2, 3, 4, 5) Uracil glycosylase inhibitors. In some embodiments, a UGI domain comprises a wild-type UGI or a modified version thereof. In some embodiments, the UGI proteins provided herein include fragments of UGI and proteins homologous to a UGI or a UGI fragment. For example, in some embodiments, a UGI domain comprises a fragment of the amino acid sequence set forth herein below. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or 100% of an exemplary UGI sequence provided herein. In some embodiments, a UGI comprises an amino acid sequence homologous to the amino acid sequence set forth herein below, or an amino acid sequence homologous to a fragment of the amino acid sequence set forth herein below. In some embodiments, proteins comprising UGI or fragments of UGI or homologs of UGI or UGI fragments are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth herein. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth below. In some embodiments, the UGI comprises the following amino acid sequence:

>splP14739IUNGI_BPPB2 Uracil-DNA glycosylase inhibitor

MTNLSDIIEKETGKQLVIQESILMLPEEVEEVIGNKPESDILVHTAYDES
TDENVMLLT SD APE YKPW ALVIQDSNGENKIKML

The term “vector” refers to a means of introducing a nucleic acid sequence into a cell, resulting in a transformed cell. Vectors include plasmids, transposons, phages, viruses, liposomes, and episome. “Expression vectors” are nucleic acid sequences comprising the nucleotide sequence to be expressed in the recipient cell. Expression vectors may include additional nucleic acid sequences to promote and/or facilitate the expression of the of the introduced sequence such as start, stop, enhancer, promoter, and secretion sequences.

By “zeta chain of T cell receptor associated protein kinase 70 (ZAP70) polypeptide” is meant a protein having at least about 85% amino acid sequence identity to NCBI Accession No. AAH53878.1 and having kinase activity. An exemplary amino acid sequence is provided below.

>AAH53878.1 Zeta-chain (TCR) associated protein kinase 70 kDa [Homo sapiens]

MPDPAAHLPFFYGSISRAEAEEHLKLAGMADGLFLLRQCLRSLGGYVLSL
VHDVRFHHFPIERQLNGTYAIAGGKAHCGPAELCEFYSRDPDGLPCNLRK
PCNRPSGLEPQPGVFDCLRDAMVRDYVRQTWKLEGEALEQAIISQAPQVE
KLIATTAHERMPWYHSSLTREEAERKLYSGAQTDGKFLLRPRKEQGTYAL
SLIYGKTVYHYLISQDKAGKYCIPEGTKFDTLWQLVEYLKLKADGLIYCL
KEACPNSSASNASGAAAPTLPAHPSTLTHPQRRIDTLNSDGYTPEPARIT
SPDKPRPMPMDTSVYESPYSDPEELKDKKLFLKRDNLLIADIELGCGNFG
SVRQGVYRMRKKQIDVAIKVLKQGTEKADTEEMMREAQIMHQLDNPYIVR
LIGVCQAEALMLVMEMAGGGPLHKFLVGKREEIPVSNVAELLHQVSMGMK
YLEEKNFVHRDLAARNVLLVNRHYAKISDFGLSKALGADDSYYTARSAGK
WPLKWYAPECINFRKFSSRSDVWSYGVTMWEALSYGQKPYKKMKGPEVMA
FIEQGKRMECPPECPPELYALMSDCWIYKWEDRPDFLTVEQRMRACYYSL
ASKVEGPPGSTQKAEAACA

By “zeta chain of T cell receptor associated protein kinase 70 (ZAP70) polynucleotide” is meant a nucleic acid encoding a ZAP70 polypeptide. The ZAP70 gene encodes a tyrosine kinase that is involved in T cell development and lymphocyte activation. Absence of functional ZAP10 can lead to a severe combined immunodeficiency characterized by the lack of CD8+ T cells. An exemplary ZAP70 nucleic acid sequence is provided below.

>BC053878.1 Homo sapiens zeta-chain (TCR) associated protein kinase 70 kDa, mRNA (cDNA clone MGC:61743 IMAGE:5757161), complete cds

GCTTGCCGGAGCTCAGCAGACACCAGGCCTTCCGGGCAGGCCTGGCCCAC
CGTGGGCCTCAGAGCTGCTGCTGGGGCATTCAGAACCGGCTCTCCATTGG
CATTGGGACCAGAGACCCCGCAAGTGGCCTGTTTGCCTGGACATCCACCT
GTACGTCCCCAGGTTTCGGGAGGCCCAGGGGCGATGCCAGACCCCGCGGC
GCACCTGCCCTTCTTCTACGGCAGCATCTCGCGTGCCGAGGCCGAGGAGC
ACCTGAAGCTGGCGGGCATGGCGGACGGGCTCTTCCTGCTGCGCCAGTGC
CTGCGCTCGCTGGGCGGCTATGTGCTGTCGCTCGTGCACGATGTGCGCTT
CCACCACTTTCCCATCGAGCGCCAGCTCAACGGCACCTACGCCATTGCCG
GCGGCAAAGCGCACTGTGGACCGGCAGAGCTCTGCGAGTTCTACTCGCGC
GACCCCGACGGGCTGCCCTGCAACCTGCGCAAGCCGTGCAACCGGCCGTC
GGGCCTCGAGCCGCAGCCGGGGGTCTTCGACTGCCTGCGAGACGCCATGG
TGCGTGACTACGTGCGCCAGACGTGGAAGCTGGAGGGCGAGGCCCTGGAG
CAGGCCATCATCAGCCAGGCCCCGCAGGTGGAGAAGCTCATTGCTACGAC
GGCCCACGAGCGGATGCCCTGGTACCACAGCAGCCTGACGCGTGAGGAGG
CCGAGCGCAAACTTTACTCTGGGGCGCAGACCGACGGCAAGTTCCTGCTG
AGGCCGCGGAAGGAGCAGGGCACATACGCCCTGTCCCTCATCTATGGGAA
GACGGTGTACCACTACCTCATCAGCCAAGACAAGGCGGGCAAGTACTGCA
TTCCCGAGGGCACCAAGTTTGACACGCTCTGGCAGCTGGTGGAGTATCTG
AAGCTGAAGGCGGACGGGCTCATCTACTGCCTGAAGGAGGCCTGCCCCAA
CAGCAGTGCCAGCAACGCCTCAGGGGCTGCTGCTCCCACACTCCCAGCCC
ACCCATCCACGTTGACTCATCCTCAGAGACGAATCGACACCCTCAACTCA
GATGGATACACCCCTGAGCCAGCACGCATAACGTCCCCAGACAAACCGCG
GCCGATGCCCATGGACACGAGCGTGTATGAGAGCCCCTACAGCGACCCAG
AGGAGCTCAAGGACAAGAAGCTCTTCCTGAAGCGCGATAACCTCCTCATA
GCTGACATTGAACTTGGCTGCGGCAACTTTGGCTCAGTGCGCCAGGGCGT
GTACCGCATGCGCAAGAAGCAGATCGACGTGGCCATCAAGGTGCTGAAGC
AGGGCACGGAGAAGGCAGACACGGAAGAGATGATGCGCGAGGCGCAGATC
ATGCACCAGCTGGACAACCCCTACATCGTGCGGCTCATTGGCGTCTGCCA
GGCCGAGGCCCTCATGCTGGTCATGGAGATGGCTGGGGGCGGGCCGCTGC
ACAAGTTCCTGGTCGGCAAGAGGGAGGAGATCCCTGTGAGCAATGTGGCC
GAGCTGCTGCACCAGGTGTCCATGGGGATGAAGTACCTGGAGGAGAAGAA
CTTTGTGCACCGTGACCTGGCGGCCCGCAACGTCCTGCTGGTTAACCGGC
ACTACGCCAAGATCAGCGACTTTGGCCTCTCCAAAGCACTGGGTGCCGAC
GACAGCTACTACACTGCCCGCTCAGCAGGGAAGTGGCCGCTCAAGTGGTA
CGCACCCGAATGCATCAACTTCCGCAAGTTCTCCAGCCGCAGCGATGTCT
GGAGCTATGGGGTCACCATGTGGGAGGCCTTGTCCTACGGCCAGAAGCCC
TACAAGAAGATGAAAGGGCCGGAGGTCATGGCCTTCATCGAGCAGGGCAA
GCGGATGGAATGCCCACCAGAGTGTCCACCCGAACTGTACGCACTCATGA
GTGACTGCTGGATCTACAAGTGGGAGGATCGCCCCGACTTCCTGACCGTG
GAGCAGCGCATGCGAGCCTGTTACTACAGCCTGGCCAGCAAGGTGGAAGG
GCCCCCAGGCAGCACACAGAAGGCTGAGGCTGCCTGTGCCTGAGCTCCCG
CTGCCCAGGGGAGCCCTCCACACCGGCTCTTCCCCACCCTCAGCCCCACC
CCAGGTCCTGCAGTCTGGCTGAGCCCTGCTTGGTTGTCTCCACACACAGC
TGGGCTGTGGTAGGGGGTGTCTCAGGCCACACCGGCCTTGCATTGCCTGC
CTGGCCCCCTGTCCTCTCTGGCTGGGGAGCAGGGAGGTCCGGGAGGGTGC
GGCTGTGCAGCCTGTCCTGGGCTGGTGGCTCCCGGAGGGCCCTGAGCTGA
GGGCATTGCTTACACGGATGCCTTCCCCTGGGCCCTGACATTGGAGCCTG
GGCATCCTCAGGTGGTCAGGCGTAGATCACCAGAATAAACCCAGCTTCCC
TCTTGAAAAAAAAAAAAAAAAAAAAAAAAAAAAAAAAAAAAAAAAAAAAA
AAAAAAAAAAAAAAAAAA

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1B are illustrations of three proteins that impact T cell function. FIG. 1A is an illustration of the TRAC protein, which is a key component in graft versus host disease. FIG. 1B is an illustration of the B2M protein, a component of the MHC class 1 antigen presenting complex present on nucleated cells that can be recognized by a host's CD8+ T cells. FIG. 1C is an illustration of T cell signaling that leads to expression of the PDCD1 gene, and the resulting PD-1 protein acts to inhibit the T cell signaling.

FIG. 2 is a graph of the percentage of cells with knocked down expression of target genes after base editing. “EP” denotes electroporation.

FIG. 3 is a graph of the percentages of the observed types of genetic modification in untransduced cells or in cells transduced with a BE4 base editing system or a Cas9 nuclease.

FIG. 4 is a graph depicting target nucleotide modification percentage as measured by percentage of cells that are negative for target protein expression as determined by flow cytometry (FC) in cells transduced with BE4 and sgRNAs directing BE4 to splice site acceptors (SA) or donors (SD) or that generate a STOP codon. Control cells were mock electroporated (EP).

FIG. 5 is a diagram of the BE4 system disrupting splice site acceptors (SA), splice donors (SD), or generate STOP codons.

FIG. 6 is a chart summarizing off-target binding sites of sgRNAs employed to disrupt target genes.

FIG. 7 is a graph summarizing flow cytometry (FC) data of the percentage of cells edited with BE4 or Cas9 that exhibit reduced protein expression. Cells were either gated to B2M or CD3, the latter being a proxy for TRAC expression.

FIG. 8A is a scatter plot of FACS data of unedited control cells. FIG. 8B is a scatter plot of FACS data of cells that have been edited at the B2M, TRAC, and PD1 loci.

FIG. 9 is a graph illustrating the effectiveness of the base editing techniques described herein to modify specific genes that can negatively impact CAR-T immunotherapy.

FIG. 10 is a diagram depicting a droplet digital PCR (ddPCR) protocol to detect and quantify gene modifications and translocations.

FIG. 11 presents two graphs showing the data generated from next generation sequencing (NGS) analysis or ddPCR of cells edited using either the BE4 system or the Cas9 system.

FIG. 12 is a schematic diagram that illustrates the role Cbl-b plays in suppressing T cell activation.

FIG. 13 is a graph depicting the efficiency of Cbl-b knockdown by disruption of splice sites. SA=Splice Acceptor; SD=Splice Donor; STOP—generated STOP codon; 2° Only=secondary antibody only; C373 refers to a loss of function variant (C373R); RL1-A::APC-A=laser; ICS=intracellular staining.

FIG. 14 is a graph illustrating the rate of Cas12b-mediated indels in the GRIN2B and DNMT1 genes in T cells. EP denotes electroporation.

FIG. 15 is a graph summarizing fluorescence assisted cell sorting (FACS) data of cells transduced via electroporation (EP) with bvCas12b and guide RNAs specific for TRAC, GRIN2B, and DNMT1 and gated for CD3.

FIG. 16 is a scatter plot of fluorescence assisted cell sorting data of cells transduced CAR-P2A-mCherry lentivirus demonstrating CAR expression.

FIG. 17 is a scatter plot of fluorescence assisted cell sorting data demonstrating CAR expression in cells transduced with a poly(1,8-octanediol citrate) (POC) lentiviral vector.

FIG. 18 is graph showing that BE4 produced efficient, durable gene knockout with high product purity.

FIG. 19A is a representative FACS analysis showing loss of surface expression of a protein due to gene knockout by BE4 or spCas9. FIG. 19B is a graph show that gene knockout by BE4 or spCas9 produces loss of B2M surface expression.

FIG. 20 is a schematic depicting the locations of B2M, TRAC, and PD-1 target sites. Translocations can be detected when B2M, TRAC, and PD-1 sequences recombine.

FIG. 21 is a graph showing that multiplexed base editing does not significantly impair cell expansion.

FIG. 22 is a graph showing that BE4 generated triple-edited T cells with similar on-target editing efficiency and cellular phenotype as spCas9.

FIG. 23 depicts flow cytometry analysis showing the generation of triple-edited CD3⁻, B2M⁻, PD1⁻ T cells.

FIG. 24 depicts flow cytometry analysis showing the CAR expression in BE4 and Cas9 edited cells.

FIG. 25 is a graph showing CAR-T cell killing or antigen positive cells.

FIG. 26 are graphs showing that Cas12b and BE4 can be paired for efficient multiplex editing in T cells.

FIG. 27 is a graph showing that Cas12b can direct insertion of a chimeric antigen receptor (CAR) into a locus by introducing into a cell a double-stranded DNA template encoding the CAR in the presence of a Cas12 nuclease and an sgRNA targeting the locus.

FIGS. 28A and 28B are graphs showing protein knockdown (% Negative) using base editing targeting the genes indicated in the figures as determined by flow cytometry, gated with respect to an unedited control. The figures represent results from replicate experiments. Bars for each set of conditions are presented in the order (from left to right) as listed in the key (top to bottom). The identity of each bar in the grouping of eight bar graphs correspond to, from left to right, CD3, CD7, CD52, PD1, B2M CD2, HLADR (CIITA surrogate), and CD5.

### DETAILED DESCRIPTION OF THE INVENTION

The present invention features genetically modified immune cells having enhanced anti-neoplasia activity, resistance to immune suppression, and decreased risk of eliciting a graft versus host reaction or a host versus graft reaction, or a combination thereof. The present invention also features methods for producing and using these modified immune cells (e.g., immune effector cells, such as T cells).

In one embodiment, a subject having or having a propensity to develop graft versus host disease (GVHD) is administered a CAR-T cell that lacks or has reduced levels of functional TRAC. In one embodiment, a subject having or having a propensity to develop host versus graft disease (HVGD) is administered a CAR-T cell that lacks or has reduced levels of functional beta2 microglobulin (B2M).

The modification of immune effector cells to express chimeric antigen receptors and to knockout or knockdown specific genes to diminish the negative impact that their expression can have on immune cell function is accomplished using a base editor system comprising a cytidine deaminase or adenosine deaminase as described herein.

Autologous, patient-derived chimeric antigen receptor-T cell (CAR-T) therapies have demonstrated remarkable efficacy in treating some hematologic cancers. While these products have led to significant clinical benefit for patients, the need to generate individualized therapies creates substantial manufacturing challenges and financial burdens. Allogeneic CAR-T therapies were developed as a potential solution to these challenges, having similar clinical efficacy profiles to autologous products while treating many patients with cells derived from a single healthy donor, thereby substantially reducing cost of goods and lot-to-lot variability.

Most first-generation allogeneic CAR-Ts use nucleases to introduce two or more targeted genomic DNA double strand breaks (DSBs) in a target T cell population, relying on error-prone DNA repair to generate mutations that knock out target genes in a semi-stochastic manner. Such nuclease-based gene knockout strategies aim to reduce the risk of graft-versus-host-disease and host rejection of CAR-Ts. However, the simultaneous induction of multiple DSBs results in a final cell product containing large-scale genomic rearrangements such as balanced and unbalanced translocations, and a relatively high abundance of local rearrangements including inversions and large deletions. Furthermore, as increasing numbers of simultaneous genetic modifications are made by induced DSBs, considerable genotoxicity is observed in the treated cell population. This has the potential to significantly reduce the cell expansion potential from each manufacturing run, thereby decreasing the number of patients that can be treated per healthy donor.

Base editors (BEs) are a class of emerging gene editing reagents that enable highly efficient, user-defined modification of target genomic DNA without the creation of DSBs. Here, an alternative means of producing allogeneic CAR-T cells is proposed by using base editing technology to reduce or eliminate detectable genomic rearrangements while also improving cell expansion. As shown herein, in contrast to a nuclease-only editing strategy, concurrent modification of multiple gene loci, for example, three, four, five, six, seven, eight, night, ten, or more genetic loci by base editing produces highly efficient gene knockouts with no detectable translocation events.

In some embodiments, at least 1, 2, 3, 4, 5, 6, 7, 8, or more genes or regulatory elements thereof are modified in an immune cell with the base editing compositions and methods provided herein. In some embodiments, the at least 1, 2, 3, 4, 5, 6, 7, 8, or more genes or regulatory elements thereof comprise one or more genes selected from CD3e, CD3 delta, CD3 gamma, TRAC, TRBC1, and TRBC2. In some embodiments, the at least 1, 2, 3, 4, 5, 6, 7, 8, or more genes or regulatory elements thereof comprise one or more genes selected from CD3e, CD3 delta, CD3 gamma, TRAC, TRBC1, and TRBC2, CD7, and CD52. In some embodiments, the at least 1, 2, 3, 4, 5, 6, 7, 8, or more genes or regulatory elements thereof comprise one or more genes selected from CD3e, CD3 delta, CD3 gamma, TRAC, TRBC1, TRBC2, CD2, CD5, CD7, and CD52. In some embodiments, the at least 1, 2, 3, 4, 5, 6, 7, 8, or more genes or regulatory elements thereof comprise one or more genes selected from TRAC, CD7, and CD52. In some embodiments, the at least 1, 2, 3, 4, 5, 6, 7, 8, or more genes or regulatory elements thereof comprise one or more genes selected from TRAC, CD2, CD5, CD7, and CD52. In some embodiments, the at least 1, 2, 3, 4, 5, 6, 7, 8, or more genes or regulatory elements thereof comprise one or more genes selected from CD2, CD3 epsilon, CD3 gamma, CD3 delta, CD4, CD5, CD7, CD30, CD33, CD52, CD70, and CIITA. In some embodiments, the at least 1, 2, 3, 4, 5, 6, 7, 8, or more genes or regulatory elements thereof are selected from CD2, CD3 epsilon, CD3 gamma, CD3 delta, CD4, CD5, CD7, CD30, CD33, CD52, CD70, and CIITA. In some embodiments, the at least 1, 2, 3, 4, 5, 6, 7, 8, or more genes or regulatory elements thereof comprise one or more genes selected from CD2, CD3 epsilon, CD3 gamma, CD3 delta, CD4, CD5, CD7, CD30, CD33, CD52, CD70, and CIITA. In some embodiments, the at least 1, 2, 3, 4, 5, 6, 7, 8, or more genes or regulatory elements thereof are selected from ACAT1, ACLY, ADORA2A, AXL, B2M, BATF, BCL2L11, BTLA, CAMK2D, cAMP, CASP8, Cblb, CCR5, CD2, CD3D, CD3E, CD3G, CD4, CD5, CD7, CD8A, CD33, CD38, CD52, CD70, CD82, CD86, CD96, CD123, CD160, CD244, CD276, CDK8, CDKN1B, Chi311, CIITA, CISH, CSF2CSK, CTLA-4, CUL3, Cyp11a1, DCK, DGKA, DGKZ, DHX37, ELOB (TCEB2), ENTPD1 (CD39), FADD, FAS, GATA3, IL6, IL6R, IL10, IL10RA, IRF4, IRF8, JUNB, Lag3, LAIR-1 (CD305), LDHA, LIF, LYN, MAP4K4, MAPK14, MCJ, MEF2D, MGAT5, NR4A1, NR4A2, NR4A3, NT5E (CD73), ODC1, OTULINL (FAM105A), PAG1, PDCD1, PDIA3, PHD1 (EGLN2), PHD2 (EGLN1), PHD3 (EGLN3), PIK3CD, PIKFYVE, PPARa, PPARd, PRDMI1, PRKACA, PTEN, PTPN2, PTPN6, PTPN11, PVRIG (CD112R), RASA2, RFXANK, SELPG/PSGL1, SIGLEC15, SLA, SLAMF7, SOCS1, Spry1, Spry2, STK4, SUV39, H1TET2, TGFbRII, TIGIT, Tim-3, TMEM222, TNFAIP3, TNFRSF8 (CD30), TNFRSF10B, TOX, TOX2, TRAC, TRBC1, TRBC2, UBASH3A, VHL, VISTA, XBP1, YAP1, and ZC3H12A. In some embodiments, at least 8 genes selected from CD2, CD3 epsilon, CD3 gamma, CD3 delta, CD4, CD5, CD7, CD30, CD33, CD52, CD70, and CIITA or regulatory elements thereof are modified with the base editing compositions and methods provided herein.

In one aspect, provided herein is a universal CAR-T cell. In some embodiments, the CAR-T cell described herein is an allogeneic cell. In some embodiments, the universal CAR-T cell is an allogeneic T cell that can be used to express a desired CAR, and can be universally applicable, irrespective of the donor and the recipient's immunogenic compatibility. An allogenic immune cell may be derived from one or more donors. In certain embodiments, the allogenic immune cell is derived from a single human donor. For example, the allogenic T cell may be derived from PBMCs of a single healthy human donor. In certain embodiments, the allogenic immune cell is derived from multiple human donors. In some embodiments, an universal CAR-T cell may be generated, as described herein by using gene modification to introduce concurrent edits at multiple gene loci, for example, three, four, five, six, seven, eight, nine, ten or more genetic loci. A modification, or concurrent modifications as described herein may be a genetic editing, such as a base editing, generated by a base editor. The base editor may be a C base editor or A base editor. As is discussed herein, base editing may be used to achieve a gene disruption, such that the gene is not expressed. A modification by base editing may be used to achieve a reduction in gene expression. In some embodiments base editor may be used to introduce a genetic modification such that the edited gene does not generate a structurally or functionally viable protein product. In some embodiments, a modification, such as the concurrent modifications described herein may comprise a genetic editing, such as base editing, such that the expression or functionality of the gene product is altered in any way. For example, the expression of the gene product may be enhanced or upregulated as compared to baseline expression levels. In some embodiments the activity or functionality of the gene product may be upregulated as a result of the base editing, or multiple base editing events acting in concert.

In some embodiments, generation of universal CAR-T cell may be advantageous over autologous T cell (CAR-T), which may be difficult to generate for an urgent use. Allogeneic approaches are preferred over autologous cell preparation for a number of situations related to uncertainty of engineering autologous T cells to express a CAR and finally achieving the desired cellular products for a transplant at the time of medical emergency. However, for allogeneic T cells, or “off-the-shelf” T cells, it is important to carefully negotiate the host's reactivity to the CAR-T cells (HVGD) as well as the allogeneic T cell's potential hostility towards a host cell (GVHD). Given the scenario, base editing can be successfully used to generate multiple simultaneous gene editing events, such that (a) it is possible to generate a platform cell type that is devoid of or expresses low amounts of an endogenous T cell receptor, for example, a TCR alpha chain (such a via base editing of TRAC), or a TCR beta chain (such a through base editing of TRBC1/TRBC2); (b) it is possible to reduce or down regulate expression of antigens that may be incompatible to a host tissue system and vice versa.

In some embodiments, the methods described herein can be used to generate an autologous T cell expressing a CAR-T.

In some embodiment, multiple base editing events can be accomplished in a single electroporation event, thereby reducing electroporation event associated toxicity. Any known methods for incorporation of exogenous genetic material into a cell may be used to replace electroporation, and such methods known in the art are hereby contemplated for use in any of the methods described herein.

In one experiment, the base editor BE4 demonstrated high efficiency multiplex base editing of three cell surface targets in T cells (TRAC, B2M, and PD-1), knocking out gene expression by 95%, 95% and 88%, respectively, in a single electroporation to generate cell populations with high percentages of cells with reduced protein expression of B2M and CD3. Editing each of these genes may be useful in the creation of CAR-T cell therapies with improved therapeutic properties. Each of the genes was silenced by a single targeted base change (C to T) without the creation of double strand breaks. As a result, the BE4-treated cells also did not show any measurable translocations (large-scale genomic rearrangements), whereas cells receiving the same three edits with a nuclease did show detectable genomic rearrangements.

Thus, coupling nuclease-based knockout of the TRAC gene with simultaneous BE-mediated knockout of two additional genes yields a homogeneous allogeneic T cell population with minimal genomic rearrangements. In some embodiments, the simultaneous BE mediated knockout or knockdown, or a combination thereof, may be performed in 2 additional genes, or 3 additional genes, or 4 additional genes, or 5 additional genes, or 6 additional genes, or 7 additional genes, or 8 additional genes, or 9 additional genes, or 10 additional genes, or 11 additional genes, or 12 additional genes, or more, to yield a homogenous allogeneic T cell population with minimal genomic rearrangements, and enabling targeted insertion of a CAR transgene at the TRAC locus. In some embodiments, the disclosure provides three simultaneous gene knockouts or knockdowns, by base editing along with a CAR transgene at the TRAC locus. In some embodiments, the disclosure provides four simultaneous gene knockouts or knockdowns, by base editing along with a CAR transgene at the TRAC locus. In some embodiments, the disclosure provides five simultaneous gene knockouts or knockdowns, by base editing along with a CAR transgene at the TRAC locus. In some embodiments, the disclosure provides six simultaneous gene knockouts or knockdowns, by base editing along with a CAR transgene at the TRAC locus. In some embodiments, the disclosure provides seven simultaneous gene knockouts or knockdowns, by base editing along with a CAR transgene at the TRAC locus. In some embodiments, the disclosure provides eight simultaneous gene knockouts or knockdowns, by base editing along with a CAR transgene at the TRAC locus. In some embodiments, the disclosure provides nine simultaneous gene knockouts or knockdowns, by base editing along with a CAR transgene at the TRAC locus. In some embodiments, the disclosure provides ten simultaneous gene knockouts or knockdowns, by base editing along with a CAR transgene at the TRAC locus. In some embodiments, the disclosure provides eleven simultaneous gene knockouts or knockdowns, by base editing along with a CAR transgene at the TRAC locus. In some embodiments, the disclosure provides twelve simultaneous gene knockouts or knockdowns, by base editing along with a CAR transgene at the TRAC locus. In some embodiments, the disclosure provides thirteen simultaneous gene knockouts or knockdowns, by base editing along with a CAR transgene at the TRAC locus. In some embodiments, the disclosure provides fourteen simultaneous gene knockouts or knockdowns, by base editing along with a CAR transgene at the TRAC locus. In some embodiments, the disclosure provides fifteen simultaneous gene knockouts or knockdowns, by base editing along with a CAR transgene at the TRAC locus. In some embodiments, the disclosure provides sixteen simultaneous gene knockouts or knockdowns, by base editing along with a CAR transgene at the TRAC locus. In some embodiments, the disclosure provides seventeen simultaneous gene knockouts or knockdowns, by base editing along with a CAR transgene at the TRAC locus. In some embodiments, the disclosure provides eighteen simultaneous gene knockouts or knockdowns, by base editing along with a CAR transgene at the TRAC locus. In some embodiments, the disclosure provides nineteen simultaneous gene knockouts or knockdowns, by base editing along with a CAR transgene at the TRAC locus. In some embodiments, the disclosure provides twenty simultaneous gene knockouts or knockdowns, by base editing along with a CAR transgene at the TRAC locus. Taken together, this demonstrates that base editing alone or in combination with a single nuclease knockout and CAR insertion is a useful strategy for generating allogeneic T cells with minimal genomic rearrangements compared to nuclease-alone approaches. This method addresses known limitations of multiplex-edited T cell products and are a promising development towards the next generation of precision cell based therapies.

### Chimeric Antigen Receptor and CAR-T Cells

The invention provides immune cells modified using nucleobase editors described herein that express chimeric antigen receptors. Modification of immune cells to express a chimeric antigen receptor can enhance an immune cell's immunoreactive activity, wherein the chimeric antigen receptor has an affinity for an epitope on an antigen, wherein the antigen is associated with an altered fitness of an organism. For example, the chimeric antigen receptor can have an affinity for an epitope on a protein expressed in a neoplastic cell. Because the CAR-T cells can act independently of major histocompatibility complex (MHC), activated CAR-T cells can kill the neoplastic cell expressing the antigen. The direct action of the CAR-T cell evades neoplastic cell defensive mechanisms that have evolved in response to MHC presentation of antigens to immune cells.

In some embodiments, the invention provides immune effector cells that express chimeric antigen receptors that target B cells involved in an autoimmune response (e.g., B cells of a subject that express antibodies generated against the subject's own tissues).

Some embodiments comprise autologous immune cell immunotherapy, wherein immune cells are obtained from a subject having a disease or altered fitness characterized by cancerous or otherwise altered cells expressing a surface marker. The obtained immune cells are genetically modified to express a chimeric antigen receptor and are effectively redirected against specific antigens. Thus, in some embodiments, immune cells are obtained from a subject in need of CAR-T immunotherapy. In some embodiments, these autologous immune cells are cultured and modified shortly after they are obtained from the subject. In other embodiments, the autologous cells are obtained and then stored for future use. This practice may be advisable for individuals who may be undergoing parallel treatment that will diminish immune cell counts in the future. In allogeneic immune cell immunotherapy, immune cells can be obtained from a donor other than the subject who will be receiving treatment. The immune cells, after modification to express a chimeric antigen receptor, are administered to a subject for treating a neoplasia. In some embodiments, immune cells to be modified to express a chimeric antigen receptor can be obtained from pre-existing stock cultures of immune cells.

Immune cells and/or immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD25+ is used as a marker to select regulatory T cells. In another embodiment, the invention provides T cells that have targeted gene knockouts at the TCR constant region (TRAC), which is responsible for TCRαβ surface expression. TCR alphabeta-deficient CAR T cells are compatible with allogeneic immunotherapy (Qasim et al., Sci. Transl. Med. 9, eaaj2013 (2017); Valton et al., Mol Ther. 2015 September; 23(9): 1507-1518). If desired, residual TCRalphabeta T cells are removed using CliniMACS magnetic bead depletion to minimize the risk of GVHD. In another embodiment, the invention provides donor T cells selected ex vivo to recognize minor histocompatibility antigens expressed on recipient hematopoietic cells, thereby minimizing the risk of graft-versus-host disease (GVHD), which is the main cause of morbidity and mortality after transplantation (Warren et al., Blood 2010; 115(19):3869-3878). Another technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD45 gating strategy is employed. In some embodiments, a gating strategy for other markers specific to an immune effector cell is employed instead of, or in combination with, the CD45 gating strategy.

The immune effector cells contemplated in the invention are effector T cells. In some embodiments, the effector T cell is a naïve CD8⁺ T cell, a cytotoxic T cell, or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, the immune effector cell is any other subset of T cells. The modified immune effector cell may express, in addition to the chimeric antigen receptor, an exogenous cytokine, a different chimeric receptor, or any other agent that would enhance immune effector cell signaling or function. For example, coexpression of the chimeric antigen receptor and a cytokine may enhance the CAR-T cell's ability to lyse a target cell.

Chimeric antigen receptors as contemplated in the present invention comprise an extracellular binding domain, a transmembrane domain, and an intracellular domain. Binding of an antigen to the extracellular binding domain can activate the CAR-T cell and generate an effector response, which includes CAR-T cell proliferation, cytokine production, and other processes that lead to the death of the antigen expressing cell. In some embodiments of the present invention, the chimeric antigen receptor further comprises a linker.

The extracellular binding domain of a chimeric antigen receptor contemplated herein comprises an amino acid sequence of an antibody, or an antigen binding fragment thereof, that has an affinity for a specific antigen. In various embodiments, the CAR specifically binds 5T4. Exemplary anti-5T4 CARs include, without limitation, CART-5T4 (Oxford BioMedica plc) and UCART-5T4 (Cellectis SA).

In various embodiments, the CAR specifically binds Alpha-fetoprotein. Exemplary anti-Alpha-fetoprotein CARs include, without limitation, ET-1402 (Eureka Therapeutics Inc). In various embodiments, the CAR specifically binds Axl. Exemplary anti-Axl CARs include, without limitation, CCT-301-38 (F1 Oncology Inc). In various embodiments, the CAR specifically binds B7H6. Exemplary anti-B7H6 CARs include, without limitation, CYAD-04 (Celyad SA).

In various embodiments, the CAR specifically binds BCMA. Exemplary anti-BCMA CARs include, without limitation, ACTR-087+SEA-BCMA (Seattle Genetics Inc), ALLO-715 (Cellectis SA), ARI-0002 (Institut d'Investigacions Biomediques August Pi I Sunyer), bb-2121 (bluebird bio Inc), bb-21217 (bluebird bio Inc), CART-BCMA (University of Pennsylvania), CT-053 (Carsgen Therapeutics Ltd), Descartes-08 (Cartesian Therapeutics), FCARH-143 (Juno Therapeutics Inc), ICTCAR-032 (Innovative Cellular Therapeutics Co Ltd), IM21 CART (Beijing Immunochina Medical Science & Technology Co Ltd), JCARH-125 (Memorial Sloan-Kettering Cancer Center), KITE-585 (Kite Pharma Inc), LCAR-B38M (Nanjing Legend Biotech Co Ltd), LCAR-B4822M (Nanjing Legend Biotech Co Ltd), MCARH-171 (Memorial Sloan-Kettering Cancer Center), P-BCMA-101 (Poseida Therapeutics Inc), P-BCMA-ALLO1 (Poseida Therapeutics Inc), spCART-269 (Shanghai Unicar-Therapy Bio-medicine Technology Co Ltd), and BCMA02/bb2121 (bluebird bio Inc). The polypeptide sequence of the BCMA02/bb2121 CAR is provided below:

MALPVTALLLPLALLLHAARPDIVLTQSPPSLAMSLGKRATISCRASESV
TILGSHLIHWYQQKPGQPPTLLIQLASNVQTGVPARFSGSGSRTDFTLTI
DPVEEDDVAVYYCLQSRTIPRTFGGGTKLEIKGSTSGSGKPGSGEGSTKG
QIQLVQSGPELKKPGETVKISCKASGYTFTDYSINWVKRAPGKGLKWMGW
INTETREPAYAYDFRGRFAFSLETSASTAYLQINNLKYEDTATYFCALDY
SYAMDYWGQGTSVTVSSAAATTTPAPRPPTPAPTIASQPLSLRPEACRPA
AGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAE
AYSEIGMKGERRRGKGHDGLYQGLSTATKDTYDALHMQALPPR

In various embodiments, the CAR specifically binds CCK2R. Exemplary anti-CCK2R CARs include, without limitation, anti-CCK2R CAR-T adaptor molecule (CAM)+anti-FITC CAR T-cell therapy (cancer), Endocyte/Purdue (Purdue University),

In various embodiments, the CAR specifically binds a CD antigen. Exemplary anti-CD antigen CARs include, without limitation, VM-802 (ViroMed Co Ltd). In various embodiments, the CAR specifically binds CD123. Exemplary anti-CD123 CARs include, without limitation, MB-102 (Fortress Biotech Inc), RNA CART123 (University of Pennsylvania), SFG-iMC-CD123.zeta (Bellicum Pharmaceuticals Inc), and UCART-123 (Cellectis SA). In various embodiments, the CAR specifically binds CD133. Exemplary anti-CD133 CARs include, without limitation, KD-030 (Nanjing Kaedi Biotech Inc). In various embodiments, the CAR specifically binds CD138. Exemplary anti-CD138 CARs include, without limitation, ATLCAR.CD138 (UNC Lineberger Comprehensive Cancer Center) and CART-138 (Chinese PLA General Hospital). In various embodiments, the CAR specifically binds CD171. Exemplary anti-CD171 CARs include, without limitation, JCAR-023 (Juno Therapeutics Inc). In various embodiments, the CAR specifically binds CD19. Exemplary anti-CD19 CARs include, without limitation, 1928z-41BBL (Memorial Sloan-Kettering Cancer Center), 1928z-E27 (Memorial Sloan-Kettering Cancer Center), 19-28z-T2 (Guangzhou Institutes of Biomedicine and Health), 4G7-CARD (University College London), 4SCAR19 (Shenzhen Geno-Immune Medical Institute), ALLO-501 (Pfizer Inc), ATA-190 (QIMR Berghofer Medical Research Institute), AUTO-1 (University College London), AVA-008 (Avacta Ltd), axicabtagene ciloleucel (Kite Pharma Inc), BG-T19 (Guangzhou Bio-gene Technology Co Ltd), BinD-19 (Shenzhen BinDeBio Ltd.), BPX-401 (Bellicum Pharmaceuticals Inc), CAR19h28TM41BBz (Westmead Institute for Medical Research), C-CAR-011 (Chinese PLA General Hospital), CD19CART (Innovative Cellular Therapeutics Co Ltd), CIK-CAR.CD19 (Formula Pharmaceuticals Inc), CLIC-1901 (Ottawa Hospital Research Institute), CSG-CD19 (Carsgen Therapeutics Ltd), CTL-119 (University of Pennsylvania), CTX-101 (CRISPR Therapeutics AG), DSCAR-01 (Shanghai Hrain Biotechnology), ET-190 (Eureka Therapeutics Inc), FT-819 (Memorial Sloan-Kettering Cancer Center), ICAR-19 (Immune Cell Therapy Inc), IM19 CAR-T (Beijing Immunochina Medical Science & Technology Co Ltd), JCAR-014 (Juno Therapeutics Inc), JWCAR-029 (MingJu Therapeutics (Shanghai) Co., Ltd), KD-C-19 (Nanjing Kaedi Biotech Inc), LinCART19 (iCell Gene Therapeutics), lisocabtagene maraleucel (Juno Therapeutics Inc), MatchCART (Shanghai Hrain Biotechnology), MB-CART19.1 (Shanghai Children's Medical Center), PBCAR-0191 (Precision BioSciences Inc), PCAR-019 (PersonGen Biomedicine (Suzhou) Co Ltd), pCAR-19B (Chongqing Precision Biotech Co Ltd), PZ-01 (Pinze Lifetechnology Co Ltd), RB-1916 (Refuge Biotechnologies Inc), SKLB-083019 (Chengdu Yinhe Biomedical Co Ltd), spCART-19 (Shanghai Unicar-Therapy Bio-medicine Technology Co Ltd), TBI-1501 (Takara Bio Inc), TC-110 (TCR2 Therapeutics Inc), TI-1007 (Timmune Biotech Inc), tisagenlecleucel (Abramson Cancer Center of the University of Pennsylvania), U-CART (Shanghai Bioray Laboratory Inc), UCART-19 (Wugen Inc), UCART-19 (Cellectis SA), vadacabtagene leraleucel (Memorial Sloan-Kettering Cancer Center), XLCART-001 (Nanjing Medical University), and yinnuokati-19 (Shenzhen Innovation Immunotechnology Co Ltd). In various embodiments, the CAR specifically binds CD2. Exemplary anti-CD2 CARs include, without limitation, UCART-2 (Wugen Inc). In various embodiments, the CAR specifically binds CD20. Exemplary anti-CD20 CARs include, without limitation, ACTR-087 (National University of Singapore), ACTR-707 (Unum Therapeutics Inc), CBM-C20.1 (Chinese PLA General Hospital), MB-106 (Fred Hutchinson Cancer Research Center), and MB-CART20.1 (Miltenyi Biotec GmbH).

In various embodiments, the CAR specifically binds CD22. Exemplary anti-CD22 CARs include, without limitation, anti-CD22 CAR T-cell therapy (B-cell acute lymphoblastic leukemia), University of Pennsylvania (University of Pennsylvania), CD22-CART (Shanghai Unicar-Therapy Bio-medicine Technology Co Ltd), JCAR-018 (Opus Bio Inc), MendCART (Shanghai Hrain Biotechnology), and UCART-22 (Cellectis SA). In various embodiments, the CAR specifically binds CD30. Exemplary anti-CD30 CARs include, without limitation, ATLCAR.CD30 (UNC Lineberger Comprehensive Cancer Center), CBM-C30.1 (Chinese PLA General Hospital), and Hu30-CD28zeta (National Cancer Institute). In various embodiments, the CAR specifically binds CD33. Exemplary anti-CD33 CARs include, without limitation, anti-CD33 CAR gamma delta T-cell therapy (acute myeloid leukemia), TC BioPharm/University College London (University College London), CAR33VH (Opus Bio Inc), CART-33 (Chinese PLA General Hospital), CIK-CAR.CD33 (Formula Pharmaceuticals Inc), UCART-33 (Cellectis SA), and VOR-33 (Columbia University).

In various embodiments, the CAR specifically binds CD38. Exemplary anti-CD38 CARs include, without limitation, UCART-38 (Cellectis SA). In various embodiments, the CAR specifically binds CD38 A2. Exemplary anti-CD38 A2 CARs include, without limitation, T-007 (TNK Therapeutics Inc). In various embodiments, the CAR specifically binds CD4. Exemplary anti-CD4 CARs include, without limitation, CD4CAR (iCell Gene Therapeutics). In various embodiments, the CAR specifically binds CD44. Exemplary anti-CD44 CARs include, without limitation, CAR-CD44v6 (Istituto Scientifico H San Raffaele). In various embodiments, the CAR specifically binds CD5. Exemplary anti-CD5 CARs include, without limitation, CD5CAR (iCell Gene Therapeutics). In various embodiments, the CAR specifically binds CD7. Exemplary anti-CD7 CARs include, without limitation, CAR-pNK (PersonGen Biomedicine (Suzhou) Co Ltd), and CD7.CAR/28zeta CAR T cells (Baylor College of Medicine), UCART7 (Washington University in St Louis).

In various embodiments, the CAR specifically binds CDH17. Exemplary anti-CDH17 CARs include, without limitation, ARB-001.T (Arbele Ltd). In various embodiments, the CAR specifically binds CEA. Exemplary anti-CEA CARs include, without limitation, HORC-020 (HumOrigin Inc). In various embodiments, the CAR specifically binds Chimeric TGF-beta receptor (CTBR). Exemplary anti-Chimeric TGF-beta receptor (CTBR) CARs include, without limitation, CAR-CTBR T cells (bluebird bio Inc). In various embodiments, the CAR specifically binds Claudin18.2. Exemplary anti-Claudin18.2 CARs include, without limitation, CAR-CLD18 T-cells (Carsgen Therapeutics Ltd) and KD-022 (Nanjing Kaedi Biotech Inc).

In various embodiments, the CAR specifically binds CLL1. Exemplary anti-CLL1 CARs include, without limitation, KITE-796 (Kite Pharma Inc). In various embodiments, the CAR specifically binds DLL3. Exemplary anti-DLL3 CARs include, without limitation, AMG-119 (Amgen Inc). In various embodiments, the CAR specifically binds Dual BCMA/TACI (APRIL). Exemplary anti-Dual BCMA/TACI (APRIL) CARs include, without limitation, AUTO-2 (Autolus Therapeutics Limited). In various embodiments, the CAR specifically binds Dual CD19/CD22. Exemplary anti-Dual CD19/CD22 CARs include, without limitation, AUTO-3 (Autolus Therapeutics Limited) and LCAR-L10D (Nanjing Legend Biotech Co Ltd). In various embodiments, the CAR specifically binds CD19. In various embodiments, the CAR specifically binds Dual CLL1/CD33. Exemplary anti-Dual CLL1/CD33 CARs include, without limitation, ICG-136 (iCell Gene Therapeutics). In various embodiments, the CAR specifically binds Dual EpCAM/CD3. Exemplary anti-Dual EpCAM/CD3 CARs include, without limitation, IKT-701 (Icell Kealex Therapeutics). In various embodiments, the CAR specifically binds Dual ErbB/4ab. Exemplary anti-Dual ErbB/4ab CARs include, without limitation, LEU-001 (King's College London). In various embodiments, the CAR specifically binds Dual FAP/CD3. Exemplary anti-Dual FAP/CD3 CARs include, without limitation, IKT-702 (Icell Kealex Therapeutics). In various embodiments, the CAR specifically binds EBV. Exemplary anti-EBV CARs include, without limitation, TT-18 (Tessa Therapeutics Pte Ltd).

In various embodiments, the CAR specifically binds EGFR. Exemplary anti-EGFR CARs include, without limitation, anti-EGFR CAR T-cell therapy (CBLB MegaTAL, cancer), bluebird bio (bluebird bio Inc), anti-EGFR CAR T-cell therapy expressing CTLA-4 checkpoint inhibitor+PD-1 checkpoint inhibitor mAbs (EGFR-positive advanced solid tumors), Shanghai Cell Therapy Research Institute (Shanghai Cell Therapy Research Institute), CSG-EGFR (Carsgen Therapeutics Ltd), and EGFR-IL12-CART (Pregene (Shenzhen) Biotechnology Co Ltd).

In various embodiments, the CAR specifically binds EGFRvIII. Exemplary anti-EGFRvIII CARs include, without limitation, KD-035 (Nanjing Kaedi Biotech Inc) and UCART-EgfrVIII (Cellectis SA). In various embodiments, the CAR specifically binds Flt3. Exemplary anti-Flt3 CARs include, without limitation, ALLO-819 (Pfizer Inc) and AMG-553 (Amgen Inc). In various embodiments, the CAR specifically binds Folate receptor. Exemplary anti-Folate receptor CARs include, without limitation, EC17/CAR T (Endocyte Inc). In various embodiments, the CAR specifically binds G250. Exemplary anti-G250 CARs include, without limitation, autologous T-lymphocyte cell therapy (G250-scFV-transduced, renal cell carcinoma), Erasmus Medical Center (Daniel den Hoed Cancer Center).

In various embodiments, the CAR specifically binds GD2. Exemplary anti-GD2 CARs include, without limitation, 1RG-CART (University College London), 4SCAR-GD2 (Shenzhen Geno-Immune Medical Institute), C7R-GD2.CART cells (Baylor College of Medicine), CMD-501 (Baylor College of Medicine), CSG-GD2 (Carsgen Therapeutics Ltd), GD2-CARTO1 (Bambino Gesu Hospital and Research Institute), GINAKIT cells (Baylor College of Medicine), iC9-GD2-CAR-IL-15 T-cells (UNC Lineberger Comprehensive Cancer Center), and IKT-703 (Icell Kealex Therapeutics). In various embodiments, the CAR specifically binds GD2 and MUC1. Exemplary anti-GD2/MUC1 CARs include, without limitation, PSMA CAR-T (University of Pennsylvania).

In various embodiments, the CAR specifically binds GPC3. Exemplary anti-GPC3 CARs include, without limitation, ARB-002.T (Arbele Ltd), CSG-GPC3 (Carsgen Therapeutics Ltd), GLYCAR (Baylor College of Medicine), and TT-14 (Tessa Therapeutics Pte Ltd). In various embodiments, the CAR specifically binds Her2. Exemplary anti-Her2 CARs include, without limitation, ACTR-087+trastuzumab (Unum Therapeutics Inc), ACTR-707+trastuzumab (Unum Therapeutics Inc), CIDeCAR (Bellicum Pharmaceuticals Inc), MB-103 (Mustang Bio Inc), RB-H21 (Refuge Biotechnologies Inc), and TT-16 (Baylor College of Medicine). In various embodiments, the CAR specifically binds IL13R. Exemplary anti-IL13R CARs include, without limitation, MB-101 (City of Hope) and YYB-103 (YooYoung Pharmaceuticals Co Ltd). In various embodiments, the CAR specifically binds integrin beta-7. Exemplary anti-integrin beta-7 CARs include, without limitation, MMG49 CAR T-cell therapy (Osaka University). In various embodiments, the CAR specifically binds LC antigen. Exemplary anti-LC antigen CARs include, without limitation, VM-803 (ViroMed Co Ltd) and VM-804 (ViroMed Co Ltd).

In various embodiments, the CAR specifically binds mesothelin. Exemplary anti-mesothelin CARs include, without limitation, CARMA-hMeso (Johns Hopkins University), CSG-MESO (Carsgen Therapeutics Ltd), iCasp9M28z (Memorial Sloan-Kettering Cancer Center), KD-021 (Nanjing Kaedi Biotech Inc), m-28z-T2 (Guangzhou Institutes of Biomedicine and Health), MesoCART (University of Pennsylvania), meso-CAR-T+PD-78 (MirImmune LLC), RB-M1 (Refuge Biotechnologies Inc), and TC-210 (TCR2 Therapeutics Inc).

In various embodiments, the CAR specifically binds MUC1. Exemplary anti-MUC1 CARs include, without limitation, anti-MUC1 CAR T-cell therapy+PD-1 knockout T cell therapy (esophageal cancer/NSCLC), Guangzhou Anjie Biomedical Technology/University of Technology Sydney (Guangzhou Anjie Biomedical Technology Co LTD), ICTCAR-043 (Innovative Cellular Therapeutics Co Ltd), ICTCAR-046 (Innovative Cellular Therapeutics Co Ltd), P-MUCIC-101 (Poseida Therapeutics Inc), and TAB-28z (OncoTab Inc). In various embodiments, the CAR specifically binds MUC16. Exemplary anti-MUC16 CARs include, without limitation, 4H1128Z-E27 (Eureka Therapeutics Inc) and JCAR-020 (Memorial Sloan-Kettering Cancer Center).

In various embodiments, the CAR specifically binds nfP2X7. Exemplary anti-nfP2X7 CARs include, without limitation, BIL-022c (Biosceptre International Ltd). In various embodiments, the CAR specifically binds PSCA. Exemplary anti-PSCA CARs include, without limitation, BPX-601 (Bellicum Pharmaceuticals Inc). In various embodiments, the CAR specifically binds PSMA. CIK-CAR.PSMA (Formula Pharmaceuticals Inc), and P-PSMA-101 (Poseida Therapeutics Inc). In various embodiments, the CAR specifically binds ROR1. Exemplary anti-ROR1 CARs include, without limitation, JCAR-024 (Fred Hutchinson Cancer Research Center). In various embodiments, the CAR specifically binds ROR2. Exemplary anti-ROR2 CARs include, without limitation, CCT-301-59 (F1 Oncology Inc). In various embodiments, the CAR specifically binds SLAMF7. Exemplary anti-SLAMF7 CARs include, without limitation, UCART-CS1 (Cellectis SA). In various embodiments, the CAR specifically binds TRBC1. Exemplary anti-TRBC1 CARs include, without limitation, AUTO-4 (Autolus Therapeutics Limited). In various embodiments, the CAR specifically binds TRBC2. Exemplary anti-TRBC2 CARs include, without limitation, AUTO-5 (Autolus Therapeutics Limited). In various embodiments, the CAR specifically binds TSHR. Exemplary anti-TSHR CARs include, without limitation, ICTCAT-023 (Innovative Cellular Therapeutics Co Ltd). In various embodiments, the CAR specifically binds VEGFR-1. Exemplary anti-VEGFR-1 CARs include, without limitation, SKLB-083017 (Sichuan University).

In various embodiments, the CAR is AT-101 (AbClon Inc); AU-101, AU-105, and AU-180 (Aurora Biopharma Inc); CARMA-0508 (Carisma Therapeutics); CAR-T (Fate Therapeutics Inc); CAR-T (Cell Design Labs Inc); CM-CX1 (Celdara Medical LLC); CMD-502, CMD-503, and CMD-504 (Baylor College of Medicine); CSG-002 and CSG-005 (Carsgen Therapeutics Ltd); ET-1501, ET-1502, and ET-1504 (Eureka Therapeutics Inc); FT-61314 (Fate Therapeutics Inc); GB-7001 (Shanghai GeneChem Co Ltd); IMA-201 (Immatics Biotechnologies GmbH); IMM-005 and IMM-039 (Immunome Inc); ImmuniCAR (TC BioPharm Ltd); NT-0004 and NT-0009 (BioNTech Cell and Gene Therapies GmbH), OGD-203 (OGD2 Pharma SAS), PMC-005B (PharmAbcine), and TI-7007 (Timmune Biotech Inc).

In some embodiments the chimeric antigen receptor comprises an amino acid sequence of an antibody. In some embodiments, the chimeric antigen receptor comprises the amino acid sequence of an antigen binding fragment of an antibody. The antibody (or fragment thereof) portion of the extracellular binding domain recognizes and binds to an epitope of an antigen. In some embodiments, the antibody fragment portion of a chimeric antigen receptor is a single chain variable fragment (scFv). An scFV comprises the light and variable fragments of a monoclonal antibody. In other embodiments, the antibody fragment portion of a chimeric antigen receptor is a multichain variable fragment, which can comprise more than one extracellular binding domains and therefore bind to more than one antigen simultaneously. In a multiple chain variable fragment embodiment, a hinge region may separate the different variable fragments, providing necessary spatial arrangement and flexibility.

In other embodiments, the antibody portion of a chimeric antigen receptor comprises at least one heavy chain and at least one light chain. In some embodiments, the antibody portion of a chimeric antigen receptor comprises two heavy chains, joined by disulfide bridges and two light chains, wherein the light chains are each joined to one of the heavy chains by disulfide bridges. In some embodiments, the light chain comprises a constant region and a variable region. Complementarity determining regions residing in the variable region of an antibody are responsible for the antibody's affinity for a particular antigen. Thus, antibodies that recognize different antigens comprise different complementarity determining regions. Complementarity determining regions reside in the variable domains of the extracellular binding domain, and variable domains (i.e., the variable heavy and variable light) can be linked with a linker or, in some embodiments, with disulfide bridges.

In some embodiments, the antigen recognized and bound by the extracellular domain is a protein or peptide, a nucleic acid, a lipid, or a polysaccharide. Antigens can be heterologous, such as those expressed in a pathogenic bacteria or virus. Antigens can also be synthetic; for example, some individuals have extreme allergies to synthetic latex and exposure to this antigen can result in an extreme immune reaction. In some embodiments, the antigen is autologous, and is expressed on a diseased or otherwise altered cell. For example, in some embodiments, the antigen is expressed in a neoplastic cell. In some embodiments, the neoplastic cell is a solid tumor cell. In other embodiments, the neoplastic cell is a hematological cancer, such as a B cell cancer. In some embodiments, the B cell cancer is a lymphoma (e.g., Hodgkins or non-Hodgkins lymphoma) or a leukemia (e.g., B-cell acute lymphoblastic leukemia). Exemplary B-cell lymphomas include Diffuse large B-cell lymphoma (DLBCL), primary mediastinal B-cell lymphoma, follicular lymphoma, Chronic lymphocytic leukemia (CLL), small lymphocytic lymphoma (SLL), mantle cell lymphomas, Marginal zone lymphoma, Burkitt lymphoma, Burkitt-like lymphoma, Lymphoplasmacytic lymphoma (Waldenstrom macroglobulinemia), and hairy cell leukemia. In some embodiments, the B cell cancer is multiple myeloma.

Antibody-antigen interactions are noncovalent interactions resulting from hydrogen bonding, electrostatic or hydrophobic interactions, or from van der Waals forces. The affinity of extracellular binding domain of the chimeric antigen receptor for an antigen can be calculated with the following formula:

K_{A}=[Antibody−Antigen]/[Antibody][Antigen], wherein

[Ab]=molar concentration of unoccupied binding sites on the antibody;
[Ag]=molar concentration of unoccupied binding sites on the antigen; and
[Ab-Ag]=molar concentration of the antibody-antigen complex.

The antibody-antigen interaction can also be characterized based on the dissociation of the antigen from the antibody. The dissociation constant (K_{D}) is the ratio of the association rate to the dissociation rate and is inversely proportional to the affinity constant. Thus, K_{D}=1/K_{A}. Those skilled in the art will be familiar with these concepts and will know that traditional methods, such as ELISA assays, can be used to calculate these constants.

The transmembrane domain of the chimeric antigen receptors described herein spans the CAR-T cells lipid bilayer cellular membrane and separates the extracellular binding domain and the intracellular signaling domain. In some embodiments, this domain is derived from other receptors having a transmembrane domain, while in other embodiments, this domain is synthetic. In some embodiments, the transmembrane domain may be derived from a non-human transmembrane domain and, in some embodiments, humanized. By “humanized” is meant having the sequence of the nucleic acid encoding the transmembrane domain optimized such that it is more reliably or efficiently expressed in a human subject. In some embodiments, the transmembrane domain is derived from another transmembrane protein expressed in a human immune effector cell. Examples of such proteins include, but are not limited to, subunits of the T cell receptor (TCR) complex, PD1, or any of the Cluster of Differentiation proteins, or other proteins, that are expressed in the immune effector cell and that have a transmembrane domain. In some embodiments, the transmembrane domain will be synthetic, and such sequences will comprise many hydrophobic residues.

The chimeric antigen receptor is designed, in some embodiments, to comprise a spacer between the transmembrane domain and the extracellular domain, the intracellular domain, or both. Such spacers can be 1, 2, 3, 4, 5, 6, 7, 8, 9, 10, 11, 12, 13, 14, 15, 16, 17, 18, 19, or 20 amino acids in length. In some embodiments, the spacer can be 20, 30, 40, 50, 60, 70, 80, 90, or 100 amino acids in length. In still other embodiments the spacer can be between 100 and 500 amino acids in length. The spacer can be any polypeptide that links one domain to another and are used to position such linked domains to enhance or optimize chimeric antigen receptor function.

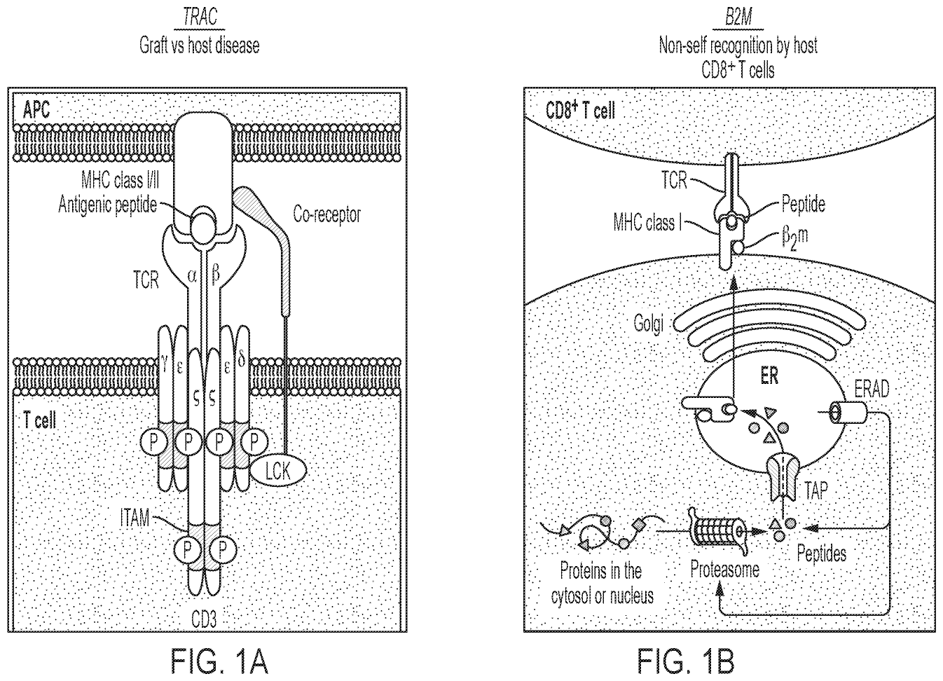

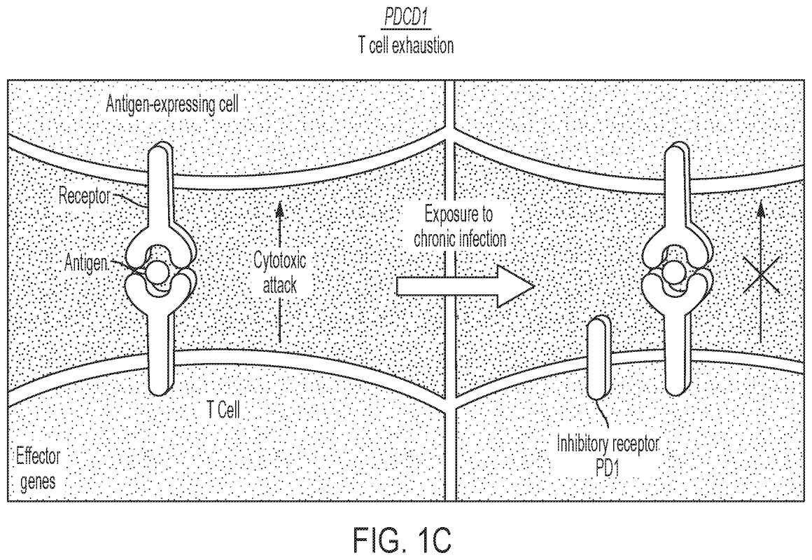

The intracellular signaling domain of the chimeric antigen receptor contemplated herein comprises a primary signaling domain. In some embodiments, the chimeric antigen receptor comprises the primary signaling domain and a secondary, or co-stimulatory, signaling domain. In some embodiments, the primary signaling domain comprises one or more immunoreceptor tyrosine-based activation motifs, or ITAMs. In some embodiments, the primary signaling domain comprises more than one ITAM. ITAMs incorporated into the chimeric antigen receptor may be derived from ITAMs from other cellular receptors. In some embodiments, the primary signaling domain comprising an ITAM may be derived from subunits of the TCR complex, such as CD3γ, CD3ε, CD3ζ, or CD3δ (see FIG. 1A). In some embodiments, the primary signaling domain comprising an ITAM may be derived from FcRγ, FcRβ, CD5, CD22, CD79a, CD79b, or CD66d. The secondary signaling domain, in some embodiments, is derived from CD28. In other embodiments, the secondary signaling domain is derived from CD2, CD4, CDS, CD8a, CD83, CD134, CD137, ICOS, or CD154.

Provided herein are also nucleic acids that encode the chimeric antigen receptors described herein. In some embodiments, the nucleic acid is isolated or purified. Delivery of the nucleic acids ex vivo can be accomplished using methods known in the art. For example, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

Some aspects of the present invention provide for immune cells comprising a chimeric antigen and an altered endogenous gene that enhances immune cell function, resistance to immunosuppression or inhibition, or a combination thereof. In some embodiments, the altered endogenous gene may be created by base editing. In some embodiments, the base editing may reduce or attenuate the gene expression. In some embodiments, the base editing may reduce or attenuate the gene activation. In some embodiments, the base editing may reduce or attenuate the functionality of the gene product. In some other embodiments, the base editing may activate or enhance the gene expression. In some embodiments, the base editing may increase the functionality of the gene product. In some embodiments, the altered endogenous gene may be modified or edited in an exon, an intron, an exon-intron injunction, or a regulatory element thereof. The modification may be edit to a single nucleobase in a gene or a regulatory element thereof. The modification may be in a exon, more than one exons, an intron, or more than one introns, or a combination thereof. The modification may be in an open reading frame of a gene. The modification may be in an untranslated region of the gene, for example, a 3′-UTR or a 5′-UTR. In some embodiments, the modification is in a regulatory element of an endogenous gene. In some embodiments, the modification is in a promoter, an enhancer, an operator, a silencer, an insulator, a terminator, a transcription initiation sequence, a translation initiation sequence (e.g. a Kozak sequence), or any combination thereof.

Allogeneic immune cells expressing an endogenous immune cell receptor as well as a chimeric antigen receptor may recognize and attack host cells, a circumstance termed graft versus host disease (GVHD). The alpha component of the immune cell receptor complex is encoded by the TRAC gene, and in some embodiments, this gene is edited such that the alpha subunit of the TCR complex is nonfunctional or absent. Because this subunit is necessary for endogenous immune cell signaling, editing this gene can reduce the risk of graft versus host disease caused by allogeneic immune cells.

Host immune cells can potentially recognize allogeneic CAR-T cells as non-self and elicit an immune response to remove the non-self cells. B2M is expressed in nearly all nucleated cells and is associated with MHC class I complex (FIG. 1B). Circulating host CD8⁺ T cells can recognize this B2M protein as non-self and kill the allogeneic cells. To overcome this graft rejection, in some embodiments, the B2M gene is edited to either knockout or knockdown expression.

In some embodiments of the present invention, the PDCD1 gene is edited in the CAR-T cell to knockout or knockdown expression. The PDCD1 gene encodes the cell surface receptor PD-1, an immune system checkpoint expressed in immune cells, and it is involved in reducing autoimmunity by promoting apoptosis of antigen specific immune cells. By knocking out or knocking down expression of the PDCD1 gene, the modified CAR-T cells are less likely to apoptose, are more likely to proliferate, and can escape the programmed cell death immune checkpoint.

The CBLB gene encodes an E3 ubiquitin ligase that plays a significant role in inhibiting immune effector cell activation. Referring to FIG. 1C, the CBLB protein favors the signaling pathway resulting in immune effector cell tolerance and actively inhibits signaling that leads to immune effector cell activation. Because immune effector cell activation is necessary for the CAR-T cells to proliferate in vivo post-transplant, in some embodiments of the present invention the CBLB is edited to knockout or knockdown expression.

In some embodiments, editing of genes to enhance the function of the immune cell or to reduce immunosuppression or inhibition can occur in the immune cell before the cell is transformed to express a chimeric antigen receptor. In other aspects, editing of genes to enhance the function of the immune cell or to reduce immunosuppression or inhibition can occur in a CAR-T cell, i.e., after the immune cell has been transformed to express a chimeric antigen receptor.

In some embodiments, the immune cell may comprise a chimeric antigen receptor (CAR) and one or more edited genes, one or more regulatory elements thereof, or combinations thereof, wherein expression of the edited gene is either knocked out or knocked down. In some embodiments, the CAR-T cells have reduced immunogenicity as compared to a similar CAR-T cell but without further having the one or more edited genes as described herein. In some embodiments, the CAR-T cells have lower activation threshold as compared to a similar CAR-T but without further having the one or more edited genes as described herein. In some embodiments, the CAR-T cells have increased anti-neoplasia activity as compared to a similar CAR-T cell but without further having the one or more edited genes as described herein. The one or more genes may be edited by base editing. In some embodiments the one or more genes, or one or more regulatory elements thereof, or combinations thereof, may be selected from a group consisting of: c-abl oncogene 1 (Abl1); c-abl oncogene 2 (Abl2); a disintegrin and metalloprotease domain 8 (Adam8); a disintegrin and metalloprotease domain 17 (Adam 17); adenosine deaminase (Ada); adenosine kinase (Adk); adenosine A2a receptor (Adora2a); adenosine regulating molecule 1 (Adrm1); advanced glycosylation end product-specific receptor (Ager) allograft inflammatory factor 1 (Aif1); autoimmune regulator (Aire); ankyrin repeat and LEM domain (Ankle1); annecin A1 (Anxa1); adapter related protein complex 3 beta 1 sububit (Ap3b1); adapter related protein complex 3 delta 1 sububit (Ap3d1); amyloid beta (A4) precursor protein-binding family B member 1 interacting protein (Apbb1ip); WNT signaling pathway regulator (Apc); arginase liver (Arg 1); arginase type II (Arg 2); autophagy related 5 (Atg5); AtPase Cu++ transporting, alpha polypeptide (Atp7a); 5-azacytidine induced gene 2 (Azi2); beta 2 microglobulin (B2m); BL2-associated agonist of cell dealth (Bad); basic leucine zipper transcription factor, ATF-like (Batf); BCL2-associated X protein (Bax); B cell leukemia/lymphoma 2 (Bcl2); B cell leukemia/lymphoma 2 related protein A1d (Bcl2a1d); B cell leukemia/lymphoma 3 (Bcl3); B cell leukemia/lymphoma 6 (Bcl6); B cell leukemia/lymphoma 10 (Bcl10); B cell leukemia/lymphoma 11a (Bcllla); B cell leukemia/lymphoma 11b (Bcl11b); Bloom syndrome, RecQ like helicase (Blm); Bmi1 polycomb ring finger oncogene (Bmi1); Bone morphogenic protein 4 (Bmp4); Braf transforming gene (Braf); B and T lymphocyte associated (Btla); butyrophilin, subfamily 2, member A1 (Btn2a1); butyrophilin, subfamily 2, member A2 (Btn2a2); butyrophilin-like 1 (Btnl1); butyrophilin-like 2 (Btnl2); butyrophilin-like 6 (Btnl6); calcium channel, voltage dependent, beta 4 subunit (Cacnb4); caspase recruitment domain family member 11 (Card 11); capping protein regulator and myosin 1 linker 2 (Carmil2); Caspase 3 (Casp3); caveolin 1 (Cav1); core-binding factor beta (Cbfb); Casitas B-lineage lymphoma b (Cblb); coil-coil domain containing 88B (Ccdc88b); chemokine (C—C motif) ligand 2 (Ccl2); chemokine (C—C motif) ligand 5 (Ccl5); chemokine (C—C motif) ligand 19 (Ccl19); chemokine (C—C motif) ligand 20 (Ccl20); cyclin D3 (Ccnd3); chemokine (C—C motif) receptor 2 (Ccr2); chemokine (C—C motif) receptor 6 (Ccr6); chemokine (C—C motif) receptor 7 (Ccr7); chemokine (C—C motif) receptor 9 (Ccr9); CD1d1 antigen (Cd1d1); CD1d2 antigen (CD1d2); CD2 antigen (CD2); CD3 antigen, delta polypeptide (CD3d); CD3 antigen, epsilon polypeptide (CD3d); CD4 antigen (Cd4); CD5 antigen (Cd5); CD6 antigen (Cd6); CD8 antigen (Cd8); CD24a antigen (Cd24a); CD27 antigen (CD27); CD28 antigen (Cd28); CD40 ligand (Cd401g); CD44 antigen (Cd44); CD46 antigen, complement regulatory protein (Cd46); CD47 antigen (Rh-related antigen, integrin-associated signal transducer) (Cd47); CD48 antigen (Cd48); CD59b antigen (Cd59b); CD74 antigen (Cd74); CD80 antigen (Cd80); CD81 antigen (Cd81); CD83 antigen (Cd83); CD86 antigen (Cd86); CD151 antigen (Cd151); CD160 antigen (Cd160); CD209e antigen (Cd209e); CD244 molecule A (Cd244a); CD274 antigen (Cd274); CD276 antigen (Cd276); CD300A molecule (Cd300a); cadherin-like 26(Cdh26); cyclin-dependent kinase (Cdk6); cyclin dependent kinase inhibitor 2A (Cdkn2a); carcinoembryonic antigen-related cell adhesion molecule (Ceacam1); CCAAT/enhancer binding protein (C/EBP), beta (Cebpb); cyclic GMP-AMP synthase (Cgas); chromodomain helicase DNA binding protein 7 (Chd7); cholinergic receptor, nicotinic, alpha polypeptide 7 (Chrna7); C-type lectin domain family 2, member i (Clec2i); C-type lectin domain family 4, member a2 (Clec4a2); C-type lectin domain family 4, member d (Clec4d); C-type lectin domain family 4, member e (Clec4e); C-type lectin domain family 4, member f (Clec4f); C-type lectin domain family 4, member g (Clec4g); cleft lip and palate associated transmembrane protein 1 (Clptm1); coronin, actin binding protein 1A (Coro1a); cysteine-rich protein 3 (Crip3); c-src tyrosine kinase (Csk); cytotoxic T lymphocyte-associated protein 2 alpha (Ctla2a); cytotoxic T-lymphocyte-associated protein 4 (Ctla4); catenin (cadherin associated protein), beta 1 (Ctnnb1); cytidine 5′-triphosphate synthase (Ctps); coxsackie virus and adenovirus receptor (Cxadr); chemokine (C—X—C motif) ligand 12 (Cxcl12); chemokine (C—X—C motif) receptor (Cxcr4); CYLD lysine 63 deubiquitinase (Cyld); cytochrome P450, family 26, subfamily b, polypeptide (Cyp26b1); dolichyl-di-phosphooligosaccharide-protein glycotransferase (Ddost); deoxyhypusine synthase (Dhps); dicer 1, ribonuclease type III (Dicer1); discs large MAGUK scaffold protein 1 (Dlg1); discs large MAGUK scaffold protein 5 (Dlg5); delta like canonical Notch ligand 4 (D114); DnaJ heat shock protein family (Hsp40) member A3 (Dnaja3); dedicator of cytokinesis 2 (Dock2); dedicator of cytokinesis 8 (Dock8); dipeptidylpeptidase 4 (Dpp4); drosha, ribonuclease type III (Drosha); deltex 1, E3 ubiquitin ligase (Dtx1); dual specificity phosphatase 3 (Dusp3); dual specificity phosphatase 10 (Dusp10); dual specificity phosphatase 22 (Dusp22); double homeobox B-like 1 (Duxb11); Epstein-Barr virus induced gene 3 (Ebi3); ephrin B1 (Efnb1); ephrin B2 (Efnb2); ephrin B3 (Efnb3); early growth response 1(Egr1); early growth response 3 (Egr3); eukaryotic translation initiation factor 2 alpha kinase 4 (Eif2ak4); E74-like factor 4 (Elf4); eomesodermin (Eomes); Eph receptor B4 (Ephb4); Eph receptor B6 (Ephb6); erythropoietin (Epo); erb-b2 receptor tyrosine kinase (Erbb2); coagulation factor II (thrombin) receptor-like 1 (F2rl1); Fas (TNFRSF6)-associated via death domain (Fadd); family with sequence similarity 49, member B (Fam49b); Fanconi anemia, complementation group A (Fanca); Fanconi anemia, complementation group D2 (Fancd2); Fas (TNF receptor superfamily member 6) (Fas); Fc receptor, IgE, high affinity I, gamma polypeptide (Fcerlg); fibrinogen-like protein 1 (Fgl1); fibrinogen-like protein 2 (Fgl2); FK506 binding protein 1a (Fkbp1a); FK506 binding protein 1b ((Fkbp1b); flotillin 2 (Flot2); FMS-like tyrosine kinase (Flt3); forkhead box J1 (Foxj1); forkhead box N1 (Foxn1); forkhead box P1 (Foxp1); forkhead box P3 (Foxp3); fucosyltransferase 7 (Fut7); Fyn proto-oncogene (Fyn); frizzled class receptor 5 (Fzd5); frizzled class receptor 7 (Fzd7); frizzled class receptor 8 (Fzd8); growth arrest and DNA-damage-inducible 45 gamma (Gadd45g); GATA binding protein 3 (GATA3); GTPase, IMAP family member 1 (Gimap1); gap junction protein, alpha 1 (Gja1); GLI-Kruppel family member GLI3 (Gli3); glycerol-3-phosphate acyltransferase, mitochondrial (Gpam); G protein-coupled receptor 18 (Gpr18); gelsolin (Gsn); histocompatibility 2, class II antigen A, alpha (H2-Aa); histocompatibility 2, class II antigen A, beta 1 (H2-Ab1); histocompatibility 2, class II, locus DMa (H2-DMa); histocompatibility 2, M region locus 3(H3-M3); histocompatibility 2, O region alpha locus (H2-Oa); histocompatibility 2, T region locus 23 (H2-T23); hepatitis A virus cellular receptor 2 (Havcr2); haematopoietic 1 (hem1); hes family bHLH transcription factor 1 (Hes1); homeostatic iron regulator (Hfe); H2.0-like homeobox (Hlx); HCLS1 binding protein 3 (Hslbp3); hematopoietic SH2 domain containing (Hsh2d); heat shock protein 90, alpha (cytosolic), class A member 1 (Hsp90aa1); heat shock protein 1 (chaperonin) (Hspd1); heat shock 105 kDa/110 kDa protein 1 (Hsph1); intercellular adhesion molecule 1 (Icam1); inducible T cell co-stimulator (Icos); icos ligand (Icos1); indoleamine 2,3-dioxygenase 1 (Ido1); interferon alpha 1 (Ifna1); interferon alpha 2 (Ifna2); interferon alpha 4 (Ifna4); interferon alpha 5 (Ifna5); interferon alpha 6 (Ifna6); interferon alpha 7 (Ifna7); interferon alpha 9 (Ifna9); interferon alpha 11 (Ifna11); interferon alpha 12 (Ifna12); interferon alpha 13 (Ifna13); interferon alpha 14 (Ifna14); interferon alpha 15 (Ifna15); interferon alpha 16 (Ifna16); interferon alpha B (Ifnab); interferon (alpha and beta) receptor 1 (Ifnar1); interferon beta 1 (Ifnb1); interferon gamma (Ifng); interferon kappa (Ifnk); interferon zeta (Ifnz); insulin-like growth factor 1 (Igf1); insulin-like growth factor 2 (Igf2); insulin-like growth factor binding protein 2 (Igfbp2); Indian hedgehog (Ihh); IKAROS family zinc finger 1 (Ikzf1); interleukin 1 beta (Il1b; interleukin 1 family, member 8 (Il1f); interleukin 1 receptor-like 2 (Il1r12); interleukin 2 (Il2); interleukin 2 receptor, alpha chain (Il2ra); interleukin 2 receptor, gamma chain (Il2rg); interleukin 4 (Il4); interleukin 4 receptor, alpha (Il4ra); interleukin 6 (Il6); interleukin 6 signal transducer (Il6st); interleukin 7 (Il7); interleukin 7 receptor (Il7r); interleukin 12a (Il12a); interleukin 12b (Il12b); interleukin 12 receptor, beta1 (Il12rb1); interleukin 15 (Il15); interleukin 18 (Il18); interleukin 18 receptor 1 (Il18r1); interleukin 20 receptor beta (Il20rb); interleukin 21 (Il21); interleukin 23, alpha subunit p19 (1123a); interleukin 27 (Il27); insulin II (Ins2); interferon regulatory factor 1 (Irf1); interferon regulatory factor 4 (Irf4); itchy, E3 ubiquitin protein ligase (Itch); integrin, alpha D (Itgad); integrin alpha L (Itga1); integrin alpha M (Itgam); integrin alpha V (Itgav); integrin alpha X (Itgax); integrin beta 2 (Itgb2); IL2 inducible T cell kinase (Itk); inositol 1,4,5-trisphosphate 3-kinase B (Itpkb); jagged 2 (Jag2); Janus kinase 3 (Jak3); junction adhesion molecule like 9 (Jam9); jumonji domain containing 6 (Jmjd6); K (lysine) acetyltransferase 2A (Kat2a); KDEL (Lys-Asp-Glu-Leu) endoplasmic reticulum protein retention receptor 1 (Kdelr1); KIT proto-oncogene receptor tyrosine kinase (Kit); lymphocyte-activation gene 3 (Lag3); linker for activation of T cells (Lat); lymphocyte transmembrane adaptor 1 (Lax1); lymphocyte protein tyrosine kinase (Lck); lymphocyte cytosolic protein 1 (Lcp1); lymphoid enhancer binding factor 1 (Lef1); leptin (Lep); leptin receptor (Lepr); LFNG O-fucosylpeptide 3-beta-N-acetylglucosaminyltransferase (Lfng); lectin, galactose binding, soluble 1 (Lgals1); lectin, galactose binding, soluble 3 (Lgals3); lectin, galactose binding, soluble 8 (Lgals8); lectin, galactose binding, soluble 9 (Lgals9); ligase IV, DNA, ATP-dependent (Lig4); leukocyte immunoglobulin-like receptor, subfamily B, member 4A (Lilrb4a); limb region 1 like (Lmbr1); LIM domain only 1 (Lmo1); lysyl oxidase-like 3 (Loxl3); leucine rich repeat containing 32 (Lrrc32); lymphocyte antigen 9 (Ly9); MAD1 mitotic arrest deficient 1-like 1 (Mad1l1); v-maf musculoaponeurotic fibrosarcoma oncogene family, protein B (avian) (Mafb); MALT1 paracaspase (Malt1); mitogen-activated protein kinase 8 interacting protein 1 (Mapk8ip10); membrane associated ring-CH-type finger 7 (Marchf7); midkine (Mdk); methyltransferase like 3 (Mettl3); MHC I like leukocyte 2 (Mill2); myelin protein zero-like 2 (Mpzl2); moesin (Msn); mechanistic target of rapamycin kinase (Mtor); myeloblastosis oncogene (Myb); myosin, heavy polypeptide 9, non-muscle (Myh9); non-SMC condensin II complex, subunit H2 (Ncaph2); non-catalytic region of tyrosine kinase adaptor protein 1 (Nck1); non-catalytic region of tyrosine kinase adaptor protein 2 (Nck2); NCK associated protein 1 like (Nckap1l); nuclear receptor co-repressor 1 (Ncor1); nicastrin (Ncstn); Nedd4 family interacting protein 1 (Ndfip1); neural precursor cell expressed, developmentally down-regulated 4 (Nedd4); nuclear factor of activated T cells, cytoplasmic, calcineurin dependent (Nfatc3); nuclear factor of kappa light polypeptide gene enhancer in B cells inhibitor, delta (Nfkbid); non-homologous end joining factor 1 (Nhej1); NFKB activating protein (Nkap); NK2 homeobox 3 (Nkx2-3); NLR family, CARD domain containing 3 (Nlrc3); NLR family, pyrin domain containing 3 (Nlrp3); Notch-regulated ankyrin repeat protein (Nrarp); OTU domain containing 5 (Otud5); purinergic receptor P2X, ligand-gated ion channel, 7 (P2rx7); phosphoprotein associated with glycosphingolipid microdomains 1 (Pag1); POZ (BTB) and AT hook containing zinc finger 1 (Patz1); PRKC, apoptosis, WT1, regulator (Pawr); paired box 1 (Pax1); programmed cell death 1 ligand 2 (Pdcd1lg2); phosphodiesterase 5A, cGMP-specific (Pde5a); pellino 1 (Peli1); phosphoinositide-3-kinase regulatory subunit (Pik3r6); phospholipase A2, group IIA (Pla2g2a); phospholipase A2, group IID (Pla2g2d); phospholipase A2, group IIE (Pla2g2e); phospholipase A2, group IIF (Pla2g2f); purine-nucleoside phosphorylase (Pnp); protein phosphatase 3, catalytic subunit, beta isoform (Ppp3cb); PR domain containing 1, with ZNF domain (Prdm1); peroxiredoxin 2 (Prdx2); protein kinase, cAMP dependent regulatory, type I, alpha (Prkar1a); protein kinase C, theta 2 (Prkcq); protein kinase C, zeta (Prkcz); protein kinase, DNA activated, catalytic polypeptide (Prkdc); prosaposin (Psap); presenilin 1 (Psen1); presenilin 2 (Psen2); prostaglandin E receptor 4 (subtype EP4) (Ptger4); protein tyrosine phosphatase, non-receptor type 2 (Ptpn2); protein tyrosine phosphatase, non-receptor type 6 (Ptpn6); protein tyrosine phosphatase, non-receptor type 22 (lymphoid) (Ptpn22); protein tyrosine phosphatase, receptor type, C (Ptprc); PYD and CARD domain containing 7 (Pycard); RAB27A, member RAS oncogene family (Rab27a); RAB29, member RAS oncogene family (Rab29); (Rac family small GTPase 2); recombination activating gene 1 (Rag1); recombination activating gene 2 (Rag2); RAS protein activator like 3 (Rasal3); RAS guanyl releasing protein 1 (Rasgrp1); RING CCCH (C3H) domains 1 (Rc3h1); ring finger and CCCH-type zinc finger domains 2 (Rc3h2); ras homolog family member A (Rhoa); ras homolog family member H (Rhoh); receptor (TNFRSF)-interacting serine-threonine kinase 2 (Ripk2); RHO family interacting cell polarization regulator 2 (Ripor2); RAR-related orphan receptor alpha (Rora); RAR-related orphan receptor gamma (Ror); ribosomal protein L22 (Rpl 22); ribosomal protein S6 (Rps6); radical S-adenosyl methionine domain containing 2 (Rsad2); runt related transcription factor 1 (Runx1); runt related transcription factor 2 (Runx2); runt related transcription factor 3 (Runx3); squamous cell carcinoma antigen recognized by T cells (Sart1); SAM and SH3 domain containing 3 (Sash3); special AT-rich sequence binding protein 1 (Satb1); syndecan 4 (Sdc4); selenoprotein K (Selenok); sema domain, immunoglobulin domain (Ig), transmembrane domain (TM) and short cytoplasmic domain, (semaphorin) 4A (Sema4a); surfactant associated protein D (Sftpd); SH3 domain containing ring finger 1 (Sh3rf1); src homology 2 domain-containing transforming protein B (Shb); sonic hedgehog (Shh); signal-regulatory protein alpha (Sirpa); Signal-regulatory protein beta 1A (Sirpb1a); Signal-regulatory protein beta 1B (Sirpb1b); Signal-regulatory protein beta 1C (Sirpb1c); suppression inducing transmembrane adaptor 1 (Sit1); Src-like-adaptor 2 (Sla2); SLAM family member 6 (Slamf6); solute carrier family 4 (anion exchanger), member 1; (Slc4a1); solute carrier family 11 (proton-coupled divalent metal ion transporters), member 1 (Slc11a1); solute carrier family 46, member 2 (Slc46a2); schlafen 1; SMAD family member 3 (Smad3); SMAD family member 7 (Smad7); suppressor of cytokine signaling 1 (Socs1); suppressor of cytokine signaling 5 (Socs5); suppressor of cytokine signaling 6 (Socs6); SOS Ras/Rac guanine nucleotide exchange factor 1 (Sos1), SOS Ras/Rac guanine nucleotide exchange factor 2 (Sos2), SRY (sex determining region Y)-box 4 (Sox4); sialophorin (Spn); signal transducer and activator of transcription 3 (Stat3); signal transducer and activator of transcription 5A (Stat5A); signal transducer and activator of transcription 5B (Stat5B); serine/threonine kinase 11 (Stk11); syntaxin 11 (Stx11); spleen tyrosine kinase (Syk); T cell-interacting, activating receptor on myeloid cells 1 (Tarm1); T-box 21 (Tbx21); T cell, immune regulator 1, ATPase, H+ transporting, lysosomal VO protein A3 (Tcirg1); transforming growth factor, beta 1 (Tgfb1); transforming growth factor, beta receptor II (Tgfbr2); thymocyte selection associated (Themis); thymus cell antigen 1, theta (Thy1); T cell immunoreceptor with Ig and ITIM domains (Tigit); transmembrane protein 98 (Tmem98); transmembrane 131 like (Tmem131l); tumor necrosis factor, alpha-induced protein 8-like 2 (Tnfalp8l2); tumor necrosis factor receptor superfamily, member 4 (Tnfrsf4); tumor necrosis factor receptor superfamily, member 13c (Tnfrsf13c); tumor necrosis factor (ligand) superfamily, member 4 (Tnfsf4); tumor necrosis factor (ligand) superfamily, member 8 (Tnfsf8); tumor necrosis factor (ligand) superfamily, member 9 (Tnfsf9); tumor necrosis factor (ligand) superfamily, member 11 (Tnfsf11); tumor necrosis factor (ligand) superfamily, member 13b (Tnfsf13b); tumor necrosis factor (ligand) superfamily, member 14 (Tnfsf14); tumor necrosis factor (ligand) superfamily, member 18 (Tnfsf18); TNF receptor-associated factor 6 (Traf6); triggering receptor expressed on myeloid cells-like 2 (Trem12); T cell receptor alpha joining 18 (Traj18); three prime repair exonuclease 1 (Trex1); transformation related protein 53 (Trp53); TSC complex subunit 1 (Tsc1); twisted gastrulation BMP signaling modulator 1 (Twsg1); vascular cell adhesion molecule 1 (Vcam1); vanin 1 (Vnn1); V-set and immunoglobulin domain containing 4 (Vsig4); WD repeat and FYVE domain containing 4 (Wdfy4); wingless-type MMTV integration site family, member 1 (Wnt1); wingless-type MMTV integration site family, member 4 (Wnt4); WW domain containing E3 ubiquitin protein ligase 1 (Wwp1); chemokine (C motif) ligand 1 (Xcl1); zinc finger and BTB domain containing 1 (Zbtb1); zinc finger and BTB domain containing 7B (Zbtb7B); zinc finger CCCH type containing 8 (Zc3h8); zinc finger CCCH type containing 12A (Zc3h12a); zinc finger CCCH type containing 12D (Zc3h12d); zinc finger E-box binding homeobox 1 (Zeb1); zinc finger protein 36, C3H type (Zfp36); zinc finger protein 36, C3H type-like 1 (Zfp36L1); zinc finger protein 36, C3H type-like 2 (Zfp36L2); and zinc finger protein 683 (Zfp683).

In some embodiments, an immune cell comprises a chimeric antigen receptor and one or more edited genes, a regulatory element thereof, or combinations thereof. An edited gene may be an immune response regulation gene, an immunogenic gene, a checkpoint inhibitor gene, a gene involved in immune responses, a cell surface marker, e.g. a T cell surface marker, or any combination thereof. In some embodiments, an immune cell comprises a chimeric antigen receptor and an edited gene that is associated with activated T cell proliferation, for example, Fyn, Itgad, Itga1, Itgam, Itgb2, Satb1, or, Ephb6, a regulatory elements thereof, or combinations thereof. In some embodiments, an immune cell comprises a chimeric antigen receptor and an edited gene that is associated with alpha-beta T cell activation, for example, Dock2, Rorc, Lef1, or TCF7, their regulatory elements thereof, or combinations thereof. In some embodiments, an immune cell comprises a chimeric antigen receptor and an edited gene that is associated with gamma-delta T cell activation, for example, Jag2, Sox13, Mill2, or Jam1, their regulatory elements thereof, or combinations thereof. In some embodiments, an immune cell comprises a chimeric antigen receptor and an edited gene that is associated with positive regulation of T cell proliferation, for example, Cd24a, Cd86, Epo, Fadd, Icos1, Igf1, Igf2, Igfbp2, Tnfsf4, Tnfsf9, Gpam, Il2, Il2ra, Il4, Stat5a, Stat5b, Gli3, Ihh, Itpkb, Nkap, Shh, Ada, Cd24a, Cd28, Ceacam1, Socs1, Cd83, Cd81, Cd74, Bad, Gata3, interleukin 2, interleukin 2 receptor alpha chain, interleukin 4, interleukin 7, interleukin 12a or FoxP3 or their regulatory elements thereof, or combinations thereof. In some embodiments, an immune cell comprises a chimeric antigen receptor and an edited gene that is negative regulation of T-helper cell proliferation or differentiation, for example, Xcl1, Jak3, Rc3h1, Rc3h2, Tbx21, Zbtb7b, Tbx21, Zc3h12a, Smad3, Loxl3, Socs5, Zfp35, or Bcl6 or their regulatory elements thereof, or combinations thereof. In some embodiments, the edited gene may be a checkpoint inhibitor gene, for example, such as a PD1 gene, a PDL1 gene, or a member related to or regulating the pathway of their formation or activation.

In some embodiments, provided herein is an immune cell with an edited TRAC gene (wherein, the TRAC gene may comprise one, two, three, four, five, six, seven eight, nine, ten or more base edits), such that the immune cell does not express an endogenous functional T cell receptor alpha chain. In some embodiments, the immune cell is a T cell expressing a chimeric antigen receptor (a CAR-T cell). In some embodiments, provided herein is a CAR-T cell with base edits in TRAC gene, such that the CAR-T cell have reduced or negligible or no expression of endogenous T cell receptor alpha protein.

In some embodiments, the immune cell comprises an edited TRAC gene, and additionally, at least one edited gene. The at least one edited gene may be selected from the list of genes mentioned in the preceding paragraphs. In one embodiment, the immune cell may comprise an edited TRAC gene, an edited PDCD1 gene, an edited CD52 gene, an edited CD7 gene, an edited B2M gene, an edited CD5 gene, an edited CBLB gene, or any combination thereof. In some embodiments, a single modification event (such as electroporation), may introduce one or more gene edits. In some embodiments at least four, five, six, seven, eight, nine, ten, eleven, twelve, thirteen, fourteen, fifteen, sixteen, seventeen, eighteen, nineteen, twenty or more edits may be introduced in one or more genes simultaneously.

In some embodiments, the immune cell comprises an edited TRAC gene, and an edited PDCD1, CD52, CD7, B2M, CD5, or CBLB gene, or a combination thereof. In some embodiments, the immune cell comprises one or more of edited genes, selected from TRAC, PDCD1, CD52, CD7, B2M, CD5, B2M, CD5, and CBLB gene.

In some embodiments, the immune cell may comprise an edited TRAC gene, an edited CD2 gene, an edited CD3 epsilon gene, an edited CD3 gamma gene, an edited CD3 delta gene, an edited CD5 gene, an edited CD7 gene, an edited CD30 gene, an edited CD33 gene, an edited B2M gene, an edited CD52 gene, an edited CD70 gene, an edited CBLB gene, an edited CIITA gene, or any combination thereof.

In some embodiments, provided herein is an immune cell with an edited TRBC1 or TRBC2 gene, such that the immune cell does not express an endogenous functional T cell receptor beta chain. In some embodiments, provided herein is a CAR-T cell with an edited TRBC1/TRBC2 gene, such that the CAR-T cell exhibits reduced or negligible expression or no expression of endogenous T cell receptor beta chain.

In some embodiments, the immune cell comprises an edited TRBC1/TRBC2 gene, and additionally, at least edited gene. The at least one edited gene may be selected from the list of genes mentioned in the preceding paragraphs. In some embodiments, the immune cell comprises an edited TRBC1/TRBC2 gene, and an edited PDCD1, CD52 or CD7 gene, or a combination thereof. In some embodiments, the CAR-T cell comprises one or more of base edited genes, selected from TRBC1/TRBC2 gene, PDCD1, CD52, and CD7 genes. In some embodiments, each edited gene may comprise a single base edit. In some embodiments, each edited gene may comprise multiple base edits at different regions of the gene.

In some embodiments, the immune cell comprises an edited TRBC1/TRBC2 genes, and an edited PDCD1, CD52, CD7, B2M, CD5, or CBLB gene, or a combination thereof. In some embodiments, the immune cell may be a CAR-T cell. In some embodiments, the CAR-T cell comprises one or more edited gene, selected from TRBC1/TRBC2, PDCD1, CD52, CD7, B2M, CD5, B2M, CD5, and CBLB gene.

In some embodiments, the immune cell may comprise an edited TRBC1/TRBC2 gene, an edited CD2 gene, an edited CD3 epsilon gene, an edited CD3 gamma gene, an edited CD3 delta gene, an edited CD5 gene, an edited CD7 gene, an edited CD30 gene, an edited CD33 gene, an edited B2M gene, an edited CD52 gene, an edited CD70 gene, an edited CBLB gene, an edited CIITA gene, or any combination thereof.

In some embodiments, an immune cell comprises a chimeric antigen receptor and an edited TRAC, B2M, PDCD1, CBLB gene, or a combination thereof, wherein expression of the edited gene is either knocked out or knocked down. In some embodiments, an immune cell comprises a chimeric antigen receptor and an edited TRAC gene, wherein expression of the edited gene is knocked out or knocked down. In some embodiments, an immune cell comprises a chimeric antigen receptor and edited TRAC and B2M genes, wherein expression of the edited genes is either knocked out or knocked down. In some embodiments, an immune cell comprises a chimeric antigen receptor and edited TRAC and PDCD1 genes, wherein expression of the edited genes is either knocked out or knocked down. In some embodiments, an immune cell comprises a chimeric antigen receptor and edited TRAC and CBLB genes, wherein expression of the edited genes is either knocked out or knocked down. In some embodiments, an immune cell comprises a chimeric antigen receptor and edited TRAC, B2M, and PDCD1 genes, wherein expression of the edited genes is either knocked out or knocked down. In some embodiments, an immune cell comprises a chimeric antigen receptor and edited TRAC, B2M, and CBLB genes, wherein expression of the edited genes is either knocked out or knocked down. In some embodiments, an immune cell or immune effector cell comprises a chimeric antigen receptor and edited TRAC, PDCD1, and CBLB genes, wherein expression of the edited genes is either knocked out or knocked down. In some embodiments, an immune cell comprises a chimeric antigen and edited TRAC, B2M, PDCD1, and CBLB genes, wherein expression of the edited genes is either knocked out or knocked down. In some embodiments, an immune cell comprises a chimeric antigen receptor and an edited B2M gene, wherein expression of the edited genes is either knocked out or knocked down. In some embodiments, an immune cell comprises a chimeric antigen receptor and edited B2M and PDCD1 genes, wherein expression of the edited genes is either knocked out or knocked down. In some embodiments, an immune cell comprises a chimeric antigen receptor and edited B2M and CBLB genes, wherein expression of the edited genes is either knocked out or knocked down. In some embodiments, an immune cell comprises a chimeric antigen receptor and edited B2M, PDCD1, and CBLB genes, wherein expression of the edited genes is either knocked out or knocked down. In some embodiments, an immune cell comprises a chimeric antigen receptor and an edited PDCD gene, wherein expression of the edited genes is either knocked out or knocked down. In some embodiments, an immune cell comprises a chimeric antigen receptor and edited PDCD1 and CBLB genes, wherein expression of the edited genes is either knocked out or knocked down. In some embodiments, an immune cell comprises a chimeric antigen receptor and an edited CBLB, expression of the edited gene is either knocked out or knocked down.

In some embodiments, an immune cell comprises a chimeric antigen receptor and an edited TRAC, an edited CD2 gene, an edited CD3 epsilon gene, an edited CD3 gamma gene, an edited CD3 delta gene, an edited CD5 gene, an edited CD7 gene, an edited CD30 gene, an edited CD33 gene, an edited B2M gene, an edited CD52 gene, an edited CD70 gene, an edited CBLB gene, an edited CIITA gene, or any combination thereof, wherein expression of the edited gene is either knocked out or knocked down.

In some embodiments, an immune cell comprises a chimeric antigen receptor and an edited TRBC1 or TRBC2 gene, an edited CD2 gene, an edited CD3 epsilon gene, an edited CD3 gamma gene, an edited CD3 delta gene, an edited CD5 gene, an edited CD7 gene, an edited CD30 gene, an edited CD33 gene, an edited B2M gene, an edited CD52 gene, an edited CD70 gene, an edited CBLB gene, an edited CIITA gene, or any combination thereof, wherein expression of the edited gene is either knocked out or knocked down.

In some embodiments, an immune cell, including but not limited to any immune cell comprising an edited gene selected from any of the aforementioned gene edits, can be edited to generate mutations in other genes that enhance the CAR-T's function or reduce immunosuppression or inhibition of the cell. For example, in some embodiments, an immune cell comprises a chimeric antigen receptor and an edited TGFBR2, ZAP70, NFATc1, TET2 gene, or a combination thereof, wherein expression of the edited gene is either knocked out or knocked down. In some embodiments, an immune cell comprises a chimeric antigen receptor and an edited TGFBR2 gene, wherein expression of the edited gene is knocked out or knocked down. In some embodiments, an immune cell comprises a chimeric antigen receptor and edited TGFBR2 and ZAP70 genes, wherein expression of the edited genes is either knocked out or knocked down. In some embodiments, an immune cell comprises a chimeric antigen receptor and edited TGFBR2 and ZAP70 genes, wherein expression of the edited genes is either knocked out or knocked down. In some embodiments, an immune cell comprises a chimeric antigen receptor and edited TGFBR2 and NFATC1 genes, wherein expression of the edited genes is either knocked out or knocked down. In some embodiments, an immune cell comprises a chimeric antigen receptor and edited TGFBR2 and TET2 genes, wherein expression of the edited genes is either knocked out or knocked down. In some embodiments, an immune cell comprises a chimeric antigen receptor and edited TGFBR2, ZAP70, and NFATC1 genes, wherein expression of the edited genes is either knocked out or knocked down. In some embodiments, an immune cell comprises a chimeric antigen receptor and edited TGFBR2, ZAP70, and TET2 genes, wherein expression of the edited genes is either knocked out or knocked down. In some embodiments, an immune cell comprises a chimeric antigen receptor and edited TGFBR2, NFATC1, and TET2 genes, wherein expression of the edited genes is either knocked out or knocked down. In some embodiments, an immune cell comprises a chimeric antigen and edited TGFBR2, ZAP70, NFATC1, and TET2 genes, wherein expression of the edited genes is either knocked out or knocked down. In some embodiments, an immune cell comprises a chimeric antigen receptor and an edited ZAP70 gene, wherein expression of the edited genes is either knocked out or knocked down. In some embodiments, an immune cell comprises a chimeric antigen receptor and edited ZAP70 and NFATC1 genes, wherein expression of the edited genes is either knocked out or knocked down. In some embodiments, an immune cell comprises a chimeric antigen receptor and edited ZAP70 and TET2 genes, wherein expression of the edited genes is either knocked out or knocked down. In some embodiments, an immune cell comprises a chimeric antigen receptor and edited ZAP70, PDCD1, and TET2 genes, wherein expression of the edited genes is either knocked out or knocked down. In some embodiments, an immune cell comprises a chimeric antigen receptor and an edited PCDC1 gene, wherein expression of the edited genes is either knocked out or knocked down. In some embodiments, an immune cell comprises a chimeric antigen receptor and edited PCDC1 and TET2 genes, wherein expression of the edited genes is either knocked out or knocked down. And in some embodiments, an immune cell comprises a chimeric antigen receptor and an edited TET2, expression of the edited gene is either knocked out or knocked down.

Editing of Target Genes in Immune Cells

In some embodiments, provided herein is an immune cell with at least one modification in an endogenous gene or regulatory elements thereof. In some embodiments, the immune cell may comprise at least one modification in each of at least two, at least three, four, five, six, seven, eight, nine, ten, eleven, twelve, thirteen, fourteen, fifteen, sixteen, seventeen, eighteen, nineteen, twenty or more endogenous genes or regulatory elements thereof. In some embodiments, the at least one modification is a single nucleobase modification. In some embodiments, the at least one modification is by base editing. The base editing may be positioned at any suitable position of the gene, or in a regulatory element of the gene. Thus, it may be appreciated that a single base editing at a start codon, for example, can completely abolish the expression of the gene. In some embodiments, the base editing may be performed at a site within an exon. In some embodiments, the base editing may be performed at a site on more than one exons. In some embodiments, the base editing may be performed at any exon of the multiple exons in a gene. In some embodiments, base editing may introduce a premature STOP codon into an exon, resulting in either lack of a translated product or in a truncated that may be misfolded and thereby eliminated by degradation, or may produce an unstable mRNA that is readily degraded. In some embodiments, the immune cell is a T cell. In some embodiments, the immune cell is a CAR-T cell.

In some embodiments, base editing may be performed, for example on exon 1, or exon 2, or exon 3 or exon 4 of human TRAC gene (UCSC genomic database ENSG00000277734.8). In some embodiments, base editing in human TRAC gene is performed at a site within exon 1. In some embodiments, base editing in human TRAC gene is performed at a site within exon 2. In some embodiments, base editing in human TRAC gene is performed at a site within exon 3. In some embodiments, base editing in human TRAC gene is performed at a site within exon 4. In some embodiments one or more base editing actions can be performed on human TRAC gene, at exon 1, exon 2, exon 3, exon 4 or any combination thereof.

For example, base editing may be performed on exon 1, or exon 2, or exon 3 or exon 4, of human B2M gene (Chromosome 15, NC_000015.10, 44711492-44718877; exemplary mRNA sequence NM_004048). In some embodiments, base editing in human B2M gene is performed at a site within exon 1. In some embodiments, base editing in human B2M gene is performed at a site within exon 2. In some embodiments, base editing in human B2M gene is performed at a site within exon 3. In some embodiments, base editing in human B2M gene is performed at a site within exon 4. In some embodiments one or more base editing actions can be performed on human B2M gene, at exon 1, exon 2, exon 3, exon 4 or any combination thereof.

In some embodiments, base editing may be performed on an intron. For example, base editing may be performed on an intron. In some embodiments, the base editing may be performed at a site within an intron. In some embodiments, the base editing may be performed at a site on more than one introns. In some embodiments, the base editing may be performed at any exon of the multiple introns in a gene. In some embodiments, one or more base editing may be performed on an exon, an intron or any combination of exons and introns.

For example, base editing may be performed, for example on any one or more of the introns in human TRAC gene. In some embodiments, base editing in human TRAC gene is performed at a site within intron 1. In some embodiments, base editing in human TRAC gene is performed at a site within intron 2. In some embodiments, base editing in human TRAC gene is performed at a site within intron 3. In some embodiments one or more base editing actions can be performed on human TRAC gene, at exon 1, exon 2, exon 3, exon 4, intron 1, intron 2, intron 3, or any combination thereof. In some embodiments one or more base edits can be performed on the last noncoding exon of human TRAC gene.

In some embodiments, the modification or base edit may be within a promoter site. In some embodiments, the base edit may be introduced within an alternative promoter site. In some embodiments, the base edit may be in a 5′ regulatory element, such as an enhancer. In some embodiment, base editing may be introduced to disrupt the binding site of a nucleic acid binding protein. Exemplary nucleic acid binding proteins may be a polymerase, nuclease, gyrase, topoisomerase, methylase or methyl transferase, transcription factors, enhancer, PABP, zinc finger proteins, among many others.

In some embodiments, base editing may generate a splice acceptor-splice donor (SA-SD) site. For example, targeted base editing generating a SA-SD, or at a SA-SD site can result in reduced expression of a gene. For example, exon 1 SD site of TRAC at C5 may be targeted for base editing (GT-AT); TRAC exon 3 SA disruption may be targeted (AG-AA); B2M exon 1 SD at C6 position may be disrupted by base editing (GT-AT); B2M exon 3 SA at C6 can be targeted (AG-AA).

In some embodiments, provided herein is an immune cell with at least one modification in one or more endogenous genes. In some embodiments, the immune cell may have at least one modification in one, two, three, four, five, six, seven, eight, nine, ten, eleven, twelve, thirteen, fourteen, fifteen, sixteen, seventeen, eighteen, nineteen, twenty or more endogenous genes. In some embodiments, the modification generates a premature stop codon in the endogenous genes. In some embodiments, the modification is a single base modification. In some embodiments, the modification is generated by base editing. The premature stop codon may be generated in an exon, an intron, or an untranslated region. In some embodiments, base editing may be used to introduce more than one STOP codon, in one or more alternative reading frames. For example, a premature STOP codon can be introduced at exon 3 C4 position of TRAC (CAA-TAA) by base editing.

In some embodiments, modification/base edits may be introduced at a 3′-UTR, for example, in a poly adenylation (poly-A) site. In some embodiments, base editing may be performed on a 5′-UTR region.

Chimeric Antigen Receptor Insertion into Immune Cell Genes

In some embodiments, a chimeric antigen receptor is inserted into the TRAC gene. This has advantages. First, because TRAC is highly expressed in immune cell, the chimeric antigen receptor will be similarly expressed when its construct is designed to insert the chimeric antigen receptor into the TRAC gene such that expression of the receptor is driven by the TRAC promoter. Second, inserting the chimeric antigen receptor into the TRAC gene will knockout TRAC expression. In some embodiments, the gene editing system described herein can be used to insert the chimeric antigen receptor into the TRAC locus. gRNAs specific for the TRAC locus can guide the gene editing system to the locus and initiate double-stranded DNA cleavage. In particular embodiments, the gRNA is used in conjunction with Cas12b. In various embodiments, the gene editing system is used in conjunction with a nucleic acid having a sequence encoding a CAR receptor. Exemplary guide RNAs are provided in the following Table 1A.

TABLE 1A

| gRNA sequence | PAM | napDNAbp | Gene Exon |
|---|---|---|---|
| GUUCUGUCUUUUGGUCAGGACAACCGUC | ATTN | BhCas 12b | TRAC KO |
| UAGCUAUAAGUGCUGCAGGGUGUGAGAA | | nuclease | gRNA 1 |
| ACUCCUAUUGCUGGACGAUGUCUCUUAC | | | (Exon 1) |
| GAGGCAUUAGCACAGAGUCUCUCAGCUG | | | |
| GUACAC | | | |
| GUUCUGUCUUUUGGUCAGGACAACCGUC | ATTN | BhCas 12b | TRAC KO |
| UAGCUAUAAGUGCUGCAGGGUGUGAGGA | | nuclease | gRNA 2 |
| AACUCCUAUUGCUGGACGAUGUCUCUUA | | | (Exon 1) |
| CGAGGCAUUAGCACACCGAUUUUGAUUC | | | |
| UCAAACA | | | |
| GUUCUGUCUUUUGGUCAGGACAACCGUC | ATTN | BhCas 12b | TRAC KO |
| UAGCUAUAAGUGCUGCAGGGUGUGAGAA | | nuclease | gRNA 3 |
| ACUCCUAUUGCUGGACGAUGUCUCUUAC | | | (Exon 1) |
| GAGGCAUUAGCACUCAAACAAAUGUGCA | | | |
| CAAAG | | | |
| GUUCUGUCUUUUGGUCAGGACAACCGUC | ATTN | BhCas 12b | TRAC KO |
| UAGCUAUAAGUGCUGCAGGGUGUGAGAA | | nuclease | gRNA 4 |
| ACUCCUAUUGCUGGACGAUGUCUCUUAC | | | (Exon 1) |
| GAGGCAUUAGCACUCAAACAAAUGUGUC | | | |
| ACAAAG | | | |
| GUUCUGUCUUUUGGUCAGGACAACCGUC | ATTN | BhCas 12b | TRAC KO |
| UAGCUAUAAGUGCUGCAGGGUGUGAGAA | | nuclease | gRNA 5 |
| ACUCCUAUUGCUGGACGAUGUCUCUUAC | | | (Exon 1) |
| GAGGCAUUAGCACUUUGAGAAUCAAAAU | | | |
| CGGUA | | | |
| GUUCUGUCUUUUGGUCAGGACAACCGUC | ATTN | BhCas 12b | TRAC KO |
| UAGCUAUAAGUGCUGCAGGGUGUGAGAA | | nuclease | gRNA 6 |
| ACUCCUAUUGCUGGACGAUGUCUCUUAC | | | (Exon 1) |
| GAGGCAUUAGCACUGAUGUGUAUAUCAC | | | |
| AGACAA | | | |
| GUUCUGUCUUUUGGUCAGGACAACCGUC | ATTN | BhCas 12b | TRAC KO |
| UAGCUAUAAGUGCUGCAGGGUGUGAGAA | | nuclease | gRNA 7 |
| ACUCCUAUUGCUGGACGAUGUCUCUUAC | | | (Exon 1) |
| GAGGCAUUAGCAGUUGCUCCAGGCCACA | | | |
| GCAU | | | |
| GUUCUGUCUUUUGGUCAGGACAACCGUC | ATTN | BhCas 12b | TRAC KO |
| UAGCUAUAAGUGCUGCAGGGUGUGAGAA | | nuclease | gRNA 8 |
| ACUCCUAUUGCUGGACGAUGUCUCUUAC | | | (Exon 1) |
| GAGGCAUUAGCACUUCCAGAAGACACCU | | | |
| UCUUCC | | | |
| GUUCUGUCUUUUGGUCAGGACAACCGUC | ATTN | BhCas 12b | TRAC KO |
| UAGCUAUAAGUGCUGCAGGGUGUGAGAA | | nuclease | gRNA 9 |
| ACUCCUAUUGCUGGACGAUGUCUCUUAC | | | (Exon 1) |
| GAGGCAUUAGCACCAGAAGACACCUUCU | | | |
| UCCCCA | | | |
| GUUCUGUCUUUUGGUCAGGACAACCGUC | ATTN | BhCas 12b | TRAC KO |
| UAGCUAUAAGUGCUGCAGGGUGUGAGAG | | nuclease | gRNA 10 |
| AAACUCCUAUUGCUGGACGAUGUCUCUU | | | (Exon 3) |
| ACGAGGCAUUAGCACGGUUCCGAAUCCU | | | |
| CCUGA | | | |
| GUUCUGUCUUUUGGUCAGGACAACCGUC | ATTN | BhCas 12b | TRAC KO |
| UAGCUAUAAGUGCUGCAGGGUGUGAGAA | | nuclease | gRNA 11 |
| ACUCCUAUUGCUGGACGAUGUCUCUUAC | | | (Exon 3) |
| GAGGCAUUAGCACGGAACCCAAUCACUG | | | |
| ACAGGU | | | |

A DNA construct encoding the chimeric antigen receptor and nucleic acid containing extended stretches of TRAC DNA that flank the gRNA targeting sequences. Without being bound by theory, the construct binds to the complementary TRAC sequences, and the chimeric antigen receptor DNA, residing in proximity to the TRAC sequences on the construct is then inserted at the site of the lesion, effectively knocking out the TRAC gene and knocking in the chimeric antigen receptor nucleic acid. Table 1 provides guide RNAs for the TRAC gene that can guide the base editing machinery to the TRAC locus, which enables insertion of the chimeric antigen receptor nucleic acid. The first 11 gRNAS are for BhCas12b nuclease. The second set of 11 are for the BvCas12b nuclease. These are all for inserting the CAR at TRAC by creating a double stranded break, and not for base editing.

TABLE 1B
TRAC guide RNAs

| Guide RNA Target | Guide RNA Spacer | Gene Exon |
|---|---|---|
| GTTCTGTCTTTTGGTCAGG | GUUCUGUCUUUUGGUCAG | TRAC KO |
| ACAACCGTCTAGCTATAAG | GACAACCGUCUAGCUAUA | gRNA 1 |
| TGCTGCAGGGTGTGAGAAA | AGUGCUGCAGGGUGUGAG | |
| CTCCTATTGCTGGACGATG | AAACUCCUAUUGCUGGAC | |
| TCTCTTACGAGGCATTAGC | GAUGUCUCUUACGAGGCA | |
| ACAGAGTCTCTCAGCTGGT | UUAGCACAGAGUCUCUCA | |
| ACA | GCUGGUACA | |
| GTTCTGTCTTTTGGTCAGG | GUUCUGUCUUUUGGUCAG | TRAC KO |
| ACAACCGTCTAGCTATAAG | GACAACCGUCUAGCUAUA | gRNA 2 |
| TGCTGCAGGGTGTGAGAAA | AGUGCUGCAGGGUGUGAG | |
| CTCCTATTGCTGGACGATG | AAACUCCUAUUGCUGGAC | |
| TCTCTTACGAGGCATTAGC | GAUGUCUCUUACGAGGCA | |
| ACACCGATTTTGATTCTCA | UUAGCACACCGAUUUUGA | |
| AAC | UUCUCAAAC | |
| GTTCTGTCTTTTGGTCAGG | GUUCUGUCUUUUGGUCAG | TRAC KO |
| ACAACCGTCTAGCTATAAG | GACAACCGUCUAGCUAUA | gRNA 3 |
| TGCTGCAGGGTGTGAGAAA | AGUGCUGCAGGGUGUGAG | |
| CTCCTATTGCTGGACGATG | AAACUCCUAUUGCUGGAC | |
| TCTCTTACGAGGCATTAGC | GAUGUCUCUUACGAGGCA | |
| ACTGATTCTCAAACAAATG | UUAGCACUGAUUCUCAAA | |
| TGT | CAAAUGUGU | |
| GTTCTGTCTTTTGGTCAGG | GUUCUGUCUUUUGGUCAG | TRAC KO |
| ACAACCGTCTAGCTATAAG | GACAACCGUCUAGCUAUA | gRNA 4 |
| TGCTGCAGGGTGTGAGAAA | AGUGCUGCAGGGUGUGAG | |
| CTCCTATTGCTGGACGATG | AAACUCCUAUUGCUGGAC | |
| TCTCTTACGAGGCATTAGC | GAUGUCUCUUACGAGGCA | |
| ACTCAAACAAATGTGTCAC | UUAGCACUCAAACAAAUG | |
| AAA | UGUCACAAA | |
| GTTCTGTCTTTTGGTCAGG | GUUCUGUCUUUUGGUCAG | TRAC KO |
| ACAACCGTCTAGCTATAAG | GACAACCGUCUAGCUAUA | gRNA 5 |
| TGCTGCAGGGTGTGAGAAA | AGUGCUGCAGGGUGUGAG | |
| CTCCTATTGCTGGACGATG | AAACUCCUAUUGCUGGAC | |
| TCTCTTACGAGGCATTAGC | GAUGUCUCUUACGAGGCA | |
| ACGTTTGAGAATCAAAATC | UUAGCACGUUUGAGAAUC | |
| GGT | AAAAUCGGU | |
| GTTCTGTCTTTTGGTCAGG | GUUCUGUCUUUUGGUCAG | TRAC KO |
| ACAACCGTCTAGCTATAAG | GACAACCGUCUAGCUAUA | gRNA 6 |
| TGCTGCAGGGTGTGAGAAA | AGUGCUGCAGGGUGUGAG | |
| CTCCTATTGCTGGACGATG | AAACUCCUAUUGCUGGAC | |
| TCTCTTACGAGGCATTAGC | GAUGUCUCUUACGAGGCA | |
| ACTGATGTGTATATCACAG | UUAGCACUGAUGUGUAUA | |
| ACA | UCACAGACA | |
| GTTCTGTCTTTTGGTCAGG | GUUCUGUCUUUUGGUCAG | TRAC KO |
| ACAACCGTCTAGCTATAAG | GACAACCGUCUAGCUAUA | gRNA 7 |
| TGCTGCAGGGTGTGAGAAA | AGUGCUGCAGGGUGUGAG | |
| CTCCTATTGCTGGACGATG | AAACUCCUAUUGCUGGAC | |
| TCTCTTACGAGGCATTAGC | GAUGUCUCUUACGAGGCA | |
| ACGTTGCTCCAGGCCACAG | UUAGCACGUUGCUCCAGG | |
| CAC | CCACAGCAC | |
| GTTCTGTCTTTTGGTCAGG | GUUCUGUCUUUUGGUCAG | TRAC KO |
| ACAACCGTCTAGCTATAAG | GACAACCGUCUAGCUAUA | gRNA 8 |
| TGCTGCAGGGTGTGAGAAA | AGUGCUGCAGGGUGUGAG | |
| CTCCTATTGCTGGACGATG | AAACUCCUAUUGCUGGAC | |
| TCTCTTACGAGGCATTAGC | GAUGUCUCUUACGAGGCA | |
| ACTTCCAGAAGACACCTTC | UUAGCACUUCCAGAAGAC | |
| TTC | ACCUUCUUC | |
| GTTCTGTCTTTTGGTCAGG | GUUCUGUCUUUUGGUCAG | TRAC KO |
| ACAACCGTCTAGCTATAAG | GACAACCGUCUAGCUAUA | gRNA 9 |
| TGCTGCAGGGTGTGAGAAA | AGUGCUGCAGGGUGUGAG | |
| CTCCTATTGCTGGACGATG | AAACUCCUAUUGCUGGAC | |
| TCTCTTACGAGGCATTAGC | GAUGUCUCUUACGAGGCA | |
| ACCAGAAGACACCTTCTTC | UUAGCACCAGAAGACACC | |
| CCC | UUCUUCCCC | |
| GTTCTGTCTTTTGGTCAGG | GUUCUGUCUUUUGGUCAG | TRAC KO |
| ACAACCGTCTAGCTATAAG | GACAACCGUCUAGCUAUA | gRNA 10 |
| TGCTGCAGGGTGTGAGAAA | AGUGCUGCAGGGUGUGAG | |
| CTCCTATTGCTGGACGATG | AAACUCCUAUUGCUGGAC | |
| TCTCTTACGAGGCATTAGC | GAUGUCUCUUACGAGGCA | |
| ACGGTTCCGAATCCTCCTC | UUAGCACGGUUCCGAAUC | |
| CTG | CUCCUCCUG | |
| GTTCTGTCTTTTGGTCAGG | GUUCUGUCUUUUGGUCAG | TRAC KO |
| ACAACCGTCTAGCTATAAG | GACAACCGUCUAGCUAUA | gRNA 11 |
| TGCTGCAGGGTGTGAGAAA | AGUGCUGCAGGGUGUGAG | |
| CTCCTATTGCTGGACGATG | AAACUCCUAUUGCUGGAC | |
| TCTCTTACGAGGCATTAGC | GAUGUCUCUUACGAGGCA | |
| ACGGAACCCAATCACTGAC | UUAGCACGGAACCCAAUC | |
| AGG | ACUGACAGG | |
| GACCTATAGGGTCAATGAA | GACCUAUAGGGUCAAUGA | TRAC KO |
| TCTGTGCGTGTGCCATAAG | AUCUGUGCGUGUGCCAUA | gRNA 1 |
| TAATTAAAAATTACCCACC | AGUAAUUAAAAAUUACCC | |
| ACAGGAGCACCTGAAAACA | ACCACAGGAGCACCUGAA | |
| GGTGCTTGGCACAGAGTCT | AACAGGUGCUUGGCACAG | |
| CTCAGCTGGTACA | AGUCUCUCAGCUGGUACA | |
| GACCTATAGGGTCAATGAA | GACCUAUAGGGUCAAUGA | TRAC KO |
| TCTGTGCGTGTGCCATAAG | AUCUGUGCGUGUGCCAUA | gRNA 2 |
| TAATTAAAAATTACCCACC | AGUAAUUAAAAAUUACCC | |
| ACAGGAGCACCTGAAAACA | ACCACAGGAGCACCUGAA | |
| GGTGCTTGGCACACCGATT | AACAGGUGCUUGGCACAC | |
| TTGATTCTCAAAC | CGAUUUUGAUUCUCAAAC | |
| GACCTATAGGGTCAATGAA | GACCUAUAGGGUCAAUGA | TRAC KO |
| TCTGTGCGTGTGCCATAAG | AUCUGUGCGUGUGCCAUA | gRNA 3 |
| TAATTAAAAATTACCCACC | AGUAAUUAAAAAUUACCC | |
| ACAGGAGCACCTGAAAACA | ACCACAGGAGCACCUGAA | |
| GGTGCTTGGCACTGATTCT | AACAGGUGCUUGGCACUG | |
| CAAACAAATGTGT | AUUCUCAAACAAAUGUGU | |
| GACCTATAGGGTCAATGAA | GACCUAUAGGGUCAAUGA | TRAC KO |
| TCTGTGCGTGTGCCATAAG | AUCUGUGCGUGUGCCAUA | gRNA 4 |
| TAATTAAAAATTACCCACC | AGUAAUUAAAAAUUACCC | |
| ACAGGAGCACCTGAAAACA | ACCACAGGAGCACCUGAA | |
| GGTGCTTGGCACTCAAACA | AACAGGUGCUUGGCACUC | |
| AATGTGTCACAAA | AAACAAAUGUGUCACAAA | |
| GACCTATAGGGTCAATGAA | GACCUAUAGGGUCAAUGA | TRAC KO |
| TCTGTGCGTGTGCCATAAG | AUCUGUGCGUGUGCCAUA | gRNA 5 |
| TAATTAAAAATTACCCACC | AGUAAUUAAAAAUUACCC | |
| ACAGGAGCACCTGAAAACA | ACCACAGGAGCACCUGAA | |
| GGTGCTTGGCACGTTTGAG | AACAGGUGCUUGGCACGU | |
| AATCAAAATCGGT | UUGAGAAUCAAAAUCGGU | |
| GACCTATAGGGTCAATGAA | GACCUAUAGGGUCAAUGA | TRAC KO |
| TCTGTGCGTGTGCCATAAG | AUCUGUGCGUGUGCCAUA | gRNA 6 |
| TAATTAAAAATTACCCACC | AGUAAUUAAAAAUUACCC | |
| ACAGGAGCACCTGAAAACA | ACCACAGGAGCACCUGAA | |
| GGTGCTTGGCACTGATGTG | AACAGGUGCUUGGCACUG | |
| TATATCACAGACA | AUGUGUAUAUCACAGACA | |
| GACCTATAGGGTCAATGAA | GACCUAUAGGGUCAAUGA | TRAC KO |
| TCTGTGCGTGTGCCATAAG | AUCUGUGCUGUGUGCCAU | gRNA 7 |
| TAATTAAAAATTACCCACC | AAGUAAUUAAAAAUUACC | |
| ACAGGAGCACCTGAAAACA | CACCACAGGAGCACCUGA | |
| GGTGCTTGGCACGTTGCTC | AAACAGGUGCUUGGCACG | |
| CAGGCCACAGCAC | UUGCUCCAGGCCACAGCA | |
| | C | |
| GACCTATAGGGTCAATGAA | GACCUAUAGGGUCAAUGA | TRAC KO |
| TCTGTGCGTGTGCCATAAG | AUCUGUGCGUGUGCCAUA | gRNA 8 |
| TAATTAAAAATTACCCACC | AGUAAUUAAAAAUUACCC | |
| ACAGGAGCACCTGAAAACA | ACCACAGGAGCACCUGAA | |
| GGTGCTTGGCACTTCCAGA | AACAGGUGCUUGGCACUU | |
| AGACACCTTCTTC | CCAGAAGACACCUUCUUC | |
| GACCTATAGGGTCAATGAA | GACCUAUAGGGUCAAUGA | TRAC KO |
| TCTGTGCGTGTGCCATAAG | AUCUGUGCGUGUGCCAUA | gRNA 9 |
| TAATTAAAAATTACCCACC | AGUAAUUAAAAAUUACCC | |
| ACAGGAGCACCTGAAAACA | ACCACAGGAGCACCUGAA | |
| GGTGCTTGGCACCAGAAGA | AACAGGUGCUUGGCACCA | |
| CACCTTCTTCCCC | GAAGACACCUUCUUCCCC | |
| GACCTATAGGGTCAATGAA | GACCUAUAGGGUCAAUGA | TRAC KO |
| TCTGTGCGTGTGCCATAAG | AUCUGUGCGUGUGCCAUA | gRNA 10 |
| TAATTAAAAATTACCCACC | AGUAAUUAAAAAUUACCC | |
| ACAGGAGCACCTGAAAACA | ACCACAGGAGCACCUGAA | |
| GGTGCTTGGCACGGTTCCG | AACAGGUGCUUGGCACGG | |
| AATCCTCCTCCTG | UUCCGAAUCCUCCUCCUG | |
| GACCTATAGGGTCAATGAA | GACCUAUAGGGUCAAUGA | TRAC KO |
| TCTGTGCGTGTGCCATAAG | AUCUGUGCGUGUGCCAUA | gRNA 11 |
| TAATTAAAAATTACCCACC | AGUAAUUAAAAAUUACCC | |
| ACAGGAGCACCTGAAAACA | ACCACAGGAGCACCUGAA | |
| GGTGCTTGGCACGGAACCC | AACAGGUGCUUGGCACGG | |
| AATCACTGACAGG | AACCCAAUCACUGACAGG | |

First 11 gRNAs are for BhCas12b nuclease. Second set of 11 gRNAs are for the BvCas12b nuclease. Scaffold sequence in bold, in first instance.

In some embodiments, a nucleic acid encoding a chimeric antigen receptor of the present invention can be targeted to the TRAC locus using the BE4 base editor. In some embodiments, the chimeric antigen receptor is targeted to the TRAC locus using a CRISPR/Cas9 base editing system.

To produce the gene edits described above, immune cells are collected from a subject and contacted with two or more guide RNAs and a nucleobase editor polypeptide comprising a nucleic acid programmable DNA binding protein (napDNAbp) and a cytidine deaminase or adenosine deaminase. In some embodiments, the collected immune cells are contacted with at least one nucleic acid, wherein the at least one nucleic acid encodes two or more guide RNAs and a nucleobase editor polypeptide comprising a nucleic acid programmable DNA binding protein (napDNAbp) and a cytidine deaminase. In some embodiments, the gRNA comprises nucleotide analogs. These nucleotide analogs can inhibit degradation of the gRNA from cellular processes. Table 2 provides target sequences to be used for gRNAs.

TABLE 2
Exemplary Target Sequences

| Target | Target | | | | | |
|---|---|---|---|---|---|---|
| protein | residue | gRNA target | gRNA spacer | BE | Codon change | Residue function |
| NFATC1 | R118 | CTCGATGCGAGGACTCTCCA | CUCGAUGCGAGGACUCUCCA | BE | CGC > CAC | Calcineurin |
| | | | | | | binding |
| | I119 | TCTCGATGCGAGGACTCTCC | UCUCGAUGCGAGGACUCUCC | ABE | ATC > ACC | Calcineurin |
| | | | | | | binding |
| | E120 | CATCGAGATAACCTCGTGCT | CAUCGAGAUAACCUCGUGCU | ABE | GAG > GGG | Calcineurin |
| | | | | | | binding |
| | S172 | TGGCCGGGCTCAGGCACGAG | UGGCCGGGCUCAGGCACGAG | BE | AGC > AAC | PHOSPHORYL ATION |
| | W396 | GCCCACTGGTAGGGGTGCTG | GCCCACUGGUAGGGGUGCUG | ABE | TGG > CGG | Calcineurin |
| | | | | | | binding |
| | R439 | TGGGCTCGGTGGTGGGACTT | UGGGCUCGGUGGUGGGACUU | BE | CGA > CAA | DNA BINDING |
| | H441 | CGAGCCCACTACGAGACGGA | CGAGCCCACUACGAGACGGA | ABE | CAC > CGC | DNA BINDING |
| | Y442 | CTCGTAGTGGGCTCGGTGGT | CUCGUAGUGGGCUCGGUGGU | ABE | TAC > CAC | DNA BINDING |
| | K452 | GCCGTGAAGGCGTCGGCCGG | GCCGUGAAGGCGUCGGCCGG | ABE | AAG > GGG | DNA BINDING |
| | R540 | GTTTCTGAGTTTCAGGATTC | GUUUCUGAGUUUCAGGAUUC | BE | AGA > AAA | DNA BINDING |
| | R555 | CATCGGGAGGAAGAACACAC | CAUCGGGAGGAAGAACACAC | ABE | AGG > GGG | DNA BINDING |
| | K556 | GGAGGAAGAACACACGGGTA | GGAGGAAGAACACACGGGUA | ABE | AAG > GGG | DNA BINDING |
| | Q589 | GAGCGCTGGGCTGCATCAGA | GAGCGCUGGGCUGCAUCAGA | BE | CAG > CAT | DNA BINDING |
| NFATC2 | E114 | TGATCTCGATCCGAGGGCTC | UGAUCUCGAUCCGAGGGCUC | BE | GAG > AAA | Calcineurin |
| | | | | | | binding |
| | I115 | ACGGAGTGATCTCGATCCGA | ACGGAGUGAUCUCGAUCCGA | ABE | ATC > ACC | Calcineurin |
| | | | | | | binding |
| | R253 | GCGGAGGCATTCGTGCGCCG | GCGGAGGCAUUCGUGCGCCG | ABE | AGG > GGG | NLS |
| | S99 | GCCGCGCTCAGAAACTTCTG | GCCGCGCUCAGAAACUUCUG | BE | AGC > AAC | PHOSPHORYL ATION |
| | S107 | GGGCCTCGGGCCTGAGCCCG | GGGCCUCGGGCCUGAGCCCU | BE | TCG > TTG | PHOSPHORYL ATION |
| | S148 | CCTCGGGCTGGCGGCCACCC | CCUCGGGCUGGCGGCCACCC | BE | AGC > AAC | PHOSPHORYL ATION |
| | S236 | CCACTCGCCCGTGCCCCGTC | CCACUCGCCCGUGCCCCGUC | BE | TCG > TTG | PHOSPHORYL ATION |
| | S255 | GCATTCGTGCGCCGAGGCCT | GCAUUCGUGCGCCGAGGCCU | BE | TCG > TTG | PHOSPHORYL ATION |
| | S268 | GAGCCTCACCCCAGCGCTCC | GAGCCUCACCCCAGCGCUCC | BE | TCA > TTA | PHOSPHORYL ATION |
| | S274 | GAGGGGCTCCGGGAGCGCTG | GAGGGGCUCCGGGAGCGCUG | BE | AGC > AAC | PHOSPHORYL ATION |
| | S326 | AGGGCTGGTCTTCCACATCT | AGGGCUGGUCUUCCACAUCU | BE | AGC > AAC | PHOSPHORYL ATION |
| NFATC4 | S213 | GCGGGGAGCCCAGGCCAAAG | GCGGGGAGCCCAGGCCAAAG | ABE | TCC > CCC | PHOSPHORYL ATION |
| AKT1 | T305 | GCCACCATGAAGACCTTTTG | GCCACCAUGAAGACCUUUUG | BE | ACC > ATT | PHOSPHORYL ATION |
| | T312 | TTGCGGCACACCTGAGTACC | UUGCGGCACACCUGAGUACC | BE | ACA > ATA | PHOSPHORYL ATION |
| | S473 | GTAGGAGAACTGGGGGAAGT | GUAGGAGAACUGGGGGAAGU | ABE | TCC > CCC | PHOSPHORYL ATION |
| | Y474 | CTCCTACTCGGCCAGCGGCA | CUCCUACUCGGCCAGCGGCA | ABE | TAC > TGC | PHOSPHORYL ATION |
| AKT2 | T309 | GAAAACCTTCTGTGGGACCC | GAAAACCUUCUGUGGGACCC | BE | ACC > ATT | PHOSPHORYL ATION |
| | S474 | AGTAGGAGAACTGGGGGAAG | AGUAGGAGAACUGGGGGAAG | ABE | TCC > CCC | PHOSPHORYL ATION |
| BLIMP1 | C608 | GTTGCAAGTCTGACATTTGA | GUUGCAAGUCUGACAUUUGA | ABE | TGC > CGC | DNA BINDING |
| | (ZF2) | | | | | |
| | C608 | GTTGCAAGTCTGACATTTGA | GUUGCAAGUCUGACAUUUGA | BE | TGC > TAC | DNA BINDING |
| | (ZF2) | | | | | |
| | H621 | GAAACACTACCTGGTACACA | GAACACUACCUGGUACACA | BE | CAC > TAT | DNA BINDING |
| | (ZF2) | | | | | |
| | C636 | TGTGGCAGACCTACAGTGTA | UGUGGCAGACCUACAGUGUA | BE | TGC > TAC | DNA BINDING |
| | (ZF3) | | | | | |
| | C664 | GGGCACACCTTGCATTGGTA | GGGCACACCUUGCAUUGGUA | ABE | TGC > CGC | DNA BINDING |
| | (ZF4) | | | | | |
| | Splice | CTGCGCACCTGGCATTCATG | CUGCGCACCUGGCAUUCAUG | BE | | |
| | site 1 | | | | | |
| GCN2 | Exon | CCTACCGGTCCGCAAGCGTC | CCUACCGGUCCGCAAGUGUC | BE | | KNOCKOUT |
| kinase | 1 SD | | | | | |
| (IDO | Exon | ACTCACACATCTGGATAGGT | ACUCACACAUCUGGAUAGGU | BE | | KNOCKOUT |
| pathway) | 2 SD | | | | | |
| | Exon | GACTTACCTAGACCTTCCTG | GACUUACCUAGACCUUCCUG | BE | | KNOCKOUT |
| | 5 SD | | | | | |
| CBL-B | C373 | AATCTTACAGAGCTGAAAAG | AAUCUUACAGAGCUGAAAAG | BE | TGT > TAT | E3 UBIQUITIN |
| | | | | | | LIGASE |
| | Y665.1 | CATCATATTCTTCACTTCCA | CAUCAUAUUCUUCACUUCCA | ABE | TAT > TAC | |
| | Y665.2 | AAGAATATGATGTTCCTCCC | AAGAAUAUGAUGUUCCUCCC | ABE | TAT > TGT | |
| | K907 | CCCCTAAACCACGACCGCGC | CCCCUAAACCACGACCGCGC | ABE | AAA > GGG | |
| | R911 | TCCTGCGCGGTCGTGGTTTA | UCCUGCGCGGUCGUGGUUUA | BE | CGC > CAC | |
| SHP1 | Y377 | CCCTACTCTGTGACCAACTG | CCCUACUCUGUGACCAACUG | ABE | TAC > TGC | |
| IRF4 | R96 | CGCAGGCGCGTCTTCCAGGT | CGCAGGCGCGUCUUCCAGGU | BE | CGC > CAC | DNA BINDING |
| | R98 | GCACCGCAGGCGCGTCTTCC | GCACCGCAGGCGCGUCUUCC | BE | CGG > CAG | DNA BINDING |
| | K103 | GAACAAGAGCAATGACTTTG | GAACAAGAGCAAUGACUUUG | ABE | AAG > GGG | DNA BINDING |
| PD1 | Exon 1 | CACCTACCTAAGAACCATCC | CACCUACCUAAGAACCAUCC | BE | | KNOCKOUT |
| | STOP | | | | | |
| | Exon 2 | GGGGTTCCAGGGCCTGTCTG | GGGGUUCCAGGGCCUGUCUG | BE | | KNOCKOUT |
| | STOP | | | | | |
| TET2 | H1386 | GACTTGCACAACATGCAGAA | GACUUGCACAACAUGCAGAA | BE | CAC > TAC | DNA BINDING |
| | R1302 | TTGCCAGAAGCAAGATCCCA | UUGCCAGAAGCAAGAUCCCA | ABE | AGA > GGG | DNA BINDING |
| | S1290 | CCATGAACAACCAAAAGAGA | CCAUGAACAACCAAAAGAGA | ABE | TCA > CCA | DNA BINDING |
| SMARCA4 | T353 | TCACCCCCATCCAGAAGCCG | UCACCCCCAUCCAGAAGCCG | BE | ACC > ATT | PHOSPHORYL ATION |
| | S610 | ATCTGGCTGGTCTCGTCCAG | AUCUGGCUGGUCUCGUCCAG | BE | AGC < ATC | PHOSPHORYL ATION |
| | S613 | GATGAGCGACCTCCCGGTGA | GAUGAGCGACCUCCCGGUGA | ABE | AGC > GGC | PHOSPHORYL ATION |
| | S695 | AGACAGCGATGACGTCTCTG | AGACAGCGAUGACGUCUCUG | ABE | AGC > GGC | PHOSPHORYL ATION |
| | S699 | ACGTCTCTGAGGTGGACGCG | ACGUCUCUGAGGUGGACGCG | BE | TCT > TTT | PHOSPHORYL ATION |
| | S1452 | TTAGGGGAGAGTTTCTCGGC | UUAGGGGAGAGUUUCUCGGC | ABE | TCC > CCC | PHOSPHORYL ATION |
| | S1575 | GGAGAGTGAGGAGGAGGAAG | GGAGAGUGAGGAGGAGGAAG | ABE | AGT > GGT | PHOSPHORYL ATION |
| | S1586 | AAGGCTCCGAATCCGAATCT | AAGGCUCCGAAUCCGAAUCU | BE | TCC > TTT | PHOSPHORYL ATION |
| | S1627 | ATCGTCACTCACGACCGGCT | AUCGUCACUCACGACCGGCU | BE | AGT > AAT | PHOSPHORYL ATION |
| | S1631 | TGACAGTGAGGAGGAACAAG | UGACAGUGAGGAGGAACAAG | ABE | AGT > GGT | PHOSPHORYL ATION |
| CDK4 | P173 | CACCCGTGGTTGTTACACTC | CACCCGUGGUUGUUACACUC | BE | CCC > CTT | |
| ZAP70 | S144 | CATCAGCCAGGCCCCGCAGG | CAUCAGCCAGGCCCCGCAGG | ABE | AGC > TGC | PHOSPHORYL ATION |
| | Y292 | GGTGTATCCATCTGAGTTGA | GGUGUAUCCAUCUGAGUUGA | ABE | TAC > CAC | PHOSPHORYL ATION |
| | Y292 | GGGTGTATCCATCTGAGTTG | GGGUGUAUCCAUCUGAGUUG | ABE | TAC > CAC | PHOSPHORYL ATION |
| | R360 | GCGCAAGAAGCAGATCGACG | GCGCAAGAAGCAGAUCGACG | BE | CGC > TGC | Hypermorphic |
| | | | | | | activity |
| | Y598 | TTACTACAGCCTGGCCAGCA | UUACUACAGCCUGGCCAGCA | ABE | TAC > TGC | PHOSPHORYL ATION |

The cytidine and adenosine deaminase nucleobase editors used in this invention can act on DNA, including single stranded DNA. Methods of using them to generate modifications in target nucleobase sequences in immune cells are presented.

In certain embodiments, the fusion proteins provided herein comprise one or more features that improve the base editing activity of the fusion proteins. For example, any of the fusion proteins provided herein may comprise a Cas9 domain that has reduced nuclease activity. In some embodiments, any of the fusion proteins provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-methylated) strand opposite the targeted nucleobase. Mutation of the catalytic residue (e.g., D10 to A10) prevents cleavage of the edited strand containing the targeted A residue. Such Cas9 variants can generate a single-strand DNA break (nick) at a specific location based on the gRNA-defined target sequence, leading to repair of the non-edited strand, ultimately resulting in a nucleobase change on the non-edited strand.

### Adenosine Deaminases

In some embodiments, the fusion proteins of the invention comprise an adenosine deaminase domain. In some embodiments, the adenosine deaminases provided herein are capable of deaminating adenine. In some embodiments, the adenosine deaminases provided herein are capable of deaminating adenine in a deoxyadenosine residue of DNA. The adenosine deaminase may be derived from any suitable organism (e.g., E. coli). In some embodiments, the adenine deaminase is a naturally-occurring adenosine deaminase that includes one or more mutations corresponding to any of the mutations provided herein (e.g., mutations in ecTadA). One of skill in the art will be able to identify the corresponding residue in any homologous protein, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In one embodiment, a fusion protein of the invention comprises a wild-type TadA is linked to TadA7.10, which is linked to Cas9 nickase. In particular embodiments, the fusion proteins comprise a single TadA7.10 domain (e.g., provided as a monomer). In other embodiments, the ABE7.10 editor comprises TadA7.10 and TadA(wt), which are capable of forming heterodimers. The relevant sequences follow:

TadA (wt):
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
RRQEIKAQKKAQSSTD
TadA7.10:
SEVEFSHEYWMRHALTLAKRARDEREVPVGAVLVLNNRVIGEGWNRAIGL
HDPTAHAEIMALRQGGLVMQNYRLIDATLYVTFEPCVMCAGAMIHSRIGR
VVFGVRNAKTGAAGSLMDVLHYPGMNHRVEITEGILADECAALLCYFFRM
PRQVFNAQKKAQSSTD

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

In some embodiments, the adenosine deaminase comprises a D108X mutation in the TadA reference sequence, or a corresponding mutation in another adenosine deaminase, where X indicates any amino acid other than the corresponding amino acid in the wild-type adenosine deaminase. In some embodiments, the adenosine deaminase comprises a D108G, D108N, D108V, D108A, or D108Y mutation in TadA reference sequence, or a corresponding mutation in another adenosine deaminase. It should be appreciated, however, that additional deaminases may similarly be aligned to identify homologous amino acid residues that can be mutated as provided herein.

In some embodiments, the adenosine deaminase comprises an A106X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A106V mutation in TadA reference sequence, or a corresponding mutation in another adenosine deaminase.

In some embodiments, the adenosine deaminase comprises a E155X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a E155D, E155G, or E155V mutation in TadA reference sequence, or a corresponding mutation in another adenosine deaminase.

In some embodiments, the adenosine deaminase comprises a D147X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a D147Y, mutation in TadA reference sequence, or a corresponding mutation in another adenosine deaminase.

It should be appreciated that any of the mutations provided herein (e.g., based on the ecTadA amino acid sequence of TadA reference sequence) may be introduced into other adenosine deaminases, such as S. aureus TadA (saTadA), or other adenosine deaminases (e.g., bacterial adenosine deaminases). It would be apparent to the skilled artisan how to are homologous to the mutated residues in ecTadA. Thus, any of the mutations identified in ecTadA may be made in other adenosine deaminases that have homologous amino acid residues. It should also be appreciated that any of the mutations provided herein may be made individually or in any combination in ecTadA or another adenosine deaminase. For example, an adenosine deaminase may contain a D108N, a A106V, a E155V, and/or a D147Y mutation in TadA reference sequence, or a corresponding mutation in another adenosine deaminase. In some embodiments, an adenosine deaminase comprises the following group of mutations (groups of mutations are separated by a “;”) in TadA reference sequence, or corresponding mutations in another adenosine deaminase: D108N and A106V; D108N and E155V; D108N and D147Y; A106V and E155V; A106V and D147Y; E155V and D147Y; D108N, A106V, and E55V; D108N, A106V, and D147Y; D108N, E55V, and D147Y; A106V, E55V, and D147Y; and D108N, A106V, E55V, and D147Y. It should be appreciated, however, that any combination of corresponding mutations provided herein may be made in an adenosine deaminase (e.g., ecTadA).

In some embodiments, the adenosine deaminase comprises one or more of a H8X, T17X, L18X, W23X, L34X, W45X, R51X, A56X, E59X, E85X, M94X, I95X, V102X, F104X, A106X, R107X, D108X, K10X, M118X, N127X, A138X, F149X, M151X, R153X, Q154X, I156X, and/or K157X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H8Y, T17S, L18E, W23L, L34S, W45L, R51H, A56E, or A56S, E59G, E85K, or E85G, M94L, 1951, V102A, F104L, A106V, R107C, or R107H, or R107P, D108G, or D108N, or D108V, or D108A, or D108Y, Kl 101, Ml 18K, N127S, A138V, F149Y, M151V, R153C, Q154L, I156D, and/or K157R mutation in TadA reference sequence, or one or more corresponding mutations in another adenosine deaminase.

In some embodiments, the adenosine deaminase comprises one or more of H8X, D108X, and/or N127X mutation in TadA reference sequence, or one or more corresponding mutations in another adenosine deaminase, where X indicates the presence of any amino acid. In some embodiments, the adenosine deaminase comprises one or more of a H8Y, D108N, and/or N127S mutation in TadA reference sequence, or one or more corresponding mutations in another adenosine deaminase.

In some embodiments, the adenosine deaminase comprises one or more of H8X, R26X, M61X, L68X, M70X, A106X, D108X, A109X, N127X, D147X, R152X, Q154X, E155X, K161X, Q163X, and/or T166X mutation in TadA reference sequence, or one or more corresponding mutations in another adenosine deaminase, where X indicates the presence of any amino acid other than the corresponding amino acid in the wild-type adenosine deaminase. In some embodiments, the adenosine deaminase comprises one or more of H8Y, R26W, M611, L68Q, M70V, A106T, D108N, A109T, N127S, D147Y, R152C, Q154H or Q154R, E155G or E155V or E155D, K161Q, Q163H, and/or T166P mutation in TadA reference sequence, or one or more corresponding mutations in another adenosine deaminase.

In some embodiments, the adenosine deaminase comprises one, two, three, four, five, or six mutations selected from the group consisting of H8X, D108X, N127X, D147X, R152X, and Q154X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, M61X, M70X, D108X, N127X, Q154X, E155X, and Q163X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, N127X, E155X, and T166X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of H8X, A106X, and D108X, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, or seven mutations selected from the group consisting of H8X, R126X, L68X, D108X, N127X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, D108X, A109X, N127X, and E155X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, or six mutations selected from the group consisting of H8Y, D108N, N127S, D147Y, R152C, and Q154H in TadA reference sequence, or a corresponding mutation or mutations in another adenosine deaminase. In some embodiments, the adenosine deaminase comprises one, two, three, four, five, six, seven, or eight mutations selected from the group consisting of H8Y, M611, M70V, D108N, N127S, Q154R, E155G, and Q163H in TadA reference sequence, or a corresponding mutation or mutations in another adenosine deaminase. In some embodiments, the adenosine deaminase comprises one, two, three, four, or five mutations selected from the group consisting of H8Y, D108N, N127S, E155V, and T166P in TadA reference sequence, or a corresponding mutation or mutations in another adenosine deaminase. In some embodiments, the adenosine deaminase comprises one, two, three, four, five, or six mutations selected from the group consisting of H8Y, A106T, D108N, N127S, E155D, and K161Q in TadA reference sequence, or a corresponding mutation or mutations in another adenosine deaminase. In some embodiments, the adenosine deaminase comprises one, two, three, four, five, six, or seven mutations selected from the group consisting of H8Y, R126W, L68Q, D108N, N127S, D147Y, and E155V in TadA reference sequence, or a corresponding mutation or mutations in another adenosine deaminase. In some embodiments, the adenosine deaminase comprises one, two, three, four, or five mutations selected from the group consisting of H8Y, D108N, A109T, N127S, and E155G in TadA reference sequence, or a corresponding mutation or mutations in another adenosine deaminase.

In some embodiments, the adenosine deaminase comprises one or more of the or one or more corresponding mutations in another adenosine deaminase. In some embodiments, the adenosine deaminase comprises a D108N, D108G, or D108V mutation in TadA reference sequence, or corresponding mutations in another adenosine deaminase. In some embodiments, the adenosine deaminase comprises a A106V and D108N mutation in TadA reference sequence, or corresponding mutations in another adenosine deaminase. In some embodiments, the adenosine deaminase comprises R107C and D108N mutations in TadA reference sequence, or corresponding mutations in another adenosine deaminase. In some embodiments, the adenosine deaminase comprises a H8Y, D108N, N127S, D147Y, and Q154H mutation in TadA reference sequence, or corresponding mutations in another adenosine deaminase. In some embodiments, the adenosine deaminase comprises a H8Y, R24W, D108N, N127S, D147Y, and E155V mutation in TadA reference sequence, or corresponding mutations in another adenosine deaminase. In some embodiments, the adenosine deaminase comprises a D108N, D147Y, and E155V mutation in TadA reference sequence, or corresponding mutations in another adenosine deaminase. In some embodiments, the adenosine deaminase comprises a H8Y, D108N, and N127S mutation in TadA reference sequence, or corresponding mutations in another adenosine deaminase. In some embodiments, the adenosine deaminase comprises a A106V, D108N, D147Y, and E155V mutation in TadA reference sequence, or corresponding mutations in another adenosine deaminase.

In some embodiments, the adenosine deaminase comprises one or more of S2X, H8X, 149X, L84X, H123X, N127X, I156X, and/or K160X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of S2A, H8Y, 149F, L84F, H123Y, N127S, I156F, and/or K160S mutation in TadA reference sequence, or one or more corresponding mutations in another adenosine deaminase.

In some embodiments, the adenosine deaminase comprises an L84X mutation adenosine deaminase, where X indicates any amino acid other than the corresponding amino acid in the wild-type adenosine deaminase. In some embodiments, the adenosine deaminase comprises an L84F mutation in TadA reference sequence, or a corresponding mutation in another adenosine deaminase.

In some embodiments, the adenosine deaminase comprises an H123X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H123Y mutation in TadA reference sequence, or a corresponding mutation in another adenosine deaminase.

In some embodiments, the adenosine deaminase comprises an 1157X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an I157F mutation in TadA reference sequence, or a corresponding mutation in another adenosine deaminase.

In some embodiments, the adenosine deaminase comprises one, two, three, four, five, six, or seven mutations selected from the group consisting of L84X, A106X, D108X, H123X, D147X, E155X, and I156X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of S2X, I49X, A106X, D108X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, A106X, D108X, N127X, and K160X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in TadA reference sequence, or a corresponding mutation or mutations in another adenosine deaminase. In some embodiments, the adenosine deaminase comprises one, two, three, four, five, or six mutations selected from the group consisting of S2A, I49F, A106V, D108N, D147Y, and E155V in TadA reference sequence.

In some embodiments, the adenosine deaminase comprises one, two, three, four, or five mutations selected from the group consisting of H8Y, A106T, D108N, N127S, and K160S in TadA reference sequence, or a corresponding mutation or mutations in another adenosine deaminase.

In some embodiments, the adenosine deaminase comprises one or more of a E25X, R26X, R107X, A142X, and/or A143X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of E25M, E25D, E25A, E25R, E25V, E25S, E25Y, R26G, R26N, R26Q, R26C, R26L, R26K, R107P, R07K, R107A, R107N, R107W, R107H, R107S, A142N, A142D, A142G, A143D, A143G, A143E, A143L, A143W, A143M, A143S, A143Q, and/or A143R mutation in TadA reference sequence, or one or more corresponding mutations in another adenosine deaminase. In some embodiments, the adenosine deaminase comprises one or more of the mutations described herein corresponding to TadA reference sequence, or one or more corresponding mutations in another adenosine deaminase.

In some embodiments, the adenosine deaminase comprises an E25X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an E25M, E25D, E25A, E25R, E25V, E25S, or E25Y mutation in TadA reference sequence, or a corresponding mutation in another adenosine deaminase.

In some embodiments, the adenosine deaminase comprises an R26X mutation in TadA reference sequence, or a corresponding mutation in another adenosine deaminase, where X indicates any amino acid other than the corresponding amino acid in the wild-type adenosine deaminase. In some embodiments, the adenosine deaminase comprises R26G, R26N, R26Q, R26C, R26L, or R26K mutation in TadA reference sequence, or a corresponding mutation in another adenosine deaminase.

In some embodiments, the adenosine deaminase comprises an R107X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R107P, R07K, R107A, R107N, R107W, R107H, or R107S mutation in TadA reference sequence, or a corresponding mutation in another adenosine deaminase.

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A142N, A142D, A142G, mutation in TadA reference sequence, or a corresponding mutation in another adenosine deaminase.

In some embodiments, the adenosine deaminase comprises an A143X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A143D, A143G, A143E, A143L, A143W, A143M, A143S, A143Q, and/or A143R mutation in TadA reference sequence, or a corresponding mutation in another adenosine deaminase.

In some embodiments, the adenosine deaminase comprises one or more of a H36X, N37X, P48X, 149X, R51X, M70X, N72X, D77X, E134X, S146X, Q154X, K157X, and/or K161X mutation in TADA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H36L, N37T, N37S, P48T, P48L, 149V, R51H, R51L, M70L, N72S, D77G, E134G, S146R, S146C, Q154H, K157N, and/or K161T mutation in TadA reference sequence, or one or more corresponding mutations in another adenosine deaminase.

In some embodiments, the adenosine deaminase comprises an H36X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H36L mutation in TadA reference sequence, or a corresponding mutation in another adenosine deaminase.

In some embodiments, the adenosine deaminase comprises an N37X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an N37T or N37S mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an P48T or P48L mutation in TadA reference sequence, or a corresponding mutation in another adenosine deaminase.

In some embodiments, the adenosine deaminase comprises an R51X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R51H or R51L mutation in TadA reference sequence, or a corresponding mutation in another adenosine deaminase.

In some embodiments, the adenosine deaminase comprises an S146X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an S146R or S146C mutation in TadA reference sequence, or a corresponding mutation in another adenosine deaminase.

In some embodiments, the adenosine deaminase comprises an K157X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a K157N mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a P48S, P48T, or P48A mutation in TadA reference sequence, or a corresponding mutation in another adenosine deaminase.

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a A142N mutation in TadA reference sequence, or a corresponding mutation in another adenosine deaminase.

In some embodiments, the adenosine deaminase comprises an W23X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a W23R or W23L mutation in TadA reference sequence, or a corresponding mutation in another adenosine deaminase.

In some embodiments, the adenosine deaminase comprises an R152X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a R152P or R52H mutation in TadA reference sequence, or a corresponding mutation in another adenosine deaminase.

In one embodiment, the adenosine deaminase may comprise the mutations H36L, R51L, L84F, A106V, D108N, H123Y, S146C, D147Y, E155V, I156F, and K157N. In some embodiments, the adenosine deaminase comprises the following combination of mutations relative to TadA reference sequence, where each mutation of a combination is separated by a “_” and each combination of mutations is between parentheses: (A106V_D108N),

### (R107C_D108N),

### (H8Y_D108N_S127S_D147Y_Q154H), (H8Y_R24W_D108N_N127S_D147Y_E155V),

### (D108N_D147Y_E155V), (H8Y_D108N_S127S), (H8Y_D108N_N127S_D147Y_Q154H),

### (A106V_D108N_D147Y_E155V) (D108Q_D147Y_E155V) (D108M_D147Y_E155V),

### (D108L_D147Y_E155V), (D108K_D147Y_E155V), (D1081_D147Y_E155V),

### (D108F_D147Y_E155V), (A106V_D108N_D147Y), (A106V_D108M_D147Y_E155V),

(E59A_A106V_D108N_D147Y_E155V), (E59A cat dead_A106V_D108N_D147Y_E155V),

### (L84F_A106V_D108N_H123Y_D147Y_E155V_I156Y),

### (L84F_A106V_D108N_H123Y_D147Y_E155V_I156F), (D103A_D014N),

### (G22P_D103A_D104N), (G22P_D103A_D104N_S138A), (D103A_D104N_S138A),

### (R26G_L84F_A106V_R107H_D108N_H123Y_A 142N_A143D_D147Y_E155V_I156F),

### (E25G_R26G_L84F_A106V_R107H_D108N_H123Y_A142N_A143D_D147Y_E155V_I156F), (E25D_R26G_L84F_A106V_R107K_D108N_H123Y_A142N_A143G_D147Y_E155V_I156F), (R26Q_L84F_A106V_D108N_H123Y_A142N_D147Y_E155V_I156F),

### (E25M_R26G_L84F_A106V_R107P_D108N_H123Y_A142N_A143D_D147Y_E155V_I156F), (R26C_L84F_A106V_R107H_D108N_H123Y_A142N_D147Y_E155V_I156F),

### (L84F_A106V_D108N_H123Y_A142N_A143L_D147Y_E155V_I156F),

### (R26G_L84F_A106V_D108N_H123Y_A142N_D147Y_E155V_I156F),

### (E25A_R26G_L84F_A106V_R107N_D108N_H123Y_A142N_A143E_D147Y_E155V_I156F),

### (R26G_L84F_A106V_R107H_D108N_H123Y_A 142N_A143D_D147Y_E155V_I156F),

### (A106V_D108N_A142N_D147Y_E155V), (R26G_A106V_D108N_A142N_D147Y_E155V),

### (E25D_R26G_A106V_R107K_D108N_A142N_A143G_D147Y_E155V),

### (R26G_A106V_D108N_R107H_A142N_A143D_D147Y_E155V),

### (E25D_R26G_A106V_D108N_A142N_D147Y_E155V),

### (A106V_R107K_D108N_A142N_D147Y_E155V),

### (A106V_D108N_A142N_A143G_D147Y_E155V),

### (A106V_D108N_A142N_A143L_D147Y_E155V),

### (H36L_R51L_L84F_A106V_D108N_H123Y_S146C_D147Y_E155V_I156F_K157N),

### (N37T_P48T_M70L_L84F_A106V_D108N_H123Y_D147Y_I49V_E155V_I156F),

### (N37S_L84F_A106V_D108N_H123Y_D147Y_E155V_I156F_K161T),

### (H36L_L84F_A 106V_D108N_H123Y_D147Y_Q154H_E155V_I156F),

### (N72S_L84F_A106V_D108N_H123Y_S146R_D147Y_E155V_I156F),

### (H36L_P48L_L84F_A106V_D108N_H123Y_E134G_D147Y_E155V_I156F_K157N),

### (H36L_L84F_A 106V_D108N_H123Y_S146C_D147Y_E155V_I156F),

### (L84F_A106V_D108N_H123Y_S146R_D147Y_E155V_I156F_K161T),

### (N37S_R51H_D77G_L84F_A106V_D108N_H123Y_D147Y_E155V_I156F),

### (R51L_L84F_A106V_D108N_H123Y_D147Y_E155V_I156F_K157N),

### (D24G_Q71R_L84F_H96L_A106V_D108N_H123Y_D147Y_E155V_I156F_K160E),

### (H36L_G67V_L84F_A106V_D108N_H123Y_S146T_D147Y_E155V_I156F),

### (Q71L_L84F_A 106V_D108N_H123Y_L137M_A143E_D147Y_E155V_I156F),

### (E25G_L84F_A 106V_D108N_H123Y_D147Y_E155V_I156F_Q159L),

### (L84F_A91T_F104I_A106V_D108N_H123Y_D147Y_E155V_I156F),

### (N72D_L84F_A106V_D108N_H123Y_G125A_D147Y_E155V_I156F),

### (P48S_L84F_S97C_A106V_D108N_H123Y_D147Y_E155V_I156F),

### (W23G_L84F_A106V_D108N_H123Y_D147Y_E155V_I156F),

### (D24G_P48L_Q71R_L84F_A106V_D108N_H123Y_D147Y_E155V_I156F_Q159L),

### (L84F_A106V_D108N_H123Y_A142N_D147Y_E155V_I156F),

### (H36L_R51L_L84F_A106V_D108N_H123Y_A142N_S146C_D147Y_E155V_I156F_K157N),

### (N37S_L84F_A106V_D108N_H123Y_A142N_D147Y_E155V_I156F_K161T),

### (L84F_A106V_D108N_D147Y_E155V_I156F),

### (R51L_L84F_A106V_D108N_H123Y_S146C_D147Y_E155V_I156F_K157N_K161T),

### (L84F_A106V_D108N_H123Y_S146C_D147Y_E155V_I156F_K161T),

### (L84F_A106V_D108N_H123Y_S146C_D147Y_E155V_I156F_K157N_K160E_K161T),

### (L84F_A106V_D108N_H123Y_S146C_D147Y_E155V_I156F_K157N_K160E),

### (R74Q_L84F_A106V_D108N_H123Y_D147Y_E155V_I156F),

### (R74A_L84F_A106V_D108N_H123Y_D147Y_E155V_I156F),

### (L84F_A106V_D108N_H123Y_D147Y_E155V_I156F),

### (R74Q_L84F_A106V_D108N_H123Y_D147Y_E155V_I156F),

### (L84F_R98Q_A106V_D108N_H123Y_D147Y_E155V_I156F),

### (L84F_A106V_D108N_H123Y_R129Q_D147Y_E155V_I156F),

### (P48S_L84F_A106V_D108N_H123Y_A142N_D147Y_E155V_I156F), (P48S_A142N),

### (P48T_I49V_L84F_A106V_D108N_H123Y_A142N_D147Y_E155V_I156F_L157N),

### (P48T_I49V_A142N), (H36L_P48S_R51L_L84F_A106V_D108N_H123Y_S146C_D147Y_E155V_I156F_K157N),

### (H36L_P48S_R51L_L84F_A106V_D108N_H123Y_S146C_A142N_D147Y_E155V_I156F

### (H36L_P48T_I49V_R51L_L84F_A106V_D108N_H123Y_S146C_D147Y_E155V_I156F_K157N), (H36L_P48T_I49V_R51L_L84F_A106V_D108N_H123Y_A142N_S146C_D147 Y_E155V_I156F_K157N),

### (H36L_P48A_R51L_L84F_A106V_D108N_H123Y_S146C_D147Y_E155V_I156F_K157N),

### (H36L_P48A_R51L_L84F_A106V_D108N_H123Y_A142N_S146C_D147Y_E155V_I156F_K157N),

### (H36L_P48A_R51L_L84F_A106V_D108N_H123Y_S146C_A142N_D147Y_E155V_I156F_K157N),

### (W23L_H36L_P48A_R51L_L84F_A106V_D108N_H123Y_S146C_D147Y_E155V_I156F_K157N),

### (W23R_H36L_P48A_R51L_L84F_A106V_D108N_H123Y_S146C_D147Y_E155V_I156F_K157N),

### (W23L_H36L_P48A_R51L_L84F_A106V_D108N_H123Y_S146R_D147Y_E155V_I156F_K161T),

### (H36L_P48A_R51L_L84F_A106V_D108N_H123Y_S146C_D147Y_R152H_E155V_I156F_K157N),

### (H36L_P48A_R51L_L84F_A106V_D108N_H123Y_S146C_D147Y_R152P_E155V_I156F_K157N),

### (W23L_H36L_P48A_R51L_L84F_A106V_D108N_H123Y_S146C_D147Y_R152P_E155V_I156F_K157N),

### (W23L_H36L_P48A_R51L_L84F_A106V_D108N_H123Y_A142A_S146C_D147Y_E155V_I156F_K157N),

### (W23L_H36L_P48A_R51L_L84F_A106V_D108N_H123Y_A142A_S146C_D147Y_R152P_E155V_I156F_K157N),

### (W23L_H36L_P48A_R51L_L84F_A106V_D108N_H123Y_S146R_D147Y_E155V_I156F_K161T),

### (W23R_H36L_P48A_R51L_L84F_A106V_D108N_H123Y_S146C_D147Y_R152P_E155V_I156F_K157N),

### (H36L_P48A_R51L_L84F_A106V_D108N_H123Y_A142N_S146C_D147Y_R152P_E155V_I156F_K157N).

### Cytidine Deaminase

In addition to adenosine deaminase, the fusion proteins of the invention comprise one or more cytidine deaminases. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

The disclosure provides any deaminase domains with a certain percent identity plus any of the mutations or combinations thereof described herein. In some embodiments, the cytidine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the cytidine deaminases provided herein. In some embodiments, the cytidine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

A fusion protein of the invention second protein comprises two or more nucleic acid editing domains. In some embodiments, the nucleic acid editing domain can catalyze a C to U base change. In some embodiments, the nucleic acid editing domain is a deaminase domain. In some embodiments, the deaminase is a cytidine deaminase. In some embodiments, the deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3 A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the deaminase is a vertebrate deaminase. In some embodiments, the deaminase is an invertebrate deaminase. In some embodiments, the deaminase is a human, chimpanzee, gorilla, monkey, cow, dog, rat, or mouse deaminase. In some embodiments, the deaminase is a human deaminase. In some embodiments, the deaminase is a rat deaminase, e.g., rAPOBEC1. In some embodiments, the deaminase is a Petromyzon marinus cytidine deaminase 1 (pmCDA1). In some embodiments, the deminase is a human APOBEC3G. In some embodiments, the deaminase is a fragment of the human APOBEC3G. In some embodiments, the deaminase is a human APOBEC3G variant comprising a D316R D317R mutation. In some embodiments, the deaminase is a fragment of the human APOBEC3G and comprising mutations corresponding to the D316R D317R mutations. In some embodiments, the nucleic acid editing domain is at least 80%, at least 85%, at least 90%, at least 92%, at least 95%, at least 96%, at least 97%, at least 98%, at least 99%), or at least 99.5% identical to the deaminase domain of any deaminase described herein.

In certain embodiments, the fusion proteins provided herein comprise one or more features that improve the base editing activity of the fusion proteins. For example, any of the fusion proteins provided herein may comprise a Cas9 domain that has reduced nuclease activity. In some embodiments, any of the fusion proteins provided herein may have a Cas9 domain that does not have nuclease activity (dCas9), or a Cas9 domain that cuts one strand of a duplexed DNA molecule, referred to as a Cas9 nickase (nCas9).

### Cas9 Domains of Nucleobase Editors

In some aspects, a nucleic acid programmable DNA binding protein (napDNAbp) is selected from the group consisting of Cas9, CasX, CasY, Cpf1, Cas12b/C2c1, and Cas12c/C2c3, or active fragments thereof. In another embodiment, the napDNAbp domain comprises a catalytic domain capable of cleaving the reverse complement strand of the nucleic acid sequence. In another embodiment, the napDNAbp domain does not comprise a catalytic domain capable of cleaving the nucleic acid sequence. In another embodiment, the Cas9 is dCas9 or nCas9. In another embodiment, the napDNAbp comprises a nucleobase editor.

In some embodiments, a nucleic acid programmable DNA binding protein (napDNAbp) is a Cas9 domain. Non-limiting, exemplary Cas9 domains are provided herein. The Cas9 domain may be a nuclease active Cas9 domain, a nuclease inactive Cas9 domain (a nuclease dead Cas9, or dCas9), or a Cas9 nickase (nCas9). In some embodiments, the Cas9 domain is a nuclease active domain. For example, the Cas9 domain may be a Cas9 domain that cuts both strands of a duplexed nucleic acid (e.g., both strands of a duplexed DNA molecule). In some embodiments, the Cas9 domain comprises any one of the amino acid sequences as set forth herein. In some embodiments the Cas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth herein. In some embodiments, the Cas9 domain comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared to any one of the amino acid sequences set forth herein. In some embodiments, the Cas9 domain comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth herein.

In some embodiments, the Cas9 domain is a nuclease-inactive Cas9 domain (dCas9). For example, the dCas9 domain may bind to a duplexed nucleic acid molecule (e.g., via a gRNA molecule) without cleaving either strand of the duplexed nucleic acid molecule. In some embodiments, the nuclease-inactive dCas9 domain comprises a D10X mutation and a H840X mutation of the amino acid sequence set forth herein, or a corresponding mutation in any of the amino acid sequences provided herein, wherein X is any amino acid change. In some embodiments, the nuclease-inactive dCas9 domain comprises a D10A mutation and a H840A mutation of the amino acid sequence set forth herein, or a corresponding mutation in any of the amino acid sequences provided herein. As one example, a nuclease-inactive Cas9 domain comprises the amino acid sequence set forth in Cloning vector pPlatTET-gRNA2 (Accession No. BAV54124).

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

(see, e.g., Qi et al., “Repurposing CRISPR as an RNA-guided platform for sequence-specific control of gene expression.” Cell. 2013; 152(5):1173-83, the entire contents of which are incorporated herein by reference).

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference). In some embodiments the dCas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the dCas9 domains provided herein. In some embodiments, the Cas9 domain comprises an amino acid sequences that has 1, 2, 3, 4, 5,6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared to any one of the amino acid sequences set forth herein. In some embodiments, the Cas9 domain comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth herein.

In some embodiments, the Cas9 domain is a Cas9 nickase. The Cas9 nickase may be a Cas9 protein that is capable of cleaving only one strand of a duplexed nucleic acid molecule (e.g., a duplexed DNA molecule). In some embodiments the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase comprises a D10A mutation and has a histidine at position 840. In some embodiments the Cas9 nickase cleaves the non-target, non-base-edited strand of a duplexed nucleic acid molecule, meaning that the Cas9 nickase cleaves the strand that is not base paired to a gRNA (e.g., an sgRNA) that is bound to the Cas9. In some embodiments, a Cas9 nickase comprises an H840A mutation and has an aspartic acid residue at position 10, or a corresponding mutation. In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field, and are within the scope of this disclosure.

Cas9 Domains with Reduced PAM Exclusivity

Some aspects of the disclosure provide Cas9 domains that have different PAM specificities. In one particular embodiment, the invention features nucleobase editor fusion proteins that comprise an nCas9 domain and a dCas9 domain, where each of the Cas9 domains has a different PAM specificity. Typically, Cas9 proteins, such as Cas9 from S. pyogenes (spCas9), require a canonical NGG PAM sequence to bind a particular nucleic acid region, where the “N” in “NGG” is adenosine (A), thymidine (T), or cytosine (C), and the G is guanosine. This may limit the ability to edit desired bases within a genome. In some embodiments, the base editing fusion proteins provided herein may need to be placed at a precise location, for example a region comprising a target base that is upstream of the PAM. See e.g., Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein may contain a Cas9 domain that can bind a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference. Several PAM variants are described at Table 3 below:

TABLE 3
Cas9 proteins and corresponding PAM sequences

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| Cpfl | 5′(TTTV) |

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises a N579A mutation, or a corresponding mutation in any of the amino acid sequences provided herein.

In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT PAM sequence. In some embodiments, the SaCas9 domain comprises one or more of a E781X, a N967X, and a R1014X mutation, or a corresponding mutation in any of the amino acid sequences provided herein, wherein X is any amino acid. In some embodiments, the SaCas9 domain comprises one or more of a E781K, a N967K, and a R1014H mutation, or one or more corresponding mutation in any of the amino acid sequences provided herein. In some embodiments, the SaCas9 domain comprises a E781K, a N967K, or a R1014H mutation, or corresponding mutations in any of the amino acid sequences provided herein.

### Exemplary SaCas9 Sequence

KRNYILGLDIGITSVGYGIIDYETRDVIDAGVRLFKEA
NVENNEGRRSKRGARRLKRRRRHRIQRVKKLLFDYNLL
TDHSELSGINPYEARVKGLSQKLSEEEFSAALLHLAKRR
GVHNVNEVEEDTGNELSTKEQISRNSKALEEKYVAELQL
ERLKKDGEVRGSINRFKTSDYVKEAKQLLKVQKAYHQLD
QSFIDTYIDLLETRRTYYEGPGEGSPFGWKDIKEWYEML
MGHCTYFPEELRSVKYAYNADLYNALNDLNNLVITRDEN
EKLEYYEKFQIIENVFKQKKKPTLKQIAKEILVNEEDIK
GYRVTSTGKPEFTNLKVYHDIKDITARKEIIENAELLDQ
IAKILTIYQSSEDIQEELTNLNSELTQEEIEQISNLKGY
TGTHNLSLKAINLILDELWHTNDNQIAIFNRLKLVPKKV
DLSQQKEIPTTLVDDFILSPVVKRSFIQSIKVINAIIK
KYGLPNDIIIELAREKNSKDAQKMINEMQKRNRQTNERI
EEIIRTTGKENAKYLIEKIKLHDMQEGKCLYSLEAIPLE
DLLNNPFNYEVDHIIPRSVSFDNSFNNKVLVKQEENSKK
GNRTPFQYLSSSDSKISYETFKKHILNLAKGKGRISKTK
KEYLLEERDINRFSVQKDFINRNLVDTRYATRGLMNLLR
SYFRVNNLDVKVKSINGGFTSFLRRKWKFKKERNKG
YKHHAEDALIIANADFIFKEWKKLDKAKKVMENQMFEEK
QAESMPEIETEQEYKEIFITPHQIKHIKDFKDYKYSHRV
DKKPNRELINDTLYSTRKDDKGNTLIVNNLNGLYDK
DNDKLKKLINKSPEKLLMYHHDPQTYQKLKLIMEQYGDE
KNPLYKYYEETGNYLTKYSKKDNGPVIKKIKYYGNKLNA
HLDITDDYPNSRNKVVKLSLKPYRFDVYLDNGVYKFVTV
KNLDVIKKENYYEVNSKCYEEAKKLKKISNQAEFIASFY
NNDLIKINGELYRVIGVNNDLLNRIEVNMIDITYREYLE
NMNDKRPPRIIKTIASKTQSIKKYSTDILGNLYEVKSKK
HPQIIKKG

Residue N579 above, which is underlined and in bold, may be mutated (e.g., to a A579) to yield a SaCas9 nickase.

### Exemplary SaCas9n Sequence

KRNYILGLDIGITSVGYGIIDYETRDVIDAGVRLFKEA
NVENNEGRRSKRGARRLKRRRRHRIQRVKKLLFDYNLL
TDHSELSGINPYEARVKGLSQKLSEEEFSAALLHLAKRR
GVHNVNEVEEDTGNELSTKEQISRNSKALEEKYVAELQL
ERLKKDGEVRGSINRFKTSDYVKEAKQLLKVQKAYHQLD
QSFIDTYIDLLETRRTYYEGPGEGSPFGWKDIKEWYEML
MGHCTYFPEELRSVKYAYNADLYNALNDLNNLVITRDEN
EKLEYYEKFQIIENVFKQKKKPTLKQIAKEILVNEEDIK
GYRVTSTGKPEFTNLKVYHDIKDITARKEIIENAELLDQ
IAKILTIYQSSEDIQEELTNLNSELTQEEIEQISNLKGY
TGTHNLSLKAINLILDELWHTNDNQIAIFNRLKLVPKKV
DLSQQKEIPTTLVDDFILSPVVKRSFIQSIKVINAIIK
KYGLPNDIIIELAREKNSKDAQKMINEMQKRNRQTNERI
EEIIRTTGKENAKYLIEKIKLHDMQEGKCLYSLEAIPLE
DLLNNPFNYEVDHIIPRSVSFDNSFNNKVLVKQEEASKK
GNRTPFQYLSSSDSKISYETFKKHILNLAKGKGRISKTK
KEYLLEERDINRFSVQKDFINRNLVDTRYATRGLMNLLR
SYFRVNNLDVKVKSINGGFTSFLRRKWKFKKERNKGYKH
HAEDALIIANADFIFKEWKKLDKAKKVMENQMFEEKQAE
SMPEIETEQEYKEIFITPHQIKHIKDFKDYKYSHRVDKK
PNRELINDTLYSTRKDDKGNTLIVNNLNGLYDKDNDKLK
KLINKSPEKLLMYHHDPQTYQKLKLIMEQYGDEKNPLYK
YYEETGNYLTKYSKKDNGPVIKKIKYYGNKLNAHLDITD
DYPNSRNKVVKLSLKPYRFDVYLDNGVYKFVTVKNLDVI
KKENYYEVNSKCYEEAKKLKKISNQAEFIASFYNNDLIK
INGELYRVIGVNNDLLNRIEVNMIDITYREYLENMNDKR
PPRIIKTIASKTQSIKKYSTDILGNLYEVKSKKHPQIIKKG

Residue A579 above, which can be mutated from N579 to yield a SaCas9 nickase, is underlined and in bold.

### Exemplary SaKKH Cas9

KRNYILGLDIGITSVGYGIIDYETRDVIDAGVRLFKEANVENNEGRRS
KRGARRLKRRRRHRIQRVKKLLFDYNLLTDHSELSGINPYEARVKGLS
QKLSEEEFSAALLHLAKRRGVHNVNEVEEDTGNELSTKEQISRNSKAL
EEKYVAELQLERLKKDGEVRGSINRFKTSDYVKEAKQLLKVQKAYHQL
DQSFIDTYIDLLETRRTYYEGPGEGSPFGWKDIKEWYEMLMGHCTYFP
EELRSVKYAYNADLYNALNDLNNLVITRDENEKLEYYEKFQIIENVFK
QKKKPTLKQIAKEILVNEEDIKGYRVTSTGKPEFTNLKVYHDIKDITA
RKEIIENAELLDQIAKILTIYQSSEDIQEELTNLNSELTQEEIEQISN
LKGYTGTHNLSLKAINLILDELWHTNDNQIAIFNRLKLVPKKVDLSQQ
KEIPTTLVDDFILSPVVKRSFIQSIKVINAIIKKYGLPNDIIIELARE
KNSKDAQKMINEMQKRNRQTNERIEEIIRTTGKENAKYLIEKIKLHDM
QEGKCLYSLEAIPLEDLLNNPFNYEVDHIIPRSVSFDNSFNNKVLVKQ
EEASKKGNRTPFQYLSSSDSKISYETFKKHILNLAKGKGRISKTKKEY
LLEERDINRFSVQKDFINRNLVDTRYATRGLMNLLRSYFRVNNLDVKV
KSINGGFTSFLRRKWKFKKERNKGYKHHAEDALIIANADFIFKEWKKL
DKAKKVMENQMFEEKQAESMPEIETEQEYKEIFITPHQIKHIKDFKDY
KYSHRVDKKPNRKLINDTLYSTRKDDKGNTLIVNNLNGLYDKDNDKLK
KLINKSPEKLLMYHHDPQTYQKLKLIMEQYGDEKNPLYKYYEETGNYL
TKYSKKDNGPVIKKIKYYGNKLNAHLDITDDYPNSRNKVVKLSLKPYR
FDVYLDNGVYKFVTVKNLDVIKKENYYEVNSKCYEEAKKLKKISNQAE
FIASFYKNDLIKINGELYRVIGVNNDLLNRIEVNMIDITYREYLENMN
DKRPPHIIKTIASKTQSIKKYSTDILGNLYEVKSKKHPQIIKKG.

Residue A579 above, which can be mutated from N579 to yield a SaCas9 nickase, is underlined and in bold. Residues K781, K967, and H1014 above, which can be mutated from E781, N967, and R1014 to yield a SaKKH Cas9 are underlined and in italics.

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises a D9X mutation, or a corresponding mutation in any of the amino acid sequences provided herein, wherein X is any amino acid except for D. In some embodiments, the SpCas9 comprises a D9A mutation, or a corresponding mutation in any of the amino acid sequences provided herein. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having an NGG, a NGA, or a NGCG PAM sequence. In some embodiments, the SpCas9 domain comprises one or more of a D1134X, a R1334X, and a T1336X mutation, or a corresponding mutation in any of the amino acid sequences provided herein, wherein X is any amino acid. In some embodiments, the SpCas9 domain comprises one or more of a D1134E, R1334Q, and T1336R mutation, or a corresponding mutation in any of the amino acid sequences provided herein. In some embodiments, the SpCas9 domain comprises a D1134E, a R1334Q, and a T1336R mutation, or corresponding mutations in any of the amino acid sequences provided herein. In some embodiments, the SpCas9 domain comprises one or more of a D1134X, a R1334X, and a T1336X mutation, or a corresponding mutation in any of the amino acid sequences provided herein, wherein X is any amino acid. In some embodiments, the SpCas9 domain comprises one or more of a D1134V, a R1334Q, and a T1336R mutation, or a corresponding mutation in any of the amino acid sequences provided herein. In some embodiments, the SpCas9 domain comprises a D1134V, a R1334Q, and a T1336R mutation, or corresponding mutations in any of the amino acid sequences provided herein. In some embodiments, the SpCas9 domain comprises one or more of a D1134X, a G1217X, a R1334X, and a T1336X mutation, or a corresponding mutation in any of the amino acid sequences provided herein, wherein X is any amino acid. In some embodiments, the SpCas9 domain comprises one or more of a D1134V, a G1217R, a R1334Q, and a T1336R mutation, or a corresponding mutation in any of the amino acid sequences provided herein. In some embodiments, the SpCas9 domain comprises a D1134V, a G1217R, a R1334Q, and a T1336R mutation, or corresponding mutations in any of the amino acid sequences provided herein.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to a Cas9 polypeptide described herein. In some embodiments, the Cas9 domain of any of the fusion proteins provided herein comprises the amino acid sequence of any Cas9 polypeptide described herein. In some embodiments, the Cas9 domain of any of the fusion proteins provided herein consists of the amino acid sequence of any Cas9 polypeptide described herein.

### Exemplary SpCas9

DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

### Exemplary SpCas9n

DKKYSIGLAIGTNSVGWAVITDEYKVPSKKFKVLGNTDR
HSIKKNLIGALLFDSGETAEATRLKRTARRRYTRRKNRI
CYLQEIFSNEMAKVDDSFFHRLEESFLVEEDKKHERH
PIFGNIVDEVAYHEKYPTIYHLRKKLVDSTDKADLRLIY
LALAHMIKFRGHFLIEGDLNPDNSDVDKLFIQLVQTYN
QLFEENPINASGVDAKAILSARLSKSRRLENLIAQLPGE
KKNGLFGNLIALSLGLTPNFKSNFDLAEDAKLQLSKDTY
DDDLDNLLAQIGDQYADLFLAAKNLSDAILLSDILRV
NTEITKAPLSASMIKRYDEHHQDLTLLKALVRQQLPEKY
KEIFFDQSKNGYAGYIDGGASQEEFYKFIKPILEKMDG
TEELLVKLNREDLLRKQRTFDNGSIPHQIHLGELHAILRR
QEDFYPFLKDNREKIEKILTFRIPYYVGPLARGNSRFAW
MTRKSEETITPWNFEEVVDKGASAQSFIERMTNFDKNL
PNEKVLPKHSLLYEYFTVYNELTKVKYVTEGMRKPAFL
SGEQKKAIVDLLFKTNRKVTVKQLKEDYFKKIECFDS
VEISGVEDRFNASLGTYHDLLKIIKDKDFLDNEENE
DILEDIVLTLTLFEDREMIEERLKTYAHLFDDKVMKQ
LKRRRYTGWGRLSRKLINGIRDKQSGKTILDFLKSDG
FANRNFMQLIHDDSLTFKEDIQKAQVSGQGDSLHEHIA
NLAGSPAIKKGILQTVKVVDELVKVMGRHKPENIVIEM
ARENQTTQKGQKNSRERMKRIEEGIKELGSQILKEHPV
ENTQLQNEKLYLYYLQNGRDMYVDQELDINRLSDYDV
DHIVPQSFLKDDSIDNKVLTRSDKNRGKSDNVPSEE
VVKKMKNYWRQLLNAKLITQRKFDNLTKAERGGLSELD
KAGFIKRQLVETRQITKHVAQILDSRMNTKYDENDKL
IREVKVITLKSKLVSDFRKDFQFYKVREINNYHHAHD
AYLNAVVGTALIKKYPKLESEFVYGDYKVYDVRKMIA
KSEQEIGKATAKYFFYSNIMNFFKTEITLANGEIRKR
PLIETNGETGEIVWDKGRDFATVRKVLSMPQVNIVKK
TEVQTGGFSKESILPKRNSDKLIARKKDWDPKKYGGFD
SPTVAYSVLVVAKVEKGKSKKLKSVKELLGITIMERSS
FEKNPIDFLEAKGYKEVKKDLIIKLPKYSLFELENGR
KRMLASAGELQKGNELALPSKYVNFLYLASHYEKLKG
SPEDNEQKQLFVEQHKHYLDEIIEQISEFSKRVILAD
ANLDKVLSAYNKHRDKPIREQAENIIHLFTLTNLGA
PAAFKYFDTTIDRKRYTSTKEVLDATLIHQSITGLY
ETRIDLSQLGGD

### Exemplary SpEQR Cas9

DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPIN
ASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNL
PNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMG
RHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIRE
VKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQT
GGFSKESILPKRNSDKLIARKKDWDPKKYGGFESPTVAYSVLVVAKVEKG
KSKKLKSVKELLGITIMERSSFEKNPIDFLEAKGYKEVKKDLIIKLPKYS
LFELENGRKRMLASAGELQKGNELALPSKYVNFLYLASHYEKLKGSPEDN
EQKQLFVEQHKHYLDEIIEQISEFSKRVILADANLDKVLSAYNKHRDKPI
REQAENIIHLFTLTNLGAPAAFKYFDTTIDRKQYRSTKEVLDATLIHQSI
TGLYETRIDLSQLGGD

Residues E1134, Q1334, and R1336 above, which can be mutated from D1134, R1334, and T1336 to yield a SpEQR Cas9, are underlined and in bold.

### Exemplary SpVQR Cas9

DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKQYRSTKEVLDATLIHQS
ITGLYETRIDLSQLGGD

Residues V1134, Q1334, and R1336 above, which can be mutated from D1134, R1334, and T1336 to yield a SpVQR Cas9, are underlined and in bold.

### Exemplary SpVRER Cas9

DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKEYRSTKEVLDATLIHQS
ITGLYETRIDLSQLGGD.

Residues V1134, R1217, Q1334, and R1336 above, which can be mutated from D1134, G1217, R1334, and T1336 to yield a SpVRER Cas9, are underlined and in bold.

### High Fidelity Cas9 Domains

Some aspects of the disclosure provide high fidelity Cas9 domains. In some embodiments, high fidelity Cas9 domains are engineered Cas9 domains comprising one or more mutations that decrease electrostatic interactions between the Cas9 domain and a sugar-phosphate backbone of a DNA, as compared to a corresponding wild-type Cas9 domain. Without wishing to be bound by any particular theory, high fidelity Cas9 domains that have decreased electrostatic interactions with a sugar-phosphate backbone of DNA may have less off-target effects. In some embodiments, a Cas9 domain (e.g., a wild type Cas9 domain) comprises one or more mutations that decreases the association between the Cas9 domain and a sugar-phosphate backbone of a DNA. In some embodiments, a Cas9 domain comprises one or more mutations that decreases the association between the Cas9 domain and a sugar-phosphate backbone of a DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, or at least 70%.

In some embodiments, any of the Cas9 fusion proteins provided herein comprise one or more of a N497X, a R661X, a Q695X, and/or a Q926X mutation, or a corresponding mutation in any of the amino acid sequences provided herein, wherein X is any amino acid. In some embodiments, any of the Cas9 fusion proteins provided herein comprise one or more of a N497A, a R661A, a Q695A, and/or a Q926A mutation, or a corresponding mutation in any of the amino acid sequences provided herein. In some embodiments, the Cas9 domain comprises a D10A mutation, or a corresponding mutation in any of the amino acid sequences provided herein. Cas9 domains with high fidelity are known in the art and would be apparent to the skilled artisan. For example, Cas9 domains with high fidelity have been described in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each are incorporated herein by reference.

High Fidelity Cas9 domain mutations relative to Cas9 are shown in bold and underlines

DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTAFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGALSRKLINGIRDKQSGKTILDFLKSDGFANRNFMALIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRAITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

### Nucleic Acid Programmable DNA Binding Proteins

Some aspects of the disclosure provide nucleic acid programmable DNA binding proteins, which may be used to guide a protein, such as a base editor, to a specific nucleic acid (e.g., DNA or RNA) sequence. Nucleic acid programmable DNA binding proteins include, without limitation, Cas9 (e.g., dCas9 and nCas9), CasX, CasY, Cpf1, Cas12b/C2c1, and Cas12c/C2c3. One example of a nucleic acid programmable DNA-binding protein that has different PAM specificity than Cas9 is Clustered Regularly Interspaced Short Palindromic Repeats from Prevotella and Francisella 1 (Cpf1). Similar to Cas9, Cpf1 is also a class 2 CRISPR effector, it has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

Also useful in the present compositions and methods are nuclease-inactive Cpf1 (dCpf1) variants that may be used as a guide nucleotide sequence-programmable DNA-binding protein domain. The Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity. For example, mutations corresponding to D917A, E1006A, or D1255A in Francisella novicida Cpf1 inactivate Cpf1 nuclease activity. In some embodiments, the dCpf1 of the present disclosure comprises mutations corresponding to D917A, E1006A, D1255A, D917A/E1006A, D917A/D1255A, E1006A/D1255A, or D917A/E1006A/D1255A. It is to be understood that any mutations, e.g., substitution mutations, deletions, or insertions that inactivate the RuvC domain of Cpf1, may be used in accordance with the present disclosure.

In some embodiments, the nucleic acid programmable DNA binding protein (napDNAbp) of any of the fusion proteins provided herein may be a Cpf1 protein. In some embodiments, the Cpf1 protein is a Cpf1 nickase (nCpf1). In some embodiments, the Cpf1 protein is a nuclease inactive Cpf1 (dCpf1). In some embodiments, the Cpf1, the nCpf1, or the dCpf1 comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a Cpf1 sequence disclosed herein. In some embodiments, the dCpf1 comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a Cpf1 sequence disclosed herein, and comprises mutations corresponding to D917A, E1006A, D1255A, D917A/E1006A, D917A/D1255A, E1006A/D1255A, or D917A/E1006A/D1255A. It should be appreciated that Cpf1 from other bacterial species may also be used in accordance with the present disclosure.

Wild type Francisella novicida Cpf1 (D917, E1006, and D1255 are bolded and underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFKTGGV
LRAYQLTAPFETFKKMGKQTGIIYYVPAGFTSKICPVTGFVNQLYPKYES
VSKSQEFFSKFDKICYNLDKGYFEFSFDYKNFGDKAAKGKWTIASFGSRL
INFRNSDKNHNWDTREVYPTKELEKLLKDYSIEYGHGECIKAAICGESDK
KFFAKLTSVLNTILQMRNSKTGTELDYLISPVADVNGNFFDSRQAPKNMP
QDADANGAYHIGLKGLMLLGRIKNNQEGKKLNLVIKNEEYFEFVQNRNN

Francisella novicida Cpf1 D917A (A917, E1006, and D1255 are bolded and underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN

Francisella novicida Cpf1 E1006A (D917, A1006, and D1255 are bolded and underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN

Francisella novicida Cpf1 D1255A (D917, E1006, and A1255 are bolded and underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN

Francisella novicida Cpf1 D917A/E1006A (A917, A1006, and D1255 are bolded and underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN

Francisella novicida Cpf1 D917A/D1255A (A917, E1006, and A1255 are bolded and underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN

Francisella novicida Cpf1 E1006A/D1255A (D917, A1006, and A1255 are bolded and underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN

Francisella novicida Cpf1 D917A/E1006A/D1255A (A917, A1006, and A1255 are bolded and underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN

The Cas9 nuclease has two functional endonuclease domains: RuvC and HNH. Cas9 undergoes a conformational change upon target binding that positions the nuclease domains to cleave opposite strands of the target DNA. The end result of Cas9-mediated DNA cleavage is a double-strand break (DSB) within the target DNA (˜3-4 nucleotides upstream of the PAM sequence). The resulting DSB is then repaired by one of two general repair pathways: (1) the efficient but error-prone non-homologous end joining (NHEJ) pathway; or (2) the less efficient but high-fidelity homology directed repair (HDR) pathway.

The “efficiency” of non-homologous end joining (NHEJ) and/or homology directed repair (HDR) can be calculated by any convenient method. For example, in some cases, efficiency can be expressed in terms of percentage of successful HDR. For example, a surveyor nuclease assay can be used to generate cleavage products and the ratio of products to substrate can be used to calculate the percentage. For example, a surveyor nuclease enzyme can be used that directly cleaves DNA containing a newly integrated restriction sequence as the result of successful HDR. More cleaved substrate indicates a greater percent HDR (a greater efficiency of HDR). As an illustrative example, a fraction (percentage) of HDR can be calculated using the following equation [(cleavage products)/(substrate plus cleavage products)] (e.g., (b+c)/(a+b+c), where “a” is the band intensity of DNA substrate and “b” and “c” are the cleavage products).

In some cases, efficiency can be expressed in terms of percentage of successful NHEJ. For example, a T7 endonuclease I assay can be used to generate cleavage products and the ratio of products to substrate can be used to calculate the percentage NHEJ. T7 endonuclease I cleaves mismatched heteroduplex DNA which arises from hybridization of wild-type and mutant DNA strands (NHEJ generates small random insertions or deletions (indels) at the site of the original break). More cleavage indicates a greater percent NHEJ (a greater efficiency of NHEJ). As an illustrative example, a fraction (percentage) of NHEJ can be calculated using the following equation: (1−(1−(b+c)/(a+b+c))^{1/2})×100, where “a” is the band intensity of DNA substrate and “b” and “c” are the cleavage products (Ran et. al., 2013 Sep. 12; 154(6):1380-9; and Ran et al., Nat Protoc. 2013 November; 8(11): 2281-2308).

The NHEJ repair pathway is the most active repair mechanism, and it frequently causes small nucleotide insertions or deletions (indels) at the DSB site. The randomness of NHEJ-mediated DSB repair has important practical implications, because a population of cells expressing Cas9 and a gRNA or a guide polynucleotide can result in a diverse array of mutations. In most cases, NHEJ gives rise to small indels in the target DNA that result in amino acid deletions, insertions, or frameshift mutations leading to premature stop codons within the open reading frame (ORF) of the targeted gene. The ideal end result is a loss-of-function mutation within the targeted gene.

While NHEJ-mediated DSB repair often disrupts the open reading frame of the gene, homology directed repair (HDR) can be used to generate specific nucleotide changes ranging from a single nucleotide change to large insertions like the addition of a fluorophore or tag.

In order to utilize HDR for gene editing, a DNA repair template containing the desired sequence can be delivered into the cell type of interest with the gRNA(s) and Cas9 or Cas9 nickase. The repair template can contain the desired edit as well as additional homologous sequence immediately upstream and downstream of the target (termed left & right homology arms). The length of each homology arm can be dependent on the size of the change being introduced, with larger insertions requiring longer homology arms. The repair template can be a single-stranded oligonucleotide, double-stranded oligonucleotide, or a double-stranded DNA plasmid. The efficiency of HDR is generally low (<10% of modified alleles) even in cells that express Cas9, gRNA and an exogenous repair template. The efficiency of HDR can be enhanced by synchronizing the cells, since HDR takes place during the S and G2 phases of the cell cycle. Chemically or genetically inhibiting genes involved in NHEJ can also increase HDR frequency.

In some embodiments, Cas9 is a modified Cas9. A given gRNA targeting sequence can have additional sites throughout the genome where partial homology exists. These sites are called off-targets and need to be considered when designing a gRNA. In addition to optimizing gRNA design, CRISPR specificity can also be increased through modifications to Cas9. Cas9 generates double-strand breaks (DSBs) through the combined activity of two nuclease domains, RuvC and HNH. Cas9 nickase, a D10A mutant of SpCas9, retains one nuclease domain and generates a DNA nick rather than a DSB. The nickase system can also be combined with HDR-mediated gene editing for specific gene edits.

In some cases, Cas9 is a variant Cas9 protein. A variant Cas9 polypeptide has an amino acid sequence that is different by one amino acid (e.g., has a deletion, insertion, substitution, fusion) when compared to the amino acid sequence of a wild type Cas9 protein. In some instances, the variant Cas9 polypeptide has an amino acid change (e.g., deletion, insertion, or substitution) that reduces the nuclease activity of the Cas9 polypeptide. For example, in some instances, the variant Cas9 polypeptide has less than 50%, less than 40%, less than 30%, less than 20%, less than 10%, less than 5%, or less than 1% of the nuclease activity of the corresponding wild-type Cas9 protein. In some cases, the variant Cas9 protein has no substantial nuclease activity. When a subject Cas9 protein is a variant Cas9 protein that has no substantial nuclease activity, it can be referred to as “dCas9.”

In some cases, a variant Cas9 protein has reduced nuclease activity. For example, a variant Cas9 protein exhibits less than about 20%, less than about 15%, less than about 10%, less than about 5%, less than about 1%, or less than about 0.1%, of the endonuclease activity of a wild-type Cas9 protein, e.g., a wild-type Cas9 protein.

In some cases, a variant Cas9 protein can cleave the complementary strand of a guide target sequence but has reduced ability to cleave the non-complementary strand of a double stranded guide target sequence. For example, the variant Cas9 protein can have a mutation (amino acid substitution) that reduces the function of the RuvC domain. As a non-limiting example, in some embodiments, a variant Cas9 protein has a D10A (aspartate to alanine at amino acid position 10) and can therefore cleave the complementary strand of a double stranded guide target sequence but has reduced ability to cleave the non-complementary strand of a double stranded guide target sequence (thus resulting in a single strand break (SSB) instead of a double strand break (DSB) when the variant Cas9 protein cleaves a double stranded target nucleic acid) (see, for example, Jinek et al., Science. 2012 Aug. 17; 337(6096):816-21).

In some cases, a variant Cas9 protein can cleave the non-complementary strand of a double stranded guide target sequence but has reduced ability to cleave the complementary strand of the guide target sequence. For example, the variant Cas9 protein can have a mutation (amino acid substitution) that reduces the function of the HNH domain (RuvC/HNH/RuvC domain motifs). As a non-limiting example, in some embodiments, the variant Cas9 protein has an H840A (histidine to alanine at amino acid position 840) mutation and can therefore cleave the non-complementary strand of the guide target sequence but has reduced ability to cleave the complementary strand of the guide target sequence (thus resulting in a SSB instead of a DSB when the variant Cas9 protein cleaves a double stranded guide target sequence). Such a Cas9 protein has a reduced ability to cleave a guide target sequence (e.g., a single stranded guide target sequence) but retains the ability to bind a guide target sequence (e.g., a single stranded guide target sequence).

In some cases, a variant Cas9 protein has a reduced ability to cleave both the complementary and the non-complementary strands of a double stranded target DNA. As a non-limiting example, in some cases, the variant Cas9 protein harbors both the D10A and the H840A mutations such that the polypeptide has a reduced ability to cleave both the complementary and the non-complementary strands of a double stranded target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors W476A and W1126A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors H840A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some cases, the variant Cas9 protein harbors H840A, D10A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, the variant Cas9 has restored catalytic His residue at position 840 in the Cas9 HNH domain (A840H).

As another non-limiting example, in some cases, the variant Cas9 protein harbors, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some cases, the variant Cas9 protein harbors D10A, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some cases, when a variant Cas9 protein harbors W476A and W 1126A mutations or when the variant Cas9 protein harbors P475A, W476A, N477A, D1125A, W1126A, and D1127A mutations, the variant Cas9 protein does not bind efficiently to a PAM sequence. Thus, in some such cases, when such a variant Cas9 protein is used in a method of binding, the method does not require a PAM sequence. In other words, in some cases, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a variant Cas9 protein that has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the variant Cas9 protein can still bind to target DNA in a site-specific manner (because it is still guided to a target DNA sequence by a guide RNA) as long as it retains the ability to interact with the guide RNA.

In some embodiments, the variant Cas protein can be spCas9, spCas9-VRQR, spCas9-VRER, xCas9 (sp), saCas9, saCas9-KKH, spCas9-MQKSER, spCas9-LRKIQK, or spCas9-LRVSQL.

Alternatives to S. pyogenes Cas9 can include RNA-guided endonucleases from the Cpf1 family that display cleavage activity in mammalian cells. CRISPR from Prevotella and Francisella 1 (CRISPR/Cpf1) is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. Unlike Cas9 nucleases, the result of Cpf1-mediated DNA cleavage is a double-strand break with a short 3′ overhang. Cpf1's staggered cleavage pattern can open up the possibility of directional gene transfer, analogous to traditional restriction enzyme cloning, which can increase the efficiency of gene editing. Like the Cas9 variants and orthologues described above, Cpf1 can also expand the number of sites that can be targeted by CRISPR to AT-rich regions or AT-rich genomes that lack the NGG PAM sites favored by SpCas9. The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9. Furthermore, Cpf1 does not have a HNH endonuclease domain, and the N-terminal of Cpf1 does not have the alpha-helical recognition lobe of Cas9. Cpf1 CRISPR-Cas domain architecture shows that Cpf1 is functionally unique, being classified as Class 2, type V CRISPR system. The Cpf1 loci encode Cas1, Cas2 and Cas4 proteins more similar to types I and III than from type II systems. Functional Cpf1 doesn't need the trans-activating CRISPR RNA (tracrRNA), therefore, only CRISPR (crRNA) is required. This benefits genome editing because Cpf1 is not only smaller than Cas9, but also it has a smaller sgRNA molecule (proximately half as many nucleotides as Cas9). The Cpf1-crRNA complex cleaves target DNA or RNA by identification of a protospacer adjacent motif 5′-YTN-3′ in contrast to the G-rich PAM targeted by Cas9. After identification of PAM, Cpf1 introduces a sticky-end-like DNA double-stranded break of 4 or 5 nucleotides overhang.

Fusion Proteins Comprising Two napDNAbp, a Deaminase Domain

Some aspects of the disclosure provide fusion proteins comprising a napDNAbp domain having nickase activity (e.g., nCas domain) and a catalytically inactive napDNAbp (e.g., dCas domain) and a nucleobase editor (e.g., adenosine deaminase domain, cytidine deaminase domain), where at least the napDNAbp domains are joined by a linker. It should be appreciated that the Cas domains may be any of the Cas domains or Cas proteins (e.g., dCas9 and nCas9) provided herein. In some embodiments, any of the Cas domains, DNA binding protein domains, or Cas proteins include, without limitation, Cas9 (e.g., dCas9 and nCas9), Cas12a/Cpf1, Cas12b/C2c1, Cas12c/C2c3, Cas12d/CasY, Cas12e/CasX, Cas12g, Cas12h, and Cas12i. One example of a programmable polynucleotide-binding protein that has different PAM specificity than Cas9 is Clustered Regularly Interspaced Short Palindromic Repeats from Prevotella and Francisella1 (Cpf1). Similar to Cas9, Cpf1 is also a class 2 CRISPR effector. For example, and without limitation, in some embodiments, the fusion protein comprises the structure, where the deaminase is adenosine deaminase or cytidine deaminase:

NH₂-[deaminase]-[nCas domain]-[dCas domain]-COOH;
NH₂-[deaminase]-[dCas domain]-[nCas domain]-COOH;
NH₂-[nCas domain]-[dCas domain]-[deaminase]-COOH;
NH₂-[dCas domain]-[nCas domain]-[deaminase]-COOH;
NH₂-[nCas domain]-[deaminase]-[dCas domain]-COOH;
NH₂-[dCas domain]-[deaminase]-[nCas domain]-COOH;

In some embodiments, the “-” used in the general architecture above indicates the presence of an optional linker. In some embodiments, the deaminase and a napDNAbp (e.g., Cas domain) are not joined by a linker sequence, but are directly fused. In some embodiments, a linker is present between the deaminase domain and the napDNAbp. In some embodiments, the deaminase or other nucleobase editor is directly fused to dCas and a linker joins dCas and nCas9. In some embodiments, the deaminase and the napDNAbps are fused via any of the linkers provided herein. For example, in some embodiments the deaminase and the napDNAbp are fused via any of the linkers provided below in the section entitled “Linkers”. In some embodiments, the dCas domain and the deaminase are immediately adjacent and the nCas domain is joined to these domains (either 5′ or 3′) via a linker.

### Protospacer Adjacent Motif

The term “protospacer adjacent motif (PAM)” or PAM-like motif refers to a 2-6 base pair DNA sequence immediately following the DNA sequence targeted by the Cas9 nuclease in the CRISPR bacterial adaptive immune system. In some embodiments, the PAM can be a 5′ PAM (i.e., located upstream of the 5′ end of the protospacer). In other embodiments, the PAM can be a 3′ PAM (i.e., located downstream of the 5′ end of the protospacer).

The PAM sequence is essential for target binding, but the exact sequence depends on a type of Cas protein.

A base editor provided herein can comprise a CRISPR protein-derived domain that is capable of binding a nucleotide sequence that contains a canonical or non-canonical protospacer adjacent motif (PAM) sequence. A PAM site is a nucleotide sequence in proximity to a target polynucleotide sequence. Some aspects of the disclosure provide for base editors comprising all or a portion of CRISPR proteins that have different PAM specificities. For example, typically Cas9 proteins, such as Cas9 from S. pyogenes (spCas9), require a canonical NGG PAM sequence to bind a particular nucleic acid region, where the “N” in “NGG” is adenine (A), thymine (T), guanine (G), or cytosine (C), and the G is guanine. A PAM can be CRISPR protein-specific and can be different between different base editors comprising different CRISPR protein-derived domains. A PAM can be 5′ or 3′ of a target sequence. A PAM can be upstream or downstream of a target sequence. A PAM can be 1, 2, 3, 4, 5, 6, 7, 8, 9, 10 or more nucleotides in length. Often, a PAM is between 2-6 nucleotides in length. Several PAM variants are described in Table 1.

In some embodiments, the SpCas9 has specificity for PAM nucleic acid sequence 5′-NGC-3′ or 5′-NGG-3′. In various embodiments of the above aspects, the SpCas9 is a Cas9 or Cas9 variant listed in Table 1. In various embodiments of the above aspects, the modified SpCas9 is spCas9-MQKFRAER. In some embodiments, the variant Cas protein can be spCas9, spCas9-VRQR, spCas9-VRER, xCas9 (sp), saCas9, saCas9-KKH, SpCas9-MQKFRAER, spCas9-MQKSER, spCas9-LRKIQK, or spCas9-LRVSQL. In one specific embodiment, a modified SpCas9 including amino acid substitutions D1135M, S1136Q, G1218K, E1219F, A1322R, D1332A, R1335E, and T1337R (SpCas9-MQKFRAER) and having specificity for the altered PAM 5′-NGC-3′ is used.

In some embodiments, the PAM is NGT. In some embodiments, the NGT PAM is a variant. In some embodiments, the NGT PAM variant is created through targeted mutations at one or more residues 1335, 1337, 1135, 1136, 1218, and/or 1219. In some embodiments, the NGT PAM variant is created through targeted mutations at one or more residues 1219, 1335, 1337, 1218. In some embodiments, the NGT PAM variant is created through targeted mutations at one or more residues 1135, 1136, 1218, 1219, and 1335. In some embodiments, the NGT PAM variant is selected from the set of targeted mutations provided in Tables 4 and 5 below.

TABLE 4
NGT PAM Variant Mutations at
residues 1219, 1335, 1337, 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |
| 9 | L | L | T | |
| 10 | L | L | R | |
| 11 | L | L | Q | |
| 12 | L | L | L | |
| 13 | F | I | T | |
| 14 | F | I | R | |
| 15 | F | I | Q | |
| 16 | F | I | L | |
| 17 | F | G | C | |
| 18 | H | L | N | |
| 19 | F | G | C | A |
| 20 | H | L | N | V |
| 21 | L | A | W | |
| 22 | L | A | F | |
| 23 | L | A | Y | |
| 24 | I | A | W | |
| 25 | I | A | F | |
| 26 | I | A | Y | |

TABLE 5
NGT PAM Variant Mutations at residues
1135, 1136, 1218, 1219, and 1335

| Variant | D1135L | S1136R | G1218S | E1219V | R1335Q |
|---|---|---|---|---|---|
| 27 | G | | | | |
| 28 | V | | | | |
| 29 | I | | | | |
| 30 | | A | | | |
| 31 | | W | | | |
| 32 | | H | | | |
| 33 | | K | | | |
| 34 | | | K | | |
| 35 | | | R | | |
| 36 | | | Q | | |
| 37 | | | T | | |
| 38 | | | N | | |
| 39 | | | | I | |
| 40 | | | | A | |
| 41 | | | | N | |
| 42 | | | | Q | |
| 43 | | | | G | |
| 44 | | | | L | |
| 45 | | | | S | |
| 46 | | | | T | |
| 47 | | | | | L |
| 48 | | | | | I |
| 49 | | | | | V |
| 50 | | | | | N |
| 51 | | | | | S |
| 52 | | | | | T |
| 53 | | | | | F |
| 54 | | | | | Y |
| 55 | N1286Q | I1331F | | | |

In some embodiments, the NGT PAM variant is selected from variant 5, 7, 28, 31, or 36 in Tables 2 and 3. In some embodiments, the variants have improved NGT PAM recognition.

In some embodiments, the NGT PAM variants have mutations at residues 1219, 1335, 1337, and/or 1218. In some embodiments, the NGT PAM variant is selected with mutations for improved recognition from the variants provided in Table 6 below.

TABLE 6
NGT PAM Variant Mutations at residues 1219, 1335, 1337, and 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |

In some embodiments, the NGT PAM is selected from the variants provided in Table 7 below.

TABLE 7
NGT PAM variants

| | NGTN | | | | | | | |
|---|---|---|---|---|---|---|---|---|
| | variant | D1135 | S1136 | G1218 | E1219 | A1322R | R1335 | T1337 |
| Variant 1 | LRKIQK | L | R | K | I | — | Q | K |
| Variant 2 | LRSVQK | L | R | S | V | — | Q | K |
| Variant 3 | LRSVQL | L | R | S | V | — | Q | L |
| Variant 4 | LRKIRQK | L | R | K | I | R | Q | K |
| Variant 5 | LRSVRQK | L | R | S | V | R | Q | K |
| Variant 6 | LRSVRQL | L | R | S | V | R | Q | L |

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises a D9X mutation, or a corresponding mutation in any of the amino acid sequences provided herein may be fused with any of the cytidine deaminases or adenosine deaminases provided herein

In some embodiments, the SpCas9 domain comprises one or more of a D1135X, a R1335X, and a T1336X mutation, or a corresponding mutation in any of the amino acid sequences provided herein, wherein X is any amino acid. In some embodiments, the SpCas9 domain comprises one or more of a D1135E, R1335Q, and T1336R mutation, or a corresponding mutation in any of the amino acid sequences provided herein. In some embodiments, the SpCas9 domain comprises a D1135E, a R1335Q, and a T1336R mutation, or corresponding mutations in any of the amino acid sequences provided herein. In some embodiments, the SpCas9 domain comprises one or more of a D1135X, a R1335X, and a T1336X mutation, or a corresponding mutation in any of the amino acid sequences provided herein, wherein X is any amino acid. In some embodiments, the SpCas9 domain comprises one or more of a D1135V, a R1335Q, and a T1336R mutation, or a corresponding mutation in any of the amino acid sequences provided herein. In some embodiments, the SpCas9 domain comprises a D1135V, a R1335Q, and a T1336R mutation, or corresponding mutations in any of the amino acid sequences provided herein. In some embodiments, the SpCas9 domain comprises one or more of a D1135X, a G1217X, a R1335X, and a T1336X mutation, or a corresponding mutation in any of the amino acid sequences provided herein, wherein X is any amino acid. In some embodiments, the SpCas9 domain comprises one or more of a D1135V, a G1217R, a R1335Q, and a T1336R mutation, or a corresponding mutation in any of the amino acid sequences provided herein. In some embodiments, the SpCas9 domain comprises a D1135V, a G1217R, a R1335Q, and a T1336R mutation, or corresponding mutations in any of the amino acid sequences provided herein.

In some embodiments, the Cas9 domains of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to a Cas9 polypeptide described herein. In some embodiments, the Cas9 domains of any of the fusion proteins provided herein comprises the amino acid sequence of any Cas9 polypeptide described herein. In some embodiments, the Cas9 domains of any of the fusion proteins provided herein consists of the amino acid sequence of any Cas9 polypeptide described herein.

In some examples, a PAM recognized by a CRISPR protein-derived domain of a base editor disclosed herein can be provided to a cell on a separate oligonucleotide to an insert (e.g., an AAV insert) encoding the base editor. In such embodiments, providing PAM on a separate oligonucleotide can allow cleavage of a target sequence that otherwise would not be able to be cleaved, because no adjacent PAM is present on the same polynucleotide as the target sequence.

In an embodiment, S. pyogenes Cas9 (SpCas9) can be used as a CRISPR endonuclease for genome engineering. However, others can be used. In some embodiments, a different endonuclease can be used to target certain genomic targets. In some embodiments, synthetic SpCas9-derived variants with non-NGG PAM sequences can be used. Additionally, other Cas9 orthologues from various species have been identified and these “non-SpCas9s” can bind a variety of PAM sequences that can also be useful for the present disclosure. For example, the relatively large size of SpCas9 (approximately 4 kilobase (kb) coding sequence) can lead to plasmids carrying the SpCas9 cDNA that cannot be efficiently expressed in a cell. Conversely, the coding sequence for Staphylococcus aureus Cas9 (SaCas9) is approximately 1 kilobase shorter than SpCas9, possibly allowing it to be efficiently expressed in a cell. Similar to SpCas9, the SaCas9 endonuclease is capable of modifying target genes in mammalian cells in vitro and in mice in vivo. In some embodiments, a Cas protein can target a different PAM sequence. In some embodiments, a target gene can be adjacent to a Cas9 PAM, 5′-NGG, for example. In other embodiments, other Cas9 orthologs can have different PAM requirements. For example, other PAMs such as those of S. thermophilus (5′-NNAGAA for CRISPR1 and 5′-NGGNG for CRISPR3) and Neisseria meningiditis (5′-NNNNGATT) can also be found adjacent to a target gene.

In some embodiments, for a S. pyogenes system, a target gene sequence can precede (i.e., be 5′ to) a 5′-NGG PAM, and a 20-nt guide RNA sequence can base pair with an opposite strand to mediate a Cas9 cleavage adjacent to a PAM. In some embodiments, an adjacent cut can be or can be about 3 base pairs upstream of a PAM. In some embodiments, an adjacent cut can be or can be about 10 base pairs upstream of a PAM. In some embodiments, an adjacent cut can be or can be about 0-20 base pairs upstream of a PAM. For example, an adjacent cut can be next to, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs upstream of a PAM. An adjacent cut can also be downstream of a PAM by 1 to 30 base pairs. The sequences of exemplary SpCas9 proteins capable of binding a PAM sequence follow:

The amino acid sequence of an exemplary PAM-binding SpCas9 is as follows:

MDKKYSIGLDIGTNSVGWAVITDEYKVPSKKFKVLGNTDRHSIKKNLI
GALLFDSGETAEATRLKRTARRRYTRRKNRICYLQEIFSNEMAKVDDS
FFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTY
NQLFEENPINASGVDAKAILSARLSKSRRLENLIAQLPGEKKNGLFGN
LIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLK
ALVRQQLPEKYKEIFFDQSKNGYAGYIDGGASQEEFYKFIKPILEKMD
GTEELLVKLNREDLLRKQRTFDNGSIPHQIHLGELHAILRRQEDFYPF
LKDNREKIEKILTFRIPYYVGPLARGNSRFAWMTRKSEETITPWNFEE
VVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLT
LFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSL
HEHIANLAGSPAIKKGILQTVKVVDELVKVMGRHKPENIVIEMARENQ
TTQKGQKNSRERMKRIEEGIKELGSQILKEHPVENTQLQNEKLYLYYL
QNGRDMYVDQELDINRLSDYDVDHIVPQSFLKDDSIDNKVLTRSDKNR
GKSDNVPSEEVVKKMKNYWRQLLNAKLITQRKFDNLTKAERGGLSELD
KAGFIKRQLVETRQITKHVAQILDSRMNTKYDENDKLIREVKVITLKS
KLVSDFRKDFQFYKVREINNYHHAHDAYLNAVVGTALIKKYPKLESEF
VYGDYKVYDVRKMIAKSEQEIGKATAKYFFYSNIMNFFKTEITLANGE
IRKRPLIETNGETGEIVWDKGRDFATVRKVLSMPQVNIVKKTEVQTGG
FSKESILPKRNSDKLIARKKDWDPKKYGGFDSPTVAYSVLVVAKVEKG
KSKKLKSVKELLGITIMERSSFEKNPIDFLEAKGYKEVKKDLIIKLPK
YSLFELENGRKRMLASAGELQKGNELALPSKYVNFLYLASHYEKLKGS
PEDNEQKQLFVEQHKHYLDEIIEQISEFSKRVILADANLDKVLSAYNK
HRDKPIREQAENIIHLFTLTNLGAPAAFKYFDTTIDRKRYTSTKEVLD
ATLIHQSITGLYETRIDLSQLGGD.

The amino acid sequence of an exemplary PAM-binding SpCas9n is as follows:

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

The amino acid sequence of an exemplary PAM-binding SpEQR Cas9 is as follows:

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFESPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKQYRSTKEVLDATLIHQS
ITGLYETRIDLSQLGGD.

In this sequence, residues E1135, Q1335 and R1337, which can be mutated from D1135, R1335, and T1337 to yield a SpEQR Cas9, are underlined and in bold.

The amino acid sequence of an exemplary PAM-binding SpVQR Cas9 is as follows:

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKQYRSTKEVLDATLIHQ
SITGLYETRIDLSQLGGD.

In this sequence, residues V1135, Q1335, and R1336, which can be mutated from D1135, R1335, and T1336 to yield a SpVQR Cas9, are underlined and in bold.

The amino acid sequence of an exemplary PAM-binding SpVRER Cas9 is as follows:

MDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKD
FQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIAR
KKDWDPKKYGGFVSPTVAYSVLVVAKVEKGKSKKLKSVKELLGITIMERSS
FEKNPIDFLEAKGYKEVKKDLIIKLPKYSLFELENGRKRMLASARELQKGN
ELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKY
FDTTIDRKEYRSTKEVLDATLIHQSITGLYETRIDLSQLGGD.

In some embodiments, the Cas9 domain is a recombinant Cas9 domain. In some embodiments, the recombinant Cas9 domain is a SpyMacCas9 domain. In some embodiments, the SpyMacCas9 domain is a nuclease active SpyMacCas9, a nuclease inactive SpyMacCas9 (SpyMacCas9d), or a SpyMacCas9 nickase (SpyMacCas9n). In some embodiments, the SaCas9 domain, the SaCas9d domain, or the SaCas9n domain can bind to a nucleic acid sequence having a non-canonical PAM. In some embodiments, the SpyMacCas9 domain, the SpCas9d domain, or the SpCas9n domain can bind to a nucleic acid sequence having a NAA PAM sequence.

### Exemplary SpyMacCas9

MDKKYSIGLDIGTNSVGWAVITDDYKVPSKKFKVLGNTDRHSIKKNLIGA
LLFGSGETAEATRLKRTARRRYTRRKNRICYLQEIFSNEMAKVDDSFFHR
LEESFLVEEDKKHERHPIFGNIVDEVAYHEKYPTIYHLRKKLADSTDKAD
LRLIYLALAHMIKFRGHFLIEGDLNPDNSDVDKLFIQLVQIYNQLFEENP
INASRVDAKAILSARLSKSRRLENLIAQLPGEKRNGLFGNLIALSLGLTP
NFKSNFDLAEDAKLQLSKDTYDDDLDNLLAQIGDQYADLFLAAKNLSDAI
LLSDILRVNSEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGAYHDLLKI
IKDKDFLDNEENEDILEDIVLTLTLFEDRGMIEERLKTYAHLFDDKVMKQ
LKRRRYTGWGRLSRKLINGIRDKQSGKTILDFLKSDGFANRNFMQLIHDD
SLTFKEDIQKAQVSGQGHSLHEQIANLAGSPAIKKGILQTVKIVDELVKV
MGHKPENIVIEMARENQTTQKGQKNSRERMKRIEEGIKELGSQILKEHPV
ENTQLQNEKLYLYYLQNGRDMYVDQELDINRLSDYDVDHIVPQSFIKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEIQ
TVGQNGGLFDDNPKSPLEVTPSKLVPLKKELNPKKYGGYQKPTTAYPVLL
ITDTKQLIPISVMNKKQFEQNPVKFLRDRGYQQVGKNDFIKLPKYTLVDI
GDGIKRLWASSKEIHKGNQLVVSKKSQILLYHAHHLDSDLSNDYLQNHNQ
QFDVLFNEIISFSKKCKLGKEHIQKIENVYSNKKNSASIEELAESFIKLL
GFTQLGATSPFNFLGVKLNQKQYKGKKDYILPCTEGTLIRQSITGLYETR
VDLSKIGED.

In some cases, a variant Cas9 protein harbors, H840A, P475A, W476A, N477A, D1125A, W1126A, and D1218A mutations such that the polypeptide has a reduced ability to cleave a target DNA or RNA. Such a Cas9 protein has a reduced ability to cleave a target DNA (e.g., a single stranded target DNA) but retains the ability to bind a target DNA (e.g., a single stranded target DNA). As another non-limiting example, in some cases, the variant Cas9 protein harbors D10A, H840A, P475A, W476A, N477A, D1125A, W1126A, and D1218A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some cases, when a variant Cas9 protein harbors W476A and W 1126A mutations or when the variant Cas9 protein harbors P475A, W476A, N477A, D1125A, W1126A, and D1218A mutations, the variant Cas9 protein does not bind efficiently to a PAM sequence. Thus, in some such cases, when such a variant Cas9 protein is used in a method of binding, the method does not require a PAM sequence. In other words, in some cases, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable. In some embodiments, a CRISPR protein-derived domain of a base editor can comprise all or a portion of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the Cas9 domain may be replaced with a guide nucleotide sequence-programmable DNA-binding protein domain that has no requirements for a PAM sequence.

In some embodiments, the nucleic acid programmable DNA binding protein (napDNAbp) is a single effector of a microbial CRISPR-Cas system. Single effectors of microbial CRISPR-Cas systems include, without limitation, Cas9, Cpf1, Cas12b/C2c1, and Cas12c/C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (Cas12b/C2c1 and Cas12c/C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, Cas12b/C2c1 and Cas12c/C2c3, contain RuvC-like endonuclease domains related to Cpf1. A third system, contains an effector with two predicated HEPN RNase domains. Production of mature CRISPR RNA is tracrRNA-independent, unlike production of CRISPR RNA by Cas12b/C2c1. Cas12b/C2c1 depends on both CRISPR RNA and tracrRNA for DNA cleavage.

The crystal structure of Alicyclobaccillus acidoterrastris Cas12b/C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris Cas12b/C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with Cas12b/C2c1-mediated cleavage resulting in a staggered seven-nucleotide break of target DNA. Structural comparisons between Cas12b/C2c1 ternary complexes and previously identified Cas9 and Cpf1 counterparts demonstrate the diversity of mechanisms used by CRISPR-Cas9 systems.

In some embodiments, the nucleic acid programmable DNA binding protein (napDNAbp) of any of the fusion proteins provided herein may be a Cas12b/C2c1, or a Cas12c/C2c3 protein. In some embodiments, the napDNAbp is a Cas12b/C2c1 protein. In some embodiments, the napDNAbp is a Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12b/C2c1 or Cas12c/C2c3 protein. In some embodiments, the napDNAbp is a naturally-occurring Cas12b/C2c1 or Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of the napDNAbp sequences provided herein. It should be appreciated that Cas12b/C2c1 or Cas12c/C2c3 from other bacterial species may also be used in accordance with the present disclosure. CRISPR-Cas12b is described, for example, by Teng et al., Cell Discovery (2018) 4:63, which is incorporated therein by reference in its entirety.

Cas12b/C2c1 (uniprot.org/uniprot/TOD7A2#2)

spTOD7A2|C2C1_ALIAG CRISPR-associated endo-nuclease C2c1 OS=Alicyclobacillus acido-terrestris (strain ATCC 49025/DSM 3922/CIP 106132/NCIMB 13137/GD3B) GN=c2c1 PE=1 SV=1

MAVKSIKVKLRLDDMPEIRAGLWKLHKEVNAGVRYYTEWLSLLRQENLYR
RSPNGDGEQECDKTAEECKAELLERLRARQVENGHRGPAGSDDELLQLAR
QLYELLVPQAIGAKGDAQQIARKFLSPLADKDAVGGLGIAKAGNKPRWVR
MREAGEPGWEEEKEKAETRKSADRTADVLRALADFGLKPLMRVYTDSEMS
SVEWKPLRKGQAVRTWDRDMFQQAIERMMSWESWNQRVGQEYAKLVEQKN
RFEQKNFVGQEHLVHLVNQLQQDMKEASPGLESKEQTAHYVTGRALRGSD
KVFEKWGKLAPDAPFDLYDAEIKNVQRRNTRRFGSHDLFAKLAEPEYQAL
WREDASFLTRYAVYNSILRKLNHAKMFATFTLPDATAHPIWTRFDKLGGN
LHQYTFLFNEFGERRHAIRFHKLLKVENGVAREVDDVTVPISMSEQLDNL
LPRDPNEPIALYFRDYGAEQHFTGEFGGAKIQCRRDQLAHMHRRRGARDV
YLNVSVRVQSQSEARGERRPPYAAVFRLVGDNHRAFVHFDKLSDYLAEHP
DDGKLGSEGLLSGLRVMSVDLGLRTSASISVFRVARKDELKPNSKGRVPF
FFPIKGNDNLVAVHERSQLLKLPGETESKDLRAIREERQRTLRQLRTQLA
YLRLLVRCGSEDVGRRERSWAKLIEQPVDAANHMTPDWREAFENELQKLK
SLHGICSDKEWMDAVYESVRRVWRHMGKQVRDWRKDVRSGERPKIRGYAK
DVVGGNSIEQIEYLERQYKFLKSWSFFGKVSGQVIRAEKGSRFAITLREH
IDHAKEDRLKKLADRIIMEALGYVYALDERGKGKWVAKYPPCQLILLEEL
SEYQFNNDRPPSENNQLMQWSHRGVFQELINQAQVHDLLVGTMYAAFSSR
FDARTGAPGIRCRRVPARCTQEHNPEPFPWWLNKFVVEHTLDACPLRADD
LIPTGEGEIFVSPFSAEEGDFHQIHADLNAAQNLQQRLWSDFDISQIRLR
CDWGEVDGELVLIPRLTGKRTADSYSNKVFYTNTGVTYYERERGKKRRKV
FAQEKLSEEEAELLVEADEAREKSVVLMRDPSGIINRGNWTRQKEFWSMV
NQRIEGYLVKQIRSRVPLQDSACENTGDI

AacCas12b (Alicyclobacillus acidiphilus)—WP_067623834

MAVKSMKVKLRLDNMPEIRAGLWKLHTEVNAGVRYYTEWLSLLRQENLYR
RSPNGDGEQECYKTAEECKAELLERLRARQVENGHCGPAGSDDELLQLAR
QLYELLVPQAIGAKGDAQQIARKFLSPLADKDAVGGLGIAKAGNKPRWVR
MREAGEPGWEEEKAKAEARKSTDRTADVLRALADFGLKPLMRVYTDSDMS
SVQWKPLRKGQAVRTWDRDMFQQAIERMMSWESWNQRVGEAYAKLVEQKS
RFEQKNFVGQEHLVQLVNQLQQDMKEASHGLESKEQTAHYLTGRALRGSD
KVFEKWEKLDPDAPFDLYDTEIKNVQRRNTRRFGSHDLFAKLAEPKYQAL
WREDASFLTRYAVYNSIVRKLNHAKMFATFTLPDATAHPIWTRFDKLGGN
LHQYTFLFNEFGEGRHAIRFQKLLTVEDGVAKEVDDVTVPISMSAQLDDL
LPRDPHELVALYFQDYGAEQHLAGEFGGAKIQYRRDQLNHLHARRGARDV
YLNLSVRVQSQSEARGERRPPYAAVFRLVGDNHRAFVHFDKLSDYLAEHP
DDGKLGSEGLLSGLRVMSVDLGLRTSASISVFRVARKDELKPNSEGRVPF
CFPIEGNENLVAVHERSQLLKLPGETESKDLRAIREERQRTLRQLRTQLA
YLRLLVRCGSEDVGRRERSWAKLIEQPMDANQMTPDWREAFEDELQKLKS
LYGICGDREWTEAVYESVRRVWRHMGKQVRDWRKDVRSGERPKIRGYQKD
VVGGNSIEQIEYLERQYKFLKSWSFFGKVSGQVIRAEKGSRFAITLREHI
DHAKEDRLKKLADRIIMEALGYVYALDDERGKGKWVAKYPPCQLILLEEL
SEYQFNNDRPPSENNQLMQWSHRGVFQELLNQAQVHDLLVGTMYAAFSSR
FDARTGAPGIRCRRVPARCAREQNPEPFPWWLNKFVAEHKLDGCPLRADD
LIPTGEGEFFVSPFSAEEGDFHQIHADLNAAQNLQRRLWSDFDISQIRLR
CDWGEVDGEPVLIPRTTGKRTADSYGNKVFYTKTGVTYYERERGKKRRKV
FAQEELSEEEAELLVEADEAREKSVVLMRDPSGIINRGDWTRQKEFWSMV
NQRIEGYLVKQIRSRVRLQESACENTGDI

BvCas12b (Bacillus sp. V3-13) NCBI Reference Sequence: WP_101661451.1

MAIRSIKLKMKTNSGTDSIYLRKALWRTHQLINEGIAYYMNLLTLYRQEA
IGDKTKEAYQAELINIIRNQQRNNGSSEEHGSDQEILALLRQLYELIIPS
SIGESGDANQLGNKFLYPLVDPNSQSGKGTSNAGRKPRWKRLKEEGNPDW
ELEKKKDEERKAKDPTVKIFDNLNKYGLLPLFPLFTNIQKDIEWLPLGKR
QSVRKWDKDMFIQAIERLLSWESWNRRVADEYKQLKEKTESYYKEHLTGG
EEWIEKIRKFEKERNMELEKNAFAPNDGYFITSRQIRGWDRVYEKWSKLP
ESASPEELWKVVAEQQNKMSEGFGDPKVFSFLANRENRDIWRGHSERIYH
IAAYNGLQKKLSRTKEQATFTLPDAIEHPLWIRYESPGGTNLNLFKLEEK
QKKNYYVTLSKIIWPSEEKWIEKENIEIPLAPSIQFNRQIKLKQHVKGKQ
EISFSDYSSRISLDGVLGGSRIQFNRKYIKNHKELLGEGDIGPVFFNLVV
DVAPLQETRNGRLQSPIGKALKVISSDFSKVIDYKPKELMDWMNTGSASN
SFGVASLLEGMRVMSIDMGQRTSASVSIFEVVKELPKDQEQKLFYSINDT
ELFAIHKRSFLLNLPGEVVTKNNKQQRQERRKKRQFVRSQIRMLANVLRL
ETKKTPDERKKAIHKLMEIVQSYDSWTASQKEVWEKELNLLTNMAAFNDE
IWKESLVELHHRIEPYVGQIVSKWRKGLSEGRKNLAGISMWNIDELEDTR
RLLISWSKRSRTPGEANRIETDEPFGSSLLQHIQNVKDDRLKQMANLIIM
TALGFKYDKEEKDRYKRWKETYPACQIILFENLNRYLFNLDRSRRENSRL
MKWAHRSIPRTVSMQGEMFGLQVGDVRSEYSSRFHAKTGAPGIRCHALTE
EDLKAGSNTLKRLIEDGFINESELAYLKKGDIIPSQGGELFVTLSKRYKK
DSDNNELTVIHADINAAQNLQKRFWQQNSEVYRVPCQLARMGEDKLYIPK
SQTETIKKYFGKGSFVKNNTEQEVYKWEKSEKMKIKTDTTFDLQDLDGFE
DISKTIELAQEQQKKYLTMFRDPSGYFFNNETWRPQKEYWSIVNNIIKSC
LKKKILSNKVEL

BhCas12b (Bacillus hisashii) NCBI Reference Sequence: WP_095142515

MAPKKKRKVGIHGVPAAATRSFILKIEPNEEVKKGLWKTHEVLNHGIAYY
MNILKLIRQEAIYEHHEQDPKNPKKVSKAEIQAELWDFVLKMQKCNSFTH
EVDKDEVFNILRELYEELVPSSVEKKGEANQLSNKFLYPLVDPNSQSGKG
TASSGRKPRWYNLKIAGDPSWEEEKKKWEEDKKKDPLAKILGKLAEYGLI
PLFIPYTDSNEPIVKEIKWMEKSRNQSVRRLDKDMFIQALERFLSWESWN
LKVKEEYEKVEKEYKTLEERIKEDIQALKALEQYEKERQEQLLRDTLNTN
EYRLSKRGLRGWREIIQKWLKMDENEPSEKYLEVFKDYQRKHPREAGDYS
VYEFLSKKENHFIWRNHPEYPYLYATFCEIDKKKKDAKQQATFTLADPIN
HPLWVRFEERSGSNLNKYRILTEQLHTEKLKKKLTVQLDRLIYPTESGGW
EEKGKVDIVLLPSRQFYNQIFLDIEEKGKHAFTYKDESIKFPLKGTLGGA
RVQFDRDHLRRYPHKVESGNVGRIYFNMTVNIEPTESPVSKSLKIHRDDF
PKVVNFKPKELTEWIKDSKGKKLKSGIESLEIGLRVMSIDLGQRQAAAAS
IFEVVDQKPDIEGKLFFPIKGTELYAVHRASFNIKLPGETLVKSREVLRK
AREDNLKLMNQKLNFLRNVLHFQQFEDITEREKRVTKWISRQENSDVPLV
YQDELIQIRELMYKPYKDWVAFLKQLHKRLEVEIGKEVKHWRKSLSDGRK
GLYGISLKNIDEIDRTRKFLLRWSLRPTEPGEVRRLEPGQRFAIDQLNHL
NALKEDRLKKMANTIIMHALGYCYDVRKKKWQAKNPACQIILFEDLSNYN
PYEERSRFENSKLMKWSRREIPRQVALQGEIYGLQVGEVGAQFSSRFHAK
TGSPGIRCSVVTKEKLQDNRFFKNLQREGRLTLDKIAVLKEGDLYPDKGG
EKFISLSKDRKCVTTHADINAAQNLQKRFWTRTHGFYKVYCKAYQVDGQT
VYIPESKDQKQKIIEEFGEGYFILKDGVYEWVNAGKLKIKKGSSKQSSSE
LVDSDILKDSFDLASELKGEKLMLYRDPSGNVFPSDKWMAAGVFFGKLER
ILISKLTNQYSISTIEDDSSKQSMKRPAATKKAGQAKKKK

including the variant termed BvCas12b V4 (S893R/K846R/E837G changes rel. to wt above)

BhCas12b (V4) is expressed as follows: 5′ mRNA Cap---5′UTR---bhCas12b---STOP sequence---3′UTR---120polyA tail

5′UTR:
GGGAAATAAGAGAGAAAAGAAGAGTAAGAAGAAATATAAGAGCCACC
3′ UTR (TriLink standard UTR)
GCTGGAGCCTCGGTGGCCATGCTTCTTGCCCCTTGGGCCTCCCCCCAGC
CCCTCCTCCCCTTCCTGCACCCGTACCCCCGTGGTCTTTGAATAAAGTC
TGA

Nucleic acid sequence of bhCas12b (V4)

ATGGCCCCAAAGAAGAAGCGGAAGGTCGGTATCCACGGAGTCCCAGCAGC
CGCCACCAGATCCTTCATCCTGAAGATCGAGCCCAACGAGGAAGTGAAGA
AAGGCCTCTGGAAAACCCACGAGGTGCTGAACCACGGAATCGCCTACTAC
ATGAATATCCTGAAGCTGATCCGGCAAGAGGCCATCTACGAGCACCACGA
GCAGGACCCCAAGAATCCCAAGAAGGTGTCCAAGGCCGAGATCCAGGCCG
AGCTGTGGGATTTCGTGCTGAAGATGCAGAAGTGCAACAGCTTCACACAC
GAGGTGGACAAGGACGAGGTGTTCAACATCCTGAGAGAGCTGTACGAGGA
ACTGGTGCCCAGCAGCGTGGAAAAGAAGGGCGAAGCCAACCAGCTGAGCA
ACAAGTTTCTGTACCCTCTGGTGGACCCCAACAGCCAGTCTGGAAAGGGA
ACAGCCAGCAGCGGCAGAAAGCCCAGATGGTACAACCTGAAGATTGCCGG
CGATCCCTCCTGGGAAGAAGAGAAGAAGAAGTGGGAAGAAGATAAGAAAA
AGGACCCGCTGGCCAAGATCCTGGGCAAGCTGGCTGAGTACGGACTGATC
CCTCTGTTCATCCCCTACACCGACAGCAACGAGCCCATCGTGAAAGAAAT
CAAGTGGATGGAAAAGTCCCGGAACCAGAGCGTGCGGCGGCTGGATAAGG
ACATGTTCATTCAGGCCCTGGAACGGTTCCTGAGCTGGGAGAGCTGGAAC
CTGAAAGTGAAAGAGGAATACGAGAAGGTCGAGAAAGAGTACAAGACCCT
GGAAGAGAGGATCAAAGAGGACATCCAGGCTCTGAAGGCTCTGGAACAGT
ATGAGAAAGAGCGGCAAGAACAGCTGCTGCGGGACACCCTGAACACCAAC
GAGTACCGGCTGAGCAAGAGAGGCCTTAGAGGCTGGCGGGAAATCATCCA
GAAATGGCTGAAAATGGACGAGAACGAGCCCTCCGAGAAGTACCTGGAAG
TGTTCAAGGACTACCAGCGGAAGCACCCTAGAGAGGCCGGCGATTACAGC
GTGTACGAGTTCCTGTCCAAGAAAGAGAACCACTTCATCTGGCGGAATCA
CCCTGAGTACCCCTACCTGTACGCCACCTTCTGCGAGATCGACAAGAAAA
AGAAGGACGCCAAGCAGCAGGCCACCTTCACACTGGCCGATCCTATCAAT
CACCCTCTGTGGGTCCGATTCGAGGAAAGAAGCGGCAGCAACCTGAACAA
GTACAGAATCCTGACCGAGCAGCTGCACACCGAGAAGCTGAAGAAAAAGC
TGACAGTGCAGCTGGACCGGCTGATCTACCCTACAGAATCTGGCGGCTGG
GAAGAGAAGGGCAAAGTGGACATTGTGCTGCTGCCCAGCCGGCAGTTCTA
CAACCAGATCTTCCTGGACATCGAGGAAAAGGGCAAGCACGCCTTCACCT
ACAAGGATGAGAGCATCAAGTTCCCTCTGAAGGGCACACTCGGCGGAGCC
AGAGTGCAGTTCGACAGAGATCACCTGAGAAGATACCCTCACAAGGTGGA
AAGCGGCAACGTGGGCAGAATCTACTTCAACATGACCGTGAACATCGAGC
CTACAGAGTCCCCAGTGTCCAAGTCTCTGAAGATCCACCGGGACGACTTC
CCCAAGGTGGTCAACTTCAAGCCCAAAGAACTGACCGAGTGGATCAAGGA
CAGCAAGGGCAAGAAACTGAAGTCCGGCATCGAGTCCCTGGAAATCGGCC
TGAGAGTGATGAGCATCGACCTGGGACAGAGACAGGCCGCTGCCGCCTCT
ATTTTCGAGGTGGTGGATCAGAAGCCCGACATCGAAGGCAAGCTGTTTTT
CCCAATCAAGGGCACCGAGCTGTATGCCGTGCACAGAGCCAGCTTCAACA
TCAAGCTGCCCGGCGAGACACTGGTCAAGAGCAGAGAAGTGCTGCGGAAG
GCCAGAGAGGACAATCTGAAACTGATGAACCAGAAGCTCAACTTCCTGCG
GAACGTGCTGCACTTCCAGCAGTTCGAGGACATCACCGAGAGAGAGAAGC
GGGTCACCAAGTGGATCAGCAGACAAGAGAACAGCGACGTGCCCCTGGTG
TACCAGGATGAGCTGATCCAGATCCGCGAGCTGATGTACAAGCCTTACAA
GGACTGGGTCGCCTTCCTGAAGCAGCTCCACAAGAGACTGGAAGTCGAGA
TCGGCAAAGAAGTGAAGCACTGGCGGAAGTCCCTGAGCGACGGAAGAAAG
GGCCTGTACGGCATCTCCCTGAAGAACATCGACGAGATCGATCGGACCCG
GAAGTTCCTGCTGAGATGGTCCCTGAGGCCTACCGAACCTGGCGAAGTGC
GTAGACTGGAACCCGGCCAGAGATTCGCCATCGACCAGCTGAATCACCTG
AACGCCCTGAAAGAAGATCGGCTGAAGAAGATGGCCAACACCATCATCAT
GCACGCCCTGGGCTACTGCTACGACGTGCGGAAGAAGAAATGGCAGGCTA
AGAACCCCGCCTGCCAGATCATCCTGTTCGAGGATCTGAGCAACTACAAC
CCCTACGAGGAAAGGTCCCGCTTCGAGAACAGCAAGCTCATGAAGTGGTC
CAGACGCGAGATCCCCAGACAGGTTGCACTGCAGGGCGAGATCTATGGCC
TGCAAGTGGGAGAAGTGGGCGCTCAGTTCAGCAGCAGATTCCACGCCAAG
ACAGGCAGCCCTGGCATCAGATGTAGCGTCGTGACCAAAGAGAAGCTGCA
GGACAATCGGTTCTTCAAGAATCTGCAGAGAGAGGGCAGACTGACCCTGG
ACAAAATCGCCGTGCTGAAAGAGGGCGATCTGTACCCAGACAAAGGCGGC
GAGAAGTTCATCAGCCTGAGCAAGGATCGGAAGTGCGTGACCACACACGC
CGACATCAACGCCGCTCAGAACCTGCAGAAGCGGTTCTGGACAAGAACCC
ACGGCTTCTACAAGGTGTACTGCAAGGCCTACCAGGTGGACGGCCAGACC
GTGTACATCCCTGAGAGCAAGGACCAGAAGCAGAAGATCATCGAAGAGTT
CGGCGAGGGCTACTTCATTCTGAAGGACGGGGTGTACGAATGGGTCAACG
CCGGCAAGCTGAAAATCAAGAAGGGCAGCTCCAAGCAGAGCAGCAGCGAG
CTGGTGGATAGCGACATCCTGAAAGACAGCTTCGACCTGGCCTCCGAGCT
GAAAGGCGAAAAGCTGATGCTGTACAGGGACCCCAGCGGCAATGTGTTCC
CCAGCGACAAATGGATGGCCGCTGGCGTGTTCTTCGGAAAGCTGGAACGC
ATCCTGATCAGCAAGCTGACCAACCAGTACTCCATCAGCACCATCGAGGA
CGACAGCAGCAAGCAGTCTATGAAAAGGCCGGCGGCCACGAAAAAGGCCG
GCCAGGCAAAAAAGAAAAAG

Fusion proteins comprising a Cas9 domain and a Cytidine Deaminase or Adenosine Deaminase

Some aspects of the disclosure provide fusion proteins comprising a Cas9 domain or other nucleic acid programmable DNA binding protein and one or more cytidine deaminase or adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases provided herein. For example, and without limitation, in some embodiments, the fusion protein comprises the structure:

NH₂-[cytidine deaminase]-[Cas9 domain]-COOH; or

NH₂-[Cas9 domain]-[cytidine deaminase]-COOH.

In some embodiments, the fusion proteins comprising a cytidine deaminase or adenosine deaminase and a napDNAbp (e.g., Cas9 domain) do not include a linker sequence. In some embodiments, a linker is present between the cytidine or adenosine deaminase and the napDNAbp. In some embodiments, the “-” used in the general architecture above indicates the presence of an optional linker. In some embodiments, cytidine or adenosine deaminase and the napDNAbp are fused via any of the linkers provided herein. For example, in some embodiments the cytidine or adenosine deaminase and the napDNAbp are fused via any of the linkers in the section entitled “Linkers”.

### Fusion Proteins Comprising a Nuclear Localization Sequence (NLS)

In some embodiments, the fusion proteins provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, any of the fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the Cas9 domain. In some embodiments, the NLS is fused to the C-terminus of the Cas9 domain. In some embodiments, the NLS is fused to the N-terminus of the cytidine or adenosine deaminase. In some embodiments, the NLS is fused to the C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, an NLS comprises the amino acid sequence KRTADGSEFESPKKKRKV, KRPAATKKAGQAKKKK, KKTELQTTNAENKTKKL, KRGINDRNFWRGENGRKTR, RKSGKIAAIVVKRPRKPKKKRKV, or MDSLLMNRRKFLYQFKNVRWAKGRRETYLC.

In some embodiments, the general architecture of exemplary Cas9 fusion proteins with a cytidine or adenosine deaminase and a Cas9 domain comprises any one of the following structures, where NLS is a nuclear localization sequence (e.g., any NLS provided herein), NH₂ is the N-terminus of the fusion protein, and COOH is the C-terminus of the fusion protein:

NH₂—NLS-[cytidine deaminase]-[Cas9 domain]-COOH;

NH₂—NLS [Cas9 domain]-[cytidine deaminase]-COOH;

NH₂-[cytidine deaminase]-[Cas9 domain]-NLS—COOH; or

NH₂-[Cas9 domain]-[cytidine deaminase]-NLS—COOH.

NH₂—NLS-[adenosine deaminase]-[Cas9 domain]-COOH;

NH₂-NLS [Cas9 domain]-[adenosine deaminase]-COOH;

NH₂-[adenosine deaminase]-[Cas9 domain]-NLS—COOH; or

NH₂-[Cas9 domain]-[adenosine deaminase]-NLS—COOH.

In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR[PAATKKAGQA]KKKK, is the prototype of the ubiquitous bipartite signal: two clusters of basic amino acids, separated by a spacer of about 10 amino acids.

The sequence of an exemplary bipartite NLS follows: PKKKRKVEGADKRTADGSEFES PKKKRKV

In some embodiments, the fusion proteins comprising a cytidine or adenosine deaminase, a Cas9 domain, and an NLS do not comprise a linker sequence. In some embodiments, linker sequences between one or more of the domains or proteins (e.g., cytidine or adenosine deaminase, Cas9 domain or NLS) are present.

It should be appreciated that the fusion proteins of the present disclosure may comprise one or more additional features. For example, in some embodiments, the fusion protein may comprise inhibitors, cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

### Linkers

In certain embodiments, linkers may be used to link any of the peptides or peptide domains of the invention. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is an amino acid or a plurality of amino acids (e.g., a peptide or protein). In some embodiments, the linker is a bond (e.g., a covalent bond), an organic molecule, group, polymer, or chemical moiety. In some embodiments, the cytidine or adenosine deaminase and the napDNAbp are fused via a linker that comprises 4, 16, 32, or 104 amino acids in length. In some embodiments, the linker is about 3 to about 104 amino acids in length. In some embodiments, any of the fusion proteins provided herein, comprise a cytidine or adenosine deaminase and a Cas9 domain that are fused to each other via a linker. e.g., Various linker lengths and flexibilities between the cytidine or adenosine deaminase and the Cas9 domain can be employed (e.g., ranging from very flexible linkers of the form (GGGS)ₙ, (GGGGS)ₙ, and (G)ₙ to more rigid linkers of the form (EAAAK)ₙ, (SGGS)ₙ, SGSETPGTSESATPES (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference) and (XP)ₙ) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)ₙ motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES.

Cas9 Complexes with Guide RNAs

Some aspects of this disclosure provide complexes comprising any of the fusion proteins provided herein, and a guide RNA bound to a Cas9 domain (e.g., a dCas9, a nuclease active Cas9, or a Cas9 nickase) of fusion protein. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is an RNA sequence. In some embodiments, the target sequence is a sequence in the genome of a mammal. In some embodiments, the target sequence is a sequence in the genome of a human. In some embodiments, the 3′ end of the target sequence is immediately adjacent to a canonical PAM sequence (NGG). In some embodiments, the guide nucleic acid (e.g., guide RNA) is complementary to a sequence associated with a disease or disorder.

In some embodiments, the guide RNA is designed to disrupt a splice site (i.e., a splice acceptor (SA) or a splice donor (SD). In some embodiments, the guide RNA is designed such that the base editing results in a premature STOP codon. Tables 8A Table 8B and Table 8C provide a nonexhaustive list of gRNA target sequences designed to disrupt a splice site or to result in a premature STOP codon.

Provided herein are compositions and methods for base editing in host cells, e.g. immune cells. Further provided herein are compositions comprising a guide polynucleic acid sequence, e.g. a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g. a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in an immune cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

TABLE 8A
gRNAs: Splice Site and STOP Codons

| | | | gRNA |
|---|---|---|---|
| | | Targeting | Spacer |
| Gene | Description | sequence | Sequence |
| VISTA | Exon 1 SD | CCTTACCTAG | CCUUACCUAG |
| | (pos6) | GGACGCAGCC | GGACGCAGCC |
| | Exon 1 STOP | GGATCCCCAG | GGAUCCCCAG |
| | (pos7) | CGCCAGCTGC | CGCCAGCUGC |
| | Exon 1 STOP | AGCGCCAGCT | AGCGCCAGCU |
| | (pos5) | GCCGGCCTCC | GCCGGCCUCC |
| | Exon 1 STOP | GCGCCAGCTG | GCGCCAGCUG |
| | (pos4) | CCGGCCTCCA | CCGGCCUCCA |
| | Exon 2 STOP | CCTGGCTCAG | CCUGGCUCAG |
| | (pos8) | CGCCACGGGC | CGCCACGGGC |
| | Exon 2 STOP | GCTGCAGGTG | GCUGCAGGUG |
| | (pos5) | CAGACAGGTG | CAGACAGGUG |
| | Exon 2 STOP | GCGGTACCAC | GCGGUACCAC |
| | (pos7) | GTCTTGTAGA | GUCUUGUAGA |
| | Exon 3 SA | TGCCTGTGGG | UGCCUGUGGG |
| | (pos4) | AACAAACAGA | AACAAACAGA |
| | Exon 3 SD | CTTACTTTCA | CUUACUUUCA |
| | (pos5) | CTATCCTGGG | CUAUCCUGGG |
| | Exon 3 SD | TCCCTTACTT | UCCCUUACUU |
| | (pos8) | TCACTATCCT | UCACUAUCCU |
| | Exon 3 STOP | CTCCCAGGAT | CUCCCAGGAU |
| | (pos5) | AGTGAAAGTA | AGUGAAAGUA |
| | Exon 4 SA | TGATGTCTGA | UGAUGUCUGA |
| | (pos7) | AAGGGCAGAG | AAGGGCAGAG |
| | Exon 5 STOP | TGCCCAGGAG | UGCCCAGGAG |
| | (pos5) | CTGGTGCGGA | CUGGUGCGGA |
| | Exon 6 SA | TTGCTGCCAC | UUGCUGCCAC |
| | (pos4) | AGAACCAGAA | AGAACCAGAA |
| | Exon 6 STOP | ATTCAAGGGA | AUUCAAGGGA |
| | (pos4) | TTGAAAACCC | UUGAAAACCC |
| | Exon 6 STOP | ACCTGCCCAG | ACCUGCCCAG |
| | (pos8) | GGGATACCCG | GGGAUACCCG |
| | Exon 6 STOP | CAGCGGCAGC | CAGCGGCAGC |
| | (pos7) | CTTCTGAGTC | CUUCUGAGUC |
| TRAC | Exon 1 STOP 1 | GCTACAAACA | GCUACAAACA |
| | (pos5) | AGCTCATCTT | AGCUCAUCUU |
| | Exon 1 STOP 2 | CCAGCCAAGT | CCAGCCAAGU |
| | (pos6) | ACGTAAGTAG | ACGUAAGUAG |
| | Exon 1 SA | CTGGATATCT | CUGGAUAUCU |
| | (pos9) | GTGGGACAAG | GUGGGACAAG |
| | Exon 1 SD | CTTACCTGGG | CUUACCUGGG |
| | | CTGGGGAAGA | CUGGGGAAGA |
| | Exon 3SA | TTCGTATCTG | UUCGUAUCUG |
| | | TAAAACCAAG | UAAAACCAAG |
| | Exon 3 STOP | TTTCAAAACC | UUUCAAAACC |
| | | TGTCAGTGAT | UGUCAGUGAU |
| | Exon 3 STOP | TTCAAAACCT | UUCAAAACCU |
| | | GTCAGTGATT | GUCAGUGAUU |
| Tim-3 | Exon 2 SA | GGACCCTGCA | GGACCCUGCA |
| | (pos6) | TAGAGAGAGA | UAGAGAGAGA |
| | Exon 2 STOP | TGCCCCAGCA | UGCCCCAGCA |
| | (pos5) | GACGGGCACG | GACGGGCACG |
| | Exon 3 SD | GTTACCTGGG | GUUACCUGGG |
| | (pos5) | CCATGTCCCC | CCAUGUCCCC |
| | Exon 4 SD | CTTACTGTTA | CUUACUGUUA |
| | (pos5) | GATTTATATC | GAUUUAUAUC |
| | Exon 4 SD | TTACTGTTAG | UUACUGUUAG |
| | (pos4) | ATTTATATCA | AUUUAUAUCA |
| | Exon 5 SA | TTTGCTATGG | UUUGCUAUGG |
| | (pos5) | AAACACAAAC | AAACACAAAC |
| | Exon 5 STOP | TCCATAGCAA | UCCAUAGCAA |
| | (pos8) | ATATCCACAT | AUAUCCACAU |
| | Exon 7 STOP | GCAGCAACCC | GCAGCAACCC |
| | (pos5) | TCACAACCTT | UCACAACCUU |
| | Exon 7 STOP | CAGCAACCCT | CAGCAACCCU |
| | (pos 4) | CACAACCTTT | CACAACCUUU |
| TIGIT | Exon 1 STOP | AGGCAGGCTC | AGGCAGGCUC |
| | (pos4) | CCCTCGCCTC | CCCUCGCCUC |
| | Exon 2 STOP | GGAGCAGCAG | GGAGCAGCAG |
| | (5&8) | GACCAGCTTC | GACCAGCUUC |
| | Exon 2 SD | CAGGAATACC | CAGGAAUACC |
| | (pos9) | TGAGCTTTCT | UGAGCUUUCU |
| | Exon 3 STOP | AGGTTCCAGA | AGGUUCCAGA |
| | (pos7) | TTCCATTGCT | UUCCAUUGCU |
| | Exon 1 STOP | CTGGGCCCAG | CUGGGCCCAG |
| | | GGGCTGAGGC | GGGCUGAGGC |
| | Exon 2 STOP | GATCGAGTGG | GAUCGAGUGG |
| | | CCCCAGGTCC | CCCCAGGUCC |
| TGFbRII | Exon 1 SD | TCACCCGACT | UCACCCGACU |
| | (JMG79) | TCTGAACGTG | UCUGAACGUG |
| | Exon 3 SD | TTACCTGCCC | UUACCUGCCC |
| | (JMG83) | ACTGTTAGCC | ACUGUUAGCC |
| | Exon 2 STOP | GAAGCCACAG | GAAGCCACAG |
| | (JMG80) | GAAGTCTGTG | GAAGUCUGUG |
| | Exon 3 STOP | ACTCCAGTTC | ACUCCAGUUC |
| | (JMG81) | CTGACGGCTG | CUGACGGCUG |
| | Exon 3 STOP | ACCTACAGGA | ACCUACAGGA |
| | (JMG82) | GTACCTGACG | GUACCUGACG |
| | Exon 4 STOP | TTCCCAGAGC | UUCCCAGAGC |
| | (JMG84) | ACCAGAGCCA | ACCAGAGCCA |
| | Exon 1 STOP | ACGTTCAGAA | ACGUUCAGAA |
| | (JMG85) | GTCGGGTGAG | GUCGGGUGAG |
| | Exon 3 STOP | TTCAGAGCAG | UUCAGAGCAG |
| | (pos8) | TTTGAGACAG | UUUGAGACAG |
| | Exon 1 SD | TCACCCGACT | UCACCCGACU |
| | | TCTGAACGTG | UCUGAACGUG |
| | Exon 1 Stop | ACGTTCAGAA | ACGUUCAGAA |
| | | GTCGGGTGAG | GUCGGGUGAG |
| | Exon 2 SD 1 | TTTACTATGT | UUUACUAUGU |
| | | CTCAGTGGAT | CUCAGUGGAU |
| | Exon 2 SD2 | CTTTACTATG | CUUUACUAUG |
| | | TCTCAGTGGA | UCUCAGUGGA |
| | Exon 3 STOP | GAAGCCACAG | GAAGCCACAG |
| | | GAAGTCTGTG | GAAGUCUGU |
| | | | G |
| | Exon 6 SD | TTACCTGCCC | UUACCUGCCC |
| | | ACTGTTAGCC | ACUGUUAGCC |
| | Exon 6 STOP 1 | TTCAGAGCAG | UUCAGAGCAG |
| | | TTTGAGACAG | UUUGAGACAG |
| | Exon 6 STOP 2 | ACTCCAGTTC | ACUCCAGUUC |
| | | CTGACGGCTG | CUGACGGCUG |
| | Exon 6 STOP | ACCTACAGGA | ACCUACAGGA |
| | | GTACCTGACG | GUACCUGACG |
| | Exon 7 STOP | TTCCCAGAGC | UUCCCAGAGC |
| | | ACCAGAGCCA | ACCAGAGCCA |
| | Exon 8 STOP | AGCCAGAAGC | AGCCAGAAGC |
| | | TGGGAATTTC | UGGGAAUUUC |
| | Isoform ATG | TATCATGTCG | UAUCAUGUCG |
| | | TTATTAACTG | UUAUUAACUG |
| RFXANK | Exon 2 SA | CCTGCTGGGA | CCUGCUGGGA |
| | (JMG8) | AACAGACAAC | AACAGACAAC |
| | Exon 2 SD | CACTCACAGT | CACUCACAGU |
| | (JMG9) | CTAGGGTGGC | CUAGGGUGGC |
| | Exon 2 STOP | CAACCGGCAG | CAACCGGCAG |
| | (pos8) | CGAGGGAACG | CGAGGGAACG |
| | Exon 3 SA | ACAGGGCTGG | ACAGGGCUGG |
| | (pos7) | GGCAGGACAG | GGCAGGACAG |
| | Exon 3 STOP | CATCCACCAG | CAUCCACCAG |
| | (pos8) | CTCGCAGCAC | CUCGCAGCAC |
| | Exon 3 STOP | ATCCACCAGC | AUCCACCAGC |
| | (pos7) | TCGCAGCACA | UCGCAGCACA |
| | Exon 3 STOP | TCCACCAGCT | UCCACCAGCU |
| | (pos6) | CGCAGCACAG | CGCAGCACAG |
| | Exon 3 STOP | CCACCAGCTC | CCACCAGCUC |
| | (pos5) | GCAGCACAGG | GCAGCACAGG |
| | Exon 4 SA | TGTCACCTGG | UGUCACCUGG |
| | (JMG10) | CAGGAGGAGG | CAGGAGGAGG |
| | Exon 4 SA | GTCACCTGGC | GUCACCUGGC |
| | (pos6) | AGGAGGAGGC | AGGAGGAGGC |
| | Exon 5 SA | GGCACCCTGC | GGCACCCUGC |
| | (pos7) | AGGGAGAAGA | AGGGAGAAGA |
| | Exon 5 SA | GCACCCTGCA | GCACCCUGCA |
| | (JMG11) | GGGAGAAGAA | GGGAGAAGAA |
| | Exon 6 SA | ATTCTGTCGT | AUUCUGUCGU |
| | (pos4) | GGGTAGGGGC | GGGUAGGGGC |
| | Exon 6 SA | CTCCATTCTG | CUCCAUUCUG |
| | (JMG12) | TCGTGGGTAG | UCGUGGGUAG |
| | Exon 7 SA | CCTCGGGCTG | CCUCGGGCUG |
| | (pos8) | CAAAGGAGAG | CAAAGGAGAG |
| | Exon 7 SA | CGGGCTGCAA | CGGGCUGCAA |
| | (pos5) | AGGAGAGGGG | AGGAGAGGGG |
| | Exon 7 SD | GCTGACCTTT | GCUGACCUUU |
| | (pos6) | CCGGTATCCC | CCGGUAUCCC |
| | Exon 7 SD | CTGACCTTTC | CUGACCUUUC |
| | (pos5) | CGGTATCCCA | CGGUAUCCCA |
| | Exon 8 SA | TGTTGCACTG | UGUUGCACUG |
| | (pos8) | AGATGGGGCA | AGAUGGGGCA |
| | Exon 8 SA | CTGTTGCACT | CUGUUGCACU |
| | (pos9) | GAGATGGGGC | GAGAUGGGGC |
| PVRIG | Exon 1 STOP | GCCCTGCAGC | GCCCUGCAGC |
| (CD112 | (pos7) | CCCCAGAACC | CCCCAGAACC |
| R) | | | |
| | Exon 1 SD | CTCACCCGCA | CUCACCCGCA |
| | (pos5) | GTGACACACA | GUGACACACA |
| | Exon 1 STOP | GCAGCACCCA | GCAGCACCCA |
| | (pos8) | GGGCAGGACC | GGGCAGGACC |
| | Exon 1 STOP | CAGCACCCAG | CAGCACCCAG |
| | (pos7) | GGCAGGACCA | GGCAGGACCA |
| | Exon 2 SA | GTCCCTGTGG | GUCCCUGUGG |
| | (pos5) | AACAGCAGCA | AACAGCAGCA |
| | Exon 2 STOP | GTGGGTTCAA | GUGGGUUCAA |
| | (pos8) | GTTCGGATGG | GUUCGGAUGG |
| | Exon 2 SD | GCCCCACCTG | GCCCCACCUG |
| | (pos 7) | GGTCTGAGCT | GGUCUGAGCU |
| | Exon 2 SD | GGCCCCACCT | GGCCCCACCU |
| | (pos8) | GGGTCTGAGC | GGGUCUGAGC |
| | Exon 2 SD | CCACCTGGGT | CCACCUGGGU |
| | (pos4) | CTGAGCTGGG | CUGAGCUGGG |
| | Exon 2 STOP | AGGCCTCCCA | AGGCCUCCCA |
| | (pos8) | GGAGCCCTCA | GGAGCCCUCA |
| | Exon 2 STOP | CTCCCAGGAG | CUCCCAGGAG |
| | (pos4) | CCCTCAGGGA | CCCUCAGGGA |
| | Exon 2 STOP | CCCCCAGCTC | CCCCCAGCUC |
| | (pos4) | ACAGTCACCA | ACAGUCACCA |
| | Exon 3 SD | GGTCTCACCG | GGUCUCACCG |
| | (pos8) | GTGCTTATGT | GUGCUUAUGU |
| | Exon 3 STOP | TGCTGCGCCG | UGCUGCGCCG |
| | (pos9) | ACATAAGCAC | ACAUAAGCAC |
| | Exon 4 SA | GGCAGGGCTG | GGCAGGGCUG |
| | (pos8) | GGAGAGAGCA | GGAGAGAGCA |
| | Exon 4 STOP | CGAGAGCACG | CGAGAGCACG |
| | (pos9) | AGCATGGGTG | AGCAUGGGUG |
| | Exon 4 STOP | GAGCACGAGC | GAGCACGAGC |
| | (pos6) | ATGGGTGAGG | AUGGGUGAGG |
| | Exon 4 STOP | AGCACGAGCA | AGCACGAGCA |
| | (pos5) | TGGGTGAGGA | UGGGUGAGGA |
| | Exon 4 STOP | GCACGAGCAT | GCACGAGCAU |
| | (pos4) | GGGTGAGGAG | GGGUGAGGAG |
| | Exon 4 SD | CTCACCCATG | CUCACCCAUG |
| | (pos5) | CTCGTGCTCT | CUCGUGCUCU |
| | Exon 5 SA | GGTGCCTGCG | GGUGCCUGCG |
| | (pos6) | CGGGGGAAGG | CGGGGGAAGG |
| | Exon 5 SA | GTGCCTGCGC | GUGCCUGCGC |
| | (pos5) | GGGGGAAGGA | GGGGGAAGGA |
| | Exon 5 SA | CTTGGTGCCT | CUUGGUGCCU |
| | (pos9) | GCGCGGGGGA | GCGCGGGGGA |
| | Exon 5 STOP | GGCCCCAGGG | GGCCCCAGGG |
| | (pos6) | CCCTGCCGCC | CCCUGCCGCC |
| | Exon 5 STOP | TCTACGCTCA | UCUACGCUCA |
| | (pos9) | GGCAGGGGAG | GGCAGGGGAG |
| | Exon 5 STOP | CCACCAGGAC | CCACCAGGAC |
| | (pos4) | GGCCCCCCAT | GGCCCCCCAU |
| | Exon 5 STOP | AGGCCCAGGC | AGGCCCAGGC |
| | (pos5) | GGCAGGGCCC | GGCAGGGCCC |
| | Exon 5 STOP | GGCCCAGGCG | GGCCCAGGCG |
| | (pos4) | GCAGGGCCCT | GCAGGGCCCU |
| PDCD1 | Exon 1 STOP 2 | ACGACTGGCC | ACGACUGGCC |
| | (pos9) | AGGGCGCCTG | AGGGCGCCUG |
| | Exon 1 STOP 4 | CACCGCCCAG | CACCGCCCAG |
| | (pos7) | ACGACTGGCC | ACGACUGGCC |
| | Exon 1 STOP | CTACAACTGG | CUACAACUGG |
| | (pos4) | GCTGGCGGCC | GCUGGCGGCC |
| | Exon 1 SD | CACCTACCTA | CACCUACCUA |
| | | AGAACCATCC | AGAACCAUCC |
| | Exon 2 SA | GGAGTCTGAG | GGAGUCUGAG |
| | | AGATGGAGAG | AGAUGGAGAG |
| | Exon 2 STOP 1 | CAGCAACCAG | CAGCAACCAG |
| | (pos8) | ACGGACAAGC | ACGGACAAGC |
| | Exon 2 STOP 2 | GTGTCACACA | GUGUCACACA |
| | (pos9) | ACTGCCCAAC | ACUGCCCAAC |
| | Exon 3 STOP 1 | AGCCGGCCAG | AGCCGGCCAG |
| | (pos8) | TTCCAAACCC | UUCCAAACCC |
| | Exon 3 STOP | CAGTTCCAAA | CAGUUCCAAA |
| | (pos7) | CCCTGGTGGT | CCCUGGUGGU |
| | Exon 3 STOP 2 | CGGCCAGTTC | CGGCCAGUUC |
| | (pos5) | CAAACCCTGG | CAAACCCUGG |
| | Exon 3 STOP | GGACCCAGAC | GGACCCAGAC |
| | (pos5) | TAGCAGCACC | UAGCAGCACC |
| | Exon 3 SD | GACGTTACCT | GACGUUACCU |
| | | CGTGCGGCCC | CGUGCGGCCC |
| | Exon 4 SA | TCCCTGCAGA | UCCCUGCAGA |
| | | GAAACACACT | GAAACACACU |
| | Exon 4 SD | GAGACTCACC | GAGACUCACC |
| | | AGGGGCTGGC | AGGGGCUGGC |
| | Exon 5 SA | CCTCCTTCTT | CCUCCUUCUU |
| | | TGAGGAGAAA | UGAGGAGAAA |
| | Exon 2 STOP | GGGGTTCCAG | GGGGUUCCAG |
| | (pos 7) | GGCCTGTCTG | GGCCUGUCUG |
| | Exon 3 SA | TTCTCTCTGG | UUCUCUCUGG |
| | | AAGGGCACAA | AAGGGCACAA |
| | Exon 5 STOP 1 | CCAGTGGCGA | CCAGUGGCGA |
| | (pos 8) | GAGAAGACCC | GAGAAGACCC |
| | Exon 5 STOP 2 | TGCCCAGCCA | UGCCCAGCCA |
| | (pos 5) | CTGAGGCCTG | CUGAGGCCUG |
| | Exon 1 STOP 1 | CGACTGGCCA | CGACUGGCCA |
| | (pos8) | GGGCGCCTGT | GGGCGCCUGU |
| | Exon 1 STOP 3 | ACCGCCCAGA | ACCGCCCAGA |
| | (pos6) | CGACTGGCCA | CGACUGGCCA |
| Lag3 | Exon 1 STOP | GTTTCTGCAG | GUUUCUGCAG |
| | (pos8) | CCGCTTTGGG | CCGCUUUGGG |
| | Exon 1 SD | TTACCTGGAG | UUACCUGGAG |
| | (pos4) | CCACCCAAAG | CCACCCAAAG |
| | Exon 2 SA | TCACTAGGTG | UCACUAGGUG |
| | (pos4) | AGCAAAAGAG | AGCAAAAGAG |
| | Exon 2 STOP | GCCTCTCCAG | GCCUCUCCAG |
| | (pos8) | CCAGGGGCTG | CCAGGGGCUG |
| | Exon 2 STOP | CTTGGCAGCA | CUUGGCAGCA |
| | (pos 6) | TCAGCCAGAC | UCAGCCAGAC |
| | Exon 3 SA | CCACTGGGCG | CCACUGGGCG |
| | (pos4) | GGAAAGAGAA | GGAAAGAGAA |
| | Exon 3 SD | ACATACTCGA | ACAUACUCGA |
| | (pos6) | GGCCTGGCCC | GGCCUGGCCC |
| | Exon 3 STOP | CCTGCAGCCC | CCUGCAGCCC |
| | (pos5) | CGCGTCCAGC | CGCGUCCAGC |
| | Exon 3 STOP | CGCGTCCAGC | CGCGUCCAGC |
| | (pos7) | TGGATGAGCG | UGGAUGAGCG |
| | Exon 3 STOP | TGGGCCAGGC | UGGGCCAGGC |
| | (pos6) | CTCGAGTATG | CUCGAGUAUG |
| | Exon 4 SD | GGGAGTTACC | GGGAGUUACC |
| | (pos4) | CAGAACAGTG | CAGAACAGUG |
| | Exon 4 STOP | CCTGCCCCAA | CCUGCCCCAA |
| | (pos8) | GTCAGCCCCA | GUCAGCCCCA |
| | Exon 4 STOP | GCCAGGGCCG | GCCAGGGCCG |
| | (pos9) | AGTCCCTGTC | AGUCCCUGUC |
| | Exon 4 STOP | CCAGGGCCGA | CCAGGGCCGA |
| | (pos8) | GTCCCTGTCC | GUCCCUGUCC |
| | Exon 4 STOP | GCCCCAGGGC | GCCCCAGGGC |
| | (pos4) | CCAGAGTCCA | CCAGAGUCCA |
| | Exon 5 STOP | ATGTGAGCCA | AUGUGAGCCA |
| | (pos9) | GGCCCAGGCT | GGCCCAGGCU |
| | Exon 5 STOP | GAGGAGTCCA | GAGGAGUCCA |
| | (pos 8) | CTTGGCAGTG | CUUGGCAGUG |
| | Exon 6 SA | GAGTCACTGA | GAGUCACUGA |
| | (pos7) | AAAGAGTAGA | AAAGAGUAGA |
| | Exon 6 STOP | CTGGACAAGA | CUGGACAAGA |
| | (pos6) | ACGCTTTGTG | ACGCUUUGUG |
| | Exon 6 STOP | CCATCCCAGA | CCAUCCCAGA |
| | (pos7) | GGAGTTTCTC | GGAGUUUCUC |
| | Exon 6 STOP | TGGCAATGCC | UGGCAAUGCC |
| | (pos4) | AGCTGTACCA | AGCUGUACCA |
| | Exon 6 STOP | TACCAGGGGG | UACCAGGGGG |
| | (pos4) | AGAGGCTTCT | AGAGGCUUCU |
| | Exon 6 STOP | GGCATTGCCA | GGCAUUGCCA |
| | (pos8) | AGGCTGGGAA | AGGCUGGGAA |
| | Exon 7 SA | GGCACCTATG | GGCACCUAUG |
| | (pos6) | GAGAAAGTAC | GAGAAAGUAC |
| | Exon 7 STOP | AGACAGGTGA | AGACAGGUGA |
| | (pos4) | GCCAGGGACA | GCCAGGGACA |
| | Exon 7 SD | GGCTCACCTG | GGCUCACCUG |
| | (pos7) | TCTTCTCCAA | UCUUCUCCAA |
| | Exon 8 SA | GTCGCCACTG | GUCGCCACUG |
| | (pos8) | TGAGAAGAGA | UGAGAAGAGA |
| | Exon 8 STOP | GCAGGCTCAG | GCAGGCUCAG |
| | (pos8) | AGCAAGATAG | AGCAAGAUAG |
| | Exon 8 STOP | GCTGGAGCAA | GCUGGAGCAA |
| | (pos8) | GAACCGGAGC | GAACCGGAGC |
| CTLA-4 | Exon 1 SD | ACTCACCTTT | ACUCACCUUU |
| | (pos 6) | GCAGAAGACA | GCAGAAGACA |
| | Exon 1 SD | CACTCACCTT | CACUCACCUU |
| | | TGCAGAAGAC | UGCAGAAGAC |
| | Exon 1 STOP | AGGGCCAGGT | AGGGCCAGGU |
| | (pos5) | CCTGGTAGCC | CCUGGUAGCC |
| | Exon 2 STOP | GGCCCAGCCT | GGCCCAGCCU |
| | | GCTGTGGTAC | GCUGUGGUAC |
| | Exon 2 STOP | GCTTCGGCAG | GCUUCGGCAG |
| | (pos 8) | GCTGACAGCC | GCUGACAGCC |
| | Exon 2 STOP | TATCCAAGGA | UAUCCAAGGA |
| | | CTGAGGGCCA | CUGAGGGCCA |
| | Exon 2 STOP | GGAACCCAGA | GGAACCCAGA |
| | | TTTATGTAAT | UUUAUGUAAU |
| | Exon 2 SD | GCTCACCAAT | GCUCACCAAU |
| | | TACATAAATC | UACAUAAAUC |
| | Exon 2 SD | CTCACCAATT | CUCACCAAUU |
| | | ACATAAATCT | ACAUAAAUCU |
| | Exon 1 STOP | CTCAGCTGAA | CUCAGCUGAA |
| | | CCTGGCTACC | CCUGGCUACC |
| Chi3l1 | Exon 1 STOP | GGCGTCTCAA | GGCGUCUCAA |
| | (pos8) | ACAGGTATCT | ACAGGUAUCU |
| | Exon 1 SA | CAAAGCCTGA | CAAAGCCUGA |
| | (pos7) | AGAGAAATCC | AGAGAAAUCC |
| | Exon 3 SA | AGAGCCTGAA | AGAGCCUGAA |
| | (pos6) | GGAGAAGTCT | GGAGAAGUCU |
| | Exon 3 STOP | TCCCAGTACC | UCCCAGUACC |
| | (pos4) | GGGAAGGCGA | GGGAAGGCGA |
| | Exon 4 SA | GGTTCCTGTG | GGUUCCUGUG |
| | (pos6) | GAGCACAGGG | GAGCACAGGG |
| | Exon 4 SA | TGGGGTTCCT | UGGGGUUCCU |
| | (pos9) | GTGGAGCACA | GUGGAGCACA |
| | Exon 6 SA | TCATTTCCTA | UCAUUUCCUA |
| | (pos8) | GATGGGAGAC | GAUGGGAGAC |
| | Exon 6 SA | TTCCTAGATG | UUCCUAGAUG |
| | (pos4) | GGAGACAGGC | GGAGACAGGC |
| | Exon 8 SA | CCAGGTGTCT | CCAGGUGUCU |
| | (pos9) | GAGGAGGAAG | GAGGAGGAAG |
| | Exon 8 SA | GTGTCTGAGG | GUGUCUGAGG |
| | (pos5) | AGGAAGGGGA | AGGAAGGGGA |
| | Exon 9 SA | TAGTCCTGGG | UAGUCCUGGG |
| | (pos6) | TGGGGTAGGG | UGGGGUAGGG |
| | Exon 9 SA | AGTCCTGGGT | AGUCCUGGGU |
| | (pos5) | GGGGTAGGGT | GGGGUAGGGU |
| | Exon 9 SD | CATTACCTCA | CAUUACCUCA |
| | (pos6) | TAGTAGGCAA | UAGUAGGCAA |
| | Exon 9 SD | CCATTACCTC | CCAUUACCUC |
| | (pos7) | ATAGTAGGCA | AUAGUAGGCA |
| | Exon 10 SA | ACAGATCTGA | ACAGAUCUGA |
| | (pos7) | GCAGATAACA | GCAGAUAACA |
| | Exon 10 STOP | TCCTACCCAC | UCCUACCCAC |
| | (pos 7) | TGGTTGCCCT | UGGUUGCCCU |
| | Exon 11 STOP | AGGTGCAGTA | AGGUGCAGUA |
| | (pos7) | CCTGAAGGAC | CCUGAAGGAC |
| | Exon 11 STOP | CAGGCAGCTG | CAGGCAGCUG |
| | (pos5) | GCGGGCGCCA | GCGGGCGCCA |
| | Exon 11 STOP | GACTTCCAGG | GACUUCCAGG |
| | (pos7) | GCTCCTTCTG | GCUCCUUCUG |
| CD96 | Exon 1 STOP | CATCCAGATA | CAUCCAGAUA |
| | (pos5) | CATTTTGTCA | CAUUUUGUCA |
| | Exon 2 STOP | ACCTGCCAAA | ACCUGCCAAA |
| | (pos5) | CACAGACAGT | CACAGACAGU |
| | Exon 2 STOP | CGTGCAGATG | CGUGCAGAUG |
| | (pos7) | CAATGGTCCA | CAAUGGUCCA |
| | Exon 3 SA | TGTAACTGTA | UGUAACUGUA |
| | (pos6) | ACAAAACATA | ACAAAACAUA |
| | Exon 3 SD | ACTTACCACC | ACUUACCACC |
| | (pos6) | GACCATGCAT | GACCAUGCAU |
| | Exon 5 SD | CTTACCAAAA | CUUACCAAAA |
| | (pos5) | ACCTTGACTG | ACCUUGACUG |
| | Exon 5 STOP | CCAGTCCAAA | CCAGUCCAAA |
| | (pos6) | TCTTCGATGA | UCUUCGAUGA |
| | Exon 5 STOP | CAGTCCAAAT | CAGUCCAAAU |
| | (pos7) | CTTCGATGAT | CUUCGAUGAU |
| | Exon 7 STOP | AAACCATGTG | AAACCAUGUG |
| | (pos4) | ATATTTGCTT | AUAUUUGCUU |
| | Exon 8 STOP | ATGTTCCACA | AUGUUCCACA |
| | (pos6) | CTTTATTTCC | CUUUAUUUCC |
| | Exon 10 SD | TCACGTTGAG | UCACGUUGAG |
| | (pos4) | GAGTGGTGTT | GAGUGGUGUU |
| | Exon 13 SA | CATTGTCTAG | CAUUGUCUAG |
| | (pos7) | GGATATAAAG | GGAUAUAAAG |
| | Exon 13 SA | ACATTGTCTA | ACAUUGUCUA |
| | (pos8) | GGGATATAAA | GGGAUAUAAA |
| | Exon 13 SA | GACATTGTCT | GACAUUGUCU |
| | (pos9) | AGGGATATAA | AGGGAUAUAA |
| | Exon 14 STOP | TGGCCAGGAC | UGGCCAGGAC |
| | (pos4) | ATTCCATCTT | AUUCCAucuu |
| | Exon 15 SA | CCATTCTAGG | CCAUUCUAGG |
| | (pos6) | AACAAAATAT | AACAAAAUAU |
| Cblb | Exon 1 STOP | GAGCTTCCAA | GAGCUUCCAA |
| | | GTCTTCTCCA | GUCUUCUCCA |
| | Exon 1 STOP | TCCCCGAAAA | UCCCCGAAAA |
| | (JMG44) | GGTCGAATTT | GGUCGAAUUU |
| | Exon 2 STOP | ATGAAGAACA | AUGAAGAACA |
| | | GTCACAGGAC | GUCACAGGAC |
| | Exon 3 SA | GATTTCGTCT | GAUUUCGUCU |
| | | GTAGGCACAA | GUAGGCACAA |
| | Exon 4 SD | TAAACTTACC | UAAACUUACC |
| | | TGAAACAGCC | UGAAACAGCC |
| | Exon 4 STOP | ATTCAGACAG | AUUCAGACAG |
| | | TGCCTTCATG | UGCCUUCAUG |
| | Exon 6 STOP | GTTGCACTCG | GUUGCACUCG |
| | | ATTGGGACAG | AUUGGGACAG |
| | Exon 6 STOP | TTATTTCAAG | UUAUUUCAAG |
| | | CCCTGATTGA | CCCUGAUUGA |
| | Exon 7 SD | TTACCTGTGT | UUACCUGUGU |
| | | AACTTTTATA | AACUUUUAUA |
| | Exon 8 SA | ATTGTTCCTG | AUUGUUCCUG |
| | (pos8) | GAATTTGGGG | GAAUUUGGGG |
| | Exon 8 SD | ATTATACCTG | AUUAUACCUG |
| | (JMG48) | CCATGCCGTA | CCAUGCCGUA |
| | Exon 8 SA | GTTCCTGGAA | GUUCCUGGAA |
| | (pos 5) | TTTGGGGAGG | UUUGGGGAGG |
| | (JMG46) | | |
| | Exon 8 STOP | CTGCCATGCC | CUGCCAUGCC |
| | (JMG47) | GTAAGGCAAG | GUAAGGCAAG |
| | Exon 10 SD | TCTACCTTTG | UCUACCUUUG |
| | (JMG49) | GTGAACCCGT | GUGAACCCGU |
| | Exon 11 SD | CTTACCTTAG | CUUACCUUAG |
| | (JMG50) | CTCCTTCTAA | CUCCUUCUAA |
| | Exon 11 STOP | GGGATGTCGA | GGGAUGUCGA |
| | | CTCCTAGGGG | CUCCUAGGGG |
| | Exon 11 STOP | CGAGGGCACC | CGAGGGCACC |
| | | ATGCTTCAAG | AUGCUUCAAG |
| | Exon 12 SD | AAACTCACTT | AAACUCACUU |
| | | TATGCTAGGG | UAUGCUAGGG |
| | Exon 12 SD | CTCACTTTAT | CUCACUUUAU |
| | (JMG51) | GCTAGGGAGG | GCUAGGGAGG |
| | Exon 16 SA | CTTCACCTGC | CUUCACCUGC |
| | (JMG52) | ATTTAAAGAA | AUUUAAAGAA |
| | Exon 4 STOP | CCACCAGATT | CCACCAGAUU |
| | (JMG45) | AGCTCTGGCC | AGCUCUGGCC |
| | Exon 10 SD | CTACCTTTGG | CUACCUUUGG |
| | (pos4) | TGAACCCGTT | UGAACCCGUU |
| BTLA | Exon 1 STOP | ATGTTCCAGA | AUGUUCCAGA |
| | (pos6) | TGTCCAGATA | UGUCCAGAUA |
| | Exon 1 STOP | TGTTCCAGAT | UGUUCCAGAU |
| | (pos5) | GTCCAGATAT | GUCCAGAUAU |
| | Exon 2 STOP | AGATAGACAA | AGAUAGACAA |
| | (pos8) | ACAAGTTGGA | ACAAGUUGGA |
| | Exon 2 STOP | AGCTTGCACC | AGCUUGCACC |
| | (pos9) | AAGTCACATG | AAGUCACAUG |
| | Exon 3 SD | ACCCACCTTG | ACCCACCUUG |
| | (pos6) | GTGCCTTCTC | GUGCCUUCUC |
| B2M | Exon 1 SD | ACTCACGCTG | ACUCACGCUG |
| (BE) | | GATAGCCTCC | GAUAGCCUCC |
| | Exon 2 SA | TGGAGTACCT | UGGAGUACCU |
| | (pos9) | GAGGAATATC | GAGGAAUAUC |
| | Exon 2 STOP | TTACCCCACT | UUACCCCACU |
| | (pos6) | TAACTATCTT | UAACUAUCUU |
| | Exon 3 SA | TCGATCTATG | UCGAUCUAUG |
| | | AAAAAGACAG | AAAAAGACAG |
| | Exon 2 STOP | TACCCCACTT | UACCCCACUU |
| | | AACTATCT | AACUAUCU |
| B2M | Exon 1 SD 1 | ACTCACGCTG | ACUCACGCUG |
| (ABE) | (pos 5) | GATAGCCTCC | GAUAGCCUCC |
| | Exon 2 SA | CTCAGGTACT | CUCAGGUACU |
| | (pos 4) | CCAAAGATTC | CCAAAGAUUC |
| | Exon 2 SD | CTTACCCCAC | CUUACCCCAC |
| | (pos 4) | TTAACTATCT | UUAACUAUCU |
| TET2 | Exon 1 STOP 1 | CATTTGCCAG | CAUUUGCCAG |
| | (pos 8) | ACAGAACCTC | ACAGAACCUC |
| | Exon 1 STOP 2 | AAACAAGACC | AAACAAGACC |
| | (pos 4) | AAAAGGCTAA | AAAAGGCUAA |
| | Exon 1 STOP 3 | GTAAGCCAAG | GUAAGCCAAG |
| | (pos 7) | AAAGAAATCC | AAAGAAAUCC |
| | Exon 1 STOP 4 | GCTTCAGATT | GCUUCAGAUU |
| | (pos 5) | CTGAATGAGC | CUGAAUGAGC |
| | Exon 1 STOP 5 | TTAAAACAAA | UUAAAACAAA |
| | (pos 7) | ATGAAATGAA | AUGAAAUGAA |
| | Exon 1 STOP 6 | GTTCCTCAGC | GUUCCUCAGC |
| | (pos 7) | TTCCTTCAGA | UUCCUUCAGA |
| | Exon 1 STOP 7 | CAAAGAGCAA | CAAAGAGCAA |
| | (pos 8) | GAGATTCTGA | GAGAUUCUGA |
| | Exon 1 STOP 8 | AAAGAGCAAG | AAAGAGCAAG |
| | (pos 7) | AGATTCTGAA | AGAUUCUGAA |
| | Exon 1 STOP 9 | ACACAGCACT | ACACAGCACU |
| | (pos 4) | ATCTGAAACC | AUCUGAAACC |
| | Exon 1 STOP 10 | CACCCAGAAA | CACCCAGAAA |
| | (pos | ACAACACAGC | ACAACACAGC |
| | 5) | | |
| | Exon 16 SA | CTTCACCTGC | CUUCACCUGC |
| | (JMG52) | ATTTAAAGAA | AUUUAAAGAA |
| | Exon 4 | CCACCAGATT | CCACCAGAUU |
| | STOP | AGCTCTGGCC | AGCUCUGGCC |
| | (JMG45) | | |
| | Exon 10 SD | CTACCTTTGG | CUACCUUUGG |
| | (pos4) | TGAACCCGTT | UGAACCCGUU |
| BTLA | Exon 1 | ATGTTCCAGA | AUGUUCCAGA |
| | STOP | TGTCCAGATA | UGUCCAGAUA |
| | (pos6) | | |
| | Exon 1 | TGTTCCAGAT | UGUUCCAGAU |
| | STOP | GTCCAGATAT | GUCCAGAUAU |
| | (pos5) | | |
| | Exon 2 | AGATAGACAA | AGAUAGACAA |
| | STOP | ACAAGTTGGA | ACAAGUUGGA |
| | (pos8) | | |
| | Exon 2 | AGCTTGCACC | AGCUUGCACC |
| | STOP | AAGTCACATG | AAGUCACAUG |
| | (pos9) | | |
| | Exon 3 SD | ACCCACCTTG | ACCCACCUUG |
| | (pos6) | GTGCCTTCTC | GUGCCUUCUC |
| B2M | Exon 1 SD | ACTCACGCTG | ACUCACGCUG |
| (BE) | | GATAGCCTCC | GAUAGCCUCC |
| | Exon 2 SA | TGGAGTACCT | UGGAGUACCU |
| | (pos9) | GAGGAATATC | GAGGAAUAUC |
| | Exon 2 | TTACCCCACT | UUACCCCACU |
| | STOP | TAACTATCTT | UAACUAUCUU |
| | (pos6) | | |
| | Exon 3 SA | TCGATCTATG | UCGAUCUAUG |
| | | AAAAAGACAG | AAAAAGACAG |
| | Exon 2 | TACCCCACTT | UACCCCACUU |
| | STOP | AACTATCT | AACUAUCU |
| B2M | Exon 1 SD 1 | ACTCACGCTG | ACUCACGCUG |
| (ABE) | (pos 5) | GATAGCCTCC | GAUAGCCUCC |
| | Exon 2 SA | CTCAGGTACT | CUCAGGUACU |
| | (pos 4) | CCAAAGATTC | CCAAAGAUUC |
| | Exon 2 SD | CTTACCCCAC | CUUACCCCAC |
| | (pos 4) | TTAACTATCT | UUAACUAUCU |
| TET2 | Exon 1 | CATTTGCCAG | CAUUUGCCAG |
| | STOP 1 | ACAGAACCTC | ACAGAACCUC |
| | (pos 8) | | |
| | Exon 1 | AAACAAGACC | AAACAAGACC |
| | STOP 2 | AAAAGGCTAA | AAAAGGCUAA |
| | (pos 4) | | |
| | Exon 1 | GTAAGCCAAG | GUAAGCCAAG |
| | STOP 3 | AAAGAAATCC | AAAGAAAUCC |
| | (pos 7) | | |
| | Exon 1 | GCTTCAGATT | GCUUCAGAUU |
| | STOP 4 | CTGAATGAGC | CUGAAUGAGC |
| | (pos 5) | | |
| | Exon 1 | TTAAAACAAA | UUAAAACAAA |
| | STOP 5 | ATGAAATGAA | AUGAAAUGAA |
| | (pos 7) | | |
| | Exon 1 | GTTCCTCAGC | GUUCCUCAGC |
| | STOP 6 | TTCCTTCAGA | UUCCUUCAGA |
| | (pos 7) | | |
| | Exon 1 | CAAAGAGCAA | CAAAGAGCAA |
| | STOP 7 | GAGATTCTGA | GAGAUUCUGA |
| | (pos 8) | | |
| | Exon 1 | AAAGAGCAAG | AAAGAGCAAG |
| | STOP 8 | AGATTCTGAA | AGAUUCUGAA |
| | (pos 7) | | |
| | Exon 1 | ACACAGCACT | ACACAGCACU |
| | STOP 9 | ATCTGAAACC | AUCUGAAACC |
| | (pos 4) | | |
| | Exon 1 | CACCCAGAAA | CACCCAGAAA |
| | STOP 10 | ACAACACAGC | ACAACACAGC |
| | (pos 5) | | |
| | Exon 1 | TACCAAGTTG | UACCAAGUUG |
| | STOP 11 | AAATGAATCA | AAAUGAAUCA |
| | (pos 4) | | |
| | Exon 1 | ATGAATCAAG | AUGAAUCAAG |
| | STOP 12 | GGCAGTCCCA | GGCAGUCCCA |
| | (pos 7) | | |
| | Exon 1 | AGGGCAGTCC | AGGGCAGUCC |
| | STOP 13 | CAAGGTACAG | CAAGGUACAG |
| | (pos 5) | | |
| | Exon 1 | GTTCCAAAAA | GUUCCAAAAA |
| | STOP 14 | CCCTCACACC | CCCUCACACC |
| | (pos 5) | | |
| | Exon 1 | GAAACAGCAC | GAAACAGCAC |
| | STOP 15 | TTGAATCAAC | UUGAAUCAAC |
| | (pos 5) | | |
| | Exon 1 | ATTACAAATA | AUUACAAAUA |
| | STOP 16 | AAGAATAAAG | AAGAAUAAAG |
| | (pos 5) | | |
| | Exon 1 | TAATGTCCAA | UAAUGUCCAA |
| | STOP 17 | ATGGGACTGG | AUGGGACUGG |
| | (pos 8) | | |
| | Exon 1 | CAAAGCAAGA | CAAAGCAAGA |
| | STOP 18 | TCTTCTTCAC | UCUUCUUCAC |
| | (pos 6) | | |
| | Exon 1 | ACAACAAGCT | ACAACAAGCU |
| | STOP 19 | TCAGTTCTAC | UCAGUUCUAC |
| | (pos 5) | | |
| | Exon 1 | CTGCGCAACT | CUGCGCAACU |
| | STOP 20 | TGCTCAGCAA | UGCUCAGCAA |
| | (pos 6) | | |
| | Exon 1 | CACTCAGACC | CACUCAGACC |
| | STOP 21 | CCTCCCCAGA | CCUCCCCAGA |
| | (pos 5) | | |
| | Exon 1 | TTTTTCCATG | UUUUUCCAUG |
| | STOP 22 | TTTTGTTTTC | UUUUGUUUUC |
| | (pos 6) | | |
| | Exon 1 SD | TTACCTACAC | UUACCUACAC |
| | (pos 4) | ATCTGCAAGA | AUCUGCAAGA |
| | Exon 3 SD | ACACTTACCC | ACACUUACCC |
| | (pos 8) | ACTTAGCAAT | ACUUAGCAAU |
| | Exon 7 | CATGCAGAAT | CAUGCAGAAU |
| | STOP | GGCAGCACAT | GGCAGCACAU |
| | (pos 5) | | |
| | Exon 8 | AAGCTCAGGA | AAGCUCAGGA |
| | STOP 1 | GGAGAAAAAA | GGAGAAAAAA |
| | (pos 6) | | |
| | Exon 8 | CGCAAGCCAG | CGCAAGCCAG |
| | STOP 2 | GCTAAACAGT | GCUAAACAGU |
| | (pos 8) | | |
| | Exon 9 | TTCTCCOCAG | UUCUCCCCAG |
| | STOP 1 | TCTCAGCCGA | UCUCAGCCGA |
| | (pos 8) | | |
| | Exon 9 | TGGTCAGGAA | UGGUCAGGAA |
| | STOP 2 | AAGCAGCCAT | AAGCAGCCAU |
| | (pos 5) | | |
| | Exon 9 | CTAGTCCAGG | CUAGUCCAGG |
| | STOP 3 | GTGTGGCTTC | GUGUGGCUUC |
| | (pos 7) | | |
| Spry1 | Exon 1 | CCCCAAAATC | CCCCAAAAUC |
| | STOP 1 | AACATGGCAG | AACAUGGCAG |
| | Exon 1 | TGTGATCCAG | UGUGAUCCAG |
| | STOP 2 | CAGCCTTCTT | CAGCCUUCUU |
| | Exon 1 | GACCAGATCA | GACCAGAUCA |
| | STOP 3 | AGGCCATAAG | AGGCCAUAAG |
| | Exon 1 | CAAGACAAGA | CAAGACAAGA |
| | STOP 4 | AAAGCATGAA | AAAGCAUGAA |
| | Exon 1 | CTGAACAGGG | CUGAACAGGG |
| | STOP 5 | ACTGTTAGGA | ACUGUUAGGA |
| Spry2 | Exon 1 | CCAGAGCTCA | CCAGAGCUCA |
| | STOP 1 | GAGTGGCAAC | GAGUGGCAAC |
| | Exon 1 | TTGCTGCAGA | UUGCUGCAGA |
| | STOP 2 | CGCCCCGTGA | CGCCCCGUGA |
| | Exon 1 | CTGCAGACGC | CUGCAGACGC |
| | STOP 3 | CCCGTGACGG | CCCGUGACGG |
| | Exon 1 | CGACAAGCAG | CGACAAGCAG |
| | STOP 4 | TGCCTTTGCT | UGCCUUUGCU |
| | Exon 1 | GCCCAGAACG | GCCCAGAACG |
| | STOP 5 | TGATTGACTA | UGAUUGACUA |
| | Exon 1 | TGTGCCAGGG | UGUGCCAGGG |
| | STOP 6 | GTGTTATGAC | GUGUUAUGAC |
| | Exon 1 | CAGATCCAGT | CAGAUCCAGU |
| | STOP 7 | CTGATGGCAG | CUGAUGGCAG |
| | Exon 1 | TGTACACGAT | UGUACACGAU |
| | STOP 8 | GGTCAGCCAT | GGUCAGCCAU |
| CIITA | Exon 1 SD | TTTTACCTTG | UUUUACCUUG |
| | (pos 6) | GGGCTCTGAC | GGGCUCUGAC |
| | Exon 1 | AGCCCCAAGG | AGCCCCAAGG |
| | STOP 1 | TAAAAAGGCC | UAAAAAGGCC |
| | (pos 6) | | |
| | Exon 1 | GAGCCCCAAG | GAGCCCCAAG |
| | STOP 2 | GTAAAAAGGC | GUAAAAAGGC |
| | (pos 7) | | |
| | Exon 2 | CAGCTCACAG | CAGCUCACAG |
| | STOP 1 | TGTGCCACCA | UGUGCCACCA |
| | (pos 8) | | |
| | Exon 2 | TATGACCAGA | UAUGACCAGA |
| | STOP 2 | TGGACCTGGC | UGGACCUGGC |
| | (pos 7) | | |
| | Exon 4 | ACTGGACCAG | ACUGGACCAG |
| | STOP 1 | TATGTCTTCC | UAUGUCUUCC |
| | (pos 8) | | |
| | Exon 4 | TGTCTTCCAG | UGUCUUCCAG |
| | STOP 2 | GACTCCCAGC | GACUCCCAGC |
| | (pos 8) | | |
| | Exon 7 | TTCAACCAGG | UUCAACCAGG |
| | STOP 1 | AGCCAGCCTC | AGCCAGCCUC |
| | (pos 7) | | |
| | Exon 7 | GACCAGATTC | GACCAGAUUC |
| | STOP 2 | CCAGTATGTT | CCAGUAUGUU |
| | (pos 4) | | |
| | Exon 7 SD | TAACATACTG | UAACAUACUG |
| | (pos 8) | GGAATCTGGT | GGAAUCUGGU |
| | Exon 8 SA | AAAGGCACTG | AAAGGCACUG |
| | (pos 8) | CAAGAGACAA | CAAGAGACAA |
| | Exon 8 | CTCTGGCAAA | CUCUGGCAAA |
| | STOP | TCTCTGAGGC | UCUCUGAGGC |
| | (pos 8) | | |
| | Exon 9 | AGCCAAGTAC | AGCCAAGUAC |
| | STOP 1 | CCCCTCCCAG | CCCCUCCCAG |
| | (pos 4) | | |
| | Exon 9 | ACCTCCCGAG | ACCUCCCGAG |
| | STOP 2 | CAAACATGAC | CAAACAUGAC |
| | (pos 7) | | |
| | Exon 9 SD | CCTTACCTGT | CCUUACCUGU |
| | (pos 6) | CATGTTTGCT | CAUGUUUGCU |
| | Exon 10 SA | TGCTCTGGAG | UGCUCUGGAG |
| | (pos 5) | ATGGAGAAGC | AUGGAGAAGC |
| | Exon 10 | CCCACCCAAT | CCCACCCAAU |
| | STOP 1 | GCCCGGCAGC | GCCCGGCAGC |
| | (pos 7) | | |
| | Exon 10 | AGGCCATTTT | AGGCCAUUUU |
| | STOP 2 | GGAAGCTTGT | GGAAGCUUGU |
| | (pos 4) | | |
| | Exon 11 SA | ACCGGCTCTG | ACCGGCUCUG |
| | (pos 8) | CAAAGGCCAG | CAAAGGCCAG |
| | Exon 11 | TGGTGCAGGC | UGGUGCAGGC |
| | STOP 1 | CAGGCTGGAG | CAGGCUGGAG |
| | (pos 6) | | |
| | Exon 11 | GAACGGCAGC | GAACGGCAGC |
| | STOP 3 | TGGCCCAAGG | UGGCCCAAGG |
| | (pos 7) | | |
| | Exon 11 | GGCCCAAGGA | GGCCCAAGGA |
| | STOP 4 | GGCCTGGCTG | GGCCUGGCUG |
| | (pos 5) | | |
| | Exon 11 | GACACGAGTG | GACACGAGUG |
| | STOP 5 | ATTGCTGTGC | AUUGCUGUGC |
| | (pos 5) | | |
| | Exon 11 | CTGGTCAGGG | CUGGUCAGGG |
| | STOP 5 | CAAGAGCTAT | CAAGAGCUAU |
| | (pos 6) | | |
| | Exon 11 | GGGCCCACAG | GGGCCCACAG |
| | STOP 5 | CCACTCGTGG | CCACUCGUGG |
| | (pos 8) | | |
| | Exon 11 | TTCCAGAAGA | UUCCAGAAGA |
| | STOP 6 | AGCTGCTCCG | AGCUGCUCCG |
| | (pos 4) | | |
| | Exon 11 | CCTGGTCCAG | CCUGGUCCAG |
| | STOP 7 | AGCCTGAGCA | AGCCUGAGCA |
| | (pos 8) | | |
| | Exon 11 | CAGACATCAA | CAGACAUCAA |
| | STOP 8 | AGTACCCTAC | AGUACCCUAC |
| | (pos 8) | | |
| | Exon 11 | ACATCAAAGT | ACAUCAAAGU |
| | STOP 9 | ACCCTACAGG | ACCCUACAGG |
| | (pos 5) | | |
| | Exon 11 | CGCCCAGGTC | CGCCCAGGUC |
| | STOP 10 | CTCACGTCTG | CUCACGUCUG |
| | (pos 4) | | |
| | Exon 11 | CTTAGTCCAA | CUUAGUCCAA |
| | STOP 11 | CACCCACCGC | CACCCACCGC |
| | (pos 8) | | |
| | Exon 11 | CCTCCTGCAA | CCUCCUGCAA |
| | STOP 12 | TGCTTCCTGG | UGCUUCCUGG |
| | (pos 8) | | |
| | Exon 11 | GAGCCAGCCA | GAGCCAGCCA |
| | STOP 13 | CAGGGCCCCC | CAGGGCCCCC |
| | (pos 8) | | |
| | Exon 11 | GGAAGCAGAA | GGAAGCAGAA |
| | STOP 14 | GGTGCTTGCG | GGUGCUUGCG |
| | (pos 6) | | |
| | Exon 11 | GGCTGCAGCC | GGCUGCAGCC |
| | STOP 15 | GGGGACACTG | GGGGACACUG |
| | (pos 6) | | |
| | Exon 11 | CTGCCAAATT | CUGCCAAAUU |
| | STOP 16 | CCAGCCTCCT | CCAGCCUCCU |
| | (pos 4) | | |
| | Exon 11 | GGCGGGCCAA | GGCGGGCCAA |
| | STOP 17 | GACTTCTCCC | GACUUCUCCC |
| | (pos 8) | | |
| | Exon 12 | AGACTCAGAG | AGACUCAGAG |
| | STOP 1 | GTGAGAGGAG | GUGAGAGGAG |
| | (pos 6) | | |
| | Exon 14 SA | AGCCTAGGAG | AGCCUAGGAG |
| | (pos 4) | GCAAAGAGCA | GCAAAGAGCA |
| | Exon 14 | CCCCCAGGCT | CCCCCAGGCU |
| | STOP 1 | TTCCCCAAAC | UUCCCCAAAC |
| | (pos 5) | | |
| | Exon 14 SD | TCACTCCAGA | UCACUCCAGA |
| | (pos 4) | TGCTGCAGGG | UGCUGCAGGG |
| | Exon 15 SA | AGGCTGCAGG | AGGCUGCAGG |
| | (pos 4) | TGGAATCAGA | UGGAAUCAGA |
| | Exon 15 | CTTCCCCCAG | CUUCCCCCAG |
| | STOP 1 | CTGAAGTCCT | CUGAAGUCCU |
| | (pos 8) | | |
| | Exon 15 SD | CACTCACTTG | CACUCACUUG |
| | (pos 7) | AGGGTTTCCA | AGGGUUUCCA |
| | Exon 16 SA | CAGACTGCGG | CAGACUGCGG |
| | (pos 5) | GGACACAGTG | GGACACAGUG |
| | Exon 16 SD 1 | CCACTCACCT | CCACUCACCU |
| | (pos 8) | TAGCCTGAGC | UAGCCUGAGC |
| | Exon 16 SD 2 | CACTCACCTT | CACUCACCUU |
| | (pos 7) | AGCCTGAGCA | AGCCUGAGCA |
| | Exon 17 SA | GTACAAGCTG | GUACAAGCUG |
| | (pos 8) | TCGGAAACAG | UCGGAAACAG |
| | Exon 17 SD 1 | ACACTCACTC | ACACUCACUC |
| | (pos 8) | CATCACCCGG | CAUCACCCGG |
| | Exon 17 SD 2 | CACTCACTCC | CACUCACUCC |
| | (pos 7) | ATCACCCGGA | AUCACCCGGA |
| | Exon 18 | CGTCCAGTAC | CGUCCAGUAC |
| | STOP | AACAAGTTCA | AACAAGUUCA |
| | (pos 5) | | |
| | Exon 19 SA 1 | CCACATCCTG | CCACAUCCUG |
| | (pos 8) | CAAGGGGGGA | CAAGGGGGGA |
| | Exon 19 SA 2 | CACATCCTGC | CACAUCCUGC |
| | (pos 7) | AAGGGGGGAT | AAGGGGGGAU |
| | Exon 19 | TGGGCGTCCA | UGGGCGUCCA |
| | STOP 1 | CATCCTGCAA | CAUCCUGCAA |
| | (pos 8) | | |
| | Exon 19 | GGGCGTCCAC | GGGCGUCCAC |
| | STOP 2 | ATCCTGCAAG | AUCCUGCAAG |
| | (pos 9) | | |
| | Exon 19 | GGCGTCCACA | GGCGUCCACA |
| | STOP 3 | TCCTGCAAGG | UCCUGCAAGG |
| | (pos 6) | | |
| | Exon 19 | GCGTCCACAT | GCGUCCACAU |
| | STOP 4 | CCTGCAAGGG | CCUGCAAGGG |
| | (pos 5) | | |
| CD7 | Exon 1 | GCCCAAGGTA | GCCCAAGGUA |
| | STOP | AGAGCTTCCC | AGAGCUUCCC |
| | (pos 4) | | |
| | Exon 1 SD 1 | GCTCTTACCT | GCUCUUACCU |
| | (pos 8) | TGGGCAGCCA | UGGGCAGCCA |
| | Exon 1 SD 2 | AGCTCTTACC | AGCUCUUACC |
| | (pos 9) | TTGGGCAGCC | UUGGGCAGCC |
| | Exon 2 SA 1 | TGCACCTCTG | UGCACCUCUG |
| | (pos 8) | GGGAGGACCT | GGGAGGACCU |
| | Exon 2 SA 2 | CTGCACCTCT | CUGCACCUCU |
| | (pos 9) | GGGGAGGACC | GGGGAGGACC |
| | Exon 2 | CGCCTGCAGC | CGCCUGCAGC |
| | STOP 1 | TGTCGGACAC | UGUCGGACAC |
| | (pos 7) | | |
| | Exon 2 | CACCTGCCAG | CACCUGCCAG |
| | STOP 2 | GCCATCACGG | GCCAUCACGG |
| | (pos 8) | | |
| | Exon 2 SD 1 | CCCTACCTGT | CCCUACCUGU |
| | (pos 6) | CACCAGGACC | CACCAGGACC |
| | Exon 2 SD 2 | CCTACCTGTC | CCUACCUGUC |
| | (pos 5) | ACCAGGACCA | ACCAGGACCA |
| | Exon 3 SA | CCTCTGAGAA | CCUCUGAGAA |
| | (pos 4) | GGAAAAAAGA | GGAAAAAAGA |
| | Exon 3 | CAGAGGAACA | CAGAGGAACA |
| | STOP 1 | GTCCCAAGGA | GUCCCAAGGA |
| | (pos9) | | |
| CD33 | Exon 1 SD 1 | CACTCACCTG | CACUCACCUG |
| | (pos 7) | CCCACAGCAG | CCCACAGCAG |
| | Exon 1 SD 2 | CCACTCACCT | CCACUCACCU |
| | (pos 8) | GCCCACAGCA | GCCCACAGCA |
| | Exon 1 SD | GCCACTCACC | GCCACUCACC |
| | (pos 9) | TGCCCACAGC | UGCCCACAGC |
| | Exon 2 SA 1 | AGGGCCCCTG | AGGGCCCCUG |
| | (pos 8) | TGGGGAAACG | UGGGGAAACG |
| | Exon 2 SA 2 | GGGCCCCTGT | GGGCCCCUGU |
| | (pos 7) | GGGGAAACGA | GGGGAAACGA |
| | Exon 2 | GCAAGTGCAG | GCAAGUGCAG |
| | STOP 1 | GAGTCAGTGA | GAGUCAGUGA |
| | (pos 8) | | |
| | Exon 2 | CGGAACCAGT | CGGAACCAGU |
| | STOP 2 | AACCATGAAC | AACCAUGAAC |
| | (pos 6) | | |
| | Exon 2 | GGAACCAGTA | GGAACCAGUA |
| | STOP 3 | ACCATGAACT | ACCAUGAACU |
| | (pos 5) | | |
| | Exon 2 | GAACCAGTAA | GAACCAGUAA |
| | STOP 4 | CCATGAACTG | CCAUGAACUG |
| | (pos 4) | | |
| | Exon 2 | GCTAGATCAA | GCUAGAUCAA |
| | STOP 5 | GAAGTACAGG | GAAGUACAGG |
| | (pos 8) | | |
| | Exon 2 | AGAAGTACAG | AGAAGUACAG |
| | STOP 6 | GAGGAGACTC | GAGGAGACUC |
| | (pos 8) | | |
| | Exon 3 SA 1 | CAAGTCTAGT | CAAGUCUAGU |
| | (pos 6) | GAGGAGAAAG | GAGGAGAAAG |
| | Exon 3 SA 2 | AAGTCTAGTG | AAGUCUAGUG |
| | (pos 5) | AGGAGAAAGA | AGGAGAAAGA |
| | Exon 3 SA 3 | AGTCTAGTGA | AGUCUAGUGA |
| | (pos 4) | GGAGAAAGAG | GGAGAAAGAG |
| | Exon 3 | ACAGGCCCAG | ACAGGCCCAG |
| | STOP 1 | GACACAGAGC | GACACAGAGC |
| | (pos 7) | | |
| | Exon 3 | ACCTGTCAGG | ACCUGUCAGG |
| | STOP 2 | TGAAGTTCGC | UGAAGUUCGC |
| | (pos 7) | | |
| | Exon 3 SD 1 | ACTTACAGGT | ACUUACAGGU |
| | (pos 6) | GACGTTGAGC | GACGUUGAGC |
| | Exon 4 SA 1 | AACATCTAGG | AACAUCUAGG |
| | (pos 6) | AGAGGAAGAG | AGAGGAAGAG |
| | Exon 4 | GTTCCACAGA | GUUCCACAGA |
| | STOP 1 | ACCCAACAAC | ACCCAACAAC |
| | (pos 7) | | |
| | Exon 4 SD 1 | TTCCTACCTG | UUCCUACCUG |
| | (pos 7) | AGCCATCTCC | AGCCAUCUCC |
| | Exon 5 SD | ATGCTCACAT | AUGCUCACAU |
| | (pos 8) | GAAGAAGATG | GAAGAAGAUG |
| | Exon 5 | GGGAAACAAG | GGGAAACAAG |
| | STOP 1 | AGACCAGAGC | AGACCAGAGC |
| | (pos 7) | | |
| | Exon 6 SA 1 | TCACTCTGAT | UCACUCUGAU |
| | (pos 6) | GGGAGACACC | GGGAGACACC |
| | Exon 6 SA 2 | CACTCTGATG | CACUCUGAUG |
| | (pos 5) | GGAGACACCA | GGAGACACCA |
| | Exon 6 SA 1 | TTTCTTATGG | UUUCUUAUGG |
| | (pos 4) | AGAGGAAAGA | AGAGGAAAGA |
| CD52 | Exon 1 | GTACAGGTAA | GUACAGGUAA |
| | STOP | GAGCAACGCC | GAGCAACGCC |
| | (pos 4) | | |
| | Exon 1 SD | CTCTTACCTG | CUCUUACCUG |
| | (pos7) | TACCATAACC | UACCAUAACC |
| | Exon 1 SD | TTACCTGTAC | UUACCUGUAC |
| | (pos 4) | CATAACCAGG | CAUAACCAGG |
| | Exon 2 SA | TGTATCTGTA | UGUAUCUGUA |
| | (pos 6) | GGAGGAGAAG | GGAGGAGAAG |
| | Exon 2 SA | GTATCTGTAG | GUAUCUGUAG |
| | (pos 5) | GAGGAGAAGT | GAGGAGAAGU |
| | Exon 2 | CAGATACAAA | CAGAUACAAA |
| | STOP | CTGGACTCTC | CUGGACUCUC |
| | (pos 7) | | |
| CD123 | Exon 1 SD | TCTTACCTTC | UCUUACCUUC |
| | (pos 6) | CTTCGTTTGC | CUUCGUUUGC |
| | Exon 2 SA 1 | TTTGGATCTA | UUUGGAUCUA |
| | (pos 8) | AAACGGTGAC | AAACGGUGAC |
| | Exon 2 SA 2 | GATCTAAAAC | GAUCUAAAAC |
| | (pos 4) | GGTGACAGGT | GGUGACAGGU |
| | Exon 2 | AAAGGCTCAG | AAAGGCUCAG |
| | STOP 1 | CAGTTGACCT | CAGUUGACCU |
| | (pos 8) | | |
| | Exon 2 SD | ATTTACCGGC | AUUUACCGGC |
| | (pos 6) | ATAGAATAGT | AUAGAAUAGU |
| | Exon 3 SA | TCACTGCCTA | UCACUGCCUA |
| | (pos 8) | AGAGAGACAT | AGAGAGACAU |
| | Exon 3 | AGGATCCACG | AGGAUCCACG |
| | STOP 1 | TGGAGAATGG | UGGAGAAUGG |
| | (pos 6) | | |
| | Exon 3 | GGATCCACGT | GGAUCCACGU |
| | STOP 2 | GGAGAATGGT | GGAGAAUGGU |
| | (pos 5) | | |
| | Exon 3 SD | TCTCACTGTT | UCUCACUGUU |
| | (pos 6) | CTCAGGGAAG | CUCAGGGAAG |
| | Exon 4 | CCTGCCCAAG | CCUGCCCAAG |
| | STOP 1 | GCTTCCCACC | GCUUCCCACC |
| | (pos 6) | | |
| | Exon 4 | CTGCCCAAGG | CUGCCCAAGG |
| | STOP 2 | CTTCCCACCT | CUUCCCACCU |
| | (pos 5) | | |
| | Exon 5 SA 1 | GCCTGCTGCG | GCCUGCUGCG |
| | (pos 6) | GTAAGCGGTA | GUAAGCGGUA |
| | Exon 5 | GATGCTCAGG | GAUGCUCAGG |
| | STOP 1 | GAACACGTAT | GAACACGUAU |
| | (pos 7) | | |
| | Exon 5 | TTCTCAAAGT | UUCUCAAAGU |
| | STOP 2 | TCCCACATCC | UCCCACAUCC |
| | (pos 5) | | |
| | Exon 5 | TCACAGATTG | UCACAGAUUG |
| | STOP 3 | GTGAGTAGCC | GUGAGUAGCC |
| | (pos 4) | | |
| | Exon 7 SD | CTCACCTGTT | CUCACCUGUU |
| | (pos 5) | CTGTGATTAC | CUGUGAUUAC |
| | Exon 8 | TCCTTCCAGC | UCCUUCCAGC |
| | STOP 1 | TACTCAATCC | UACUCAAUCC |
| | (pos 7) | | |
| | Exon 8 | CACAGTACAA | CACAGUACAA |
| | STOP 2 | ATAAGAGCCC | AUAAGAGCCC |
| | (pos 8) | | |
| | Exon 8 | CCCCCCAGCG | CCCCCCAGCG |
| | STOP 3 | CTTCGGTGAG | CUUCGGUGAG |
| | (pos 6) | | |
| | Exon 8 | CCCCCAGCGC | CCCCCAGCGC |
| | STOP 4 | TTCGGTGAGT | UUCGGUGAGU |
| | (pos 5) | | |
| | Exon 8 SD | CCACTCACCG | CCACUCACCG |
| | (pos 8) | AAGCGCTGGG | AAGCGCUGGG |
| | Exon 10 SA | TACCTCGGAG | UACCUCGGAG |
| | (pos 4) | GAAAGAGAAA | GAAAGAGAAA |
| | Exon 10 | CAGCTTCCAA | CAGCUUCCAA |
| | STOP | AACGACAAGC | AACGACAAGC |
| | (pos 8) | | |
| | Exon 10 SD | AACATACCAG | AACAUACCAG |
| | (pos 7) | CTTGTCGTTT | CUUGUCGUUU |
| | Exon 11 SA 1 | AGACCACCTG | AGACCACCUG |
| | (pos 8) | CAGAGAGGAG | CAGAGACGAG |
| | Exon 11 SA 2 | CCACCTGCAG | CCACCUGCAG |
| | (pos 5) | AGACGAGAGG | AGACGAGAGG |
| TRBC1 | Exon 1 | CCACACCCAA | CCACACCCAA |
| | STOP 1 | AAGGCCACAC | AAGGCCACAC |
| | (pos 8) | | |
| | Exon 1 | CCCACCAGCT | CCCACCAGCU |
| | STOP 2 | CAGCTCCACG | CAGCUCCACG |
| | (pos 5) | | |
| | Exon 1 | CGCTGTCAAG | CGCUGUCAAG |
| | STOP 3 | TCCAGTTCTA | UCCAGUUCUA |
| | (pos 7) | | |
| | Exon 1 | GCTGTCAAGT | GCUGUCAAGU |
| | STOP 4 | CCAGTTCTAC | CCAGUUCUAC |
| | (pos 6) | | |
| | Exon 1 | CACCCAGATC | CACCCAGAUC |
| | STOP 5 | GTCAGCGCCG | GUCAGCGCCG |
| | (pos 5) | | |
| | Exon 1 SD | CCACTCACCT | CCACUCACCU |
| | (pos 8) | GCTCTACCCC | GCUCUACCCC |
| | Exon 2 SA | CCACAGTCTG | CCACAGUCUG |
| | (pos 8) | AAAGAAAGCA | AAAGAAAGCA |
| | Exon 3 SA | GACACTGTTG | GACACUGUUG |
| | (pos 5) | GCACGGAGGA | GCACGGAGGA |
| | Exon 3 SD | TTACCATGGC | UUACCAUGGC |
| | (pos 4) | CATCAACACA | CAUCAACACA |
| TRBC2 | Exon 1 | CCACACCCAA | CCACACCCAA |
| | STOP 1 | AAGGCCACAC | AAGGCCACAC |
| | (pos 8) | | |
| | Exon 1 | CCCACCAGCT | CCCACCAGCU |
| | STOP 2 | CAGCTCCACG | CAGCUCCACG |
| | (pos 5) | | |
| | Exon 1 | CGCTGTCAAG | CGCUGUCAAG |
| | STOP 3 | TCCAGTTCTA | UCCAGUUCUA |
| | (pos 7) | | |
| | Exon 1 | GCTGTCAAGT | GCUGUCAAGU |
| | STOP 4 | CCAGTTCTAC | CCAGUUCUAC |
| | (pos 6) | | |
| | Exon 1 | CACCCAGATC | CACCCAGAUC |
| | STOP 5 | GTCAGCGCCG | GUCAGCGCCG |
| | (pos 5) | | |
| | Exon 2 SA | CCACAGTCTG | CCACAGUCUG |
| | (pos 8) | AAAGAAAACA | AAAGAAAACA |
| | Exon 2 SA | CACAGTCTGA | CACAGUCUGA |
| | (pos 7) | AAGAAAACAG | AAGAAAACAG |
| | Exon 3 SD | TTACCATGGC | UUACCAUGGC |
| | (pos 4) | CATCAGCACG | CAUCAGCACG |
| | Exon 1 SD | CCACTCACCT | CCACUCACCU |
| | (pos 8) | GCTCTACCCC | GCUCUACCCC |
| CISH | Exon 1 | TCTGCGTTCA | UCUGCGUUCA |
| | STOP | GGGGTAAGCG | GGGGUAAGCG |
| | Exon 1 SD | GCGCTTACCC | GCGCUUACCC |
| | | CTGAACGCAG | CUGAACGCAG |
| | Exon 2 | GACTGGGCAG | GACUGGGCAG |
| | STOP 2 | CGGCCCCTGT | CGGCCCCUGU |
| | Exon 2 | GGACTGGGCA | GGACUGGGCA |
| | STOP 1 | GCGGCCCCTG | GCGGCCCCUG |
| | Exon 2 | GTCATGCAGC | GUCAUGCAGC |
| | STOP 3 | CCTTGCCTGC | CCUUGCCUGC |
| | Exon 2 | TCATGCAGCC | UCAUGCAGCC |
| | STOP 4 | CTTGCCTGCT | CUUGCCUGCU |
| | Exon 2 | CATGCAGCCC | CAUGCAGCCC |
| | STOP 5 | TTGCCTGCTG | UUGCCUGCUG |
| | Exon 2 SD 1 | CTCACCAGAT | CUCACCAGAU |
| | | TCCCGAAGGT | UCCCGAAGGU |
| | Exon 2 SD 2 | CAGACTCACC | CAGACUCACC |
| | | AGATTCCCGA | AGAUUCCCGA |
| | Exon 3 SA 1 | AGCCTAGGCA | AGCCUAGGCA |
| | (pos 4) | AGTGCAGAGG | AGUGCAGAGG |
| | Exon 3 SA 2 | CAGCCTAGGC | CAGCCUAGGC |
| | (pos 5) | AAGTGCAGAG | AAGUGCAGAG |
| | Exon 3 SA 3 | ACCAGCCTAG | ACCAGCCUAG |
| | (pos 7) | GCAAGTGCAG | GCAAGUGCAG |
| | Exon 3 | TGGAACCCCA | UGGAACCCCA |
| | STOP 1 | ATACCAGCCT | AUACCAGCCU |
| | (pos 8) | | |
| | Exon 3 | CACCTGCAGA | CACCUGCAGA |
| | STOP 2 | AGATGCCAGA | AGAUGCCAGA |
| | (pos 7) | | |
| ACAT1 | Exon 1 SD 1 | CGCTCACCTG | CGCUCACCUG |
| | (pos 7) | CACCAGCCTC | CACCAGCCUC |
| | Exon 3 SA | CTTCCTGGCA | CUUCCUGGCA |
| | (pos 5) | AGACACAAGA | AGACACAAGA |
| | Exon 3 | AATTCAGGGA | AAUUCAGGGA |
| | STOP | GCCATTGAAA | GCCAUUGAAA |
| | (pos 5) | | |
| | Exon 3 SD | CTACTGACCT | CUACUGACCU |
| | (pos 8) | GCCTTTTCAA | GCCUUUUCAA |
| | Exon 5 | GCCTCTCAAA | GCCUCUCAAA |
| | STOP | GTCTTATGTG | GUCUUAUGUG |
| | (pos 7) | | |
| | Exon 7 | TTCCCATGCT | UUCCCAUGCU |
| | STOP | GCTTTACTTC | GCUUUACUUC |
| | (pos 4) | | |
| | Exon 8 | TTTAGGTCAA | UUUAGGUCAA |
| | STOP | CCAGATGTAG | CCAGAUGUAG |
| | (pos 8) | | |
| | Exon 9 SA | TGTGCCTGAA | UGUGCCUGAA |
| | (pos 9) | AGCAAAAATG | AGCAAAAAUG |
| | Exon 9 SD | TTACCTACTA | UUACCUACUA |
| | (pos 4) | TTCTTGCCAG | UUCUUGCCAG |
| | Exon 10 SA | AAATGCTGTT | AAAUGCUGUU |
| | (pos 6) | TAAAAAAAGG | UAAAAAAAGG |
| | Exon 11 | CCCCAAAAAG | CCCCAAAAAG |
| | STOP | TGAATATCAA | UGAAUAUCAA |
| | (pos 4) | | |
| Cyp11a | Exon 1 | GTCCAGAATT | GUCCAGAAUU |
| 1 | STOP 1 | TCCAGAAGTA | UCCAGAAGUA |
| | (pos 4) | | |
| | Exon 2 SA 1 | TCCCTGGAGG | UCCCUGGAGG |
| | (pos 4) | GGTGGGGGAG | GGUGGGGGAG |
| | Exon 2 SD 1 | TCACTTCAAC | UCACUUCAAC |
| | (pos 4) | AGGACTCCTA | AGGACUCCUA |
| | Exon 3 SD 1 | CCTTACACTC | CCUUACACUC |
| | (pos 6) | AAAGGCAAAG | AAAGGCAAAG |
| | Exon 4 SA | ATGGCTGCAG | AUGGCUGCAG |
| | (pos 5) | GGAGAGGAAG | GGAGAGGAAG |
| | Exon 4 | GGAGCGCCAG | GGAGCGCCAG |
| | STOP 1 | GGGATGCTGG | GGGAUGCUGG |
| | (pos 8) | | |
| | Exon 4 | TCACGTCCCA | UCACGUCCCA |
| | STOP 2 | TGCAGCCACA | UGCAGCCACA |
| | (pos 8) | | |
| | Exon 6 SA | TGGACGTCTG | UGGACGUCUG |
| | (pos 8) | GTGGGGAGTA | GUGGGGAGUA |
| | Exon 8 | ACTCACATTG | ACUCACAUUG |
| | STOP l | ATGAGGAAGA | AUGAGGAAGA |
| | (pos 6) | | |
| | Exon 9 SA | CAGCATCTGA | CAGCAUCUGA |
| | (pos 7) | GAAAGGCAGA | GAAAGGCAGA |
| | Exon 9 | AATCCAACAC | AAUCCAACAC |
| | STOP 1 | CTCAGCGATG | CUCAGCGAUG |
| | (pos 5) | | |
| | Exon 9 | ATCCAACACC | AUCCAACACC |
| | STOP 2 | TCAGCGATGT | UCAGCGAUGU |
| | (pos 4) | | |
| GATA3 | Exon 1 | CGCGGCGCAG | CGCGGCGCAG |
| | STOP 1 | TACCCGCTGC | UACCCGCUGC |
| | (pos 8) | | |
| | Exon 1 SD 1 | CACTCACCGT | CACUCACCGU |
| | (pos 7) | GGTGGGTCGG | GGUGGGUCGG |
| | Exon 1 SD 2 | ACTCACCGTG | ACUCACCGUG |
| | (pos 6) | GTGGGTCGGA | GUGGGUCGGA |
| | Exon 2 SA 1 | TGGCTCCCTG | UGGCUCCCUG |
| | (pos 8) | TGGGGCAACG | UGGGGCAACG |
| | Exon 2 | GATTCCAGGG | GAUUCCAGGG |
| | STOP 2 | GGAGGCGGTG | GGAGGCGGUG |
| | (pos 5) | | |
| | Exon 2 SD 1 | GCTCCTACCT | GCUCCUACCU |
| | (pos 8) | GTGCTGGACC | GUGCUGGACC |
| | Exon 3 | TCGCCGCCAC | UCGCCGCCAC |
| | STOP 1 | AGTGGGGTCG | AGUGGGGUCG |
| | (pos 7) | | |
| | Exon 4 SA | CAGACTGAGA | CAGACUGAGA |
| | (pos 5) | GTGGGGAGAG | GUGGGGAGAG |
| | Exon 4 | CCTCCTCCAG | CCUCCUCCAG |
| | STOP 1 | AGTGTGGTTG | AGUGUGGUUG |
| | (pos 7) | | |
| NR4A1 | Exon 1 | AGCCATCCCA | AGCCAUCCCA |
| | STOP 1 | GGGAGAGAGC | GGGAGAGAGC |
| | (pos 8) | | |
| | Exon 1 | GCCATCCCAG | GCCAUCCCAG |
| | STOP 2 | GGAGAGAGCT | GGAGAGAGCU |
| | (pos 7) | | |
| | Exon 1 | CCATCCCAGG | CCAUCCCAGG |
| | STOP 3 | GAGAGAGCTG | GAGAGAGCUG |
| | (pos 6) | | |
| | Exon 1 | CTCACAGGCC | CUCACAGGCC |
| | STOP 4 | ACCCACCAGC | ACCCACCAGC |
| | (pos 5) | | |
| | Exon 2 | CCGCTTCCAG | CCGCUUCCAG |
| | STOP 1 | AAGTGCCTGG | AAGUGCCUGG |
| | (pos 8) | | |
| | Exon 2 | CTTCCAGAAG | CUUCCAGAAG |
| | STOP 2 | TGCCTGGCGG | UGCCUGGCGG |
| | (pos 5) | | |
| | Exon 3 SA 1 | ACAACTGCAA | ACAACUGCAA |
| | (pos 5) | AGGAATGGGT | AGGAAUGGGU |
| | Exon 3 SA 2 | CAACTGCAAA | CAACUGCAAA |
| | (pos 4) | GGAATGGGTA | GGAAUGGGUA |
| | Exon 4 SA | GAACTAGGAA | GAACUAGGAA |
| | (pos 4) | GACGGTCCAG | GACGGUCCAG |
| | Exon 4 | GGCTGACCAG | GGCUGACCAG |
| | STOP 1 | GACCTGTTGC | GACCUGUUGC |
| | (pos 8) | | |
| | Exon 4 SD I | CTCACCTGTA | CUCACCUGUA |
| | (pos 5) | CGCCAGGCGG | CGCCAGGCGG |
| | Exon 4 SD 2 | GCTCTCACCT | GCUCUCACCU |
| | (pos 8) | GTACGCCAGG | GUACGCCAGG |
| | Exon 5 SA | CTTAGACCTG | CUUAGACCUG |
| | (pos 8) | GCAGGCAGAT | GCAGGCAGAU |
| | Exon 5 | CAATCCAGTC | CAAUCCAGUC |
| | STOP 1 | CCCGAAGCCA | CCCGAAGCCA |
| | (pos 5) | | |
| | Exon 5 | AATCCAGTCC | AAUCCAGUCC |
| | STOP 2 | CCGAAGCCAC | CCGAAGCCAC |
| | (pos 4) | | |
| | Exon 5 SD 1 | ACTCACCGGT | ACUCACCGGU |
| | (pos 6) | GATGAGGACA | GAUGAGGACA |
| | Exon 5 SD 2 | CTCACCGGTG | CUCACCGGUG |
| | (pos 5) | ATGAGGACAA | AUGAGGACAA |
| | Exon 6 SA | CCGGTCTGCG | CCGGUCUGCG |
| | (pos 6) | GGAAGGGTAC | GGAAGGGUAC |
| | Exon 6 | TGGGCTGCAG | UGGGCUGCAG |
| | STOP 1 | GAGCCGCGGC | GAGCCGCGGC |
| | (pos 8) | | |
| NR4A2 | Exon 1 | TTGTACCAAA | UUGUACCAAA |
| | STOP 1 | TGCCCCTGTC | UGCCCCUGUC |
| | (pos 7) | | |
| | Exon 1 | CGGACAGCAG | CGGACAGCAG |
| | STOP 2 | TCCTCCATTA | UCCUCCAUUA |
| | (pos 8) | | |
| | Exon 1 | AGGTGCAGCA | AGGUGCAGCA |
| | STOP 3 | CAGCCCCATG | CAGCCCCAUG |
| | (pos 6) | | |
| | Exon 1 | GGTGCAGCAC | GGUGCAGCAC |
| | STOP 4 | AGCCCCATGT | AGCCCCAUGU |
| | (pos 5) | | |
| | Exon 1 | AGTTGCCAGA | AGUUGCCAGA |
| | STOP 5 | TGCGCTTCGA | UGCGCUUCGA |
| | (pos 7) | | |
| | Exon 1 | GTTGCCAGAT | GUUGCCAGAU |
| | STOP 6 | GCGCTTCGAC | GCGCUUCGAC |
| | (pos 6) | | |
| | Exon 1 | GTCTCAGCTG | GUCUCAGCUG |
| | STOP 7 | CTCGACACGC | CUCGACACGC |
| | (pos 5) | | |
| | Exon 3 SD | TTCTTACCCT | UUCUUACCCU |
| | (pos 7) | GGAATAGTCC | GGAAUAGUCC |
| | Exon 4 SD | ATTACCTGTA | AUUACCUGUA |
| | (pos 5) | TGCTAATCGA | UGCUAAUCGA |
| | Exon 5 | TTGCAATGCG | UUGCAAUGCG |
| | STOP 1 | TTCGTGGCTT | UUCGUGGCUU |
| | (pos 4) | | |
| | Exon 5 SD | ACTGACCTGT | ACUGACCUGU |
| | (pos 6) | GACCATAGCC | GACCAUAGCC |
| NR4A3 | Exon 2 SA | TATCTGCAGG | UAUCUGCAGG |
| | (pos 4) | GACAGAGAAA | GACAGAGAAA |
| | Exon 2 | TGCGGCGCAG | UGCGGCGCAG |
| | STOP 1 | ACATACAGCT | ACAUACAGCU |
| | (pos 8) | | |
| | Exon 2 | CCCCGCAGGC | CCCCGCAGGC |
| | STOP 2 | GGGGGCGTTA | GGGGGCGUUA |
| | (pos 6) | | |
| | Exon 3 | TTTCAGAAGT | UUUCAGAAGU |
| | STOP 1 | GTCTCAGTGT | GUCUCAGUGU |
| | (pos 4) | | |
| | Exon 5 SD | ATTACCTGAT | AUUACCUGAU |
| | (pos 5) | GGAAAGTCTG | GGAAAGUCUG |
| | Exon 6 | CTTCAGTGCC | CUUCAGUGCC |
| | STOP 1 | TTCGTGGATT | UUCGUGGAUU |
| | (pos 4) | | |
| | Exon 7 SA | TTTCTGCAGA | UUUCUGCAGA |
| | (pos 4) | GGGATAGAGA | GGGAUAGAGA |
| | Exon 7 | AGACCACCAG | AGACCACCAG |
| | STOP 1 | AGTAAGGGAC | AGUAAGGGAC |
| | (pos 8) | | |
| MCJ | Exon 1 | ACTTGCAGCC | ACUUGCAGCC |
| | STOP | CTCGGCCAAA | CUCGGCCAAA |
| | (pos 6) | | |
| FAS | Exon 1 SD | AGGGCTCACC | AGGGCUCACC |
| | (pos 9) | AGAGGTAGGA | AGAGGUAGGA |
| | Exon 3 SA | TTCACCTGCC | UUCACCUGCC |
| | (pos 6) | CAAGGAAAAA | CAAGGAAAAA |
| | Exon 4 SA | CTAAGCCTAG | CUAAGCCUAG |
| | (pos 7) | AAAATCAGTT | AAAAUCAGUU |
| | Exon 5 SA | ACATCTAGAA | ACAUCUAGAA |
| | (pos 5) | AAAAAAATAC | AAAAAAAUAC |
| | Exon 5 SD | ATTACCTTCC | AUUACCUUCC |
| | (pos 5) | TCTTTGCACT | UCUUUGCACU |
| | Exon 6 SA | GATCCTGTAG | GAUCCUGUAG |
| | (pos 5) | GTTGGAACAT | GUUGGAACAU |
| | Exon 6 | AAGCCACCCC | AAGCCACCCC |
| | STOP 1 | AAGTTAGATC | AAGUUAGAUC |
| | (pos 4) | | |
| | Exon 6 SD | AACTTACCCC | AACUUACCCC |
| | (pos 7) | AAACAATTAG | AAACAAUUAG |
| | Exon 7 SD | ATACCTACAG | AUACCUACAG |
| | (pos 8) | GATTTAAAGT | GAUUUAAAGU |
| | Exon 8 SA | GTTTCCTAGA | GUUUCCUAGA |
| | (pos 8) | AAGCAAAAAA | AAGCAAAAAA |
| | Exon 9 | AAGTTCAACT | AAGUUCAACU |
| | STOP 1 | GCTTCGTAAT | GCUUCGUAAU |
| | (pos 6) | | |
| | Exon 9 | AATTCAGACT | AAUUCAGACU |
| | STOP | ATCATCCTCA | AUCAUCCUCA |
| | (pos 5) | | |
| SELPG/ | Exon1 | GCTTGCAGCT | GCUUGCAGCU |
| PSGL1 | STOP 1 | GTGGGACACC | GUGGGACACC |
| | (pos 6) | | |
| | Exon1 | GACCACTCAA | GACCACUCAA |
| | STOP 2 | CCAGTGCCCA | CCAGUGCCCA |
| | (pos 8) | | |
| | Exon1 | GGAGGCACAG | GGAGGCACAG |
| | STOP 3 | ACCACTCCAC | ACCACUCCAC |
| | (pos 8) | | |
| | Exon1 | GGCACAGACA | GGCACAGACA |
| | STOP 4 | ACTCGACTGA | ACUCGACUGA |
| | (pos 5) | | |
| | Exon1 | GGAGGCACAG | GGAGGCACAG |
| | STOP 5 | ACCACTCCAC | ACCACUCCAC |
| | (pos 8) | | |
| | Exon1 | GCACAGACCA | GCACAGACCA |
| | STOP 6 | CTCAACCCAC | CUCAACCCAC |
| | (pos 4) | | |
| | Exon1 | GACCACTCAA | GACCACUCAA |
| | STOP 7 | CCCACAGGCC | CCCACAGGCC |
| | (pos 8) | | |
| | Exon1 | GACCACTCAA | GACCACUCAA |
| | STOP 8 | ACCACAGCCA | ACCACAGCCA |
| | (pos 8) | | |
| | Exon1 | GACCACTCAA | GACCACUCAA |
| | STOP 9 | CCCACAGCCA | CCCACAGCCA |
| | (pos 8) | | |
| | Exon1 | GGAGGCACAG | GGAGGCACAG |
| | STOP 10 | ACCACTCCAC | ACCACUCCAC |
| | (pos 8) | | |
| | Exon1 | GACCACTCAA | GACCACUCAA |
| | STOP 11 | CCAGCAGCCA | CCAGCAGCCA |
| | (pos 8) | | |
| CD3 | | TTCGTATCTG | UUCGUAUCUG |
| | | TAAAACCAAG | UAAAACCAAG |
| CD7 | | CCTACCTGTC | CCUACCUGUC |
| | | ACCAGGACCA | ACCAGGACCA |
| CD52 | | CTCTTACCTG | CUCUUACCUG |
| | | TACCATAACC | UACCAUAACC |
| PD1 | | CACCTACCTA | CACCUACCUA |
| | | AGAACCATCC | AGAACCAUCC |
| B2M | | ACTCACGCTG | ACUCACGCUG |
| | | GATAGCCTCC | GAUAGCCUCC |
| CD5 | | ACTCACCCAG | ACUCACCCAG |
| | | CATCCCCAGC | CAUCCCCAGC |
| CIITA | | CACTCACCTT | CACUCACCUU |
| | | AGCCTGAGCA | AGCCUGAGCA |
| CD2 | | CACGCACCTG | CACGCACCUG |
| | | GACAGCTGAC | GACAGCUGAC |

TABLE 8B

| | | gRNA | | | |
|---|---|---|---|---|---|
| | gRNA | tar- | Orienta- | Target | Predicted |
| Gene | Name | get | tion | Base(s) | Outcome |
| PDCD1 | Ex 1 SD | CAC | Antisense | C7 | splice |
| | | CTA | | | donor |
| | | CCT | | | distrup- |
| | | AAG | | | tion: |
| | | AAC | | | GT → AT |
| | | CAT | | | |
| | | CC | | | |
| PDCD1 | Ex 2 SA | GGA | Antisense | C6 | splice |
| | | GTC | | | donor |
| | | TGA | | | distrup- |
| | | GAG | | | tion: |
| | | ATG | | | AG → AA |
| | | GAG | | | |
| | | AG | | | |
| PDCD1 | Ex 3 SA | TTC | Antisense | C7 | splice |
| | | TCT | | | donor |
| | | CTG | | | distrup- |
| | | GAA | | | tion: |
| | | GGG | | | AG → AA |
| | | CAC | | | |
| | | AA | | | |
| PDCD1 | Ex 3 SD | GAC | Antisense | C8 | splice |
| | | GTT | | | donor |
| | | ACC | | | distrup- |
| | | TCG | | | tion: |
| | | TGC | | | GT → AT |
| | | GGC | | | |
| | | CC | | | |
| PDCD1 | Ex 4 SA | CCT | Antisense | C2 | splice |
| | | GCA | | | donor |
| | | GAG | | | distrup- |
| | | AAA | | | tion: |
| | | CAC | | | AG → AA |
| | | ACT | | | |
| | | TG | | | |
| PDCD1 | Ex 2 | GGG | Antisense | C7, | PmSTO |
| | pmSTOP | GTT | | C8 | P |
| | | CCA | | | Induction: |
| | | GGG | | | TGG |
| | | CCT | | | (Trp) → |
| | | GTC | | | TAG, |
| | | TG | | | TGA, |
| | | | | | TAA |
| PDCD1 | Ex 3 | CAG | Sense | C7 | splice |
| | pmSTOP_1 | TTC | | | donor |
| | | CAA | | | distrup- |
| | | ACC | | | tion: |
| | | CTG | | | CAA |
| | | GTG | | | (Gln) → |
| | | GT | | | TAA |
| PDCD1 | Ex 3 | GGA | Antisense | C5, | PmSTO |
| | pmSTOP_2 | CCC | | C6 | P |
| | | AGA | | | Induction: |
| | | CTA | | | TGG |
| | | GCA | | | (Trp) → |
| | | GCA | | | TAG, |
| | | CC | | | TGA, |
| | | | | | TAA |
| TRAC | Ex 1 SD | CTT | Antisense | C5 | splice |
| | | ACC | | | donor |
| | | TGG | | | distrup- |
| | | GCT | | | tion: |
| | | GGG | | | GT → AT |
| | | GAA | | | |
| | | GA | | | |
| TRAC | Ex 3 SA | TTC | Antisense | C8 | splice |
| | | GTA | | | donor |
| | | TCT | | | distrup- |
| | | GTA | | | tion: |
| | | AAA | | | AG → AA |
| | | CCA | | | |
| | | AG | | | |
| TRAC | Ex 3 | TTT | Sense | C4 | PmSTO |
| | pmSTOP_1 | CAA | | | P |
| | | AAC | | | Induc- |
| | | CTG | | | tion: |
| | | TCA | | | CAA |
| | | GTG | | | (Gln) → |
| | | AT | | | TAA |
| TRAC | Ex 3 | TTC | Sense | C3 | PmSTO |
| | pmSTOP_2 | AAA | | | P |
| | | ACC | | | Induc- |
| | | TGT | | | tion: |
| | | CAG | | | CAA |
| | | TGA | | | (Gln) → |
| | | TT | | | TAA |
| B2M | Ex 1 SD | ACT | Antisense | C6 | splice |
| | | CAC | | | donor |
| | | GCT | | | distrup- |
| | | GGA | | | tion: |
| | | TAG | | | GT → AT |
| | | CCT | | | |
| | | CC | | | |
| B2M | Ex 3 SA | TCG | Antisense | C6 | splice |
| | | ATC | | | donor |
| | | TAT | | | distrup- |
| | | GAA | | | tion: |
| | | AAA | | | AG → AA |
| | | GAC | | | |
| | | AG | | | |
| B2M | Ex 2 | CTT | Antisense | C7, | PmSTOP |
| | pmSTOP | ACC | | C8 | Induc- |
| | | CCA | | | tion: |
| | | CTT | | | TGG |
| | | AAC | | | (Trp) → |
| | | TAT | | | TAG, |
| | | CT | | | TGA, |
| | | | | | TAA |

TABLE 8C

| | | gRNA | gRNA |
|---|---|---|---|
| Gene | Description | Target | spacer |
| ACLY | Exon 1 SA | CCATC | CCAUC |
| | | GGCTC | GGCUC |
| | | GCGGC | GCGGC |
| | | GAGAA | GAGAA |
| | Exon 2 SA | CCTGT | CCUGU |
| | | CTGGG | CUGGG |
| | | AGAGA | AGAGA |
| | | GAAGC | GAAGC |
| | Exon 2 SD 1 | GCTCA | GCUCA |
| | | CCTGG | CCUGG |
| | | CTGAG | CUGAG |
| | | CAGCC | CAGCC |
| | Exon 2 SD 2 | CTCAC | CUCAC |
| | | CTGGC | CUGGC |
| | | TGAGC | UGAGC |
| | | AGCCA | AGCCA |
| | Exon 3 SA | ACCAA | ACCAA |
| | | GTTCT | GUUCU |
| | | GGAAC | GGAAC |
| | | AAAAG | AAAAG |
| | Exon 4 SA 1 | GCCAA | GCCAA |
| | | CCTAC | CCUAC |
| | | AGAAA | AGAAA |
| | | AATTG | AAUUG |
| | Exon 4 SA 2 | CCAAC | CCAAC |
| | | CTACA | CUACA |
| | | GAAAA | GAAAA |
| | | ATTGA | AUUGA |
| | Exon 5 SA | AGCCT | AGCCU |
| | | TGCAG | UGCAG |
| | | GTGAA | GUGAA |
| | | GAGAC | GAGAC |
| | Exon 5 SD 1 | CTCAA | CUCAA |
| | | CTCTT | CUCUU |
| | | TCTTG | UCUUG |
| | | TCTTC | UCUUC |
| | Exon 5 SD 2 | TCAAC | UCAAC |
| | | TCTTT | UCUUU |
| | | CTTGT | CUUGU |
| | | CTTCA | CUUCA |
| | Exon 7 SA | CACTA | CACUA |
| | | CTTCA | CUUCA |
| | | AGGGG | AGGGG |
| | | AGCAG | AGCAG |
| | Exon 12 SD | ACCTA | ACCUA |
| | | CCGAT | CCGAU |
| | | GTGCT | GUGCU |
| | | CCCGC | CCCGC |
| | Exon 13 SA | CTGGC | CUGGC |
| | | GTCTG | GUCUG |
| | | GGGTG | GGGUG |
| | | AGATA | AGAUA |
| | Exon 13 SD | GAGTT | GAGUU |
| | | ACCTT | ACCUU |
| | | GTGGC | GUGGC |
| | | ATGGC | AUGGC |
| | Exon 14 SD | ATCCT | AUCCU |
| | | ACCTT | ACCUU |
| | | GCAGG | GCAGG |
| | | GATCT | GAUCU |
| | Exon 15 SD | TCACG | UCACG |
| | | TGAAA | UGAAA |
| | | GGGTA | GGGUA |
| | | GACCA | GACCA |
| | Exon 16 SD | ATCTA | AUCUA |
| | | CCTGG | CCUGG |
| | | GCATA | GCAUA |
| | | GTTCA | GUUCA |
| | Exon 18 SD | TGATT | UGAUU |
| | | ACCTG | ACCUG |
| | | TCCCC | UCCCC |
| | | ACCAA | ACCAA |
| | Exon 20 SA | CCCCA | CCCCA |
| | | ATCTG | AUCUG |
| | | CCAAG | CCAAG |
| | | GAATG | GAAUG |
| | Exon 20 SD | CCATA | CCAUA |
| | | CCTCA | CCUCA |
| | | GAGGA | GAGGA |
| | | GAACA | GAACA |
| | Exon 23 SA | CAAGC | CAAGC |
| | | TCCTG | UCCUG |
| | | GGCAG | GGCAG |
| | | AGATG | AGAUG |
| | Exon 26 SA | TTATC | UUAUC |
| | | TAGAA | UAGAA |
| | | ATGAA | AUGAA |
| | | CCCAA | CCCAA |
| ADORA2A | Exon 1 ATG | TGGGC | UGGGC |
| | | ATGGC | AUGGC |
| | | CACAG | CACAG |
| | | ACGAC | ACGAC |
| | Exon 1 SD | CTGCT | CUGCU |
| | | CACCG | CACCG |
| | | GAGCG | GAGCG |
| | | GGATG | GGAUG |
| | Exon 2 Stop 1 | CAGTT | CAGUU |
| | | GTTCC | GUUCC |
| | | AACCT | AACCU |
| | | AGCAT | AGCAU |
| | Exon 2 STOP 2 | CACTC | CACUC |
| | | CCAGG | CCAGG |
| | | GCTGC | GCUGC |
| | | GGGGA | GGGGA |
| | Exon 2 STOP 3 | CCACT | CCACU |
| | | CCCAG | CCCAG |
| | | GGCTG | GGCUG |
| | | CGGGG | CGGGG |
| | Exon 2 STOP 4 | GCGAC | GCGAC |
| | | GACAG | GACAG |
| | | CTGAA | CUGAA |
| | | GCAGA | GCAGA |
| | Exon 2 STOP 5 | GGAGA | GGAGA |
| | | GCCAG | GCCAG |
| | | CCTCT | CCUCU |
| | | GCCGG | GCCGG |
| | Exon 2 STOP 6 | ACATG | ACAUG |
| | | AGCCA | AGCCA |
| | | GAGAG | GAGAG |
| | | GGGCG | GGGCG |
| | Exon 2 STOP 7 | GAGGC | GAGGC |
| | | AGCAA | AGCAA |
| | | GAACC | GAACC |
| | | TTTCA | UUUCA |
| | Exon 2 STOP 8 | TGGCC | UGGCC |
| | | CACAC | CACAC |
| | | TCCTG | UCCUG |
| | | GCGGG | GCGGG |
| | Exon 2 STOP 9 | CGTTG | CGUUG |
| | | GCCCA | GCCCA |
| | | CACTC | CACUC |
| | | CTGGC | CUGGC |
| | Exon 2 STOP 10 | CTGGG | CUGGG |
| | | ACTCT | ACUCU |
| | | TGGGC | UGGGC |
| | | ACTCC | ACUCC |
| AXL | Exon 2 SA 1 | TGCGT | UGCGU |
| | | GCCTG | GCCUG |
| | | GAGGG | GAGGG |
| | | GAGAT | GAGAU |
| | Exon 2 SA 2 | CTGCG | CUGCG |
| | | TGCCT | UGCCU |
| | | GGAGG | GGAGG |
| | | GGAGA | GGAGA |
| | Exon 3 SA | GGTGA | GGUGA |
| | | TTCTG | UUCUG |
| | | ACAGG | ACAGG |
| | | GCAAG | GCAAG |
| | Exon 4 SA | AAGCC | AAGCC |
| | | TAGCG | UAGCG |
| | | GGGTG | GGGUG |
| | | GGCAG | GGCAG |
| | Exon 4 SD | CGGAC | CGGAC |
| | | TCACC | UCACC |
| | | TGGAA | UGGAA |
| | | CATGC | CAUGC |
| | Exon 5 SA 1 | AGCCC | AGCCC |
| | | TAGGG | UAGGG |
| | | AGTCA | AGUCA |
| | | TATGA | UAUGA |
| | Exon 5 SA 2 | CAGCC | CAGCC |
| | | CTAGG | CUAGG |
| | | GAGTC | GAGUC |
| | | ATATG | AUAUG |
| | Exon 6 SD 1 | TCTCA | UCUCA |
| | | CCTGC | CCUGC |
| | | AGGGT | AGGGU |
| | | GCAGT | GCAGU |
| | Exon 6 SD 2 | GTCTC | GUCUC |
| | | ACCTG | ACCUG |
| | | CAGGG | CAGGG |
| | | TGCAG | UGCAG |
| | Exon 7 SA 1 | CACAG | CACAG |
| | | CCTGA | CCUGA |
| | | GGAGA | GGAGA |
| | | GGCAA | GGCAA |
| | Exon 7 SA 2 | GCACA | GCACA |
| | | GCCTG | GCCUG |
| | | AGGAG | AGGAG |
| | | AGGCA | AGGCA |
| | Exon 8 SA 1 | GCACT | GCACU |
| | | GGAGG | GGAGG |
| | | ACAGG | ACAGG |
| | | GAAGA | GAAGA |
| | Exon 8 SA 2 | GGCAC | GGCAC |
| | | TGGAG | UGGAG |
| | | GACAG | GACAG |
| | | GGAAG | GGAAG |
| | Exon 8 SD | CACCC | CACCC |
| | | ACCTC | ACCUC |
| | | TGGGG | UGGGG |
| | | TGTCC | UGUCC |
| | Exon 9 SA | GCACC | GCACC |
| | | TAGGA | UAGGA |
| | | GGTCC | GGUCC |
| | | AGAAG | AGAAG |
| | Exon 10 SD | CCCTT | CCCUU |
| | | ACCCA | ACCCA |
| | | GCTGG | GCUGG |
| | | TGGAC | UGGAC |
| | Exon 11 SA | CTTCA | CUUCA |
| | | CTATC | CUAUC |
| | | AGGGG | AGGGG |
| | | GTATG | GUAUG |
| | Exon 12 SA | TCACT | UCACU |
| | | TACAG | UACAG |
| | | GTAGC | GUAGC |
| | | TTCAG | UUCAG |
| | Exon 13 SA 1 | GTTCA | GUUCA |
| | | CTGCA | CUGCA |
| | | TGCAA | UGCAA |
| | | GGTTG | GGUUG |
| | Exon 13 SA 2 | TGTTC | UGUUC |
| | | ACTGC | ACUGC |
| | | ATGCA | AUGCA |
| | | AGGTT | AGGUU |
| | Exon 13 SA 3 | CTGTT | CUGUU |
| | | CACTG | CACUG |
| | | CATGC | CAUGC |
| | | AAGGT | AAGGU |
| | Exon 14 SA 1 | CTCTC | CUCUC |
| | | CTGTG | CUGUG |
| | | GGGGG | GGGGG |
| | | CCAGA | CCAGA |
| | Exon 14 SA 2 | ACTCT | ACUCU |
| | | CCTGT | CCUGU |
| | | GGGGG | GGGGG |
| | | GCCAG | GCCAG |
| | Exon 15 SA | GCAAC | GCAAC |
| | | TTGAG | UUGAG |
| | | GGAGA | GGAGA |
| | | GAGAA | GAGAA |
| | Exon 17 SA | AGGTA | AGGUA |
| | | CTGGG | CUGGG |
| | | GAGCC | GAGCC |
| | | AAGGC | AAGGC |
| | Exon 18 SD | ACCTA | ACCUA |
| | | CCACA | CCACA |
| | | TCGCT | UCGCU |
| | | CTTGC | CUUGC |
| | Exon 19 SA 1 | GGACC | GGACC |
| | | ACTGT | ACUGU |
| | | GAGGG | GAGGG |
| | | GCAGA | GCAGA |
| | Exon 19 SA 2 | AGGAC | AGGAC |
| | | CACTG | CACUG |
| | | TGAGG | UGAGG |
| | | GGCAG | GGCAG |
| | Exon 20 SA | ATACC | AUACC |
| | | TAGGG | UAGGG |
| | | CAGCA | CAGCA |
| | | AAATG | AAAUG |
| BATF | Exon 1 ATG | GAGGC | GAGGC |
| | | ATGGC | AUGGC |
| | | TGAAA | UGAAA |
| | | TCTTC | UCUUC |
| | Exon 1 SD 1 | TCTAC | UCUAC |
| | | CTGTT | CUGUU |
| | | TGCCA | UGCCA |
| | | GGGGG | GGGGG |
| | Exon 1 SD2 | GACTC | GACUC |
| | | TACCT | UACCU |
| | | GTTTG | GUUUG |
| | | CCAGG | CCAGG |
| | Exon 2 SA 1 | AGTCC | AGUCC |
| | | TGGGA | UGGGA |
| | | AGCAG | AGCAG |
| | | AGACG | AGACG |
| | Exon 2 SA 2 | GAGTC | GAGUC |
| | | CTGGG | CUGGG |
| | | AAGCA | AAGCA |
| | | GAGAC | GAGAC |
| | Exon 2 SA 3 | TGAGT | UGAGU |
| | | CCTGG | CCUGG |
| | | GAAGC | GAAGC |
| | | AGAGA | AGAGA |
| | Exon 2 SD | ACTTA | ACUUA |
| | | CCAGG | CCAGG |
| | | TGCAG | UGCAG |
| | | GGTGT | GGUGU |
| BCL2L11 | Exon 1 STOP 1 | GGTAG | GGUAG |
| | | ACAAT | ACAAU |
| | | TGCAG | UGCAG |
| | | CCTG | CCUG |
| | Exon 1 STOP 2 | GCCTC | GCCUC |
| | | CCCAG | CCCAG |
| | | CTCAG | CUCAG |
| | | ACCTG | ACCUG |
| | Exon 1 STOP 3 | TCCCT | UCCCU |
| | | ACAGA | ACAGA |
| | | CAGAG | CAGAG |
| | | CCACA | CCACA |
| | Exon 1 STOP 4 | GAGCC | GAGCC |
| | | ACAAG | ACAAG |
| | | GTAAT | GUAAU |
| | | CCTGA | CCUGA |
| | Exon 3 STOP 1 | GCCCA | GCCCA |
| | | AGAGT | AGAGU |
| | | TGCGG | UGCGG |
| | | CGTAT | CGUAU |
| | Exon 4 SA | AAAAT | AAAAU |
| | | ACCTG | ACCUG |
| | | AAACA | AAACA |
| | | ACAAA | ACAAA |
| CAMK2D | Exon 6 SA 1 | CAATG | CAAUG |
| | | ACTGC | ACUGC |
| | | AAAGA | AAAGA |
| | | TACAA | UACAA |
| | Exon 6 SA 2 | ACAAT | ACAAU |
| | | GACTG | GACUG |
| | | CAAAG | CAAAG |
| | | ATACA | AUACA |
| | Exon 7 SA 3 | AATGA | AAUGA |
| | | CTGCA | CUGCA |
| | | TGCAA | UGCAA |
| | | ACACC | ACACC |
| | Exon 7 SA 2 | ATGAC | AUGAC |
| | | TGCAT | UGCAU |
| | | GCAAA | GCAAA |
| | | CACCA | CACCA |
| | Exon 7 SA 1 | TGACT | UGACU |
| | | GCATG | GCAUG |
| | | CAAAC | CAAAC |
| | | ACCAG | ACCAG |
| | Exon 7 SD | TACTC | UACUC |
| | | ACCTT | ACCUU |
| | | CAGGT | CAGGU |
| | | CCCGA | CCCGA |
| | Exon 8 SD | ACTCA | ACUCA |
| | | CCAAA | CCAAA |
| | | CCACG | CCACG |
| | | CCTGC | CCUGC |
| | Exon 14 SD | TAACT | UAACU |
| | | TACCT | UACCU |
| | | TTACT | UUACU |
| | | CCATC | CCAUC |
| | Exon 16 SD | AGGTA | AGGUA |
| | | TACCA | UACCA |
| | | GCGCT | GCGCU |
| | | GGGGT | GGGGU |
| | Exon 17 SD 1 | TGAAT | UGAAU |
| | | ACCTT | ACCUU |
| | | GTTTC | GUUUC |
| | | CATCA | CAUCA |
| | Exon 17 SD 2 | CTGAA | CUGAA |
| | | TACCT | UACCU |
| | | TGTTT | UGUUU |
| | | CCATC | CCAUC |
| | Exon 19 SA | TCGTG | UCGUG |
| | | CTAAA | CUAAA |
| | | GGCAA | GGCAA |
| | | AAATA | AAAUA |
| cAMP | Exon 1 SD 1 | AGCTC | AGCUC |
| | | ACCAT | ACCAU |
| | | CGTGG | CGUGG |
| | | GCCTG | GCCUG |
| | Exon 1 SD 2 | AAGCT | AAGCU |
| | | CACCA | CACCA |
| | | TCGTG | UCGUG |
| | | GGCCT | GGCCU |
| | Exon 1 SD 3 | AAAGC | AAAGC |
| | | TCACC | UCACC |
| | | ATCGT | AUCGU |
| | | GGGCC | GGGCC |
| | Exon 2 SA | ATCCT | AUCCU |
| | | AGTCA | AGUCA |
| | | GAGGA | GAGGA |
| | | GGAAA | GGAAA |
| | Exon 3 SA | GTTAT | GUUAU |
| | | CCTGG | CCUGG |
| | | GGTTG | GGUUG |
| | | TGTAC | UGUAC |
| CASP8 | Exon “3” SA | GAACC | GAACC |
| | | TTCAA | UUCAA |
| | | AGGAC | AGGAC |
| | | CAAGA | CAAGA |
| | Exon 1 SD | TCACC | UCACC |
| | | CGCTC | CGCUC |
| | | CACCC | CACCC |
| | | TTTCC | UUUCC |
| | Exon 2 SA | ATAAT | AUAAU |
| | | CTAAG | CUAAG |
| | | TCAAA | UCAAA |
| | | ATAAA | AUAAA |
| | Exon 2.5 SA | AGTCC | AGUCC |
| | | ATCTT | AUCUU |
| | | TTTAA | UUUAA |
| | | AAGGC | AAGGC |
| | Exon 3 SA | CATGA | CAUGA |
| | | CCCTG | CCCUG |
| | | TGGTG | UGGUG |
| | | GGAAA | GGAAA |
| | Exon 5 SD | TTACC | UUACC |
| | | ATTTG | AUUUG |
| | | AAAAT | AAAAU |
| | | TCATC | UCAUC |
| CCR5 | Exon 1 STOP | CATAC | CAUAC |
| | | AGTCA | AGUCA |
| | | GTATC | GUAUC |
| | | AATTC | AAUUC |
| | Exon 1 STOP 2 | GGTGT | GGUGU |
| | | CGAAA | CGAAA |
| | | TGAGA | UGAGA |
| | | AGAAG | AGAAG |
| | Exon 1 STOP 3 | ATGCA | AUGCA |
| | | GGTGA | GGUGA |
| | | CAGAG | CAGAG |
| | | ACTCT | ACUCU |
| | Exon 1 STOP 4 | TGGGG | UGGGG |
| | | AGCAG | AGCAG |
| | | GAAAT | GAAAU |
| | | ATCTG | AUCUG |
| CD2 | Ex3 SD | CACGC | CACGC |
| | (pos 8) | ACCTG | ACCUG |
| | | GACAG | GACAG |
| | | CTGAC | CUGAC |
| | Ex3 STOP1 | TCTCA | UCUCA |
| | (Pos 4) | AAACC | AAACC |
| | | AAAGA | AAAGA |
| | | TCTCC | UCUCC |
| | Ex3 STOP2 | CAACA | CAACA |
| | (Pos 6) | CAACC | CAACC |
| | | CTGAC | CUGAC |
| | | CTGTG | CUGUG |
| | Ex4 STOP | AAACA | AAACA |
| | (pos 4) | GAGGA | GAGGA |
| | | GTCGG | GUCGG |
| | | AGAAA | AGAAA |
| | Ex4 STOP2 | TCACC | UCACC |
| | (Pos 5) | AAAAG | AAAAG |
| | | GAAAA | GAAAA |
| | | AACAG | AACAG |
| | Ex5 STOP | ACACA | ACACA |
| | (pos 4) | AGTTC | AGUUC |
| | | ACCAG | ACCAG |
| | | CAGAA | CAGAA |
| | Ex5 STOP | GTTCA | GUUCA |
| | (pos 4) | GCCAA | GCCAA |
| | | AACCT | AACCU |
| | | CCCCA | CCCCA |
| | Exon 2 STOP | CTTGG | CUUGG |
| | (pos 8) | GTCAG | GUCAG |
| | | GACAT | GACAU |
| | | CAACT | CAACU |
| | Exon 2 STOP | CGATG | CGAUG |
| | (pos 8) | ATCAG | AUCAG |
| | | GATAT | GAUAU |
| | | CTACA | CUACA |
| CD3D | Exon 1 SD 1 | AGCCT | AGCCU |
| | | TACCT | UACCU |
| | | TGCGA | UGCGA |
| | | GAGAA | GAGAA |
| | Exon 1 SD 2 | TAGCC | UAGCC |
| | | TTACC | UUACC |
| | | TTGCG | UUGCG |
| | | AGAGA | AGAGA |
| | Exon 1 STOP | TCGCA | UCGCA |
| | | AGGTA | AGGUA |
| | | AGGCT | AGGCU |
| | | ACTCC | ACUCC |
| | Exon 3 SA | GGCAC | GGCAC |
| | | ACTGT | ACUGU |
| | | GGGGG | GGGGG |
| | | AAGGG | AAGGG |
| | Exon 3 STOP | GTGCC | GUGCC |
| | | AGAGC | AGAGC |
| | | TGTGT | UGUGU |
| | | GGAGC | GGAGC |
| | Exon 4 STOP 1 | CCGAC | CCGAC |
| | | ACACA | ACACA |
| | | AGCTC | AGCUC |
| | | TGTTG | UGUUG |
| | Exon 4 STOP 2 | GGTCT | GGUCU |
| | | ATCAG | AUCAG |
| | | GTGAG | GUGAG |
| | | CGTTG | CGUUG |
| | Exon 5 STOP | GATGC | GAUGC |
| | | TCAGT | UCAGU |
| | | ACAGC | ACAGC |
| | | CACCT | CACCU |
| CD3E | Exon 1 ATG | CCGAC | CCGAC |
| | | TGCAT | UGCAU |
| | | CTTTG | CUUUG |
| | | TTTCA | UUUCA |
| | Exon 1 SD | ACTCA | ACUCA |
| | | CCTGA | CCUGA |
| | | TAAGA | UAAGA |
| | | GGCAG | GGCAG |
| | Exon 4 SA | TACCA | UACCA |
| | | CCTGA | CCUGA |
| | | AAATG | AAAUG |
| | | AAAAA | AAAAA |
| | Exon 4 STOP | ACACA | ACACA |
| | | GACAC | GACAC |
| | | GTGAG | GUGAG |
| | | TTTAT | UUUAU |
| | Exon 5 SA 1 | TATAT | UAUAU |
| | | GCTGG | GCUGG |
| | | GGAGA | GGAGA |
| | | AAGAA | AAGAA |
| | Exon 5 SA 2 | TTATA | UUAUA |
| | | TGCTG | UGCUG |
| | | GGGAG | GGGAG |
| | | AAAGA | AAAGA |
| | Exon 5 SD | CTGGA | CUGGA |
| | | TTACC | UUACC |
| | | TCTTG | UCUUG |
| | | CCCTC | CCCUC |
| | Exon 6 SA 1 | ACACT | ACACU |
| | | GTGGG | GUGGG |
| | | GGGTG | GGGUG |
| | | GGGTG | GGGUG |
| | Exon 6 SA 2 | CACAC | CACAC |
| | | TGTGG | UGUGG |
| | | GGGGT | GGGGU |
| | | GGGGT | GGGGU |
| | Exon 6 SA 3 | ACACA | ACACA |
| | | CTGTG | CUGUG |
| | | GGGGG | GGGGG |
| | | TGGGG | UGGGG |
| | Exon 7 SA 1 | TTGTC | UUGUC |
| | | CTGCG | CUGCG |
| | | GAGGA | GAGGA |
| | | AGGAG | AGGAG |
| | Exon 7 SA 2 | TTTGT | UUUGU |
| | | CCTGC | CCUGC |
| | | GGAGG | GGAGG |
| | | AAGGA | AAGGA |
| | Exon 7 SA 3 | TTTTG | UUUUG |
| | | TCCTG | UCCUG |
| | | CGGAG | CGGAG |
| | | GAAGG | GAAGG |
| | Exon 7 SD 1 | GTTAC | GUUAC |
| | | CTCAT | CUCAU |
| | | AGTCT | AGUCU |
| | | GGGTT | GGGUU |
| | Exon 7 SD 2 | CGTTA | CGUUA |
| | | CCTCA | CCUCA |
| | | TAGTC | UAGUC |
| | | TGGGT | UGGGU |
| CD3G | Exon 1 STOP 1 | CATGG | CAUGG |
| | | AACAG | AACAG |
| | | GGGAA | GGGAA |
| | | GGGCC | GGGCC |
| | Exon 1 STOP 2 | CTTCA | CUUCA |
| | | AGGTA | AGGUA |
| | | AGGGC | AGGGC |
| | | CTACT | CUACU |
| | Exon 2 SD 1 | TCTCC | UCUCC |
| | | TACCT | UACCU |
| | | TTGAT | UUGAU |
| | | TGACT | UGACU |
| | Exon 2 SD 2 | TTCTC | UUCUC |
| | | CTACC | CUACC |
| | | TTTGA | UUUGA |
| | | TTGAC | UUGAC |
| | Exon 2 STOP | TGGCC | UGGCC |
| | | CAGTC | CAGUC |
| | | AATCA | AAUCA |
| | | AAGGT | AAGGU |
| | Exon 3 SD | ACATA | ACAUA |
| | | CTTCT | CUUCU |
| | | GTAAT | GUAAU |
| | | ACACT | ACACU |
| | Exon 3 STOP 1 | TGACT | UGACU |
| | | ATCAA | AUCAA |
| | | GAAGA | GAAGA |
| | | TGGTT | UGGUU |
| | Exon 3 STOP 2 | TTTAA | UUUAA |
| | | ACCAT | ACCAU |
| | | GTGAT | GUGAU |
| | | ATTTT | AUUUU |
| | Exon 4 STOP | CTCTT | CUCUU |
| | | CCATT | CCAUU |
| | | GGGTA | GGGUA |
| | | CATAA | CAUAA |
| | Exon 5 STOP | TGACC | UGACC |
| | | AGCTC | AGCUC |
| | | TACCA | UACCA |
| | | GGTAA | GGUAA |
| | Exon 7 STOP 1 | GACCA | GACCA |
| | | GTACA | GUACA |
| | | GCCAC | GCCAC |
| | | CTTCA | CUUCA |
| | Exon 7 STOP 2 | ACCTT | ACCUU |
| | | CAAGG | CAAGG |
| | | AAACC | AAACC |
| | | AGTTG | AGUUG |
| CD4 | Exon 1 ATG | GGTTC | GGUUC |
| | | ATTGT | AUUGU |
| | | GGCCT | GGCCU |
| | | TGCCG | UGCCG |
| | Exon 2 SA | GAGCG | GAGCG |
| | | CTAAG | CUAAG |
| | | TGGAA | UGGAA |
| | | AAGAA | AAGAA |
| | Exon 2 SD | AACCC | AACCC |
| | | TACCT | UACCU |
| | | TTAGT | UUAGU |
| | | TAAGA | UAAGA |
| | Exon 5 SA | GGCAG | GGCAG |
| | | TCACT | UCACU |
| | | GTGGA | GUGGA |
| | | GGGAA | GGGAA |
| | Exon 6 SA | TGGAA | UGGAA |
| | | AGCTG | AGCUG |
| | | GAGGT | GAGGU |
| | | GGGAA | GGGAA |
| | Exon 6 SD | CCTCA | CCUCA |
| | | CCTCT | CCUCU |
| | | CATCA | CAUCA |
| | | CCACC | CCACC |
| | Exon 7 SA | AGTGG | AGUGG |
| | | CTGCA | CUGCA |
| | | GAGGA | GAGGA |
| | | ACGAG | ACGAG |
| | Exon 10 SA | GCGCT | GCGCU |
| | | GTCCA | GUCCA |
| | | GGGAC | GGGAC |
| | | AAGAA | AAGAA |
| | Exon 10 SD | TCCTT | UCCUU |
| | | ACTGA | ACUGA |
| | | GGACA | GGACA |
| | | CTGGC | CUGGC |
| | short alt | CCATC | CCAUC |
| | exon 2 SA | TGGAG | UGGAG |
| | | CTTAG | CUUAG |
| | | GGTCC | GGUCC |
| | Short CD4 ATG | GGTTG | GGUUG |
| | | GCATG | GCAUG |
| | | TGGAG | UGGAG |
| | | GCAGC | GCAGC |
| CD5 | Ex2 STOP 2 | GGGTC | GGGUC |
| | (pos 6) | ATACC | AUACC |
| | | AGCTG | AGCUG |
| | | AGCCG | AGCCG |
| | Ex3 SA | TGGAA | UGGAA |
| | (pos 8) | ATCTG | AUCUG |
| | | GGGGT | GGGGU |
| | | CAGAA | CAGAA |
| | Ex3 SD | GTTAC | GUUAC |
| | (pos 9) | CCACC | CCACC |
| | | TAAGC | UAAGC |
| | | AGGTC | AGGUC |
| | Ex3 STOP | TCTGC | UCUGC |
| | (pos 6) | CAGCG | CAGCG |
| | | GCTGA | GCUGA |
| | | ACTGT | ACUGU |
| | Ex3 STOP | CTGCC | CUGCC |
| | (pos 5) | AGCGG | AGCGG |
| | | CTGAA | CUGAA |
| | | CTGTG | CUGUG |
| | Ex3 STOP | CCTCC | CCUCC |
| | (pos 5/6) | CACTG | CACUG |
| | | CTTGG | CUUGG |
| | | AGCTC | AGCUC |
| | Ex3 STOP | GAAGT | GAAGU |
| | (pos 8) | GCCAG | GCCAG |
| | | GGCCA | GGCCA |
| | | GCTGG | GCUGG |
| | Ex3 STOP | CCATG | CCAUG |
| | (pos 8/9) | TGCCA | UGCCA |
| | | TCCGT | UCCGU |
| | | CCTTG | CCUUG |
| | Ex3 STOP | TTTGC | UUUGC |
| | (pos 9) | AGCCA | AGCCA |
| | | GAGCT | GAGCU |
| | | GGGGC | GGGGC |
| | Ex4 SA | GGTTC | GGUUC |
| | (pos 5) | TGCAA | UGCAA |
| | | TGAGA | UGAGA |
| | | CACTC | CACUC |
| | Ex4 STOP | CTCCA | CUCCA |
| | (pos 4) | GAGCC | GAGCC |
| | | CACAG | CACAG |
| | | GTAAG | GUAAG |
| | Ex4 STOP2 | ACCAC | ACCAC |
| | (Pos 5) | AACTC | AACUC |
| | | CAGAG | CAGAG |
| | | CCCAC | CCCAC |
| | Ex5 SA | GAGCT | GAGCU |
| | (pos 4) | AGGAG | AGGAG |
| | | AGGAG | AGGAG |
| | | AGAGC | AGAGC |
| | Ex5 SD | CTCAC | CUCAC |
| | (pos 9) | TTACC | UUACC |
| | | TGAGC | UGAGC |
| | | AAAGG | AAAGG |
| | Ex5 STOP | CTGCA | CUGCA |
| | (pos 5) | GCTGG | GCUGG |
| | | TGGCA | UGGCA |
| | | CAGTC | CAGUC |
| | Ex5 STOP | GATCT | GAUCU |
| | (pos 7) | TCCAT | UCCAU |
| | | TGGAT | UGGAU |
| | | TGGCA | UGGCA |
| | Ex5 STOP | TGAGG | UGAGG |
| | (pos 8) | CCCAG | CCCAG |
| | | GACAA | GACAA |
| | | GACCC | GACCC |
| | Ex6 SA | AAACC | AAACC |
| | (pos 5) | TGAGA | UGAGA |
| | | GGGGA | GGGGA |
| | | AGCAA | AGCAA |
| | Ex6 STOP | CTCCC | CUCCC |
| | (pos 4/5) | ACCGC | ACCGC |
| | | AGCGA | AGCGA |
| | | GCTCC | GCUCC |
| | Ex6 STOP | TTTCC | UUUCC |
| | (pos 5) | AGCCC | AGCCC |
| | | AAGGT | AAGGU |
| | | GCAGA | GCAGA |
| | Ex6 STOP | GGTGC | GGUGC |
| | (pos 5) | AGAGC | AGAGC |
| | | CGTCT | CGUCU |
| | | GGTGG | GGUGG |
| | Ex6 STOP | AGGTG | AGGUG |
| | (pos 6) | CAGAG | CAGAG |
| | | CCGTC | CCGUC |
| | | TGGTG | UGGUG |
| | Ex6 STOP | TCCTA | UCCUA |
| | (pos 7) | TCGAG | UCGAG |
| | | TGCTG | UGCUG |
| | | GACGC | GACGC |
| | Ex6 STOP | AAGGT | AAGGU |
| | (pos 7) | GCAGA | GCAGA |
| | | GCCGT | GCCGU |
| | | CTGGT | CUGGU |
| | Ex6 STOP | CAAGG | CAAGG |
| | (pos 8) | TGCAG | UGCAG |
| | | AGCCG | AGCCG |
| | | TCTGG | UCUGG |
| | Ex6 STOP | GGGCT | GGGCU |
| | (pos 8/9) | GCCCA | GCCCA |
| | | CTGAG | CUGAG |
| | | CCCCC | CCCCC |
| | Ex6 STOP | AGGTG | AGGUG |
| | (pos 9) | CGCCA | CGCCA |
| | | GGGGG | GGGGG |
| | | CTCAG | CUCAG |
| | Ex7 STOP | GGCCA | GGCCA |
| | (pos 4) | GGATC | GGAUC |
| | | CAAAC | CAAAC |
| | | CCCGC | CCCGC |
| | Ex8 STOP | CGCCA | CGCCA |
| | (pos 4) | GTGGA | GUGGA |
| | | TTGGC | UUGGC |
| | | CCAAC | CCAAC |
| | Ex8 STOP | GCGCC | GCGCC |
| | (pos 5) | AGTGG | AGUGG |
| | | ATTGG | AUUGG |
| | | CCCAA | CCCAA |
| | Ex8 STOP | AAGAA | AAGAA |
| | (pos 7) | GCAGC | GCAGC |
| | | GCCAG | GCCAG |
| | | TGGAT | UGGAU |
| | Ex9 SD | GCTTA | GCUUA |
| | (pos 6) | CCTGG | CCUGG |
| | | ATAAG | AUAAG |
| | | CTGAC | CUGAC |
| | Ex9 SD1 | AAAGA | AAAGA |
| | (Pos 8) | CACTG | CACUG |
| | | GGCAG | GGCAG |
| | | ATGGT | AUGGU |
| | Ex10 SA | TTCCA | UUCCA |
| | (pos 9) | GAGCT | GAGCU |
| | | GGGGA | GGGGA |
| | | AAGAA | AAGAA |
| | Exon 1 SD | ACTCA | ACUCA |
| | (pos 6) | CCCAG | CCCAG |
| | | CATCC | CAUCC |
| | | CCAGC | CCAGC |
| | Exon 2 SA | AGCGA | AGCGA |
| | (pos 6) | CTGCA | CUGCA |
| | | GAAAG | GAAAG |
| | | AAGAG | AAGAG |
| | Exon 2 STOP | CATAC | CAUAC |
| | (pos 5/6) | CAGCT | CAGCU |
| | | GAGCC | GAGCC |
| | | GTCCG | GUCCG |
| CD8A | Exon 1 ATG | AAGGC | AAGGC |
| | | CATGA | CAUGA |
| | | CGCGC | CGCGC |
| | | TCCCC | UCCCC |
| | Exon 1 SD | TCACG | UCACG |
| | | GAGCA | GAGCA |
| | | GCAAG | GCAAG |
| | | GCCAG | GCCAG |
| | Exon 2 SD | CGCGG | CGCGG |
| | | ACCTG | ACCUG |
| | | GCAGG | GCAGG |
| | | AAGAC | AAGAC |
| | Exon 3 SD | TCACC | UCACC |
| | | TGCGC | UGCGC |
| | | CCCCC | CCCCC |
| | | GCCGC | GCCGC |
| | Exon 4 SD 1 | CTTAC | CUUAC |
| | | TGTGG | UGUGG |
| | | TTGCA | UUGCA |
| | | GTAAA | GUAAA |
| | Exon 4 SD 2 | ACTTA | ACUUA |
| | | CTGTG | CUGUG |
| | | GTTGC | GUUGC |
| | | AGTAA | AGUAA |
| CD38 | Exon 1 ATG 1 | TTGGC | UUGGC |
| | | CATAG | CAUAG |
| | | GGCTC | GGCUC |
| | | CAGGC | CAGGC |
| | Exon 1 ATG 2 | GTTGG | GUUGG |
| | | CCATA | CCAUA |
| | | GGGCT | GGGCU |
| | | CCAGG | CCAGG |
| | Exon 1 STOP | GCGCC | GCGCC |
| | | AGCAG | AGCAG |
| | | TGGAG | UGGAG |
| | | CGGTC | CGGUC |
| | Exon 2 SD | AATTA | AAUUA |
| | | CCTTG | CCUUG |
| | | TTGCA | UUGCA |
| | | AGGTA | AGGUA |
| | Exon 2 STOP | CTATC | CUAUC |
| | | AGCCA | AGCCA |
| | | CTAAT | CUAAU |
| | | GAAGT | GAAGU |
| | Exon 3 STOP 1 | CTGCT | CUGCU |
| | | CCAAA | CCAAA |
| | | GAAGA | GAAGA |
| | | ATCTA | AUCUA |
| | Exon 4 STOP 1 | ACTAT | ACUAU |
| | | CAATC | CAAUC |
| | | TTGCC | UUGCC |
| | | CAGAC | CAGAC |
| | Exon 4 STOP 2 | TTT1C | UUUUC |
| | | CAGAA | CAGAA |
| | | TACTG | UACUG |
| | | AAACA | AAACA |
| | Exon 4 STOP 3 | GTTTT | GUUUU |
| | | CCAGA | CCAGA |
| | | ATACT | AUACU |
| | | GAAAC | GAAAC |
| | Exon 7 SD | TTACC | UUACC |
| | | TGTAG | UGUAG |
| | | ATATT | AUAUU |
| | | CTTGC | CUUGC |
| CD70 | Ex1 SD | CTCAC | CUCAC |
| | (pos 6) | CCCAA | CCCAA |
| | | GTGAC | GUGAC |
| | | TCGAG | UCGAG |
| | Ex1 STOP | GTGCA | GUGCA |
| | (pos 8) | TCCAG | UCCAG |
| | | CGCTT | CGCUU |
| | | CGCAC | CGCAC |
| | Ex2 STOP | GAGCT | GAGCU |
| | (pos 7) | GCAGC | GCAGC |
| | | TGAAT | UGAAU |
| | | CACAC | CACAC |
| | Ex3 STOP | CTGGC | CUGGC |
| | (pos 5) | AGGGG | AGGGG |
| | | GGCCC | GGCCC |
| | | AGCAC | AGCAC |
| | Ex3 STOP | CTCCC | CUCCC |
| | (pos 5) | AGCGC | AGCGC |
| | | CTGAC | CUGAC |
| | | GCCCC | GCCCC |
| | Ex3 STOP | CCCCC | CCCCC |
| | (pos 8) | TGCCA | UGCCA |
| | | GTATA | GUAUA |
| | | GCCTG | GCCUG |
| | Ex3 STOP | CCCCC | CCCCC |
| | (pos 9) | CTGCC | CUGCC |
| | | AGTAT | AGUAU |
| | | AGCCT | AGCCU |
| CD82 | Exon 1 ATG | TGAGC | UGAGC |
| | | CCATC | CCAUC |
| | | CCGCC | CCGCC |
| | | AGTCC | AGUCC |
| | Exon 3 SD | TCACC | UCACC |
| | | AGCCC | AGCCC |
| | | CAGCA | CAGCA |
| | | GGCAG | GGCAG |
| | Exon 4 SA | AAGTA | AAGUA |
| | | CTGGG | CUGGG |
| | | GACAC | GACAC |
| | | AGAGC | AGAGC |
| | Exon 6 SA 1 | ACTTC | ACUUC |
| | | ACCTG | ACCUG |
| | | GGCAA | GGCAA |
| | | GGCAG | GGCAG |
| | Exon 6 SA 2 | CACTT | CACUU |
| | | CACCT | CACCU |
| | | GGGCA | GGGCA |
| | | AGGCA | AGGCA |
| | Exon 6 SD | CCGCA | CCGCA |
| | | CACCT | CACCU |
| | | CCTGG | CCUGG |
| | | TACAC | UACAC |
| | Exon 7 SA | AGCCC | AGCCC |
| | | TGCAA | UGCAA |
| | | GGGCA | GGGCA |
| | | GAATG | GAAUG |
| | Exon 8 SA | CCAGG | CCAGG |
| | | AGCTG | AGCUG |
| | | TGGGG | UGGGG |
| | | AGAGG | AGAGG |
| CD86 | Ex2 SD | GTTCT | GUUCU |
| | (pos 8) | TACCA | UACCA |
| | | GAGAG | GAGAG |
| | | CAGGA | CAGGA |
| | Ex3 SA | GCACC | GCACC |
| | (pos 5) | TAAAA | UAAAA |
| | | AAGAA | AAGAA |
| | | GGTTA | GGUUA |
| | Ex3 STOP | TTGGC | UUGGC |
| | (pos 5) | AGGAC | AGGAC |
| | | CAGGA | CAGGA |
| | | AAACT | AAACU |
| | Ex3 STOP | CAATC | CAAUC |
| | (pos 8) | TTCAG | UUCAG |
| | | ATCAA | AUCAA |
| | | GGACA | GGACA |
| | Ex5 STOP | GTAAT | GUAAU |
| | (pos 6/7) | CCAAG | CCAAG |
| | | GAATG | GAAUG |
| | | TGGTC | UGGUC |
| | Ex6 STOP | AGAGT | AGAGU |
| | (pos 9) | GAACA | GAACA |
| | | GACCA | GACCA |
| | | AGAAA | AGAAA |
| CD160 | Exon 1 STOP | GGACA | GGACA |
| | | TCCAG | UCCAG |
| | | TCTGG | UCUGG |
| | | TGGTG | UGGUG |
| | Exon 2 SA | AATGC | AAUGC |
| | | ATCCT | AUCCU |
| | | GGAAT | GGAAU |
| | | GGAAA | GGAAA |
| | Exon 2 SD | GCACT | GCACU |
| | | CACCT | CACCU |
| | | GTGAA | GUGAA |
| | | TAGAA | UAGAA |
| | Exon 2 STOP | TAAAA | UAAAA |
| | | CAGCT | CAGCU |
| | | GAGAC | GAGAC |
| | | TTAAA | UUAAA |
| | Exon 3 STOP 1 | GCTTC | GCUUC |
| | | CTACA | CUACA |
| | | AGAAA | AGAAA |
| | | AGGTC | AGGUC |
| | Exon 3 STOP 2 | TTACC | UUACC |
| | | CAGAC | CAGAC |
| | | CTTTT | CUUUU |
| | | CTTGT | CUUGU |
| CD244 | Exon 1 ATG 1 | CAGCA | CAGCA |
| | | TTTCC | UUUCC |
| | | ACAGG | ACAGG |
| | | ACAGA | ACAGA |
| | Exon 1 ATG 2 | CCAGC | CCAGC |
| | | ATTTC | AUUUC |
| | | CACAG | CACAG |
| | | GACAG | GACAG |
| | Exon 2 SD | ACTTA | ACUUA |
| | | CCAAA | CCAAA |
| | | TACAA | UACAA |
| | | AAACC | AAACC |
| | Exon 3 SA | GATTC | GAUUC |
| | | TGATC | UGAUC |
| | | AGAAA | AGAAA |
| | | GGCAT | GGCAU |
| | Exon 4 SA | TGAAT | UGAAU |
| | | TCTGA | UCUGA |
| | | GGAAT | GGAAU |
| | | ACAGA | ACAGA |
| | Exon 5 SA | ATGAC | AUGAC |
| | | ATACG | AUACG |
| | | TGATT | UGAUU |
| | | TCTCC | UCUCC |
| | Exon 6 SA | TCCTG | UCCUG |
| | | CTCCT | CUCCU |
| | | GCACA | GCACA |
| | | AGAAA | AGAAA |
| | Exon 8 SA | TCACC | UCACC |
| | | CTAGG | CUAGG |
| | | AGCAA | AGCAA |
| | | AACAA | AACAA |
| CD276 | Exon 1 ATG | CGCAG | CGCAG |
| | | CATCT | CAUCU |
| | | TCCTG | UCCUG |
| | | TGAGG | UGAGG |
| | Exon 2 SA | GCTCC | GCUCC |
| | | TGGGG | UGGGG |
| | | GTAGG | GUAGG |
| | | GGGAG | GGGAG |
| | Exon 2 SD | GGTGC | GGUGC |
| | | TCACC | UCACC |
| | | GGCCA | GGCCA |
| | | CCTGC | CCUGC |
| | Exon 3 SA 1 | AGGGA | AGGGA |
| | | GCTGG | GCUGG |
| | | AGGTG | AGGUG |
| | | ACAGA | ACAGA |
| | Exon 3 SA 2 | TAGGG | UAGGG |
| | | AGCTG | AGCUG |
| | | GAGGT | GAGGU |
| | | GACAG | GACAG |
| | Exon 3 SD 1 | GCAAC | GCAAC |
| | | CTGTG | CUGUG |
| | | GGGCT | GGGCU |
| | | TCTCT | UCUCU |
| | Exon 3 SD 2 | AGCAA | AGCAA |
| | | CCTGT | CCUGU |
| | | GGGGC | GGGGC |
| | | TTCTC | UUCUC |
| | Exon 4 SA 1 | CTCCT | CUCCU |
| | | GGGGG | GGGGG |
| | | CGGGG | CGGGG |
| | | TCAGA | UCAGA |
| | Exon 4 SA 2 | GCTCC | GCUCC |
| | | TGGGG | UGGGG |
| | | GCGGG | GCGGG |
| | | GTCAG | GUCAG |
| | Exon 4 SD | GGTGC | GGUGC |
| | | TCACC | UCACC |
| | | GGCCA | GGCCA |
| | | CCTGC | CCUGC |
| | Exon 5 SA 1 | AGGGA | AGGGA |
| | | GCTGG | GCUGG |
| | | AGGTG | AGGUG |
| | | ACAGA | ACAGA |
| | Exon 5 SA 2 | TAGGG | UAGGG |
| | | AGCTG | AGCUG |
| | | GAGGT | GAGGU |
| | | GACAG | GACAG |
| | Exon 8 SA | GCAGG | GCAGG |
| | | GCTGT | GCUGU |
| | | AAAAA | AAAAA |
| | | AAGGA | AAGGA |
| | Exon 9 SA 1 | CATCA | CAUCA |
| | | TCTTC | UCUUC |
| | | ATTTC | AUUUC |
| | | ATGAT | AUGAU |
| | Exon 9 SA 2 | CCATC | CCAUC |
| | | ATCTT | AUCUU |
| | | CATTT | CAUUU |
| | | CATGA | CAUGA |
| CDK8 | Exon 1 ATG | GTCCA | GUCCA |
| | | TTGTC | UUGUC |
| | | ACAGC | ACAGC |
| | | CTCTG | CUCUG |
| | Exon 1 SD | CACTC | CACUC |
| | | ACCCA | ACCCA |
| | | TCTTT | UCUUU |
| | | CCTCT | CCUCU |
| | Exon 10 SD 2 | ACTTA | ACUUA |
| | | CTCTG | CUCUG |
| | | ATGTA | AUGUA |
| | | GGAAG | GGAAG |
| | Exon 10 SD 1 | CTTAC | CUUAC |
| | | TCTGA | UCUGA |
| | | TGTAG | UGUAG |
| | | GAAGT | GAAGU |
| | Exon 12 SD | TTGGA | UUGGA |
| | | ATACC | AUACC |
| | | TGATA | UGAUA |
| | | GTCTG | GUCUG |
| | Exon 13 SA 2 | GGAAC | GGAAC |
| | | GCTGG | GCUGG |
| | | AAAGG | AAAGG |
| | | AGATG | AGAUG |
| | Exon 13 SA 1 | GAACG | GAACG |
| | | CTGGA | CUGGA |
| | | AAGGA | AAGGA |
| | | GATGA | GAUGA |
| CDKN1B | Exon 1 ATG | ACGTT | ACGUU |
| | | TGACA | UGACA |
| | | TCTTT | UCUUU |
| | | CTCCC | CUCCC |
| | Exon 1 STOP 1 | CAAAC | CAAAC |
| | | GTGCG | GUGCG |
| | | AGTGT | AGUGU |
| | | CTAAC | CUAAC |
| | Exon 1 STOP 2 | CGAGT | CGAGU |
| | | GGCAA | GGCAA |
| | | GAGGT | GAGGU |
| | | GGAGA | GGAGA |
| | Exon 1 STOP 3 | GAGTG | GAGUG |
| | | GCAAG | GCAAG |
| | | AGGTG | AGGUG |
| | | GAGAA | GAGAA |
| | Exon 1 STOP 4 | AGGAG | AGGAG |
| | | AGCCA | AGCCA |
| | | GGATG | GGAUG |
| | | TCAGC | UCAGC |
| | Exon 1 STOP 5 | GGACA | GGACA |
| | | GCCAG | GCCAG |
| | | ACGGG | ACGGG |
| | | GTTAG | GUUAG |
| | Exon 1 STOP 6 | CGGAG | CGGAG |
| | | CAATG | CAAUG |
| | | CGCAG | CGCAG |
| | | GAATA | GAAUA |
| | Exon 1 STOP 7 | AGGAA | AGGAA |
| | | GCGAC | GCGAC |
| | | CTGCA | CUGCA |
| | | ACCGA | ACCGA |
| | Exon 2 STOP | GAGCA | GAGCA |
| | | GACGC | GACGC |
| | | CCAAG | CCAAG |
| | | AAGCC | AAGCC |
| CSF2 | Exon 1 STOP 1 | GCTGC | GCUGC |
| | | AGAGC | AGAGC |
| | | CTGCT | CUGCU |
| | | GCTCT | GCUCU |
| | Exon 1 STOP 2 | GCTCC | GCUCC |
| | | CAGGG | CAGGG |
| | | CTGCG | CUGCG |
| | | TGCTG | UGCUG |
| | Exon 1 STOP 3 | TGCTC | UGCUC |
| | | CCAGG | CCAGG |
| | | GCTGC | GCUGC |
| | | GTGCT | GUGCU |
| | Exon 1 STOP 4 | ATGCT | AUGCU |
| | | CCCAG | CCCAG |
| | | GGCTG | GGCUG |
| | | CGTGC | CGUGC |
| | Exon 3 SD | AGGCA | AGGCA |
| | (pos 10) | CTCAC | CUCAC |
| | | CGGGG | CGGGG |
| | | TTGGA | UUGGA |
| | Exon 4 STOP 1 | CTGGC | CUGGC |
| | | TCCCA | UCCCA |
| | | GCAGT | GCAGU |
| | | CAAAG | CAAAG |
| CSK | Exon 1 ATG | TGACA | UGACA |
| | | TCTTC | UCUUC |
| | | TCAGG | UCAGG |
| | | AGCTC | AGCUC |
| | Exon 3 SD | TCACG | UCACG |
| | | GCATG | GCAUG |
| | | AGGCT | AGGCU |
| | | GAGTT | GAGUU |
| | Exon 4 SA 1 | GAACC | GAACC |
| | | AACTG | AACUG |
| | | GGGAG | GGGAG |
| | | CAGCA | CAGCA |
| | Exon 4 SA 2 | GGAAC | GGAAC |
| | | CAACT | CAACU |
| | | GGGGA | GGGGA |
| | | GCAGC | GCAGC |
| | Exon 4 SD | TCACC | UCACC |
| | | TCCAC | UCCAC |
| | | CAGCT | CAGCU |
| | | GCATG | GCAUG |
| | Exon 5 SA 1 | GTAGT | GUAGU |
| | | GCTGC | GCUGC |
| | | AGGGT | AGGGU |
| | | GTGGG | GUGGG |
| | Exon 5 SA 2 | TGTAG | UGUAG |
| | | TGCTG | UGCUG |
| | | CAGGG | CAGGG |
| | | TGTGG | UGUGG |
| | Exon 7 SA 1 | CACGT | CACGU |
| | | CTGGG | CUGGG |
| | | GGCAG | GGCAG |
| | | AGAGG | AGAGG |
| | Exon 7 SA 2 | CATCA | CAUCA |
| | | CGTCT | CGUCU |
| | | GGGGG | GGGGG |
| | | CAGAG | CAGAG |
| | Exon 9 SA | AGGCT | AGGCU |
| | | CCCCT | CCCCU |
| | | GGGGG | GGGGG |
| | | CAGGA | CAGGA |
| | Exon 9 SD | TCACT | UCACU |
| | | CACAG | CACAG |
| | | CGAGA | CGAGA |
| | | ACTTG | ACUUG |
| | Exon 10 SD | CAGCC | CAGCC |
| | | CCACC | CCACC |
| | | TTCTC | UUCUC |
| | | TCTCA | UCUCA |
| | Exon 11 SA | GAGAA | GAGAA |
| | | TTTCT | UUUCU |
| | | GCCAT | GCCAU |
| | | GTGGA | GUGGA |
| | Exon USD | ATACT | AUACU |
| | | CACAA | CACAA |
| | | TTCTT | UUCUU |
| | | GGATA | GGAUA |
| | Exon 12 SA | CAGGG | CAGGG |
| | | GCTGT | GCUGU |
| | | GGCCA | GGCCA |
| | | GGGGG | GGGGG |
| CTLA-4 | Exon 1 SD | ACTCA | ACUCA |
| | (pos 6) | CCTTT | CCUUU |
| | | GCAGA | GCAGA |
| | | AGACA | AGACA |
| | Exon 1 SD | CACTC | CACUC |
| | | ACCTT | ACCUU |
| | | TGCAG | UGCAG |
| | | AAGAC | AAGAC |
| | Exon 1 STOP | AGGGC | AGGGC |
| | (pos5) | CAGGT | CAGGU |
| | | CCTGG | CCUGG |
| | | TAGCC | UAGCC |
| | Exon 2 STOP | GGCCC | GGCCC |
| | | AGCCT | AGCCU |
| | | GCTGT | GCUGU |
| | | GGTAC | GGUAC |
| | Exon 2 STOP | GCTTC | GCUUC |
| | (pos 8) | GGCAG | GGCAG |
| | | GCTGA | GCUGA |
| | | CAGCC | CAGCC |
| | Exon 2 STOP | ++TATCC | ++UAUCC |
| | | AAGGA | AAGGA |
| | | CTGAG | CUGAG |
| | | GGCCA | GGCCA |
| | Exon 2 STOP | GGAAC | GGAAC |
| | | CCAGA | CCAGA |
| | | TTTAT | UUUAU |
| | | GTAAT | GUAAU |
| | Exon 2 SD | GCTCA | GCUCA |
| | | CCAAT | CCAAU |
| | | TACAT | UACAU |
| | | AAATC | AAAUC |
| | Exon 2 SD | CTCAC | CUCAC |
| | | CAATT | CAAUU |
| | | ACATA | ACAUA |
| | | AATCT | AAUCU |
| | Exon 1 STOP | CTCAG | CUCAG |
| | | CTGAA | CUGAA |
| | | CCTGG | CCUGG |
| | | CTACC | CUACC |
| CUL3 | Exon 1/2 | TTAAC | UUAAC |
| | SA ATG | ATCTA | AUCUA |
| | | CTACA | CUACA |
| | | TACAA | UACAA |
| | Exon 6 SD | CTTAC | CUUAC |
| | | CTGGA | CUGGA |
| | | TATAG | UAUAG |
| | | TCAAC | UCAAC |
| | Exon 2 STOP | ATCCA | AUCCA |
| | | GCGTA | GCGUA |
| | | AGAAT | AGAAU |
| | | AACAG | AACAG |
| | Exon 4 STOP 1 | GTATG | GUAUG |
| | | TACAA | UACAA |
| | | CAAAA | CAAAA |
| | | TAATG | UAAUG |
| | Exon 4 STOP 2 | CGAGA | CGAGA |
| | | TCAAG | UCAAG |
| | | TTGTA | UUGUA |
| | | CGTTA | CGUUA |
| | Exon 5 STOP | TGCCA | UGCCA |
| | | GATGT | GAUGU |
| | | TAATG | UAAUG |
| | | A1TTT | AUUUU |
| | Exon 9 STOP | ATGTC | AUGUC |
| | | AGTTC | AGUUC |
| | | ACGTC | ACGUC |
| | | AAAAC | AAAAC |
| | Exon 14 STOP | GCCCT | GCCCU |
| | | ACAGT | ACAGU |
| | | CCCTC | CCCUC |
| | | GCCTG | GCCUG |
| Cyp11a1 | Exon 1 STOP 1 | GTCCA | GUCCA |
| | (pos 4) | GAATT | GAAUU |
| | | TCCAG | UCCAG |
| | | AAGTA | AAGUA |
| | Exon 2 SA 1 | TCCCT | UCCCU |
| | (pos 4) | GGAGG | GGAGG |
| | | GGTGG | GGUGG |
| | | GGGAG | GGGAG |
| | Exon 2 SD 1 | TCACT | UCACU |
| | (pos 4) | TCAAC | UCAAC |
| | | AGGAC | AGGAC |
| | | TCCTA | UCCUA |
| | Exon 3 SD 1 | CCTTA | CCUUA |
| | (pos 6) | CACTC | CACUC |
| | | AAAGG | AAAGG |
| | | CAAAG | CAAAG |
| | Exon 4 SA | ATGGC | AUGGC |
| | (pos 5) | TGCAG | UGCAG |
| | | GGAGA | GGAGA |
| | | GGAAG | GGAAG |
| | Exon 4 STOP 1 | GGAGC | GGAGC |
| | (pos 8) | GCCAG | GCCAG |
| | | GGGAT | GGGAU |
| | | GCTGG | GCUGG |
| | Exon 4 STOP 2 | TCACG | UCACG |
| | (pos 8) | TCCCA | UCCCA |
| | | TGCAG | UGCAG |
| | | CCACA | CCACA |
| | Exon 6 SA | TGGAC | UGGAC |
| | (pos 8) | GTCTG | GUCUG |
| | | GTGGG | GUGGG |
| | | GAGTA | GAGUA |
| | Exon 8 STOP 1 | ACTCA | ACUCA |
| | (pos 6) | CATTG | CAUUG |
| | | ATGAG | AUGAG |
| | | GAAGA | GAAGA |
| | Exon 9 SA | CAGCA | CAGCA |
| | (pos 7) | TCTGA | UCUGA |
| | | GAAAG | GAAAG |
| | | GCAGA | GCAGA |
| | Exon 9 STOP 1 | AATCC | AAUCC |
| | (pos 5) | AACAC | AACAC |
| | | CTCAG | CUCAG |
| | | CGATG | CGAUG |
| | Exon 9 STOP 2 | ATCCA | AUCCA |
| | (pos 4) | ACACC | ACACC |
| | | TCAGC | UCAGC |
| | | GATGT | GAUGU |
| DCK | Exon 1 ATG | GTGGC | GUGGC |
| | | CATTC | CAUUC |
| | | CTTAG | CUUAG |
| | | TCTTG | UCUUG |
| | Exon 1 SD | CTTAC | CUUAC |
| | | CGATG | CGAUG |
| | | TTCCC | UUCCC |
| | | TTCGA | UUCGA |
| | Exon 2 STOP 1 | TTAAA | UUAAA |
| | | CAATT | CAAUU |
| | | GTGTG | GUGUG |
| | | AAGAT | AAGAU |
| | Exon 2 STOP 2 | TAAAC | UAAAC |
| | | AATTG | AAUUG |
| | | TGTGA | UGUGA |
| | | AGATT | AGAUU |
| | Exon 2 STOP 3 | CACCA | CACCA |
| | | TCTGG | UCUGG |
| | | CAACA | CAACA |
| | | GGTTC | GGUUC |
| | Exon 2 STOP 4 | AAGTA | AAGUA |
| | | CTCAA | CUCAA |
| | | GATGA | GAUGA |
| | | ATTTG | AUUUG |
| | Exon 3 STOP 1 | CAATG | CAAUG |
| | | TCTCA | UCUCA |
| | | GAAAA | GAAAA |
| | | ATGGT | AUGGU |
| | Exon 3 STOP 2 | GCTCA | GCUCA |
| | | GCTTG | GCUUG |
| | | CCTCT | CCUCU |
| | | CTGAA | CUGAA |
| | Exon 4 STOP 1 | TTTAT | UUUAU |
| | | CAAGA | CAAGA |
| | | CTGGC | CUGGC |
| | | ATGAC | AUGAC |
| | Exon 4 STOP 2 | ATTTG | AUUUG |
| | | GCCAA | GCCAA |
| | | AGCCT | AGCCU |
| | | TGAAT | UGAAU |
| | Exon 4 STOP 3 | TTATC | UUAUC |
| | | TTCAA | UUCAA |
| | | GCCAC | GCCAC |
| | | TCCAG | UCCAG |
| | Exon 5 SD | CTTAC | CUUAC |
| | | TTCAG | UUCAG |
| | | TGTCC | UGUCC |
| | | TATGC | UAUGC |
| DGKA | Exon 1 ATG | ACCCC | ACCCC |
| | | ATTTT | AUUUU |
| | | GTTCC | GUUCC |
| | | GCCTC | GCCUC |
| | Exon 5 SD | TCACA | UCACA |
| | | TTCTA | UUCUA |
| | | ACTTG | ACUUG |
| | | TCTTC | UCUUC |
| | Exon 6 SA 1 | TGACT | UGACU |
| | | GTGGG | GUGGG |
| | | GTGTT | GUGUU |
| | | T1AGG | UUAGG |
| | Exon 6 SA 2 | GTGAC | GUGAC |
| | | TGTGG | UGUGG |
| | | GGTGT | GGUGU |
| | | TTTAG | UUUAG |
| | Exon 6 SA 3 | GGTGA | GGUGA |
| | | CTGTG | CUGUG |
| | | GGGTG | GGGUG |
| | | TTTTA | UUUUA |
| | Exon 6 SA 4 | AGG1G | AGGUG |
| | | AC1G1 | ACUGU |
| | | GGGG1 | GGGGU |
| | | GTT1T | GUUUU |
| | Exon 7 SA 1 | AATCT | AAUCU |
| | | GAGCA | GAGCA |
| | | CAGAG | CAGAG |
| | | TGGAA | UGGAA |
| | Exon 7 SA 2 | TGAAG | UGAAG |
| | | AATCT | AAUCU |
| | | GAGCA | GAGCA |
| | | CAGAG | CAGAG |
| | Exon 10 SA | TGATT | UGAUU |
| | | GGACC | GGACC |
| | | TTGGG | UUGGG |
| | | GAGAA | GAGAA |
| | Exon USA 1 | GTGGA | GUGGA |
| | | TCTGA | UCUGA |
| | | AAGAC | AAGAC |
| | | GAGGT | GAGGU |
| | Exon 11 SA 2 | CGTGG | CGUGG |
| | | ATCTG | AUCUG |
| | | AAAGA | AAAGA |
| | | CGAGG | CGAGG |
| | Exon 12 SD | CTGTA | CUGUA |
| | | CCCGC | CCCGC |
| | | AGAGC | AGAGC |
| | | CTCAG | CUCAG |
| | Exon 15 SA | AATCG | AAUCG |
| | | GAGCC | GAGCC |
| | | TGAGA | UGAGA |
| | | CAAAG | CAAAG |
| | Exon 16 SD | ACTTA | ACUUA |
| | | CCTCC | CCUCC |
| | | TCCCC | UCCCC |
| | | ATCTT | AUCUU |
| | Exon 17 SA | AACCT | AACCU |
| | | AGGAG | AGGAG |
| | | TGGAG | UGGAG |
| | | AAGAC | AAGAC |
| | Exon 18 SA | AGAGG | AGAGG |
| | | CATCC | CAUCC |
| | | TGGAG | UGGAG |
| | | AGTTC | AGUUC |
| | Exon 20 SA | CCCAC | CCCAC |
| | | AGATC | AGAUC |
| | | TGAGA | UGAGA |
| | | GGAGG | GGAGG |
| | Exon 21 SA 1 | TTAGG | UUAGG |
| | | TCTGG | UCUGG |
| | | GGACG | GGACG |
| | | AAGTA | AAGUA |
| | Exon 21 SA 2 | CTTAG | CUUAG |
| | | GTCTG | GUCUG |
| | | GGGAC | GGGAC |
| | | GAAGT | GAAGU |
| | Exon 22 SA | TGTGG | UGUGG |
| | | TGCTA | UGCUA |
| | | TAGGA | UAGGA |
| | | GGCCA | GGCCA |
| | Iso SA | GACCC | GACCC |
| | | TGGAA | UGGAA |
| | | GAGTT | GAGUU |
| | | GGGGC | GGGGC |
| DGKZ | Exon 3 SA | CTGAC | CUGAC |
| | | TCCTA | UCCUA |
| | | GCCAC | GCCAC |
| | | GGGAT | GGGAU |
| | Exon 4 SA | CTGCT | CUGCU |
| | | GCAGG | GCAGG |
| | | ACAGG | ACAGG |
| | | AAGAG | AAGAG |
| | Exon 5 SD | ACTTA | ACUUA |
| | | CCTCG | CCUCG |
| | | CGGAC | CGGAC |
| | | ATTCC | AUUCC |
| | Exon 6 SA 1 | AAGGT | AAGGU |
| | | TGGCT | UGGCU |
| | | GGGGG | GGGGG |
| | | AGAAG | AGAAG |
| | Exon 6 SA 2 | AAAGG | AAAGG |
| | | TTGGC | UUGGC |
| | | TGGGG | UGGGG |
| | | GAGAA | GAGAA |
| | Exon 7 SA 1 | ATCCC | AUCCC |
| | | TGAGG | UGAGG |
| | | GTAGA | GUAGA |
| | | CAGGA | CAGGA |
| | Exon 7 SA 2 | TGGAA | UGGAA |
| | | TCCCT | UCCCU |
| | | GAGGG | GAGGG |
| | | TAGAC | UAGAC |
| | Exon 8 SA 1 | GTGGT | GUGGU |
| | | ACTGA | ACUGA |
| | | GGAGA | GGAGA |
| | | GCGAG | GCGAG |
| | Exon 8 SA 2 | TGTGG | UGUGG |
| | | TACTG | UACUG |
| | | AGGAG | AGGAG |
| | | AGCGA | AGCGA |
| | Exon 8 SA 3 | CTGTG | CUGUG |
| | | GTACT | GUACU |
| | | GAGGA | GAGGA |
| | | GAGCG | GAGCG |
| | Exon 8 SD 1 | AGTAC | AGUAC |
| | | TCACC | UCACC |
| | | TGGGG | UGGGG |
| | | CCTCC | CCUCC |
| | Exon 9 SA 1 | GTATT | GUAUU |
| | | CTGCA | CUGCA |
| | | AGGGA | AGGGA |
| | | AGCAG | AGCAG |
| | Exon 9 SA 2 | AGTAT | AGUAU |
| | | TCTGC | UCUGC |
| | | AAGGG | AAGGG |
| | | AAGCA | AAGCA |
| | Exon 9 SA 3 | GAGTA | GAGUA |
| | | TTCTG | UUCUG |
| | | CAAGG | CAAGG |
| | | GAAGC | GAAGC |
| | Exon 10 SA | CTCCT | CUCCU |
| | | GGGAG | GGGAG |
| | | ACAAG | ACAAG |
| | | GTGAG | GUGAG |
| | Exon 11 SA | TTTGC | UUUGC |
| | | ACCCT | ACCCU |
| | | GGATG | GGAUG |
| | | GGATG | GGAUG |
| | Exon 12 SA | AGCCT | AGCCU |
| | | GGGCT | GGGCU |
| | | CATGG | CAUGG |
| | | GAAGA | GAAGA |
| | Exon 13 SD 1 | GCTTA | GCUUA |
| | | CCCCA | CCCCA |
| | | CCCCA | CCCCA |
| | | GTTGA | GUUGA |
| | Exon 13 SD 2 | TGCTT | UGCUU |
| | | ACCCC | ACCCC |
| | | ACCCC | ACCCC |
| | | AGTTG | AGUUG |
| | Exon 14 SA 1 | TAGCC | UAGCC |
| | | CTGGG | CUGGG |
| | | GGAGC | GGAGC |
| | | AGGCA | AGGCA |
| | Exon 14 SA 2 | GTAGC | GUAGC |
| | | CCTGG | CCUGG |
| | | GGGAG | GGGAG |
| | | CAGGC | CAGGC |
| | Exon 15 SA 1 | GGCAA | GGCAA |
| | | CTGGA | CUGGA |
| | | AAAAG | AAAAG |
| | | GTCAA | GUCAA |
| | Exon 15 SA 2 | GGGCA | GGGCA |
| | | ACTGG | ACUGG |
| | | AAAAA | AAAAA |
| | | GGTCA | GGUCA |
| | Exon 15 SD | GCTGC | GCUGC |
| | | CAACC | CAACC |
| | | TCGAG | UCGAG |
| | | ACTCG | ACUCG |
| | Exon 17 SA 1 | AGCTG | AGCUG |
| | | TCTGT | UCUGU |
| | | GGGAG | GGGAG |
| | | ACAGA | ACAGA |
| | Exon 17 SA 2 | AAGCT | AAGCU |
| | | GTCTG | GUCUG |
| | | TGGGA | UGGGA |
| | | GACAG | GACAG |
| | Exon 17 SD | CTCCT | CUCCU |
| | | CACCT | CACCU |
| | | GGGGA | GGGGA |
| | | TGTTC | UGUUC |
| | Exon 18 SD | CCCAC | CCCAC |
| | | TCACC | UCACC |
| | | AACGA | AACGA |
| | | CGTCA | CGUCA |
| | Exon 19 SD 1 | GTACT | GUACU |
| | | CGCTG | CGCUG |
| | | TGCAG | UGCAG |
| | | GGGGG | GGGGG |
| | Exon 19 SD 2 | GACGT | GACGU |
| | | ACTCG | ACUCG |
| | | CTGTG | CUGUG |
| | | CAGGG | CAGGG |
| | Exon 19 SD 3 | GGACG | GGACG |
| | | TACTC | UACUC |
| | | GCTGT | GCUGU |
| | | GCAGG | GCAGG |
| | Exon 19 SD 4 | GGGAC | GGGAC |
| | | GTACT | GUACU |
| | | CGCTG | CGCUG |
| | | TGCAG | UGCAG |
| | Exon 20 SA | TGCTG | UGCUG |
| | | GCTGT | GCUGU |
| | | CGGGC | CGGGC |
| | | AGGGC | AGGGC |
| | Exon 22 SA | GCTCC | GCUCC |
| | | TGTTG | UGUUG |
| | | AGAGA | AGAGA |
| | | AGCAA | AGCAA |
| | Exon 23 SA | AGTGG | AGUGG |
| | | TGGCT | UGGCU |
| | | GTGGG | GUGGG |
| | | AGAGA | AGAGA |
| | Exon 24 SA 1 | GCTCC | GCUCC |
| | | TGTGG | UGUGG |
| | | GGGGA | GGGGA |
| | | GGTCA | GGUCA |
| | Exon 24 SA 2 | TGCTC | UGCUC |
| | | CTGTG | CUGUG |
| | | GGGGG | GGGGG |
| | | AGGTC | AGGUC |
| | Exon 24 SD | TCACC | UCACC |
| | | GGGGC | GGGGC |
| | | GTGGG | GUGGG |
| | | TGAGC | UGAGC |
| | Exon 27 SA | GGAGC | GGAGC |
| | | TGAGG | UGAGG |
| | | GCAGG | GCAGG |
| | | AGGCA | AGGCA |
| | Exon 27 SD | CCGGC | CCGGC |
| | | TCACC | UCACC |
| | | GTGGT | GUGGU |
| | | CCAGC | CCAGC |
| | Exon 28 SA 1 | GGGGG | GGGGG |
| | | CTGGA | CUGGA |
| | | GAAGG | GAAGG |
| | | GGAGG | GGAGG |
| | Exon 28 SA 2 | TGGGG | UGGGG |
| | | GGGCT | GGGCU |
| | | GGAGA | GGAGA |
| | | AGGGG | AGGGG |
| | Exon 29 SA 1 | CCCGC | CCCGC |
| | | TGGGG | UGGGG |
| | | GAGCA | GAGCA |
| | | GGGGC | GGGGC |
| | Exon 29 SA 2 | TCTCC | UCUCC |
| | | CCGCT | CCGCU |
| | | GGGGG | GGGGG |
| | | AGCAG | AGCAG |
| | iso exon 18 SA 1 | ACACT | ACACU |
| | | GGAGG | GGAGG |
| | | CGGGC | CGGGC |
| | | GGGGA | GGGGA |
| | iso exon 18 SA 2 | CACAC | CACAC |
| | | TGGAG | UGGAG |
| | | GCGGG | GCGGG |
| | | CGGGG | CGGGG |
| | iso exon 18 SA 3 | CATCA | CAUCA |
| | | CACTG | CACUG |
| | | GAGGC | GAGGC |
| | | GGGCG | GGGCG |
| | Isoform exon | CTACG | CUACG |
| | 1/2 SD | CGAGC | CGAGC |
| | | AGGGC | AGGGC |
| | | GCTCC | GCUCC |
| | Isoform | TGGCT | UGGCU |
| | exon 2 SA | GTTGG | GUUGG |
| | | GCACA | GCACA |
| | | GAGAC | GAGAC |
| | Isoform | ACTGA | ACUGA |
| | exon 4 SA | CTTCT | CUUCU |
| | | GCTGC | GCUGC |
| | | AGGAC | AGGAC |
| DHX37 | Exon 1 ATG 1 | TCCCC | UCCCC |
| | | ATGGC | AUGGC |
| | | GACTA | GACUA |
| | | GGCCA | GGCCA |
| | Exon 1 ATG 2 | TTCCC | UUCCC |
| | | CATGG | CAUGG |
| | | CGACT | CGACU |
| | | AGGCC | AGGCC |
| | Exon 4 SD 1 | GAACC | GAACC |
| | | TGCAT | UGCAU |
| | | TTCCG | UUCCG |
| | | GGGAG | GGGAG |
| | Exon 4 SD 2 | GTGGC | GUGGC |
| | | GAACC | GAACC |
| | | TGCAT | UGCAU |
| | | TTCCG | UUCCG |
| | Exon 5 SA | TCACT | UCACU |
| | | GGGGG | GGGGG |
| | | AGGAA | AGGAA |
| | | GAACA | GAACA |
| | Exon 7 SA 1 | ACCCT | ACCCU |
| | | GTATG | GUAUG |
| | | GGCAG | GGCAG |
| | | AGTTC | AGUUC |
| | Exon 7 SA 2 | GACCC | GACCC |
| | | TGTAT | UGUAU |
| | | GGGCA | GGGCA |
| | | GAGTT | GAGUU |
| | Exon 8 SA 1 | AAGTC | AAGUC |
| | | CTGGG | CUGGG |
| | | GGGAG | GGGAG |
| | | GTCCG | GUCCG |
| | Exon 8 SA 2 | GAAGT | GAAGU |
| | | CCTGG | CCUGG |
| | | GGGGA | GGGGA |
| | | GGTCC | GGUCC |
| | Exon 8 SA 3 | GGAAG | GGAAG |
| | | TCCTG | UCCUG |
| | | GGGGG | GGGGG |
| | | AGGTC | AGGUC |
| | Exon 9 SA | AGGTT | AGGUU |
| | | CCTCT | CCUCU |
| | | GCAAA | GCAAA |
| | | AGGAC | AGGAC |
| | Exon 9 SD 1 | GTTAC | GUUAC |
| | | CTTGA | CUUGA |
| | | TGACC | UGACC |
| | | GGCGG | GGCGG |
| | Exon 9 SD 2 | TGTGT | UGUGU |
| | | TACCT | UACCU |
| | | TGATG | UGAUG |
| | | ACCGG | ACCGG |
| | Exon 10 SA 1 | CACCT | CACCU |
| | | GTGGG | GUGGG |
| | | ACGCC | ACGCC |
| | | CAGGA | CAGGA |
| | Exon 10 SA 2 | ATTCC | AUUCC |
| | | ACCTG | ACCUG |
| | | TGGGA | UGGGA |
| | | CGCCC | CGCCC |
| | Exon 10 SD | CCTCA | CCUCA |
| | | CCTGC | CCUGC |
| | | GGGCA | GGGCA |
| | | GCATC | GCAUC |
| | Exon USA 1 | ATGCC | AUGCC |
| | | ACCTG | ACCUG |
| | | TGGAA | UGGAA |
| | | AGAAT | AGAAU |
| | Exon 11 SA 2 | GATGC | GAUGC |
| | | CACCT | CACCU |
| | | GTGGA | GUGGA |
| | | AAGAA | AAGAA |
| | Exon 11 SD | TTTAC | UUUAC |
| | | CTTGT | CUUGU |
| | | GGCCG | GGCCG |
| | | GGCTC | GGCUC |
| | Exon 12 SA 1 | TTTCT | UUUCU |
| | | GGGAG | GGGAG |
| | | AGGGG | AGGGG |
| | | CAGGT | CAGGU |
| | Exon 12 SA 2 | TCCTT | UCCUU |
| | | TTCTG | UUCUG |
| | | GGAGA | GGAGA |
| | | GGGGC | GGGGC |
| | Exon 14 SA | CTCAC | CUCAC |
| | | CTGGA | CUGGA |
| | | GGGAA | GGGAA |
| | | AGCAG | AGCAG |
| | Exon 14 SD 1 | TTACC | UUACC |
| | | TGTGC | UGUGC |
| | | TTGCT | UUGCU |
| | | TCTCT | UCUCU |
| | Exon 14 SD 2 | GTTAC | GUUAC |
| | | CTGTG | CUGUG |
| | | CTTGC | CUUGC |
| | | TTCTC | UUCUC |
| | Exon 15 SA 1 | AAGAC | AAGAC |
| | | CTAGG | CUAGG |
| | | ATTCG | AUUCG |
| | | GGGAA | GGGAA |
| | Exon 15 SA 2 | AAAGA | AAAGA |
| | | CCTAG | CCUAG |
| | | GATTC | GAUUC |
| | | GGGGA | GGGGA |
| | Exon 15 SD 1 | ACCCA | ACCCA |
| | | CCTGT | CCUGU |
| | | AGCAG | AGCAG |
| | | TGGCC | UGGCC |
| | Exon 15 SD 2 | GACCC | GACCC |
| | | ACCTG | ACCUG |
| | | TAGCA | UAGCA |
| | | GTGGC | GUGGC |
| | Exon 16 SA 1 | AGCCT | AGCCU |
| | | GGATG | GGAUG |
| | | GAGAG | GAGAG |
| | | AAACC | AAACC |
| | Exon 16 SA 2 | CAGCC | CAGCC |
| | | TGGAT | UGGAU |
| | | GGAGA | GGAGA |
| | | GAAAC | GAAAC |
| | Exon 17 SD 1 | CCTTA | CCUUA |
| | | CCTTT | CCUUU |
| | | CTGCT | CUGCU |
| | | TTCTG | UUCUG |
| | Exon 17 SD 2 | GCCTT | GCCUU |
| | | ACCTT | ACCUU |
| | | TCTGC | UCUGC |
| | | TTTCT | UUUCU |
| | Exon 17 SD 3 | GGCCT | GGCCU |
| | | TACCT | UACCU |
| | | TTCTG | UUCUG |
| | | CTTTC | CUUUC |
| | Exon 18 SA 1 | CACCC | CACCC |
| | | TGGAG | UGGAG |
| | | ATGGA | AUGGA |
| | | GGTGG | GGUGG |
| | Exon 18 SA 2 | CTTCA | CUUCA |
| | | CCCTG | CCCUG |
| | | GAGAT | GAGAU |
| | | GGAGG | GGAGG |
| | Exon 19 SD | ACAAA | ACAAA |
| | | CCCAG | CCCAG |
| | | CAGCA | CAGCA |
| | | CCATG | CCAUG |
| | Exon 20 SA 1 | GCGCC | GCGCC |
| | | TGGGG | UGGGG |
| | | AACGA | AACGA |
| | | AGAGG | AGAGG |
| | Exon 20 SA 2 | GGCGC | GGCGC |
| | | CTGGG | CUGGG |
| | | GAACG | GAACG |
| | | AAGAG | AAGAG |
| | Exon 20 SA 3 | CGGCG | CGGCG |
| | | CCTGG | CCUGG |
| | | GGAAC | GGAAC |
| | | GAAGA | GAAGA |
| | Exon 20 SA 4 | ACGGC | ACGGC |
| | | GCCTG | GCCUG |
| | | GGGAA | GGGAA |
| | | CGAAG | CGAAG |
| | Exon 20 SD | CCGTA | CCGUA |
| | | CCTGC | CCUGC |
| | | GGTGG | GGUGG |
| | | TCAGC | UCAGC |
| | Exon 23 SA | AGACG | AGACG |
| | | CCTGG | CCUGG |
| | | GGGCC | GGGCC |
| | | GGGGG | GGGGG |
| | Exon 24 SD | ACTCA | ACUCA |
| | | CAGAA | CAGAA |
| | | CACGC | CACGC |
| | | TGGCC | UGGCC |
| | Exon 25 SD | CACCT | CACCU |
| | | ACCTG | ACCUG |
| | | CCCTT | CCCUU |
| | | CCAGC | CCAGC |
| | Exon 26 SA | AAGAC | AAGAC |
| | | CTGAT | CUGAU |
| | | GAGAG | GAGAG |
| | | ACCAC | ACCAC |
| | Exon 28 SA | GCAGG | GCAGG |
| | | TCTGC | UCUGC |
| | | AGGGG | AGGGG |
| | | AGGGA | AGGGA |
| ELOB | Ex2 SA1 | ACGTC | ACGUC |
| (TCEB2) | (Pos 6) | CTGGG | CUGGG |
| | | GGCGG | GGCGG |
| | | CGGGC | CGGGC |
| | Ex3 SA1 | GTCAT | GUCAU |
| | (Pos 7) | CCTGA | CCUGA |
| | | GGAGA | GGAGA |
| | | GAAGC | GAAGC |
| | Ex4 SD1 | TCACT | UCACU |
| | (Pos 4) | GCACG | GCACG |
| | | GCTTG | GCUUG |
| | | TTCAT | UUCAU |
| | Ex5 SA1 | ATGCA | AUGCA |
| | (Pos 8) | GGCTA | GGCUA |
| | | TGGGG | UGGGG |
| | | GTGGG | GUGGG |
| | Ex5 SA2 | CATGC | CAUGC |
| | (Pos 9) | AGGCT | AGGCU |
| | | ATGGG | AUGGG |
| | | GGTGG | GGUGG |
| | Isoform ATG | GTCTT | GUCUU |
| | | TTTCA | UUUCA |
| | | TTGAC | UUGAC |
| | | TAGAA | UAGAA |
| | Exon 2 SD | TGACT | UGACU |
| | | TACCT | UACCU |
| | | TAACG | UAACG |
| | | TTTTC | UUUUC |
| | Exon 3 STOP | TGCAT | UGCAU |
| | | CAAGT | CAAGU |
| | | AGAAG | AGAAG |
| | | AATGC | AAUGC |
| | Isoform 2 ATG | CTCTT | CUCUU |
| | | TCCAT | UCCAU |
| | | GCAAT | GCAAU |
| | | CAGTC | CAGUC |
| | Exon 4 STOP | GTTCA | GUUCA |
| | | GAAAG | GAAAG |
| | | TAAAT | UAAAU |
| | | GAAAT | GAAAU |
| ENTPD1 | Isoform 3 ATG | TCACT | UCACU |
| (CD39) | | TTCCA | UUCCA |
| | | TCCTG | UCCUG |
| | | TACAA | UACAA |
| | Exon 5 STOP | GGCCA | GGCCA |
| | | AGAGG | AGAGG |
| | | AAGGT | AAGGU |
| | | GCCTA | GCCUA |
| | Exon 6 STOP 1 | GCTCT | GCUCU |
| | | GCAAT | GCAAU |
| | | TTCGC | UUCGC |
| | | CTCTA | CUCUA |
| | Exon 6 STOP 2 | ACTCT | ACUCU |
| | | GGCAG | GGCAG |
| | | AAACT | AAACU |
| | | GGCCA | GGCCA |
| | Exon 6 SD | TATAC | UAUAC |
| | | TTGCC | UUGCC |
| | | TGAAT | UGAAU |
| | | GTCCT | GUCCU |
| | Exon 7 SD | AACTT | AACUU |
| | | ACCCC | ACCCC |
| | | AAAAT | AAAAU |
| | | CCCCC | CCCCC |
| | Exon 8 STOP | CTGTG | CUGUG |
| | | CTCAG | CUCAG |
| | | CCTTG | CCUUG |
| | | GGAGG | GGAGG |
| | Exon 9 SA 4 | TTTTA | UUUUA |
| | | TCTAG | UCUAG |
| | | AAGTG | AAGUG |
| | | AAGTG | AAGUG |
| | Exon 9 SA 3 | TTTAT | UUUAU |
| | | CTAGA | CUAGA |
| | | AGTGA | AGUGA |
| | | AGTGA | AGUGA |
| | Exon 9 SA 2 | TTATC | UUAUC |
| | | TAGAA | UAGAA |
| | | GTGAA | GUGAA |
| | | GTGAG | GUGAG |
| | Exon 9 SA 1 | TATCT | UAUCU |
| | | AGAAG | AGAAG |
| | | TGAAG | UGAAG |
| | | TGAGG | UGAGG |
| | Exon 9 SD | AAATT | AAAUU |
| | | ACCTT | ACCUU |
| | | GCCAA | GCCAA |
| | | TGAAA | UGAAA |
| FADD | Exon 1 SD | CCCAC | CCCAC |
| | | CTTCT | CUUCU |
| | | TCCCC | UCCCC |
| | | AGGCG | AGGCG |
| | Exon 1 STOP | GAGCA | GAGCA |
| | | GAACG | GAACG |
| | | ACCTG | ACCUG |
| | | GAGCC | GAGCC |
| | Exon 2 STOP 1 | GTCCT | GUCCU |
| | | GCCAG | GCCAG |
| | | ATGAA | AUGAA |
| | | CCTGG | CCUGG |
| | Exon 2 STOP 2 | CCTGG | CCUGG |
| | | TACAA | UACAA |
| | | GAGGT | GAGGU |
| | | TCAGC | UCAGC |
| | Exon 2 STOP 3 | GTGAC | GUGAC |
| | | CTCCA | CUCCA |
| | | GAACA | GAACA |
| | | GGAGT | GGAGU |
| | Exon 2 STOP 4 | TGACC | UGACC |
| | | TCCAG | UCCAG |
| | | AACAG | AACAG |
| | | GAGTG | GAGUG |
| | Exon 2 STOP 5 | GTTCC | GUUCC |
| | | ATGAC | AUGAC |
| | | ATCGG | AUCGG |
| | | GGACA | GGACA |
| IL6 | Exon 1 ATG | GGAGT | GGAGU |
| | | TCATA | UCAUA |
| | | GCTGG | GCUGG |
| | | GCTCC | GCUCC |
| | Exon 2 SD | CCTAC | CCUAC |
| | | CCACC | CCACC |
| | | TCCTT | UCCUU |
| | | TCTCA | UCUCA |
| | Exon 3 SD | GTACC | GUACC |
| | | TCATT | UCAUU |
| | | GAATC | GAAUC |
| | | CAGAT | CAGAU |
| | Exon 3 STOP | CTTCC | CUUCC |
| | | AATCT | AAUCU |
| | | GGATT | GGAUU |
| | | CAATG | CAAUG |
| | Exon 4 SA 1 | CTCCT | CUCCU |
| | | AAGAG | AAGAG |
| | | GAAAG | GAAAG |
| | | ATGGT | AUGGU |
| | Exon 4 SA 2 | TCTCC | UCUCC |
| | | TAAGA | UAAGA |
| | | GGAAA | GGAAA |
| | | GATGG | GAUGG |
| | Exon 4 SA 3 | AAGTC | AAGUC |
| | | TCCTA | UCCUA |
| | | AGAGG | AGAGG |
| | | AAAGA | AAAGA |
| | Exon 4 SD | CCACC | CCACC |
| | | TTTTT | UUUUU |
| | | CTGCA | CUGCA |
| | | GGAAC | GGAAC |
| | Exon 5 STOP 1 | AGCTG | AGCUG |
| | | CAGGC | CAGGC |
| | | ACAGA | ACAGA |
| | | ACCAG | ACCAG |
| | Exon 5 STOP 2 | GGCAC | GGCAC |
| | | AGAAC | AGAAC |
| | | CAGTG | CAGUG |
| | | GCTGC | GCUGC |
| | Exon 5 STOP 3 | GTTCC | GUUCC |
| | | TGCAG | UGCAG |
| | | TCCAG | UCCAG |
| | | CCTGA | CCUGA |
| | Iso 1 Exon 2 SD | TGGGG | UGGGG |
| | | GTACT | GUACU |
| | | GGGGC | GGGGC |
| | | AGGGA | AGGGA |
| | Iso 1 Exon 4 | ATTCC | AUUCC |
| | STOP | CTCAA | CUCAA |
| | | CTTGG | CUUGG |
| | | TGTGG | UGUGG |
| IL6R | Exon 1 ATG | CGGCC | CGGCC |
| | | AGCAT | AGCAU |
| | | GCTTC | GCUUC |
| | | CTCCT | CUCCU |
| | Exon 4 SA | TGACT | UGACU |
| | | GTTAG | GUUAG |
| | | ACACA | ACACA |
| | | AAACA | AAACA |
| | Exon 4 STOP 1 | CTCCT | CUCCU |
| | | GCCAG | GCCAG |
| | | TTAGC | UUAGC |
| | | AGTCC | AGUCC |
| | Exon 4 STOP 2 | ACTCA | ACUCA |
| | | AACCT | AACCU |
| | | TTCAG | UUCAG |
| | | GGTTG | GGUUG |
| | Exon 5 STOP | CCTGG | CCUGG |
| | | CAAGA | CAAGA |
| | | CCCCC | CCCCC |
| | | ACTCC | ACUCC |
| | Exon 6 SA | TTGAC | UUGAC |
| | | CTGAG | CUGAG |
| | | GGCGG | GGCGG |
| | | GGGCA | GGGCA |
| | Exon 6 STOP 1 | CGTGG | CGUGG |
| | | TGCAG | UGCAG |
| | | CTTCG | CUUCG |
| | | TGCCC | UGCCC |
| | Exon 6 STOP 2 | ACCTG | ACCUG |
| | | TCCAA | UCCAA |
| | | GGCGT | GGCGU |
| | | GCCCA | GCCCA |
| | Exon 7 STOP | CTGGG | CUGGG |
| | | TCCCA | UCCCA |
| | | AATGC | AAUGC |
| | | CACCC | CACCC |
| | Exon 8 SA | GGATT | GGAUU |
| | | CTGCG | CUGCG |
| | | GACAG | GACAG |
| | | AAGAA | AAGAA |
| | Exon 8 SD | GGAGC | GGAGC |
| | | TCACC | UCACC |
| | | TGCAT | UGCAU |
| | | GGGGG | GGGGG |
| IL10 | Exon 2 SA | TTTGC | UUUGC |
| | | TGCAG | UGCAG |
| | | GAAGA | GAAGA |
| | | ACAAA | ACAAA |
| | Exon2 STOP | GCAGC | GCAGC |
| | | AAATG | AAAUG |
| | | AAGGA | AAGGA |
| | | TCAGC | UCAGC |
| | Exon 3 SA | ACCCT | ACCCU |
| | | AAGGG | AAGGG |
| | | CAGGA | CAGGA |
| | | GCCAA | GCCAA |
| | Exon 3 STOP 1 | GATGA | GAUGA |
| | | TCCAG | UCCAG |
| | | TTTTA | UUUUA |
| | | CCTGG | CCUGG |
| | Exon 3 STOP 2 | GAACC | GAACC |
| | | AAGAC | AAGAC |
| | | CCAGA | CCAGA |
| | | CATCA | CAUCA |
| IL10RA | Ex 5 STOP | CCAGG | CCAGG |
| | (pos 6) | CAGTG | CAGUG |
| | | TGAGT | UGAGU |
| | | CAGCT | CAGCU |
| | Ex 6 STOP | TGGCC | UGGCC |
| | (pos 9) | CTCCA | CUCCA |
| | | GCTGT | GCUGU |
| | | ATGTG | AUGUG |
| | Ex2 STOP | CTTCA | CUUCA |
| | (pos 8) | AACCA | AACCA |
| | | CACAG | CACAG |
| | | ACGGA | ACGGA |
| | Ex2 STOP | TGGGT | UGGGU |
| | (pos 8) | GTCCA | GUCCA |
| | | GTGGA | GUGGA |
| | | GGATG | GGAUG |
| | Ex2 STOP | GCTTC | GCUUC |
| | (pos 9) | AAACC | AAACC |
| | | ACACA | ACACA |
| | | GACGG | GACGG |
| | Ex3 SA | TCCAT | UCCAU |
| | (pos 8) | ACCTG | ACCUG |
| | | AGGAG | AGGAG |
| | | ATACC | AUACC |
| | Ex3 STOP | TGACG | UGACG |
| | (pos 8) | GTCCA | GUCCA |
| | | GTTGG | GUUGG |
| | | AGTGC | AGUGC |
| | Ex4 SD | CCCCA | CCCCA |
| | (pos 8) | TACCG | UACCG |
| | | TGAAG | UGAAG |
| | | TTTCC | UUUCC |
| | Ex5 SD | TGACT | UGACU |
| | (pos 8) | CACAC | CACAC |
| | | TGCCT | UGCCU |
| | | GGTGA | GGUGA |
| | Ex5 SD | CTGAC | CUGAC |
| | (pos 9) | TCACA | UCACA |
| | | CTGCC | CUGCC |
| | | TGGTG | UGGUG |
| | Ex5 STOP | ACCAG | ACCAG |
| | (pos 7) | GCAGT | GCAGU |
| | | GTGAG | GUGAG |
| | | TCAGC | UCAGC |
| | Ex7 SA | TTGAA | UUGAA |
| | (pos 9) | GAGCT | GAGCU |
| | | GGGGA | GGGGA |
| | | AGAGA | AGAGA |
| | Ex7 STOP | AGCTC | AGCUC |
| | (pos 5) | CAGTC | CAGUC |
| | | AGATA | AGAUA |
| | | TTCCC | UUCCC |
| | Ex7 STOP | AGTTC | AGUUC |
| | (pos 5) | AAAAC | AAAAC |
| | | TCTGA | UCUGA |
| | | GGGCC | GGGCC |
| | Ex7 STOP | TAGTT | UAGUU |
| | (pos 6) | CAAAA | CAAAA |
| | | CTCTG | CUCUG |
| | | AGGGC | AGGGC |
| | Ex7 STOP | CTGGT | CUGGU |
| | (pos 7) | TCCAC | UCCAC |
| | | TGTCC | UGUCC |
| | | CGTTG | CGUUG |
| | Ex7 STOP | GCTGG | GCUGG |
| | (pos 8) | TTCCA | UUCCA |
| | | CTGTC | CUGUC |
| | | CCGTT | CCGUU |
| | Ex7 STOP | TGGCA | UGGCA |
| | (pos 9) | TTCCA | UUCCA |
| | | GGGTT | GGGUU |
| | | ACCTG | ACCUG |
| | Ex7 STOP | GGCTG | GGCUG |
| | (pos 9) | GTTCC | GUUCC |
| | | ACTGT | ACUGU |
| | | CCCGT | CCCGU |
| IRF4 | Exon 1 SD | GCCGG | GCCGG |
| | (pos 9) | AGACC | AGACC |
| | | TTGAA | UUGAA |
| | | GAGCG | GAGCG |
| | Exon 1 STOP 1 | GGCGG | GGCGG |
| | (pos 7) | CCGAG | CCGAG |
| | | GCGGA | GCGGA |
| | | GAGTT | GAGUU |
| | Exon 1 STOP 2 | CGTTC | CGUUC |
| | (pos 8/9) | TCCCA | UCCCA |
| | | CACCA | CACCA |
| | | GCCCG | GCCCG |
| | Exon 1 STOP 3 | CGCGG | CGCGG |
| | (pos 5) | TTGTA | UUGUA |
| | | GTCCT | GUCCU |
| | | GCTTG | GCUUG |
| | Exon 2 SD 1 | CTACC | CUACC |
| | | TTTTT | UUUUU |
| | | TGGCT | UGGCU |
| | | CCCTC | CCCUC |
| | Exon 2 STOP 1 | GTCTT | GUCUU |
| | | CCAGG | CCAGG |
| | | TGGGA | UGGGA |
| | | GGGTC | GGGUC |
| | Exon 3 SA 1 | CTCCT | CUCCU |
| | | ACATG | ACAUG |
| | | TTTGG | UUUGG |
| | | GGAAA | GGAAA |
| | Exon 3 SD 1 | ATACC | AUACC |
| | | TGGGC | UGGGC |
| | | TGGGA | UGGGA |
| | | GCGAA | GCGAA |
| | Exon 3 SD 2 | CATAC | CAUAC |
| | | CTGGG | CUGGG |
| | | CTGGG | CUGGG |
| | | AGCGA | AGCGA |
| | Exon 3 STOP 1 | AGCCA | AGCCA |
| | | AGCAG | AGCAG |
| | | CTCAC | CUCAC |
| | | CCTGG | CCUGG |
| | Exon 3 STOP 2 | CCCAG | CCCAG |
| | | CCCAG | CCCAG |
| | | GTATG | GUAUG |
| | | GTGGA | GUGGA |
| | Exon 4 SA 1 | CTGCT | CUGCU |
| | | AAAGG | AAAGG |
| | | AGTGC | AGUGC |
| | | AGGAG | AGGAG |
| | Exon 4 SD 1 | TCCTT | UCCUU |
| | | ACCAT | ACCAU |
| | | TTTCA | UUUCA |
| | | CAAGC | CAAGC |
| | Exon 4 SD 2 | CCTTA | CCUUA |
| | | CCATT | CCAUU |
| | | TTCAC | UUCAC |
| | | AAGCT | AAGCU |
| | Exon 4 STOP 1 | AGTCC | AGUCC |
| | | CTCCA | CUCCA |
| | | GCTTC | GCUUC |
| | | GGTCG | GGUCG |
| | Exon 4 STOP 2 | GTCCC | GUCCC |
| | | TCCAG | UCCAG |
| | | CTTCG | CUUCG |
| | | GTCGA | GUCGA |
| | Exon 4 STOP 3 | TCCCT | UCCCU |
| | | CCAGC | CCAGC |
| | | TTCGG | UUCGG |
| | | TCGAG | UCGAG |
| | Exon 4 STOP 4 | TACCA | UACCA |
| | | ATGTC | AUGUC |
| | | CCATG | CCAUG |
| | | ACGTT | ACGUU |
| | Exon 4 STOP 5 | GCCTT | GCCUU |
| | | GCCAG | GCCAG |
| | | TGGTG | UGGUG |
| | | GCCGC | GCCGC |
| | Exon 4 STOP 6 | CCTTG | CCUUG |
| | | CCAGT | CCAGU |
| | | GGTGG | GGUGG |
| | | CCGCG | CCGCG |
| | Exon 5 SD 1 | GCACT | GCACU |
| | | CACCT | CACCU |
| | | GAGAA | GAGAA |
| | | CGCCA | CGCCA |
| | Exon 6 SA 1 | AGTCT | AGUCU |
| | | GCAAA | GCAAA |
| | | CACAG | CACAG |
| | | AGCTC | AGCUC |
| | Exon 6 STOP 1 | TGTGC | UGUGC |
| | | CAGAG | CAGAG |
| | | CAGGA | CAGGA |
| | | TCTAC | UCUAC |
| | Exon 6 STOP 2 | GTGCC | GUGCC |
| | | AGAGC | AGAGC |
| | | AGGAT | AGGAU |
| | | CTACT | CUACU |
| | Exon 6 STOP 3 | GACAC | GACAC |
| | | ACAGC | ACAGC |
| | | AGTTC | AGUUC |
| | | TTGTC | UUGUC |
| | Exon 7 STOP 1 | CTGCA | CUGCA |
| | | AGCGT | AGCGU |
| | | TTGCT | UUGCU |
| | | CACCA | CACCA |
| | Exon 7 STOP 2 | TTCCA | UUCCA |
| | | GGTGA | GGUGA |
| | | CTCTA | CUCUA |
| | | TGCTT | UGCUU |
| IRF8 | Ex1 SA | CCTTC | CCUUC |
| | (Pos 7) | TCATG | UCAUG |
| | | GCAGG | GCAGG |
| | | TGTCC | UGUCC |
| | Ex1 SD | CCCAC | CCCAC |
| | (Pos 5) | AGATT | AGAUU |
| | | CAGGG | CAGGG |
| | | ACTCC | ACUCC |
| | Ex1 SD | ACCCA | ACCCA |
| | (Pos 6) | CAGAT | CAGAU |
| | | TCAGG | UCAGG |
| | | GACTC | GACUC |
| | Ex2 SD | GCTCT | GCUCU |
| | (Pos 9) | TTACC | UUACC |
| | | TTAAA | UUAAA |
| | | AATGG | AAUGG |
| | Ex3 SD | TTACA | UUACA |
| | (Pos 4) | TTTTT | UUUUU |
| | | GCTCT | GCUCU |
| | | TCCTC | UCCUC |
| | Ex4 SA | GAAGG | GAAGG |
| | (Pos 6) | CTGCA | CUGCA |
| | | CAGTC | CAGUC |
| | | AGGGG | AGGGG |
| | Ex4 SA | AAGGC | AAGGC |
| | (Pos 5) | TGCAC | UGCAC |
| | | AGTCA | AGUCA |
| | | GGGGA | GGGGA |
| | Ex4 SA | ACAGA | ACAGA |
| | (Pos 9) | AGGCT | AGGCU |
| | | GCACA | GCACA |
| | | GTCAG | GUCAG |
| | Ex5 SA1 | CACCA | CACCA |
| | (Pos 7) | TCTGG | UCUGG |
| | | GAGAA | GAGAA |
| | | TGCTG | UGCUG |
| | Ex5 SA2 | GGAGA | GGAGA |
| | (Pos 9) | ATGCT | AUGCU |
| | | GTGGA | GUGGA |
| | | CAAGA | CAAGA |
| | Ex5 SD1 | GGTAC | GGUAC |
| | (Pos 9) | AGACC | AGACC |
| | | TCGGA | UCGGA |
| | | AGAAC | AGAAC |
| | Ex5 SD2 | CAGAC | CAGAC |
| | (Pos 5) | CTCGG | CUCGG |
| | | AAGAA | AAGAA |
| | | CTGGC | CUGGC |
| | Ex6 SA1 | CTGCA | CUGCA |
| | (Pos 9) | GCTCT | GCUCU |
| | | GGAAT | GGAAU |
| | | GACAC | GACAC |
| JUNB | Ex1 SA1 | GCACA | GCACA |
| | (Pos 7) | TCCGG | UCCGG |
| | | GCGGC | GCGGC |
| | | CCAGG | CCAGG |
| | Ex1 STOP1 | GGATA | GGAUA |
| | (Pos 9) | CGGCC | CGGCC |
| | | GGGCC | GGGCC |
| | | CCTGG | CCUGG |
| | Ex1 STOP2 | TCTGG | UCUGG |
| | (Pos 7) | TCAGG | UCAGG |
| | | GCTCG | GCUCG |
| | | GACAC | GACAC |
| | Ex1 STOP2 | GGACA | GGACA |
| | (Pos 4) | GTACT | GUACU |
| | | TTTAC | UUUAC |
| | | CCCCG | CCCCG |
| | Ex1 STOP3 | GAGCA | GAGCA |
| | (Pos 4) | GGAGG | GGAGG |
| | | GCTTC | GCUUC |
| | | GCCGA | GCCGA |
| | Ex1 STOP4 | GCGCA | GCGCA |
| | (Pos 4) | GCTGG | GCUGG |
| | | GCTTG | GCUUG |
| | | GGCCG | GGCCG |
| | Ex1 STOP6 | GGAAC | GGAAC |
| | (Pos 8) | CGCAG | CGCAG |
| | | ACCGT | ACCGU |
| | | GCCGG | GCCGG |
| | EX1 STOP7 | CAGCC | CAGCC |
| | (Pos 5) | GGGAC | GGGAC |
| | | GCCAC | GCCAC |
| | | GCCGC | GCCGC |
| | Ex1 STOP8 | AGACC | AGACC |
| | (Pos 5) | AAGAG | AAGAG |
| | | CGCAT | CGCAU |
| | | CAAAG | CAAAG |
| | Ex1 STOP9 | CAAGC | CAAGC |
| | (Pos 5) | GGCTG | GGCUG |
| | | CGGAA | CGGAA |
| | | CCGGC | CCGGC |
| | Ex1 STOP10 | GCGGC | GCGGC |
| | (Pos 8) | TGCGG | UGCGG |
| | | AACCG | AACCG |
| | | GCTGG | GCUGG |
| LAIR-1 | Exon 1 ATG | TCTCT | UCUCU |
| (CD305 | | TTCCA | UUCCA |
| | | TCTTC | UCUUC |
| | | TGTCG | UGUCG |
| | Iso 1 Ex 2 | ATGAC | AUGAC |
| | SD 2 | TTACC | UUACC |
| | | CTCCT | CUCCU |
| | | GCGTG | GCGUG |
| | Iso 1 Ex 2 | CTTAC | CUUAC |
| | SD 1 | CCTCC | CCUCC |
| | | TGCGT | UGCGU |
| | | GTGGA | GUGGA |
| | Exon 2 STOP | TGGGG | UGGGG |
| | | TTCAA | UUCAA |
| | | ACATT | ACAUU |
| | | CCGCC | CCGCC |
| | Exon 3 SA | AGCTT | AGCUU |
| | | TCTGT | UCUGU |
| | | AAACA | AAACA |
| | | GGGGC | GGGGC |
| | Exon 3 SD 1 | TACTG | UACUG |
| | | ACCAG | ACCAG |
| | | CTGAG | CUGAG |
| | | GAGCC | GAGCC |
| | Exon 3 SD 2 | CTACT | CUACU |
| | | GACCA | GACCA |
| | | GCTGA | GCUGA |
| | | GGAGC | GGAGC |
| | Exon 5 SA | CATCT | CAUCU |
| | | AAGAA | AAGAA |
| | | AGACA | AGACA |
| | | GAAAC | GAAAC |
| | Exon 6 SA 1 | GGGGG | GGGGG |
| | | CCCTA | CCCUA |
| | | AGGAC | AGGAC |
| | | AGTCG | AGUCG |
| | Exon 6 SA 2 | GGGGG | GGGGG |
| | | GCCCT | GCCCU |
| | | AAGGA | AAGGA |
| | | CAGTC | CAGUC |
| | Exon 8 SA | TGTCT | UGUCU |
| | | TGGGG | UGGGG |
| | | AGAAA | AGAAA |
| | | ATACA | AUACA |
| | Exon 9 SA | GGCCT | GGCCU |
| | | AAGAG | AAGAG |
| | | GGAGA | GGAGA |
| | | GACCC | GACCC |
| LDHA | Ex0 SD1 | CCCAT | CCCAU |
| | (Pos 7) | ACCTT | ACCUU |
| | | AGCGT | AGCGU |
| | | GGAAA | GGAAA |
| | Ex4 STOP | ACCCA | ACCCA |
| | (Pos 4) | CCCAT | CCCAU |
| | | GACAG | GACAG |
| | | CTTAA | CUUAA |
| | Ex6 SD1 | TAGAC | UAGAC |
| | (Pos 9) | CTACC | CUACC |
| | | TTAAT | UUAAU |
| | | CATGG | CAUGG |
| LIF | Ex2 SA1 | CACAA | CACAA |
| | (Pos 9) | CTCCT | CUCCU |
| | | GGGGA | GGGGA |
| | | CAGTC | CAGUC |
| | Ex2 SD1 | AACTT | AACUU |
| | (Pos 7) | ACATA | ACAUA |
| | | GAGAA | GAGAA |
| | | TAAAG | UAAAG |
| | Ex2 SD2 | ACTTA | ACUUA |
| | (Pos 6) | CATAG | CAUAG |
| | | AGAAT | AGAAU |
| | | AAAGA | AAAGA |
| | Ex2 STOP1 | GAACC | GAACC |
| | (Pos 5) | AGATC | AGAUC |
| | | AGGAG | AGGAG |
| | | CCAAC | CCAAC |
| | Ex4 SA1 | CTGTG | CUGUG |
| | (Pos 8) | TACTG | UACUG |
| | | AGGGG | AGGGG |
| | | CAGAA | CAGAA |
| | Ex4 SA2 | GCTGT | GCUGU |
| | (Pos 9) | GTACT | GUACU |
| | | GAGGG | GAGGG |
| | | GCAGA | GCAGA |
| | Ex4 SA3 | TGTAC | UGUAC |
| | (Pos 5) | TGAGG | UGAGG |
| | | GGCAG | GGCAG |
| | | AAGGG | AAGGG |
| LYN | Ex1 STOP1 | GGTGC | GGUGC |
| | (Pos 8) | TCCCA | UCCCA |
| | | GGAGC | GGAGC |
| | | GGAGG | GGAGG |
| | Ex4 STOP 1 | AGAGG | AGAGG |
| | (Pos 8) | AACAA | AACAA |
| | | GGAGA | GGAGA |
| | | CATTG | CAUUG |
| | Ex5 SD1 | GACTC | GACUC |
| | (Pos 5) | ACTCT | ACUCU |
| | | TCTGT | UCUGU |
| | | TTCTA | UUCUA |
| | Ex6 STOP1 | GAAAG | GAAAG |
| | (Pos 7) | GCAGC | GCAGC |
| | | TTTTG | UUUUG |
| | | GCACC | GCACC |
| | Ex8 STOP1 | AGCCA | AGCCA |
| | (Pos 4) | CAGAA | CAGAA |
| | | GCCAT | GCCAU |
| | | GGGAT | GGGAU |
| | Ex8 STOP2 | CCCCC | CCCCC |
| | (Pos 5) | GGGAG | GGGAG |
| | | TCCAT | UCCAU |
| | | CAAGT | CAAGU |
| | Ex9 SA1 | TAACC | UAACC |
| | (Pos 5) | TAGGA | UAGGA |
| | | AGAAA | AGAAA |
| | | AAAGA | AAAGA |
| | Ex9 SD1 | CTCAC | CUCAC |
| | (Pos 5) | CCTTG | CCUUG |
| | | GCCAT | GCCAU |
| | | GTACT | GUACU |
| | Ex10 SA1 | CCTGA | CCUGA |
| | (Pos 7) | ACTGG | ACUGG |
| | | AGGTG | AGGUG |
| | | AACAA | AACAA |
| | Ex11 SA1 | TGCAA | UGCAA |
| | (Pos 7) | TCTGA | UCUGA |
| | | AAACA | AAACA |
| | | GAAAT | GAAAU |
| | Ex12 SA1 | CACCT | CACCU |
| | (Pos 4) | AAGGA | AAGGA |
| | | AGAAG | AGAAG |
| | | ATATG | AUAUG |
| | Ex13 STOP1 | AGCAG | AGCAG |
| | (Pos 6) | CAGCC | CAGCC |
| | | TTAGA | UUAGA |
| | | GCACA | GCACA |
| MAP4K4 | Ex1 SD1 | CACTC | CACUC |
| | (Pos 7) | ACCCG | ACCCG |
| | | CAGGG | CAGGG |
| | | AGGAG | AGGAG |
| | Ex2 SA1 | AGGAT | AGGAU |
| | (Pos 7) | CCTGG | CCUGG |
| | | AGAGG | AGAGG |
| | | AAGGA | AAGGA |
| | Ex2 SA2 | GGATC | GGAUC |
| | (Pos 6) | CTGGA | CUGGA |
| | | GAGGA | GAGGA |
| | | AGGAG | AGGAG |
| | Ex4 STOP1 | GATGA | GAUGA |
| | (Pos 7) | CCAAC | CCAAC |
| | | TCTGG | UCUGG |
| | | GTAGG | GUAGG |
| | Ex5 SD1 | CCTTA | CCUUA |
| | (Pos 6) | CCCTC | CCCUC |
| | | AGGAT | AGGAU |
| | | TTCTC | UUCUC |
| | Ex7 STOP1 | GTGAT | GUGAU |
| | (Pos 9) | TCACC | UCACC |
| | | GGGAT | GGGAU |
| | | ATCAA | AUCAA |
| | Ex8 SA1 | CAACT | CAACU |
| | (Pos 4) | GTGGG | GUGGG |
| | | AGGAA | AGGAA |
| | | GAAAA | GAAAA |
| | Ex8 SD1 | GCCTC | GCCUC |
| | (Pos 9) | TTACT | UUACU |
| | | CTGTA | CUGUA |
| | | ATCAT | AUCAU |
| | Ex8 SD2 | TCTTA | UCUUA |
| | (Pos 6) | CTCTG | CUCUG |
| | | TAATC | UAAUC |
| | | ATAGG | AUAGG |
| | Ex10 SA1 | AGAGA | AGAGA |
| | (Pos 7) | GCTGG | GCUGG |
| | | GGAGA | GGAGA |
| | | GGAGA | GGAGA |
| | Ex12 STOP1 | CTGAA | CUGAA |
| | (Pos 6) | CAGGA | CAGGA |
| | | AGGAG | AGGAG |
| | | AGCCA | AGCCA |
| | Ex13 SA1 | ATGGA | AUGGA |
| | (Pos 7) | ACTGT | ACUGU |
| | | TGGAA | UGGAA |
| | | AAAGC | AAAGC |
| | Ex13 STOP2 | TTGAG | UUGAG |
| | (Pos 6) | CAGCA | CAGCA |
| | | GAAAG | GAAAG |
| | | AACAG | AACAG |
| | Ex14 STOP1 | GGAGA | GGAGA |
| | (Pos 9) | GAGCG | GAGCG |
| | | GGAAG | GGAAG |
| | | CTAGA | CUAGA |
| | Ex15 STOP1 | CCTTC | CCUUC |
| | (Pos 5) | AGCAG | AGCAG |
| | | CAGCT | CAGCU |
| | | GCTCC | GCUCC |
| | Ex16 SA1 | GGCAC | GGCAC |
| | (Pos 8) | TCCTT | UCCUU |
| | | GGAGA | GGAGA |
| | | GGGAG | GGGAG |
| | Ex16 STOP1 | CTCCC | CUCCC |
| | (Pos 7) | GCCAT | GCCAU |
| | | CGGCA | CGGCA |
| | | CTCCT | CUCCU |
| | Ex17 STOP1 | GAAGC | GAAGC |
| | (Pos 7) | CCAGT | CCAGU |
| | | CTAAG | CUAAG |
| | | CAGAC | CAGAC |
| | Ex17 SD1 | GTCGC | GUCGC |
| | (Pos 8) | TACCT | UACCU |
| | | GTGGC | GUGGC |
| | | TCCGC | UCCGC |
| | Ex18 STOP1 | TAGAC | UAGAC |
| | (Pos 5) | CAAGC | CAAGC |
| | | CTTTT | CUUUU |
| | | GGGT | GGGUA |
| | | A | |
| | Ex18 STOP1 | GGGCA | GGGCA |
| | (Pos 4) | GCAGA | GCAGA |
| | | ATAGC | AUAGC |
| | | CAGGC | CAGGC |
| | Ex19 STOP1 | AGGCT | AGGCU |
| | (Pos 9) | TCTGT | UCUGU |
| | | GGGAG | GGGAG |
| | | AGAGT | AGAGU |
| | Ex19 STOP2 | TGGGT | UGGGU |
| | (Pos 8) | CTCAG | CUCAG |
| | | AGTGG | AGUGG |
| | | CTCCG | CUCCG |
| | Ex20 SA1 | ATGAT | AUGAU |
| | (Pos 7) | GCTGT | GCUGU |
| | | TGGGT | UGGGU |
| | | TCAAA | UCAAA |
| | Ex20 SA2 | TGATG | UGAUG |
| | (Pos 6) | CTGTT | CUGUU |
| | | GGGTT | GGGUU |
| | | CAAAA | CAAAA |
| | Ex20 SD1 | ATCCT | AUCCU |
| | (Pos 8) | TACAG | UACAG |
| | | CAGGC | CAGGC |
| | | TTGAG | UUGAG |
| | Ex20 SD2 | AATCC | AAUCC |
| | (Pos 9) | TTACA | UUACA |
| | | GCAGG | GCAGG |
| | | CTTGA | CUUGA |
| | Ex24 STOP1 | TAGGC | UAGGC |
| | (Pos 8) | CACAG | CACAG |
| | | AGTGA | AGUGA |
| | | CACCC | CACCC |
| | Ex25 STOP | CCGAA | CCGAA |
| | (Pos 8) | GACGA | GACGA |
| | | TTTCA | UUUCA |
| | | ACAAA | ACAAA |
| | Ex26 STOP1 | AAGCA | AAGCA |
| | (Pos 4) | GGGAT | GGGAU |
| | | GGACA | GGACA |
| | | ACCGT | ACCGU |
| | Ex30 STOP1 | TCCCC | UCCCC |
| | (Pos 5) | AGCCC | AGCCC |
| | | ATTGT | AUUGU |
| | | CTGAT | CUGAU |
| | Ex30 STOP2 | GAGAT | GAGAU |
| | (Pos 7) | CCGAT | CCGAU |
| | | CTGTG | CUGUG |
| | | GAAAC | GAAAC |
| MAPK14 | Ex1 SD1 | CACTC | CACUC |
| | (Pos 7) | ACCAC | ACCAC |
| | | ACAGA | ACAGA |
| | | GCCAT | GCCAU |
| | Ex1 STOP1 | TTACC | UUACC |
| | (Pos 5) | AGAAC | AGAAC |
| | | CTGTC | CUGUC |
| | | TCCAG | UCCAG |
| | Ex2 SA1 | AGCAG | AGCAG |
| | (Pos 8) | CACTA | CACUA |
| | | AGGAG | AGGAG |
| | | AAAAA | AAAAA |
| | Ex2 SA2 | GCAGC | GCAGC |
| | (Pos 7) | ACTAA | ACUAA |
| | | GGAGA | GGAGA |
| | | AAAAA | AAAAA |
| | Ex5 SA1 | AGGTC | AGGUC |
| | (Pos 6) | CTAGG | CUAGG |
| | | AAGCA | AAGCA |
| | | AATAC | AAUAC |
| | Ex5 SA2 | GGTCC | GGUCC |
| | (Pos 5) | TAGGA | UAGGA |
| | | AGCAA | AGCAA |
| | | ATACA | AUACA |
| | Ex6 SA1 | CAGAA | CAGAA |
| | (Pos 7) | TCTAA | UCUAA |
| | | AGGGC | AGGGC |
| | | AGAAG | AGAAG |
| | Ex6 STOP1 | ATCCA | AUCCA |
| | (Pos 4) | GTTCA | GUUCA |
| | | GCATG | GCAUG |
| | | ATCTC | AUCUC |
| | Ex7 SD1 | AATAC | AAUAC |
| | (Pos 5) | CTCAG | CUCAG |
| | | TTGCC | UUGCC |
| | | GGTGC | GGUGC |
| | Ex7 STOP1 | CCCAC | CCCAC |
| | (Pos 9) | TGACC | UGACC |
| | | AAATA | AAAUA |
| | | TCAAC | UCAAC |
| | Ex8 SA1 | TATCT | UAUCU |
| | (Pos 4) | AATGG | AAUGG |
| | | TGGAC | UGGAC |
| | | CATAA | CAUAA |
| | Ex9 SA1 | ATATC | AUAUC |
| | (Pos 5) | TTAGA | UUAGA |
| | | TGCCT | UGCCU |
| | | AGTCA | AGUCA |
| | Ex9 STOP1 | CAGCA | CAGCA |
| | (Pos 4) | GATTA | GAUUA |
| | | TGCGT | UGCGU |
| | | CTGAC | CUGAC |
| | Ex10 SA1 | TTTCT | UUUCU |
| | (Pos 9) | TGCCT | UGCCU |
| | | GAAAA | GAAAA |
| | | AACAA | AACAA |
| | Ex11 SA1 | CAGCT | CAGCU |
| | (Pos 4) | ATCAG | AUCAG |
| | | TACCA | UACCA |
| | | TAGAC | UAGAC |
| | Ex11 STOP1 | ATGAT | AUGAU |
| | (Pos 6) | CAGTC | CAGUC |
| | | CTTTG | CUUUG |
| | | AAAGC | AAAGC |
| | Ex12 SA1 | GTCAG | GUCAG |
| | (Pos 8) | GCCTA | GCCUA |
| | | GAAAT | GAAAU |
| | | TGGGA | UGGGA |
| MEF2D | Ex2 SA1 | AAGTC | AAGUC |
| | (Pos 8) | ACCTG | ACCUG |
| | | CAGAG | CAGAG |
| | | AAGGA | AAGGA |
| | Ex2 SA2 | AGTCA | AGUCA |
| | (Pos 7) | CCTGC | CCUGC |
| | | AGAGA | AGAGA |
| | | AGGAT | AGGAU |
| | Ex2 SD1 | TGGGC | UGGGC |
| | (Pos 9) | CCACC | CCACC |
| | | TCGAT | UCGAU |
| | | GATGT | GAUGU |
| | Ex3 STOP1 | GGAAC | GGAAC |
| | (Pos 5) | AGAGC | AGAGC |
| | | CCCCT | CCCCU |
| | | GCTGG | GCUGG |
| | Ex3 STOP2 | CAAGT | CAAGU |
| | (Pos 8) | ACCGA | ACCGA |
| | | CGCGC | CGCGC |
| | | CAGCG | CAGCG |
| | Ex4 SA1 | AGCGC | AGCGC |
| | (Pos 6) | CTGGG | CUGGG |
| | | GGGAA | GGGAA |
| | | GGGGC | GGGGC |
| | Ex4 STOP1 | AATGA | AAUGA |
| | (Pos 8) | TGCAG | UGCAG |
| | | AGTTA | AGUUA |
| | | TAGAC | UAGAC |
| | Ex5 SA1 | CAGTT | CAGUU |
| | (Pos 8) | GACTA | GACUA |
| | | GACAG | GACAG |
| | | AAAGA | AAAGA |
| | Ex5 SA2 | TTGAC | UUGAC |
| | (Pos 5) | TAGAC | UAGAC |
| | | AGAAA | AGAAA |
| | | GATGG | GAUGG |
| | Ex5 SA3 | TGACT | UGACU |
| | (Pos 4) | AGACA | AGACA |
| | | GAAAG | GAAAG |
| | | ATGGA | AUGGA |
| | Ex5 STOP1 | CCCAG | CCCAG |
| | (Pos 6) | CAGCC | CAGCC |
| | | AGCAC | AGCAC |
| | | TACAG | UACAG |
| | Ex6 SD1 | TGCAC | UGCAC |
| | (Pos 9) | TCACC | UCACC |
| | | AACAG | AACAG |
| | | GGCTG | GGCUG |
| | Ex6 SD2 | CTCAC | CUCAC |
| | (Pos 5) | CAACA | CAACA |
| | | GGGCT | GGGCU |
| | | GGGGC | GGGGC |
| | Ex7 STOP1 | ACCTG | ACCUG |
| | (Pos 6) | CGAGT | CGAGU |
| | | CATCA | CAUCA |
| | | CTTCC | CUUCC |
| | Ex9 SD1 | CTCAC | CUCAC |
| | (Pos 5) | CTGTG | CUGUG |
| | | TTGTA | UUGUA |
| | | GGCAG | GGCAG |
| | Ex9 SD2 | TCACC | UCACC |
| | (Pos 4) | TGTGT | UGUGU |
| | | TGTAG | UGUAG |
| | | GCAGT | GCAGU |
| | Ex10 STOP1 | ACCTC | ACCUC |
| | (Pos 5) | AGCAA | AGCAA |
| | | CAGTC | CAGUC |
| | | CCACC | CCACC |
| | Ex11 SA1 | CCCGG | CCCGG |
| | (Pos 7) | GCTGG | GCUGG |
| | | AGGCA | AGGCA |
| | | GGCAA | GGCAA |
| | Ex12 SA1 | ATGTC | AUGUC |
| | (Pos 5) | TGTGA | UGUGA |
| | | AGAGA | AGAGA |
| | | GGAGA | GGAGA |
| MGAT5 | Ex2 STOP1 | TTTGC | UUUGC |
| | (Pos 5) | AGCGC | AGCGC |
| | | ATTGG | AUUGG |
| | | CAAGT | CAAGU |
| | Ex6 STOP1 | TCCGG | UCCGG |
| | (Pos 6) | CGAAT | CGAAU |
| | | GGCTG | GGCUG |
| | | ACGCA | ACGCA |
| | Ex7 SA1 | CGAGG | CGAGG |
| | (Pos 8) | ACCTG | ACCUG |
| | | GAAAA | GAAAA |
| | | CAAAG | CAAAG |
| | Ex8 SD1 | CACTT | CACUU |
| | (Pos 7) | ACTGG | ACUGG |
| | | TAATG | UAAUG |
| | | AACCC | AACCC |
| | Ex9 STOP1 | CTCCG | CUCCG |
| | (Pos 4) | AGTCC | AGUCC |
| | | TTGAT | UUGAU |
| | | TCATT | UCAUU |
| | Ex9 STOP2 | CAGAT | CAGAU |
| | (Pos 8) | TCCAT | UCCAU |
| | | TTTCC | UUUCC |
| | | CCAAG | CCAAG |
| | Ex9 STOP3 | TCAGA | UCAGA |
| | (Pos 9) | TTCCA | UUCCA |
| | | TTTTC | UUUUC |
| | | CCCAA | CCCAA |
| | Ex10 STOP1 | GAAGG | GAAGG |
| | (Pos 7) | CCATG | CCAUG |
| | | CCTGG | CCUGG |
| | | AACAC | AACAC |
| | Ex11 SD1 | TTACC | UUACC |
| | (Pos 4) | TTGGT | UUGGU |
| | | TTCTC | UUCUC |
| | | GAAGA | GAAGA |
| | Ex12 SD1 | ATGCT | AUGCU |
| | (Pos 8) | TACCT | UACCU |
| | | CTCTC | CUCUC |
| | | AGAGT | AGAGU |
| | Ex13 STOP1 | CAACA | CAACA |
| | (Pos 8) | ATCAG | AUCAG |
| | | GAGGA | GAGGA |
| | | AGTAG | AGUAG |
| | Ex15 STOP1 | GTGGC | GUGGC |
| | (Pos 5) | CACAT | CACAU |
| | | CACTT | CACUU |
| | | GCCCA | GCCCA |
| | Ex15 STOP2 | GCCCG | GCCCG |
| | (Pos 8) | GGCAG | GGCAG |
| | | TCCTG | UCCUG |
| | | CAAGC | CAAGC |
| | Ex16 SA1 | GTACC | GUACC |
| | (Pos 5) | TGAAG | UGAAG |
| | | AGGAA | AGGAA |
| | | GAGAA | GAGAA |
| | Ex16 STOP2 | CCCTG | CCCUG |
| | (Pos 6) | CCGGG | CCGGG |
| | | ACTTC | ACUUC |
| | | ATCAA | AUCAA |
| NT5E | Ex1 SD1 | TTACC | UUACC |
| (CD73) | (Pos 4) | ATGGC | AUGGC |
| | | ATCGT | AUCGU |
| | | AGCGC | AGCGC |
| | Ex1 STOP | AGGCC | AGGCC |
| | (Pos 4) | ACAGC | ACAGC |
| | | ACCGC | ACCGC |
| | | GCCCA | GCCCA |
| | Ex3 STOP1 | CGCTC | CGCUC |
| | (Pos 5) | AGAAA | AGAAA |
| | | GTGAG | GUGAG |
| | | GGGTG | GGGUG |
| | Ex4 STOP1 | GTAGT | GUAGU |
| | (Pos 7) | CCAGG | CCAGG |
| | | CCTAT | CCUAU |
| | | GCTTT | GCUUU |
| | Ex5 SA1 | GATCT | GAUCU |
| | (Pos 4) | AGAAG | AGAAG |
| | | AAAGA | AAAGA |
| | | AAAGA | AAAGA |
| | Ex5 SD1 | TTACC | UUACC |
| | (Pos 5) | ATTGC | AUUGC |
| | | ATCAC | AUCAC |
| | | AAATC | AAAUC |
| | Ex7 SD1 | GTGAC | GUGAC |
| | (Pos 9) | TTACC | UUACC |
| | | GCCCA | GCCCA |
| | | CCTGC | CCUGC |
| | Ex7 STOP1 | CTCCC | CUCCC |
| | (Pos 4) | AGGTA | AGGUA |
| | | ATTGT | AUUGU |
| | | GCCTG | GCCUG |
| | Ex8 STOP1 | AGGTG | AGGUG |
| | (Pos 8) | ACCAA | ACCAA |
| | | GATAT | GAUAU |
| | | CAACG | CAACG |
| | Ex8 STOP2 | GGTCG | GGUCG |
| | (Pos 4) | GATCA | GAUCA |
| | | AGTTT | AGUUU |
| | | TCCAC | UCCAC |
| ODC1 | Ex2 SA1 | TTATC | UUAUC |
| | (Pos 9) | ATCCT | AUCCU |
| | | GAAAC | GAAAC |
| | | AAGAG | AAGAG |
| | Ex3 SA1 | TTCAG | UUCAG |
| | (Pos 7) | TCTGA | UCUGA |
| | | AAAAG | AAAAG |
| | | AAGAG | AAGAG |
| | Ex7 STOP1 | AAGGA | AAGGA |
| | (Pos 7) | ACAGA | ACAGA |
| | | CGGGC | CGGGC |
| | | TCTGA | UCUGA |
| | Ex8 SD1 | GAAAT | GAAAU |
| | (Pos 8) | TACCT | UACCU |
| | | TTTGC | UUUGC |
| | | AGAAG | AGAAG |
| | Ex8 SD2 | AGAAA | AGAAA |
| | (Pos 9) | TTACC | UUACC |
| | | TTTTG | UUUUG |
| | | CAGAA | CAGAA |
| | Ex10 SA1 | GTTGC | GUUGC |
| | (Pos 6) | CTGAG | CUGAG |
| | | AAAGA | AAAGA |
| | | AAAAG | AAAAG |
| | Ex10 STOP1 | CTCTC | CUCUC |
| | (Pos 6) | CCAGG | CCAGG |
| | | CACAA | CACAA |
| | | GACAC | GACAC |
| OTULINL | Exon 1 ATG | CCGCC | CCGCC |
| (FAM105A) | | ATGCC | AUGCC |
| | | GGCCG | GGCCG |
| | | CGCTG | CGCUG |
| | Exon 2 STOP | GAAGT | GAAGU |
| | | GACCA | GACCA |
| | | AGTTC | AGUUC |
| | | ACTCC | ACUCC |
| | Exon 3 SA 2 | CAATC | CAAUC |
| | | CACCT | CACCU |
| | | GAAAG | GAAAG |
| | | ATAAA | AUAAA |
| | Exon 3 SA 1 | AATCC | AAUCC |
| | | ACCTG | ACCUG |
| | | AAAGA | AAAGA |
| | | TAAAA | UAAAA |
| | Exon 4 STOP | GTTAT | GUUAU |
| | | TTCAG | UUCAG |
| | | ATATT | AUAUU |
| | | CAGCC | CAGCC |
| | Exon 5 STOP 1 | TGTTT | UGUUU |
| | | TCACA | UCACA |
| | | AGGTT | AGGUU |
| | | GTAAT | GUAAU |
| | Exon 5 STOP 2 | TGGAT | UGGAU |
| | | TCAGC | UCAGC |
| | | AGTAC | AGUAC |
| | | AGTTT | AGUUU |
| | Exon 5 STOP 3 | AAAAC | AAAAC |
| | | ACAGG | ACAGG |
| | | TAAGT | UAAGU |
| | | GTTTG | GUUUG |
| | Exon 5 STOP 4 | AAACA | AAACA |
| | | CAGGT | CAGGU |
| | | AAGTG | AAGUG |
| | | TTTGC | UUUGC |
| | Exon 5 STOP 5 | AACAC | AACAC |
| | | AGGTA | AGGUA |
| | | AGTGT | AGUGU |
| | | TTGCG | UUGCG |
| | Exon 6 STOP 1 | TGAAC | UGAAC |
| | | AAATG | AAAUG |
| | | AAGAC | AAGAC |
| | | TAAAA | UAAAA |
| | Exon 6 STOP 2 | ACTAG | ACUAG |
| | | AGCAG | AGCAG |
| | | GTAAC | GUAAC |
| | | CGGGG | CGGGG |
| | Exon 7 STOP | ATCTC | AUCUC |
| | | CGGCC | CGGCC |
| | | AGTCC | AGUCC |
| | | CTGAG | CUGAG |
| PAG1 | E1 STOP1 | GACAG | GACAG |
| | (Pops 9) | ATGCA | AUGCA |
| | | GATCA | GAUCA |
| | | CCCTG | CCCUG |
| | Ex4 STOP1 | AGGAA | AGGAA |
| | (pos 9) | GTCCA | GUCCA |
| | | GACAT | GACAU |
| | | CGGCC | CGGCC |
| | Ex4 STOP2 | TGGAT | UGGAU |
| | (Pos 9) | TCCCA | UCCCA |
| | | GGACA | GGACA |
| | | GCACA | GCACA |
| | Ex4 SD1 | GCCCA | GCCCA |
| | (Pos 6) | CCTTG | CCUUG |
| | | TTAGT | UUAGU |
| | | TTCAC | UUCAC |
| | Ex4 STOP4 | AAACC | AAACC |
| | (Pos 8) | TTCAG | UUCAG |
| | | GAGAA | GAGAA |
| | | GGAAG | GGAAG |
| | Ex6 SA1 | GCTGA | GCUGA |
| | (Pos 9) | GATCT | GAUCU |
| | | AGGAG | AGGAG |
| | | ACAAA | ACAAA |
| | Ex6 STOP1 | GATAC | GAUAC |
| | (Pos 6) | AGACT | AGACU |
| | | CTCAA | CUCAA |
| | | CAGAG | CAGAG |
| | Ex6 STOP2 | CCATT | CCAUU |
| | (Pos 6) | CAAGG | CAAGG |
| | | GGACC | GGACC |
| | | CACAG | CACAG |
| | Ex6 STOP3 | GGGGC | GGGGC |
| | (Pos 5) | AGTCG | AGUCG |
| | | CTTAC | CUUAC |
| | | AGTTC | AGUUC |
| PDIA3 | Ex1 SD1 | ACTTA | ACUUA |
| | (Pos 6) | CCCTC | CCCUC |
| | | TGACT | UGACU |
| | | TCATA | UCAUA |
| | Ex6 STOP1 | ACAGA | ACAGA |
| | (Pos 7) | GCAAA | GCAAA |
| | | AAATG | AAAUG |
| | | ACCAG | ACCAG |
| | Ex7 SD1 | TTACC | UUACC |
| | (Pos 7) | TGTTT | UGUUU |
| | | CTCCA | CUCCA |
| | | GTAGT | GUAGU |
| | Ex9 SA1 | ACGCC | ACGCC |
| | (Pos 5) | TACAA | UACAA |
| | | TTGGA | UUGGA |
| | | AAACA | AAACA |
| | Ex9 SA2 | CGCCT | CGCCU |
| | (Pos 4) | ACAAT | ACAAU |
| | | TGGAA | UGGAA |
| | | AACAA | AACAA |
| | Ex9 STOP1 | TTCCT | UUCCU |
| | (Pos 7) | GCAGG | GCAGG |
| | | ATTAC | AUUAC |
| | | TTTGA | UUUGA |
| | Ex11 SA1 | TGCTG | UGCUG |
| | (Pos 8) | AGCTG | AGCUG |
| | | TTAAT | UUAAU |
| | | AAAAC | AAAAC |
| | Ex12 SD1 | TCACT | UCACU |
| | (Pos 8) | TACTT | UACUU |
| | | CATAT | CAUAU |
| | | TTCTT | UUCUU |
| PHD1 | Ex1 STOP1 | CCAGC | CCAGC |
| (EGLN2) | (Pos 8) | CGCAG | CGCAG |
| | | CCCCT | CCCCU |
| | | AAGTC | AAGUC |
| | Ex1 STOP2 | TGGCC | UGGCC |
| | (Pos 5) | GGGCC | GGGCC |
| | | AGGAT | AGGAU |
| | | GGGAG | GGGAG |
| | Ex1 STOP3 | ACGGG | ACGGG |
| | (Pos 6) | CAGCT | CAGCU |
| | | AGTGA | AGUGA |
| | | GCCAG | GCCAG |
| | Ex1 STOP4 | CGGGC | CGGGC |
| | (Pos 5) | AGCTA | AGCUA |
| | | GTGAG | GUGAG |
| | | CCAGA | CCAGA |
| | Ex2 SA1 | GGCCT | GGCCU |
| | (Pos 4) | GGCAG | GGCAG |
| | | GGATG | GGAUG |
| | | GAGGG | GAGGG |
| | Ex2 SA2 | CATGG | CAUGG |
| | (Pos 7) | CCTGG | CCUGG |
| | | CAGGG | CAGGG |
| | | ATGGA | AUGGA |
| | Ex2 SA3 | CCATG | CCAUG |
| | (Pos 8) | GCCTG | GCCUG |
| | | GCAGG | GCAGG |
| | | GATGG | GAUGG |
| | Ex2 STOP1 | TAACG | UAACG |
| | (Pos 8) | TCCCA | UCCCA |
| | | GTTCT | GUUCU |
| | | GATTC | GAUUC |
| | Ex2 STOP2 | GAATC | GAAUC |
| | (Pos 5) | AGAAC | AGAAC |
| | | TGGGA | UGGGA |
| | | CGTTA | CGUUA |
| | Ex3 SA1 | TGCAC | UGCAC |
| | (Pos 6) | CTGGG | CUGGG |
| | | GGCAG | GGCAG |
| | | GCCAA | GCCAA |
| | Ex3 SD1 | TCATA | UCAUA |
| | (Pos 6) | CCTGG | CCUGG |
| | | TGGCA | UGGCA |
| | | TAGGC | UAGGC |
| | Ex3 STOP1 | CCTGC | CCUGC |
| | (Pos 8) | TGCAG | UGCAG |
| | | ATCTT | AUCUU |
| | | CCCTG | CCCUG |
| | Ex4 SA1 | TACCT | UACCU |
| | (Pos 4) | GGAGA | GGAGA |
| | | CCAGG | CCAGG |
| | | GTGGT | GUGGU |
| | Ex4 SA2 | GGCGT | GGCGU |
| | (Pos 8) | ACCTG | ACCUG |
| | | GAGAC | GAGAC |
| | | CAGGG | CAGGG |
| | Ex5 SA1 | CCTGA | CCUGA |
| | (Pos 8) | TGCTG | UGCUG |
| | | GGGGT | GGGGU |
| | | GAGAG | GAGAG |
| | Ex5 SA2 | TCCTG | UCCUG |
| | (Pos 9) | ATGCT | AUGCU |
| | | GGGGG | GGGGG |
| | | TGAGA | UGAGA |
| PHD2 | Ex1 STOP1 | CGGCA | CGGCA |
| (EGLN1) | (Pos 4) | GTACT | GUACU |
| | | GCGAG | GCGAG |
| | | CTGTG | CUGUG |
| | Ex1 STOP1 | CGGAC | CGGAC |
| | (Pos 5) | AGCAG | AGCAG |
| | | ATCGG | AUCGG |
| | | CGACG | CGACG |
| | Ex1 STOP2 | GCTTC | GCUUC |
| | (Pos 8) | TTCCA | UUCCA |
| | | GTCCT | GUCCU |
| | | GACGC | GACGC |
| | Ex3 SD1 | TACTA | UACUA |
| | (Pos 6) | CCTTG | CCUUG |
| | | TAGCA | UAGCA |
| | | TATGC | UAUGC |
| | Ex3 STOP1 | ATACT | AUACU |
| | (Pos 7) | TCGAA | UCGAA |
| | | TTTTT | UUUUU |
| | | CCAGA | CCAGA |
| PHD3 | Ex1 STOP1 | GTCAA | GUCAA |
| (EGLN3) | (Pos 7) | GCAGC | GCAGC |
| | | TGCAC | UGCAC |
| | | TGCAC | UGCAC |
| | Ex1 STOP2 | TCAAG | UCAAG |
| | (Pos 6) | CAGCT | CAGCU |
| | | GCACT | GCACU |
| | | GCACC | GCACC |
| | Ex1 STOP3 | CAAGC | CAAGC |
| | (Pos 5) | AGCTG | AGCUG |
| | | CACTG | CACUG |
| | | CACCG | CACCG |
| | Ex3 SA1 | GTAGC | GUAGC |
| | (Pos 5) | TGAAA | UGAAA |
| | | GACAC | GACAC |
| | | AAAGA | AAAGA |
| | Ex3 SA2 | TAGCT | UAGCU |
| | (Pos 4) | GAAAG | GAAAG |
| | | ACACA | ACACA |
| | | AAGAA | AAGAA |
| | Ex3 SD1 | CTATT | CUAUU |
| | (Pos 7) | ACCTG | ACCUG |
| | | GTTGC | GUUGC |
| | | GTAAG | GUAAG |
| | Ex3 SD2 | TATTA | UAUUA |
| | (Pos 6) | CCTGG | CCUGG |
| | | TTGCG | UUGCG |
| | | TAAGA | UAAGA |
| | Ex3 STOP1 | ATCCT | AUCCU |
| | (Pos 7) | GCGGA | GCGGA |
| | | TATTT | UAUUU |
| | | CCAGA | CCAGA |
| | Ex3 STOP2 | TCCTG | UCCUG |
| | (Pos 6) | CGGAT | CGGAU |
| | | ATTTC | AUUUC |
| | | CAGAG | CAGAG |
| | Ex5 SA1 | TCCCT | UCCCU |
| | (Pos 4) | GGGTT | GGGUU |
| | | GGGGA | GGGGA |
| | | CAGAA | CAGAA |
| PIK3CD | Ex1 SD1 | ATACC | AUACC |
| | (Pos 4) | TGCTT | UGCUU |
| | | GATGG | GAUGG |
| | | TGCTG | UGCUG |
| | Ex1 STOP1 | CCTTG | CCUUG |
| | (Pos 8) | GTCCA | GUCCA |
| | | GAATT | GAAUU |
| | | CCATG | CCAUG |
| | Ex2 SA1 | GCAGC | GCAGC |
| | (Pos 5) | TGGAG | UGGAG |
| | | GGACA | GGACA |
| | | GTCAC | GUCAC |
| | Ex2 STOP1 | GCGGT | GCGGU |
| | (Pos 7) | GCCAC | GCCAC |
| | | AGCAG | AGCAG |
| | | CTGGA | CUGGA |
| | Ex2 STOP2 | CGCGG | CGCGG |
| | (Pos 8) | TGCCA | UGCCA |
| | | CAGCA | CAGCA |
| | | GCTGG | GCUGG |
| | Ex2 STOP3 | GGCCC | GGCCC |
| | (Pos 6) | CCAGG | CCAGG |
| | | TTTGA | UUUGA |
| | | GCCGA | GCCGA |
| | Ex2 STOP4 | AGGCC | AGGCC |
| | (Pos 7) | CCCAG | CCCAG |
| | | GTTTG | GUUUG |
| | | AGCCG | AGCCG |
| | Ex3 SA1 | CTCTC | CUCUC |
| | (Pos 6) | CTGTG | CUGUG |
| | | GGGAG | GGGAG |
| | | GAGGG | GAGGG |
| | Ex3 SA2 | AAGCT | AAGCU |
| | (Pos 9) | CTCCT | CUCCU |
| | | GTGGG | GUGGG |
| | | GAGGA | GAGGA |
| | Ex3 SD1 | CTCAC | CUCAC |
| | (Pos 5) | CTGGA | CUGGA |
| | | ACTGG | ACUGG |
| | | CAGAG | CAGAG |
| | Ex3 SD2 | TCACC | UCACC |
| | (Pos 4) | TGGAA | UGGAA |
| | | CTGGC | CUGGC |
| | | AGAGC | AGAGC |
| | Ex4 SA1 | GATGT | GAUGU |
| | (Pos 7) | ACTGA | ACUGA |
| | | GACGG | GACGG |
| | | GGTGC | GGUGC |
| | Ex4 SA2 | ATGTA | AUGUA |
| | (Pos 6) | CTGAG | CUGAG |
| | | ACGGG | ACGGG |
| | | GTGCA | GUGCA |
| | Ex4 SD1 | TCTCA | UCUCA |
| | (Pos 6) | CCTTC | CCUUC |
| | | TTCGC | UUCGC |
| | | AGGAA | AGGAA |
| | Ex4 SD2 | CTCAC | CUCAC |
| | (Pos 5) | CTTCT | CUUCU |
| | | TCGCA | UCGCA |
| | | GGAAT | GGAAU |
| | Ex5 SA1 | AGGCT | AGGCU |
| | (Pos 4) | GGGGG | GGGGG |
| | | CCGGG | CCGGG |
| | | GAAGC | GAAGC |
| | Ex5 SD1 | CCCCA | CCCCA |
| | (Pos 6) | CCTTC | CCUUC |
| | | ATCCG | AUCCG |
| | | CTCGT | CUCGU |
| | Ex6 SA1 | CACCA | CACCA |
| | (Pos 7) | GCTGT | GCUGU |
| | | AGAAG | AGAAG |
| | | GTGCC | GUGCC |
| | Ex6 SA2 | CCACC | CCACC |
| | (Pos 8) | AGCTG | AGCUG |
| | | TAGAA | UAGAA |
| | | GGTGC | GGUGC |
| | Ex6 STOP1 | GTGCA | GUGCA |
| | (Pos 4) | GGCCG | GGCCG |
| | | GGCTT | GGCUU |
| | | TTCCA | UUCCA |
| | Ex7 SA1 | GTCCT | GUCCU |
| | (Pos 4) | GCAGA | GCAGA |
| | | AGGAC | AGGAC |
| | | AGGGC | AGGGC |
| | Ex7 SA2 | GGCAG | GGCAG |
| | (Pos 8) | TCCTG | UCCUG |
| | | CAGAA | CAGAA |
| | | GGACA | GGACA |
| | Ex7 SA3 | GGGCA | GGGCA |
| | (Pos 9) | GTCCT | GUCCU |
| | | GCAGA | GCAGA |
| | | AGGAC | AGGAC |
| | Ex7 STOP1 | CAAGG | CAAGG |
| | (Pos 8) | ACCAG | ACCAG |
| | | CTTAA | CUUAA |
| | | GACCG | GACCG |
| | Ex7 STOP2 | ACAAG | ACAAG |
| | (Pos 9) | GACCA | GACCA |
| | | GCTTA | GCUUA |
| | | AGACC | AGACC |
| | Ex8 SD1 | CACTG | CACUG |
| | (Pos 7) | ACCTT | ACCUU |
| | | CTCCA | CUCCA |
| | | GGGCG | GGGCG |
| | Ex8 SD2 | CCACT | CCACU |
| | (Pos 8) | GACCT | GACCU |
| | | TCTCC | UCUCC |
| | | AGGGC | AGGGC |
| | Ex9 STOP1 | AGGCG | AGGCG |
| | (Pos 9) | CATCC | CAUCC |
| | | ACAGC | ACAGC |
| | | AGCTC | AGCUC |
| | Ex9 STOP2 | TTTGT | UUUGU |
| | (Pos 8) | TGCAG | UGCAG |
| | | ATCTT | AUCUU |
| | | GGAGC | GGAGC |
| | Ex9 STOP3 | TGTTG | UGUUG |
| | (Pos 6) | CAGAT | CAGAU |
| | | CTTGG | CUUGG |
| | | AGCTG | AGCUG |
| | Ex9 STOP3 | TTGTT | UUGUU |
| | (Pos 7) | GCAGA | GCAGA |
| | | TCTTG | UCUUG |
| | | GAGCT | GAGCU |
| | Ex10 SA1 | AGCTG | AGCUG |
| | (Pos 6) | CTGAG | CUGAG |
| | | GGGTG | GGGUG |
| | | TGGGC | UGGGC |
| | Ex10 SA2 | CTGCA | CUGCA |
| | (Pos 7) | GCTGC | GCUGC |
| | | TGAGG | UGAGG |
| | | GGTGT | GGUGU |
| | Ex10 SA3 | GCTGC | GCUGC |
| | (Pos 8) | AGCTG | AGCUG |
| | | CTGAG | CUGAG |
| | | GGGTG | GGGUG |
| | Ex10 STOP1 | TGTGG | UGUGG |
| | (Pos 8) | CCCAG | CCCAG |
| | | GTGGG | GUGGG |
| | | TGGGG | UGGGG |
| | Ex11 SA1 | AGAGC | AGAGC |
| | (Pos 8) | ATCTG | AUCUG |
| | | GGGGG | GGGGG |
| | | AGCCG | AGCCG |
| | Ex12 SA1 | ATCGT | AUCGU |
| | (Pos 8) | CCCTG | CCCUG |
| | | CAGGG | CAGGG |
| | | AAGGA | AAGGA |
| | Ex12 SD1 | GCTAC | GCUAC |
| | (Pos 5) | CGGAG | CGGAG |
| | | GTGCC | GUGCC |
| | | AGAAA | AGAAA |
| | Ex13 SA1 | GGAGC | GGAGC |
| | (Pos 5) | TGGAA | UGGAA |
| | | GGTGA | GGUGA |
| | | AGGGA | AGGGA |
| | Ex13 SA2 | CGGAG | CGGAG |
| | (Pos 6) | CTGGA | CUGGA |
| | | AGGTG | AGGUG |
| | | AAGGG | AAGGG |
| | Ex13 SA3 | TCTCG | UCUCG |
| | (Pos 9) | GAGCT | GAGCU |
| | | GGAAG | GGAAG |
| | | GTGAA | GUGAA |
| | Ex13 STOP1 | GATGA | GAUGA |
| | (Pos 8) | AGCAG | AGCAG |
| | | GTGAG | GUGAG |
| | | GCCCA | GCCCA |
| | Ex14 SA1 | CCCCT | CCCCU |
| | (Pos 4) | GGTGG | GGUGG |
| | | GCAGA | GCAGA |
| | | TGGGA | UGGGA |
| | Ex14 SA2 | CCCCC | CCCCC |
| | (Pos 5) | TGGTG | UGGUG |
| | | GGCAG | GGCAG |
| | | ATGGG | AUGGG |
| | Ex14 SA3 | CTTCC | CUUCC |
| | (Pos 8) | CCCTG | CCCUG |
| | | GTGGG | GUGGG |
| | | CAGAT | CAGAU |
| | Ex14 SA4 | GCTTC | GCUUC |
| | (Pos 9) | CCCCT | CCCCU |
| | | GGTGG | GGUGG |
| | | GCAGA | GCAGA |
| | Ex14 SD1 | CTCAC | CUCAC |
| | (Pos 5) | CAGAC | CAGAC |
| | | TTCAG | UUCAG |
| | | CCAGC | CCAGC |
| | Ex14 SD2 | TCACC | UCACC |
| | (Pos 4) | AGACT | AGACU |
| | | TCAGC | UCAGC |
| | | CAGCA | CAGCA |
| | Ex15 SA1 | ACGCT | ACGCU |
| | (Pos 4) | GCCAG | GCCAG |
| | | GCCAG | GCCAG |
| | | AGAGC | AGAGC |
| | Ex15 SA2 | CACGC | CACGC |
| | (Pos 5) | TGCCA | UGCCA |
| | | GGCCA | GGCCA |
| | | GAGAG | GAGAG |
| | Ex15 STOP1 | GATCC | GAUCC |
| | (Pos 4) | ACAGG | ACAGG |
| | | GGCTT | GGCUU |
| | | CATCT | CAUCU |
| | Ex16 SA1 | GGTCT | GGUCU |
| | (Pos 4) | GTGCC | GUGCC |
| | | ACCGG | ACCGG |
| | | CCGGT | CCGGU |
| | Ex16 SA2 | AGGTC | AGGUC |
| | (Pos 5) | TGTGC | UGUGC |
| | | CACCG | CACCG |
| | | GCCGG | GCCGG |
| | Ex16 SA3 | CGGAG | CGGAG |
| | (Pos 8) | GTCTG | GUCUG |
| | | TGCCA | UGCCA |
| | | CCGGC | CCGGC |
| | Ex16 STOP1 | CCTGC | CCUGC |
| | (Pos 5) | AGATG | AGAUG |
| | | ATCCA | AUCCA |
| | | GCTCA | GCUCA |
| | Ex17 SA1 | ATCCT | AUCCU |
| | (Pos 4) | AGGCA | AGGCA |
| | | AGGGG | AGGGG |
| | | GAAGA | GAAGA |
| | Ex17 SA2 | CATCC | CAUCC |
| | (Pos 5) | TAGGC | UAGGC |
| | | AAGGG | AAGGG |
| | | GGAAG | GGAAG |
| | Ex18 SD1 | CCTGT | CCUGU |
| | (Pos 7) | ACCTG | ACCUG |
| | | CCCAC | CCCAC |
| | | TCTCT | UCUCU |
| | Ex18 ST0P1 | GAGTG | GAGUG |
| | (Pos 8) | GGCAG | GGCAG |
| | | GTACA | GUACA |
| | | GGGGC | GGGGC |
| | Ex19 SA1 | AACAG | AACAG |
| | (Pos 6) | CTGAG | CUGAG |
| | | GGGAG | GGGAG |
| | | GGGAG | GGGAG |
| | Ex20 SA1 | AACCT | AACCU |
| | (Pos 4) | GCAGG | GCAGG |
| | | TAGGG | UAGGG |
| | | GACAG | GACAG |
| | Ex21 SA1 | GTCCT | GUCCU |
| | (Pos 4) | GCAAA | GCAAA |
| | | CAAAT | CAAAU |
| | | CACAG | CACAG |
| | Ex21 STOP1 | GGTTT | GGUUU |
| | (Pos 7) | TCCAG | UCCAG |
| | | CTCTC | CUCUC |
| | | ACGGA | ACGGA |
| | Ex21 STOP2 | TGGTT | UGGUU |
| | (Pos 8) | TTCCA | UUCCA |
| | | GCTCT | GCUCU |
| | | CACGG | CACGG |
| PIKFYVE | Ex2 STOP1 | GGAGA | GGAGA |
| | (Pos 7) | ACAGC | ACAGC |
| | | AGCCT | AGCCU |
| | | TTGAG | UUGAG |
| | Ex2 STOP2 | CTGGT | CUGGU |
| | (Pos 6) | CCAAC | CCAAC |
| | | TTCCA | UUCCA |
| | | CTCAA | CUCAA |
| | Ex5 STOP1 | CTGGC | CUGGC |
| | (Pos 8) | ATCCA | AUCCA |
| | | GTATT | GUAUU |
| | | GTTTC | GUUUC |
| | Ex7 SA1 | AATCA | AAUCA |
| | (Pos 8) | AACTA | AACUA |
| | | TAAAG | UAAAG |
| | | AAAAT | AAAAU |
| | Ex7 SD1 | TCAGT | UCAGU |
| | (Pos 9) | TTACC | UUACC |
| | | TATTT | UAUUU |
| | | CGAGC | CGAGC |
| | Ex9 STOP1 | GTGTG | GUGUG |
| | (Pos 6) | CAGTT | CAGUU |
| | | AAAAG | AAAAG |
| | | ACCTG | ACCUG |
| | Ex10 STOP1 | AGGGC | AGGGC |
| | (Pos 7) | ACAAG | ACAAG |
| | | CTATA | CUAUA |
| | | GCAAT | GCAAU |
| | Ex11 SA1 | TACTC | UACUC |
| | (Pos 5) | TGAAA | UGAAA |
| | | GGATG | GGAUG |
| | | AAGAC | AAGAC |
| | Ex11 STOP1 | ACAGA | ACAGA |
| | (Pos 7) | ACAGA | ACAGA |
| | | TAGCT | UAGCU |
| | | GAAGA | GAAGA |
| | Ex12 SA1 | AATCT | AAUCU |
| | (Pos 4) | TTTAG | UUUAG |
| | | TGTTG | UGUUG |
| | | GGAAG | GGAAG |
| | Ex12 SA2 | GAATC | GAAUC |
| | (Pos 5) | TTTTA | UUUUA |
| | | GTGTT | GUGUU |
| | | GGGAA | GGGAA |
| | Ex12 SA3 | AGAAT | AGAAU |
| | (Pos 6) | CTTTT | CUUUU |
| | | AGTGT | AGUGU |
| | | TGGGA | UGGGA |
| | Ex12 SD1 | CTCCT | CUCCU |
| | (Pos 7) | ACCTT | ACCUU |
| | | TTTGG | UUUGG |
| | | TCAGC | UCAGC |
| | Ex12 SD2 | TCCTA | UCCUA |
| | (Pos 6) | CCTTT | CCUUU |
| | | TTGGT | UUGGU |
| | | CAGCA | CAGCA |
| | Ex14 SD1 | GCCTT | GCCUU |
| | (Pos 7) | ACAGC | ACAGC |
| | | AACCT | AACCU |
| | | CTCCA | CUCCA |
| | Ex17 SD1 | ATTCT | AUUCU |
| | (Pos 8) | TACCT | UACCU |
| | | GAAGC | GAAGC |
| | | ACAAT | ACAAU |
| PPARa | Ex2 SA1 | AAGCG | AAGCG |
| | (Pos 9) | TGTCT | UGUCU |
| | | GGGGA | GGGGA |
| | | AAAAG | AAAAG |
| | Ex4 SA1 | GAATC | GAAUC |
| | (Pos 7) | GCTAG | GCUAG |
| | | GGTTT | GGUUU |
| | | GGAGG | GGAGG |
| | Ex4 SA2 | CGAAT | CGAAU |
| | (Pos 8) | CGCTA | CGCUA |
| | | GGGTT | GGGUU |
| | | TGGAG | UGGAG |
| | Ex4 SA3 | ACGAA | ACGAA |
| | (Pos 9) | TCGCT | UCGCU |
| | | AGGGT | AGGGU |
| | | TTGGA | UUGGA |
| | Ex4 SD1 | AACAC | AACAC |
| | (Pos 9) | CTACT | CUACU |
| | | GGATT | GGAUU |
| | | GTTAC | GUUAC |
| | Ex5 STOP1 | TGGCA | UGGCA |
| | (Pos 8) | TCCAG | UCCAG |
| | | AACAA | AACAA |
| | | GGAGG | GGAGG |
| | Ex5 STOP2 | CATCC | CAUCC |
| | (Pos 5) | AGAAC | AGAAC |
| | | AAGGA | AAGGA |
| | | GGCGG | GGCGG |
| | Ex6 STOP1 | CGCTA | CGCUA |
| | (Pos 9) | CTGCA | CUGCA |
| | | GGAGA | GGAGA |
| | | TCTAC | UCUAC |
| | Ex6 STOP2 | GGTGC | GGUGC |
| | (Pos 5) | AGATC | AGAUC |
| | | ATCAA | AUCAA |
| | | GAAGA | GAAGA |
| | Ex6 STOP3 | CCACC | CCACC |
| | (Pos 8) | TGCAG | UGCAG |
| | | AGCAA | AGCAA |
| | | CCACC | CCACC |
| PPARd | Ex1 SD1 | TCACC | UCACC |
| | (Pos 4) | TGTGT | UGUGU |
| | | AGCTG | AGCUG |
| | | CTGGA | CUGGA |
| | Ex1 SD2 | CTCCT | CUCCU |
| | (Pos 8) | CACCT | CACCU |
| | | GTGTA | GUGUA |
| | | GCTGC | GCUGC |
| | Ex1 STOP | GCCAC | GCCAC |
| | (Pos 5) | AGGAG | AGGAG |
| | | GAAGC | GAAGC |
| | | CCCTG | CCCUG |
| | Ex2 SA1 | AGAGG | AGAGG |
| | (Pos 7) | TCTGC | UCUGC |
| | | GGACA | GGACA |
| | | CACGA | CACGA |
| | Ex2 STOP1 | CAACT | CAACU |
| | (Pos 7) | GCAGA | GCAGA |
| | | TGGGC | UGGGC |
| | | TGTGA | UGUGA |
| | Ex2 STOP2 | AACTG | AACUG |
| | (Pos 6) | CAGAT | CAGAU |
| | | GGGCT | GGGCU |
| | | GTGAC | GUGAC |
| | Ex3 SA1 | AGCCC | AGCCC |
| | (Pos 5) | TGAAG | UGAAG |
| | | CACCA | CACCA |
| | | AGAAC | AGAAC |
| | Ex3 STOP1 | CTTCC | CUUCC |
| | (pos 5) | AGAAG | AGAAG |
| | | TGCCT | UGCCU |
| | | GGCAC | GGCAC |
| | Ex4 SA1 | GGATA | GGAUA |
| | (Pos 7) | GCTGC | GCUGC |
| | | ACAGG | ACAGG |
| | | GAAGG | GAAGG |
| | Ex4 SA2 | CGGAT | CGGAU |
| | (Pos 8) | AGCTG | AGCUG |
| | | CACAG | CACAG |
| | | GGAAG | GGAAG |
| | Ex4 SA3 | ACGGA | ACGGA |
| | (Pos 9) | TAGCT | UAGCU |
| | | GCACA | GCACA |
| | | GGGAA | GGGAA |
| | Ex4 SD1 | AACAC | AACAC |
| | (Pos 9) | TCACC | UCACC |
| | | GCCGT | GCCGU |
| | | GTGGC | GUGGC |
| | Ex5 SD1 | CTCAC | CUCAC |
| | (Pos 5) | CTCCA | CUCCA |
| | | CACAG | CACAG |
| | | AATGA | AAUGA |
| | Ex5 STOP1 | GTGGC | GUGGC |
| | (Pos 5) | AGGCA | AGGCA |
| | | GAGAA | GAGAA |
| | | GGGGC | GGGGC |
| | Ex6 SA1 | GGCCG | GGCCG |
| | (Pos 8) | GTCTG | GUCUG |
| | | TGGGG | UGGGG |
| | | ACACA | ACACA |
| | Ex6 SA2 | TGGCC | UGGCC |
| | (Pos 9) | GGTCT | GGUCU |
| | | GTGGG | GUGGG |
| | | GACAC | GACAC |
| | Ex6 SA3 | CAGCT | CAGCU |
| | (Pos 4) | TGGGG | UGGGG |
| | | AAGAG | AAGAG |
| | | GTACT | GUACU |
| | Ex6 SA4 | GCAGC | GCAGC |
| | (Pos 5) | TTGGG | UUGGG |
| | | GAAGA | GAAGA |
| | | GGTAC | GGUAC |
| | Ex6 STOP1 | TCTGC | UCUGC |
| | (Pos 8) | TCCAG | UCCAG |
| | | GAGAT | GAGAU |
| | | CTACA | CUACA |
| PRDMI1 | Iso 1 ATG | GGTCA | GGUCA |
| | | TGGCC | UGGCC |
| | | GCCAG | GCCAG |
| | | ACCCT | ACCCU |
| | Exon 2 STOP | GTCCA | GUCCA |
| | | GTGTC | GUGUC |
| | | CCAGA | CCAGA |
| | | ATGCC | AUGCC |
| | Exon 2 SD | AATCA | AAUCA |
| | | CCTCT | CCUCU |
| | | GAACA | GAACA |
| | | ATCCC | AUCCC |
| | Exon 3 SA | CAGCC | CAGCC |
| | | TGGAA | UGGAA |
| | | GAGAA | GAGAA |
| | | AGGAA | AGGAA |
| | Exon 3 SD 1 | GCTTA | GCUUA |
| | | CCTCT | CCUCU |
| | | TCACT | UCACU |
| | | GTTGG | GUUGG |
| | Exon 3 SD 2 | GAGGC | GAGGC |
| | | TTACC | UUACC |
| | | TCTTC | UCUUC |
| | | ACTGT | ACUGU |
| | Exon 6 SA 3 | TTGTG | UUGUG |
| | | CTGAA | CUGAA |
| | | ATAAA | AUAAA |
| | | GAAAA | GAAAA |
| | Exon 6 SA 2 | TGTGC | UGUGC |
| | | TGAAA | UGAAA |
| | | TAAAG | UAAAG |
| | | AAAAA | AAAAA |
| | Exon 6 SA 1 | GTGCT | GUGCU |
| | | GAAAT | GAAAU |
| | | AAAGA | AAAGA |
| | | AAAAG | AAAAG |
| | Exon 6 SD | CTACC | CUACC |
| | | TTCAG | UUCAG |
| | | ATTGG | AUUGG |
| | | AGAGC | AGAGC |
| | Exon 7 SD | CTGCG | CUGCG |
| | | CACCT | CACCU |
| | | GGCAT | GGCAU |
| | | TCATG | UCAUG |
| | Exon 8 STOP | TTGCA | UUGCA |
| | | AAGAA | AAGAA |
| | | ACATG | ACAUG |
| | | GGGAA | GGGAA |
| PRKACA | Ex1 Isoform SD1 | TGACC | UGACC |
| | (Pos 4) | GACAT | GACAU |
| | | TCCAT | UCCAU |
| | | GGCCA | GGCCA |
| | Ex2 SA1 | TTTCA | UUUCA |
| | (Pos 6) | CTGAA | CUGAA |
| | | AGGGA | AGGGA |
| | | GAGAG | GAGAG |
| | Ex2 SA2 | CTTTC | CUUUC |
| | (Pos 7) | ACTGA | ACUGA |
| | | AAGGG | AAGGG |
| | | AGAGA | AGAGA |
| | Ex2 SA3 | TCTTT | UCUUU |
| | (Pos 8) | CACTG | CACUG |
| | | AAAGG | AAAGG |
| | | GAGAG | GAGAG |
| | Ex3 SA1 | TGTTC | UGUUC |
| | (Pos5) | TGTGG | UGUGG |
| | | GCAGA | GCAGA |
| | | GGGGT | GGGGU |
| | Ex3 SA2 | GCTGT | GCUGU |
| | (Pos 9) | GTTCT | GUUCU |
| | | GTGGG | GUGGG |
| | | CAGAG | CAGAG |
| | Ex3 SD1 | ACCTC | ACCUC |
| | (Pos 7) | ACCTT | ACCUU |
| | | CTGTT | CUGUU |
| | | TGTCG | UGUCG |
| | Ex4 SA1 | CCACC | CCACC |
| | (Pos 5) | TGGGA | UGGGA |
| | | AGGGA | AGGGA |
| | | AGGAG | AGGAG |
| | Ex4 SA2 | ACCAC | ACCAC |
| | (Pos 6) | CTGGG | CUGGG |
| | | AAGGG | AAGGG |
| | | AAGGA | AAGGA |
| | Ex4 SA3 | CACCA | CACCA |
| | (Pos 7) | CCTGG | CCUGG |
| | | GAAGG | GAAGG |
| | | GAAGG | GAAGG |
| | Ex5 SA1 | GTCCT | GUCCU |
| | (Pos 4) | GTGGG | GUGGG |
| | | AAGCA | AAGCA |
| | | GTGGC | GUGGC |
| | Ex5 SA2 | AGTTG | AGUUG |
| | (Pos 8) | TCCTG | UCCUG |
| | | TGGGA | UGGGA |
| | | AGCAG | AGCAG |
| | Ex6 SA1 | CTCAC | CUCAC |
| | (Pos 5) | TGATG | UGAUG |
| | | GGGAC | GGGAC |
| | | AAATG | AAAUG |
| | Ex6 SA2 | GCTCA | GCUCA |
| | (Pos 6) | CTGAT | CUGAU |
| | | GGGGA | GGGGA |
| | | CAAAT | CAAAU |
| | Ex6 SA3 | GGCTC | GGCUC |
| | (Pos 7) | ACTGA | ACUGA |
| | | TGGGG | UGGGG |
| | | ACAAA | ACAAA |
| | Ex6 SD1 | GCACC | GCACC |
| | (Pos 4) | TGAAT | UGAAU |
| | | GTAGC | GUAGC |
| | | CCTGC | CCUGC |
| | Ex7 SA1 | TCACC | UCACC |
| | (Pos 7) | TGTGG | UGUGG |
| | | GCACA | GCACA |
| | | AGAAC | AGAAC |
| | Ex8 SA1 | AGCCC | AGCCC |
| | (Pos 5) | TGGAG | UGGAG |
| | | CAAGA | CAAGA |
| | | TGGGG | UGGGG |
| | Ex8 SA2 | TAGCC | UAGCC |
| | (Pos 6) | CTGGA | CUGGA |
| | | GCAAG | GCAAG |
| | | ATGGG | AUGGG |
| | Ex8 SA3 | GTAGC | GUAGC |
| | (Pos 7) | CCTGG | CCUGG |
| | | AGCAA | AGCAA |
| | | GATGG | GAUGG |
| | Ex8 SA4 | TGTAG | UGUAG |
| | (Pos 8) | CCCTG | CCCUG |
| | | GAGCA | GAGCA |
| | | AGATG | AGAUG |
| | Ex8 SA5 | TTGTA | UUGUA |
| | (Pos 9) | GCCCT | GCCCU |
| | | GGAGC | GGAGC |
| | | AAGAT | AAGAU |
| | Ex9 SA1 | CACCT | CACCU |
| | (Pos 4) | GGAGG | GGAGG |
| | | AAGGG | AAGGG |
| | | GTACA | GUACA |
| | Ex9 SD1 | GCCCA | GCCCA |
| | (Pos 6) | CCTTC | CCUUC |
| | | CTCTG | CUCUG |
| | | GTAGA | GUAGA |
| | Ex1 STOP1 | GGAGC | GGAGC |
| | (Pos 5) | GGGGG | GGGGG |
| | | GGAGA | GGAGA |
| | | AGCGG | AGCGG |
| | Ex1 STOP2 | AAACA | AAACA |
| | (Pos 4) | AAAGG | AAAGG |
| | | AGATA | AGAUA |
| | | TCAAG | UCAAG |
| PTEN | Ex2 | ATATC | AUAUC |
| | (SA1 | TGAGT | UGAGU |
| | (Pos 5) | ACTTT | ACUUU |
| | | AGTTA | AGUUA |
| | Ex4 SD1 | CCTAC | CCUAC |
| | (Pos 5) | CTCTG | CUCUG |
| | | CAATT | CAAUU |
| | | AAATT | AAAUU |
| | Ex5 SD1 | ATAAC | AUAAC |
| | (Pos 9) | TTACC | UUACC |
| | | TTTTT | UUUUU |
| | | GTCTC | GUCUC |
| | Ex1 STOP1 pos 8) | TCGCT | UCGCU |
| | | GGCAG | GGCAG |
| | | CCGCT | CCGCU |
| | | GTACT | GUACU |
| PTPN2 | Ex6 SA1 | ACTCT | ACUCU |
| | (Pos 4) | AAAAA | AAAAA |
| | | GTGAA | GUGAA |
| | | AATCA | AAUCA |
| | Ex9 STOP1 | AGGTG | AGGUG |
| | (pos 6) | CAGCA | CAGCA |
| | | GATGA | GAUGA |
| | | AACAG | AACAG |
| | Ex2 SA1 | ACCAC | ACCAC |
| | (Pos 6) | CTGGG | CUGGG |
| | | CGGCC | CGGCC |
| | | CAGGC | CAGGC |
| | Ex2 SA2 | CCACC | CCACC |
| | (Pos 5) | TGGGC | UGGGC |
| | | GGCCC | GGCCC |
| | | AGGCA | AGGCA |
| | Ex3 SA1 | CCCCC | CCCCC |
| | (Pos 9) | ACCCT | ACCCU |
| | | GCAGG | GCAGG |
| | | GCACC | GCACC |
| | Ex3 SA2 | CCACC | CCACC |
| | (Pos 6) | CTGCA | CUGCA |
| | | GGGCA | GGGCA |
| | | CCAGG | CCAGG |
| | Ex3 SD1 | AGCCC | AGCCC |
| | (pos 9) | TCACC | UCACC |
| | | TCTCA | UCUCA |
| | | CTAGT | CUAGU |
| | Ex4 SA1 | CACCT | CACCU |
| | (Pos 4) | AGAGA | AGAGA |
| | | AGGCA | AGGCA |
| | | GCGTC | GCGUC |
| | Ex5 SA1 | ACCCT | ACCCU |
| | (Pos 4) | GGGGG | GGGGG |
| | | GAGCC | GAGCC |
| | | AAATT | AAAUU |
| | Ex7 SA1 | CAAAC | CAAAC |
| | (Pos 7) | TCTGA | UCUGA |
| | | GATGT | GAUGU |
| | | GGGTG | GGGUG |
| | Ex7 SA1 | GCAAA | GCAAA |
| | (Pos 8) | CTCTG | CUCUG |
| | | AGATG | AGAUG |
| | | TGGGT | UGGGU |
| | Ex8 SA1 | TCAAC | UCAAC |
| | (Pos 5) | TGGGA | UGGGA |
| | | GTGGG | GUGGG |
| | | CGGAG | CGGAG |
| | Ex8 SD1 | ACTGC | ACUGC |
| | (Pos 9) | TGACC | UGACC |
| | | TTGAT | UUGAU |
| | | GTAGT | GUAGU |
| | Ex9 SA1 | GCTGG | GCUGG |
| | (Pos 8) | TTCTG | UUCUG |
| | | GACGC | GACGC |
| | | AAGCG | AAGCG |
| | Ex10 SA1 | TTGTT | UUGUU |
| | (Pos 6) | CTGGA | CUGGA |
| | | AAGGG | AAGGG |
| | | AGGGT | AGGGU |
| PTPN6 | Ex10 SA2 | TGTTC | UGUUC |
| | (Pos 5) | TGGAA | UGGAA |
| | | AGGGA | AGGGA |
| | | GGGTC | GGGUC |
| | Ex10 SD1 | GCCAC | GCCAC |
| | (Pos 9) | TCACA | UCACA |
| | | TTGTC | UUGUC |
| | | CAGCG | CAGCG |
| | Ex11 SA1 | CTCCC | CUCCC |
| | (Pos 5) | TAAGC | UAAGC |
| | | CGAGG | CGAGG |
| | | ACATA | ACAUA |
| | Ex11 SA2 | TCTCC | UCUCC |
| | (Pos 6) | CTAAG | CUAAG |
| | | CCGAG | CCGAG |
| | | GACAT | GACAU |
| | Ex11 SD1 | TCACC | UCACC |
| | (Pos 4) | TGCAG | UGCAG |
| | | TGCAC | UGCAC |
| | | GATGA | GAUGA |
| | Ex12 SA1 | CCGGC | CCGGC |
| | (Pos 7) | GCTGG | GCUGG |
| | | GGAAA | GGAAA |
| | | GACGG | GACGG |
| | Ex12 SA2 | GCCGG | GCCGG |
| | (Pos 8) | CGCTG | CGCUG |
| | | GGGAA | GGGAA |
| | | AGACG | AGACG |
| | Ex12 SA3 | TGCCG | UGCCG |
| | (Pos 9) | GCGCT | GCGCU |
| | | GGGGA | GGGGA |
| | | AAGAC | AAGAC |
| | Ex14 SD1 | CACTC | CACUC |
| | (Pos 7) | ACTTG | ACUUG |
| | | GACGA | GACGA |
| | | GGTGC | GGUGC |
| | Ex14 SD2 | CCACT | CCACU |
| | (Pos 8) | CACTT | CACUU |
| | | GGACG | GGACG |
| | | AGGTG | AGGUG |
| | Ex15 SA1 | TGTCT | UGUCU |
| | (Pos 4) | GCAGC | GCAGC |
| | | CGGGT | CGGGU |
| | | GCAGG | GCAGG |
| | Ex15 SA2 | GTGTC | GUGUC |
| | (Pos 5) | TGCAG | UGCAG |
| | | CCGGG | CCGGG |
| | | TGCAG | UGCAG |
| | Ex15 SA3 | TGTGT | UGUGU |
| | (Pos 6) | CTGCA | CUGCA |
| | | GCCGG | GCCGG |
| | | GTGCA | GUGCA |
| | Ex15 SD1 | CACCG | CACCG |
| | (Pos 6) | CTCAC | CUCAC |
| | | TTCCT | UUCCU |
| | | CTTGA | CUUGA |
| | Ex15 SD2 | GCACC | GCACC |
| | (Pos 7) | GCTCA | GCUCA |
| | | CTTCC | CUUCC |
| | | TCTTG | UCUUG |
| | Ex3 SA1 | TTTCT | UUUCU |
| | (Pos 7) | TCTAA | UCUAA |
| | | AATAG | AAUAG |
| | | TCCAT | UCCAU |
| PTPN11 | Ex3 SD1 | GTTAC | GUUAC |
| | (Pos 9) | TGACC | UGACC |
| | | TTTCA | UUUCA |
| | | GAGGT | GAGGU |
| | Ex13 SD1 | ACTAC | ACUAC |
| | (Pos 9) | TTACT | UUACU |
| | | CTGCA | CUGCA |
| | | CAGGG | CAGGG |
| RASA2 | Ex2 SA1 | GACCT | GACCU |
| | (Pos 4) | AAAAT | AAAAU |
| | | ATAAA | AUAAA |
| | | AAATT | AAAUU |
| | Ex5 SD1 | ATTTA | AUUUA |
| | (pos 6) | CCTGA | CCUGA |
| | | ACCTC | ACCUC |
| | | TGAAT | UGAAU |
| | Ex6 SD1 | CTTAC | CUUAC |
| | (Pos 5) | TGTAC | UGUAC |
| | | AACAA | AACAA |
| | | GCTGC | GCUGC |
| | Ex10 SA1 | CGATC | CGAUC |
| | (Pos 6) | CTGAA | CUGAA |
| | | AAUGA | AAUUG |
| | | AAAC | AAAAC |
| | Ex10 SD1 | CCCUA | CCCUU |
| | (Pos 7) | CCAGG | ACCAG |
| | | CUGAT | GCUUG |
| | | GAG | AUGAG |
| | Ex12 SA1 | GATAU | GAUAU |
| | (Pos 9) | GGCTA | UGGCU |
| | | AATAC | AAAUA |
| | | AGAA | CAGAA |
| | Ex13 SD1 | UCTGT | UUCUG |
| | (Pos 8) | ACCTC | UACCU |
| | | ATCAA | CAUCA |
| | | GAAT | AGAAU |
| | Ex15 SA1 | UCTCC | UUCUC |
| | (Pos 6) | TGCAG | CUGCA |
| | | GATUA | GGAUU |
| | | AAA | UAAAA |
| | Ex16 SA1 | GGTCA | GGUCA |
| | (Pos 7) | TCTGC | UCUGC |
| | | AGGAA | AGGAA |
| | | AAAAA | AAAAA |
| | Ex19 SA1 | CAAGA | CAAGA |
| | (Pos 7) | ACTAA | ACUAA |
| | | ATGGG | AUGGG |
| | | GAAAT | GAAAU |
| SIGLEC15 | Ex3 SA1 | GGCGA | GGCGA |
| | (Pos 8) | GCCTG | GCCUG |
| | | AGGGC | AGGGC |
| | | GGGGC | GGGGC |
| | Ex3 SA2 | TGGCG | UGGCG |
| | (Pos 9) | AGCCT | AGCCU |
| | | GAGGG | GAGGG |
| | | CGGGG | CGGGG |
| | Ex3 SD1 | CCTCG | CCUCG |
| | (Pos 6) | CCTGT | CCUGU |
| | | CACGT | CACGU |
| | | GCAGC | GCAGC |
| | Ex3 STOP1 | GCGCG | GCGCG |
| | (pos 6) | CCAGA | CCAGA |
| | | TGGCC | UGGCC |
| | | GTCAG | GUCAG |
| | Ex3 STOP2 | TGCAT | UGCAU |
| | (Pos 9) | GGACC | GGACC |
| | | AGCGC | AGCGC |
| | | TGCGC | UGCGC |
| | Ex3 STOP3 | GTCCA | GUCCA |
| | (Pos 8) | TGCAG | UGCAG |
| | | GTGCC | GUGCC |
| | | ACCCG | ACCCG |
| | Ex3 STOP4 | AGCGC | AGCGC |
| | (Pos 5) | AGCGC | AGCGC |
| | | TGGTC | UGGUC |
| | | CATGC | CAUGC |
| | Ex4 SD | CGCAC | CGCAC |
| | (Pos 9) | CCACC | CCACC |
| | | TGGGC | UGGGC |
| | | GGCGG | GGCGG |
| | Ex4 SD1 | CGCGG | CGCGG |
| | (Pos 6) | CTGCA | CUGCA |
| | | GGGGA | GGGGA |
| | | GAAGG | GAAGG |
| | Ex4 SD1 | GCACC | GCACC |
| | (Pos 8) | CACCT | CACCU |
| | | GGGCG | GGGCG |
| | | GCGGC | GCGGC |
| | Ex4 SD1 | CGGCG | CGGCG |
| | (Pos 9) | CGGCT | CGGCU |
| | | GCAGG | GCAGG |
| | | GGAGA | GGAGA |
| | Ex4 SD3 | GCGGC | GCGGC |
| | (Pos 5) | TGCAG | UGCAG |
| | | GGGAG | GGGAG |
| | | AAGGC | AAGGC |
| | Ex4 STOP1 | GGGCC | GGGCC |
| | (Pos 9) | GGACC | GGACC |
| | | AGGCG | AGGCG |
| | | AGGGC | AGGGC |
| | Ex4 STOP2 | CCGGA | CCGGA |
| | (Pos 6) | CCAGG | CCAGG |
| | | CGAGG | CGAGG |
| | | GCGGG | GCGGG |
| | Ex6 STOP1 | ATUGA | AUUUG |
| | (Pos 9) | GCCAG | AGCCA |
| | | ATGAA | GAUGA |
| | | CCCC | ACCCC |
| SLA | Ex2 SD1 | UACCC | UUACC |
| | (pos 4) | TCCGG | CUCCG |
| | | GUGGG | GGUUG |
| | | CAG | GGCAG |
| | Ex2 SD2 | CTTAC | CUUAC |
| | (Pos 5) | CCTCC | CCUCC |
| | | GGGUG | GGGUU |
| | | GGCA | GGGCA |
| | Ex3 SA1 | GTCCT | GUCCU |
| | (Pos 4)) | GGGGA | GGGGA |
| | | AACAA | AACAA |
| | | AGGCA | AGGCA |
| | Ex3 SA2 | ATCCA | AUCCA |
| | (pos 9) | GTCCT | GUCCU |
| | | GGGGA | GGGGA |
| | | AACAA | AACAA |
| | Ex4 SD1 | TACTC | UACUC |
| | (Pos 7) | ACCCA | ACCCA |
| | | TGGTA | UGGUA |
| | | AACTC | AACUC |
| | Ex6 SA1 | TAAAA | UAAAA |
| | (Pos 8) | CCCTG | CCCUG |
| | | CAGGA | CAGGA |
| | | GGTGG | GGUGG |
| | Ex8 SA1 | AGTCT | AGUCU |
| | (Pos 4) | GTGGG | GUGGG |
| | | CCAGA | CCAGA |
| | | AGAAA | AGAAA |
| | Ex1 SD1 | ACTCA | ACUCA |
| | (Pos 6) | CCTGT | CCUGU |
| | | GAGCT | GAGCU |
| | | GCCAA | GCCAA |
| SLAMF7 | Ex1 STOP1 | GCTGC | GCUGC |
| | (Pos 5) | CAAAG | CAAAG |
| | | GATAT | GAUAU |
| | | AGATG | AGAUG |
| | Ex1 STOP2 | CTGCC | CUGCC |
| | (Pos 4) | AAAGG | AAAGG |
| | | ATATA | AUAUA |
| | | GATGA | GAUGA |
| | Ex3 SD1 | GTCAC | GUCAC |
| | (Pos 5) | CUCAC | CUUCA |
| | | AGAGC | CAGAG |
| | | UCC | CUUCC |
| | Ex3 SD2 | CAGCA | CAGCA |
| | (Pos 7) | CCUCA | CCUUC |
| | | GAGAA | AGAGA |
| | | TGGG | AUGGG |
| | Ex3 SD3 | CACCU | CACCU |
| | (Pos 4) | CAGAG | UCAGA |
| | | AATGG | GAAUG |
| | | GTGG | GGUGG |
| | Ex4 SD1 | ATGTA | AUGUA |
| | (Pos 8) | CTCTA | CUCUA |
| | | AAAGC | AAAGC |
| | | AAGU | AAGUU |
| | Ex4 SD2 | AATGT | AAUGU |
| | (Pos 9) | ACTCT | ACUCU |
| | | AAAAG | AAAAG |
| | | CAAGT | CAAGU |
| SOCS1 | Ex1 STOP1 | CGCTG | CGCUG |
| | (Pos 9) | CGCCA | CGCCA |
| | | GCGCC | GCGCC |
| | | GCGTG | GCGUG |
| | Ex1 STOP2 | GGGCC | GGGCC |
| | (Pos 7) | CCCAG | CCCAG |
| | | TAGAA | UAGAA |
| | | TCCGC | UCCGC |
| STK4 | Ex1 SD1 | CTTAC | CUUAC |
| | (Pos 9) | CTACC | CUACC |
| | | TCCCA | UCCCA |
| | | ATGTC | AUGUC |
| | Ex1 SA2 | CTGCA | CUGCA |
| | (Pos 7) | TCTAC | UCUAC |
| | | AGTAA | AGUAA |
| | | TCTGA | UCUGA |
| | Ex5 SA1 | ATGGT | AUGGU |
| | (Pos 9) | ATCCT | AUCCU |
| | | AAAAT | AAAAU |
| | | AGAAA | AGAAA |
| | Ex6 SD1 | TCATA | UCAUA |
| | (Pos 6) | CCTGC | CCUGC |
| | | AGGAG | AGGAG |
| | | CTGAG | CUGAG |
| | Ex9 SA1 | TAAGC | UAAGC |
| | (Pos 5) | TAGAA | UAGAA |
| | | GAGAA | GAGAA |
| | | GTGGA | GUGGA |
| | Ex9 SA2 | CTCTT | CUCUU |
| | (Pos 9) | AAGCT | AAGCU |
| | | AGAAG | AGAAG |
| | | AGAAG | AGAAG |
| SUV39H1 | Ex2 SA1 | GCTGC | GCUGC |
| | (Pos 9) | AGCCT | AGCCU |
| | | GGATC | GGAUC |
| | | AAGCG | AAGCG |
| | Ex2 STOP1 | GCTGC | GCUGC |
| | (Pos 5) | AGGAC | AGGAC |
| | | CTGTG | CUGUG |
| | | CCGCC | CCGCC |
| | Ex3 SA1 | TTCCT | UUCCU |
| | (Pos 4) | GTTGG | GUUGG |
| | | GGGTG | GGGUG |
| | | GGTAG | GGUAG |
| | Ex3 SA2 | GTTCC | GUUCC |
| | (Pos 5) | TGTTG | UGUUG |
| | | GGGGT | GGGGU |
| | | GGGTA | GGGUA |
| | Ex3 SA3 | TGTTC | UGUUC |
| | (Pos 5) | CTGTT | CUGUU |
| | | GGGGG | GGGGG |
| | | TGGGT | UGGGU |
| | Ex3 STOP1 | ACAGG | ACAGG |
| | (Pos8) | AACAG | AACAG |
| | | GAATA | GAAUA |
| | | TTACC | UUACC |
| | Ex3 STOP2 | GATAT | GAUAU |
| | (Pos 6) | CCACG | CCACG |
| | | CCATT | CCAUU |
| | | TCACC | UCACC |
| TMEM222 | Ex1 SD1 | ACTCA | ACUCA |
| | (Pos 6) | CGTGA | CGUGA |
| | | GCACC | GCACC |
| | | GGGAT | GGGAU |
| | Ex1 SD2 | GACTC | GACUC |
| | (Pos 7) | ACGTG | ACGUG |
| | | AGCAC | AGCAC |
| | | CGGGA | CGGGA |
| | Ex2 SA1 | CACCT | CACCU |
| | (Pos 4) | GTGAG | GUGAG |
| | | GAAAA | GAAAA |
| | | GGACG | GGACG |
| | Ex2 SA2 | CCACC | CCACC |
| | (Pos 5) | TGTGA | UGUGA |
| | | GGAAA | GGAAA |
| | | AGGAC | AGGAC |
| | Ex2 SD1 | GACTC | GACUC |
| | (Pos 7) | ACTGA | ACUGA |
| | | GACAA | GACAA |
| | | AGTAG | AGUAG |
| | Ex2 SA3 | ACCAC | ACCAC |
| | (Pos 6) | CTGTG | CUGUG |
| | | AGGAA | AGGAA |
| | | AAGGA | AAGGA |
| | Ex2 SD2 | GGACT | GGACU |
| | (Pos 8) | CACTG | CACUG |
| | | AGACA | AGACA |
| | | AAGTA | AAGUA |
| | Ex2 SD3 | GGGAC | GGGAC |
| | (Pos 9) | TCACT | UCACU |
| | | GAGAC | GAGAC |
| | | AAAGT | AAAGU |
| | Ex3 SD1 | TTACT | UUACU |
| | (Pos 4) | TGGCA | UGGCA |
| | | GGCTT | GGCUU |
| | | TCCAA | UCCAA |
| | Ex4 SA1 | CAGTA | CAGUA |
| | (Pos 6) | CCTGG | CCUGG |
| | | GGGGA | GGGGA |
| | | GAGAA | GAGAA |
| | Ex4 SA1 | CCAGT | CCAGU |
| | (Pos 7) | ACCTG | ACCUG |
| | | GGGGG | GGGGG |
| | | AGAGA | AGAGA |
| | Ex5 SA1 | TTGTG | UUGUG |
| | (Pos 6) | CTGGA | CUGGA |
| | | GGCAC | GGCAC |
| | | CAGAA | CAGAA |
| | Ex6 SA2 | ATTGT | AUUGU |
| | (Pos 7) | GCTGG | GCUGG |
| | | AGGCA | AGGCA |
| | | CCAGA | CCAGA |
| | Ex7 SA1 | CCCAA | CCCAA |
| | (Pos 8) | CGCTG | CGCUG |
| | | ACAGA | ACAGA |
| | | GAGAA | GAGAA |
| TNFAIP3 | Ex2 SA1 | GTCAC | GUCAC |
| | (Pos 6) | CTGAG | CUGAG |
| | | GACAG | GACAG |
| | | AAAGG | AAAGG |
| | Ex2 SA2 | GCCGT | GCCGU |
| | (Pos 9) | CACCT | CACCU |
| | | GAGGA | GAGGA |
| | | CAGAA | CAGAA |
| | Ex3 SA1 | TTCCA | UUCCA |
| | (Pos 9) | GTTCT | GUUCU |
| | | AAGGG | AAGGG |
| | | GAGCG | GAGCG |
| | Ex3 SD1 | TCACC | UCACC |
| | (pos 4) | TGAAA | UGAAA |
| | | TGACA | UGACA |
| | | ATGAT | AUGAU |
| | Ex6 SA1 | CATCC | CAUCC |
| | (pos 9) | AACCT | AACCU |
| | | GAAGA | GAAGA |
| | | CCAAA | CCAAA |
| | Ex8 SA1 | GATCT | GAUCU |
| | (Pos 6) | CTGAG | CUGAG |
| | | TGGAA | UGGAA |
| | | AGAAC | AGAAC |
| TNFRSF8 | Exon 1 ATG | AGGAC | AGGAC |
| (CD30) | | GCGCA | GCGCA |
| | | TCCCC | UCCCC |
| | | GGGGC | GGGGC |
| | Exon 1 STOP 2 | TCCCA | UCCCA |
| | | CAGGT | CAGGU |
| | | AAGCG | AAGCG |
| | | GGTGA | GGUGA |
| | Exon 1 STOP 1 | GGCGC | GGCGC |
| | | TACGA | UACGA |
| | | GCCTT | GCCUU |
| | | CCCAC | CCCAC |
| | Exon 2 SD | CCCAC | CCCAC |
| | | TCACC | UCACC |
| | | CATGG | CAUGG |
| | | GGCAG | GGCAG |
| | Exon 3 STOP | CGACA | CGACA |
| | | CAGCA | CAGCA |
| | | GTGCC | GUGCC |
| | | CACAG | CACAG |
| | Exon 4 SA 3 | GTCGT | GUCGU |
| | | CTAAG | CUAAG |
| | | GGACA | GGACA |
| | | CAGAC | CAGAC |
| | Exon 4 SA 2 | TCGTC | UCGUC |
| | | TAAGG | UAAGG |
| | | GACAC | GACAC |
| | | AGACA | AGACA |
| | Exon 4 SA 1 | CGTCT | CGUCU |
| | | AAGGG | AAGGG |
| | | ACACA | ACACA |
| | | GACAG | GACAG |
| | Exon 6 STOP | CCCCC | CCCCC |
| | | AGGCC | AGGCC |
| | | AAGCC | AAGCC |
| | | CACCC | CACCC |
| | Exon 6 SD | AAATT | AAAUU |
| | | ACCTG | ACCUG |
| | | GATCT | GAUCU |
| | | GAACT | GAACU |
| | Exon 8 SA | TCATC | UCAUC |
| | | TAAGG | UAAGG |
| | | GACAC | GACAC |
| | | AGATG | AGAUG |
| | Exon 10 SA | GTGCT | GUGCU |
| | | GCGGG | GCGGG |
| | | GAGAA | GAGAA |
| | | GCCCA | GCCCA |
| | Exon 10 SD 2 | ACCAT | ACCAU |
| | | TACCT | UACCU |
| | | GCATC | GCAUC |
| | | CAGAA | CAGAA |
| | Exon 10 SD 1 | CCATT | CCAUU |
| | | ACCTG | ACCUG |
| | | CATCC | CAUCC |
| | | AGAAC | AGAAC |
| | Exon 10 STOP | CCCCA | CCCCA |
| | | CTCAG | CUCAG |
| | | AGCTT | AGCUU |
| | | GCTGG | GCUGG |
| | Exon 11 STOP 1 | ACCCA | ACCCA |
| | | GAAGA | GAAGA |
| | | GCACT | GCACU |
| | | GGCCC | GGCCC |
| | Exon 11 STOP 2 | AGGAT | AGGAU |
| | | CACCC | CACCC |
| | | AGAAG | AGAAG |
| | | AGCAC | AGCAC |
| | Exon 12 SA 2 | TGGAG | UGGAG |
| | | CTCTG | CUCUG |
| | | AAACG | AAACG |
| | | ACACC | ACACC |
| | Exon 12 SA 1 | AGCTC | AGCUC |
| | | TGAAA | UGAAA |
| | | CGACA | CGACA |
| | | CCAGG | CCAGG |
| | Exon 12 SD 2 | CTCAC | CUCAC |
| | | CCACA | CCACA |
| | | AGCTC | AGCUC |
| | | TAGCT | UAGCU |
| | Exon 12 SD 1 | TCACC | UCACC |
| | | CACAA | CACAA |
| | | GCTCT | GCUCU |
| | | AGCTT | AGCUU |
| | Exon 13 SD 2 | ACTTA | ACUUA |
| | | CCGTT | CCGUU |
| | | GAGCT | GAGCU |
| | | CCTCC | CCUCC |
| | Exon 13 SD 1 | CTTAC | CUUAC |
| | | CGTTG | CGUUG |
| | | AGCTC | AGCUC |
| | | CTCCT | CUCCU |
| | Exon 14 SA 1 | AGCTG | AGCUG |
| | | CTGTG | CUGUG |
| | | GGACG | GGACG |
| | | GGAAT | GGAAU |
| | Exon 14 SA 2 | CAGCT | CAGCU |
| | | GCTGT | GCUGU |
| | | GGGAC | GGGAC |
| | | GGGAA | GGGAA |
| | iso exon 11 SA | CTCCT | CUCCU |
| | | CAGCT | CAGCU |
| | | GCTGT | GCUGU |
| | | GGGAC | GGGAC |
| | Exon 14 STOP | GCCGC | GCCGC |
| | | TGCAG | UGCAG |
| | | GATGC | GAUGC |
| | | CAGCC | CAGCC |
| | Exon 14 SD | TGACT | UGACU |
| | | CACCA | CACCA |
| | | ATCTT | AUCUU |
| | | GTTAT | GUUAU |
| | Exon 15 SA 1 | TCTCT | UCUCU |
| | | GCAAG | GCAAG |
| | | GCAAA | GCAAA |
| | | AGGAT | AGGAU |
| | Exon 15 SA 2 | TTCTC | UUCUC |
| | | TGCAA | UGCAA |
| | | GGCAA | GGCAA |
| | | AAGGA | AAGGA |
| TNFRSF10B | Ex1 SD1 | ACTCA | ACUCA |
| | (Pos 6) | CCAAC | CCAAC |
| | | AGCAG | AGCAG |
| | | GACCG | GACCG |
| | Ex2 SA1 | GAGAC | GAGAC |
| | (Pos 6) | CTGTG | CUGUG |
| | | GGGAC | GGGAC |
| | | AAAGC | AAAGC |
| | Ex 3 SD1 | CTCAC | CUCAC |
| | (Pos 5) | CCTGT | CCUGU |
| | | GCGGC | GCGGC |
| | | ACTTC | ACUUC |
| | Ex4 SA1 | ACACC | ACACC |
| | (Pos 5) | TGGGT | UGGGU |
| | | ACACA | ACACA |
| | | CACAG | CACAG |
| | Ex6 SA1 | TGAGC | UGAGC |
| | (Pos 7) | TCTGG | UCUGG |
| | | AAAAA | AAAAA |
| | | GACAT | GACAU |
| | Ex8 SA1 | CCGGT | CCGGU |
| | (pos 8) | TCCTG | UCCUG |
| | | TAACA | UAACA |
| | | CATAG | CAUAG |
| | Ex8 SA2 | CGGTT | CGGUU |
| | (Pos 7) | CCTGT | CCUGU |
| | | AACAC | AACAC |
| | | ATAGT | AUAGU |
| TOX | Exon 1 SD 3 | GTTCA | GUUCA |
| | | CCTTG | CCUUG |
| | | TTGCA | UUGCA |
| | | ATAGT | AUAGU |
| | Exon 1 SD 2 | TTCAC | UUCAC |
| | | CTTGT | CUUGU |
| | | TGCAA | UGCAA |
| | | TAGTA | UAGUA |
| | Exon 1 SD 1 | TCACC | UCACC |
| | | TTGTT | UUGUU |
| | | GCAAT | GCAAU |
| | | AGTAG | AGUAG |
| | Exon 4 STOP | TCACA | UCACA |
| | | GCTAA | GCUAA |
| | | GTGCT | GUGCU |
| | | CAACT | CAACU |
| | Exon 5 STOP 1 | TGATA | UGAUA |
| | | CTCAG | CUCAG |
| | | GCCGC | GCCGC |
| | | CATCA | CAUCA |
| | Exon 5 STOP 2 | GATAC | GAUAC |
| | | TCAGG | UCAGG |
| | | CCGCC | CCGCC |
| | | ATCAA | AUCAA |
| | Exon 5 STOP 3 | GAGAA | GAGAA |
| | | GAGCA | GAGCA |
| | | AAAAC | AAAAC |
| | | AGGTA | AGGUA |
| | Exon 5 STOP 4 | AGAAG | AGAAG |
| | | AGCAA | AGCAA |
| | | AAACA | AAACA |
| | | GGTAA | GGUAA |
| | Exon 5 SD | CGTTA | CGUUA |
| | | CCTTG | CCUUG |
| | | GATAC | GAUAC |
| | | AAGGC | AAGGC |
| | Exon 7 STOP 1 | TCACC | UCACC |
| | | ATGCA | AUGCA |
| | | GCAGC | GCAGC |
| | | CCCTT | CCCUU |
| | Exon 7 STOP 2 | TGGGA | UGGGA |
| | | ACCAG | ACCAG |
| | | CTCCC | CUCCC |
| | | CATGC | CAUGC |
| | Exon 7 STOP 3 | CATGC | CAUGC |
| | | AGCAA | AGCAA |
| | | GTAAG | GUAAG |
| | | TGCAA | UGCAA |
| | Exon 7 SD 2 | TGCAC | UGCAC |
| | | TTACT | UUACU |
| | | TGCTG | UGCUG |
| | | CATGG | CAUGG |
| | Exon 7 SD 1 | GCACT | GCACU |
| | | TACTT | UACUU |
| | | GCTGC | GCUGC |
| | | ATGGT | AUGGU |
| | Exon 8 STOP 1 | AGCTG | AGCUG |
| | | CACAA | CACAA |
| | | GTTGT | GUUGU |
| | | CACCC | CACCC |
| | Exon 8 STOP 2 | CTCCC | CUCCC |
| | | CCACA | CCACA |
| | | ACCGG | ACCGG |
| | | TGGAC | UGGAC |
| | Exon 8 STOP 6 | TTATT | UUAUU |
| | | CCAGT | CCAGU |
| | | CCACC | CCACC |
| | | GGTTG | GGUUG |
| | Exon 8 STOP 5 | TATTC | UAUUC |
| | | CAGTC | CAGUC |
| | | CACCG | CACCG |
| | | GTTGT | GUUGU |
| | Exon 8 STOP 4 | ATTCC | AUUCC |
| | | AGTCC | AGUCC |
| | | ACCGG | ACCGG |
| | | TTGTG | UUGUG |
| | Exon 8 STOP 3 | TTCCA | UUCCA |
| | | GTCCA | GUCCA |
| | | CCGGT | CCGGU |
| | | TGTGG | UGUGG |
| TOX2 | Exon 1 ATG 1 | GTCCA | GUCCA |
| | | TGGCG | UGGCG |
| | | GGCGC | GGCGC |
| | | GGCGG | GGCGG |
| | Exon 1 ATG 2 | GACGT | GACGU |
| | | CCATG | CCAUG |
| | | GCGGG | GCGGG |
| | | CGCGG | CGCGG |
| | Exon 2 STOP 1 | TATGC | UAUGC |
| | | AGCAG | AGCAG |
| | | ACTCG | ACUCG |
| | | CACAG | CACAG |
| | Exon 2 STOP 2 | TTTCC | UUUCC |
| | | GCAGA | GCAGA |
| | | AGGTA | AGGUA |
| | | AGCAG | AGCAG |
| | Exon 3 SA 2 | TCAAA | UCAAA |
| | | CTAGA | CUAGA |
| | | ATAGA | AUAGA |
| | | GAGAG | GAGAG |
| | Exon 3 SA 1 | CAAAC | CAAAC |
| | | TAGAA | UAGAA |
| | | TAGAG | UAGAG |
| | | AGAGA | AGAGA |
| | Exon 3 SD | CACCC | CACCC |
| | | ACCTG | ACCUG |
| | | GCTGG | GCUGG |
| | | TTGAC | UUGAC |
| | Exon 5 SD | TGGAT | UGGAU |
| | | CTAAG | CUAAG |
| | | AGAGG | AGAGG |
| | | AGGAC | AGGAC |
| | Exon 5 STOP 1 | GATCC | GAUCC |
| | | AGGAG | AGGAG |
| | | ATGGT | AUGGU |
| | | CCACT | CCACU |
| | Exon 5 STOP 2 | GTCCC | GUCCC |
| | | AGCTC | AGCUC |
| | | ATCTC | AUCUC |
| | | GCAGA | GCAGA |
| | Exon 5 STOP 3 | TCATC | UCAUC |
| | | TCGCA | UCGCA |
| | | GATGG | GAUGG |
| | | GCATC | GCAUC |
| | Exon 5 STOP 4 | CTCCA | CUCCA |
| | | CTCAG | CUCAG |
| | | GAAGA | GAAGA |
| | | GGAGT | GGAGU |
| | Exon 6 STOP 1 | TGAGC | UGAGC |
| | | CGCAG | CGCAG |
| | | AAGCC | AAGCC |
| | | TGTGT | UGUGU |
| | Exon 6 STOP 2 | AGACA | AGACA |
| | | CTCAG | CUCAG |
| | | GCCGC | GCCGC |
| | | CATCA | CAUCA |
| | Exon 6 STOP 3 | GACAC | GACAC |
| | | TCAGG | UCAGG |
| | | CCGCC | CCGCC |
| | | ATCAA | AUCAA |
| | Exon 8 STOP 1 | GACCT | GACCU |
| | | GCAGG | GCAGG |
| | | CCTTC | CCUUC |
| | | CGCAG | CGCAG |
| | Exon 8 STOP 2 | ACCTG | ACCUG |
| | | CAGGC | CAGGC |
| | | CTTCC | CUUCC |
| | | GCAGT | GCAGU |
| | Exon 8 STOP 3 | CCTGC | CCUGC |
| | | AGGCC | AGGCC |
| | | TTCCG | UUCCG |
| | | CAGTG | CAGUG |
| | Exon 8 SD | CTGCT | CUGCU |
| | | TACCT | UACCU |
| | | GTGGC | GUGGC |
| | | CCTGG | CCUGG |
| | Exon 9 SA 2 | GGAAG | GGAAG |
| | | TCCTA | UCCUA |
| | | CAGAG | CAGAG |
| | | TGGGA | UGGGA |
| | Exon 9 SA 1 | AGTCC | AGUCC |
| | | TACAG | UACAG |
| | | AGTGG | AGUGG |
| | | GAAGG | GAAGG |
| | Exon 9 STOP 2 | GTCCC | GUCCC |
| | | AGTCC | AGUCC |
| | | CCGCT | CCGCU |
| | | GCTGG | GCUGG |
| | Exon 9 STOP 1 | TCCCA | UCCCA |
| | | GTCCC | GUCCC |
| | | CGCTG | CGCUG |
| | | CTGGT | CUGGU |
| | Exon 9 STOP 3 | GCTGT | GCUGU |
| | | CCCAG | CCCAG |
| | | TCCCC | UCCCC |
| | | GCTGC | GCUGC |
| | Exon 10 SA 1 | CAGGC | CAGGC |
| | | TGTGA | UGUGA |
| | | GAGAG | GAGAG |
| | | AGGAG | AGGAG |
| | Exon 10 SA 2 | GCAGG | GCAGG |
| | | CTGTG | CUGUG |
| | | AGAGA | AGAGA |
| | | GAGGA | GAGGA |
| | Exon 10 SA 3 | AGCAG | AGCAG |
| | | GCTGT | GCUGU |
| | | GAGAG | GAGAG |
| | | AGAGG | AGAGG |
| UBASH3A | Ex1 SD1 | TGCCA | UGCCA |
| | (Pos 7) | TCTCT | UCUCU |
| | | TCCTG | UCCUG |
| | | CCCTT | CCCUU |
| | Ex1 SD1 | GTACT | GUACU |
| | (Pos8) | CACGC | CACGC |
| | | GGTGT | GGUGU |
| | | GCACC | GCACC |
| | Ex5 SA1 | GGAAG | GGAAG |
| | (Pos 9) | TGCCT | UGCCU |
| | | GGGTG | GGGUG |
| | | AGGAC | AGGAC |
| | Ex7 SA1 | AGGGT | AGGGU |
| | (Pos 6) | CTAGA | CUAGA |
| | | AAAGA | AAAGA |
| | | GGCAA | GGCAA |
| | Ex7 SA2 | GGGTC | GGGUC |
| | (Pos 5) | TAGAA | UAGAA |
| | | AAGAG | AAGAG |
| | | GCAAA | GCAAA |
| | Ex7 SA3 | GGTCT | GGUCU |
| | (Pos 4) | AGAAA | AGAAA |
| | | AGAGG | AGAGG |
| | | CAAAG | CAAAG |
| | Ex9 SA1 | GGTAG | GGUAG |
| | (Pos 7) | CCTGG | CCUGG |
| | | GGGGT | GGGGU |
| | | GGGGC | GGGGC |
| | Ex11 SA1 | CCCCT | CCCCU |
| | (Pos 4) | GGAAA | GGAAA |
| | | ATAGT | AUAGU |
| | | GAAAA | GAAAA |
| | Ex11 SD1 | CTGAC | CUGAC |
| | (Pos 5) | CTTCC | CUUCC |
| | | AGGAT | AGGAU |
| | | GAGTT | GAGUU |
| | Ex11 SD2 Pos | GAGGT | GAGGU |
| | (7) | TCTCA | UCUCA |
| | | CTGAC | CUGAC |
| | | CTTCC | CUUCC |
| | Ex13 SA1 | GCGGG | GCGGG |
| | (Pos 7) | CCTGG | CCUGG |
| | | AAGGA | AAGGA |
| | | TGAGA | UGAGA |
| | Ex14 SD1 | GCGTA | GCGUA |
| | (Pos 6) | CCTTT | CCUUU |
| | | CTCAC | CUCAC |
| | | GAGTT | GAGUU |
| | Ex14 SD2 | CGCGT | CGCGU |
| | (Pos 7) | ACCTT | ACCUU |
| | | TCTCA | UCUCA |
| | | CGAGT | CGAGU |
| VHL | Ex1 SD1 | GGCCC | GGCCC |
| | (Pos 9) | GTACC | GUACC |
| | | TCGGT | UCGGU |
| | | AGCTG | AGCUG |
| | Ex1 STOP1 | GTCCC | GUCCC |
| | (Pos 4) | AGTTC | AGUUC |
| | | TCCGC | UCCGC |
| | | CCTCC | CCUCC |
| | Ex2 STOP1 | GGAAC | GGAAC |
| | (Pos 9) | AAGCC | AAGCC |
| | | AGGGT | AGGGU |
| | | CATGT | CAUGU |
| | Ex2 STOP2 | CAACC | CAACC |
| | (Pos 9) | CCTCC | CCUCC |
| | | ATCTC | AUCUC |
| | | CCAGC | CCAGC |
| | Ex3 SA1 | TGACC | UGACC |
| | (Pos 5) | TATCG | UAUCG |
| | | GGACA | GGACA |
| | | AGCAA | AGCAA |
| | Ex3 SD1 | AGTAC | AGUAC |
| | (Pos 5) | CTGGC | CUGGC |
| | | AGTGT | AGUGU |
| | | GATAT | GAUAU |
| | Ex4 STOP1 | ATGTG | AUGUG |
| | (Pos 6) | CAGAA | CAGAA |
| | | AGACC | AGACC |
| | | TGGAG | UGGAG |
| XBP1 | Ex1 STOP1 | GGGCA | GGGCA |
| | (Pos 4) | GCCCG | GCCCG |
| | | CCTCC | CCUCC |
| | | GCCGC | GCCGC |
| | Ex1 STOP2 | CGGCC | CGGCC |
| | (Pos 5) | AGGCC | AGGCC |
| | | CTGCC | CUGCC |
| | | GCTCA | GCUCA |

### Methods of Using Fusion Proteins Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins provided herein, and with at least one guide RNA, wherein the guide RNA is about 15-100 nucleotides long and comprises a sequence of at least 10 contiguous nucleotides that is complementary to a target sequence. In some embodiments, the 3′ end of the target sequence is immediately adjacent to a canonical PAM sequence (NGG). In some embodiments, the 3′ end of the target sequence is not immediately adjacent to a canonical PAM sequence (NGG). In some embodiments, the 3′ end of the target sequence is immediately adjacent to an AGC, GAG, TTT, GTG, or CAA sequence. In some embodiments, the 3′ end of the target sequence is immediately adjacent to an NGA, NGCG, NGN, NNGRRT, NNNRRT, NGCG, NGCN, NGTN, NGTN, NGTN, or 5′ (TTTV) sequence.

In some embodiments, a fusion protein of the invention is used for mutagenizing a target of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced.

It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins comprising a Cas9 domain and a cytidine or adenosine deaminase, as disclosed herein, to a target site, e.g., a site comprising a mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein.

### Base Editor Efficiency

Some aspects of the disclosure are based on the recognition that any of the base editors provided herein can modify a specific nucleotide base without generating a sizable proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate) a specific nucleotide within a nucleic acid, without generating a large number of insertions or deletions (i.e., indels) in the nucleic acid. In some embodiments, it is desirable to generate base editors that efficiently modify (e.g. mutate or methylate) a specific nucleotide within a nucleic acid, without generating a large number of insertions or deletions (i.e., indels) in the nucleic acid. In certain embodiments, any of the base editors provided herein can generate a greater proportion of intended modifications (e.g., methylations) versus indels. In certain embodiments, any of the base editors provided herein can generate a greater proportion of intended modifications (e.g., mutations) versus indels. In some embodiments, the base editors provided herein are capable of generating a ratio of intended mutations to indels that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, a number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation in a nucleic acid (e.g. a nucleic acid within a genome of a subject) without generating a considerable number of unintended mutations. In some embodiments, an intended mutation is a mutation that is generated by a specific base editor bound to a gRNA, specifically designed to generate the intended mutation. In some embodiments, the intended mutation is a mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended mutations:unintended mutations) that is greater than 1:1. In some embodiments, any of the base editors provided herein are capable of generating a ratio of intended mutations to unintended mutations that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described in the “Base Editor Efficiency” section, herein, may be applied to any of the fusion proteins, or methods of using the fusion proteins provided herein.

A base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence, and may affect the gene product. In essence therefore, the gene editing modification described herein may result in a modification of the gene, structurally and/or functionally, wherein the expression of the gene product may be modified, for example, the expression of the gene is knocked out; or conversely, enhanced, or, in some circumstances, the gene function or activity may be modified. Using the methods disclosed herein, a base editing efficiency may be determined as the knockdown efficiency of the gene in which the base editing is performed, wherein the base editing is intended to knockdown the expression of the gene. A knockdown level may be validated quantitatively by determining the expression level by any detection assay, such as assay for protein expression level, for example, by flow cytometry; assay for detecting RNA expression such as quantitative RT-PCR, northern blot analysis, or any other suitable assay such as pyrosequencing; and may be validated qualitatively by nucleotide sequencing reactions.

In some embodiments, the modification, e.g., single base edit results in at least 10% reduction of the gene targeted expression. In some embodiments, the base editing efficiency may result in at least 10% reduction of the gene targeted expression. In some embodiments, the base editing efficiency may result in at least 20% reduction of the gene targeted expression. In some embodiments, the base editing efficiency may result in at least 30% reduction of the gene targeted expression. In some embodiments, the base editing efficiency may result in at least 40% reduction of the gene targeted expression. In some embodiments, the base editing efficiency may result in at least 50% reduction of the gene targeted expression. In some embodiments, the base editing efficiency may result in at least 60% reduction of the targeted gene expression. In some embodiments, the base editing efficiency may result in at least 70% reduction of the targeted gene expression. In some embodiments, the base editing efficiency may result in at least 80% reduction of the targeted gene expression. In some embodiments, the base editing efficiency may result in at least 90% reduction of the targeted gene expression. In some embodiments, the base editing efficiency may result in at least 91% reduction of the targeted gene expression. In some embodiments, the base editing efficiency may result in at least 92% reduction of the targeted gene expression. In some embodiments, the base editing efficiency may result in at least 93% reduction of the targeted gene expression. In some embodiments, the base editing efficiency may result in at least 94% reduction of the targeted gene expression. In some embodiments, the base editing efficiency may result in at least 95% reduction of the targeted gene expression. In some embodiments, the base editing efficiency may result in at least 96% reduction of the targeted gene expression. In some embodiments, the base editing efficiency may result in at least 97% reduction of the targeted gene expression. In some embodiments, the base editing efficiency may result in at least 98% reduction of the targeted gene expression. In some embodiments, the base editing efficiency may result in at least 99% reduction of the targeted gene expression. In some embodiments, the base editing efficiency may result in knockout (100% knockdown of the gene expression) of the gene that is targeted.

In some embodiments, targeted modifications, e.g., single base editing, are used simultaneously to target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 different endogenous sequences for base editing with different guide RNAs. In some embodiments, targeted modifications, e.g. single base editing, are used to sequentially target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, or more different endogenous gene sequences for base editing with different guide RNAs.

In some embodiments, a single gene delivery event (e.g., by transduction, transfection, electroporation or any other method) can be used to target base editing of 5 sequences within a cell's genome. In some embodiments, a single gene delivery event can be used to target base editing of 6 sequences within a cell's genome. In some embodiments, a single gene delivery event can be used to target base editing of 7 sequences within a cell's genome. In some embodiments, a single electroporation event can be used to target base editing of 8 sequences within a cell's genome. In some embodiments, a single gene delivery event can be used to target base editing of 9 sequences within a cell's genome. In some embodiments, a single gene delivery event can be used to target base editing of 10 sequences within a cell's genome. In some embodiments, a single gene delivery event can be used to target base editing of 20 sequences within a cell's genome. In some embodiments, a single gene delivery event can be used to target base editing of 30 sequences within a cell's genome. In some embodiments, a single gene delivery event can be used to target base editing of 40 sequences within a cell's genome. In some embodiments, a single gene delivery event can be used to target base editing of 50 sequences within a cell's genome.

In some embodiments, the method described herein, for example, the base editing methods has minimum to no off-target effects.

In some embodiments, the base editing method described herein results in at least 50% of a cell population that have been successfully edited (i.e., cells that have been successfully engineered). In some embodiments, the base editing method described herein results in at least 55% of a cell population that have been successfully edited. In some embodiments, the base editing method described herein results in at least 60% of a cell population that have been successfully edited. In some embodiments, the base editing method described herein results in at least 65% of a cell population that have been successfully edited. In some embodiments, the base editing method described herein results in at least 70% of a cell population that have been successfully edited. In some embodiments, the base editing method described herein results in at least 75% of a cell population that have been successfully edited. In some embodiments, the base editing method described herein results in at least 80% of a cell population that have been successfully edited. In some embodiments, the base editing method described herein results in at least 85% of a cell population that have been successfully edited. In some embodiments, the base editing method described herein results in at least 90% of a cell population that have been successfully edited. In some embodiments, the base editing method described herein results in at least 95% of a cell population that have been successfully edited. In some embodiments, the base editing method described herein results in about 91%, 92%, 93%, 94%, 95%, 96%, 97%, 98%, 99% or 100% of a cell population that have been successfully edited.

In some embodiments, the live cell recovery following a base editing intervention is greater than at least 60%, 70%, 80%, 90% of the starting cell population at the time of the base editing event. In some embodiments, the live cell recovery as described above is about 70%. In some embodiments, the live cell recovery as described above is about 75%. In some embodiments, the live cell recovery as described above is about 80%. In some embodiments, the live cell recovery as described above is about 85%. In some embodiments, the live cell recovery as described above is about 90%, or about 91%, 92%, 93%, 94% 95%, 96%, 97%, 98%, or 99%, or 100% of the cells in the population at the time of the base editing event.

In some embodiments the engineered cell population can be further expanded in vitro by about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

### Methods for Editing Nucleic Acids

Some aspects of the disclosure provide methods for editing a nucleic acid. In some embodiments, the method is a method for editing a nucleobase of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to a cytidine or adenosine deaminase) and a guide nucleic acid (e.g., gRNA), wherein the target region comprises a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase. In some embodiments, the method results in less than 20% indel formation in the nucleic acid. It should be appreciated that in some embodiments, step b is omitted. In some embodiments, the method results in less than 19%, 18%, 16%, 14%, 12%, 10%, 8%, 6%, 4%, 2%, 1%, 0.5% 0.2%, or less than 0.1% indel formation. In some embodiments, the method further comprises replacing the second nucleobase with a fifth nucleobase that is complementary to the fourth nucleobase, thereby generating an intended edited base pair (e.g., C-G to T-A). In some embodiments, at least 5% of the intended base pairs are edited. In some embodiments, at least 10%, 15%, 20%, 25%, 30%, 35%, 40%, 45%, or 50% of the intended base pairs are edited.

In some embodiments, the ratio of intended products to unintended products in the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended mutation to indel formation is greater than 1:1, 10:1, 50:1, 100:1, 500:1, or 1000:1, or more. In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the base editor protects or binds the non-edited strand. In some embodiments, the base editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the nucleobase editor comprises a linker. In some embodiments, the linker is 1-25 amino acids in length. In some embodiments, the linker is 5-20 amino acids in length. In some embodiments, linker is 10, 11, 12, 13, 14, 15, 16, 17, 18, 19, or 20 amino acids in length. In one embodiment, the linker is 32 amino acids in length. In another embodiment, a “long linker” is at least about 60 amino acids in length. In other embodiments, the linker is between about 3-100 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the base editors provided herein. In some embodiments, a target window is a methylation window.

In some embodiments, the disclosure provides methods for editing a nucleotide. In some embodiments, the disclosure provides a method for editing a nucleobase pair of a double-stranded DNA sequence. In some embodiments, the method comprises a) contacting a target region of the double-stranded DNA sequence with a complex comprising a base editor and a guide nucleic acid (e.g., gRNA), where the target region comprises a target nucleobase pair, b) inducing strand separation of said target region, c) converting a first nucleobase of said target nucleobase pair in a single strand of the target region to a second nucleobase, d) cutting no more than one strand of said target region, wherein a third nucleobase complementary to the first nucleobase base is replaced by a fourth nucleobase complementary to the second nucleobase, and the second nucleobase is replaced with a fifth nucleobase that is complementary to the fourth nucleobase, thereby generating an intended edited base pair, wherein the efficiency of generating the intended edited base pair is at least 5%. It should be appreciated that in some embodiments, step b is omitted. In some embodiments, at least 5% of the intended base pairs are edited. In some embodiments, at least 10%, 15%, 20%, 25%, 30%, 35%, 40%, 45%, or 50% of the intended base pairs are edited. In some embodiments base editing by a method described herein may have a base conversion efficiency of at least 10% at any particular gene site. In some embodiments, base editing by a method described herein may have a base conversion efficiency of at least 20%, at least 25%, at least 30%, at least 35%, at least 40%, at least 45%, at least 50% at least 55% or at least 60%, at least 65%, at least 70%, at least 75%, at least 80%, at least 85%, at least 90%, or at least 95%, 96%, 97%, 98% or at least 99% at any particular gene site. In some embodiments base editing by a method described herein may have a base conversion efficiency of at least 70% at any particular gene site. In some embodiments base editing by a method described herein may have a base conversion efficiency of at least 80% at any particular gene site. In some embodiments base editing by a method described herein may have a base conversion efficiency of at least 90% at any particular gene site.

In some embodiments, the method causes less than 19%, 18%, 16%, 14%, 12%, 10%, 8%, 6%, 4%, 2%, 0.5%, 0.2%, or less than 0.1% indel formation. In some embodiments, the ratio of intended product to unintended products at the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended mutation to indel formation is greater than 1:1, 10:1, 50:1, 100:1, 500:1, or 1000:1, or more. In some embodiments, the cut single strand is hybridized to the guide nucleic acid. In some embodiments, the cut single strand is opposite to the strand comprising the first nucleobase. In some embodiments, the nucleobase editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the nucleobase editor comprises a linker. In some embodiments, the linker is 1-25 amino acids in length. In some embodiments, the linker is 5-20 amino acids in length. In some embodiments, the linker is 10, 11, 12, 13, 14, 15, 16, 17, 18, 19, or 20 amino acids in length. e.g., In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair occurs within the target window. In some embodiments, the target window comprises the intended edited base pair. In some embodiments, the nucleobase editor is any one of the base editors provided herein.

### Nucleic Acid-Based Delivery of Cytidine or Adenosine Deaminase Nucleobase Editor

Nucleic acids encoding a cytidine or adenosine deaminase nucleobase editor according to the present disclosure can be administered to subjects or delivered into cells by art-known methods or as described herein. For example, cytidine or adenosine deaminase nucleobase editors can be delivered by, e.g., vectors (e.g., viral or non-viral vectors), non-vector based methods (e.g., using naked DNA or DNA complexes), or a combination thereof.

Nucleic acids encoding cytidine or adenosine deaminase nucleobase editors can be delivered directly to cells as naked DNA or RNA, for instance by means of transfection or electroporation, or can be conjugated to molecules (e.g., N-acetylgalactosamine) promoting uptake by the target cells. Nucleic acid vectors, such as the vectors can also be used. In particular embodiments, a polynucleotide, e.g. a mRNA encoding a base editor or a functional component thereof may be co-electroporated with a combination of multiple guide RNAs as described herein.

Nucleic acid vectors can comprise one or more sequences encoding a domain of a fusion protein described herein. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40), and one or more deaminases.

The nucleic acid vector can also include any suitable number of regulatory/control elements, e.g., promoters, enhancers, introns, polyadenylation signals, Kozak consensus sequences, or internal ribosome entry sites (IRES). These elements are well known in the art.

Nucleic acid vectors according to this disclosure include recombinant viral vectors. Exemplary viral vectors are set forth herein above. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 9 (below).

TABLE 9
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1 -Cholesteryloxycarbonyl-3,7-diazanonane-1,9-diamine | CDAN | Cationic |
| 2(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 10 lists exemplary polymers for use in gene transfer and/or nanoparticle formulations.

TABLE 10
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

The following Table 11 summarizes delivery methods for a polynucleotide encoding a fusion protein described herein.

TABLE 11

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In particular embodiments, a fusion protein of the invention is encoded by a polynucleotide present in a viral vector (e.g., adeno-associated virus (AAV), AAV3, AAV3b, AAV4, AAV5, AAV6, AAV7, AAV8, AAV9, AAVrh8, AAV10, and variants thereof), or a suitable capsid protein of any viral vector. Thus, in some aspects, the disclosure relates to the viral delivery of a fusion protein. Examples of viral vectors include retroviral vectors (e.g. Maloney murine leukemia virus, MML-V), adenoviral vectors (e.g. AD100), lentiviral vectors (HIV and FIV-based vectors), herpesvirus vectors (e.g. HSV-2).

In one embodiment, inteins are utilized to join fragments or portions of a cytidine or adenosine deaminase base editor protein that is grafted onto an AAV capsid protein. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

A fragment of a fusion protein of the invention can vary in length. In some embodiments, a protein fragment ranges from 2 amino acids to about 1000 amino acids in length. In some embodiments, a protein fragment ranges from about 5 amino acids to about 500 amino acids in length. In some embodiments, a protein fragment ranges from about 20 amino acids to about 200 amino acids in length. In some embodiments, a protein fragment ranges from about 10 amino acids to about 100 amino acids in length. Suitable protein fragments of other lengths will be apparent to a person of skill in the art.

In some embodiments, a portion or fragment of a nuclease (e.g., a fragment of a deaminase, such as cytidine or adenosine deaminase, or a fragment of Cas9) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In some aspects, the methods described herein for editing specific genes in an immune cell can be used to genetically modify a CAR-T cell. Such CAR-T cells, and methods to produce such CAR-T cells are described in International Application Nos. PCT/US2016/060736, PCT/US2016/060734, PCT/US2016/034873, PCT/US2015/040660, PCT/EP2016/055332, PCT/IB2015/058650, PCT/EP2015/067441, PCT/EP2014/078876, PCT/EP2014/059662, PCT/IB2014/061409, PCT/US2016/019192, PCT/US2015/059106, PCT/US2016/052260, PCT/US2015/020606, PCT/US2015/055764, PCT/CN2014/094393, PCT/US2017/059989, PCT/US2017/027606, and PCT/US2015/064269, the contents of each is hereby incorporated in its entirety.

### Pharmaceutical Compositions

In some aspects, the present invention provides a pharmaceutical composition comprising a genetically modified immune cell of the present invention. More specifically, provided herein are pharmaceutical compositions comprising a genetically modified immune cell, or a population of such immune cells, expressing a chimeric antigen receptor, wherein said modified immune cell, or a population thereof, has at least one edited gene edited to enhance the function of the modified immune cell or to reduce immunosuppression or inhibition of the modified immune cell, wherein expression of the edited gene is either knocked out or knocked down. In some embodiments the at least one edited gene is TRAC, B2M, PDCD1, CBLB, TGFBR2, ZAP70, NFATc1, TET2, or combination thereof.

The pharmaceutical compositions of the present invention can be prepared in accordance with known techniques. See, e.g., Remington, The Science And Practice of Pharmacy (21st ed. 2005). In general, the immune cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In addition to the modified immune cell, or population thereof, and the carrier, the pharmaceutical compositions of the present invention can include at least one additional therapeutic agent useful in the treatment of disease. For example, some embodiments of the pharmaceutical composition described herein further comprise a chemotherapeutic agent. In some embodiments, the pharmaceutical composition further comprises a cytokine peptide or a nucleic acid sequence encoding a cytokine peptide. In some embodiments, the pharmaceutical compositions comprising the modified immune cell or population thereof can be administered separately from an additional therapeutic agent.

The pharmaceutical compositions of the present invention can be used to treat any disease or condition that is responsive to autologous or allogeneic immune cell immunotherapy. For example, the pharmaceutical compositions, in some embodiments are useful in the treatment of neoplasia. In some embodiments, the neoplasia is a hematological cancer. In some embodiments, the hematological cancer is a B cell cancer, and in some embodiments, the B cell cancer is multiple myeloma. In some embodiments, the B cell cancer is relapsed of relapsed/refractory multiple myeloma.

One consideration concerning the therapeutic use of genetically modified immune cells of the invention is the quantity of cells necessary to achieve an optimal or satisfactory effect. The quantity of cells to be administered may vary for the subject being treated. In one embodiment, between 10⁴ to 10¹⁰, between 10⁵ to 10⁹, or between 10⁶ and 10⁸ genetically modified immunoresponsive cells of the invention are administered to a human subject. In some embodiments, at least about 1×10⁸, 2×10⁸, 3×10⁸, 4×10⁸, and 5×10⁸ genetically modified immune cells of the invention are administered to a human subject. Determining the precise effective dose may be based on factors for each individual subject, including their size, age, sex, weight, and condition. Dosages can be readily ascertained by those skilled in the art from this disclosure and the knowledge in the art.

The skilled artisan can readily determine the number of cells and amount of optional additives, vehicles, and/or carriers in compositions and to be administered in methods of the invention. Typically, additives (in addition to the active immune cell(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD50 in a suitable animal model (e.g., a rodent such as a mouse); and, the dosage of the composition(s), concentration of components therein, and the timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

In one embodiment, the method and compositions described herein may be used in generating engineered T cells that express a CAR and may have one or more base edited modifications, such that the engineered T cell can mount a specific immune response against the target. The CAR may be specifically directed towards an antigen target, the antigen may be presented by a cell in a host. In some embodiments, the immune response encompasses cytotoxicity. In some embodiments, the engineered T cell has enhanced cytotoxic response against its target. In some embodiments, the engineered T cell induces an enhanced cytotoxic response against its target as compared to a non-engineered T cell. In some embodiments, the engineered T cell exhibits an enhanced cytotoxic response by at least 1.1-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold or more compared to a non-engineered cell. In some embodiments, the engineered T cell can kill at least 10%, at least 20%, at least 30%, at least 40%, at least 50%, at least 60%, at least 70%, at least 80%, at least 90%, at least 100%, at least 200%, at least 500% or at least 1000% more target cells than a non-engineered cell. In some embodiments, the T cell can induce higher memory response. In some embodiments, the T cell can induce lower levels of inflammatory cytokines than a non-engineered cell, that is, the engineered cell does not cause a cytokine storm response. In some embodiments, the engineered T cell is administered to an allogenic host, wherein the engineered T cell has no rejection by the host. In some embodiments, the allogenic T cell induces negligible or minimum rejection by the host.

### Methods of Treatment

Some aspects of the present invention provide methods of treating a subject in need, the method comprising administering to a subject in need an effective therapeutic amount of a pharmaceutical composition as described herein. More specifically, the methods of treatment comprise administering to a subject in need thereof a pharmaceutical composition comprising a population of modified immune cells expressing a chimeric receptor and having at least one edited gene, wherein the at least one edited gene enhances the function or reduces the immunosuppression or inhibition of the modified immune cell, and wherein expression of the at least one edited gene is either knocked out or knocked down. In some embodiments, the method of treatment is an autologous immune cell therapy. In other embodiments, the method of treatment is an allogeneic immune cell therapy.

In certain embodiments, the specificity of an immune cell is redirected to a marker expressed on the surface of a diseased or altered cell in a subject by genetically modifying the immune cell to express a chimeric antigen receptor contemplated herein. In some embodiments, the method of treatment comprises administering to a subject an immune cell as described herein, wherein the immune cell has been genetically modified to redirect its specificity to a marker expressed on a neoplastic cell. In some embodiments, the neoplasia is a B cell cancer; for example, a B cell cancer such as a lymphoma, leukemia, or a myeloma, for example, multiple myeloma. Thus, some embodiments of the present disclosure provide a method of treating a neoplasia in a subject. In some embodiments, the neoplasia being treated is a B cell cancer. In some embodiments, the B cell cancer is a lymphoma, leukemia, or multiple myeloma.

Some embodiments of the methods of treating a neoplasia in a subject comprise administering to the subject an immune cell as described herein and one or more additional therapeutic agents. For example, the immune cell of the present invention can be co-administered with a cytokine. In some embodiments, the cytokine is IL-2, IFN-á, IFN-ã, or a combination thereof. In some embodiments, the immune cell is co-administered with a chemotherapeutic agent. The chemotherapeutic can be cyclophosphamide, doxorubicin, vincristine, prednisone, or rituximab, or a combination thereof. Other chemotherapeutics include obinutuzumab, bendamustine, chlorambucil, cyclophosphamide, ibrutinib, methotrexate, cytarabine, dexamethasone, cisplatin, bortezomib, fludarabine, idelalisib, acalabrutinib, lenalidomide, venetoclax, cyclophosphamide, ifosfamide, etoposide, pentostatin, melphalan, carfilzomib, ixazomib, panobinostat, daratumumab, elotuzumab, thalidomide, lenalidomide, or pomalidomide, or a combination thereof. “Co-administered” refers to administering two or more therapeutic agents or pharmaceutical compositions during a course of treatment. Such co-administration can be simultaneous administration or sequential administration. Sequential administration of a later-administered therapeutic agent or pharmaceutical composition can occur at any time during the course of treatment after administration of the first pharmaceutical composition or therapeutic agent.

In some embodiments of the present invention, an administered immune cell proliferates in vivo and can persist in the subject for an extended period of time. Immune cells of the present invention, in some embodiments can mature into memory immune cells and remain in circulation within the subject, thereby generating a population of cells able to actively respond to recurrence of a diseased or altered cell expressing the marker recognized by the chimeric antigen receptor.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

Kits, Vectors, Cells

The invention also provides kits comprising a nucleic acid construct comprising a nucleotide sequence encoding a cytidine or adenosine deaminase nucleobase editor at least two guide RNAs, each guide RNA having a nucleic acid sequence at least 85% complementary to a nucleic acid sequence of gene encoding TRAC, B2M, PD1, CBLB, and/or CTLA4. In some embodiments, the nucleotide sequence encoding the cytidine or adenosine deaminase comprises a heterologous promoter that drives expression of the cytidine or adenosine deaminase nucleobase editor.

Some aspects of this disclosure provide kits comprising a nucleic acid construct, comprising (a) a nucleotide sequence encoding (a) a Cas9 domain fused to a cytidine or adenosine deaminase as provided herein; and (b) a heterologous promoter that drives expression of the sequence of (a).

Some aspects of this disclosure provide kits for the treatment of a neoplasia comprising a modified immune cell or immune cell having reduced immunogenicity and enhanced anti-neoplasia activity, the immune or immune cell comprising a mutation in a TRAC, B2M, PD1, CBLB, and/or CTLA4 polypeptide, or a combination thereof. In some embodiments, the modified immune cell further comprises a chimeric antigen receptor having an affinity for a marker associated with the neoplasia. The neoplasia treatment kits comprise written instructions for using the modified immune cells in the treatment of the neoplasia.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Disruption of Splice Sites and Introduction of Stop Codons in Genes Expressed in Immune Cells

A nucleobase editor, BE4, was used to disrupt splice sites and insert stop codons into a subset of genes expressed in immune cells. A plasmid construct, pCMV_BE4max, encodes BE4, which comprises an APOBEC-1 cytidine deaminase domain having cytidine deaminase activity, a Cas9 domain comprising a D10A mutation and having nicknase activity, and two uracil DNA glycosylase inhibitor (UGI) domains. UGI is an 83-amino acid residue protein derived from Bacillus subtilis bacteriophage PBS1 that potently blocks to edit the splice sites of certain genes expressed in immune cells. BE4 further comprises N-terminal and C-terminal nuclear localization signals (NLSs).

>pCMV_BE4max
ATATGCCAAGTACGCCCCCTATTGACGTCAATGACGGTAAATGGCCCGCCTGGCATTAT
GCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTG
ACTCACGGGGATTTCCAAGTCTCCACCCCATTGACGTCAATGGGAGTTTGTTTTGGCAC
CAAAATCAACGGGACTTTCCAAAATGTCGTAACAACTCCGCCCCATTGACGCAAATGGG
CGGTAGGCGTGTACGGTGGGAGGTCTATATAAGCAGAGCTGGTTTAGTGAACCGTCAGA
TCCGCTAGAGATCCGCGGCCGCTAATACGACTCACTATAGGGAGAGCCGCCACCATGAA
ACGGACAGCCGACGGAAGCGAGTTCGAGTCACCAAAGAAGAAGCGGAAAGTCTCCTCAG
AGACTGGGCCTGTCGCCGTCGATCCAACCCTGCGCCGCCGGATTGAACCTCACGAGTTT
GAAGTGTTCTTTGACCCCCGGGAGCTGAGAAAGGAGACATGCCTGCTGTACGAGATCAA
CTGGGGAGGCAGGCACTCCATCTGGAGGCACACCTCTCAGAACACAAATAAGCACGTGG
AGGTGAACTTCATCGAGAAGTTTACCACAGAGCGGTACTTCTGCCCCAATACCAGATGT
AGCATCACATGGTTTCTGAGCTGGTCCCCTTGCGGAGAGTGTAGCAGGGCCATCACCGA
GTTCCTGTCCAGATATCCACACGTGACACTGTTTATCTACATCGCCAGGCTGTATCACC
ACGCAGACCCAAGGAATAGGCAGGGCCTGCGCGATCTGATCAGCTCCGGCGTGACCATC
CAGATCATGACAGAGCAGGAGTCCGGCTACTGCTGGCGGAACTTCGTGAATTATTCTCC
TAGCAACGAGGCCCACTGGCCTAGGTACCCACACCTGTGGGTGCGCCTGTACGTGCTGG
AGCTGTATTGCATCATCCTGGGCCTGCCCCCTTGTCTGAATATCCTGCGGAGAAAGCAG
CCCCAGCTGACCTTCTTTACAATCGCCCTGCAGTCTTGTCACTATCAGAGGCTGCCACC
CCACATCCTGTGGGCCACAGGCCTGAAGTCTGGAGGATCTAGCGGAGGATCCTCTGGCA
GCGAGACACCAGGAACAAGCGAGTCAGCAACACCAGAGAGCAGTGGCGGCAGCAGCGGC
GGCAGCGACAAGAAGTACAGCATCGGCCTGGCCATCGGCACCAACTCTGTGGGCTGGGC
CGTGATCACCGACGAGTACAAGGTGCCCAGCAAGAAATTCAAGGTGCTGGGCAACACCG
ACCGGCACAGCATCAAGAAGAACCTGATCGGAGCCCTGCTGTTCGACAGCGGCGAAACA
GCCGAGGCCACCCGGCTGAAGAGAACCGCCAGAAGAAGATACACCAGACGGAAGAACCG
GATCTGCTATCTGCAAGAGATCTTCAGCAACGAGATGGCCAAGGTGGACGACAGCTTCT
TCCACAGACTGGAAGAGTCCTTCCTGGTGGAAGAGGATAAGAAGCACGAGCGGCACCCC
ATCTTCGGCAACATCGTGGACGAGGTGGCCTACCACGAGAAGTACCCCACCATCTACCA
CCTGAGAAAGAAACTGGTGGACAGCACCGACAAGGCCGACCTGCGGCTGATCTATCTGG
CCCTGGCCCACATGATCAAGTTCCGGGGCCACTTCCTGATCGAGGGCGACCTGAACCCC
GACAACAGCGACGTGGACAAGCTGTTCATCCAGCTGGTGCAGACCTACAACCAGCTGTT
CGAGGAAAACCCCATCAACGCCAGCGGCGTGGACGCCAAGGCCATCCTGTCTGCCAGAC
TGAGCAAGAGCAGACGGCTGGAAAATCTGATCGCCCAGCTGCCCGGCGAGAAGAAGAAT
GGCCTGTTCGGAAACCTGATTGCCCTGAGCCTGGGCCTGACCCCCAACTTCAAGAGCAA
CTTCGACCTGGCCGAGGATGCCAAACTGCAGCTGAGCAAGGACACCTACGACGACGACC
TGGACAACCTGCTGGCCCAGATCGGCGACCAGTACGCCGACCTGTTTCTGGCCGCCAAG
AACCTGTCCGACGCCATCCTGCTGAGCGACATCCTGAGAGTGAACACCGAGATCACCAA
GGCCCCCCTGAGCGCCTCTATGATCAAGAGATACGACGAGCACCACCAGGACCTGACCC
TGCTGAAAGCTCTCGTGCGGCAGCAGCTGCCTGAGAAGTACAAAGAGATTTTCTTCGAC
CAGAGCAAGAACGGCTACGCCGGCTACATTGACGGCGGAGCCAGCCAGGAAGAGTTCTA
CAAGTTCATCAAGCCCATCCTGGAAAAGATGGACGGCACCGAGGAACTGCTCGTGAAGC
TGAACAGAGAGGACCTGCTGCGGAAGCAGCGGACCTTCGACAACGGCAGCATCCCCCAC
CAGATCCACCTGGGAGAGCTGCACGCCATTCTGCGGCGGCAGGAAGATTTTTACCCATT
CCTGAAGGACAACCGGGAAAAGATCGAGAAGATCCTGACCTTCCGCATCCCCTACTACG
TGGGCCCTCTGGCCAGGGGAAACAGCAGATTCGCCTGGATGACCAGAAAGAGCGAGGAA
ACCATCACCCCCTGGAACTTCGAGGAAGTGGTGGACAAGGGCGCTTCCGCCCAGAGCTT
CATCGAGCGGATGACCAACTTCGATAAGAACCTGCCCAACGAGAAGGTGCTGCCCAAGC
ACAGCCTGCTGTACGAGTACTTCACCGTGTATAACGAGCTGACCAAAGTGAAATACGTG
ACCGAGGGAATGAGAAAGCCCGCCTTCCTGAGCGGCGAGCAGAAAAAGGCCATCGTGGA
CCTGCTGTTCAAGACCAACCGGAAAGTGACCGTGAAGCAGCTGAAAGAGGACTACTTCA
AGAAAATCGAGTGCTTCGACTCCGTGGAAATCTCCGGCGTGGAAGATCGGTTCAACGCC
TCCCTGGGCACATACCACGATCTGCTGAAAATTATCAAGGACAAGGACTTCCTGGACAA
TGAGGAAAACGAGGACATTCTGGAAGATATCGTGCTGACCCTGACACTGTTTGAGGACA
GAGAGATGATCGAGGAACGGCTGAAAACCTATGCCCACCTGTTCGACGACAAAGTGATG
AAGCAGCTGAAGCGGCGGAGATACACCGGCTGGGGCAGGCTGAGCCGGAAGCTGATCAA
CGGCATCCGGGACAAGCAGTCCGGCAAGACAATCCTGGATTTCCTGAAGTCCGACGGCT
TCGCCAACAGAAACTTCATGCAGCTGATCCACGACGACAGCCTGACCTTTAAAGAGGAC
ATCCAGAAAGCCCAGGTGTCCGGCCAGGGCGATAGCCTGCACGAGCACATTGCCAATCT
GGCCGGCAGCCCCGCCATTAAGAAGGGCATCCTGCAGACAGTGAAGGTGGTGGACGAGC
TCGTGAAAGTGATGGGCCGGCACAAGCCCGAGAACATCGTGATCGAAATGGCCAGAGAG
AACCAGACCACCCAGAAGGGACAGAAGAACAGCCGCGAGAGAATGAAGCGGATCGAAGA
GGGCATCAAAGAGCTGGGCAGCCAGATCCTGAAAGAACACCCCGTGGAAAACACCCAGC
TGCAGAACGAGAAGCTGTACCTGTACTACCTGCAGAATGGGCGGGATATGTACGTGGAC
CAGGAACTGGACATCAACCGGCTGTCCGACTACGATGTGGACCATATCGTGCCTCAGAG
CTTTCTGAAGGACGACTCCATCGACAACAAGGTGCTGACCAGAAGCGACAAGAACCGGG
GCAAGAGCGACAACGTGCCCTCCGAAGAGGTCGTGAAGAAGATGAAGAACTACTGGCGG
CAGCTGCTGAACGCCAAGCTGATTACCCAGAGAAAGTTCGACAATCTGACCAAGGCCGA
GAGAGGCGGCCTGAGCGAACTGGATAAGGCCGGCTTCATCAAGAGACAGCTGGTGGAAA
CCCGGCAGATCACAAAGCACGTGGCACAGATCCTGGACTCCCGGATGAACACTAAGTAC
GACGAGAATGACAAGCTGATCCGGGAAGTGAAAGTGATCACCCTGAAGTCCAAGCTGGT
GTCCGATTTCCGGAAGGATTTCCAGTTTTACAAAGTGCGCGAGATCAACAACTACCACC
ACGCCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGATGAT
CGCCAAGAGCGAGCAGGAAATCGGCAAGGCTACCGCCAAGTACTTCTTCTACAGCAACA
TCATGAACTTTTTCAAGACCGAGATTACCCTGGCCAACGGCGAGATCCGGAAGCGGCCT
CTGATCGAGACAAACGGCGAAACCGGGGAGATCGTGTGGGATAAGGGCCGGGATTTTGC
CACCGTGCGGAAAGTGCTGAGCATGCCCCAAGTGAATATCGTGAAAAAGACCGAGGTGC
AGACAGGCGGCTTCAGCAAAGAGTCTATCCTGCCCAAGAGGAACAGCGATAAGCTGATC
GCCAGAAAGAAGGACTGGGACCCTAAGAAGTACGGCGGCTTCGACAGCCCCACCGTGGC
CTATTCTGTGCTGGTGGTGGCCAAAGTGGAAAAGGGCAAGTCCAAGAAACTGAAGAGTG
TGAAAGAGCTGCTGGGGATCACCATCATGGAAAGAAGCAGCTTCGAGAAGAATCCCATC
GACTTTCTGGAAGCCAAGGGCTACAAAGAAGTGAAAAAGGACCTGATCATCAAGCTGCC
TAAGTACTCCCTGTTCGAGCTGGAAAACGGCCGGAAGAGAATGCTGGCCTCTGCCGGCG
AACTGCAGAAGGGAAACGAACTGGCCCTGCCCTCCAAATATGTGAACTTCCTGTACCTG
GCCAGCCACTATGAGAAGCTGAAGGGCTCCCCCGAGGATAATGAGCAGAAACAGCTGTT
TGTGGAACAGCACAAGCACTACCTGGACGAGATCATCGAGCAGATCAGCGAGTTCTCCA
AGAGAGTGATCCTGGCCGACGCTAATCTGGACAAAGTGCTGTCCGCCTACAACAAGCAC
CGGGATAAGCCCATCAGAGAGCAGGCCGAGAATATCATCCACCTGTTTACCCTGACCAA
TCTGGGAGCCCCTGCCGCCTTCAAGTACTTTGACACCACCATCGACCGGAAGAGGTACA
CCAGCACCAAAGAGGTGCTGGACGCCACCCTGATCCACCAGAGCATCACCGGCCTGTAC
GAGACACGGATCGACCTGTCTCAGCTGGGAGGTGACAGCGGCGGGAGCGGCGGGAGCGG
GGGGAGCACTAATCTGAGCGACATCATTGAGAAGGAGACTGGGAAACAGCTGGTCATTC
AGGAGTCCATCCTGATGCTGCCTGAGGAGGTGGAGGAAGTGATCGGCAACAAGCCAGAG
TCTGACATCCTGGTGCACACCGCCTACGACGAGTCCACAGATGAGAATGTGATGCTGCT
GACCTCTGACGCCCCCGAGTATAAGCCTTGGGCCCTGGTCATCCAGGATTCTAACGGCG
AGAATAAGATCAAGATGCTGAGCGGAGGATCCGGAGGATCTGGAGGCAGCACCAACCTG
TCTGACATCATCGAGAAGGAGACAGGCAAGCAGCTGGTCATCCAGGAGAGCATCCTGAT
GCTGCCCGAAGAAGTCGAAGAAGTGATCGGAAACAAGCCTGAGAGCGATATCCTGGTCC
ATACCGCCTACGACGAGAGTACCGACGAAAATGTGATGCTGCTGACATCCGACGCCCCA
GAGTATAAGCCCTGGGCTCTGGTCATCCAGGATTCCAACGGAGAGAACAAAATCAAAAT
GCTGTCTGGCGGCTCAAAAAGAACCGCCGACGGCAGCGAATTCGAGCCCAAGAAGAAGA
GGAAAGTCTAACCGGTCATCATCACCATCACCATTGAGTTTAAACCCGCTGATCAGCCT
CGACTGTGCCTTCTAGTTGCCAGCCATCTGTTGTTTGCCCCTCCCCCGTGCCTTCCTTG
ACCCTGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGGACAGCAAGGGGG
AGGATTGGGAAGACAATAGCAGGCATGCTGGGGATGCGGTGGGCTCTATGGCTTCTGAG
GCGGAAAGAACCAGCTGGGGCTCGATACCGTCGACCTCTAGCTAGAGCTTGGCGTAATC
ATGGTCATAGCTGTTTCCTGTGTGAAATTGTTATCCGCTCACAATTCCACACAACATAC
GAGCCGGAAGCATAAAGTGTAAAGCCTAGGGTGCCTAATGAGTGAGCTAACTCACATTA
ATTGCGTTGCGCTCACTGCCCGCTTTCCAGTCGGGAAACCTGTCGTGCCAGCTGCATTA
ATGAATCGGCCAACGCGCGGGGAGAGGCGGTTTGCGTATTGGGCGCTCTTCCGCTTCCT
CGCTCACTGACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAACATGTGAGC
AAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAAC
CCGACAGGACTATAAAGATACCAGGCGTTTCCCCCTGGAAGCTCCCTCGTGCGCTCTCC
TGTTCCGACCCTGCCGCTTACCGGATACCTGTCCGCCTTTCTCCCTTCGGGAAGCGTGG
CGCTTTCTCATAGCTCACGCTGTAGGTATCTCAGTTCGGTGTAGGTCGTTCGCTCCAAG
CTGGGCTGTGTGCACGAACCCCCCGTTCAGCCCGACCGCTGCGCCTTATCCGGTAACTA
TCGTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGGCCT
AACTACGGCTACACTAGAAGAACAGTATTTGGTATCTGCGCTCTGCTGAAGCCAGTTAC
CTTCGGAAAAAGAGTTGGTAGCTCTTGATCCGGCAAACAAACCACCGCTGGTAGCGGTG
GTTTTTTTGTTTGCAAGCAGCAGATTACGCGCAGAAAAAAAGGATCTCAAGAAGATCCT
TTGATCTTTTCTACGGGGTCTGACACTCAGTGGAACGAAAACTCACGTTAAGGGATTTT
GGTCATGAGATTATCAAAAAGGATCTTCACCTAGATCCTTTTAAATTAAAAATGAAGTT
TTAAATCAATCTAAAGTATATATGAGTAAACTTGGTCTGACAGTTACCAATGCTTAATC
AGTGAGGCACCTATCTCAGCGATCTGTCTATTTCGTTCATCCATAGTTGCCTGACTCCC
CGTCGTGTAGATAACTACGATACGGGAGGGCTTACCATCTGGCCCCAGTGCTGCAATGA
TACCGCGAGACCCACGCTCACCGGCTCCAGATTTATCAGCAATAAACCAGCCAGCCGGA
AGGGCCGAGCGCAGAAGTGGTCCTGCAACTTTATCCGCCTCCATCCAGTCTATTAATTG
TTGCCGGGAAGCTAGAGTAAGTAGTTCGCCAGTTAATAGTTTGCGCAACGTTGTTGCCA
TTGCTACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGTTAGCTC
CTTCGGTCCTCCGATCGTTGTCAGAAGTAAGTTGGCCGCAGTGTTATCACTCATGGTTA
TGGCAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCTCTTG
CCCGGCGTCAATACGGGATAATACCGCGCCACATAGCAGAACTTTAAAAGTGCTCATCA
TTGGAAAACGTTCTTCGGGGCGAAAACTCTCAAGGATCTTACCGCTGTTGAGATCCAGT
TCGATGTAACCCACTCGTGCACCCAACTGATCTTCAGCATCTTTTACTTTCACCAGCGT
TTCTGGGTGAGCAAAAACAGGAAGGCAAAATGCCGCAAAAAAGGGAATAAGGGCGACAC
GGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGT
TCCGCGCACATTTCCCCGAAAAGTGCCACCTGACGTCGACGGATCGGGAGATCGATCTC
CCGATCCCCTAGGGTCGACTCTCAGTACAATCTGCTCTGATGCCGCATAGTTAAGCCAG
TATCTGCTCCCTGCTTGTGTGTTGGAGGTCGCTGAGTAGTGCGCGAGCAAAATTTAAGC
TACAACAAGGCAAGGCTTGACCGACAATTGCATGAAGAATCTGCTTAGGGTTAGGCGTT
TTGCGCTGCTTCGCGATGTACGGGCCAGATATACGCGTTGACATTGATTATTGACTAGT
TATTAATAGTAATCAATTACGGGGTCATTAGTTCATAGCCCATATATGGAGTTCCGCGT
TACATAACTTACGGTAAATGGCCCGCCTGGCTGACCGCCCAACGACCCCCGCCCATTGA
CGTCAATAATGACGTATGTTCCCATAGTAACGCCAATAGGGACTTTCCATTGACGTCAA
TGGGTGGAGTATTTACGGTAAACTGCCCACTTGGCAGTACATCAAGTGTATC

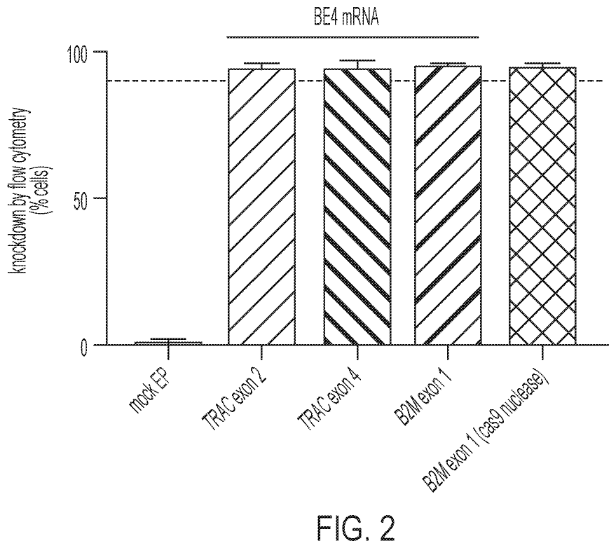

To ascertain the effectiveness of BE4 in knocking down or out protein expression 25 in immune cells, a first population of immune cells was co-transfected with mRNA encoding BE4 and an sgRNA that targeted the C base complementary to the G base of the donor or acceptor splice site of TRAC exon 1, TRAC exon 3, or B2M exon 1, depending on the specific target site. mRNA was produced by in vitro transcription, (TriLin Biotechnologies). Briefly, 4 microgm of BE4 mRNA and 2 microgm of synthetic gRNA were electroporated into 1M CD3+ T cells (Nucleofector™ Platform, Lonza Bioscience). The cells were then cultured for 3 days to allow sufficient time for base-editing. For comparison, a second population of immune cells was co-transfected with mRNA encoding a Cas9 nuclease and sgRNA that target the G base of the donor splice site of B2M exon 1. No discernible difference between BE4 editing and the Cas9 editing was observed, and the knockdown for each edited gene was greater than 90%, whereas unelectroporated control cells had no significant knockdown (FIG. 2).

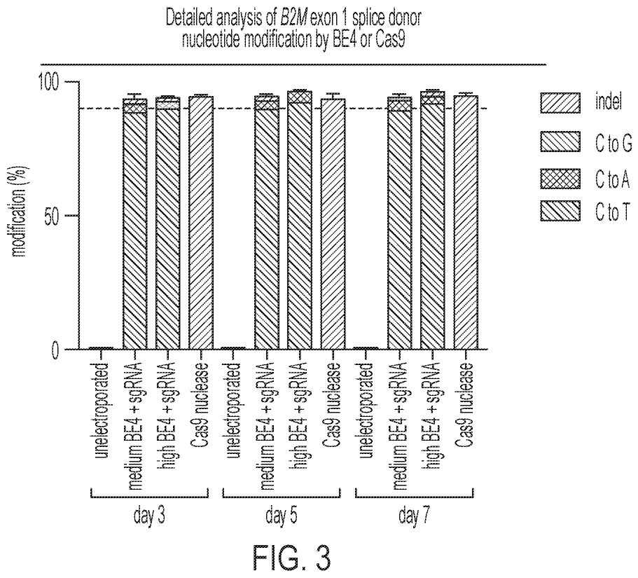

It was hypothesized that the genetic modifications responsible for the observed knockdown of the targeted genes would differ if the cells were transfected with mRNA encoding BE4, which catalyzes single strand nicks, or with the the Cas9 nuclease that catalyzes double-strand breaks. To test this hypothesis, immune cells were co-transfected with either 2 microgm BE4/1 microgm sgRNA (medium) or 4 microgm BE4/2 microgm sgRNA (high) RNA encoding the BE4 base editor and sgRNA that target the G base of the donor splice site of the B2M exon 1. After incubation for 3, 5, and 7 days, DNA was collected and sequenced. Referring to FIG. 3, the majority of base edits revealed disruption of only the splice site and in the manner expected (i.e., C to T transition in the antisense strand was incorporated, resulting in a G to A transition in the sense strand). These results contrasted with those obtained from cells transfected with a Cas9 nuclease, which show that most edits in the Cas9 transfected cells were indels (FIG. 3).

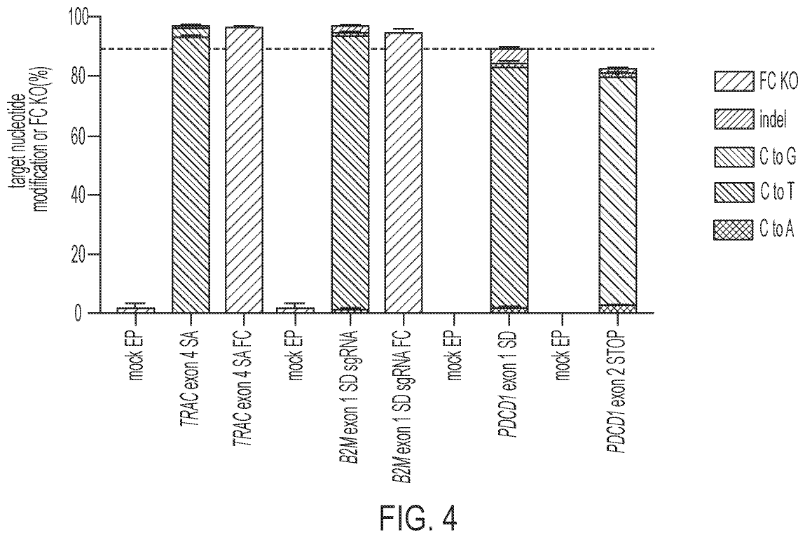

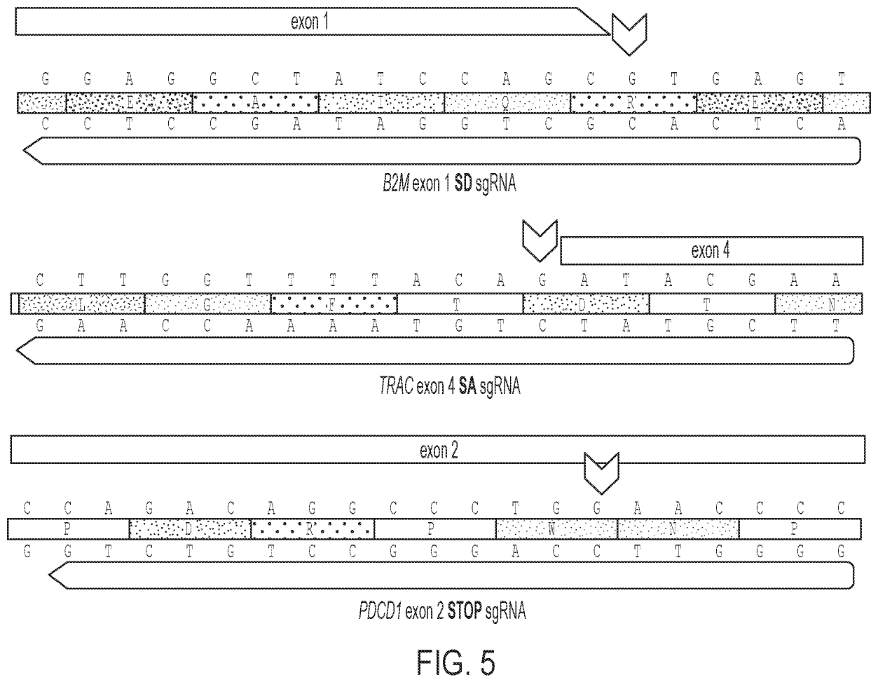

Disruption of splice site and the introduction of stop codons can be effective in knocking down expression of a target gene. BE4-mediated editing of the splice acceptor in TRAC exon 3 and the splice donor in B2M exon 1 and PDCD1 exon 1 resulted in reduced expression of the full-length proteins (FIGS. 4 and 5). The BE4-mediated changes observed in the splice site were C to T transitions, although indels and C to G transversions were also observed. Insertion of an ochre stop codon into exon 2 of the PDCD1 gene, in which consecutive cytidine residues in the exon were targeted and edited to thymidine residues, also resulted in significant knock down of gene expression, albeit a lesser reduction than that seen for the TRAC and B2M genes (FIG. 4). These results further suggest that BE4-mediated single or consecutive cytidine base editing of genes expressed in immune cells results in efficient reduction of gene expression.

### Example 2: In Silico Analysis of Spice Site Disruption and Stop Codon Insertion

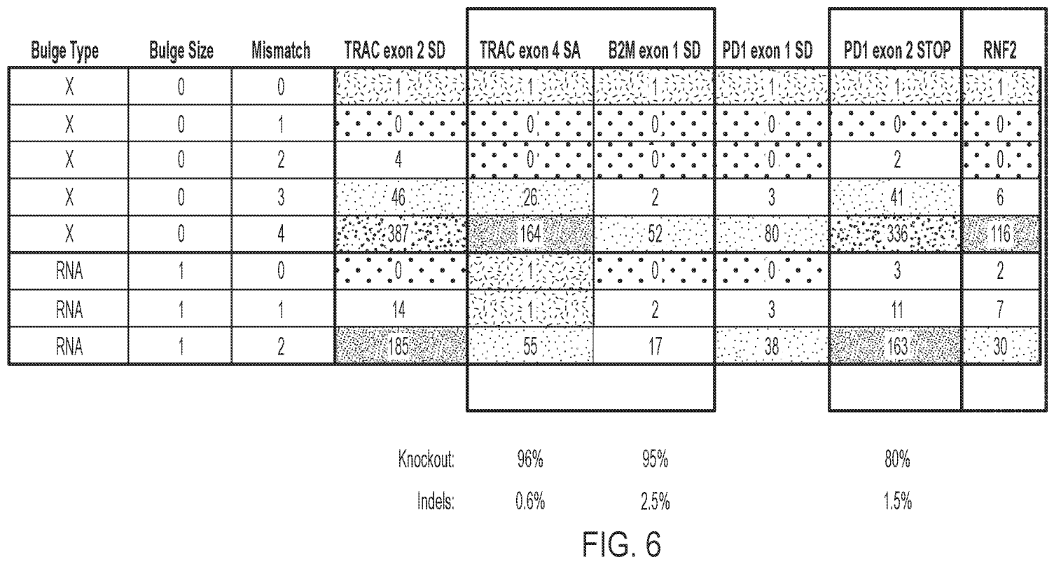

To determine if designed gRNA would bind to off-site targets, the nucleic acid sequences of the gRNAs were analyzed using CAS-OFFinder. Referring to FIG. 6, an “X” bulge type indicates that the gRNA aligns with the genomic DNA and any discrepancy is a mismatch. As the number of mismatches increases from one to four, the potential off-site binding increases. For example, results for the TRAC exon 3 splice acceptor show that when there are three mismatches, there are 26 offsite binding possibilities, while there are 164 with four mismatches.

If the gRNA has a bulge, wherein the gRNA has twenty base pairs, but aligns with nineteen base pairs of genomic DNA, a bulge results. Again referring to FIG. 6, when the TRAC exon 3 splice acceptor gRNA has a bulge of one base pair, the number of offsite binding possibilities increases with increasing mismatches; however, the number of possibilities is significantly lower than when there is no bulge (i.e., when the bulge size is zero).

### Example 3: Multiplex Base Editing in Immune Cells

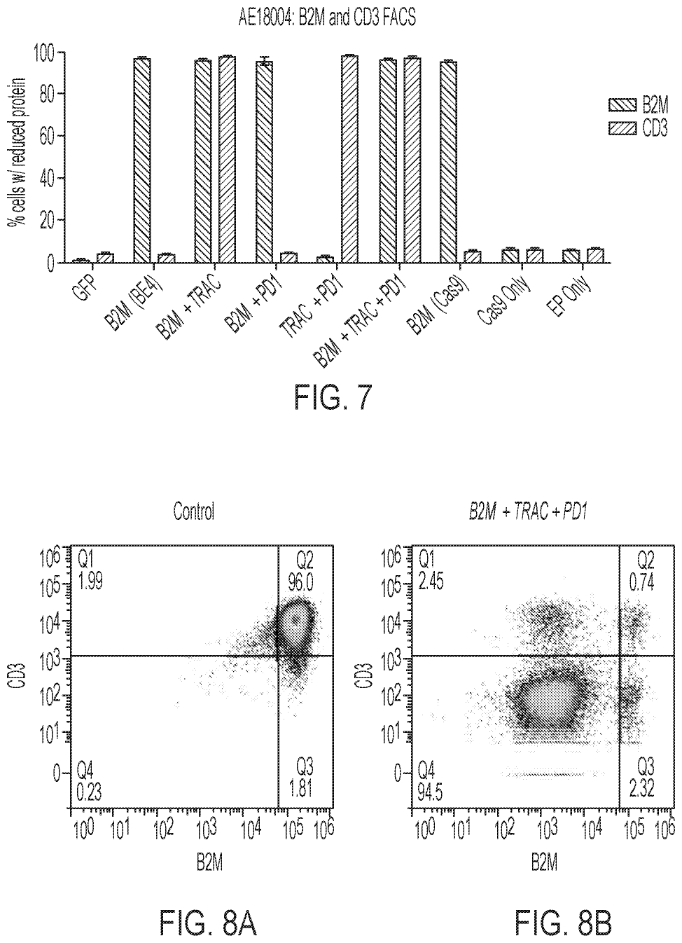

To determine if BE4 could mediate base editing of multiple genes to generate a multi-knockdown cell, immune cells were co-transfected with mRNA encoding a BE4 base editor along with sgRNA that target specific sites in B2M, TRAC, PD1, or in combinations thereof. Referring to FIG. 7, the BE4 system elicited effective knockdown, as measured by flow cytometry, to identify the percentage of cells with decreased protein production in single, double, and triple gene edits. The cells were gated on B2M and CD3 expression, with CD3 expression serving as a proxy for TRAC expression. Because PD1 staining is inefficient, direct measurement of cells expressing this protein was not performed. No differences were observed between cell populations with single, double, and triple gene edits, and immune cells modified to knock-down expression of B2M, TRAC, and PD1 (a triple gene edit) are detectably distinct from non-modified control immune cell (FIG. 8).

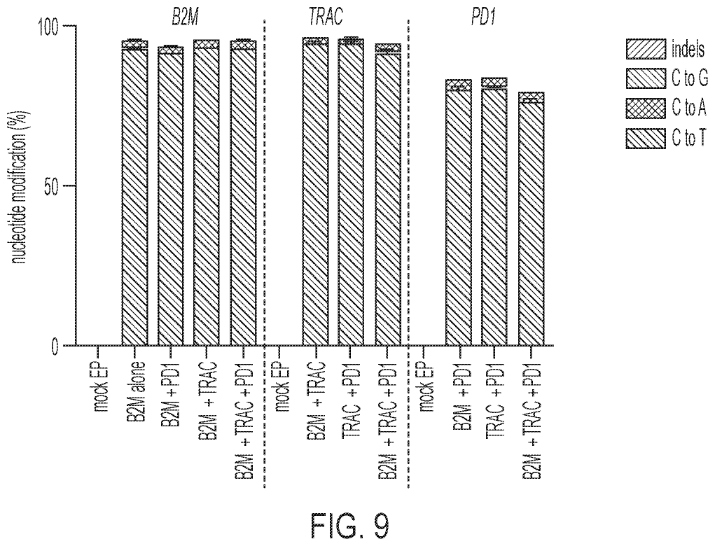

The modifications to the genes responsible for the decreased protein expression are summarized in FIG. 9. Specifically, and similarly to the mechanism resulting in decreased expression in single gene modification described in Example 1, C to T transitions constitute the vast number of edits observed in the modified B2M single modified gene cell population and in the B2M+PD1, B2M+TRAC, and B2M+TRAC+PD1 multiple modified genes cell populations. Indels and transversions constitute an insignificant minority of observed genetic changes in the edited genes.

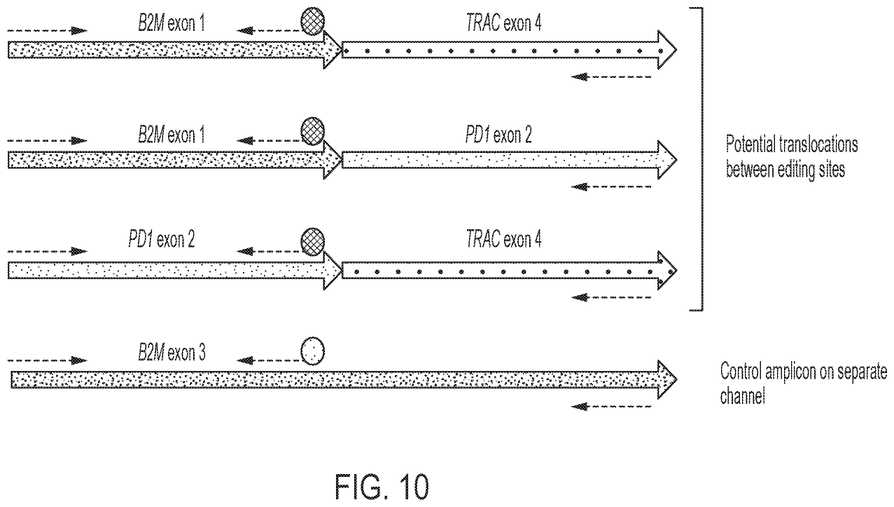

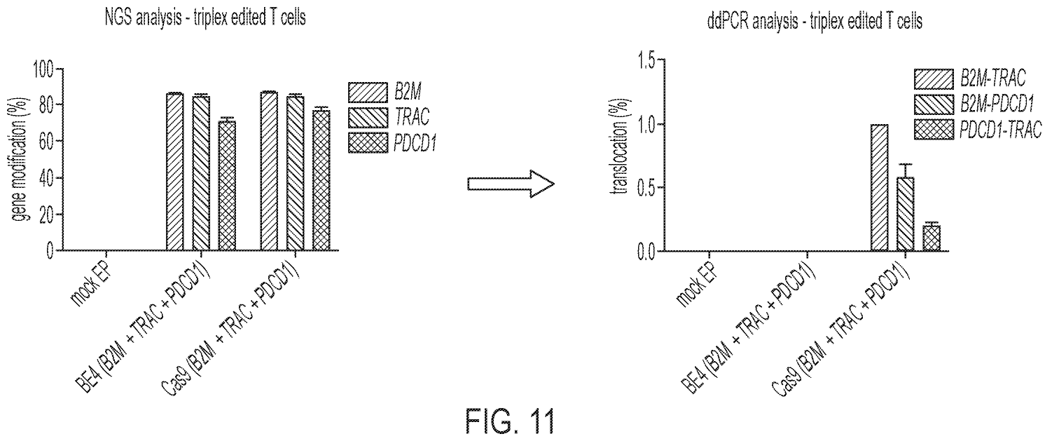

Thus, concurrent modification of three genetic loci by base editing produced highly efficient gene knockouts with no detectable translocation events as assessed by Uni-Directional Targeted Sequencing (UDiTaS; Giannoukos et al., BMC Genomics. 2018 Mar. 21; 19(1):212. doi: 10.1186/s12864-018-4561-9). Additionally, translocations were not detected in BE4-edited genes. A droplet digital polymerase chain reaction (ddPCR) strategy (FIG. 10) was employed to detect translocations between the B2M, TRAC, and PD1 BE4-edited genes. DNA extracted from cells modified with BE4 or Cas9 to generate B2M+TRAC+PD1 edits was analyzed with next generation sequencing (NGS) using a QX200 droplet digital instrument (Bio-Rad) to determine the exact sequence of the BE4 and Cas9 edits. As shown on the left panel of FIG. 11, the B2M, TRAC, and PD1 genes were modified in most cells. ddPCR analysis showed that translocations were not present in the BE4-edited cells, but were observed in approximately 1.7% of the Cas9-edited cells (FIG. 11, right panel). Table 12 further illustrates that translocations were not observed in the BE4-edited cells.

TABLE 12

| | | Control amplicon | Experimental |
|---|---|---|---|
| Base Editor | Translocation | droplets | amplicon droplets |
| Cas9 nuclease | B2M-TRAC | 61,206 | 585 |
| | B2M-PDCD1 | 55,970 | 291 |
| | PDCD1-TRAC | 59,600 | 112 |
| BE4 | B2M-TRAC | 90,717 | 0 |
| | B2M-PDCD1 | 89,028 | 0 |
| | PDCD1-TRAC | 83,501 | 0 |

### Example 4: BE4-Mediated Editing of Cbl Proto-Oncogene B (CBLB)

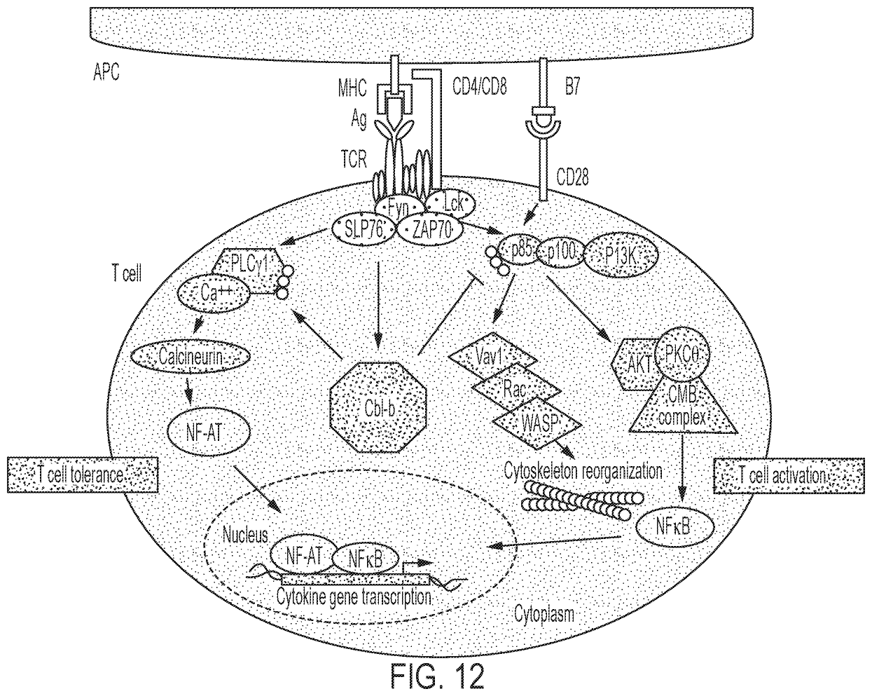

Cbl-b is a T cell receptor (TCR) signaling protein that negatively regulates TCR complex signaling (FIG. 12). Because T cells have a lower activation threshold when Cbl-b signaling is inhibited, knocking out or down this gene could significantly improve the effectiveness of a T cell or a T cell expressing a CAR. To determine if the Cbl-b gene was susceptible cytidine deamination mediated modification, cells were co-transfected with mRNA encoding a BE4 and sgRNA that target the splice site acceptor of exon 8 and 16, the splice site donor of exons 8, 10, 11, and 12, or that would promote the insertion of a STOP codon in exons 1, 4, and 8. Resulting cells were analyzed with flow cytometry.

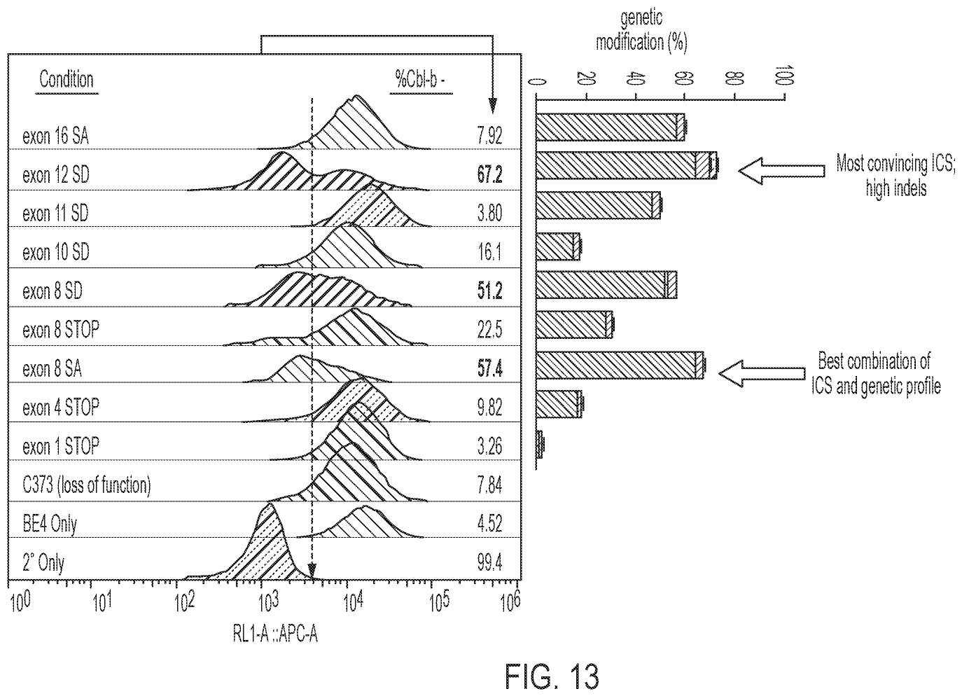

Referring to FIG. 13, disruption of the splice site donor of exon 12 and the splice site acceptor of exon 8 resulted in the greatest reduction of Cbl-b expression (67.2% and 57.4%, respectively). And of the cells transfected with the exon 8 splice site acceptor and the exon 12 splice site donor sgRNAs, slightly more than 60% of the cells were edited successfully (FIG. 13, bar graph).

### Example 5: Cas12b Nuclease Characterization in Immune Cells

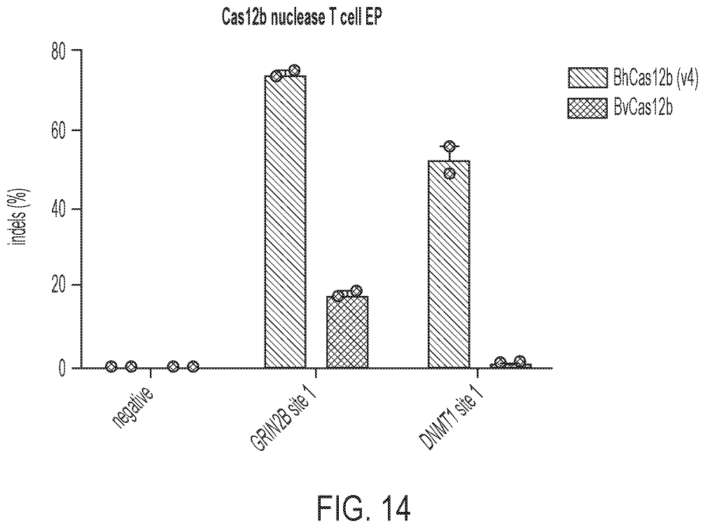

Cas12b/c2c1 site specifically targets and cleaves both strands of a double stranded nucleic acid molecule. Two different Cas12b/c2c1 proteins, BhCas12b and BvCas12b, were characterized by determining the propensity the enzymes for mediating indels in the target nucleic acid molecule. mRNA encoding the Cas12b/c2c1 proteins was electroporated into T cells along with guide RNAs specific for a locus in the GRIN2B gene and for a locus in the DNMT1 gene. The cells were cultured for 3-5 days, followed by isolation of cellular DNA. Indel rates were determined by Next Generation Sequencing. Referring to FIG. 14, DNA isolated from cells treated with the BhCas12b protein had a much higher percentage (approximately 75%) of indels in the GRIN2B gene than did the DNA isolated from cells treated with the BvCas12b protein (approximately 20%). Indels in the DNMT1 gene were also observed at a higher rate in the DNA isolated from cells treated with BhCas12b (approximately 20%) than observed in the DNA isolated from cells treated with BvCas12b (approximately 0%).

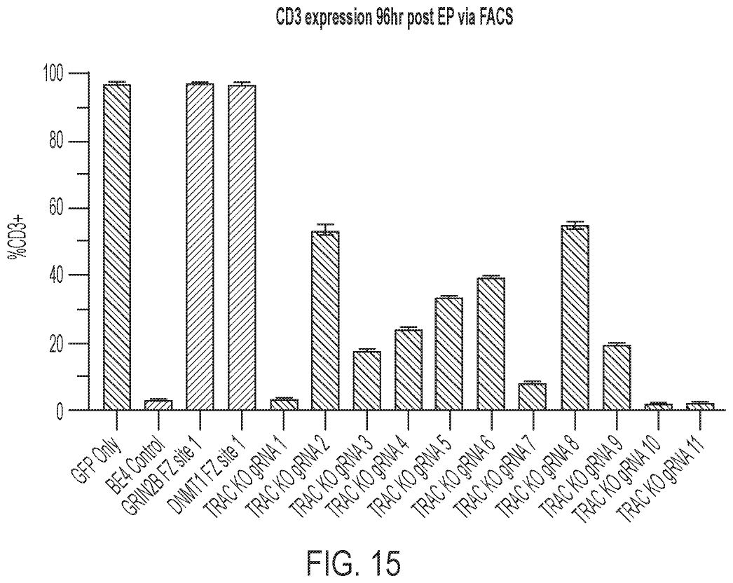

The BhCas12b (V4) protein was used to disrupt the TRAC gene. T cells were transduced via electroporation with the mRNA encoding the BhCas12b (V4) protein along with guide RNAs specific for loci in the GRIN2B, DNMT1, and TRAC genes. 96 hours post-electroporation, cells were assessed using fluorescence assisted cell sorting (FACS) analysis, with cells being gated for CD3 (a proxy for TRAC). Referring to FIG. 15, approximately 95% of T cells transduced with a plasmid encoding GFP or with BhCas12b (V4) and guide RNAs specific for GRIN2B or DNMT1 were CD3+. Those cells transduced to express BhCas12b (V4) and guide RNAs specific for loci in the TRAC gene were less likely to be CD3+ (approximately 2% to approximately 50%, depending on the guide RNA used). Three of the eleven TRAC guide RNAs tested led to approximately 100% BhCas12b (V4)-mediated indels.

### Example 6: CAR-P2A-mCherry Lentivirus Expression Characterization

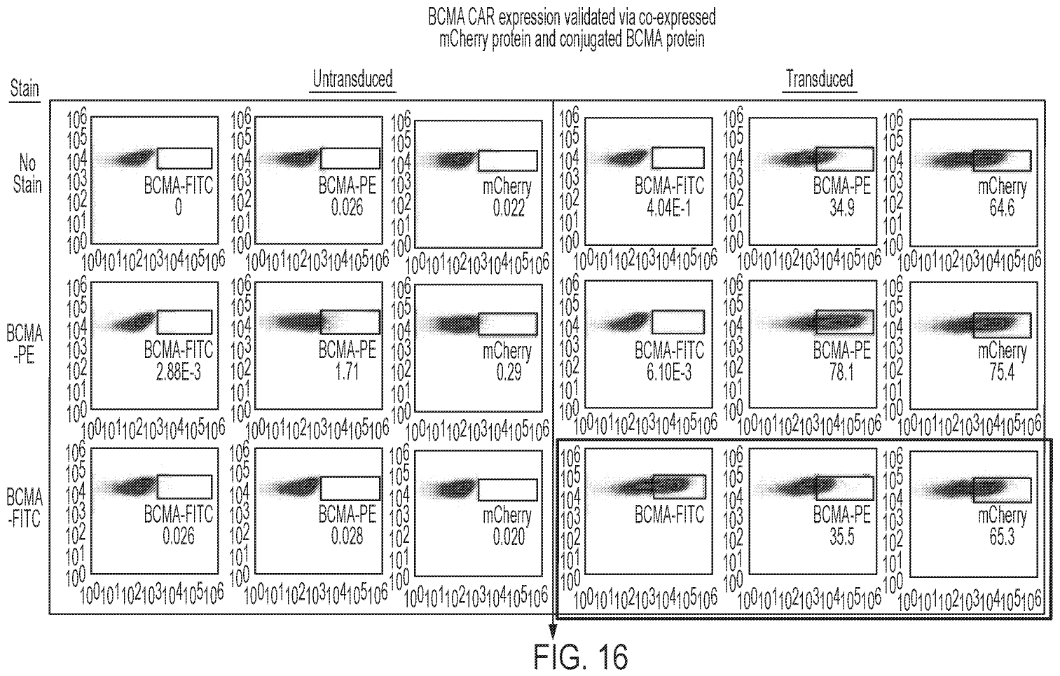

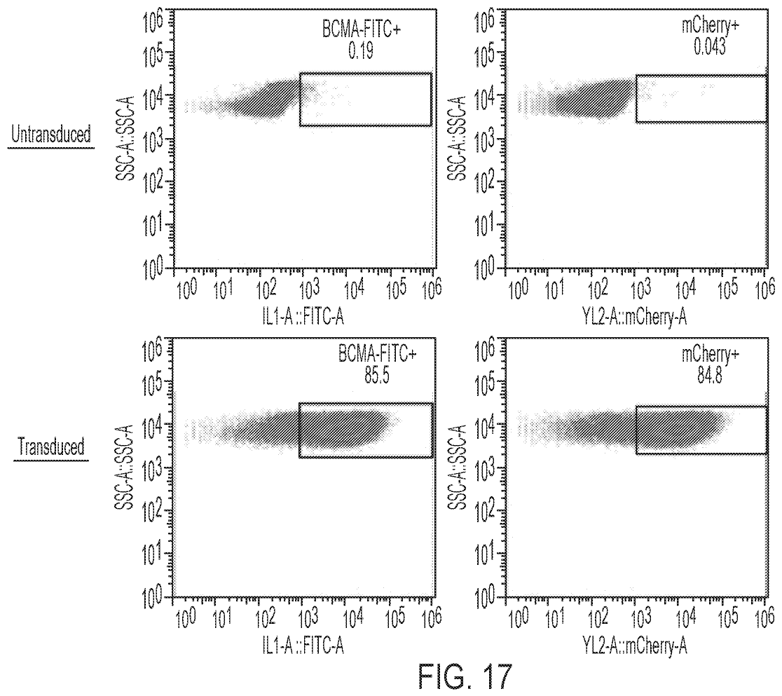

Cells were transduced to express a chimeric antigen receptor (CAR) using the CAR-P2A-mCherry lentivirus and analyzed for CAR expression using fluorescence assisted cell sorting (FACS). Cells were unstained, incubated with a BCMA protein conjugated to R-phycoerythrin (PE) or fluorescein isothiocyanate (FITC). Because BCMA is the CAR's target antigen, cells expressing the CAR will bind dye-labeled BCMA. Referring to FIG. 16, for cells that were not stained, FACS analysis only detected the presence of mCherry in the transduced sample, with some spillover into the PE channel. The BCMA-PE channel shows a highly positive signal beyond what was seen in the spillover, and these results were confirmed in cells incubated with BCMA-FITC. The dye-labeled BCMA protein detection results suggest almost identical expression of the CAR as that seen for mCherry. Referring to FIG. 17, 85% CAR expression was detected via FACS analysis in cells transduced with a poly(1,8-octanediol citrate) (POC) lentiviral vector.

### Example 7: BE4 Produces Efficient, Durable Gene Knockout with High Product Purity

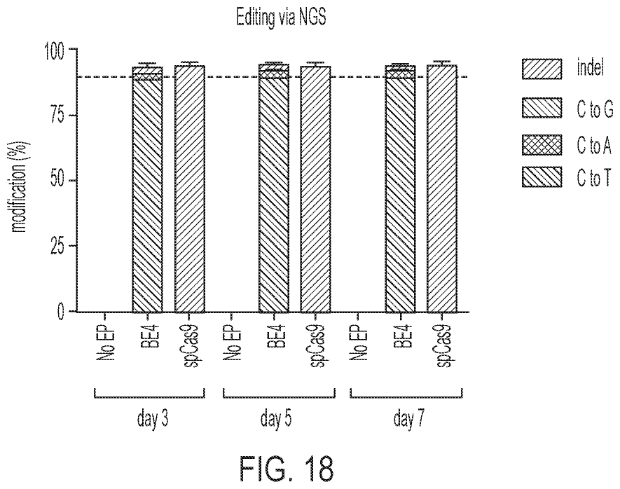

BE4 mediates base editing of multiple genes to generate a multi-knockdown cell. Immune cells were co-transfected with mRNA encoding a BE4 base editor along with sgRNA that target specific sites in B2M, TRAC, PD1, or in combinations thereof. As shown by sequencing data, base editing was efficient at modifying cells and durable up to at least 7 days (FIG. 18). High product purity was observed, as C to T transitions constituted the vast number of edits observed. Indels and C-to-G and C-to-A transversions constituted an insignificant minority of observed genetic changes in the edited genes. Base editing was also as efficient as spCas9 nuclease at generating desired modifications.

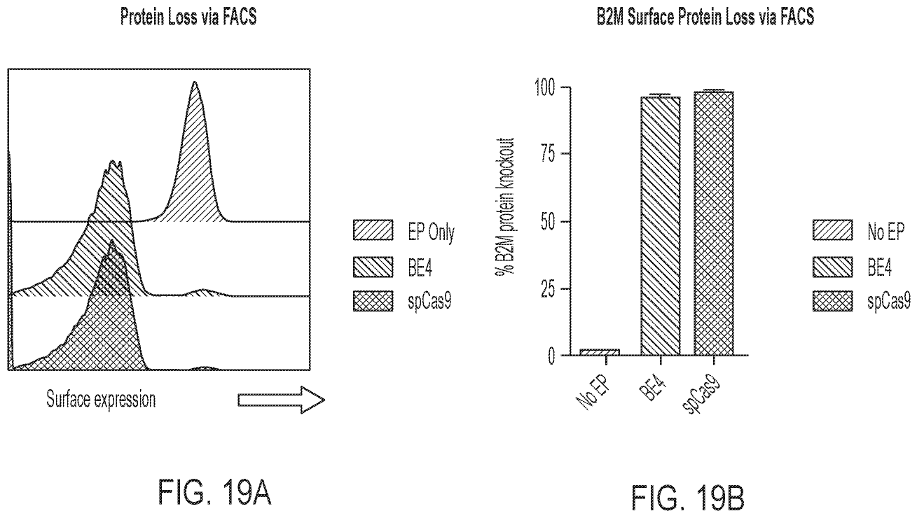

The BE4 system elicited effective knockdown as measured by flow cytometry, which identifies the percentage of cells with decreased surface expression (FIG. 19A). Cells gated on B2M expression displayed loss of B2M protein on the cell surface. As measured by flow cytometry, base editing was also as efficient as spCas9 nuclease at generating B2M protein knockout.

### Example 8: Orthogonal Translocation Detection Assay Cannot Detect BE4-Induced Rearrangements in Triple-Edited T Cells

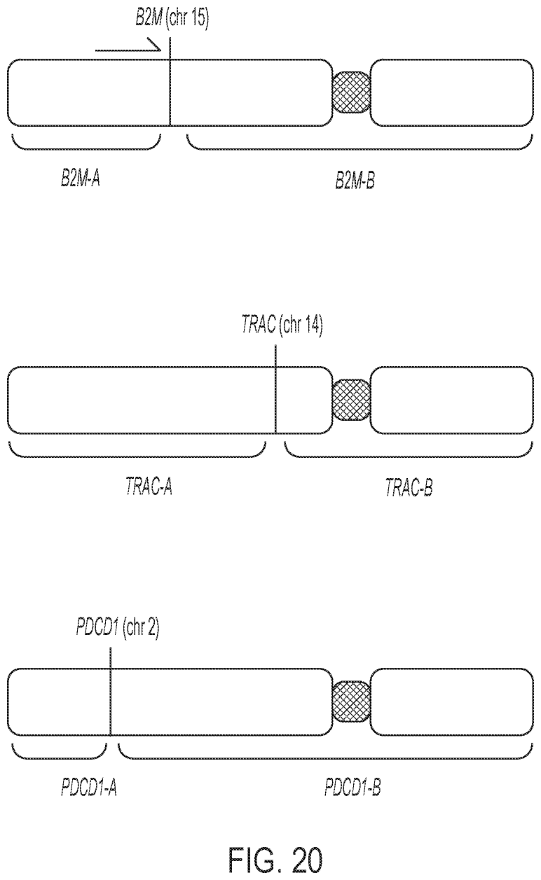

Immune cells were co-transfected with mRNA encoding a BE4 base editor along with sgRNAs that targeted specific sites in B2M, TRAC, and PD1. The triple-edited T cells were evaluated using a translocation detection assay that was capable of detecting specific translocations that were undesirable between B2M, TRAC, and PD1 target genes (FIG. 20). Notably, none of these specific translocations were detected in any of the BE4-edited genes (Table 13). In contrast, Cas9-treated cells displayed low, but detectable levels of the translocations. Thus, multiplex editing of T cells using the BE4 base editor did not generate translocations in contrast to multiplex editing using Cas9 nuclease.

TABLE 13

| | Mock | BE4-treated | Cas9-treated |
|---|---|---|---|
| Type | (%) | (%) | (%) |
| On-target modification | 0 | 89.9/97.9/89.1 | 53.0/77.2/55.2 |
| (B2M/TRAC/PDCD1) | | | |
| B2M-A/TRAC-A | 0 | 0 | 0.925 |
| B2M-A/TRAC-B | 0 | 0 | 0.353 |
| B2M-A/PDCD1-A | 0 | 0 | 1.647 |
| B2M-A/PDCD1-B | 0 | 0 | 0.508 |
| B2M-B/TRAC-A* | 0 | 0 | 0.505 |

LLoD_{BE4} = 0.1%
*B2M-B only measurable in this experiment if translocation includes a local rearrangement at the B2M locus

### Example 9: Multiplexed Base Editing does not Significantly Impair Cell Expansion

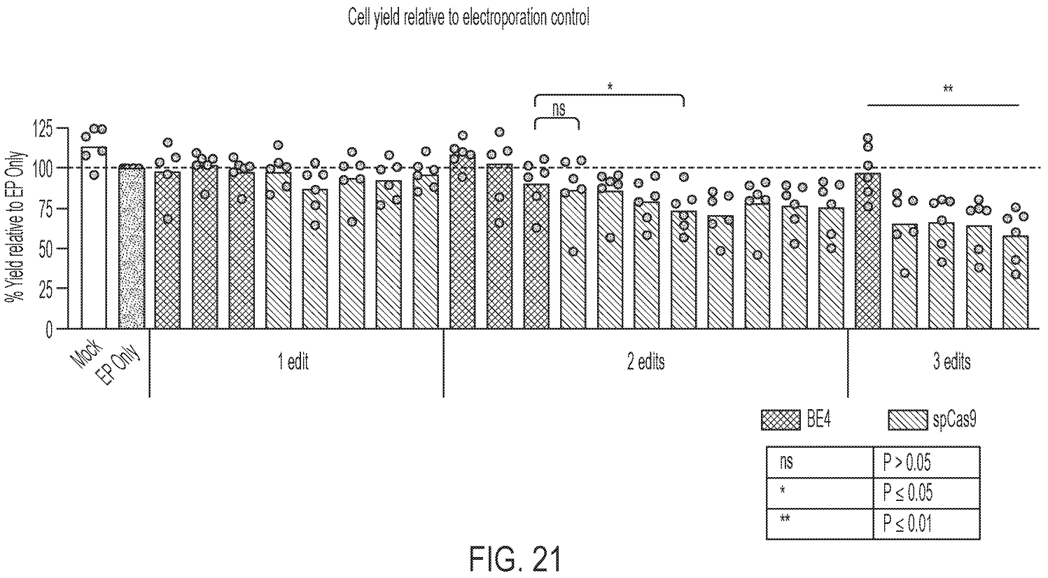

An extensive guide screen was performed across B2M, TRAC, and PD1 targets with both BE4 and spCas9 sgRNAs. Guides were selected for high editing efficiency and expansion based on single-plex test. Final cell yields compared between 1, 2 and 3 edits using BE4 and spCas9 and were normalized to electroporation only control. BE4 edited cells with the desired edits displayed high yields when up to 3 edits were made (FIG. 21). In contrast, spCas9 edited cells showed reduced yields when increasing numbers of multiplex edits were made. Thus, multiplexed base edited cells maintained high cell expansion even when up to 3 edits were being made. Thus, BE4 generated multiplex-edited T cells with no detectable genomic rearrangements while also maintaining high cell expansion compared to spCas9 treated samples.

### Example 10: BE4 Generated Triple-Edited T Cells with Similar On-Target Editing Efficiency and Cellular Phenotype as spCas9

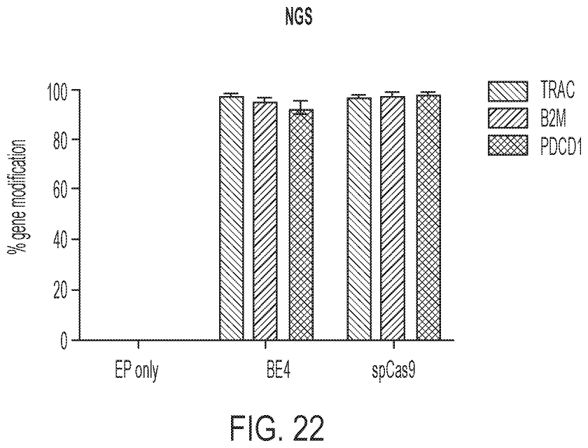

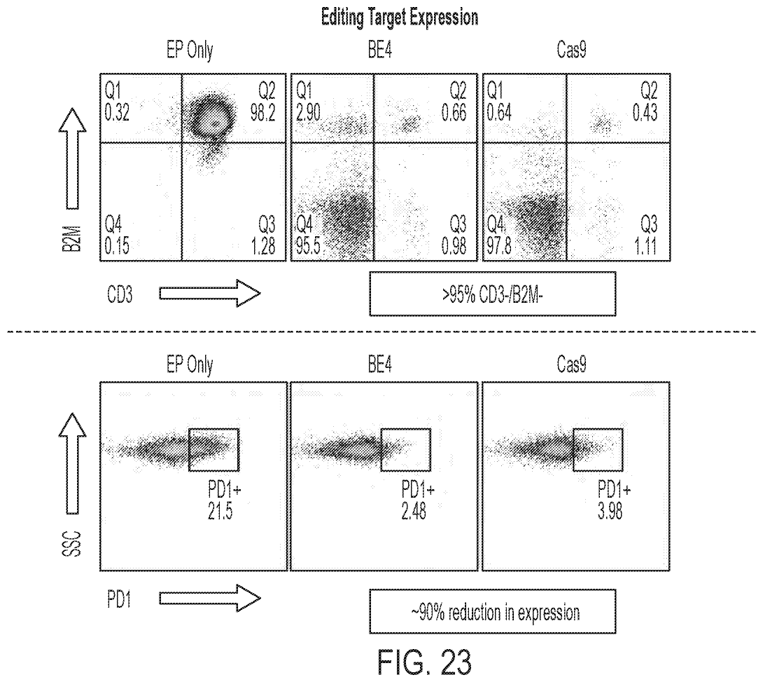

T cells were co-transfected with mRNA encoding a BE4 base editor along with sgRNAs that target specific sites in B2M, TRAC, and PD1. As shown by sequencing data, base editing was efficient at modifying cells at all three sites (FIG. 22). Modification of the genes by base editing was similar to that using spCas9 nuclease. Flow cytometry also showed decreased surface expression of B2M and CD3 (FIG. 23, upper panel). Compared to electroporation only control cells, BE4 and Cas9 multiplex edited cells displayed significant reductions of B2M and CD3 protein on the cell surface (>95% CD3⁻/B2M⁻). Although PD1 staining is less efficient, significant reductions (˜90%) in PD1 were observed in BE4 and Cas9 multiplex edited cells compared to electroporation only control cells (FIG. 23, lower panel).

### Example 11: BE4 Editing does not Alter CAR Expression or Antigen-Dependent Cell Killing

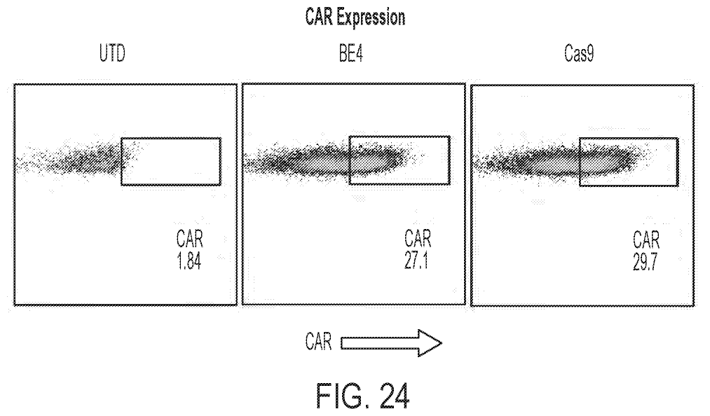

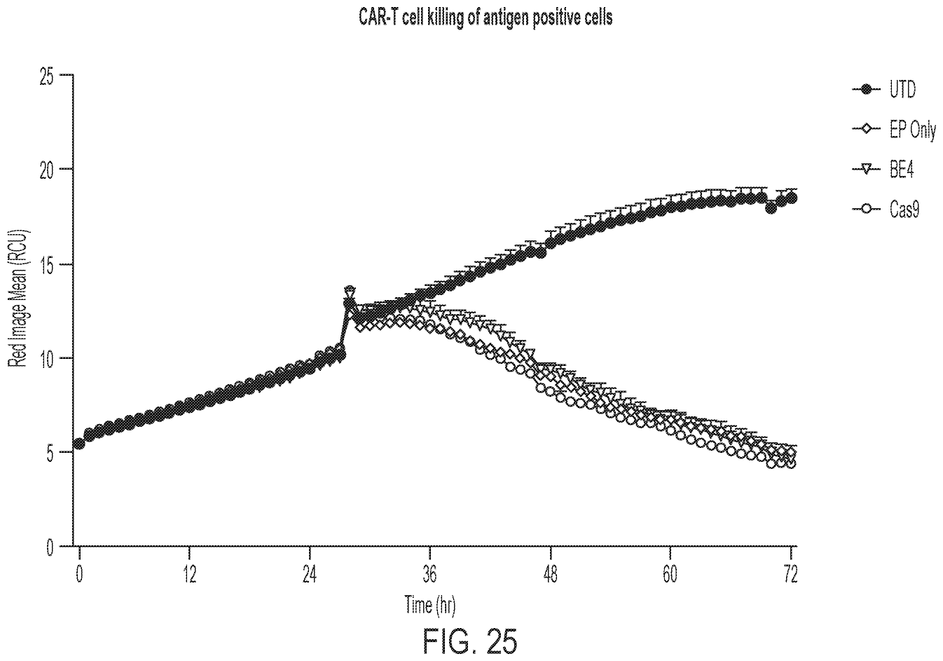

T cells were co-transfected with mRNA encoding a BE4 base editor along with sgRNAs that target specific sites in B2M, TRAC, and PD1. A chimeric antigen receptor (CAR) targeting BCMA was introduced by integration of a lentiviral vector encoding the anti-BCMA CAR. CAR expression was observed by flow cytometry in BE4 and Cas9 edited cells (FIG. 24), compared to untreated cells that did not receive the lentiviral vector. The CAR-T cells were evaluated for cell killing by nuclear staining of the cells expressing BCMA and detecting loss of nuclear staining, indicating cell death. Antigen dependent cell killing was observed in cells transduced with the vector and expressing the CAR, including BE4 and Cas9 edited T cells (FIG. 25). In contrast, untreated cells that were not transduced with the vector did not display cell killing activity. Thus, BE4-generated CAR-T cells demonstrated comparable gene disruption, cell phenotype, and antigen-dependent cell killing compared to their nuclease-only counterparts.

### Example 12: Cas12b and BE4 can be Paired for Highly Efficient Multiplex Editing in T Cells

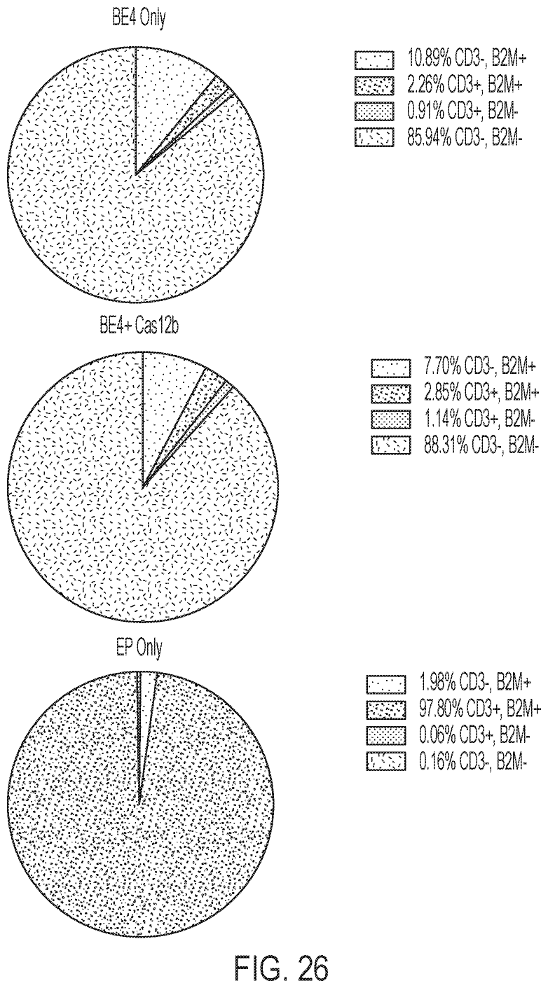

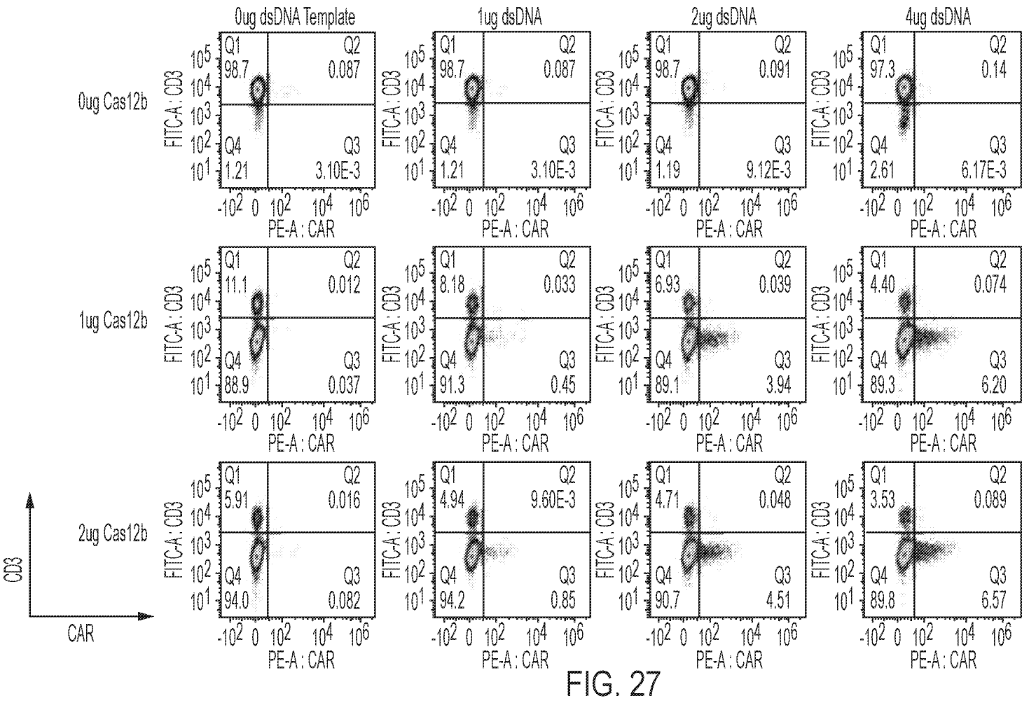

CD3⁻, B2M⁻ T cells were generated using BE4 only or using BE4 and Cas12b. For T cells generated using BE4 only, T cells were co-transfected with mRNA encoding a BE4 base editor along with sgRNAs that target specific sites in B2M and TRAC. For T cells generated using BE4 and Cas12b, T cells were co-transfected with mRNA encoding a BE4 base editor, and an sgRNA that targets a specific site in B2M, mRNA encoding BhCas12b (V4), and a Cas12b sgRNA that targets exon 3 of the TRAC gene, which was used to disrupt the TRAC gene. The resulting T cells were assessed using fluorescence assisted cell sorting (FACS) analysis to detect B2M and CD3 cell surface expression. Knockouts using BE4 only displayed a similar profile to those using BE4 and Cas12b. In particular, a high percentage of the T cells were CD3⁻, B2M⁻: 86% (BE4 only) and 88% (BE4+Cas12b), while the other possible phenotypes CD3⁻, B2M⁺; CD3⁺, B2M⁺ T cells; and CD3⁺, B2M⁻ were represented less in the cell population (FIG. 26). In contrast, electroporation only control showed a population having a high percentage (97.8%) of CD3⁺ B2M⁺ cells and a very low percentage of CD3⁻, B2M⁻ cells.

Cas12b was used to generate CD3⁻, CAR⁺ T cells. T cells were co-transfected with mRNA encoding BhCas12b (V4), a Cas12b sgRNA that targets exon 3 of the TRAC gene, and a double-stranded DNA (dsDNA) donor template encoding BCMA02, an anti-BCMA CAR. T cells were assessed using fluorescence assisted cell sorting (FACS) analysis to detect CD3 and BCMA02 cell surface expression. When increasing amounts of Cas12b were introduced into the cell in the presence of the sgRNA, CD3 expression decreased, as seen by a shift in the cell population to the CD3⁻ quadrant (FIG. 27). When increasing amounts of donor template and were introduced in the cells under the same conditions, a shift to CD3⁻, CAR⁺ quadrant was observed in the cell population.

Thus, Cas12b can be paired with BE4 to generate multiplex-edited T cells, minimizing genomic rearrangements caused by multiple double-strand breaks.

### Example 13: High Efficiency Multiplex Knockout of Eight Targets

In this example, PBMCs were isolated from three donors and activated with soluble CD3 and CD28 antibodies. On day 3 after activation, T cells were electroporated with a reaction mixture including 2 microgm of recombinant BE4 and 1 microgm each of sgRNAs using a LONZA 4D electroporation device. (see Table 10 for sgRNA electroporated). Where indicated, half (½) gRNA dose is 0.5 microgm each of sgRNA; and 2× mRNA dose=4 microgm mRNA with 0.5 microgm of each sgRNA. sgRNA were obtained from Synthego or Agilent.

Percent knockdown of gene expression was measured by flow cytometry. To determine the base editing efficiency of CIITA gene, HLADR was used as the surrogate protein for staining. These results indicate that efficient and effective multiplex base editing can be successfully performed on a large number of genes simultaneously in single electroporation events.

TABLE 14

| Target | Target Sequence |
|---|---|
| CD3 | TTCGTATCTGTAAAACCAAG |
| CD7 | CCTACCTGTCACCAGGACCA |
| CD52 | CTCTTACCTGTACCATAACC |
| PD1 | CACCTACCTAAGAACCATCC |
| B2M | ACTCACGCTGGATAGCCTCC |
| CD5 | ACTCACCCAGCATCCCCAGC |
| CIITA | CACTCACCTTAGCCTGAGCA |
| CD2 | CACGCACCTGGACAGCTGAC |

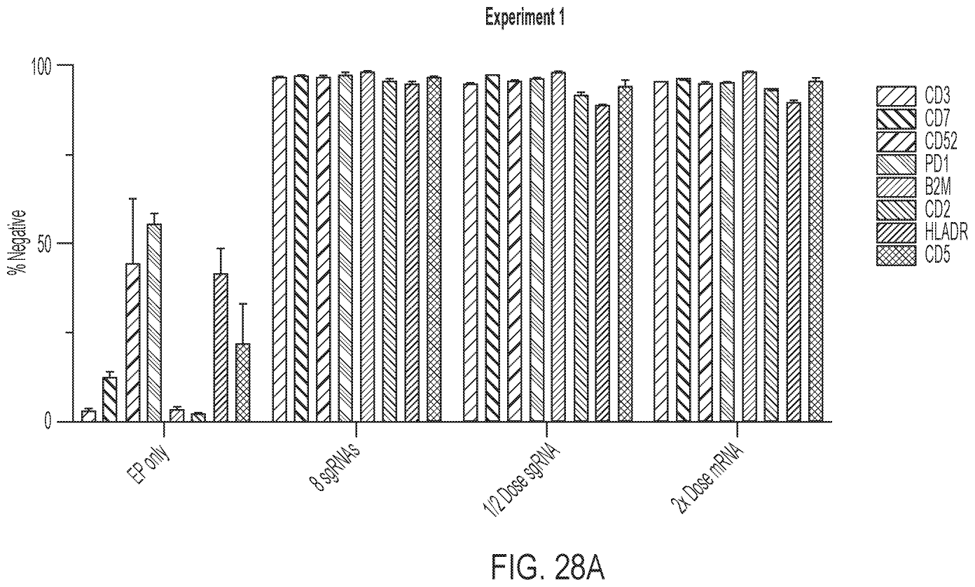

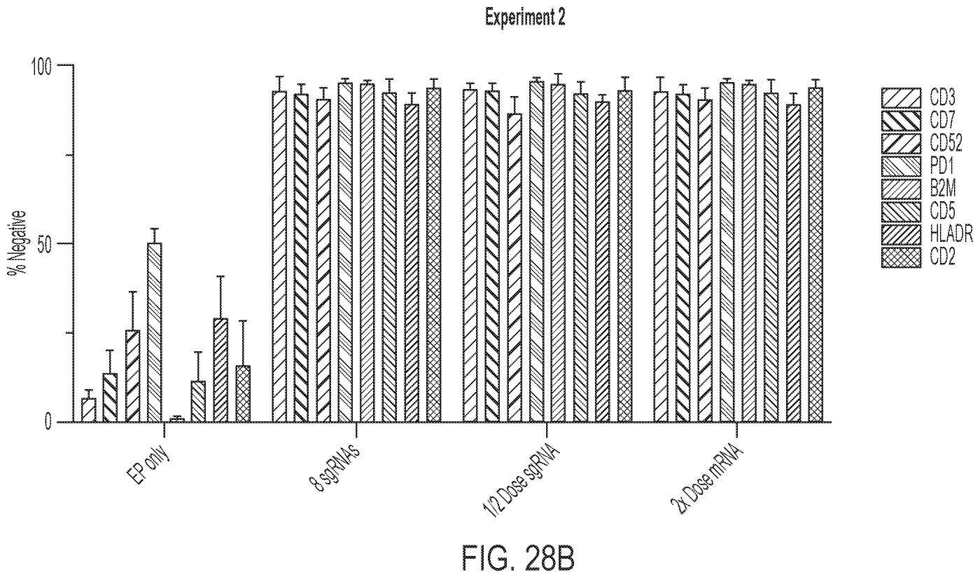

As indicated in FIG. 28A and FIG. 28B, knockdown of each of the targeted genes was achieved.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for producing a modified immune cell with reduced immunogenicity and/or increased anti-neoplasia activity by multiplexed editing, the method comprising: modifying a target nucleobase in at least four genes or regulatory elements thereof in an immune cell, thereby generating the modified immune cell with reduced immunogenicity and/or increased anti-neoplasia activity.

2. (canceled)

3. The method of claim 1, wherein at least one of the four genes is a checkpoint inhibitor gene, an immune response regulation gene, or an immunogenic gene.

4. (canceled)

5. The method of claim 1, wherein expression of at least one of the four genes is reduced by at least 80% as compared to a control cell without the modification.

6-8. (canceled)

9. The method of claim 1, wherein the four genes encode polypeptides that form a TCR complex.

10. The method of claim 1, wherein one of the four genes encodes a polypeptide selected from the group consisting of TRAC, a check point inhibitor, PDCD1, a T cell marker, CD52, CD7, CD3 epsilon, CD3 gamma, CD3 delta, TRBC1, TRBC2, CD4, CD5, CD7, CD30, CD33, CD52, CD70, B2M, and CIITA.

11-28. (canceled)

29. The method of claim 1, wherein the modifying comprises deaminating the single target nucleobase.

30. The method of claim 29, wherein the deaminating is performed by a polypeptide comprising a deaminase.

31. The method of claim 30, wherein the deaminase is associated with a nucleic acid programmable DNA binding protein (napDNAbp) to form a base editor.

32. The method of claim 31, wherein the deaminase is fused to the nucleic acid programmable DNA binding protein (napDNAbp).

33. (canceled)

34. The method of claim 32, wherein the napDNAbp comprises a Cas9 nickase or nuclease dead Cas9.

35. The method of claim 32, wherein the deaminase is a cytidine deaminase that converts a cytosine to a thymine or an adenosine deaminase that converts an adenosine (A) to a guanine (G).

36. (canceled)

37. The method of claim 35, wherein the base editor further comprises a uracil glycosylase inhibitor.

38-42. (canceled)

43. The method of claim 40, wherein the modifying comprises contacting the immune cell with a base editor and a guide nucleic acid sequence comprising a sequence selected from the group consisting of UUCGUAUCUGUAAAACCAAG, CCUACCUGUCACCAGGACCA, CUCUUACCUGUACCAUAACC, CACCUACCUAAGAACCAUCC, ACUCACGCUGGAUAGCCUCC, ACUCACCCAGCAUCCCCAGC, CACUCACCUUAGCCUGAGCA, and CACGCACCUGGACAGCUGAC.

44-55. (canceled)

56. The method of claim 1, wherein the single target nucleobase is in an exon, a splice donor site or a splice acceptor site.

57. The method of claim 1, wherein the target nucleobase is in a splice acceptor or splice donor of a TRAC, PDCD1, CD52, CD7, B2M, CD2, CD5, or CIITA gene.

58-64. (canceled)

65. The method of claim 1, wherein the immune cell is a human cytotoxic T cell, a regulatory T cell, a T helper cell, a dendritic cell, a B cell, or a NK cell.

66-69. (canceled)

70. The method of claim 1, wherein the immune cell is derived from a single human donor.

71. The method of claim 1, further comprising contacting the immune cell with a lentivirus comprising a polynucleotide that encodes an exogenous functional chimeric antigen receptor (CAR) or a functional fragment thereof.

72-74. (canceled)

75. The method of claim 71, wherein the CAR specifically binds a marker associated with neoplasia.

76. The method of claim 75, wherein the neoplasia is a T cell cancer, a B cell cancer, a lymphoma, a leukemia, or a multiple myeloma.

77. The method of claim 76, wherein the CAR specifically binds CD7 or BCMA.

78-85. (canceled)

86. A modified immune cell produced according to the method of claim 1.

87. (canceled)

88. A modified immune cell with reduced immunogenicity or increased anti-neoplasia activity, wherein the modified immune cell comprises a single target nucleobase modification in each one of at least four gene sequences or regulatory elements thereof, wherein the gene sequences are selected from the group consisting of CD3, CD5, CD52, CD7, CD2, TRAC, CD3 epsilon, CD3 gamma, CD3 delta, TRBC1, TRBC2, CD4, CD30, CD33, CD70, B2M, and CIITA or a regulatory element of each thereof, and the immune cell is a human immune cell selected from the group consisting of a cytotoxic T cell, a regulatory T cell, a T helper cell, a dendritic cell, a B cell, and a NK cell.

89-177. (canceled)

178. A composition comprising a base editor comprising a nucleic acid programmable DNA binding protein (napDNAbp), a deaminase, and a uracil glycosylate inhibitor, and a guide nucleic acid sequence, wherein the guide nucleic acid sequence comprises a sequence selected from the group consisting of UUCGUAUCUGUAAAACCAAG, CCUACCUGUCACCAGGACCA, CUCUUACCUGUACCAUAACC, CACCUACCUAAGAACCAUCC, ACUCACGCUGGAUAGCCUCC, ACUCACCCAGCAUCCCCAGC, CACUCACCUUAGCCUGAGCA, and CACGCACCUGGACAGCUGAC.

179. (canceled)

180. The composition of claim 178, wherein the napDNAbp comprises a Cas9 nickase or nuclease dead Cas9 and wherein the deaminase is a cytidine or adenosine deaminase.

181-184. (canceled)

185. A method for producing a modified immune cell with reduced immunogenicity and/or increased anti-neoplasia activity, the method comprising:
a) modifying a single target nucleobase in a first gene sequence or a regulatory element thereof in an immune cell;
b) modifying a second gene sequence or a regulatory element thereof in the immune cell with a Cas12 polypeptide, wherein the Cas12 polypeptide generates a site-specific cleavage in the second gene sequence; wherein each of the first gene and the second gene is an immunogenic gene, a checkpoint inhibitor gene, or an immune response regulation gene; and
c) contacting the modified immune cell with a lentivirus comprising a polynucleotide encoding an exogenous functional chimeric antigen receptor (CAR) or a functional fragment thereof, thereby generating a modified immune cell with reduced immunogenicity and/or increased anti-neoplasia activity.

186. (canceled)

187. The method of claim 185, wherein the polynucleotide encoding the CAR or the functional fragment thereof is inserted into the site specific cleavage generated by the Cas12 polypeptide.

188. (canceled)

189. The method of claim 185, wherein each of the first gene and the second gene is an immunogenic gene, a checkpoint inhibitor gene, or an immune response regulation gene.

190-220. (canceled)

221. A modified immune cell with reduced immunogenicity and/or increased anti-neoplasia activity, the modified immune cell comprising:
a) a single target nucleobase modification in a first gene sequence or a regulatory element thereof in an immune cell; and
b) a modification in a second gene sequence or a regulatory element thereof, wherein the modification is an insertion of an exogenous chimeric antigen receptor (CAR) or a functional fragment thereof or an exogenous T cell receptor or a functional fragment thereof;
wherein each of the first gene and the second gene is a immunogenic gene, a checkpoint inhibitor gene, or immune response regulation gene.

222-263. (canceled)

264. A method for producing a modified immune cell with increased anti-neoplasia activity, the method comprising: modifying a single target nucleobase in a Cbl Proto Oncogene B (CBLB) gene sequence or a regulatory element thereof in an immune cell, wherein the modification reduces an activation threshold of the immune cell compared with an immune cell lacking the modification; thereby generating a modified immune cell with increased anti-neoplasia activity.

265. A composition comprising the modified immune cell of claim 264.

266-267. (canceled)

268. A composition comprising a polynucleotide encoding a base editor polypeptide, wherein the base editor polypeptide comprises a nucleic acid programmable DNA binding protein (napDNAbp) and an adenosine or cytidine deaminase and at least four different guide nucleic acid sequences for base editing.

269-277. (canceled)

278. An immune cell comprising the composition of claim 268, wherein the composition is introduced into the immune cell with electroporation, nucleofection, viral transduction, or a combination thereof.

279-283. (canceled)
